(19) United States
(12) Patent Application Publication
(10) Pub. No.: US 2025/0092031 A1
(43) Pub. Date: Mar. 20, 2025

(54) RIPK3 INHIBITORS AND METHODS OF USE

(71) Applicant: Momentum Therapeutics, Inc., New York, NY (US)

(72) Inventors: Balaji Dashrath SATHE, Greater Noida (IN); Nancy SAJWAN, Bathinda (IN); Gonzalo Andrés URETA DÍAZ, Santiago (CL); Sebastián BERNALES, Piedmont, CA (US); Dayanand PANPATIL, Gautam Buhd nagar (IN); Sebastián BELMAR, Santiago (CL)

(21) Appl. No.: 18/891,613

(22) Filed: Sep. 20, 2024

Related U.S. Application Data
(60) Provisional application No. 63/540,550, filed on Sep. 26, 2023.
(60) Provisional application No. 63/539,537, filed on Sep. 20, 2023.

(51) Int. Cl. C07D 417/12; A61K 31/4709; A61K 31/496; A61K 31/5377; C07D 215/233; C07D 215/42; C07D 401/10; C07D 401/12; C07D 401/14; C07D 405/12; C07D 405/14; C07D 409/14; C07D 413/14; C07D 417/14; C07D 471/08; C07D 471/10; C07D 487/04; C07D 487/08; C07D 487/10; C07D 491/107

(52) U.S. Cl. CPC C07D 417/12; A61K 31/4709; A61K 31/496; A61K 31/5377; C07D 215/233; C07D 215/42; C07D 401/10; C07D 401/12; C07D 401/14; C07D 405/12; C07D 405/14; C07D 409/14; C07D 413/14; C07D 417/14; C07D 471/08; C07D 471/10; C07D 487/04; C07D 487/08; C07D 487/10; C07D 491/107

(57) ABSTRACT

The present disclosure in some aspects relates generally to compounds for inhibiting RIPK3 and/or providing a therapeutic effect over a range of conditions, compositions thereof, and uses thereof.

### CROSS-REFERENCE TO RELATED APPLICATIONS

This application claims priority to and benefit of U.S. Provisional Patent Application No. 63/539,537 filed Sep. 20, 2023, and U.S. Provisional Patent Application No. 63/540,550, filed Sep. 26, 2023, the disclosures of which are hereby incorporated herein by reference in their entireties.

### FIELD

The present disclosure relates generally to compounds and compositions for inhibiting receptor-interacting protein kinase 3 (RIPK3) and methods of making and using the same.

### BACKGROUND OF THE INVENTION

Necroptosis is a type of programmed cell death that has been implicated in various human diseases. This form of cell death is characterized by a regulated, inflammatory response that is distinct from other cell death programs such as apoptosis. Necroptosis and apoptosis also differ in their activation pathways, although there are some common molecular players of both types of programmed cell death.

Different stimuli induce necroptosis activation, including TNF ligand family members, interferons, and activation of toll-like receptors (Grootjans et al., Cell Death Differ, 24:1184-1195, 2017). In the most canonical type of necroptosis activation, stimulation of the TNF type 1 receptor (TNFR1) leads to the recruitment of receptor-interacting protein kinase 1 (RIPK1), and trigger the activation of either caspase-8 or RIPK3, resulting in cell apoptosis or necroptosis, respectively (Schwarzer et al. Curr Opin Cell Biol, 63:186-193, 2020). Following a stimulus such as TNF-α ligation, and in the absence of caspase-8 activity, the interaction of RIPK1 and RIPK3 induces the formation of the necrosome complex and RIPK3 phosphorylation, a key event in necroptosis activation (He S et al., Cell, 137:1100-1111, 2009). RIPK3 activation leads to the recruitment and phosphorylation of the pseudokinase mixed lineage kinase domain-like protein (MLKL) within the necrosome complex. Upon activation, MLKL oligomerizes and translocates to the plasma membrane, and executes necroptosis leading to cell death (Cai Z et al., Nat Cell Biol, 16:55-65, 2013).

A potent and selective inhibitor of RIPK3 will block RIPK3-dependent necroptosis activation in several conditions and human diseases, providing a therapeutic effect over a range of conditions.

### SUMMARY

In one aspect, provided herein is a compound of Formula (Ia):

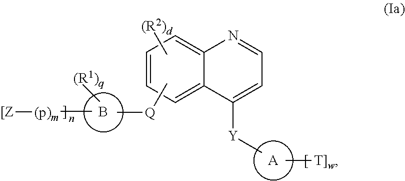

or a pharmaceutically acceptable salt, stereoisomer, or tautomer thereof, wherein:
d, m, n, q, w, Q, Ring B, R¹, R², p, Z, Y, Ring A, and T are as detailed herein.

In another aspect, provided herein is a pharmaceutical composition comprising a compound of Formula (Ia), or a pharmaceutically acceptable salt, stereoisomer, or tautomer thereof, and a pharmaceutically acceptable carrier or excipient.

In another aspect, provided herein is a method of making a compound of Formula (Ia), or a pharmaceutically acceptable salt, stereoisomer, or tautomer thereof.

In another aspect, provided herein is a method of inhibiting RIPK3 comprising contacting the kinase with a compound of Formula (Ia), or a pharmaceutically acceptable salt, stereoisomer, or tautomer thereof, or a pharmaceutical composition described herein. Also provided is a method of reducing necroptosis in an individual, comprising administering to the individual a compound of Formula (Ia), or a pharmaceutically acceptable salt, stereoisomer, or tautomer thereof.

### DETAILED DESCRIPTION

The following description is presented to enable a person of ordinary skill in the art to make and use the various embodiments. Descriptions of specific devices, techniques, and applications are provided only as examples. Thus, the various embodiments are not intended to be limited to the examples described herein and shown, but are to be accorded the scope consistent with the claims.

### I. Definitions

The singular forms “a” and “the” include plural references unless the context clearly dictates otherwise.

“Alkyl” refers to an unbranched or branched saturated hydrocarbon chain. As used herein, alkyl may have 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), 1 to 4 carbon atoms (i.e., C₁₋₄ alkyl) or 1 or 3 carbon atoms (i.e., C₁₋₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂). “Alkylene” refers to a bivalent alkyl group.

“Alkoxy” refers to the group “alkyl-O—”. As used herein, alkoxy may have 1 to 20 carbon atoms (i.e., C₁₋₂₀ alkoxy), 1 to 8 carbon atoms (i.e., C₁₋₈ alkoxy), 1 to 6 carbon atoms (i.e., C₁₋₆ alkoxy), 1 to 4 carbon atoms (i.e., C₁₋₄ alkoxy) or 1 or 3 carbon atoms (i.e., C₁₋₃ alkoxy). Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

The term “cycloalkyl”, as used herein, refers to a non-aromatic (e.g., saturated or partially unsaturated) carbocyclic ring moiety. The term “cycloalkyl” encompasses monocyclic ring moieties and polycyclic ring moieties, wherein the polycyclic moieties may be fused, bridged, or spiro. Cycloalkyl includes cycloalkenyl groups, wherein the ring moiety comprises at least one annular double bond. Cycloalkyl includes any polycyclic carbocyclic ring moiety comprising at least one non-aromatic carbocyclic ring, regardless of the point of attachment to the remainder of the molecule. As used herein, cycloalkyl includes rings having, for example, 3 to 20 annular carbon atoms (i.e., a C₃₋₂₀ cycloalkyl), 3 to 14 annular carbon atoms (i.e., C₃₋₁₄ cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀ cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₅cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, bicyclo [2.2.1]heptanyl, and the like. Still further, cycloalkyl also includes spiro cycloalkyl ring moieties, for example, spiro[2.5]octanyl, spiro[4.5]decanyl, or spiro [5.5]undecanyl.

The term “aryl”, as used herein, refers to an aromatic (e.g., fully unsaturated) carbocyclic ring moiety. The term “aryl” encompasses monocyclic ring moieties and polycyclic fused-ring moieties. As used herein, aryl encompasses ring moieties having, for example, 6 to 20 annular carbon atoms (i.e., C₆₋₂₀ aryl), 6 to 16 annular carbon atoms (i.e., C₆₋₁₆ aryl), 6 to 14 annular carbon atoms (i.e., C₆₋₁₄ aryl), 6 to 12 annular carbon atoms (i.e., C₆₋₁₂ aryl), or 6 to 10 annular carbon atoms (i.e., C₆₋₁₀ aryl). Examples of aryl moieties include, but are not limited to, phenyl, naphthyl, and anthryl.

The term “heteroaryl”, as used herein, refers to an aromatic (e.g., fully unsaturated) ring moiety that has one or more (e.g., 1, 2, 3, 4, or 5) annular heteroatoms independently selected from the group consisting of nitrogen, oxygen, and sulfur. The term “heteroaryl” includes both monocyclic ring moieties and polycyclic fused-ring moieties. As used herein, a heteroaryl may have, for example, 5 to 20 annular atoms (i.e., a 5- to 20-membered heteroaryl), 5 to 16 annular atoms (i.e., a 5- to 16-membered heteroaryl), 5 to 14 annular atoms (i.e., a 5- to 14-membered heteroaryl), 5 to 12 annular atoms (i.e., a 5- to 12-membered heteroaryl), 5 to 10 annular atoms (i.e., a 5- to 10-membered heteroaryl), 5 to 8 annular atoms (i.e., a 5- to 8-membered heteroaryl), or 5 to 6 annular atoms (i.e., a 5- to 6-membered heteroaryl), each independently having 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, benzo[d][1,2,3]triazolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thiophenyl (i.e., thienyl), triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one annular heteroatom, is considered a heteroaryl regardless of the attachment to the remainder of the molecule (i.e., through any one of the fused rings). Heteroaryl does not encompass or overlap with aryl as defined above.

The term “heterocyclyl”, as used herein, refers to a non-aromatic (e.g., saturated or partially unsaturated) cyclic moiety that has one or more (e.g., 1, 2, 3, 4, or 5) annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety). Further, the term heterocyclyl is intended to encompass any polycyclic ring moiety comprising at least one annular heteroatom wherein the polycyclic ring moiety comprises at least one non-aromatic ring having an annular heteroatom, regardless of the point of attachment to the remainder of the molecule. As used herein, a heterocyclyl may have, for example, 3 to 20 annular atoms (i.e., a 3- to 20-membered heterocyclyl), 3 to 16 annular atoms (i.e., a 3- to 16-membered heterocyclyl), 3 to 14 annular atoms (i.e., a 3-14 membered heterocyclyl), 3 to 12 annular atoms (i.e., a 3- to 12-membered heterocyclyl), 3 to 10 annular atoms (i.e., a 3- to 10-membered heterocyclyl), 3 to 8 annular atoms (i.e., a 3- to 8-membered heterocyclyl), 3 to 6 annular atoms (i.e., a 3- to 6-membered heterocyclyl), 3 to 5 annular atoms (i.e., a 3- to 5-membered heterocyclyl), 5 to 8 annular atoms (i.e., a 5- to 8-membered heterocyclyl), or 5 to 6 annular atoms (i.e., a 5- to 6-membered heterocyclyl), each independently having 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, dioxolanyl, dihydropyranyl, hydropyranyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, and thiamorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, 6-oxa-1-azaspiro[3.3]heptanyl, and 6-azaspiro[2.5]octanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, isoindolinyl, and azabicyclo[3.1.0]hexanyl, where the heterocyclyl can be bound via either ring of the fused system.

“Halogen” or “halo” includes fluoro, chloro, bromo, and iodo.

“Oxo” refers to the moiety ═O.

The terms “optional” or “optionally” means that the subsequently described event or circumstance may or may not occur. The term “optionally substituted” refers to any one or more hydrogen atoms on the designated atom or group may or may not be replaced by a moiety other than hydrogen. “Optionally substituted” unless otherwise specified means that a group may be unsubstituted or substituted by one or more (e.g., 1, 2, 3, 4 or 5) of the substituents listed for that group in which the substituents may be the same or different.

In one embodiment, an optionally substituted group has one substituent. In another embodiment, an optionally substituted group has two substituents. In another embodiment, an optionally substituted group has three substituents. In some embodiments, an optionally substituted group has more than one substituents, wherein each substituent is independently selected. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted. The substituents may be the same or different. Where there are multiple substituents within a moiety or compound, it is to be understood that each substituent may be selected independently of each other substituent.

As used herein, “treatment” or “treating” is an approach for obtaining a beneficial or desired result, such as a clinical result. For purposes of this disclosure, beneficial or desired results include, but are not limited to, alleviation of a symptom and/or diminishment of the extent of a symptom and/or preventing a worsening of a symptom associated with a disease or condition.

In some embodiments, “an individual” or “a subject” as used herein intends a mammal, including but not limited to a primate, human, bovine, horse, feline, canine, or rodent. In one variation, the individual or subject is a human.

In one variation, beneficial or desired clinical results include, but are not limited to, alleviation of a symptom and/or diminishment of the extent of a symptom and/or preventing a worsening of a symptom associated with a disease mediated by inhibition of RIPK3. Preferably, treatment of a disease or condition with a compound of the disclosure, or a pharmaceutically acceptable salt, stereoisomer, or tautomer thereof, is accompanied by no or fewer side effects than are associated with currently available therapies for the disease or condition and/or improves the quality of life of the individual.

The term “effective amount” as used herein, refers to a sufficient amount of at least one agent being administered to achieve a desired result, e.g., to modulate (e.g., inhibit) a receptor interacting protein kinases such as RIPK3 or to relieve to some extent one or more symptoms of a disease or condition being treated. In certain instances, the method is in vitro, and the desired result may comprise certain desired alteration of cells or biological processes. In certain instances, the method is in vivo, and the result may comprise a reduction and/or alleviation of the signs, symptoms, or causes of a disease, such as a disease mediated by inhibition of RIPK3. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

In certain instances, an “effective amount” is considered in the context of therapeutical uses and may be optionally referred to as “therapeutically effective amount”. A “therapeutically effective amount” refers to an amount of the compound or the composition comprising a compound or salt thereof as set forth herein sufficient to produce a desired therapeutic outcome and/or required to provide a clinically significant decrease in a disease. In various embodiments, an effective amount or a therapeutically effective amount of the compound may modulate (e.g., inhibit) a receptor interacting protein kinase such as RIPK3 or relieve to some extent one or more symptoms of a RIPK3-associated disease or condition.

As used herein, “pharmaceutically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“The term “pharmaceutically acceptable salt”, as used herein, of a given compound refers to salts that retain at least a portion of the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like. Pharmaceutically acceptable salts can be prepared in situ in the manufacturing process, or by separately reacting a purified compound of the disclosure in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (directly compressible), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another and “diastereomers,” which refers to stereoisomers that have at least two asymmetric atoms, but which are not mirror-images of each other.

It is understood that aspects and embodiments described herein as “comprising” include “consisting of” and “consisting essentially of” embodiments.

### II. Compounds of Formula (Ia)

In one aspect, provided herein is a compound of Formula (Ia):

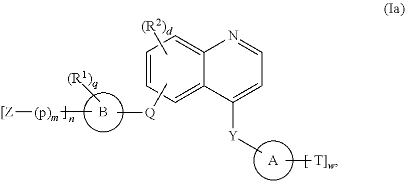

or a pharmaceutically acceptable salt, stereoisomer, or tautomer thereof, wherein: d, m, n, q, w, Q, Ring B, R¹, R², p, Z, Y, Ring A, and T are as detailed herein.

Also provided are stereoisomers, mixture of stereoisomers, tautomers, isotopically enriched analogs, and pharmaceutically acceptable salts of the compounds described herein, such as compounds of Formula (Ia), (I), (II-1), (II-2), (III-1), (III-2), (IV-1), or (IV-2), or variations thereof described herein.

Substituents for rings described herein (e.g. cycloalkyl, heterocyclyl, aryl, heteroaryl) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings) unless context clearly intends otherwise. When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different or the same. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

In the descriptions herein (e.g., Formula (Ia), (I), (II-1), (II-2), (III-1), (III-2), (IV-1), or (IV-2)), it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of Formula (Ia) may be combined with every description, variation, embodiment or aspect of R² of Formula (Ia) the same as if each and every combination were specifically and individually listed and such combinations are equally applicable to other formulae where permitted by the chemical structure.

The compounds disclosed herein, or their pharmaceutically acceptable salts, may include an asymmetric center and may thus give rise to enantiomers, diastereomers, and other stereoisomeric forms that may be defined, in terms of absolute stereochemistry, as (R)- or (S)-. The present disclosure is meant to include all such possible isomers, as well as their racemic and optically pure forms. Optically active (+) and (−), or (R)- and (S)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. In some embodiments, the compound of Formula (Ia) provided herein also encompasses stereoisomers of Formula (Ia) or variations thereof. Thus, it is understood that isomers, such as stereoisomers, of a compound of Formula (Ia), (I), (II-1), (II-2), (III-1), (III-2), (IV-1), or (IV-2), are provided herein.

The present disclosure also encompasses tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Also provided herein are isotopically labeled forms of the compounds, such as a compound of Formula (Ia), (I), (II-1), (II-2), (III-1), (III-2), (IV-1), or (IV-2). These forms of compounds may also be referred to as an “isotopically enriched analog.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Such compounds may exhibit increased resistance to metabolism and may thus be useful for increasing the half-life of any compound when administered to a mammal, particularly a human. Such compounds are synthesized by means well known in the art, for example by employing starting materials in which one or more hydrogens have been replaced by deuterium.

Certain compounds disclosed herein contain one or more ionizable groups (groups from which a proton can be removed (e.g., —COOH) or added (e.g., amines) or which can be quaternized (e.g., amines)). All possible ionic forms of such molecules and salts thereof are intended to be included individually in the disclosure herein. With regard to salts of the compounds described herein, one of ordinary skill in the art can select from among a wide variety of available counterions those that are appropriate.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vitro or in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding.

In some aspects, provided herein is a compound of Formula (Ia):

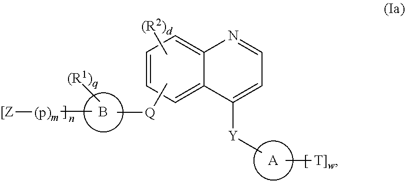

or a pharmaceutically acceptable salt, stereoisomer, or tautomer thereof, wherein:
d is an integer from 0 to 3;
m is an integer from 0 to 2;
n is an integer from 0 to 5;
q is an integer from 0 to 4;
w is an integer from 0 to 9;
Q is a bond or —NH—;
Ring B is C₆₋₁₄ aryl or pyridyl;
each R¹ is independently halo, —CN, C₁₋₆ alkyl or C₁₋₆ alkoxy;
each R² is independently halo;
each p is independently selected from the group consisting of —O—, C₁₋₆ alkylene, —NR^{a}—, —C(O)—, #—NR^{a}SO₂—, #—R^{a}OR^{b}—, #—C(O)NR^{a}—, #—C(O)NR^{a}R^{b}—, #—C(O)R^{a}—, #—C(O)OR^{a}, and #—OR^{a}—, wherein #indicates the point of attachment to Ring B;
each of R^{a} and R^{b} is independently H, C₁₋₆ alkyl, C₁₋₆ alkylene, or —C(O)C₁₋₆ alkyl;
each Z is independently —NR^{c}R^{d}, 3- to 12-membered heterocyclyl optionally substituted with one or more R^{Za}, 5- to 12-membered heteroaryl optionally substituted with one or more R^{Zb}, C₆₋₁₄ aryl optionally substituted with one or more R^{Zc}, or 3- to 12-membered cycloalkyl optionally substituted with one or more R^{Zd};
each R^{Za}, R^{Zb}, R^{Zc}, and R^{Zd} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, —SO₂R^{c}—, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, R^{Zc}, and R^{Zd} is optionally substituted with one or more halo, —OH, C₁₋₆ alkyl, —C(O)OR^{c}, or —C(O)NR^{c}R^{d};
each R^{c} and R^{d} is independently H, —NH₂, or C₁₋₆ alkyl optionally substituted with one or more —OH, halo, —NH₂, —NHC₁₋₆ alkyl, —CF₃, or oxo;
Y is —NH— or —O—;
Ring A is selected from the group consisting of:

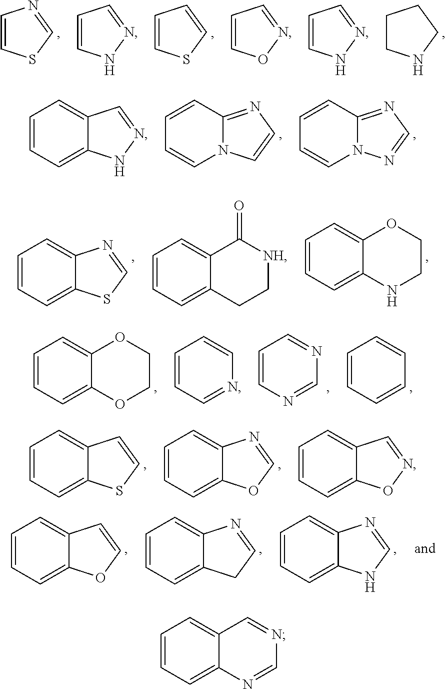

and
each T is independently selected from the group consisting of oxo, halo, —NH₂, C₁-C₆ alkyl, C₁₋₆ alkoxy, —NHC(O)—C₃₋₆cycloalkyl, and methylpiperidyl,
   provided that:
(1) n+w is an integer greater than 0,
(2) when Ring A is phenyl, then either: (a) n is an integer from 1 to 5 and Ring B is substituted by at least one (p)ₘ-Z wherein Z is 3- to 12-membered heterocyclyl substituted with one or more R^{Za} or (b) Ring A is substituted by at least one T wherein T is methylpiperidyl, and
(3) when Ring B is phenyl, m is 0, and Z is piperazinyl, at least one R^{Za} is present and is CH₃ or oxo.

In some aspects, provided herein is a compound of Formula (I):

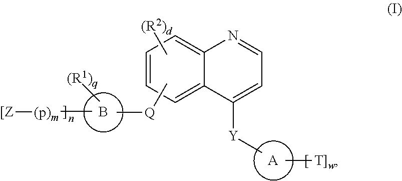

or a pharmaceutically acceptable salt, stereoisomer, or tautomer thereof, wherein:
d is an integer from 0 to 3;
m is an integer from 0 to 2;
n is an integer from 0 to 5;
q is an integer from 0 to 4;
w is an integer from 0 to 9;
Q is a bond or —NH—;
Ring B is C₆₋₁₄ aryl or pyridyl;
each R¹ is independently halo, —CN, C₁₋₆ alkyl or C₁₋₆ alkoxy;
each R² is independently halo;
each p is independently selected from the group consisting of —O—, C₁₋₆ alkylene, —NR^{a}—, —C(O)—, #—NR^{a}SO₂—, #—R^{a}OR^{b}—, #—C(O)NR^{a}R^{b}—, #—C(O)R^{a}—, #—C(O)OR^{a}, and #—OR^{a}—, wherein #indicates the point of attachment to Ring B;
each of R^{a} and R^{b} is independently H, C₁₋₆ alkyl or C₁₋₆ alkylene;
each Z is independently —NR^{c}R^{d}, 3- to 12-membered heterocyclyl optionally substituted with one or more R^{Za}, 5- to 12-membered heteroaryl optionally substituted with one or more R^{Zb}, or C₆₋₁₄ aryl optionally substituted with one or more R^{Zc}; each R^{Za}, R^{Zb}, and R^{Zc} is independently selected from the group consisting of oxo, halo, —OH, O⁻, —NR^{a}R^{b}, C₁₋₆ alkyl, C₁₋₆alkoxy, —C(O)OR^{c}, —C(O)R⁰, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, and R^{Zc} is optionally substituted with one or more halo, —OH, C₁₋₆ alkyl, —C(O)OR^{c}, or —C(O)NR^{c}R^{d};
each R^{c} and R^{d} is independently H or C₁₋₆ alkyl optionally substituted with one or more —OH, halo, —NH₂, —CF₃, or oxo;
Y is —NH— or —O—;

Ring A is selected from the group consisting of:

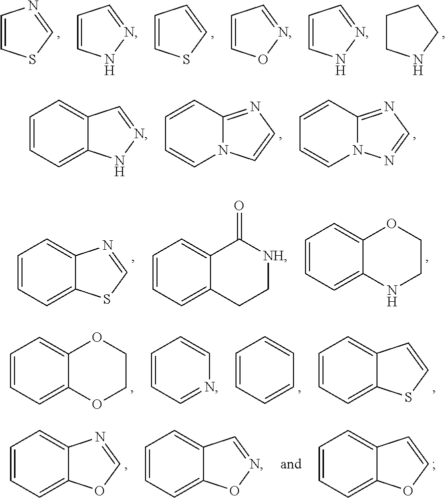

and
each T is independently selected from the group consisting of halo, —NH₂, C₁-C₆ alkyl, C₁₋₆ alkoxy, —NHC(O)—C₃₋₆cycloalkyl, and methylpiperidyl, provided that:
(1) n+w is an integer greater than 0, and
(2) when Ring A is phenyl, then either: (a) n is an integer from 1 to 5 and Ring B is substituted by at least one (p)ₘ-Z wherein Z is 3- to 12-membered heterocyclyl substituted with one or more R^{Za} or (b) Ring A is substituted by at least one T wherein T is methylpiperidyl.

In some embodiments, d is an integer from 0 to 3. In some embodiments, d is 1. In some embodiments, d is 0. In some embodiments, d is 2 or 3. In some embodiments, d is an integer from 1-3.

In some embodiments, m is an integer from 0 to 2. In some embodiments, m is 0. In some embodiments, m is 1. In some embodiments, m is 2. In some embodiments, m is 0 or 1.

In some embodiments, n is an integer from 0 to 5. In some embodiments, n is 0. In some embodiments, n is 1. In some embodiments, n is 0 or 1. In some embodiments, n is 2, 3, 4, or 5.

In some embodiments, q is an integer from 0 to 4. In some embodiments, q is 1. In some embodiments, q is 0. In some embodiments, q is 0 or 1. In some embodiments, q is 2, 3, or 4.

In some embodiments, w is an integer from 0 to 9. In some embodiments, w is 0. In some embodiments, w is 1. In some embodiments, w is 0 or 1. In some embodiments, w is 3. In some embodiments, w is 0, 1, 2, or 3. In some embodiments, w is an integer from 1-3.

In some embodiments, Q is a bond or —NH—. In some embodiments, Q is a bond. In some embodiments, Q is —NH—.

In some embodiments, Ring B is C₆₋₁₄ aryl or pyridyl. In some embodiments, Ring B is C₆₋₁₄ aryl. In some embodiments, Ring B is phenyl. In some embodiments, Ring B is pyridyl.

In some embodiments, each R¹ is independently halo, —CN, C₁₋₆ alkyl or C₁₋₆ alkoxy. In some embodiments, each R¹ is independently halo. In some embodiments, each R¹ is independently fluoro or chloro. In some embodiments, each R¹ is fluoro. In some embodiments, each R¹ is chloro. In some embodiments, each R¹ is —CN. In some embodiments, each R¹ is C₁₋₆ alkyl. In some embodiments, each R¹ is methyl. In some embodiments, each R¹ is C₁₋₆ alkoxy. In some embodiments, each R¹ is methoxy. In some embodiments, q is an integer from 1-4 and at least one R¹ is halo. In some embodiments, q is an integer from 1-4 and at least one R¹ is —CN. In some embodiments, q is an integer from 1-4 and at least one R¹ is C₁₋₆ alkyl. In some embodiments, q is an integer from 1-4 and at least one R¹ is C₁₋₆ alkoxy.

In some embodiments, each R² is independently halo. In some embodiments, each R² is independently fluoro or chloro. In some embodiments, each R² is fluoro. In some embodiments, each R² is chloro.

In some embodiments, each p is independently selected from the group consisting of —O—, C₁₋₆ alkylene, —NR^{a}—, —C(O)—, #—NR^{a}SO₂—, #—R^{a}OR^{b}—, #—C(O)NR^{a}—, #—C(O)NR^{a}R^{b}—, #—C(O)R^{a}—, #—C(O)OR^{a}, and #—OR^{a}—, wherein #indicates the point of attachment to Ring B, wherein each of R^{a} and R^{b} is independently H, C₁₋₆ alkyl, C₁₋₆ alkylene or —C(O)C₁₋₆ alkyl. In some embodiments, each p is independently selected from the group consisting of —O—, —C(O)—, and C₁₋₆ alkylene. In some embodiments, each p is independently selected from the group consisting of —O—, —C(O)—, and methylene. In some embodiments, m is 1, and p is —C(O)—. In some embodiments, m is 1, and p is C₁₋₆ alkylene. In some embodiments, m is 1, and p is methylene. In some embodiments, m is 1, and p is —O—. In some embodiments, m is 1, and p is —NR^{a}—, wherein R^{a} is H, C₁₋₆ alkyl, C₁₋₆ alkylene, or —C(O)C₁₋₆ alkyl. In some embodiments, m is 1, and p is —NR^{a}—, wherein R^{a} is H. In some embodiments, m is 1, and p is #—C(O)NR^{a}R^{b}—, wherein #indicates the point of attachment to Ring B, wherein each of R^{a} and R^{b} is independently H, C₁₋₆ alkyl, C₁₋₆ alkylene, or —C(O)C₁₋₆ alkyl. In some embodiments,
m is 1, and p is #—C(O)NHR^{b}, wherein R^{b} is C₁₋₆ alkylene. In some embodiments, m is 1, and p is #—C(O)OR^{a}, wherein #indicates the point of attachment to Ring B, wherein R^{a} is H, C₁₋₆ alkyl or C₁₋₆ alkylene. In some embodiments, m is 1, and p is #—NR^{a}SO₂—, wherein #indicates the point of attachment to Ring B, wherein R^{a} is H, C₁₋₆ alkyl or C₁₋₆ alkylene. In some embodiments, m is 1, and p is #—NHSO₂—, wherein #indicates the point of attachment to Ring B. In some embodiments, m is 2, and each p is independently selected from the group consisting of —C(O)— and —NR^{a}—, wherein R^{a} is H, C₁₋₆ alkyl or C₁₋₆ alkylene. In some embodiments, m is 2, and each p is independently selected from the group consisting of —C(O)— and —NH—. In some embodiments, m is 2, and each p is independently selected from the group consisting of —O— and C₁₋₆ alkylene. In some embodiments, m is 2, and each p is independently selected from the group consisting of —O— and ethylene.

In some embodiments, each p is independently selected from the group consisting of —O—, C₁₋₆ alkylene, —NR^{a}—, —C(O)—, #—NR^{a}SO₂—, #—R^{a}OR^{b}—, #—C(O)NR^{a}R^{b}—, #—C(O)R^{a}_, #—C(O)OR^{a}, and #—OR^{a}—, wherein #indicates the point of attachment to Ring B, wherein each of R^{a} and R^{b} is independently H, C₁₋₆ alkyl or C₁₋₆ alkylene. In some embodiments, each p is independently selected from the group consisting of —O—, —C(O)—, and C₁₋₆ alkylene. In some embodiments, each p is independently selected from the group consisting of —O—, —C(O)—, and methylene. In some embodiments, m is 1, and p is —C(O)—. In some embodiments, m is 1, and p is C₁₋₆ alkylene. In some embodiments, m is 1, and p is methylene. In some embodiments, m is 1, and p is —O—. In some embodiments, m is 1, and p is —NR^{a}—, wherein R^{a} is H, C₁₋₆ alkyl or C₁₋₆ alkylene. In some embodiments, m is 1, and p is —NR^{a}—, wherein R^{a} is H. In some embodiments, m is 1, and p is #—C(O)NR^{a}R^{b}—, wherein #indicates the point of attachment to Ring B, wherein each of R^{a} and R^{b} is independently H, C₁₋₆ alkyl or C₁₋₆ alkylene. In some embodiments, m is 1, and p is #—C(O)NHR^{b}, wherein R^{b} is C₁₋₆ alkylene. In some embodiments, m is 1, and p is #—C(O)OR^{a}, wherein #indicates the point of attachment to Ring B, wherein R^{a} is H, C₁₋₆ alkyl or C₁₋₆ alkylene. In some embodiments, m is 1, and p is #—NR^{a}SO₂—, wherein #indicates the point of attachment to Ring B, wherein R^{a} is H, C₁₋₆ alkyl or C₁₋₆ alkylene. In some embodiments, m is 1, and p is #—NHSO₂—, wherein #indicates the point of attachment to Ring B. In some embodiments, m is 2, and each p is independently selected from the group consisting of —C(O)— and —NR^{a}—, wherein R^{a} is H, C₁₋₆ alkyl or C₁₋₆ alkylene. In some embodiments, m is 2, and each p is independently selected from the group consisting of —C(O)— and —NH—. In some embodiments, m is 2, and each p is independently selected from the group consisting of —O— and C₁₋₆ alkylene. In some embodiments, m is 2, and each p is independently selected from the group consisting of —O— and ethylene.

In some embodiments, each Z is independently —NR^{c}R^{d}, 3- to 12-membered heterocyclyl optionally substituted with one or more R^{Za}, 5- to 12-membered heteroaryl optionally substituted with one or more R^{Zb}, C₆₋₁₄ aryl optionally substituted with one or more R^{Zc}, or 3- to 12-membered cycloalkyl optionally substituted with one or more R^{Zd}, wherein each R^{Za}, R^{Zb}, R^{Zc}, and R^{Zd} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, —SO₂R^{c}—, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, R^{Zc}, and R^{Zd} is optionally substituted with one or more halo, —OH, C₁₋₆ alkyl, —C(O)OR^{c}, or —C(O)NR^{c}R^{d}, and wherein each R^{c} and R^{d} is independently H or C₁₋₆ alkyl optionally substituted with one or more —OH, halo, —NH₂, —CF₃, or oxo. In some embodiments, each Z is independently —NR^{c}R^{d}, wherein each R^{c} and R^{d} is independently H, —NH₂, or C₁₋₆ alkyl optionally substituted with one or more —OH, halo, —NH₂, —CF₃, or oxo.

In some embodiments, each Z is independently —NR^{c}R^{d}, 3- to 12-membered heterocyclyl optionally substituted with one or more R^{Za}, 5- to 12-membered heteroaryl optionally substituted with one or more R^{Zb}, or C₆₋₁₄ aryl optionally substituted with one or more R^{Zc}, wherein each R^{Za}, R^{Zb}, and R^{Zc} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, and R^{Zc} is optionally substituted with one or more halo, —OH, C₁₋₆ alkyl, —C(O)OR^{c}, or —C(O)NR^{c}R^{d}, and wherein each R^{c} and R^{d} is independently H or C₁₋₆ alkyl optionally substituted with one or more —OH, halo, —NH₂, —CF₃, or oxo. In some embodiments, each Z is independently —NR^{c}R^{d}, wherein each R^{c} and R^{d} is independently H or C₁₋₆ alkyl optionally substituted with one or more —OH, halo, —NH₂, —CF₃, or oxo.

In some embodiments, each Z is independently 3- to 12-membered heterocyclyl optionally substituted with one or more R^{Za}, wherein each R^{Za} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, —SO₂R^{c}—, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, and wherein each R^{c} and R^{d} is independently H, —NH₂, or C₁₋₆ alkyl optionally substituted with one or more —OH, halo, —NH₂, —CF₃, or oxo. In some embodiments, each Z is independently 3- to 12-membered heterocyclyl. In some embodiments, each Z is independently 3- to 12-membered heterocyclyl optionally substituted with one or more R^{Za}, wherein each R^{Za} is independently selected from the group consisting of oxo, halo, and C₁₋₆ alkyl. In some embodiments, each Z is independently 3- to 12-membered heterocyclyl optionally substituted with one or more R^{Za}, wherein each R^{Za} is independently C₁₋₆ alkyl.

In some embodiments, each Z is independently 3- to 12-membered heterocyclyl optionally substituted with one or more R^{Za}, wherein each R^{Za} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, and wherein each R^{c} and R^{d} is independently H or C₁₋₆ alkyl optionally substituted with one or more —OH, halo, —NH₂, —CF₃, or oxo. In some embodiments, each Z is independently 3- to 12-membered heterocyclyl. In some embodiments, each Z is independently 3- to 12-membered heterocyclyl optionally substituted with one or more R^{Za}, wherein each R^{Za} is independently selected from the group consisting of oxo, halo, and C₁₋₆ alkyl. In some embodiments, each Z is independently 3- to 12-membered heterocyclyl optionally substituted with one or more R^{Za}, wherein each R^{Za} is independently C₁₋₆ alkyl.

In some embodiments, each Z is independently piperidinyl, piperazinyl, pyrrolidinyl, or morpholinyl. In some embodiments, each Z is independently piperidinyl, piperazinyl, pyrrolidinyl, or morpholinyl, each of which is optionally substituted with one or more R^{Za}, wherein each R^{Za} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, and wherein each R^{c} and R^{d} is independently H or C₁₋₆ alkyl optionally substituted with one or more —OH, halo, —NH₂, —CF₃, or oxo. In some embodiments, each Z is piperidinyl, piperazinyl, pyrrolidinyl, or morpholinyl, each of which is optionally substituted with one or more R^{Za}, wherein each R^{Za} is independently selected from the group consisting of oxo, halo, and C₁₋₆ alkyl. In some embodiments, each Z is piperidinyl or piperazinyl, each of which is optionally substituted with one or more R^{Za}, wherein each R^{Za} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁. 6alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, and wherein each R^{c} and R^{d} is independently H or C₁₋₆ alkyl optionally substituted with one or more —OH, halo, —NH₂, —CF₃, or oxo. In some embodiments, each Z is piperidinyl or piperazinyl, each of which is optionally substituted with one or more R^{Za}, wherein each R^{Za} is independently selected from the group consisting of oxo, halo, and C₁₋₆ alkyl. In some embodiments, each Z is piperidinyl or piperazinyl, each of which is optionally substituted with one or more R^{Za}, wherein each R^{Za} is independently selected from the group consisting of oxo, halo, and methyl. In some embodiments, each Z is piperidinyl or piperazinyl, each of which is optionally substituted with one or more R^{Za}, wherein each R^{Za} is independently selected from the group consisting of oxo and methyl. In some embodiments, each Z is piperidinyl or piperazinyl, each of which is optionally substituted with one or more R^{Za}, wherein each R^{Za} is C₁₋₆ alkyl.

In some embodiments, each Z is piperidinyl. In some embodiments, each Z is piperidinyl optionally substituted with one or more R^{Za}, wherein each R^{Za} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, wherein each of R^{a} and R^{b} is independently H, C₁₋₆ alkyl or C₁₋₆ alkylene, and wherein each R^{c} and R^{d} is independently H or C₁₋₆ alkyl optionally substituted with one or more —OH, halo, —NH₂, —CF₃, or oxo. In some embodiments, each Z is piperidinyl optionally substituted with one or more R^{Za}, wherein each R^{Za} is independently selected from the group consisting of oxo, halo, and C₁₋₆ alkyl. In some embodiments, each Z is piperidinyl optionally substituted with one or more R^{Za}, wherein each R^{Za} is independently selected from the group consisting of oxo, halo, and methyl. In some embodiments, each Z is piperidinyl optionally substituted with one or more R^{Za}, wherein each R^{Za} is independently selected from the group consisting of oxo and methyl. In some embodiments, each Z is piperidinyl optionally substituted with one or more R^{Za}, wherein each R^{Za} is C₁₋₆ alkyl. In some embodiments, each Z is piperidinyl substituted with one or more R^{Za}, wherein each R^{Za} is methyl.

In some embodiments, each Z is piperazinyl. In some embodiments, each Z is piperazinyl optionally substituted with one or more R^{Za}, wherein each R^{Za} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, wherein each of R^{a} and R^{b} is independently H, C₁₋₆ alkyl or C₁₋₆ alkylene, and wherein each R^{c} and R^{d} is independently H or C₁₋₆ alkyl optionally substituted with one or more —OH, halo, —NH₂, —CF₃, or oxo. In some embodiments, each Z is piperazinyl optionally substituted with one or more R^{Za}, wherein each R^{Za} is independently selected from the group consisting of oxo, halo, and C₁₋₆ alkyl. In some embodiments, each Z is piperazinyl optionally substituted with one or more R^{Za}, wherein each R^{Za} is independently selected from the group consisting of oxo, halo, and methyl. In some embodiments, each Z piperazinyl optionally substituted with one or more R^{Za}, wherein each R^{Za} is independently selected from the group consisting of oxo and methyl. In some embodiments, each Z is piperazinyl optionally substituted with one or more R^{Za}, wherein each R^{Za} is C₁₋₆ alkyl. In some embodiments, each Z is piperazinyl substituted with one or more R^{Za}, wherein each R^{Za} is methyl.

In some embodiments, each Z is independently diazepanyl, dihydropyridinyl, oxa-azaspirononanyl, diazabicyclooctanyl, tetrahydropyranyl, azabicycloheptanyl, imidazolidinyl, azetidinyl, tetrahydropyridinyl, octahydroindolizinyl, or oxetanyl, each of which is optionally substituted with one or more R^{Za}, wherein each R^{Za} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, wherein each of R^{a} and R^{b} is independently H, C₁₋₆ alkyl or C₁₋₆ alkylene, and wherein each R^{c} and R^{d} is independently H or C₁₋₆ alkyl optionally substituted with one or more —OH, halo, —NH₂, —CF₃, or oxo. In some embodiments, each Z is independently 1,4-diazepanyl, 1,2-dihydropyridinyl, 7-oxa-2-azaspiro[3.5]nonyl, 3,8-diazabicyclo[3.2.1]octanyl, tetrahydro-2H-pyranyl, 6-azabicyclo[3.1.1]heptanyl, imidazolidinyl, azetidinyl, 1,2,3,5-tetrahydropyridinyl, octahydroindolizinyl, or oxetanyl, each of which is optionally substituted with one or more R^{Za}, wherein each R^{Za} is independently selected from the group consisting of oxo, halo, and C₁₋₆ alkyl.

In some embodiments, each Z is independently 5- to 12-membered heteroaryl optionally substituted with one or more R^{Zb}, wherein each R^{Zb} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Zb} is optionally substituted with one or more halo, —OH, C₁₋₆ alkyl, —C(O)OR^{c}, or —C(O)NR^{c}R^{d}, wherein each of R^{a} and R^{b} is independently H, C₁₋₆ alkyl or C₁₋₆ alkylene, and wherein each R^{c} and R^{d} is independently H or C₁₋₆ alkyl optionally substituted with one or more —OH, halo, —NH₂, —CF₃, or oxo. In some embodiments, each Z is pyridinyl. In some embodiments, each Z is pyridinyl optionally substituted with one or more R^{Zb}, wherein each R^{Zb} is independently —NR^{a}R^{b}, wherein each of R^{a} and R^{b} is independently H, C₁₋₆ alkyl or C₁₋₆ alkylene. In some embodiments, each Z is thiadiazolyl.

In some embodiments, each Z is independently C₆₋₁₄ aryl optionally substituted with one or more R^{Zc}, wherein each R^{Zc} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Zb} is optionally substituted with one or more halo, —OH, C₁₋₆ alkyl, —C(O)OR^{c}, or —C(O)NR^{c}R^{d}, wherein each of R^{a} and R^{b} is independently H, C₁₋₆ alkyl or C₁₋₆ alkylene, and wherein each R^{c} and R^{d} is independently H or C₁₋₆ alkyl optionally substituted with one or more —OH, halo, —NH₂, —CF₃, or oxo. In some embodiments, each Z is phenyl. In some embodiments, each Z is phenyl optionally substituted with one or more R^{Zc}, wherein each R^{Zc} is independently selected from the group consisting of oxo, halo, and C₁₋₆ alkyl. In some embodiments, each Z is phenyl optionally substituted with one or more R^{Zc}, wherein each R^{Zc} is halo. In some embodiments, each Z is phenyl optionally substituted with one or more R^{Zc}, wherein each R^{Zc} is fluoro.

In some embodiments, each Z is independently 3- to 12-membered cycloalkyl optionally substituted with one or more R^{Zd}, wherein each R^{Zd} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za} is optionally substituted with one or more halo, —OH, C₁₋₆ alkyl, —C(O)OR^{c}, or —C(O)NR^{c}R^{d}, wherein each of R^{a} and R^{b} is independently H, C₁₋₆ alkyl or C₁₋₆ alkylene, and wherein each R^{c} and R^{d} is independently H or C₁₋₆ alkyl optionally substituted with one or more —OH, halo, —NH₂, —CF₃, or oxo. In some embodiments, each Z is 3- to 6-membered cycloalkyl optionally substituted with one or more R^{Zd}. In some embodiments, each Z is cyclobutyl optionally substituted with one or more R^{Zd}. In some embodiments, each Z is cyclobutyl optionally substituted with one or more R^{Zd}, wherein each R^{Zd} is independently selected from the group consisting of oxo, halo, C₁₋₆ alkyl, and C₁₋₆alkoxy, wherein each C₁₋₆ alkyl, and C₁₋₆alkoxy of R^{Zd} is optionally substituted with one or more halo. In some embodiments, each Z is bicyclo[1.1.1]pentanyl, optionally substituted with one or more R^{Zd}. In some embodiments, each Z is bicyclo[1.1.1]pentanyl optionally substituted with one or more R^{Zd}, wherein each R^{Zd} is independently selected from the group consisting of oxo, halo, C₁₋₆ alkyl, and C₁₋₆alkoxy, wherein each C₁₋₆ alkyl, and C₁₋₆alkoxy of R^{Zd} is optionally substituted with one or more halo.

In some embodiments, each Z is independently —NR^{c}R^{d}, wherein each R^{c} and R^{d} is independently H or C₁₋₆ alkyl optionally substituted with one or more —OH, halo, —NH₂, —CF₃, or oxo. In some embodiments, each Z is independently —NR^{c}R^{d}, wherein each R^{c} and R^{d} is C₁. 6 alkyl. In some embodiments, each Z is independently —NR^{c}R^{d}, wherein each R^{c} and R^{d} is methyl. In some embodiments, each Z is independently —NR^{c}R^{d}, wherein each R^{c} and R^{d} is ethyl. In some embodiments, each Z is independently —NR^{c}R^{d}, wherein each R^{c} and R^{d} is independently H or C₁₋₆ alkyl optionally substituted with one or more —OH. In some embodiments, each Z is independently —NR^{c}R^{d}, wherein one of R^{c} and R^{d} is H and the other of R^{c} and R^{d} is methyl, ethyl, propyl, or butyl. In some embodiments, each Z is independently —NR^{c}R^{d}, wherein one of R^{c} and R^{d} is H and the other of R^{c} and R^{d} is methyl, ethyl, propyl, or butyl, each of which is optionally substituted with one or more —OH, halo, or —NH₂.

In some embodiments, n is an integer from 1-5 and at least one Z is —NR^{c}R^{d}. In some embodiments, n is an integer from 1-5 and at least one Z is 3- to 12-membered heterocyclyl optionally substituted with one or more R^{Za}. In some embodiments, Z is substituted with one or more R^{Za} that is C₁₋₆alkyl. In some embodiments, Z is substituted with one or more R^{Za} that is oxo. In some embodiments, n is an integer from 1-5 and Z is 5- to 12-membered heteroaryl optionally substituted with one or more R^{Zb}. In some embodiments, n is an integer from 1-5 and Z is C₆₋₁₄ aryl, optionally substituted with one or more R^{Zc}. In some embodiments, Z is substituted with one or more R^{Zc} that is halo.

In some embodiments, each Z is selected from the group consisting of piperazinyl, piperidinyl, azabicycloheptyl, morpholinyl, azetidinyl, oxetanyl, pyrrolidinyl, imidazolidinyl, octahydroindolizinyl, tetrahydropyridinyl, dihydropyridinyl, diazepanyl, oxa-azaspirononanyl, diazabicyclooctanyl, diazaspiroheptanyl, azaspiroheptanyl, oxa-azaspiroheptanyl, diazaspiroundecanyl, diazaspirodecanyl, diazaspirononanyl, diazabicycloheptanyl, and isoindolinyl, each of which is optionally substituted with one or more R^{Za}; pyridyl and thiadiazolyl, each of which is optionally substituted with one or more R^{Zb}; phenyl optionally substituted with one or more R^{Zc}; and cyclobutyl and bicyclopentane, each of which is optionally substituted with one or more R^{Zd}.

In some embodiments, each Z is selected from the group consisting of

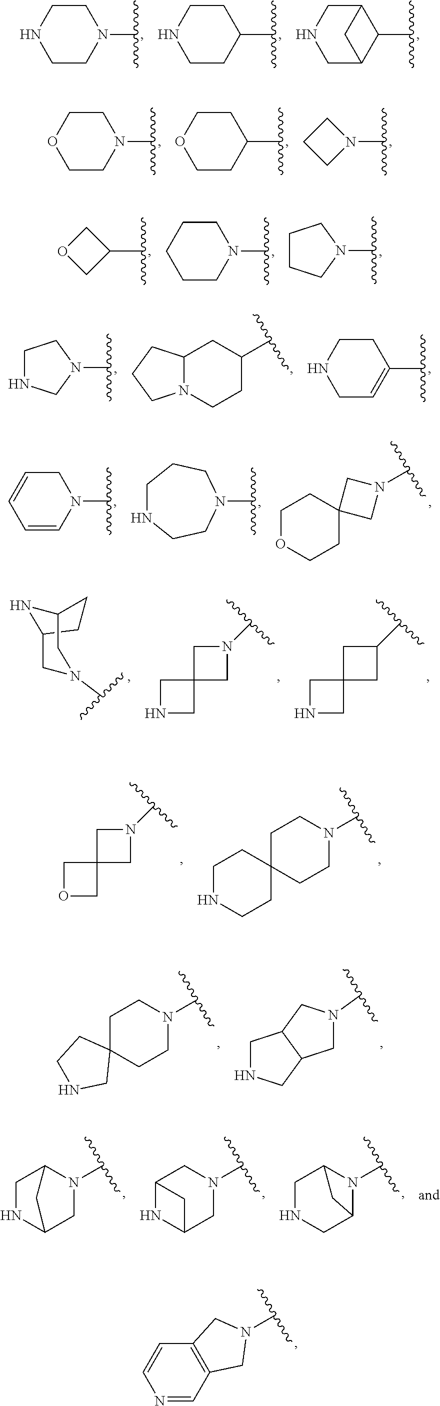

each of which is optionally substituted with one or more R^{Za};

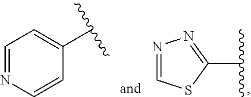 [SMILES: *c1ccncc1 ; *c1nncs1]

each of which is optionally substituted with one or more R^{Zb};

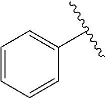 [SMILES: *c1ccccc1]

optionally substituted with one or
more R^{Zc}; and

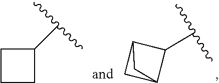 [SMILES: *C1CCC1 ; *C1C2CC1C2]

each of which is optionally substituted with one or more R^{Zd}. In some embodiments, each Z is selected from the group consisting of

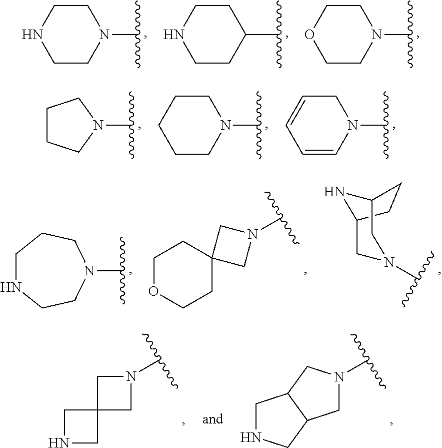

each of which is optionally substituted with one or more R^{Za}.

In some embodiments, each Z is selected from the group consisting of

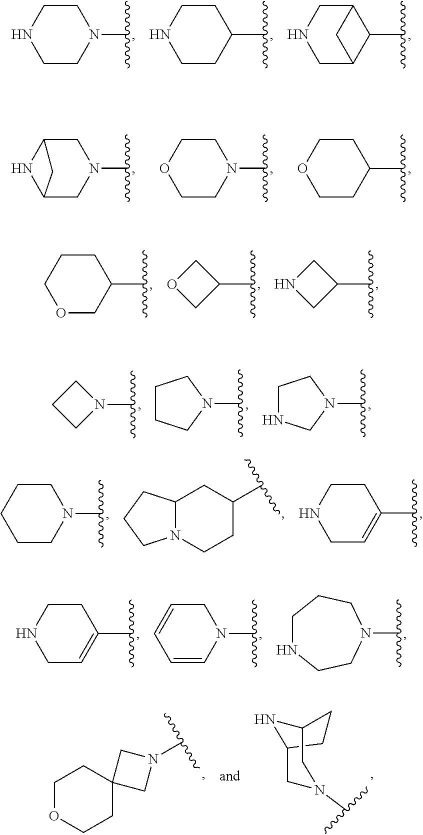

each of which is optionally substituted with one or more R^{Za};

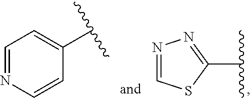 [SMILES: *c1ccncc1 ; *c1nncs1]

each of which is optionally substituted with one or more R^{Zb}; and

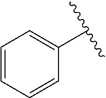 [SMILES: *c1ccccc1]

optionally substituted with one or more R^{Zc}. In some embodiments, each Z is selected from the group consisting of

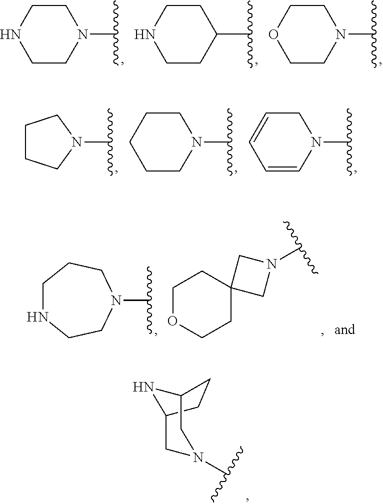

each of which is optionally substituted with one or more R^{Za}

In some embodiments, each R^{Za}, R^{Zb}, and R^{Zc} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, and R^{Zc} is optionally substituted with one or more halo, —OH, C₁₋₆ alkyl, —C(O)OR^{c}, or —C(O)NR^{c}R^{d}. In some embodiments, R^{Za}, R^{Zb}, R^{Zc}, or R^{Zd} is selected from the group consisting of ═O, —O—, —Cl, —F, —OH, —CH₃, —CF₃, NH₂,

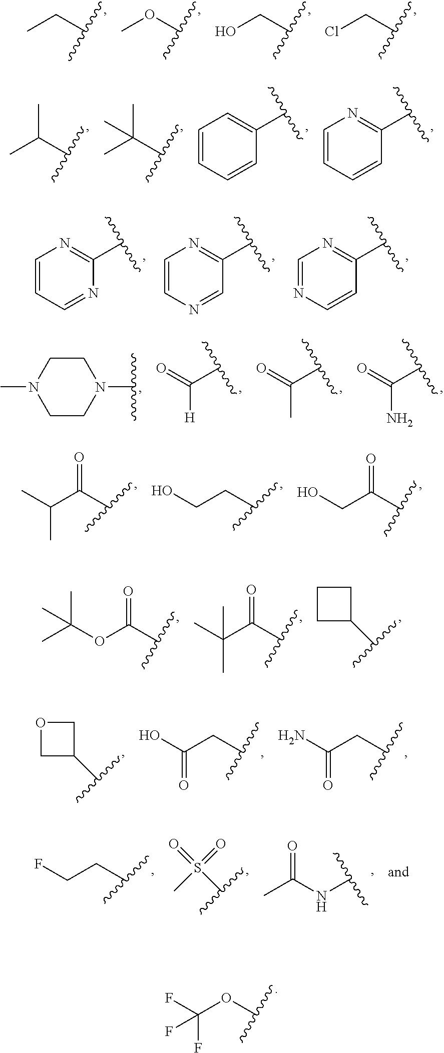

In some embodiments, each R^{Za}, R^{Zb}, or R^{Zc} is independently selected from the group consisting of ═O and —CH₃.

In some embodiments, each R^{Za}, R^{Zb}, and R^{Zc} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, and R^{Zc} is optionally substituted with one or more halo, —OH, C₁₋₆ alkyl, —C(O)OR^{c}, or —C(O)NR^{c}R^{d}. In some embodiments, each R^{Za}, R^{Zb}, or R^{Zc} is independently selected from the group consisting of ═O, —O—, —Cl, —F, —OH, —CH₃, —CF₃, NH₂,

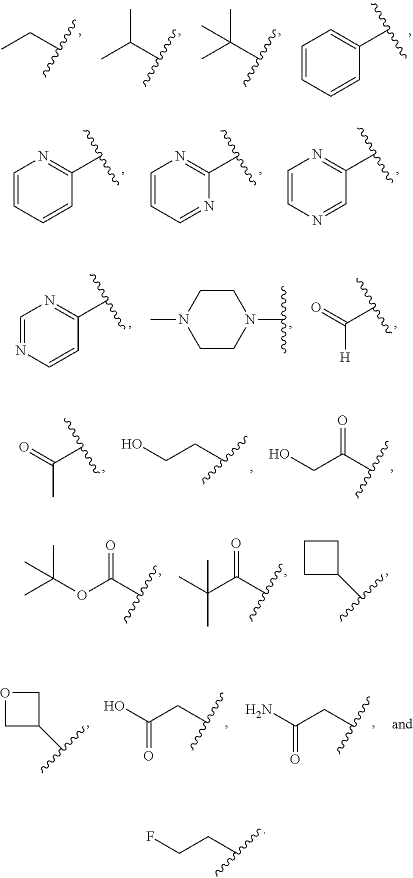

In some embodiments, each R^{Za}, R^{Zb}, or R^{Zc} is independently selected from the group consisting of ═O and —CH₃.

In some embodiments, each Z is independently selected from the group consisting of

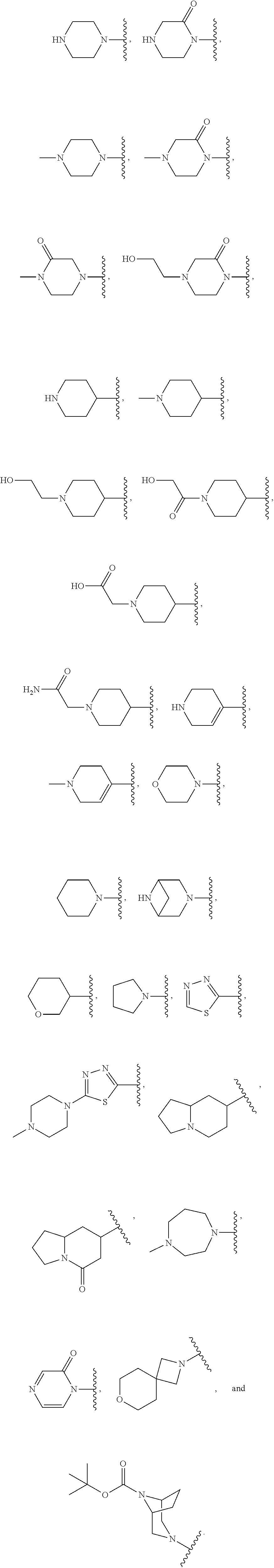

In some embodiments, each Z is independently selected from the group consisting of

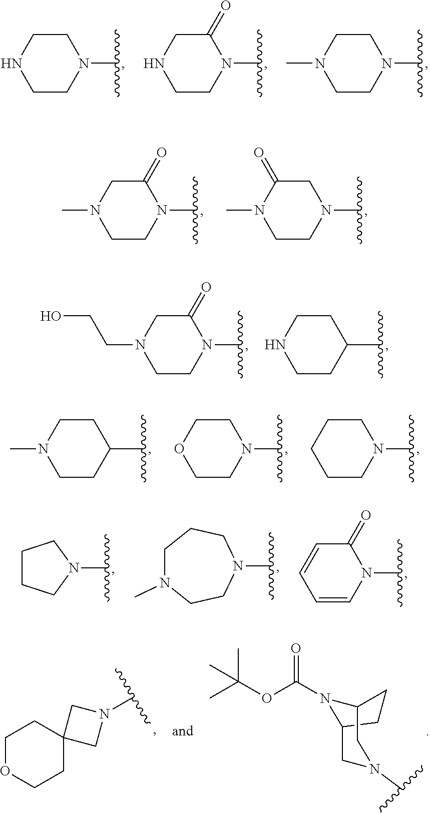

In some embodiments, each Z is independently selected from the group consisting of

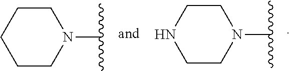 [SMILES: *N1CCCCC1 ; *N1CCNCC1]

In some embodiments each Z-(p)ₘ- is selected from the group consisting of

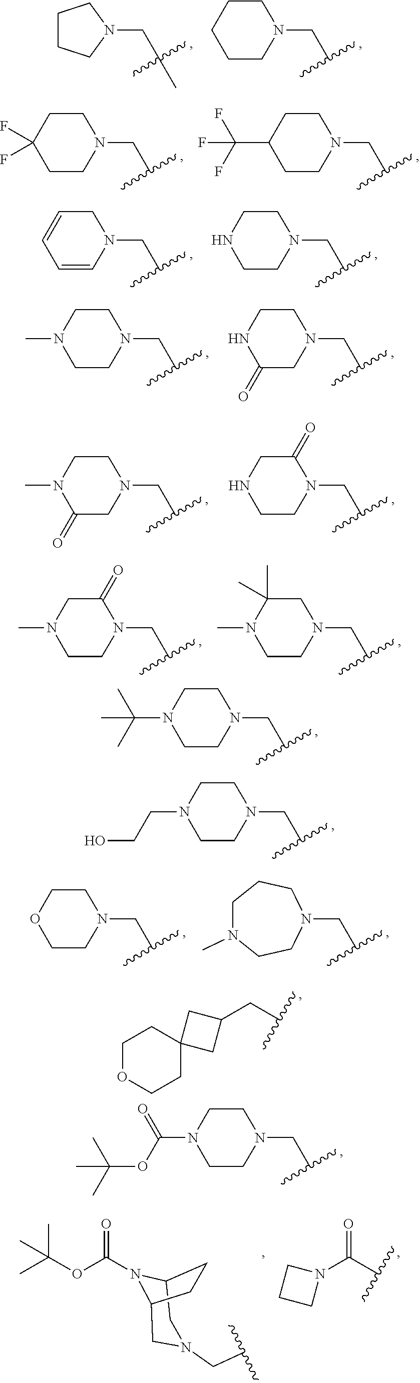

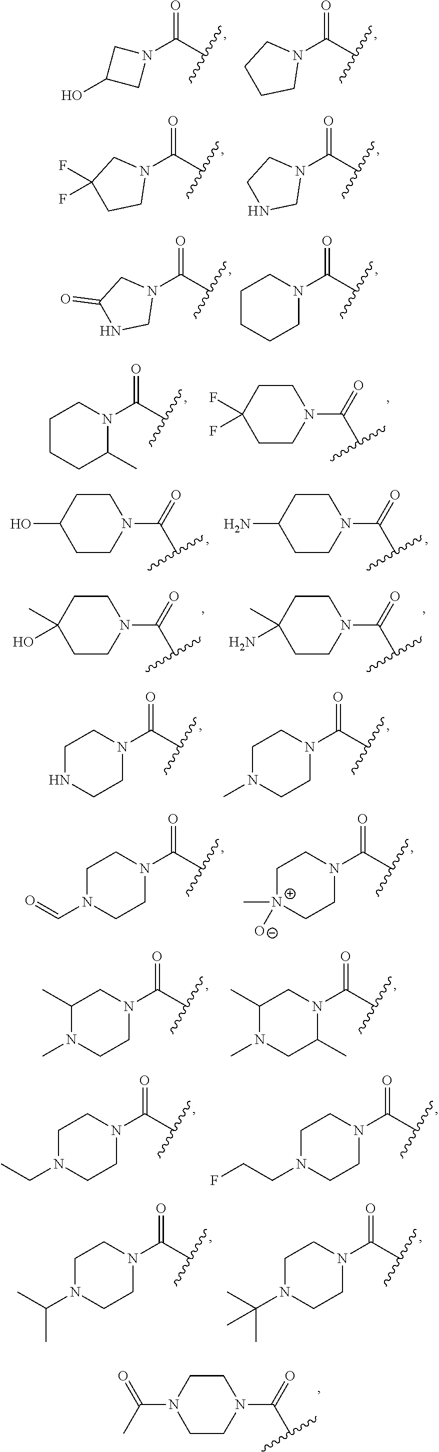

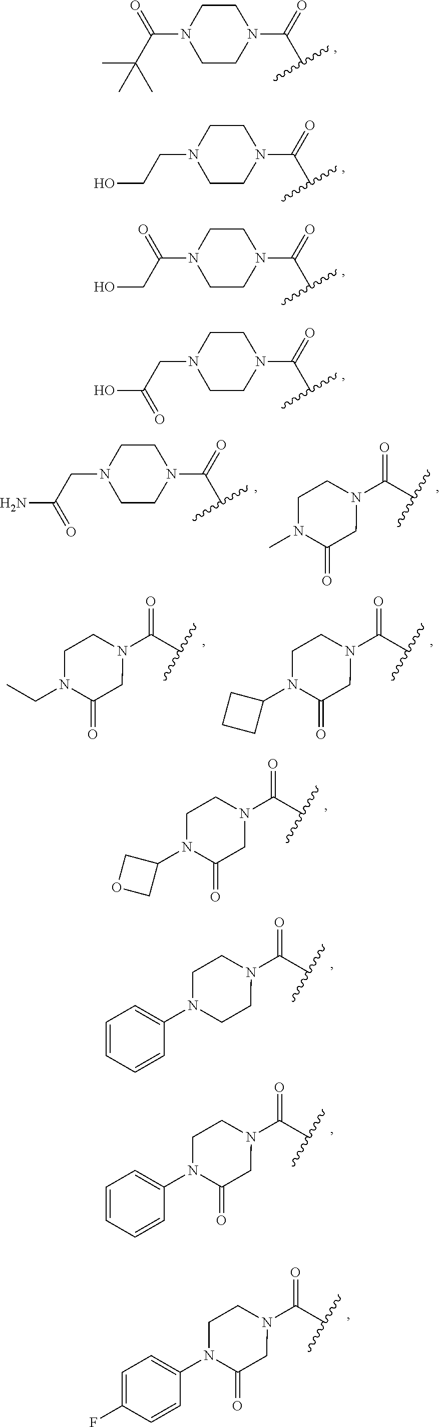

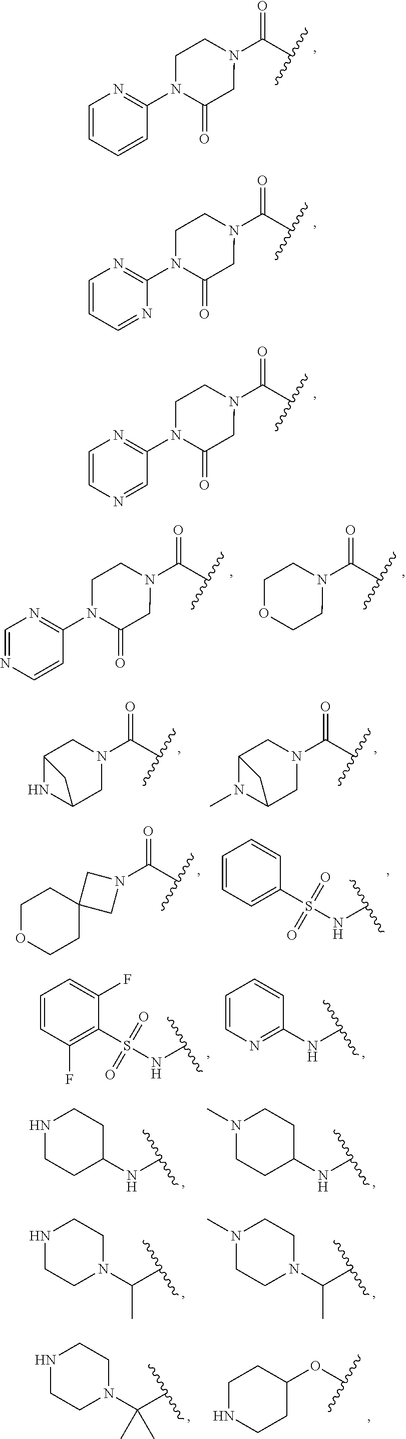

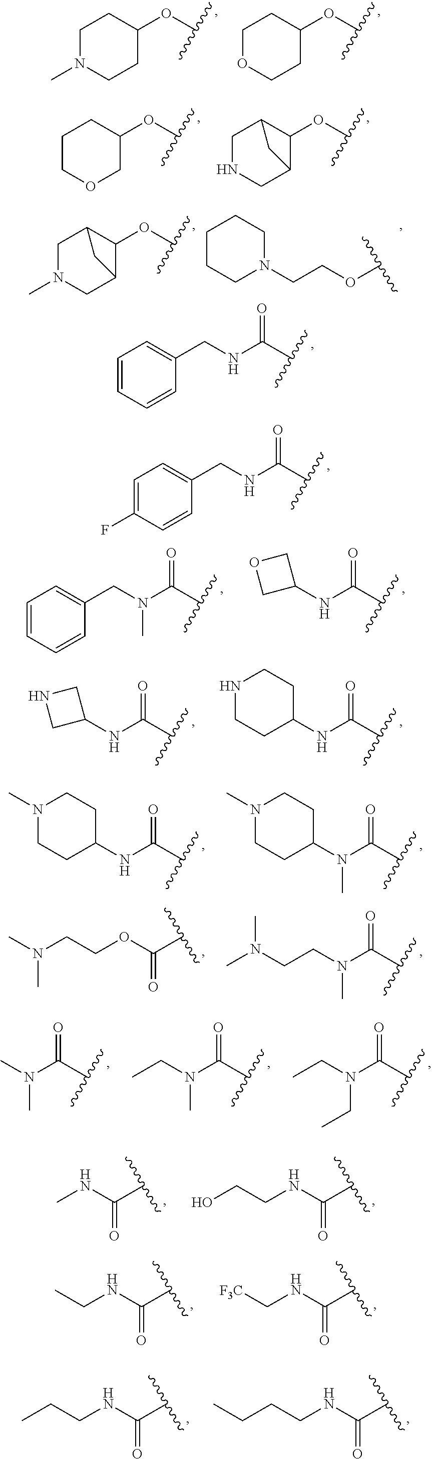

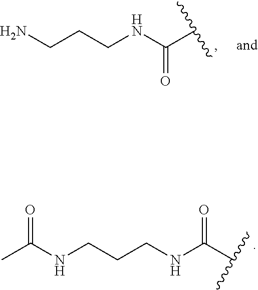 [SMILES: *C(=O)NCCCN ; *C(=O)NCCCNC(C)=O]

In some embodiments, n is 1, and Z-(p)ₘ- is selected from the group consisting of

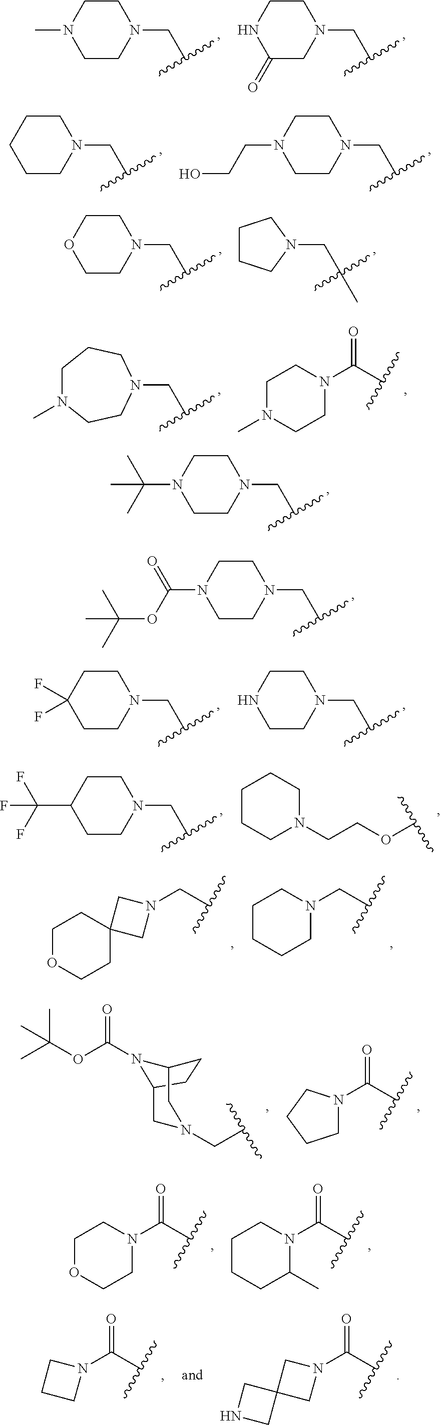

In some embodiments, n is 1, and Z-(p)ₘ- is

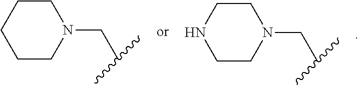 [SMILES: *CN1CCCCC1 ; *CN1CCNCC1]

In some embodiments, n is 1, and Z-(p)ₘ- is selected from the group consisting of

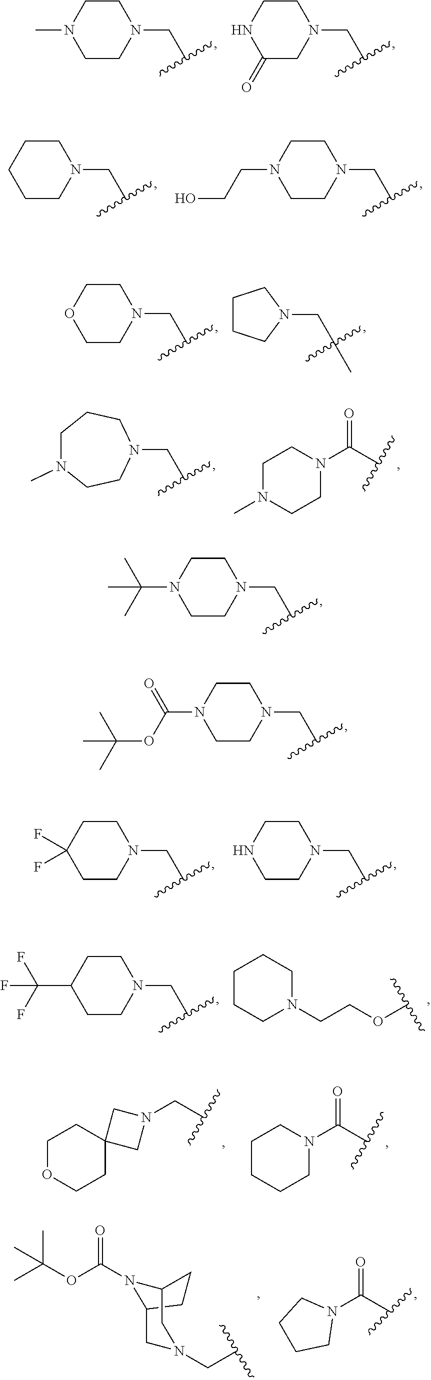

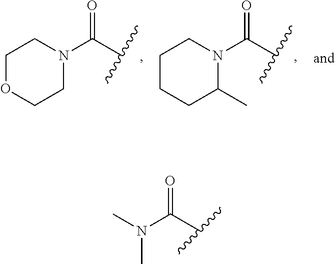

In some embodiments, n is 1, and Z-(p)ₘ- is

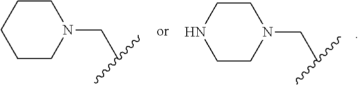 [SMILES: *CN1CCCCC1 ; *CN1CCNCC1]

In some embodiments, n is 1, and Z-(p)ₘ-Ring B is selected from the group consisting of

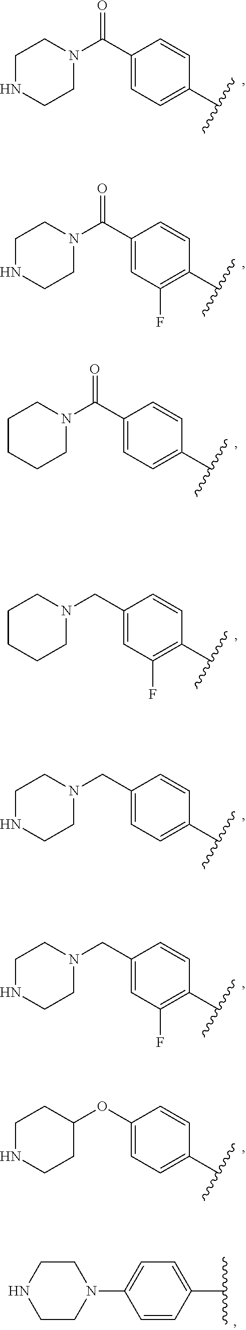

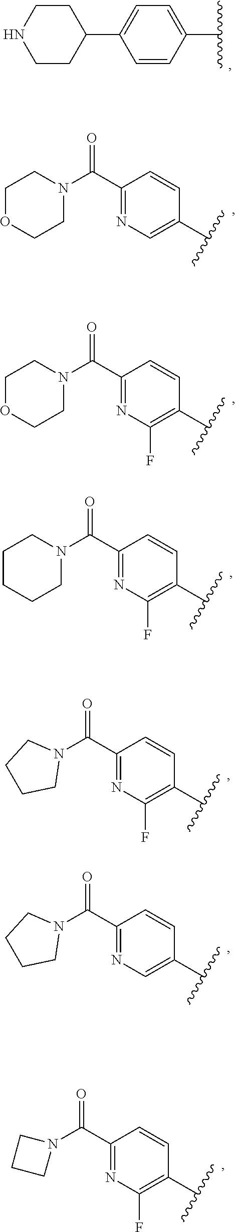

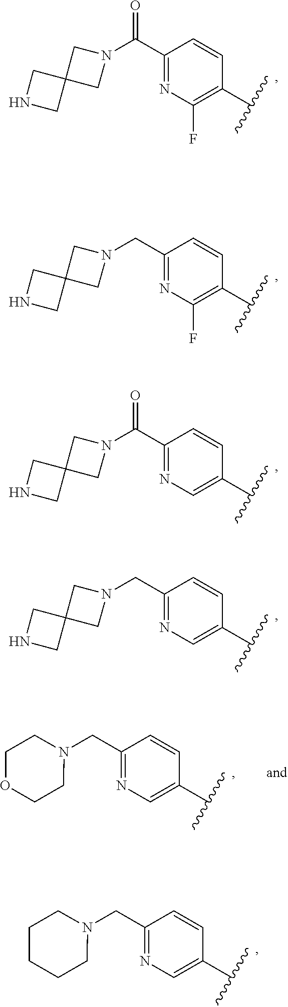

wherein each Z is optionally substituted with one or more R^{Za}

In some embodiments, n is 1, and Z-(p)ₘ-Ring B is selected from the group consisting of

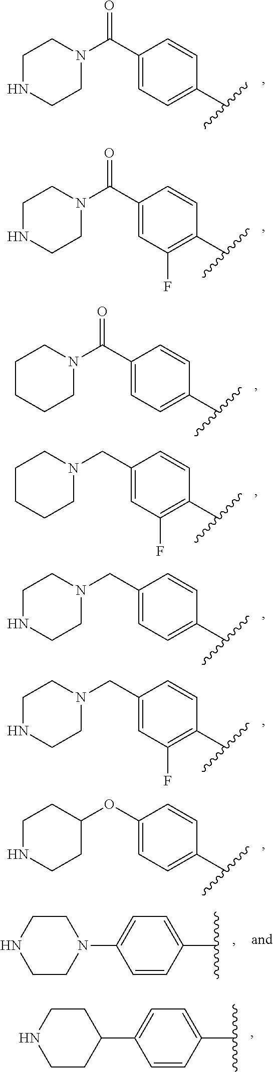

wherein each Z is optionally substituted with one or more R^{Za}. In some embodiments, Z-(p)ₘ-Ring B is

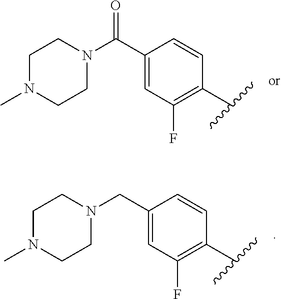 [SMILES: *c1ccc(C(=O)N2CCN(C)CC2)cc1F ; *c1ccc(CN2CCN(C)CC2)cc1F]

In some embodiments, Z-(p)ₘ-Ring B is

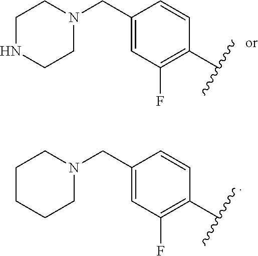 [SMILES: *c1ccc(CN2CCNCC2)cc1F ; *c1ccc(CN2CCCCC2)cc1F]

In some embodiments, Z-(p)ₘ-Ring B is

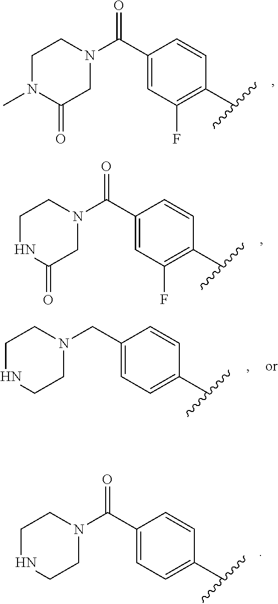

In some embodiments, n is 1, and Z-(p)ₘ-Ring B is

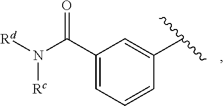

wherein each R^{c} and R^{d} is independently H or C₁₋₆ alkyl optionally substituted with one or more —OH, halo, —NH₂, —CF₃, or oxo. In some embodiments, n is 1, and Z-(p)ₘ-Ring B is

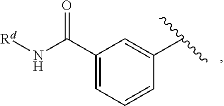

wherein R^{d} is C₁₋₆ alkyl optionally substituted with one or more —OH, halo, —NH₂, —CF₃, or oxo.

In some embodiments, Y is —NH— or —O—. In some embodiments, Y is —NH—. In some embodiments, Y is —O—.

In some embodiments, Ring A is selected from the group consisting of

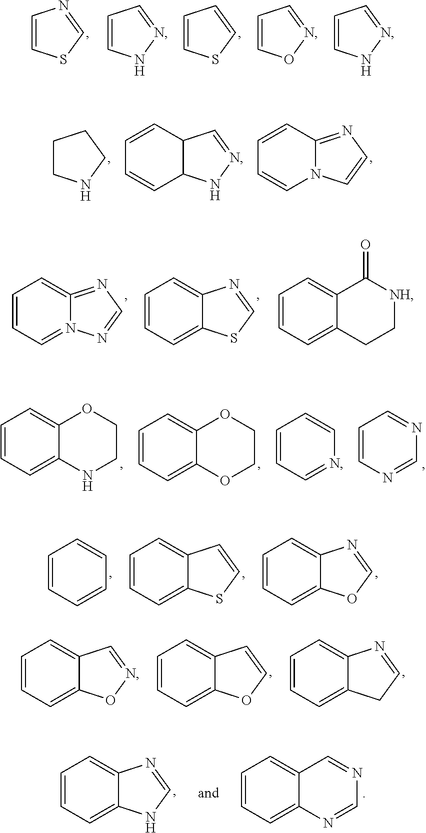

In some embodiments, Ring A is selected from the group consisting of

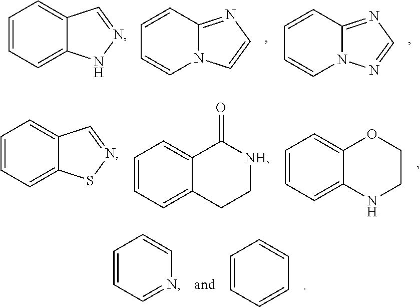

In some embodiments, Ring A is

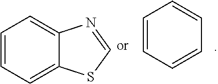

In some embodiments, Ring A is

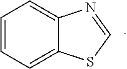

In some embodiments, Ring A is

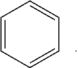

In some embodiments, Ring A is

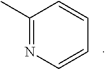

In some embodiments, Ring A is selected from the group consisting of

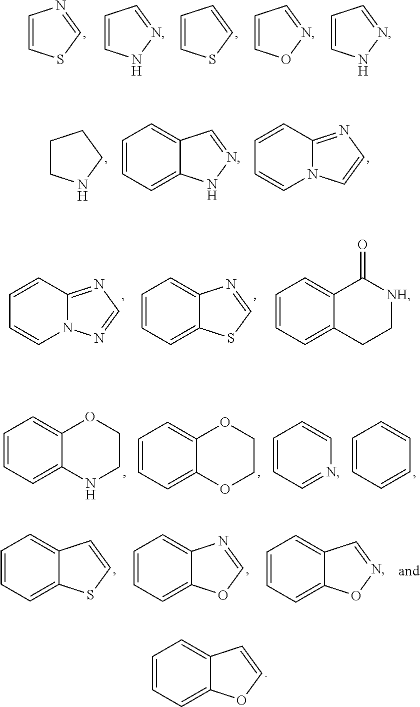

In some embodiments, Ring A is selected from the group consisting of

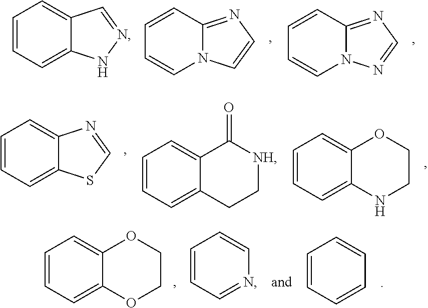

In some embodiments, Ring A is

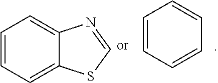

In some embodiments, Ring A is

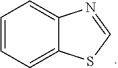

In some embodiments, Ring A is

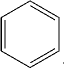

In some embodiments, Ring A is

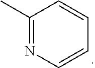

In some embodiments, Ring A s selected from the group consisting of

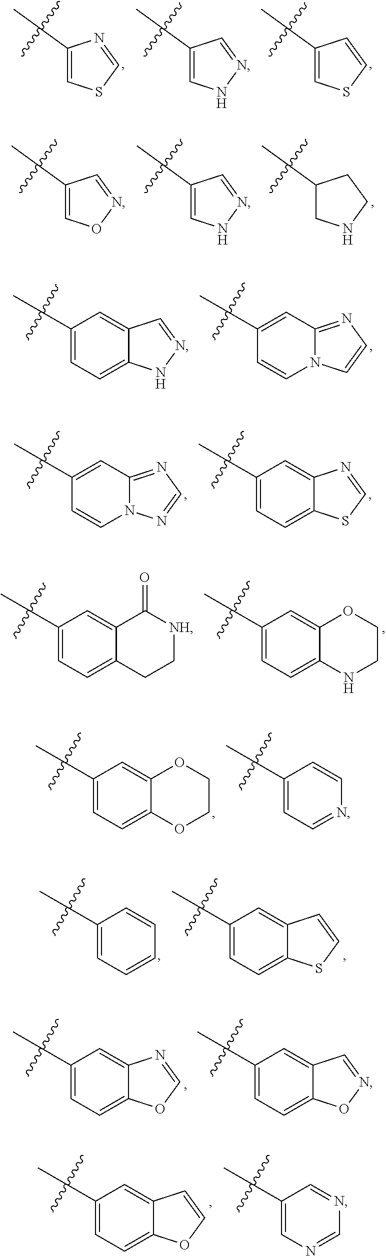

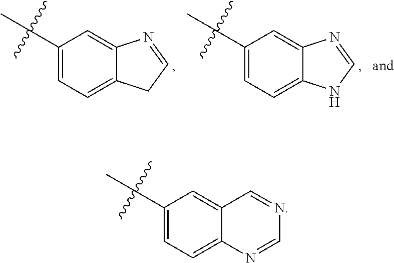 [SMILES: *c1ccc2c(c1)N=CC2 ; *c1ccc2[nH]cnc2c1 ; *c1ccc2ncncc2c1]

In some embodiments, Ring A is selected from the group consisting of

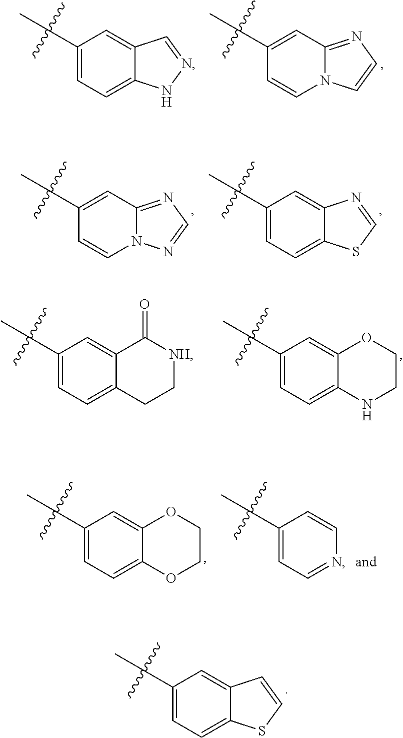

In some embodiments, Ring A is

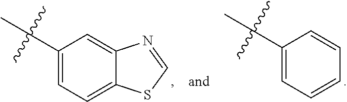 [SMILES: *c1ccc2scnc2c1 ; "," ; "and" ; *c1ccccc1 ; "."]

In some embodiments, Ring A is

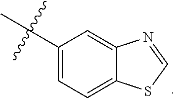 [SMILES: *c1ccc2scnc2c1]

In some embodiments, Ring A is

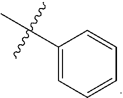 [SMILES: *c1ccccc1]

In some embodiments, Ring A is

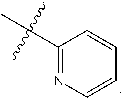 [SMILES: *c1ccccn1]

In some embodiments, Ring A is selected from the group consisting of

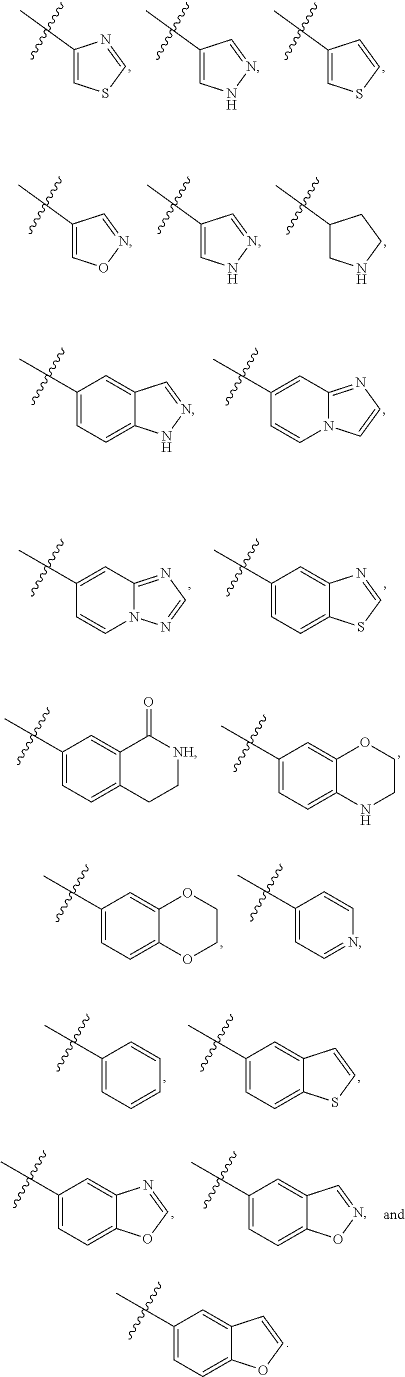

In some embodiments, Ring A is selected from the group consisting of

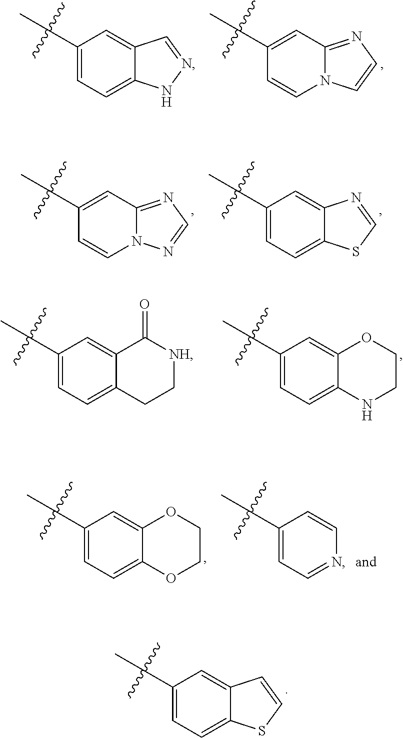

In some embodiments, Ring A is

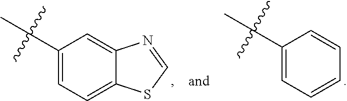 [SMILES: *c1ccc2scnc2c1 ; "," ; "and" ; *c1ccccc1 ; "."]

In some embodiments, Ring A is

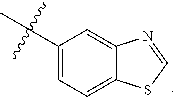 [SMILES: *c1ccc2scnc2c1]

In some embodiments, Ring A is

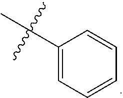 [SMILES: *c1ccccc1]

In some embodiments, Ring A is

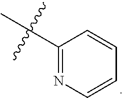 [SMILES: *c1ccccn1]

In some embodiments, n is 0 or 1, and Ring B—(R¹)_{q} is

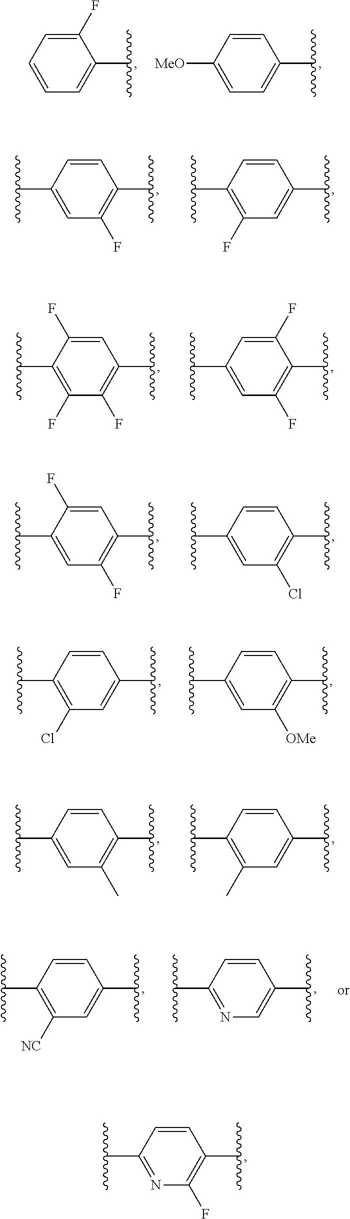

wherein each wavy line on the right indicates covalent attachment to Q, and each wavy light on the left indicates covalent attachment to Z-(p)ₘ-. In some embodiments, n is 0 or 1, and Ring B—(R¹)_{q} is

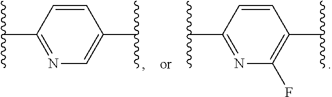 [SMILES: *c1ccc(*)nc1 ; *c1ccc(*)c(F)n1]

In some embodiments, n is 0 or 1, and Ring B—(R¹)_{q} is

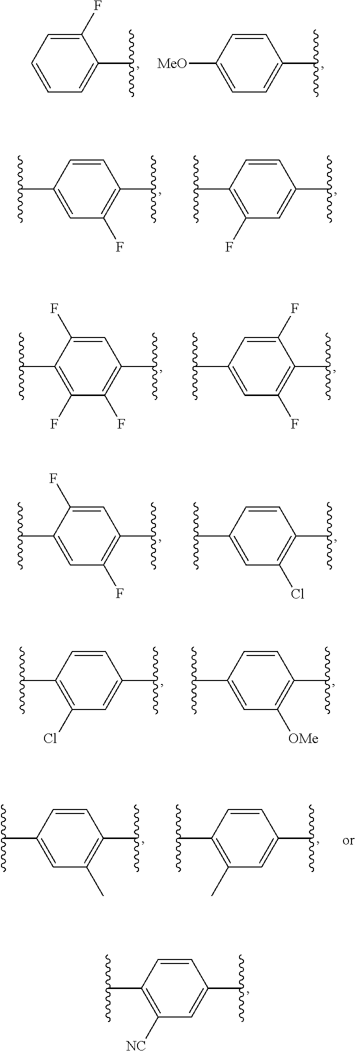

wherein each wavy line on the right indicates covalent attachment to Q, and each wavy light on the left indicates covalent attachment to Z-(p)ₘ-.

In some embodiments, each T is independently selected from the group consisting of oxo, halo, —NH₂, C₁-C₆ alkyl, C₁₋₆ alkoxy, —NHC(O)—C₃₋₆cycloalkyl, and methylpiperidyl. In some embodiments, each T is independently selected from the group consisting of halo, —NH₂, C₁-C₆ alkyl, C₁₋₆ alkoxy, —NHC(O)—C₃₋₆cycloalkyl, and methylpiperidyl. In some embodiments, each T is independently selected from the group consisting of halo, C₁-C₆ alkyl, and methylpiperidyl. In some embodiments, each T is halo. In some embodiments, each T is —NH₂. In some embodiments, each T is C₁-C₆ alkyl. In some embodiments, each T is C₁₋₆ alkoxy. In some embodiments, each T is —NHC(O)—C₃₋₆cycloalkyl. In some embodiments, each T is methylpiperidyl. In some embodiments, w is an integer from 1-5 and at least one T is halo. In some embodiments, at least one T is F. In some embodiments, at least one T is Cl. In some embodiments, w is an integer from 1-5 and at least one T is —NH₂. In some embodiments, w is an integer from 1-5 and at least one T is C₁-C₆ alkyl. In some embodiments, w is an integer from 1-5 and at least one T is NHC(O)—C₃₋₆cycloalkyl. In some embodiments, w is an integer from 1-5 and at least one T is methylpiperidyl. In some embodiments, w is an integer from 1-5 and at least one T is methoxy.

In some embodiments, each T is independently selected from the group consisting of halo, —NH₂, C₁-C₆ alkyl, C₁₋₆ alkoxy, —NHC(O)—C₃₋₆cycloalkyl, and methylpiperidyl. In some embodiments, each T is independently selected from the group consisting of halo, C₁-C₆alkyl, and methylpiperidyl. In some embodiments, each T is halo. In some embodiments, each T is —NH₂. In some embodiments, each T is C₁-C₆ alkyl. In some embodiments, each T is C₁₋₆ alkoxy. In some embodiments, each T is —NHC(O)—C₃₋₆cycloalkyl. In some embodiments, each T is methylpiperidyl. In some embodiments, w is an integer from 1-5 and at least one T is halo. In some embodiments, at least one T is F. In some embodiments, at least one T is Cl. In some embodiments, w is an integer from 1-5 and at least one T is —NH₂. In some embodiments, w is an integer from 1-5 and at least one T is C₁-C₆ alkyl. In some embodiments, w is an integer from 1-5 and at least one T is NHC(O)—C₃₋₆cycloalkyl. In some embodiments, w is an integer from 1-5 and at least one T is methylpiperidyl. In some embodiments, w is an integer from 1-5 and at least one T is methoxy.

In some embodiments, T is selected from the group consisting of —Cl, —F, —NH₂, —CH₃,

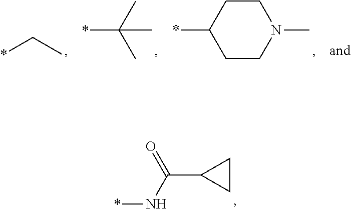

wherein, for each T, * denotes the point of attachment to Ring A.

In some embodiments, Ring A-[T]_{w} is selected from the group consisting of

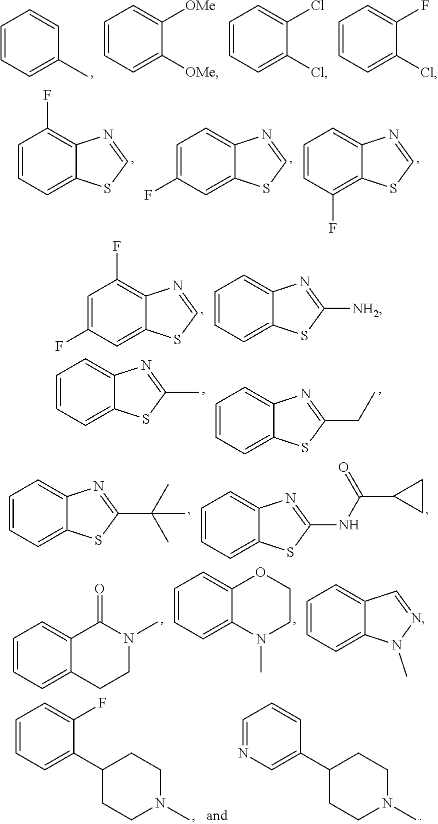

In some embodiments, Ring A-[T]_{w} is

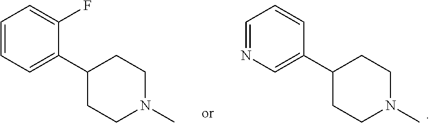

In some embodiments, Ring A-[T]_{w} is

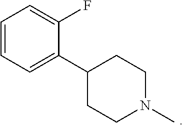

In some embodiments, Ring A-[T]_{w} is selected from the group consisting of

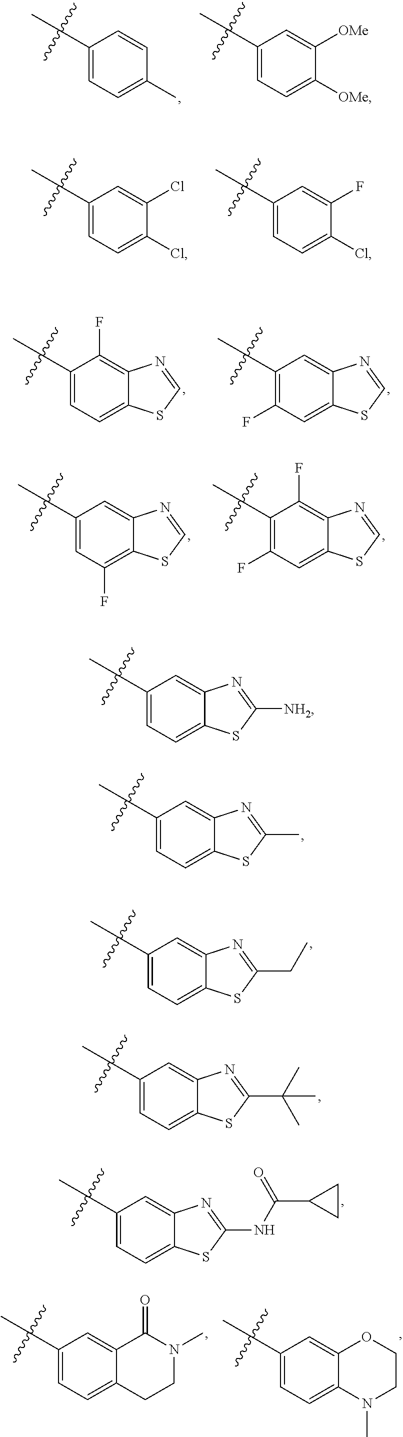

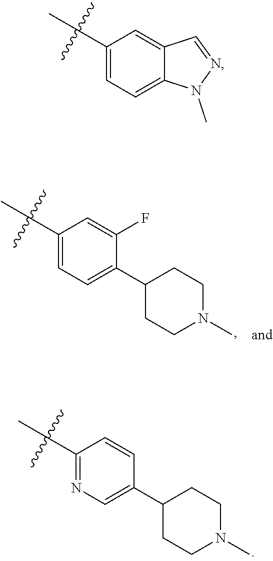 [SMILES: *c1ccc2c(cnn2C)c1 ; *c1ccc(C2CCN(C)CC2)c(F)c1 ; *c1ccc(C2CCN(C)CC2)cn1]

In some embodiments, Ring A-[T]_{w} is

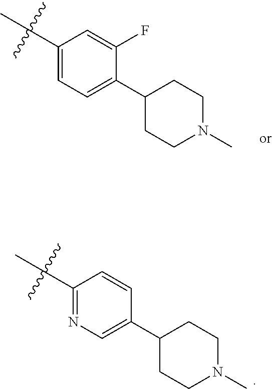 [SMILES: *c1ccc(C2CCN(C)CC2)c(F)c1 ; *c1ccc(C2CCN(C)CC2)cn1]

N or N In some embodiments, Ring A-[T]_{w} is

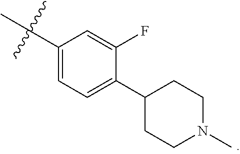 [SMILES: *c1ccc(C2CCN(C)CC2)c(F)c1]

In some embodiments, n+w is an integer greater than 0. In some embodiments, when Ring A is phenyl, then either: (a) n is an integer from 1 to 5 and Ring B is substituted by at least one (p)ₘ-Z wherein Z is 3- to 12-membered heterocyclyl substituted with one or more R^{Za} or (b) Ring A is substituted by at least one T wherein T is methylpiperidyl. In some embodiments, when Ring A is phenyl, then n is an integer from 1 to 5 and Ring B is substituted by at least one (p)ₘ-Z wherein Z is 3- to 12-membered heterocyclyl substituted with one or more R^{Za}. In some embodiments, when Ring A is phenyl, then n is 1 and Ring B is substituted by at least one (p)ₘ-Z wherein Z is 3- to 12-membered heterocyclyl substituted with one or more R^{Za}. In some embodiments, when Ring A is phenyl, then n is an integer from 1 to 5 and Ring B is substituted by at least one (p)ₘ-Z wherein Z is piperidinyl, piperazinyl, pyrrolidinyl, or morpholinyl, each of which is substituted with one or more R^{Za}In some embodiments, when Ring A is phenyl, then n is 1 Ring B is substituted by at least one (p)ₘ-Z wherein Z is piperidinyl or piperazinyl, each of which is substituted with one or more R^{Za}. In some embodiments, when Ring A is phenyl, then Ring A is substituted by at least one T wherein T is methylpiperidyl. In some embodiments, when Ring B is phenyl, m is 0, and Z is piperazinyl, at least one R^{Za} is present and is CH₃ or oxo. In some embodiments, when Ring B is phenyl, m is 0, and Z is piperazinyl, Z is substituted with CH₃. In some embodiments, when Ring B is phenyl, m is 0, and Z is piperazinyl, Z is substituted with oxo. In some embodiments, when Ring B is phenyl, m is 0, and Z is piperazinyl, Z is substituted with CH₃ and oxo. In some embodiments, B is phenyl, and Z is piperazinyl substituted with one or more substituents selected from the group consisting of CH₃ and oxo. In some embodiments, B is phenyl substituted with halo, and Z is piperazinyl substituted with one or more substituents selected from the group consisting of CH₃ and oxo. In some embodiments, B is phenyl substituted with fluoro, and Z is piperazinyl substituted with one or more substituents selected from the group consisting of CH₃ and oxo. In some embodiments, m is 0, B is phenyl, and Z is piperazinyl substituted with one or more substituents selected from the group consisting of CH₃ and oxo. In some embodiments, m is 0, B is phenyl substituted with halo, and Z is piperazinyl substituted with one or more substituents selected from the group consisting of CH₃ and oxo. In some embodiments, m is 0, B is phenyl substituted with fluoro, and Z is piperazinyl substituted with one or more substituents selected from the group consisting of CH₃ and oxo.

In some embodiments, n+w is an integer greater than 0. In some embodiments, when Ring A is phenyl, then either: (a) n is an integer from 1 to 5 and Ring B is substituted by at least one (p)ₘ-Z wherein Z is 3- to 12-membered heterocyclyl substituted with one or more R^{Za} or (b) Ring A is substituted by at least one T wherein T is methylpiperidyl. In some embodiments, when Ring A is phenyl, then n is an integer from 1 to 5 and Ring B is substituted by at least one (p)ₘ-Z wherein Z is 3- to 12-membered heterocyclyl substituted with one or more R^{Za}. In some embodiments, when Ring A is phenyl, then n is 1 and Ring B is substituted by at least one (p)ₘ-Z wherein Z is 3- to 12-membered heterocyclyl substituted with one or more R^{Za}. In some embodiments, when Ring A is phenyl, then n is an integer from 1 to 5 and Ring B is substituted by at least one (p)ₘ-Z wherein Z is piperidinyl, piperazinyl, pyrrolidinyl, or morpholinyl, each of which is substituted with one or more R^{Za}In some embodiments, when Ring A is phenyl, then n is 1 Ring B is substituted by at least one (p)ₘ-Z wherein Z is piperidinyl or piperazinyl, each of which is substituted with one or more R^{Za}. In some embodiments, when Ring A is phenyl, then Ring A is substituted by at least one T wherein T is methylpiperidyl.

In one aspect, provided is a compound of Formula (I), or a pharmaceutically acceptable salt, stereoisomer, or tautomer thereof, wherein the compound or the pharmaceutically acceptable salt, stereoisomer, or tautomer thereof has any one or more of the following structural features, provided that features (iv) and (v) cannot be combined, features (vi) and (vii) cannot be combined, and features (xi) and (xii) cannot be combined:
(i) Q is a bond;
(ii) Ring B is phenyl;
(iii) q is 1 and R¹ is fluoro;
(iv) m is 1 and p is methylene;
(v) m is 1 and p is —C(O)—;
(vi) n is 1 and Z is piperidinyl;
(vii) n is 1 and Z is piperazinyl;
(viii) at least one R^{Za}, R^{Zb}, or R^{Zc} is methyl;
(ix) at least one R^{Za}, R^{Zb}, or R^{Zc} is oxo;
(x) Y is N;
(xi) Ring A is phenyl; and
(xii) Ring A is benzothiazolyl.

It is understood that compounds of formula (I) or any variation thereof described herein, or a pharmaceutically acceptable salt, stereoisomer, or tautomer thereof, can in one embodiment have any one or more of the structural features as noted above. For example, compounds of formula (I) or any variation thereof described herein, or a pharmaceutically acceptable salt, stereoisomer, or tautomer thereof, can in one embodiment have the following structural features: (i) and any one or two or eight of (ii), (iii), (iv), (v), (vi), (vii), (viii), (ix), (x), (xi), and (xii), provided that features (iv) and (v) cannot be combined, features (vi) and (vii) cannot be combined, and features (xi) and (xii) cannot be combined. In one such example, a compound of formula (I) or any variation thereof described herein, or a pharmaceutically acceptable salt, stereoisomer, or tautomer thereof, can in one embodiment have the following structural features: (i), (v), (vii), (xii), and any one or two or all of (ii), (iii), (viii), (ix), and (x). Although specific combinations of structural features are specifically noted below, it is understood that each and every combination of features is embraced, provided that features (iv) and (v) cannot be combined, features (vi) and (vii) cannot be combined, and features (xi) and (xii) cannot be combined. In one variation, features (i), (ii), (iii), (iv), (vii), (x), and (xii) apply. In another variation, features (i), (ii), (v), (vii), (viii), (ix), (x), and (xii) apply. In another variation, features (i), (x), and (xi) apply. In another variation, features (ii), (iii), (vi), (viii), (x), and (xii) apply. In another variation, features (i), (ii), (iii), (iv), (vii), (ix), and (xii) apply.

In another aspect, provided is a compound of Formula (Ia), or a pharmaceutically acceptable salt, stereoisomer, or tautomer thereof, wherein the compound or the pharmaceutically acceptable salt, stereoisomer, or tautomer thereof has any one or more of the following structural features, provided that features (iv) and (v) cannot be combined, features (vi) and (vii) cannot be combined, and features (xi) and (xii) cannot be combined: (i) Q is a bond;
(ii) Ring B is pyridyl;
(iii) q is 0;
(iv) m is 1 and p is methylene;
(v) m is 1 and p is —C(O)—;
(vi) n is 1 and Z is piperidinyl;
(vii) n is 1 and Z is morpholinyl;
(viii) at least one R^{Za}, R^{Zb}, or R^{Zc} is methyl;
(ix) at least one R^{Za}, R^{Zb}, or R^{Zc} is oxo;
(x) Y is N;
(xi) Ring A is phenyl; and
(xii) Ring A is benzothiazolyl.

It is understood that compounds of formula (Ia) or any variation thereof described herein, or a pharmaceutically acceptable salt, stereoisomer, or tautomer thereof, can in one embodiment have any one or more of the structural features as noted above. For example, compounds of formula (Ia) or any variation thereof described herein, or a pharmaceutically acceptable salt, stereoisomer, or tautomer thereof, can in one embodiment have the following structural features: (i) and any one or two or eight of (ii), (iii), (iv), (v), (vi), (vii), (viii), (ix), (x), (xi), and (xii), provided that features (iv) and (v) cannot be combined, features (vi) and (vii) cannot be combined, and features (xi) and (xii) cannot be combined. In one such example, a compound of formula (Ia) or any variation thereof described herein, or a pharmaceutically acceptable salt, stereoisomer, or tautomer thereof, can in one embodiment have the following structural features: (i), (v), (vii), (xii), and any one or two or all of (ii), (iii), (viii), (ix), and (x). Although specific combinations of structural features are specifically noted below, it is understood that each and every combination of features is embraced, provided that features (iv) and (v) cannot be combined, features (vi) and (vii) cannot be combined, and features (xi) and (xii) cannot be combined. In one variation, features (i), (ii), (iii), (v), (vii), (x), and (xii) apply. In another variation, features (i), (ii), (v), (vi), (viii), (x), and (xii) apply. In another variation, features (i), (x), and (xi) apply. In another variation, features (ii), (iii), (vi), (viii), (x), and (xii) apply. In another variation, features (i), (ii), (iii), (iv), (vii), (ix), and (xii) apply.

In some embodiments, the compound of Formula (Ia) is a compound of Formula (II-1):

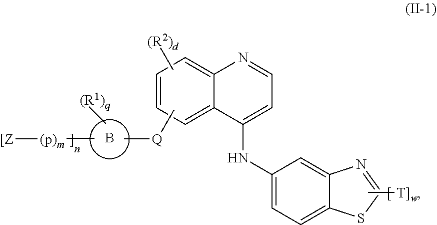

or a pharmaceutically acceptable salt, stereoisomer, or tautomer thereof, wherein d, m, n, q, w, Q, Ring B, R¹, R², p, Z, and T are each defined as herein for Formula (Ia) or Formula (I).

In some embodiments, the compound of Formula (Ia) is a compound of Formula (II-2):

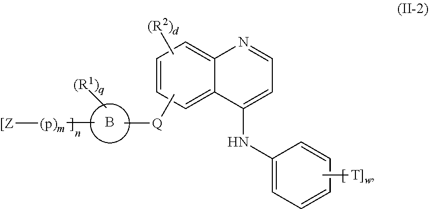

or a pharmaceutically acceptable salt, stereoisomer, or tautomer thereof, wherein d, m, n, q, w, Q, Ring B, R¹, R², p, Z, and T are each defined as herein for Formula (Ia) or Formula (I).

In some embodiments, the compound of Formula (Ia) is a compound of Formula (III-1):

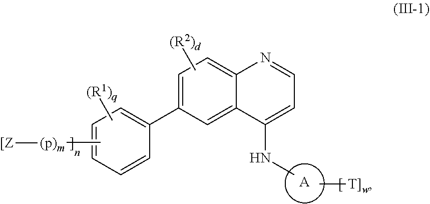

or a pharmaceutically acceptable salt, stereoisomer, or tautomer thereof, wherein d, m, n, q, w, R¹, R², p, Z, Ring A, and T are each defined as herein for Formula (Ia) or Formula (I).

In some embodiments, the compound of Formula (Ia) is a compound of Formula (III-2):

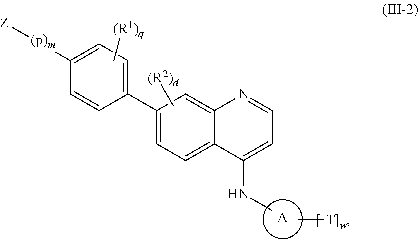

or a pharmaceutically acceptable salt, stereoisomer, or tautomer thereof, wherein d, m, q, w, R¹, R², p, Z, Ring A, and T are each defined as herein for Formula (Ia) or Formula (I).

In some embodiments of a compound of Formula (III-1) or (III-2), q is 1, and R¹ and Z-(p)ₘ are positioned in a 1,3- or 1,4-relationship to each other (i.e. meta or para). In some embodiments, q is 1, and R¹ and Z-(p)ₘ are positioned in a 1,3-relationship to each other (i.e. meta).

In some embodiments of a compound of Formula (III-1) or (III-2), d is 0; m is 0, 1, or 2, wherein when m is 1 or 2, each p is independently selected from the group consisting of C₁₋₆ alkylene, —O—, and —C(O)—; Z is piperazinyl, piperidinyl, pyrrolidinyl, morpholinyl.

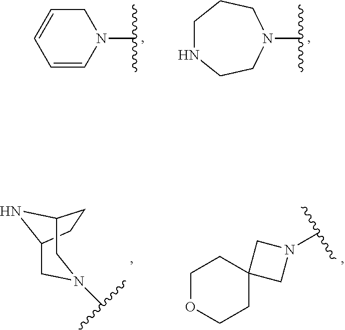 [SMILES: *N1C=CC=CC1 ; *N1CCCNCC1 ; *N1CC2CCC(C1)N2 ; *N1CC2(CCOCC2)C1]

or —N(CH₃)₂; each R^{Za} is independently selected from the group consisting of C₁₋₆ alkyl, oxo, —CF₃, halo, —C(O)OR^{c}, C(O)R^{c}, and

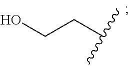 [SMILES: *CCO]

q is 0 or 1, wherein when q is 1, R¹ is halo, C₁₋₆ alkoxy, or cyano; Ring A is

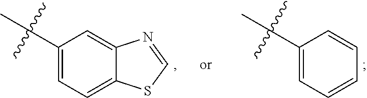 [SMILES: *c1ccc2scnc2c1 ; *c1ccccc1]

and w is 0 or 1, wherein when w is 1, T is selected from the group consisting of methyl, —NH₂, fluoro, and chloro.

In some embodiments of a compound of Formula (III-1) or (III-2), d is 0; m is 0, 1, or 2, wherein when m is 1 or 2, each p is independently selected from the group consisting of methylene, —O—, and —C(O)—; Z is

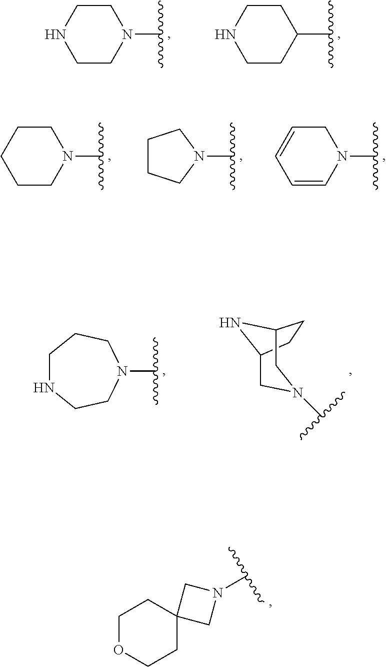

or N(CH₃)₂; each R^{Za} is independently selected from the group consisting of methyl, oxo, —CF₃, fluoro,

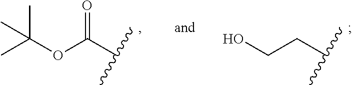

q is 0 or 1, wherein when q is 1, R¹ is fluoro, methoxy, or cyano; Ring A is

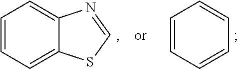 [SMILES: c1ccc2scnc2c1 ; c1ccccc1]

and w is 0 or 1, wherein when w is 1, T is selected from the group consisting of methyl, NH₂, fluoro, and chloro.

In some embodiments of a compound of Formula (III-1) or (III-2), d is 0; q is 0 or 1, wherein when q is 1, R¹ is fluoro or methoxy; w is 0, 1, or 2, wherein when w is 1 or 2, each T is independently fluoro or chloro; m is 1 or 2; each p is independently methylene, —C(O)—, or —O—; Z is piperazinyl, piperidinyl, pyrrolidinyl,

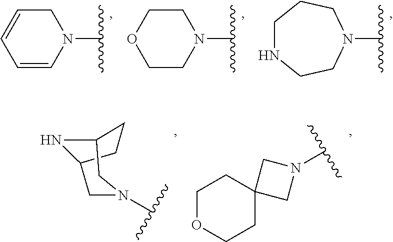

or N(CH₃)₂; each R^{Za} is independently selected from the group consisting of methyl, oxo,

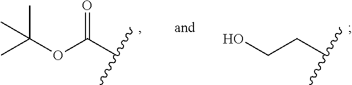

### and Ring A is

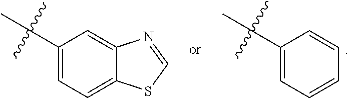 [SMILES: *c1ccc2scnc2c1 ; *c1ccccc1]

In some embodiments, the compound of Formula (Ia) is a compound of Formula (IV-1):

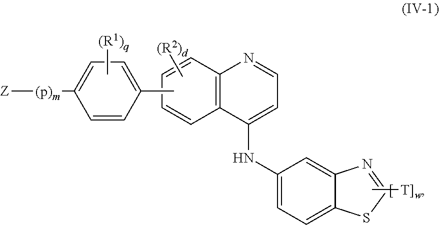

or a pharmaceutically acceptable salt, stereoisomer, or tautomer thereof, wherein d, m, q, w, R¹, R², p, Z, and T are each defined as herein for Formula (Ia) or (I).

In some embodiments of a compound of Formula (Ia), (I), (II-1), or (IV-1), Z is piperazinyl optionally substituted with one or more substituents selected from the group consisting of oxo and methyl. In some embodiments, m is 1, and p is alkylene. In some embodiments, m is 1, and p is methylene. In some embodiments, q is 1, and R¹ is fluoro. In some embodiments, d is 0. In some embodiments, Z is piperazinyl, m is 1, and p is methylene. In some embodiments, m is 1, p is methylene, and d is 0. In some embodiments, Z is piperidinyl. In some embodiments, w is 1, and T is fluoro. In some embodiments, Z is piperazinyl optionally substituted with one or more substituents selected from the group consisting of oxo and methyl, m is 1, p is methylene, d is 0, q is 1, and w is 0.

In some embodiments of a compound of Formula (Ia), (I), (II-1), or (IV-1), d is 0; q is 0 or 1, wherein when q is 1, R¹ is fluoro; w is 0; m is 1 or 2; each p is independently methylene, —C(O)—, or —O—; Z is piperazinyl, piperidinyl, pyrrolidinyl,

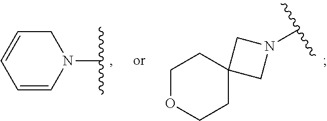 [SMILES: *N1C=CC=CC1 ; *N1CC2(CCOCC2)C1]

and each R^{Za} is independently selected from the group consisting of methyl, oxo, and

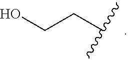 [SMILES: *CCO]

In some embodiments of a compound of Formula (Ia), (I), (II-1), or (IV-1), Z-(p)ₘ-Ring B is selected from the group consisting of

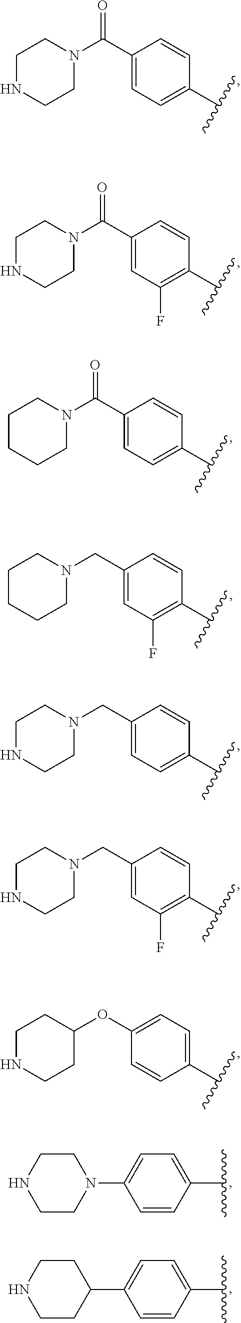

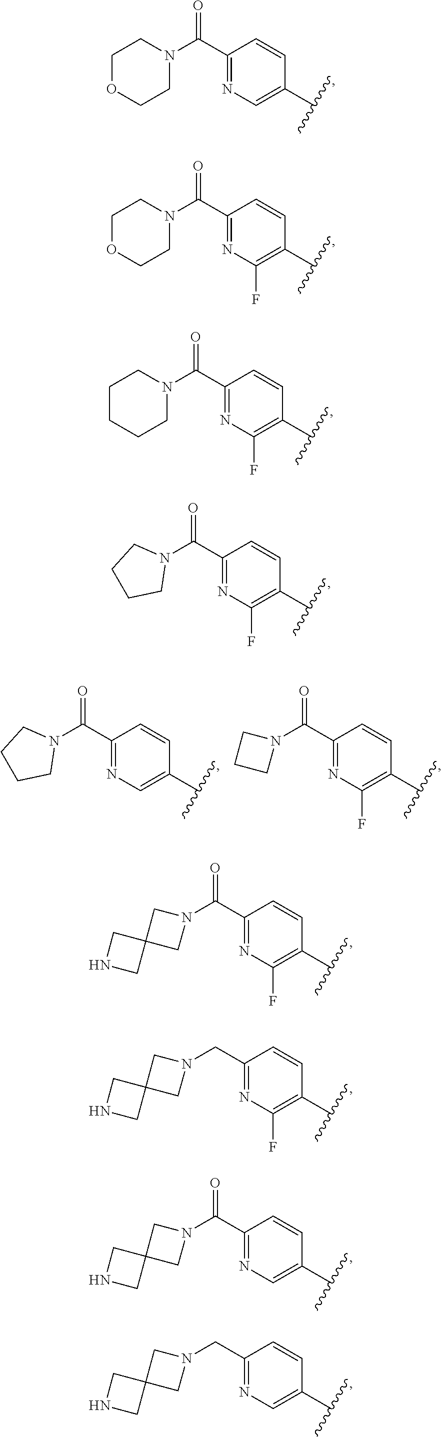

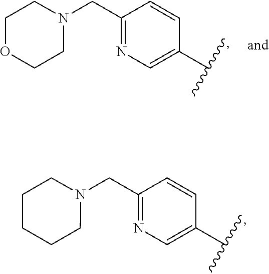

wherein each Z is optionally substituted with one or more R^{Za}.

In some embodiments of a compound of Formula (Ia), (I), (II-1), or (IV-1), Z-(p)ₘ-Ring B is selected from the group consisting of

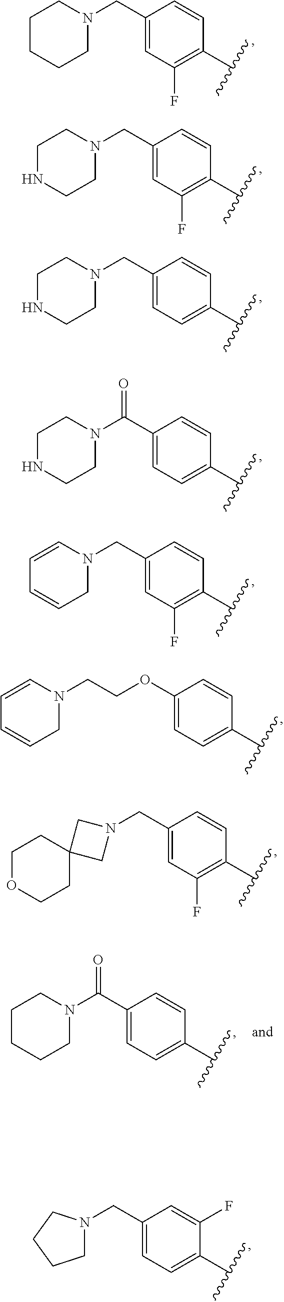

wherein each R^{Za} is independently selected from the group consisting of
methyl, oxo, and

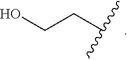 [SMILES: *CCO]

In some embodiments of a compound of Formula (Ia), (I), (II-1), or (IV-1), Z-(p)ₘ-Ring B is selected from the group consisting of

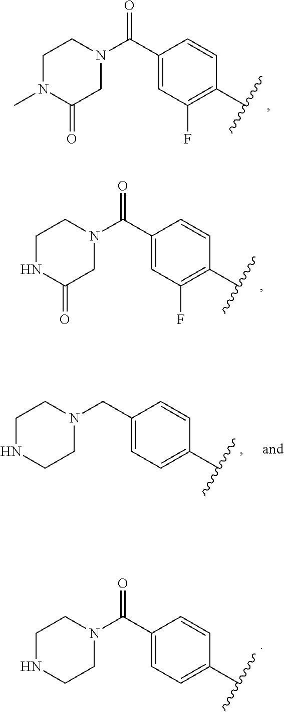 [SMILES: *c1ccc(C(=O)N2CCN(C)C(=O)C2)cc1F ; *c1ccc(C(=O)N2CCNC(=O)C2)cc1F ; *c1ccc(CN2CCNCC2)cc1 ; *c1ccc(C(=O)N2CCNCC2)cc1]

In some embodiments, the compound of Formula (Ia) is a compound of Formula (IV-2):

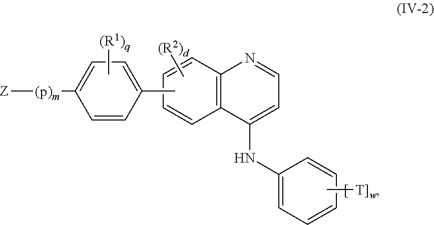

or a pharmaceutically acceptable salt, stereoisomer, or tautomer thereof, wherein d, m, q, w, R¹, R², p, Z, and T are each defined as herein for Formula (Ia) or Formula (I).

In some embodiments of a compound of Formula (IV-1) or (IV-2), q is 1, and R¹ and Z-(p)ₘ are positioned in a 1,3- or 1,4-relationship to each other (i.e. meta or para). In some embodiments, q is 1, and R¹ and Z-(p)ₘ are positioned in a 1,3-relationship to each other (i.e. meta).

In some embodiments, provided herein are compounds, or a pharmaceutically acceptable salt, stereoisomer, or tautomer thereof, described in Table 1.

TABLE 1

| | Ex- | | |
|---|---|---|---|
| Cpd. | ample | | |
| No. | No. | Structure | Compound Name |
| 1 | 1 | 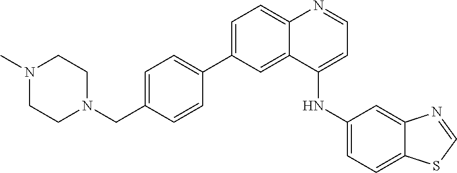 [SMILES: CN1CCN(Cc2ccc(-c3ccc4nccc(Nc5ccc6scnc6c5)c4c3)cc2)CC1] | N-(6-(4-((4- methylpiperazin-1- yl)methyl)phenyl)quinolin- 4-yl)benzo[d]thiazol-5- amine |
| 2 | 2 | 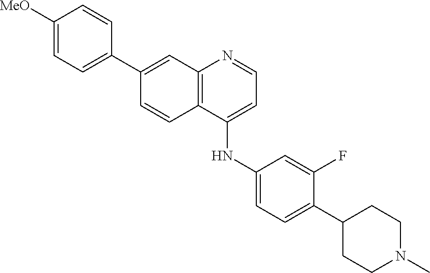 [SMILES: COc1ccc(-c2ccc3c(Nc4ccc(C5CCN(C)CC5)c(F)c4)ccnc3c2)cc1] | N-(3-fluoro-4-(1- methylpiperidin-4- yl)phenyl)-7-(4- methoxyphenyl)quinolin- 4-amine |
| 3 | 3 | 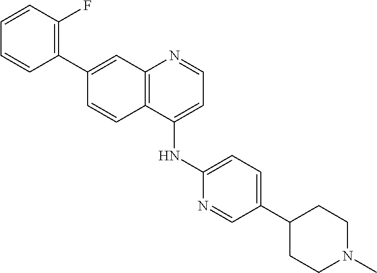 [SMILES: CN1CCC(c2ccc(Nc3ccnc4cc(-c5ccccc5F)ccc34)nc2)CC1] | 7-(2-fluorophenyl)-N-(5- (1-methylpiperidin-4- yl)pyridin-2-yl)quinolin-4- amine |
| 4 | 4 | 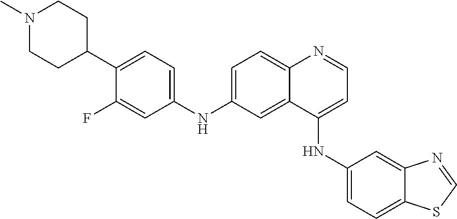 [SMILES: CN1CCC(c2ccc(Nc3ccc4nccc(Nc5ccc6scnc6c5)c4c3)cc2F)CC1] | 4-N-(1,3-benzothiazol-5- yl)-6-N-[3-fluoro-4-(1- methylpiperidin-4- yl)phenyl]quinoline-4,6- diamine |
| 5 | 5 | 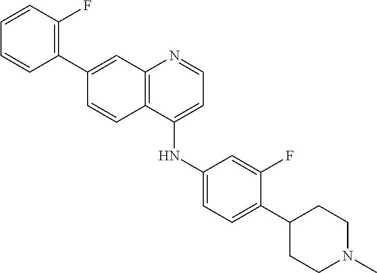 [SMILES: CN1CCC(c2ccc(Nc3ccnc4cc(-c5ccccc5F)ccc34)cc2F)CC1] | N-(3-fluoro-4-(1- methylpiperidin-4- yl)phenyl)-7-(2- fluorophenyl)quinolin-4- amine |
| 6 | 6 | 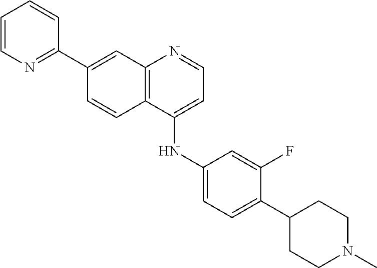 [SMILES: CN1CCC(c2ccc(Nc3ccnc4cc(-c5ccccn5)ccc34)cc2F)CC1] | N-(3-fluoro-4-(1- methylpiperidin-4- yl)phenyl)-7-(pyridin-2- yl)quinolin-4-amine |
| 7 | 7 | 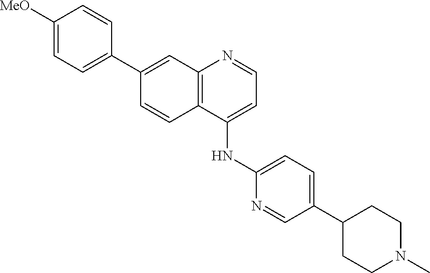 [SMILES: COc1ccc(-c2ccc3c(Nc4ccc(C5CCN(C)CC5)cn4)ccnc3c2)cc1] | 7-(4-methoxyphenyl)-N- (5-(1-methylpiperidin-4- yl)pyridin-2-yl)quinolin-4- amine |
| 8 | 8 | 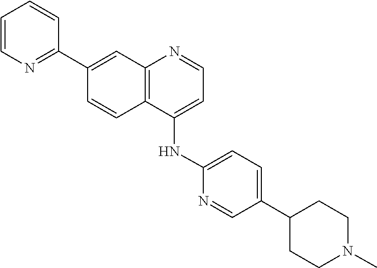 [SMILES: CN1CCC(c2ccc(Nc3ccnc4cc(-c5ccccn5)ccc34)nc2)CC1] | N-(5-(1-methylpiperidin- 4-yl)pyridin-2-yl)-7- (pyridin-2-yl)quinolin-4- amine |
| 9 | 9 | 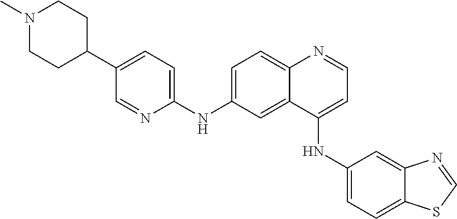 [SMILES: CN1CCC(c2ccc(Nc3ccc4nccc(Nc5ccc6scnc6c5)c4c3)nc2)CC1] | 4-N-(1,3-benzothiazol-5- yl)-6-N-[5-(1- methylpiperidin-4- yl)pyridin-2-yl]quinoline- 4,6-diamine |
| 10 | 10 | 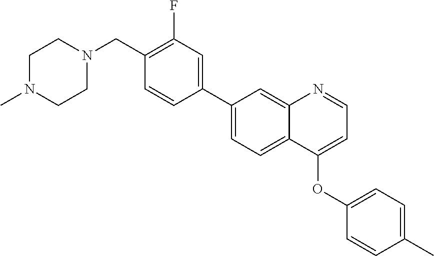 | 7-(3-fluoro-4-((4- methylpiperazin-1- yl)methyl)phenyl)-4-(p- tolyloxy)quinoline |
| 11 | 11 | 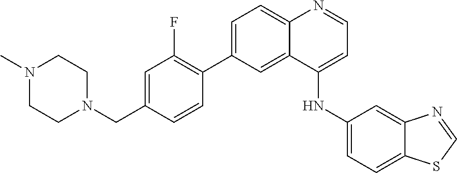 [SMILES: CN1CCN(Cc2ccc(-c3ccc4nccc(Nc5ccc6scnc6c5)c4c3)c(F)c2)CC1] | N-(6-(2-fluoro-4-((4- methylpiperazin-1- yl)methyl)phenyl)quinolin- 4-yl)benzo[d]thiazol-5- amine |
| 12 | 12 | 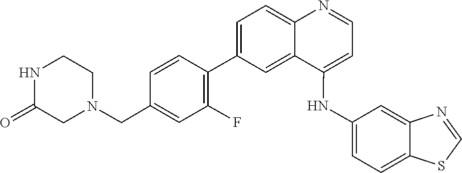 [SMILES: O=C1CN(Cc2ccc(-c3ccc4nccc(Nc5ccc6scnc6c5)c4c3)c(F)c2)CCN1] | 4-(4-(4-(benzo[d]thiazol- 5-ylamino)quinolin-6-yl)- 3-fluorobenzyl)piperazin- 2-one |
| 13 | 13 | 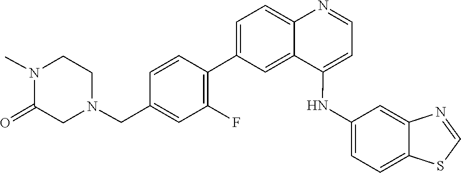 [SMILES: CN1CCN(Cc2ccc(-c3ccc4nccc(Nc5ccc6scnc6c5)c4c3)c(F)c2)CC1=O] | 4-(4-(4-(benzo[d]thiazol- 5-ylamino)quinolin-6-yl)- 3-fluorobenzyl)-1- methylpiperazin-2-one |
| 14 | 14 | 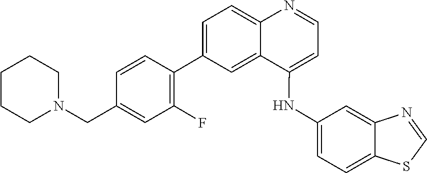 [SMILES: Fc1cc(CN2CCCCC2)ccc1-c1ccc2nccc(Nc3ccc4scnc4c3)c2c1] | N-(6-(2-fluoro-4- (piperidin-1- ylmethyl)phenyl)quinolin- 4-yl)benzo[d]thiazol-5- amine |
| 15 | 15 | 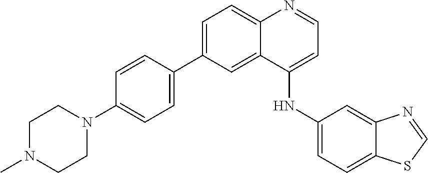 [SMILES: CN1CCN(c2ccc(-c3ccc4nccc(Nc5ccc6scnc6c5)c4c3)cc2)CC1] | N-(6-(4-(4- methylpiperazin-1- yl)phenyl)quinolin-4- yl)benzo[d]thiazol-5- amine |
| 16 | 16 | 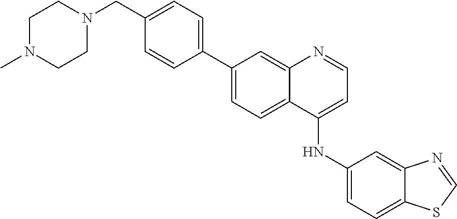 [SMILES: CN1CCN(Cc2ccc(-c3ccc4c(Nc5ccc6scnc6c5)ccnc4c3)cc2)CC1] | N-(7-(4-((4- methylpiperazin-1- yl)methyl)phenyl)quinolin- 4-yl)benzo[d]thiazol-5- amine |
| 17 | 17 | 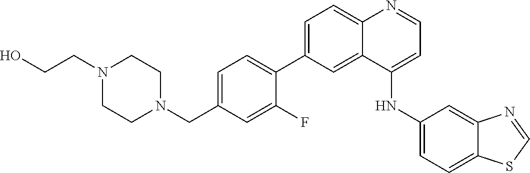 [SMILES: OCCN1CCN(Cc2ccc(-c3ccc4nccc(Nc5ccc6scnc6c5)c4c3)c(F)c2)CC1] | 2-(4-(4-(4- (benzo[d]thiazol-5- ylamino)quinolin-6-yl)-3- fluorobenzyl)piperazin-1- yl)ethan-1-ol |
| 18 | 18 | 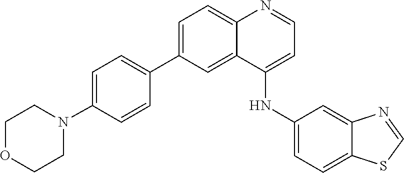 [SMILES: c1cc(Nc2ccc3scnc3c2)c2cc(-c3ccc(N4CCOCC4)cc3)ccc2n1] | N-(6-(4- morpholinophenyl)quinolin- 4-yl)benzo[d]thiazol-5- amine |
| 19 | 19 | 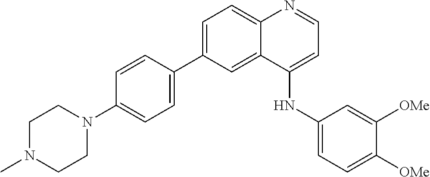 [SMILES: COc1ccc(Nc2ccnc3ccc(-c4ccc(N5CCN(C)CC5)cc4)cc23)cc1OC] | N-(3,4-dimethoxyphenyl)- 6-(4-(4-methylpiperazin-1- yl)phenyl)quinolin-4- amine |
| 20 | 20 | 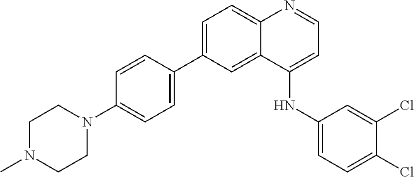 [SMILES: CN1CCN(c2ccc(-c3ccc4nccc(Nc5ccc(Cl)c(Cl)c5)c4c3)cc2)CC1] | N-(3,4-dichlorophenyl)-6- (4-(4-methylpiperazin-1- yl)phenyl)quinolin-4- amine |
| 21 | 21 | 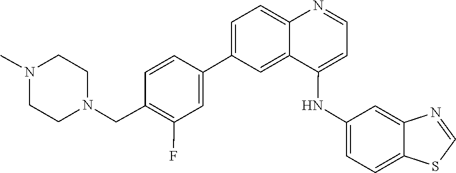 [SMILES: CN1CCN(Cc2ccc(-c3ccc4nccc(Nc5ccc6scnc6c5)c4c3)cc2F)CC1] | N-(6-(3-fluoro-4-((4- methylpiperazin-1- yl)methyl)phenyl)quinolin- 4-yl)benzo[d]thiazol-5- amine |
| 22 | 22 | 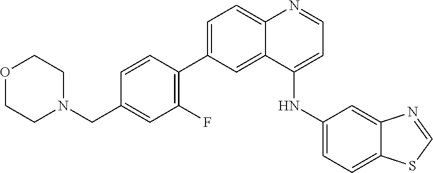 [SMILES: Fc1cc(CN2CCOCC2)ccc1-c1ccc2nccc(Nc3ccc4scnc4c3)c2c1] | N-(6-(2-fluoro-4- (morpholinomethyl)phenyl) quinolin-4- yl)benzo[d]thiazol-5- amine |
| 23 | 23 | 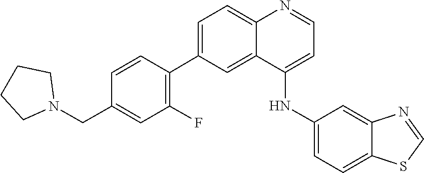 [SMILES: Fc1cc(CN2CCCC2)ccc1-c1ccc2nccc(Nc3ccc4scnc4c3)c2c1] | N-(6-(2-fluoro-4- (pyrrolidin-1- ylmethyl)phenyl)quinolin- 4-yl)benzo[d]thiazol-5- amine |
| 24 | 24 | 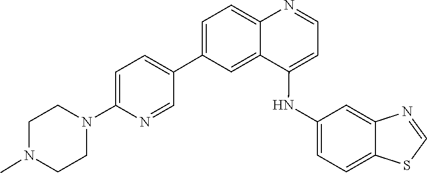 [SMILES: CN1CCN(c2ccc(-c3ccc4nccc(Nc5ccc6scnc6c5)c4c3)cn2)CC1] | N-(6-(6-(4- methylpiperazin-1- yl)pyridin-3-yl)quinolin-4- yl)benzo[d]thiazol-5- amine |
| 25 | 25 | 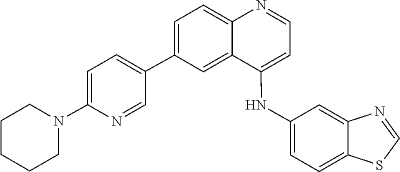 [SMILES: c1cc(Nc2ccc3scnc3c2)c2cc(-c3ccc(N4CCCCC4)nc3)ccc2n1] | N-(6-(6-(piperidin-1- yl)pyridin-3-yl)quinolin-4- yl)benzo[d]thiazol-5- amine |
| 26 | 26 | 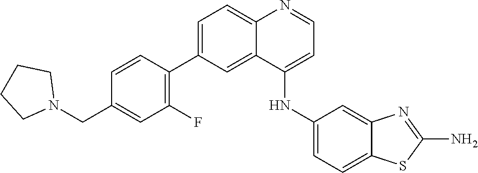 [SMILES: Nc1nc2cc(Nc3ccnc4ccc(-c5ccc(CN6CCCC6)cc5F)cc34)ccc2s1] | 5-N-[6-[2-fluoro-4- (pyrrolidin-1- ylmethyl)phenyl]quinolin- 4-yl]-1,3-benzothiazole- 2,5-diamine |
| 27 | 27 | 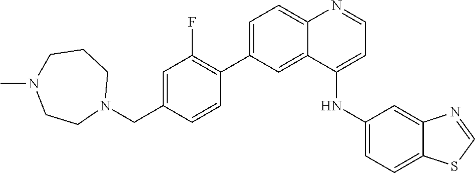 [SMILES: CN1CCCN(Cc2ccc(-c3ccc4nccc(Nc5ccc6scnc6c5)c4c3)c(F)c2)CC1] | N-(6-(2-fluoro-4-((4- methyl-1,4-diazepan-1- yl)methyl)phenyl)quinolin- 4-yl)benzo[d]thiazol-5- amine |
| 28 | 28 | 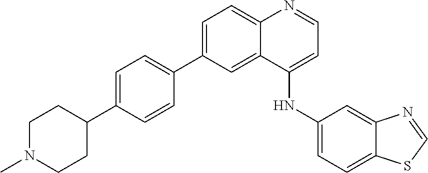 [SMILES: CN1CCC(c2ccc(-c3ccc4nccc(Nc5ccc6scnc6c5)c4c3)cc2)CC1] | N-(6-(4-(1- methylpiperidin-4- yl)phenyl)quinolin-4- yl)benzo[d]thiazol-5- amine |
| 29 | 29 | 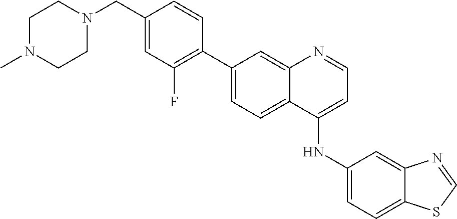 [SMILES: CN1CCN(Cc2ccc(-c3ccc4c(Nc5ccc6scnc6c5)ccnc4c3)c(F)c2)CC1] | N-(7-(2-fluoro-4-((4- methylpiperazin-1- yl)methyl)phenyl)quinolin- 4-yl)benzo[d]thiazol-5- amine |
| 30 | 30 | 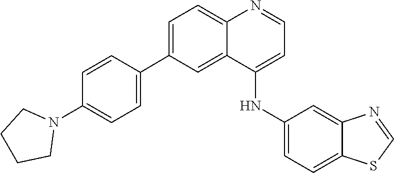 [SMILES: c1cc(Nc2ccc3scnc3c2)c2cc(-c3ccc(N4CCCC4)cc3)ccc2n1] | N-(6-(4-(pyrrolidin-1- yl)phenyl)quinolin-4- yl)benzo[d]thiazol-5- amine |
| 31 | 31 | 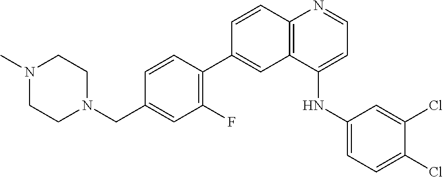 [SMILES: CN1CCN(Cc2ccc(-c3ccc4nccc(Nc5ccc(Cl)c(Cl)c5)c4c3)c(F)c2)CC1] | N-(3,4-dichlorophenyl)-6- (2-fluoro-4-((4- methylpiperazin-1- yl)methyl)phenyl)quinolin- 4-amine |
| 32 | 32 | 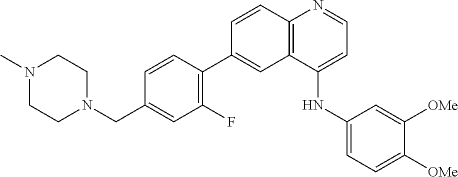 [SMILES: COc1ccc(Nc2ccnc3ccc(-c4ccc(CN5CCN(C)CC5)cc4F)cc23)cc1OC] | N-(3,4-dimethoxyphenyl)- 6-(2-fluoro-4-((4- methylpiperazin-1- yl)methyl)phenyl)quinolin- 4-amine |
| 33 | 33 | 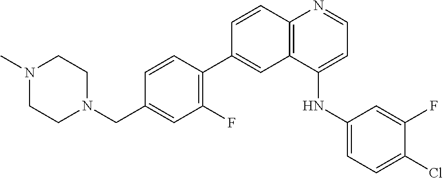 [SMILES: CN1CCN(Cc2ccc(-c3ccc4nccc(Nc5ccc(Cl)c(F)c5)c4c3)c(F)c2)CC1] | N-(4-chloro-3- fluorophenyl)-6-(2-fluoro- 4-((4-methylpiperazin-1- yl)methyl)phenyl)quinolin- 4-amine |
| 34 | 34 | 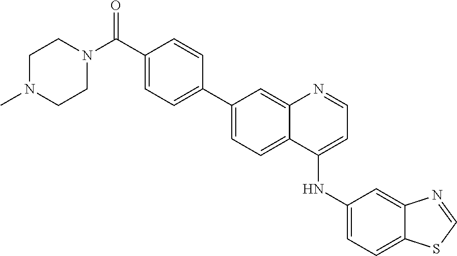 [SMILES: CN1CCN(C(=O)c2ccc(-c3ccc4c(Nc5ccc6scnc6c5)ccnc4c3)cc2)CC1] | (4-(4-(benzo[d]thiazol-5- ylamino)quinolin-7- yl)phenyl)(4- methylpiperazin-1- yl)methanone |
| 35 | 35 | 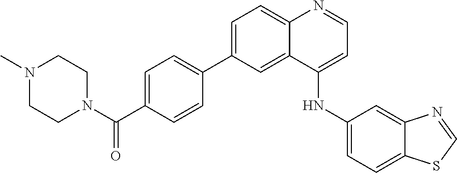 [SMILES: CN1CCN(C(=O)c2ccc(-c3ccc4nccc(Nc5ccc6scnc6c5)c4c3)cc2)CC1] | (4-(4-(benzo[d]thiazol-5- ylamino)quinolin-6- yl)phenyl)(4- methylpiperazin-1- yl)methanone |
| 36 | 36 | 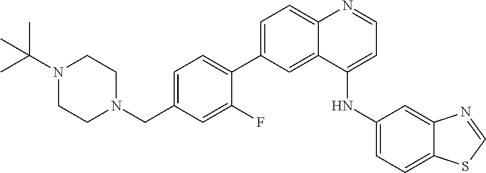 [SMILES: CC(C)(C)N1CCN(Cc2ccc(-c3ccc4nccc(Nc5ccc6scnc6c5)c4c3)c(F)c2)CC1] | N-(6-(4-((4-(tert- butyl)piperazin-1- yl)methyl)-2- fluorophenyl)quinolin-4- yl)benzo[d]thiazol-5- amine |
| 37 | 37 | 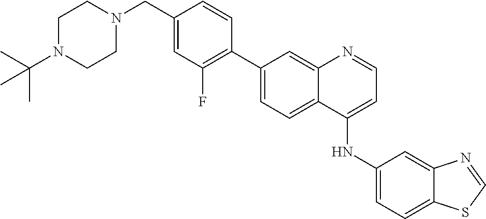 [SMILES: CC(C)(C)N1CCN(Cc2ccc(-c3ccc4c(Nc5ccc6scnc6c5)ccnc4c3)c(F)c2)CC1] | N-(7-(4-((4-(tert- butyl)piperazin-1- yl)methyl)-2- fluorophenyl)quinolin-4- yl)benzo[d]thiazol-5- amine |
| 38 | 38 | 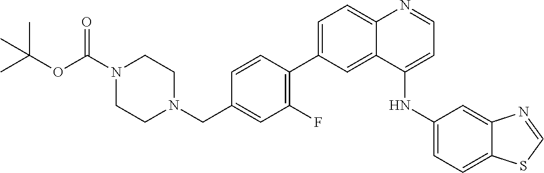 [SMILES: CC(C)(C)OC(=O)N1CCN(Cc2ccc(-c3ccc4nccc(Nc5ccc6scnc6c5)c4c3)c(F)c2)CC1] | tert-butyl 4-(4-(4- (benzo[d]thiazol-5- ylamino)quinolin-6-yl)-3- fluorobenzyl)piperazine-1- carboxylate |
| 39 | 39 | 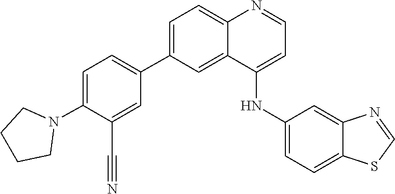 [SMILES: N#Cc1cc(-c2ccc3nccc(Nc4ccc5scnc5c4)c3c2)ccc1N1CCCC1] | 5-(4-(benzo[d]thiazol-5- ylamino)quinolin-6-yl)-2- (pyrrolidin-1- yl)benzonitrile |
| 40 | 40 | 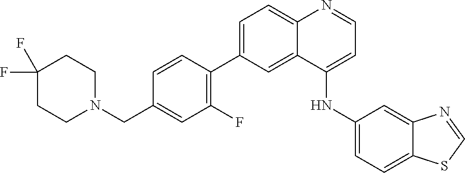 [SMILES: Fc1cc(CN2CCC(F)(F)CC2)ccc1-c1ccc2nccc(Nc3ccc4scnc4c3)c2c1] | N-(6-(4-((4,4- difluoropiperidin-1- yl)methyl)-2- fluorophenyl)quinolin-4- yl)benzo[d]thiazol-5- amine |
| 41 | 41 | 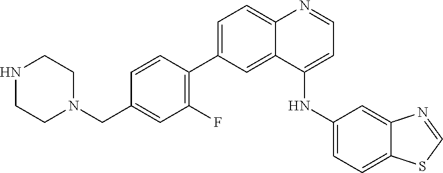 [SMILES: Fc1cc(CN2CCNCC2)ccc1-c1ccc2nccc(Nc3ccc4scnc4c3)c2c1] | N-(6-(2-fluoro-4- (piperazin-1- ylmethyl)phenyl)quinolin- 4-yl)benzo[d]thiazol-5- amine |
| 42 | 42 | 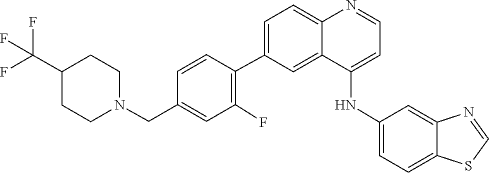 [SMILES: Fc1cc(CN2CCC(C(F)(F)F)CC2)ccc1-c1ccc2nccc(Nc3ccc4scnc4c3)c2c1] | N-(6-(2-fluoro-4-((4- (trifluoromethyl)piperidin- 1- yl)methyl)phenyl)quinolin- 4-yl)benzo[d]thiazol-5- amine |
| 43 | 43 | 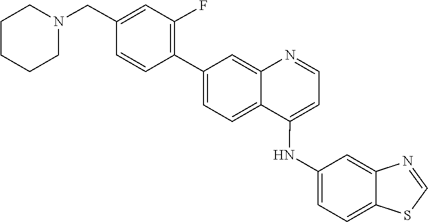 [SMILES: Fc1cc(CN2CCCCC2)ccc1-c1ccc2c(Nc3ccc4scnc4c3)ccnc2c1] | N-(7-(2-fluoro-4- (piperidin-1- ylmethyl)phenyl)quinolin- 4-yl)benzo[d]thiazol-5- amine |
| 44 | 44 | 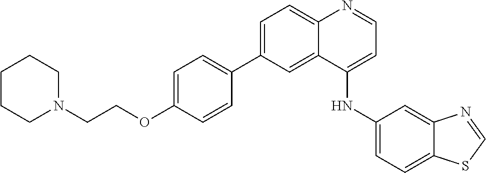 [SMILES: c1cc(Nc2ccc3scnc3c2)c2cc(-c3ccc(OCCN4CCCCC4)cc3)ccc2n1] | N-(6-(4-(2-(piperidin-1- yl)ethoxy)phenyl)quinolin- 4-yl)benzo[d]thiazol-5- amine |
| 47 | 45 | 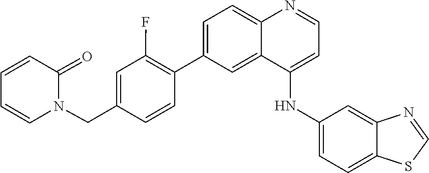 [SMILES: O=c1ccccn1Cc1ccc(-c2ccc3nccc(Nc4ccc5scnc5c4)c3c2)c(F)c1] | 1-(4-(4-(benzo[d]thiazol- 5-ylamino)quinolin-6-yl)- 3-fluorobenzyl)pyridin- 2(1H)-one |
| 50 | 46 | 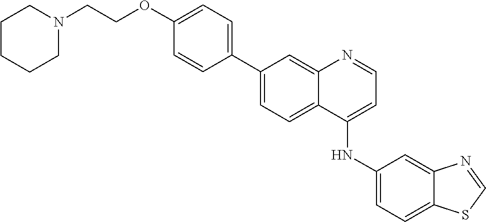 [SMILES: c1cc(Nc2ccc3scnc3c2)c2ccc(-c3ccc(OCCN4CCCCC4)cc3)cc2n1] | N-(7-(4-(2-(piperidin-1- yl)ethoxy)phenyl)quinolin- 4-yl)benzo[d]thiazol-5- amine |
| 51 | 47 | 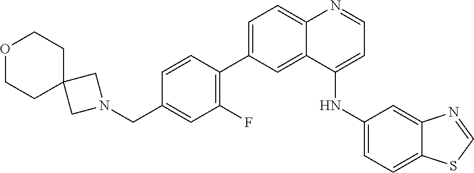 [SMILES: Fc1cc(CN2CC3(CCOCC3)C2)ccc1-c1ccc2nccc(Nc3ccc4scnc4c3)c2c1] | N-(6-(4-((7-oxa-2- azaspiro[3.5]nonan-2- yl)methyl)-2- fluorophenyl)quinolin-4- yl)benzo[d]thiazol-5- amine |
| 52 | 48 | 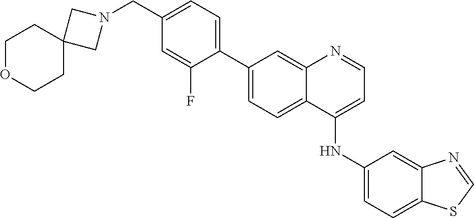 [SMILES: Fc1cc(CN2CC3(CCOCC3)C2)ccc1-c1ccc2c(Nc3ccc4scnc4c3)ccnc2c1] | N-(7-(4-((7-oxa-2- azaspiro[3.5]nonan-2- yl)methyl)-2- fluorophenyl)quinolin-4- yl)benzo[d]thiazol-5- amine |
| 53 (also referred to as Com- pound 99) | 49 | 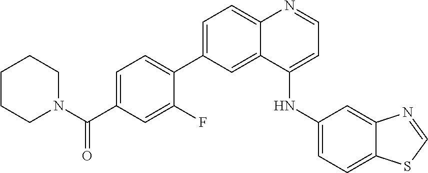 [SMILES: O=C(c1ccc(-c2ccc3nccc(Nc4ccc5scnc5c4)c3c2)c(F)c1)N1CCCCC1] | (4-(4-(benzo[d]thiazol-5- ylamino)quinolin-6-yl)-3- fluorophenyl)(piperidin-1- yl)methanone |
| 54 | 50 | 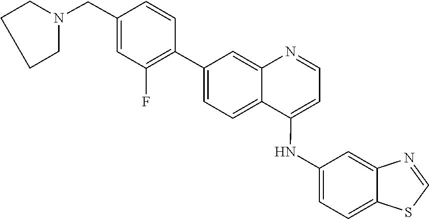 [SMILES: Fc1cc(CN2CCCC2)ccc1-c1ccc2c(Nc3ccc4scnc4c3)ccnc2c1] | N-(7-(2-fluoro-4- (pyrrolidin-1- ylmethyl)phenyl)quinolin- 4-yl)benzo[d]thiazol-5- amine |
| 55 | 51 | 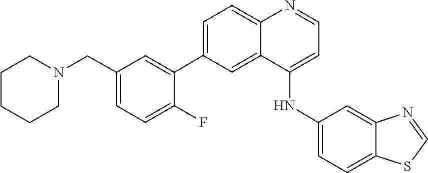 [SMILES: Fc1ccc(CN2CCCCC2)cc1-c1ccc2nccc(Nc3ccc4scnc4c3)c2c1] | N-(6-(2-fluoro-5- (piperidin-1- ylmethyl)phenyl)quinolin- 4-yl)benzo[d]thiazol-5- amine |
| 56 | 52 | 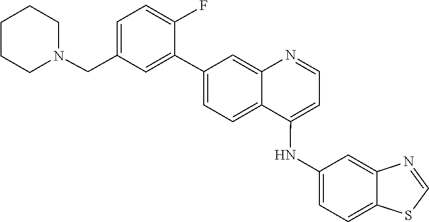 [SMILES: Fc1ccc(CN2CCCCC2)cc1-c1ccc2c(Nc3ccc4scnc4c3)ccnc2c1] | N-(7-(2-fluoro-5- (piperidin-1- ylmethyl)phenyl)quinolin- 4-yl)benzo[d]thiazol-5- amine |
| 57 | 53 | 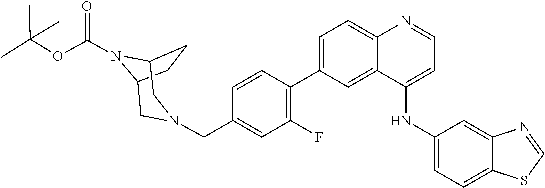 [SMILES: CC(C)(C)OC(=O)N1C2CCC1CN(Cc1ccc(-c3ccc4nccc(Nc5ccc6scnc6c5)c4c3)c(F)c1)C2] | tert-butyl (1R,5S)-3-(4-(4- (benzo[d]thiazol-5- ylamino)quinolin-6-yl)-3- fluorobenzyl)-3,8- diazabicyclo[3.2.1]octane- 8-carboxylate |
| 58 | 54 | 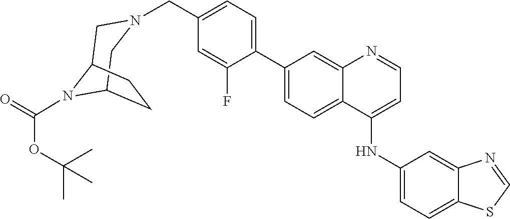 [SMILES: CC(C)(C)OC(=O)N1C2CCC1CN(Cc1ccc(-c3ccc4c(Nc5ccc6scnc6c5)ccnc4c3)c(F)c1)C2] | tert-butyl (1R,5S)-3-(4-(4- (benzo[d]thiazol-5- ylamino)quinolin-7-yl)-3- fluorobenzyl)-3,8- diazabicyclo[3.2.1]octane- 8-carboxylate |
| 61 | 55 | 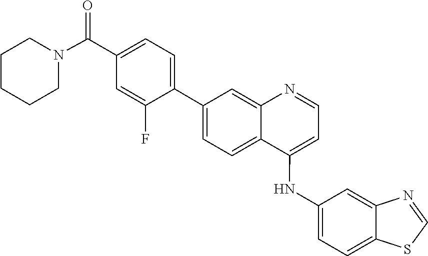 [SMILES: O=C(c1ccc(-c2ccc3c(Nc4ccc5scnc5c4)ccnc3c2)c(F)c1)N1CCCCC1] | (4-(4-(benzo[d]thiazol-5- ylamino)quinolin-7-yl)-3- fluorophenyl)(piperidin-1- yl)methanone |
| 62 | | 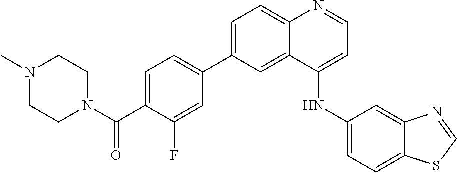 [SMILES: CN1CCN(C(=O)c2ccc(-c3ccc4nccc(Nc5ccc6scnc6c5)c4c3)cc2F)CC1] | (4-(4-(benzo[d]thiazol-5- ylamino)quinolin-6-yl)-2- fluorophenyl)(4- methylpiperazin-1- yl)methanone |
| 63 | | 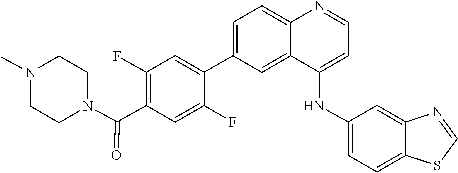 [SMILES: CN1CCN(C(=O)c2cc(F)c(-c3ccc4nccc(Nc5ccc6scnc6c5)c4c3)cc2F)CC1] | (4-(4-(benzo[d]thiazol-5- ylamino)quinolin-6-yl)- 2,5-difluorophenyl)(4- methylpiperazin-1- yl)methanone |
| 64 | | 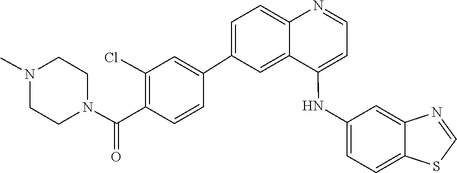 [SMILES: CN1CCN(C(=O)c2ccc(-c3ccc4nccc(Nc5ccc6scnc6c5)c4c3)cc2Cl)CC1] | (4-(4-(benzo[d]thiazol-5- ylamino)quinolin-6-yl)-2- chlorophenyl)(4- methylpiperazin-1- yl)methanone |
| 65 | | 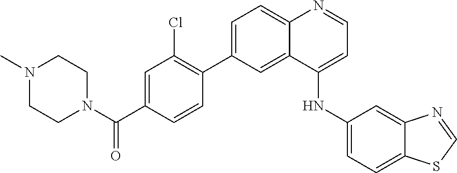 [SMILES: CN1CCN(C(=O)c2ccc(-c3ccc4nccc(Nc5ccc6scnc6c5)c4c3)c(Cl)c2)CC1] | (4-(4-(benzo[d]thiazol-5- ylamino)quinolin-6-yl)-3- chlorophenyl)(4- methylpiperazin-1- yl)methanone |
| 66 | | 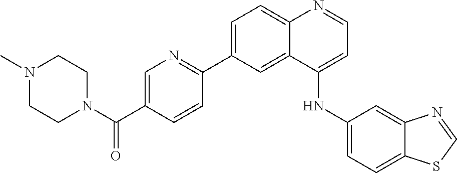 [SMILES: CN1CCN(C(=O)c2ccc(-c3ccc4nccc(Nc5ccc6scnc6c5)c4c3)nc2)CC1] | (6-(4-(benzo[d]thiazol-5- ylamino)quinolin-6- yl)pyridin-3-yl)(4- methylpiperazin-1- yl)methanone |
| 67 | | 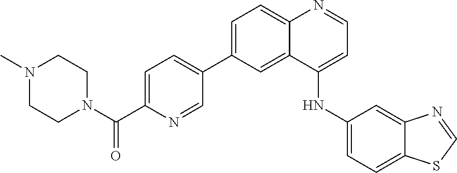 [SMILES: CN1CCN(C(=O)c2ccc(-c3ccc4nccc(Nc5ccc6scnc6c5)c4c3)cn2)CC1] | (5-(4-(benzo[d]thiazol-5- ylamino)quinolin-6- yl)pyridin-2-yl)(4- methylpiperazin-1- yl)methanone |
| 68 | | 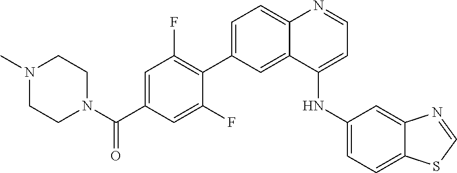 [SMILES: CN1CCN(C(=O)c2cc(F)c(-c3ccc4nccc(Nc5ccc6scnc6c5)c4c3)c(F)c2)CC1] | (4-(4-(benzo[d]thiazol-5- ylamino)quinolin-6-yl)- 3,5-difluorophenyl)(4- methylpiperazin-1- yl)methanone |
| 69 | | 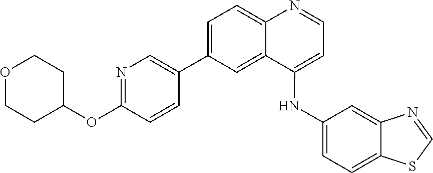 [SMILES: c1cc(Nc2ccc3scnc3c2)c2cc(-c3ccc(OC4CCOCC4)nc3)ccc2n1] | N-(6-(6-((tetrahydro-2H- pyran-4-yl)oxy)pyridin-3- yl)quinolin-4- yl)benzo[d]thiazol-5- amine |
| 70 | | 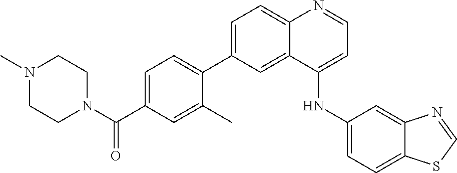 [SMILES: Cc1cc(C(=O)N2CCN(C)CC2)ccc1-c1ccc2nccc(Nc3ccc4scnc4c3)c2c1] | (4-(4-(benzo[d]thiazol-5- ylamino)quinolin-6-yl)-3- methylphenyl)(4- methylpiperazin-1- yl)methanone |
| 71 | | 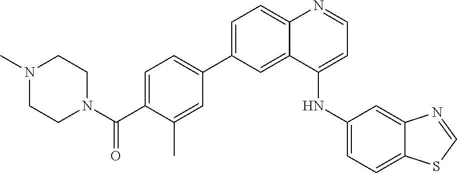 [SMILES: Cc1cc(-c2ccc3nccc(Nc4ccc5scnc5c4)c3c2)ccc1C(=O)N1CCN(C)CC1] | (4-(4-(benzo[d]thiazol-5- ylamino)quinolin-6-yl)-2- methylphenyl)(4- methylpiperazin-1- yl)methanone |
| 72 | | 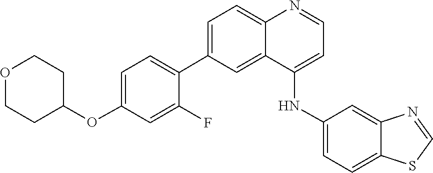 [SMILES: Fc1cc(OC2CCOCC2)ccc1-c1ccc2nccc(Nc3ccc4scnc4c3)c2c1] | N-(6-(2-fluoro-4- ((tetrahydro-2H-pyran-4- yl)oxy)phenyl)quinolin-4- yl)benzo[d]thiazol-5- amine |
| 73 | | 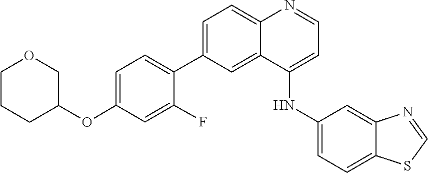 [SMILES: Fc1cc(OC2CCCOC2)ccc1-c1ccc2nccc(Nc3ccc4scnc4c3)c2c1] | N-(6-(2-fluoro-4- ((tetrahydro-2H-pyran-3- yl)oxy)phenyl)quinolin-4- yl)benzo[d]thiazol-5- amine |
| 74 (also referred to as Com- pound 254 and Com- pound 295) | 95 | 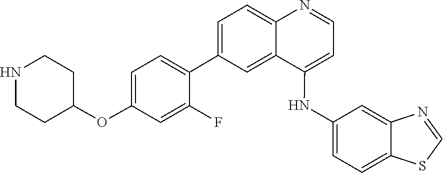 [SMILES: Fc1cc(OC2CCNCC2)ccc1-c1ccc2nccc(Nc3ccc4scnc4c3)c2c1] | N-(6-(2-fluoro-4- (piperidin-4- yloxy)phenyl)quinolin-4- yl)benzo[d]thiazol-5- amine |
| 75 (also referred to as Com- pound 224) | | 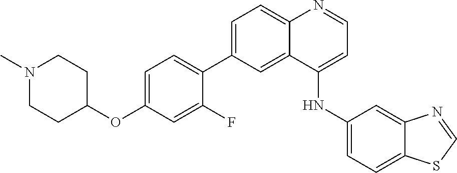 [SMILES: CN1CCC(Oc2ccc(-c3ccc4nccc(Nc5ccc6scnc6c5)c4c3)c(F)c2)CC1] | N-(6-(2-fluoro-4-((1- methylpiperidin-4- yl)oxy)phenyl)quinolin-4- yl)benzo[d]thiazol-5- amine |
| 76 | | 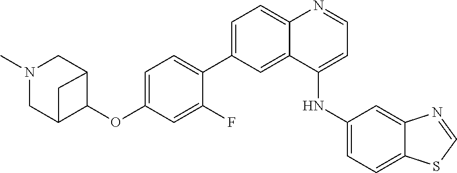 [SMILES: CN1CC2CC(C1)C2Oc1ccc(-c2ccc3nccc(Nc4ccc5scnc5c4)c3c2)c(F)c1] | N-(6-(2-fluoro-4-((3- methyl-3- azabicyclo[3.1.1]heptan-6- yl)oxy)phenyl)quinolin-4- yl)benzo[d]thiazol-5- amine |
| 77 | | 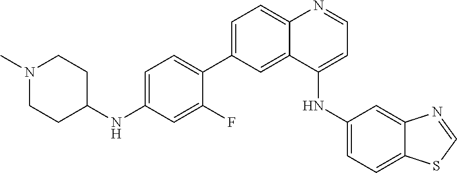 [SMILES: CN1CCC(Nc2ccc(-c3ccc4nccc(Nc5ccc6scnc6c5)c4c3)c(F)c2)CC1] | N-(6-(2-fluoro-4-((1- methylpiperidin-4- yl)amino)phenyl)quinolin- 4-yl)benzo[d]thiazol-5- amine |
| 79 (also referred to as Com- pound 273) | 67 | 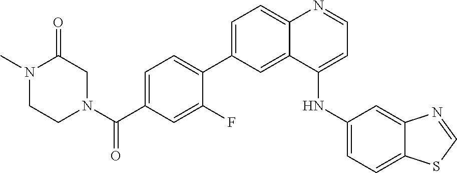 [SMILES: CN1CCN(C(=O)c2ccc(-c3ccc4nccc(Nc5ccc6scnc6c5)c4c3)c(F)c2)CC1=O] | 4-(4-(4-(benzo[d]thiazol- 5-ylamino)quinolin-6-yl)- 3-fluorobenzoyl)-1- methylpiperazin-2-one |
| 81 | | 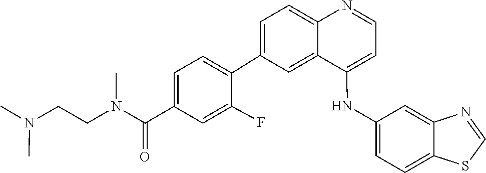 [SMILES: CN(C)CCN(C)C(=O)c1ccc(-c2ccc3nccc(Nc4ccc5scnc5c4)c3c2)c(F)c1] | 4-(4-(benzo[d]thiazol-5- ylamino)quinolin-6-yl)-N- (2-(dimethylamino)ethyl)- 3-fluoro-N- methylbenzamide |
| 82 | | 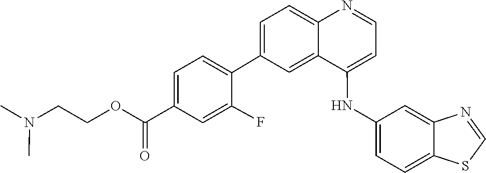 [SMILES: CN(C)CCOC(=O)c1ccc(-c2ccc3nccc(Nc4ccc5scnc5c4)c3c2)c(F)c1] | 2-(dimethylamino)ethyl 4- (4-(benzo[d]thiazol-5- ylamino)quinolin-6-yl)-3- fluorobenzoate |
| 83 | | 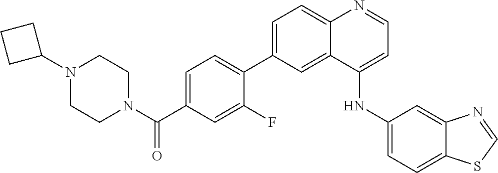 [SMILES: O=C(c1ccc(-c2ccc3nccc(Nc4ccc5scnc5c4)c3c2)c(F)c1)N1CCN(C2CCC2)CC1] | (4-(4-(benzo[d]thiazol-5- ylamino)quinolin-6-yl)-3- fluorophenyl)(4- cyclobutylpiperazin-1- yl)methanone |
| 84 | | 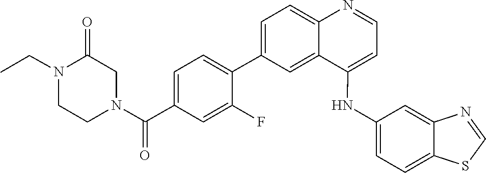 [SMILES: CCN1CCN(C(=O)c2ccc(-c3ccc4nccc(Nc5ccc6scnc6c5)c4c3)c(F)c2)CC1=O] | 4-(4-(4-(benzo[d]thiazol- 5-ylamino)quinolin-6-yl)- 3-fluorobenzoyl)-1- ethylpiperazin-2-one |
| 85 (also referred to as Com- pound 341) | | 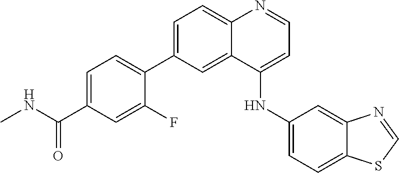 [SMILES: CNC(=O)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N- methylbenzamide |
| 86 | | 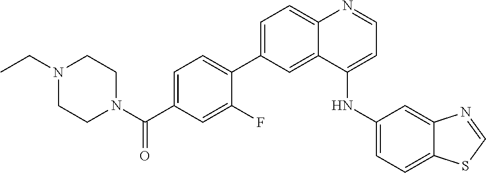 [SMILES: CCN1CCN(C(=O)c2ccc(-c3ccc4nccc(Nc5ccc6scnc6c5)c4c3)c(F)c2)CC1] | (4-(4-(benzo[d]thiazol-5- ylamino)quinolin-6-yl)-3- fluorophenyl)(4- ethylpiperazin-1- yl)methanone |
| 87 | | 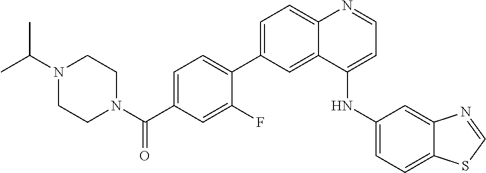 [SMILES: CC(C)N1CCN(C(=O)c2ccc(-c3ccc4nccc(Nc5ccc6scnc6c5)c4c3)c(F)c2)CC1] | (4-(4-(benzo[d]thiazol-5- ylamino)quinolin-6-yl)-3- fluorophenyl)(4- isopropylpiperazin-1- yl)methanone |
| 88 | | 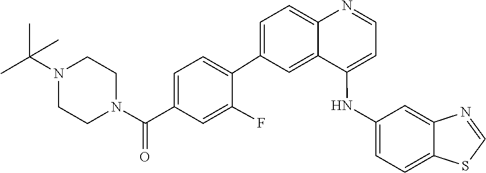 [SMILES: CC(C)(C)N1CCN(C(=O)c2ccc(-c3ccc4nccc(Nc5ccc6scnc6c5)c4c3)c(F)c2)CC1] | (4-(4-(benzo[d]thiazol-5- ylamino)quinolin-6-yl)-3- fluorophenyl)(4-(tert- butyl)piperazin-1- yl)methanone |
| 89 | | 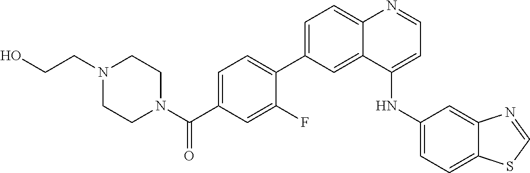 [SMILES: O=C(c1ccc(-c2ccc3nccc(Nc4ccc5scnc5c4)c3c2)c(F)c1)N1CCN(CCO)CC1] | (4-(4-(benzo[d]thiazol-5- ylamino)quinolin-6-yl)-3- fluorophenyl)(4-(2- hydroxyethyl)piperazin-1- yl)methanone |
| 90 | | 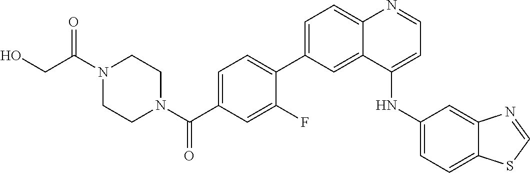 [SMILES: O=C(CO)N1CCN(C(=O)c2ccc(-c3ccc4nccc(Nc5ccc6scnc6c5)c4c3)c(F)c2)CC1] | 1-(4-(4-(4- (benzo[d]thiazol-5- ylamino)quinolin-6-yl)-3- fluorobenzoyl)piperazin-1- yl)-2-hydroxyethan-1-one |
| 91 | | 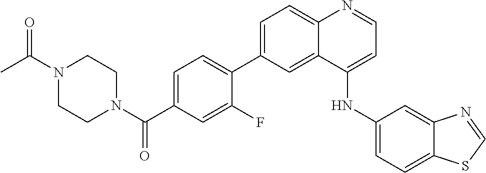 [SMILES: CC(=O)N1CCN(C(=O)c2ccc(-c3ccc4nccc(Nc5ccc6scnc6c5)c4c3)c(F)c2)CC1] | 1-(4-(4-(4- (benzo[d]thiazol-5- ylamino)quinolin-6-yl)-3- fluorobenzoyl)piperazin-1- yl)ethan-1-one |
| 92 | | 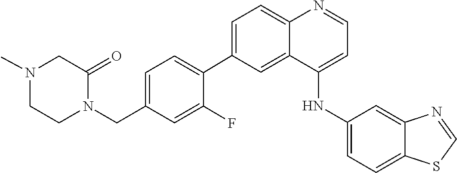 [SMILES: CN1CCN(Cc2ccc(-c3ccc4nccc(Nc5ccc6scnc6c5)c4c3)c(F)c2)C(=O)C1] | 1-(4-(4-(benzo[d]thiazol- 5-ylamino)quinolin-6-yl)- 3-fluorobenzyl)-4- methylpiperazin-2-one |
| 93 | | 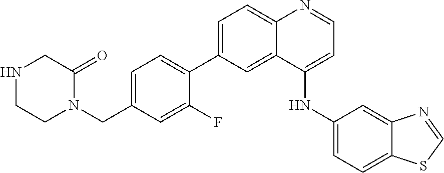 [SMILES: O=C1CNCCN1Cc1ccc(-c2ccc3nccc(Nc4ccc5scnc5c4)c3c2)c(F)c1] | 1-(4-(4-(benzo[d]thiazol- 5-ylamino)quinolin-6-yl)- 3-fluorobenzyl)piperazin- 2-one |
| 94 | | 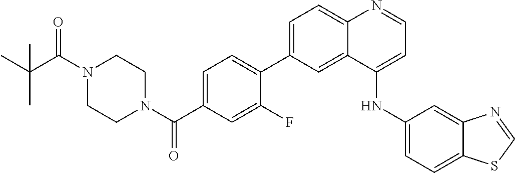 [SMILES: CC(C)(C)C(=O)N1CCN(C(=O)c2ccc(-c3ccc4nccc(Nc5ccc6scnc6c5)c4c3)c(F)c2)CC1] | 1-(4-(4-(4- (benzo[d]thiazol-5- ylamino)quinolin-6-yl)-3- fluorobenzoyl)piperazin-1- yl)-2,2-dimethylpropan-1- one |
| 95 | | 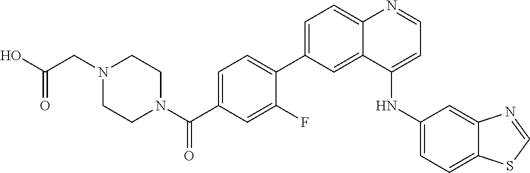 [SMILES: O=C(O)CN1CCN(C(=O)c2ccc(-c3ccc4nccc(Nc5ccc6scnc6c5)c4c3)c(F)c2)CC1] | 2-(4-(4-(4- (benzo[d]thiazol-5- ylamino)quinolin-6-yl)-3- fluorobenzoyl)piperazin-1- yl)acetic acid |
| 96 | | 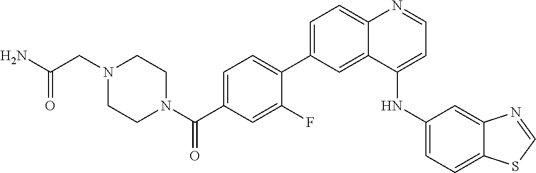 [SMILES: NC(=O)CN1CCN(C(=O)c2ccc(-c3ccc4nccc(Nc5ccc6scnc6c5)c4c3)c(F)c2)CC1] | 2-(4-(4-(4- (benzo[d]thiazol-5- ylamino)quinolin-6-yl)-3- fluorobenzoyl)piperazin-1- yl)acetamide |
| 97 | | 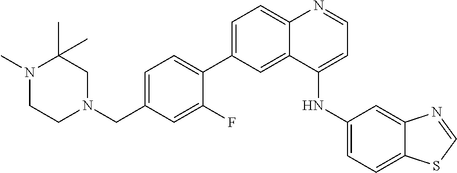 [SMILES: CN1CCN(Cc2ccc(-c3ccc4nccc(Nc5ccc6scnc6c5)c4c3)c(F)c2)CC1(C)C] | N-(6-(2-fluoro-4-((3,3,4- trimethylpiperazin-1- yl)methyl)phenyl)quinolin- 4-yl)benzo[d]thiazol-5- amine |
| 98 | | 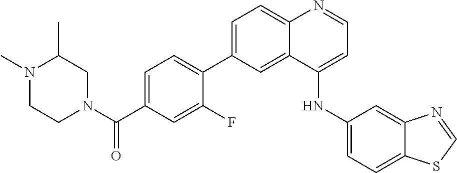 [SMILES: CC1CN(C(=O)c2ccc(-c3ccc4nccc(Nc5ccc6scnc6c5)c4c3)c(F)c2)CCN1C] | (4-(4-(benzo[d]thiazol-5- ylamino)quinolin-6-yl)-3- fluorophenyl)(3,4- dimethylpiperazin-1- yl)methanone |
| 99 (also referred to as Com- pound 53) | 49 | 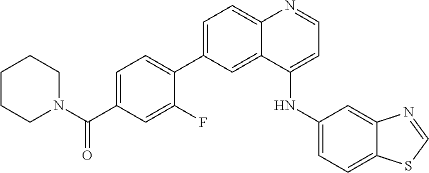 [SMILES: O=C(c1ccc(-c2ccc3nccc(Nc4ccc5scnc5c4)c3c2)c(F)c1)N1CCCCC1] | (4-(4-(benzo[d]thiazol-5- ylamino)quinolin-6-yl)-3- fluorophenyl)(piperidin-1- yl)methanone |
| 100 (also referred to as Com- pound 241) | 58 | 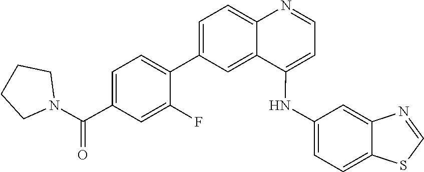 [SMILES: O=C(c1ccc(-c2ccc3nccc(Nc4ccc5scnc5c4)c3c2)c(F)c1)N1CCCC1] | (4-(4-(benzo[d]thiazol-5- ylamino)quinolin-6-yl)-3- fluorophenyl)(pyrrolidin- 1-yl)methanone |
| 101 (also referred to as Com- pound 105) | | 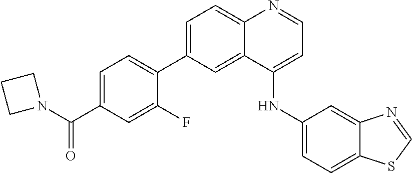 [SMILES: O=C(c1ccc(-c2ccc3nccc(Nc4ccc5scnc5c4)c3c2)c(F)c1)N1CCC1] | azetidin-1-yl(4-(4- (benzo[d]thiazol-5- ylamino)quinolin-6-yl)-3- fluorophenyl)methanone |
| 102 | | 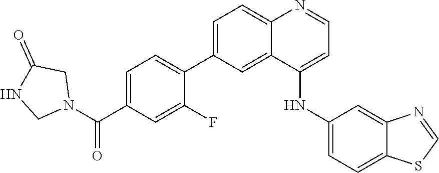 [SMILES: O=C1CN(C(=O)c2ccc(-c3ccc4nccc(Nc5ccc6scnc6c5)c4c3)c(F)c2)CN1] | 1-(4-(4-(benzo[d]thiazol- 5-ylamino)quinolin-6-yl)- 3-fluorobenzoyl) imidazolidin- 4-one |
| 103 | | 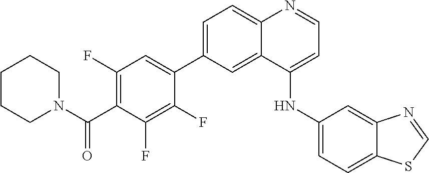 [SMILES: O=C(c1c(F)cc(-c2ccc3nccc(Nc4ccc5scnc5c4)c3c2)c(F)c1F)N1CCCCC1] | (4-(4-(benzo[d]thiazol-5- ylamino)quinolin-6-yl)- 2,3,6- trifluorophenyl)(piperidin- 1-yl)methanone |
| 104 (also referred to as Com- pound 243) | 60 | 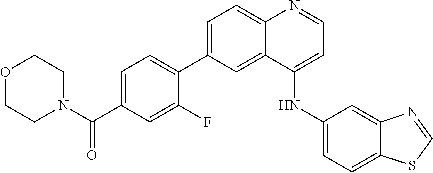 [SMILES: O=C(c1ccc(-c2ccc3nccc(Nc4ccc5scnc5c4)c3c2)c(F)c1)N1CCOCC1] | (4-(4-(benzo[d]thiazol-5- ylamino)quinolin-6-yl)-3- fluorophenyl)(morpholino) methanone |
| 105 (also referred to as Com- pound 101) | | 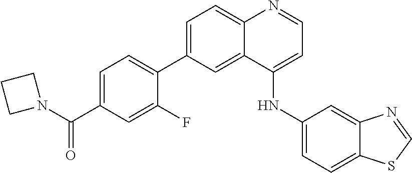 [SMILES: O=C(c1ccc(-c2ccc3nccc(Nc4ccc5scnc5c4)c3c2)c(F)c1)N1CCC1] | azetidin-1-yl(4-(4- (benzo[d]thiazol-5- ylamino)quinolin-6-yl)-3- fluorophenyl)methanone |
| 106 | | 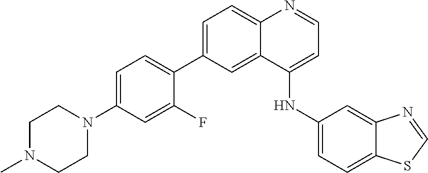 [SMILES: CN1CCN(c2ccc(-c3ccc4nccc(Nc5ccc6scnc6c5)c4c3)c(F)c2)CC1] | N-(6-(2-fluoro-4-(4- methylpiperazin-1- yl)phenyl)quinolin-4- yl)benzo[d]thiazol-5- amine |
| 107 | | 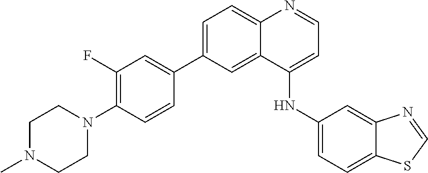 [SMILES: CN1CCN(c2ccc(-c3ccc4nccc(Nc5ccc6scnc6c5)c4c3)cc2F)CC1] | N-(6-(3-fluoro-4-(4- methylpiperazin-1- yl)phenyl)quinolin-4- yl)benzo[d]thiazol-5- amine |
| 108 | | 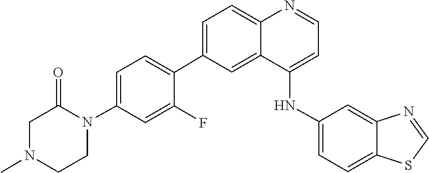 [SMILES: CN1CCN(c2ccc(-c3ccc4nccc(Nc5ccc6scnc6c5)c4c3)c(F)c2)C(=O)C1] | 1-(4-(4-(benzo[d]thiazol- 5-ylamino)quinolin-6-yl)- 3-fluorophenyl)-4- methylpiperazin-2-one |
| 109 | | 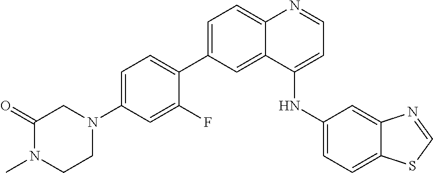 [SMILES: CN1CCN(c2ccc(-c3ccc4nccc(Nc5ccc6scnc6c5)c4c3)c(F)c2)CC1=O] | 4-(4-(4-(benzo[d]thiazol- 5-ylamino)quinolin-6-yl)- 3-fluorophenyl)-1- methylpiperazin-2-one |
| 110 | | 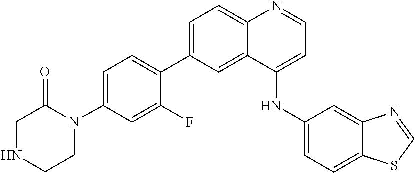 [SMILES: O=C1CNCCN1c1ccc(-c2ccc3nccc(Nc4ccc5scnc5c4)c3c2)c(F)c1] | 1-(4-(4-(benzo[d]thiazol- 5-ylamino)quinolin-6-yl)- 3-fluorophenyl)piperazin- 2-one |
| 111 | | 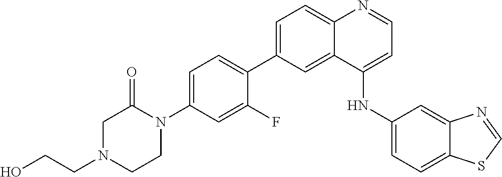 [SMILES: O=C1CN(CCO)CCN1c1ccc(-c2ccc3nccc(Nc4ccc5scnc5c4)c3c2)c(F)c1] | 1-(4-(4-(benzo[d]thiazol- 5-ylamino)quinolin-6-yl)- 3-fluorophenyl)-4-(2- hydroxyethyl)piperazin-2- one |
| 112 | | 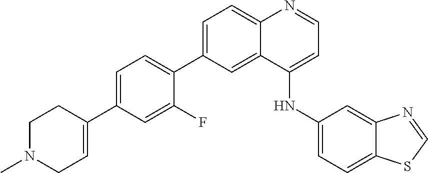 [SMILES: CN1CC=C(c2ccc(-c3ccc4nccc(Nc5ccc6scnc6c5)c4c3)c(F)c2)CC1] | N-(6-(2-fluoro-4-(1- methyl-1,2,3,6- tetrahydropyridin-4- yl)phenyl)quinolin-4- yl)benzo[d]thiazol-5- amine |
| 113 | | 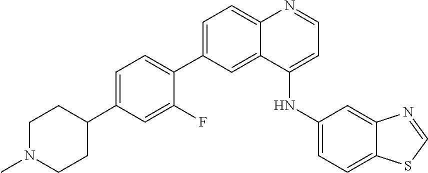 [SMILES: CN1CCC(c2ccc(-c3ccc4nccc(Nc5ccc6scnc6c5)c4c3)c(F)c2)CC1] | N-(6-(2-fluoro-4-(1- methylpiperidin-4- yl)phenyl)quinolin-4- yl)benzo[d]thiazol-5- amine |
| 114 | | 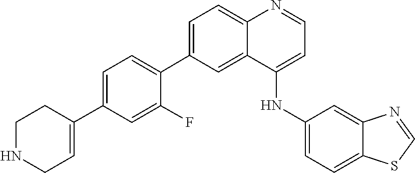 [SMILES: Fc1cc(C2=CCNCC2)ccc1-c1ccc2nccc(Nc3ccc4scnc4c3)c2c1] | N-(6-(2-fluoro-4-(1,2,3,6- tetrahydropyridin-4- yl)phenyl)quinolin-4- yl)benzo[d]thiazol-5- amine |
| 115 | | 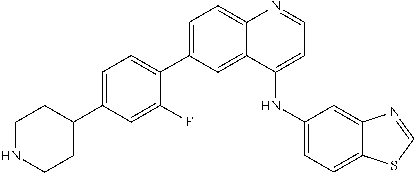 [SMILES: Fc1cc(C2CCNCC2)ccc1-c1ccc2nccc(Nc3ccc4scnc4c3)c2c1] | N-(6-(2-fluoro-4- (piperidin-4- yl)phenyl)quinolin-4- yl)benzo[d]thiazol-5- amine |
| 116 | | 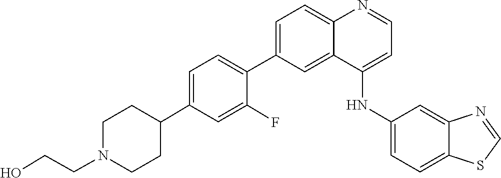 [SMILES: OCCN1CCC(c2ccc(-c3ccc4nccc(Nc5ccc6scnc6c5)c4c3)c(F)c2)CC1] | 2-(4-(4-(4- (benzo[d]thiazol-5- ylamino)quinolin-6-yl)-3- fluorophenyl)piperidin-1- yl)ethan-1-ol |
| 117 | | 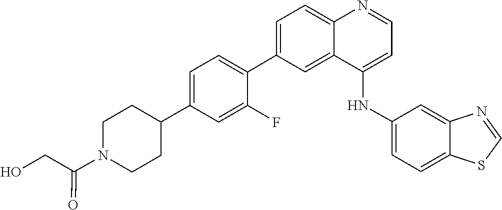 [SMILES: O=C(CO)N1CCC(c2ccc(-c3ccc4nccc(Nc5ccc6scnc6c5)c4c3)c(F)c2)CC1] | 1-(4-(4-(4- (benzo[d]thiazol-5- ylamino)quinolin-6-yl)-3- fluorophenyl)piperidin-1- yl)-2-hydroxyethan-1-one |
| 118 | | 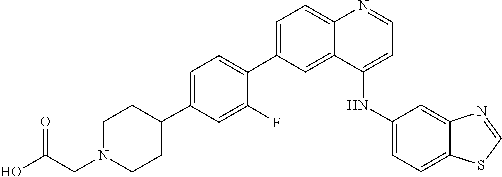 [SMILES: O=C(O)CN1CCC(c2ccc(-c3ccc4nccc(Nc5ccc6scnc6c5)c4c3)c(F)c2)CC1] | 2-(4-(4-(4- (benzo[d]thiazol-5- ylamino)quinolin-6-yl)-3- fluorophenyl)piperidin-1- yl)acetic acid |
| 119 | | 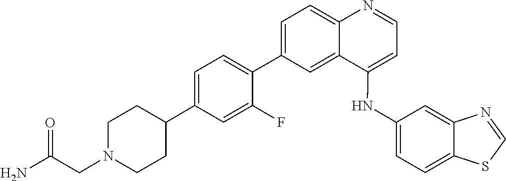 [SMILES: NC(=O)CN1CCC(c2ccc(-c3ccc4nccc(Nc5ccc6scnc6c5)c4c3)c(F)c2)CC1] | 2-(4-(4-(4- (benzo[d]thiazol-5- ylamino)quinolin-6-yl)-3- fluorophenyl)piperidin-1- yl)acetamide |
| 120 | | 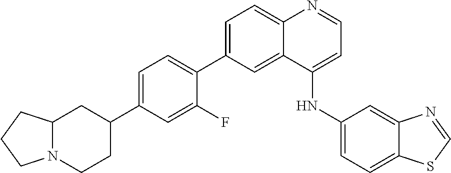 [SMILES: Fc1cc(C2CCN3CCCC3C2)ccc1-c1ccc2nccc(Nc3ccc4scnc4c3)c2c1] | N-(6-(2-fluoro-4- (octahydroindolizin-7- yl)phenyl)quinolin-4- yl)benzo[d]thiazol-5- amine |
| 121 | | 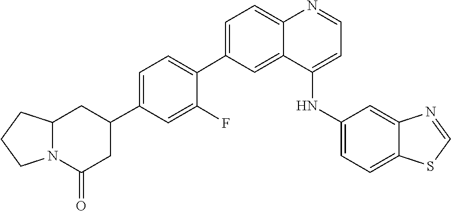 [SMILES: O=C1CC(c2ccc(-c3ccc4nccc(Nc5ccc6scnc6c5)c4c3)c(F)c2)CC2CCCN12] | 7-(4-(4-(benzo[d]thiazol- 5-ylamino)quinolin-6-yl)- 3- fluorophenyl)hexahydro- indolizin-5(1H)-one |
| 122 | | 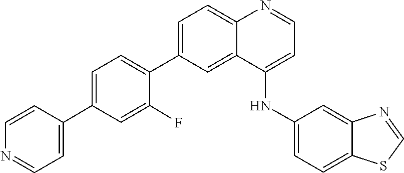 [SMILES: Fc1cc(-c2ccncc2)ccc1-c1ccc2nccc(Nc3ccc4scnc4c3)c2c1] | N-(6-(2-fluoro-4-(pyridin- 4-yl)phenyl)quinolin-4- yl)benzo[d]thiazol-5- amine |
| 123 | | 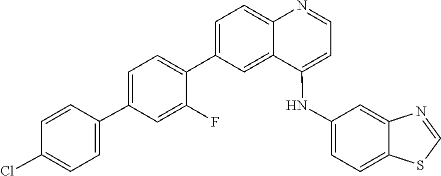 [SMILES: Fc1cc(-c2ccc(Cl)cc2)ccc1-c1ccc2nccc(Nc3ccc4scnc4c3)c2c1] | N-(6-(4′-chloro-3-fluoro- [1,1′-biphenyl]-4- yl)quinolin-4- yl)benzo[d]thiazol-5- amine |
| 124 | | 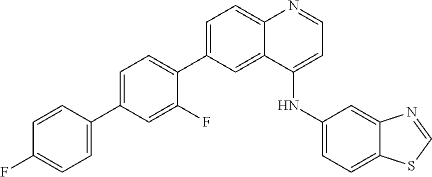 [SMILES: Fc1ccc(-c2ccc(-c3ccc4nccc(Nc5ccc6scnc6c5)c4c3)c(F)c2)cc1] | N-(6-(3,4′-difluoro-[1,1′- biphenyl]-4-yl)quinolin-4- yl)benzo[d]thiazol-5- amine |
| 125 | | 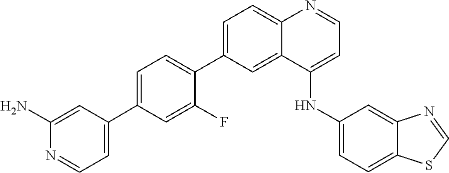 [SMILES: Nc1cc(-c2ccc(-c3ccc4nccc(Nc5ccc6scnc6c5)c4c3)c(F)c2)ccn1] | N-(6-(4-(2-aminopyridin- 4-yl)-2- fluorophenyl)quinolin-4- yl)benzo[d]thiazol-5- amine |
| 126 | | 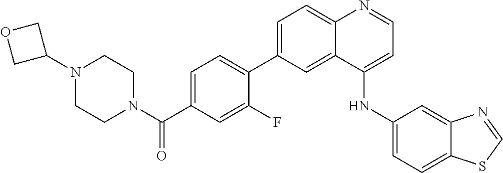 [SMILES: O=C(c1ccc(-c2ccc3nccc(Nc4ccc5scnc5c4)c3c2)c(F)c1)N1CCN(C2COC2)CC1] | (4-(4-(benzo[d]thiazol-5- ylamino)quinolin-6-yl)-3- fluorophenyl)(4-(oxetan-3- yl)piperazin-1- yl)methanone |
| 127 | | 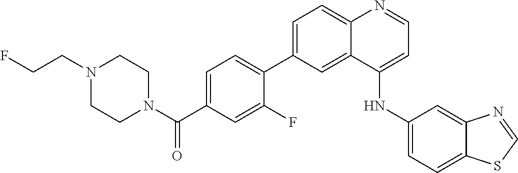 [SMILES: O=C(c1ccc(-c2ccc3nccc(Nc4ccc5scnc5c4)c3c2)c(F)c1)N1CCN(CCF)CC1] | (4-(4-(benzo[d]thiazol-5- ylamino)quinolin-6-yl)-3- fluorophenyl)(4-(2- fluoroethyl)piperazin-1- yl)methanone |
| 128 | | 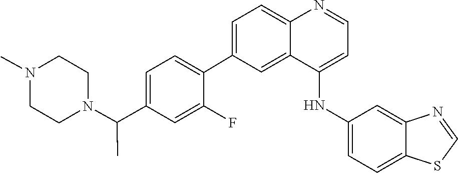 [SMILES: CC(c1ccc(-c2ccc3nccc(Nc4ccc5scnc5c4)c3c2)c(F)c1)N1CCN(C)CC1] | N-(6-(2-fluoro-4-(1-(4- methylpiperazin-1- yl)ethyl)phenyl)quinolin- 4-yl)benzo[d]thiazol-5- amine |
| 129 | | 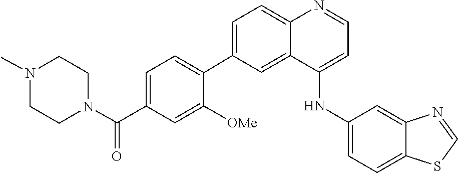 [SMILES: COc1cc(C(=O)N2CCN(C)CC2)ccc1-c1ccc2nccc(Nc3ccc4scnc4c3)c2c1] | (4-(4-(benzo[d]thiazol-5- ylamino)quinolin-6-yl)-3- methoxyphenyl)(4- methylpiperazin-1- yl)methanone |
| 130 | | 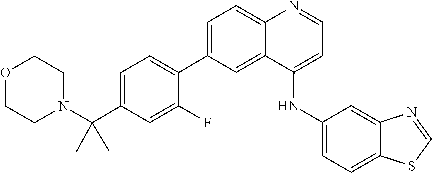 [SMILES: CC(C)(c1ccc(-c2ccc3nccc(Nc4ccc5scnc5c4)c3c2)c(F)c1)N1CCOCC1] | N-(6-(2-fluoro-4-(2- morpholinopropan-2- yl)phenyl)quinolin-4- yl)benzo[d]thiazol-5- amine |
| 131 | | 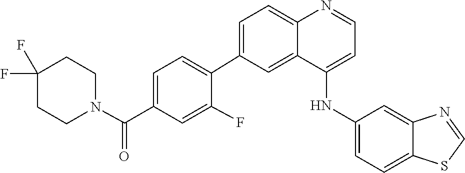 [SMILES: O=C(c1ccc(-c2ccc3nccc(Nc4ccc5scnc5c4)c3c2)c(F)c1)N1CCC(F)(F)CC1] | (4-(4-(benzo[d]thiazol-5- ylamino)quinolin-6-yl)-3- fluorophenyl)(4,4- difluoropiperidin-1- yl)methanone |
| 132 (also referred to as Com- pound 317) | 117 | 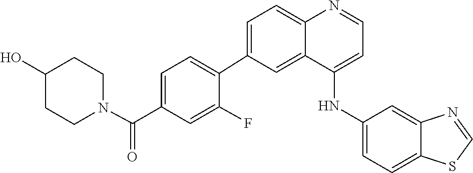 [SMILES: O=C(c1ccc(-c2ccc3nccc(Nc4ccc5scnc5c4)c3c2)c(F)c1)N1CCC(O)CC1] | (4-(4-(benzo[d]thiazol-5- ylamino)quinolin-6-yl)-3- fluorophenyl)(4- hydroxypiperidin-1- yl)methanone |
| 133 | | 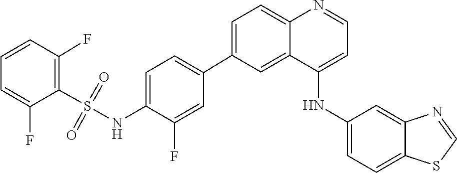 [SMILES: O=S(=O)(Nc1ccc(-c2ccc3nccc(Nc4ccc5scnc5c4)c3c2)cc1F)c1c(F)cccc1F] | N-(4-(4-(benzo[d]thiazol- 5-ylamino)quinolin-6-yl)- 2-fluorophenyl)-2,6- difluorobenzenesulfonamide |
| 134 | | 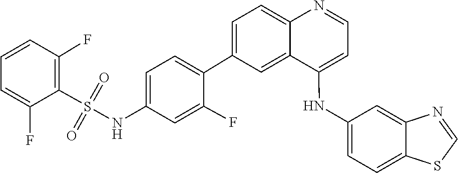 [SMILES: O=S(=O)(Nc1ccc(-c2ccc3nccc(Nc4ccc5scnc5c4)c3c2)c(F)c1)c1c(F)cccc1F] | N-(4-(4-(benzo[d]thiazol- 5-ylamino)quinolin-6-yl)- 3-fluorophenyl)-2,6- difluorobenzenesulfonamide |
| 135 | | 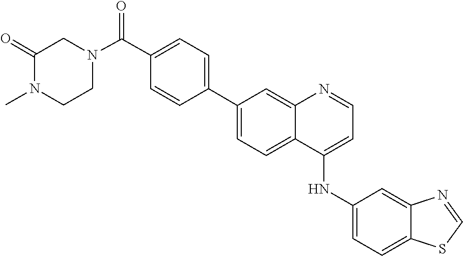 [SMILES: CN1CCN(C(=O)c2ccc(-c3ccc4c(Nc5ccc6scnc6c5)ccnc4c3)cc2)CC1=O] | 4-(4-(4-(benzo[d]thiazol- 5-ylamino)quinolin-7- yl)benzoyl)-1- methylpiperazin-2-one |
| 136 (also referred to as Com- pound 274) | 68 | 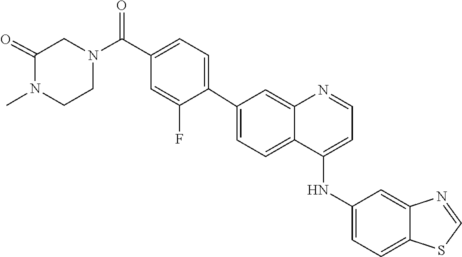 [SMILES: CN1CCN(C(=O)c2ccc(-c3ccc4c(Nc5ccc6scnc6c5)ccnc4c3)c(F)c2)CC1=O] | 4-(4-(4-(benzo[d]thiazol- 5-ylamino)quinolin-7-yl)- 3-fluorobenzoyl)-1- methylpiperazin-2-one |
| 137 | | 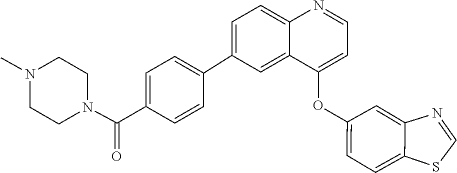 [SMILES: CN1CCN(C(=O)c2ccc(-c3ccc4nccc(Oc5ccc6scnc6c5)c4c3)cc2)CC1] | (4-(4-(benzo[d]thiazol-5- yloxy)quinolin-6- yl)phenyl)(4- methylpiperazin-1- yl)methanone |
| 138 | | 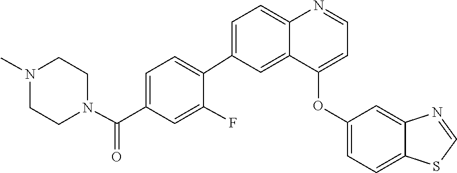 [SMILES: CN1CCN(C(=O)c2ccc(-c3ccc4nccc(Oc5ccc6scnc6c5)c4c3)c(F)c2)CC1] | (4-(4-(benzo[d]thiazol-5- yloxy)quinolin-6-yl)-3- fluorophenyl)(4- methylpiperazin-1- yl)methanone |
| 139 | | 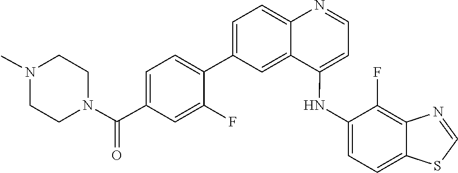 [SMILES: CN1CCN(C(=O)c2ccc(-c3ccc4nccc(Nc5ccc6scnc6c5F)c4c3)c(F)c2)CC1] | (3-fluoro-4-(4-((4- fluorobenzo[d]thiazol-5- yl)amino)quinolin-6- yl)phenyl)(4- methylpiperazin-1- yl)methanone |
| 140 | | 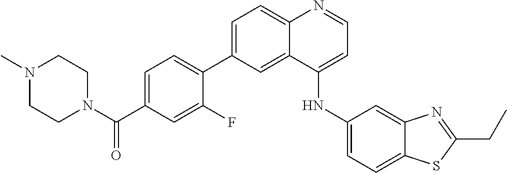 [SMILES: CCc1nc2cc(Nc3ccnc4ccc(-c5ccc(C(=O)N6CCN(C)CC6)cc5F)cc34)ccc2s1] | (4-(4-((2- ethylbenzo[d]thiazol-5- yl)amino)quinolin-6-yl)-3- fluorophenyl)(4- methylpiperazin-1- yl)methanone |
| 141 | | 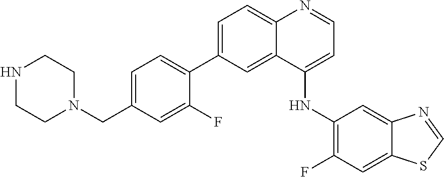 [SMILES: Fc1cc2scnc2cc1Nc1ccnc2ccc(-c3ccc(CN4CCNCC4)cc3F)cc12] | 6-fluoro-N-(6-(2-fluoro-4- (piperazin-1- ylmethyl)phenyl)quinolin- 4-yl)benzo[d]thiazol-5- amine |
| 142 | | 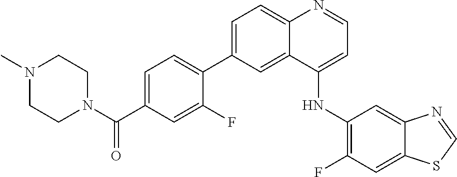 [SMILES: CN1CCN(C(=O)c2ccc(-c3ccc4nccc(Nc5cc6ncsc6cc5F)c4c3)c(F)c2)CC1] | (3-fluoro-4-(4-((6- fluorobenzo[d]thiazol-5- yl)amino)quinolin-6- yl)phenyl)(4- methylpiperazin-1- yl)methanone |
| 143 | | 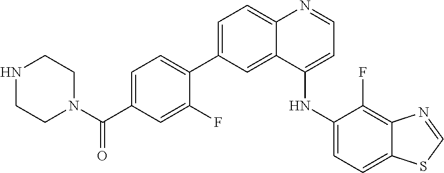 [SMILES: O=C(c1ccc(-c2ccc3nccc(Nc4ccc5scnc5c4F)c3c2)c(F)c1)N1CCNCC1] | (3-fluoro-4-(4-((4- fluorobenzo[d]thiazol-5- yl)amino)quinolin-6- yl)phenyl)(piperazin-1- yl)methanone |
| 144 | | 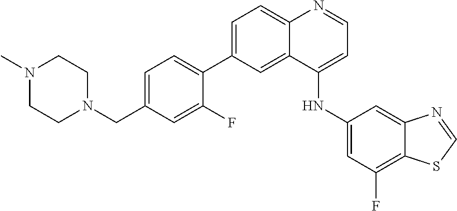 [SMILES: CN1CCN(Cc2ccc(-c3ccc4nccc(Nc5cc(F)c6scnc6c5)c4c3)c(F)c2)CC1] | 7-fluoro-N-(6-(2-fluoro-4- ((4-methylpiperazin-1- yl)methyl)phenyl)quinolin- 4-yl)benzo[d]thiazol-5- amine |
| 145 | | 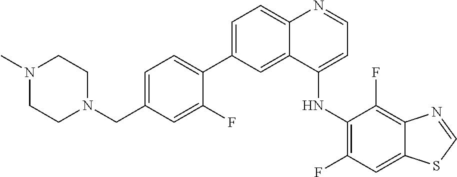 [SMILES: CN1CCN(Cc2ccc(-c3ccc4nccc(Nc5c(F)cc6scnc6c5F)c4c3)c(F)c2)CC1] | 4,6-difluoro-N-(6-(2- fluoro-4-((4- methylpiperazin-1- yl)methyl)phenyl)quinolin- 4-yl)benzo[d]thiazol-5- amine |
| 146 | | 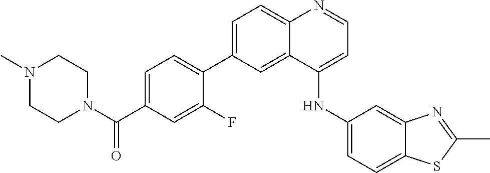 | (3-fluoro-4-(4-((2- methylbenzo[d]thiazol-5- yl)amino)quinolin-6- yl)phenyl)(4- methylpiperazin-1- yl)methanone |
| 147 | | 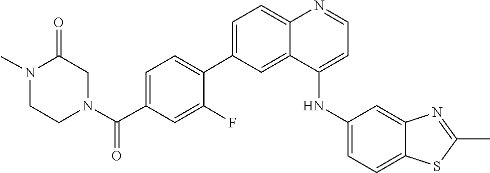 | 4-(3-fluoro-4-(4-((2- methylbenzo[d]thiazol-5- yl)amino)quinolin-6- yl)benzoyl)-1- methylpiperazin-2-one |
| 148 | | 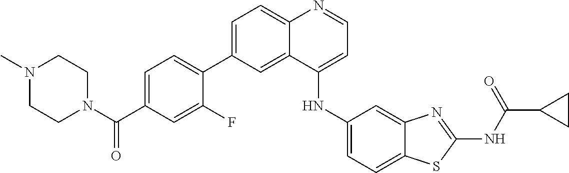 [SMILES: CN1CCN(C(=O)c2ccc(-c3ccc4nccc(Nc5ccc6sc(NC(=O)C7CC7)nc6c5)c4c3)c(F)c2)CC1] | N-(5-((6-(2-fluoro-4-(4- methylpiperazine-1- carbonyl)phenyl)quinolin- 4- yl)amino)benzo[d]thiazol- 2-yl) cyclopropanecarboxamide |
| 149 | | 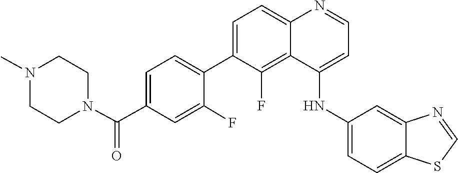 [SMILES: CN1CCN(C(=O)c2ccc(-c3ccc4nccc(Nc5ccc6scnc6c5)c4c3F)c(F)c2)CC1] | (4-(4-(benzo[d]thiazol-5- ylamino)-5-fluoroquinolin- 6-yl)-3-fluorophenyl)(4- methylpiperazin-1- yl)methanone |
| 150 | | 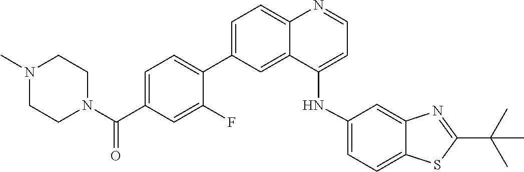 [SMILES: CN1CCN(C(=O)c2ccc(-c3ccc4nccc(Nc5ccc6sc(C(C)(C)C)nc6c5)c4c3)c(F)c2)CC1] | (4-(4-((2-(tert- butyl)benzo[d]thiazol-5- yl)amino)quinolin-6-yl)-3- fluorophenyl)(4- methylpiperazin-1- yl)methanone |
| 151 | | 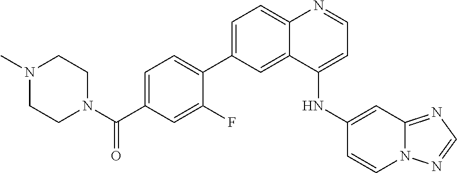 [SMILES: CN1CCN(C(=O)c2ccc(-c3ccc4nccc(Nc5ccn6ncnc6c5)c4c3)c(F)c2)CC1] | (4-(4-([1,2,4]triazolo[1,5- a]pyridin-7- ylamino)quinolin-6-yl)-3- fluorophenyl)(4- methylpiperazin-1- yl)methanone |
| 152 | | 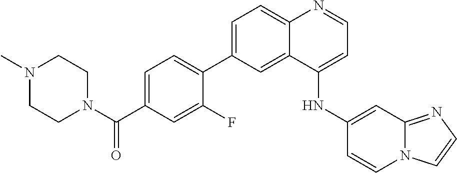 [SMILES: CN1CCN(C(=O)c2ccc(-c3ccc4nccc(Nc5ccn6ccnc6c5)c4c3)c(F)c2)CC1] | (3-fluoro-4-(4- (imidazo[1,2-a]pyridin-7- ylamino)quinolin-6- yl)phenyl)(4- methylpiperazin-1- yl)methanone |
| 153 | | 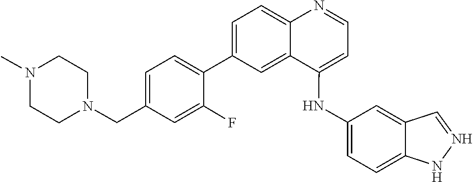 [SMILES: CN1CCN(Cc2ccc(-c3ccc4nccc(Nc5ccc6[nH]ncc6c5)c4c3)c(F)c2)CC1] | N-(1H-2λ⁴-indazol-5-yl)- 6-(2-fluoro-4-((4- methylpiperazin-1- yl)methyl)phenyl)quinolin- 4-amine |
| 154 | | 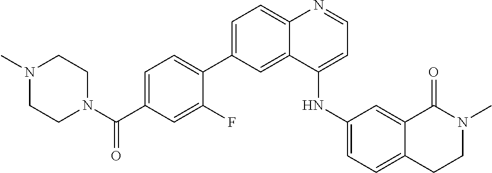 | 7-((6-(2-fluoro-4-(4- methylpiperazine-1- carbonyl)phenyl)quinolin- 4-yl)amino)-2-methyl-3,4- dihydroisoquinolin-1(2H)- one |
| 155 | | 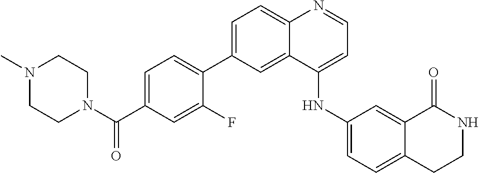 [SMILES: CN1CCN(C(=O)c2ccc(-c3ccc4nccc(Nc5ccc6c(c5)C(=O)NCC6)c4c3)c(F)c2)CC1] | 7-((6-(2-fluoro-4-(4- methylpiperazine-1- carbonyl)phenyl)quinolin- 4-yl)amino)-3,4- dihydroisoquinolin-1(2H)- one |
| 156 | | 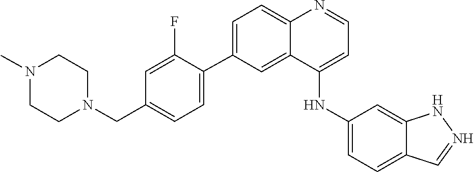 [SMILES: CN1CCN(Cc2ccc(-c3ccc4nccc(Nc5ccc6cn[nH]c6c5)c4c3)c(F)c2)CC1] | N-(1H-2λ⁴-indazol-6-yl)- 6-(2-fluoro-4-((4- methylpiperazin-1- yl)methyl)phenyl)quinolin- 4-amine |
| 157 | | 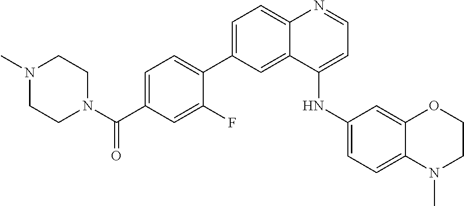 [SMILES: CN1CCN(C(=O)c2ccc(-c3ccc4nccc(Nc5ccc6c(c5)OCCN6C)c4c3)c(F)c2)CC1] | (3-fluoro-4-(4-((4-methyl- 3,4-dihydro-2H- benzo[b][1,4]oxazin-7- yl)amino)quinolin-6- yl)phenyl)(4- methylpiperazin-1- yl)methanone |
| 158 | | 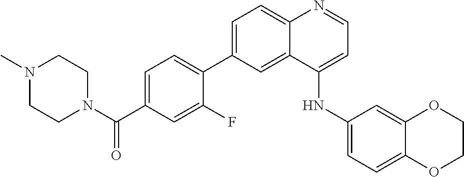 [SMILES: CN1CCN(C(=O)c2ccc(-c3ccc4nccc(Nc5ccc6c(c5)OCCO6)c4c3)c(F)c2)CC1] | (4-(4-((2,3- dihydrobenzo[b][1,4] dioxin-6-yl)amino)quinolin- 6-yl)-3-fluorophenyl)(4- methylpiperazin-1- yl)methanone |
| 159 | | 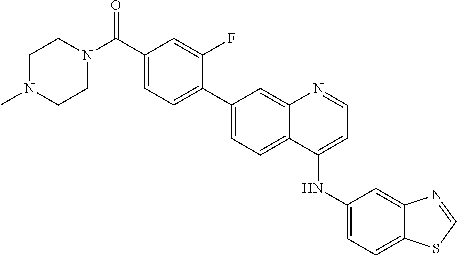 [SMILES: CN1CCN(C(=O)c2ccc(-c3ccc4c(Nc5ccc6scnc6c5)ccnc4c3)c(F)c2)CC1] | (4-(4-(benzo[d]thiazol-5- ylamino)quinolin-7-yl)-3- fluorophenyl)(4- methylpiperazin-1- yl)methanone |
| 160 | | 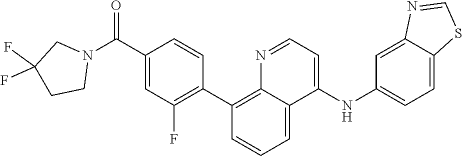 [SMILES: O=C(c1ccc(-c2cccc3c(Nc4ccc5scnc5c4)ccnc23)c(F)c1)N1CCC(F)(F)C1] | (4-(4-(benzo[d]thiazol-5- ylamino)quinolin-8-yl)-3- fluorophenyl)(3,3- difluoropyrrolidin-1- yl)methanone |
| 161 | | 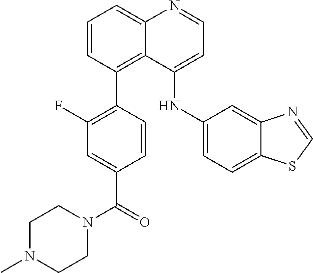 [SMILES: CN1CCN(C(=O)c2ccc(-c3cccc4nccc(Nc5ccc6scnc6c5)c34)c(F)c2)CC1] | (4-(4-(benzo[d]thiazol-5- ylamino)quinolin-5-yl)-3- fluorophenyl)(4- methylpiperazin-1- yl)methanone |
| 162 | | 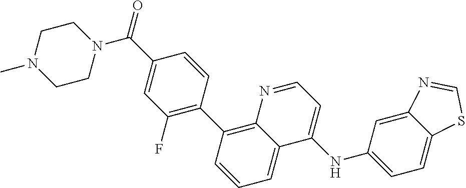 [SMILES: CN1CCN(C(=O)c2ccc(-c3cccc4c(Nc5ccc6scnc6c5)ccnc34)c(F)c2)CC1] | (4-(4-(benzo[d]thiazol-5- ylamino)quinolin-8-yl)-3- fluorophenyl)(4- methylpiperazin-1- yl)methanone |
| 163 | | 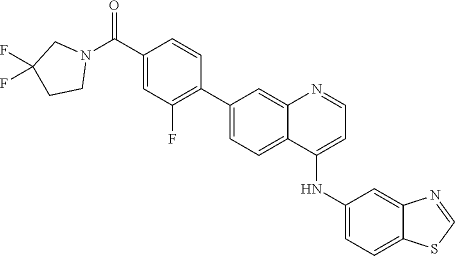 [SMILES: O=C(c1ccc(-c2ccc3c(Nc4ccc5scnc5c4)ccnc3c2)c(F)c1)N1CCC(F)(F)C1] | (4-(4-(benzo[d]thiazol-5- ylamino)quinolin-7-yl)-3- fluorophenyl)(3,3- difluoropyrrolidin-1- yl)methanone |
| 164 | | 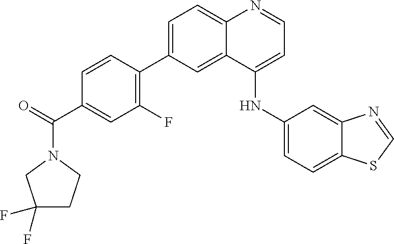 [SMILES: O=C(c1ccc(-c2ccc3nccc(Nc4ccc5scnc5c4)c3c2)c(F)c1)N1CCC(F)(F)C1] | (4-(4-(benzo[d]thiazol-5- ylamino)quinolin-6-yl)-3- fluorophenyl)(3,3- difluoropyrrolidin-1- yl)methanone |
| 177 | | 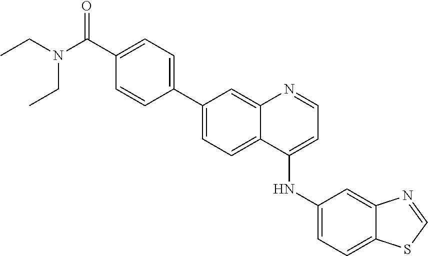 [SMILES: CCN(CC)C(=O)c1ccc(-c2ccc3c(Nc4ccc5scnc5c4)ccnc3c2)cc1] | 4-(4-(benzo[d]thiazol-5- ylamino)quinolin-7-yl)- N,N-diethylbenzamide |
| 178 | | 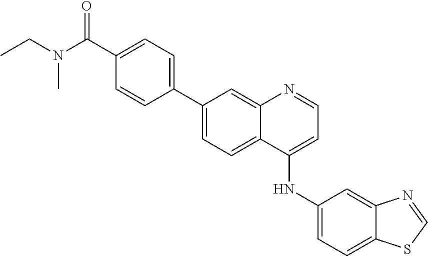 [SMILES: CCN(C)C(=O)c1ccc(-c2ccc3c(Nc4ccc5scnc5c4)ccnc3c2)cc1] | 4-(4-(benzo[d]thiazol-5- ylamino)quinolin-7-yl)-N- ethyl-N-methylbenzamide |
| 179 | | 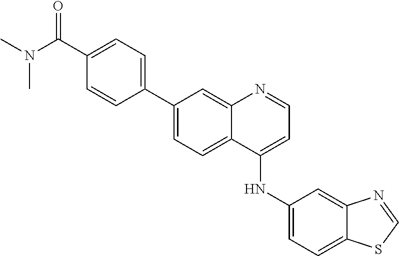 [SMILES: CN(C)C(=O)c1ccc(-c2ccc3c(Nc4ccc5scnc5c4)ccnc3c2)cc1] | 4-(4-(benzo[d]thiazol-5- ylamino)quinolin-7-yl)- N,N-dimethylbenzamide |
| 180 | | 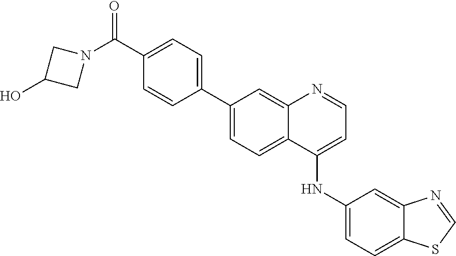 [SMILES: O=C(c1ccc(-c2ccc3c(Nc4ccc5scnc5c4)ccnc3c2)cc1)N1CC(O)C1] | (4-(4-(benzo[d]thiazol-5- ylamino)quinolin-7- yl)phenyl)(3- hydroxyazetidin-1- yl)methanone |
| 181 | | 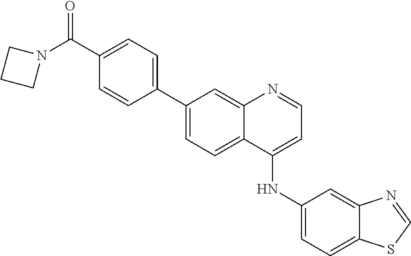 [SMILES: O=C(c1ccc(-c2ccc3c(Nc4ccc5scnc5c4)ccnc3c2)cc1)N1CCC1] | azetidin-1-yl(4-(4- (benzo[d]thiazol-5- ylamino)quinolin-7- yl)phenyl)methanone |
| 182 | | 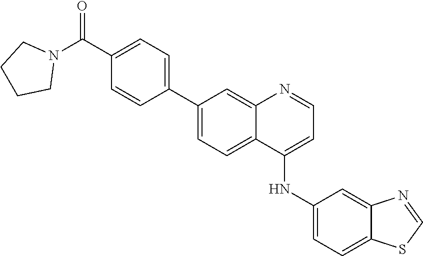 [SMILES: O=C(c1ccc(-c2ccc3c(Nc4ccc5scnc5c4)ccnc3c2)cc1)N1CCCC1] | (4-(4-(benzo[d]thiazol-5- ylamino)quinolin-7- yl)phenyl)(pyrrolidin-1- yl)methanone |
| 183 | | 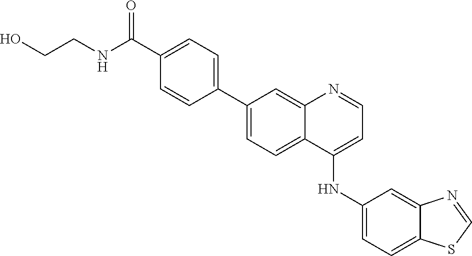 [SMILES: O=C(NCCO)c1ccc(-c2ccc3c(Nc4ccc5scnc5c4)ccnc3c2)cc1] | 4-(4-(benzo[d]thiazol-5- ylamino)quinolin-7-yl)-N- (2- hydroxyethyl)benzamide |
| 184 | | 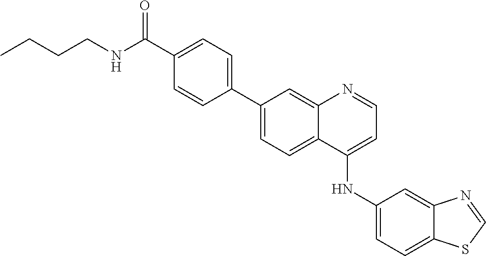 [SMILES: CCCCNC(=O)c1ccc(-c2ccc3c(Nc4ccc5scnc5c4)ccnc3c2)cc1] | 4-(4-(benzo[d]thiazol-5- ylamino)quinolin-7-yl)-N- butylbenzamide |
| 185 | | 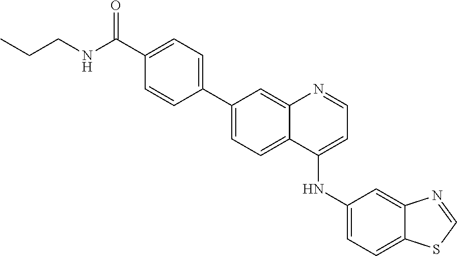 [SMILES: CCCNC(=O)c1ccc(-c2ccc3c(Nc4ccc5scnc5c4)ccnc3c2)cc1] | 4-(4-(benzo[d]thiazol-5- ylamino)quinolin-7-yl)-N- propylbenzamide |
| 186 | | 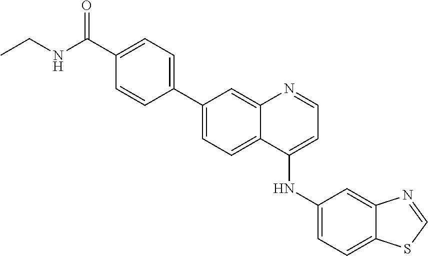 [SMILES: CCNC(=O)c1ccc(-c2ccc3c(Nc4ccc5scnc5c4)ccnc3c2)cc1] | 4-(4-(benzo[d]thiazol-5- ylamino)quinolin-7-yl)-N- ethylbenzamide |
| 187 | | 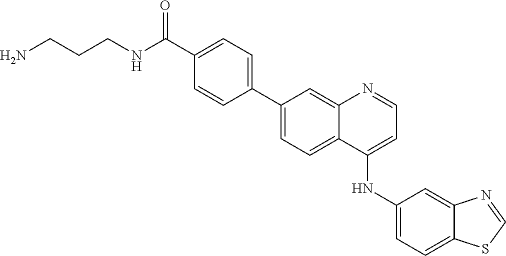 [SMILES: NCCCNC(=O)c1ccc(-c2ccc3c(Nc4ccc5scnc5c4)ccnc3c2)cc1] | N-(3-aminopropyl)-4-(4- (benzo[d]thiazol-5- ylamino)quinolin-7- yl)benzamide |
| 188 | | 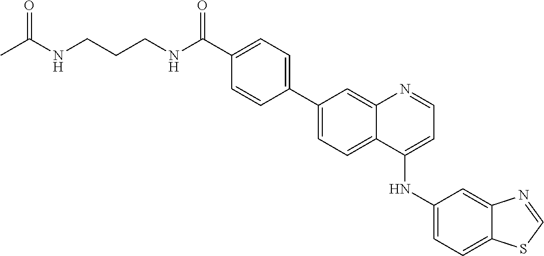 [SMILES: CC(=O)NCCCNC(=O)c1ccc(-c2ccc3c(Nc4ccc5scnc5c4)ccnc3c2)cc1] | N-(3-acetamidopropyl)-4- (4-(benzo[d]thiazol-5- ylamino)quinolin-7- yl)benzamide |
| 189 | | 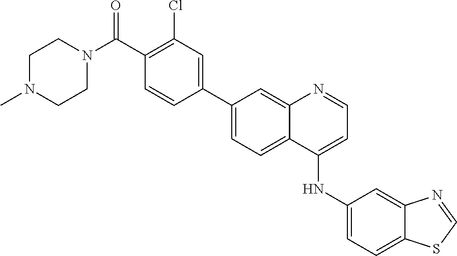 [SMILES: CN1CCN(C(=O)c2ccc(-c3ccc4c(Nc5ccc6scnc6c5)ccnc4c3)cc2Cl)CC1] | (4-(4-(benzo[d]thiazol-5- ylamino)quinolin-7-yl)-2- chlorophenyl)(4- methylpiperazin-1- yl)methanone |
| 190 | | 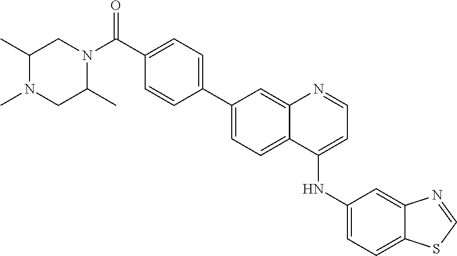 [SMILES: CC1CN(C(=O)c2ccc(-c3ccc4c(Nc5ccc6scnc6c5)ccnc4c3)cc2)C(C)CN1C] | (4-(4-(benzo[d]thiazol-5- ylamino)quinolin-7- yl)phenyl)(2,4,5- trimethylpiperazin-1- yl)methanone |
| 191 | | 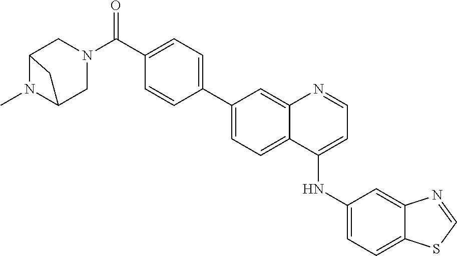 [SMILES: CN1C2CC1CN(C(=O)c1ccc(-c3ccc4c(Nc5ccc6scnc6c5)ccnc4c3)cc1)C2] | (4-(4-(benzo[d]thiazol-5- ylamino)quinolin-7- yl)phenyl)(6-methyl-3,6- diazabicyclo[3.1.1]heptan- 3-yl)methanone |
| 192 | | 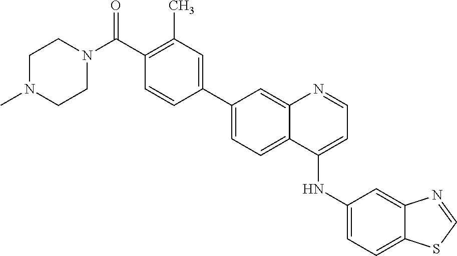 [SMILES: Cc1cc(-c2ccc3c(Nc4ccc5scnc5c4)ccnc3c2)ccc1C(=O)N1CCN(C)CC1] | (4-(4-(benzo[d]thiazol-5- ylamino)quinolin-7-yl)-2- methylphenyl)(4- methylpiperazin-1- yl)methanone |
| 193 | | 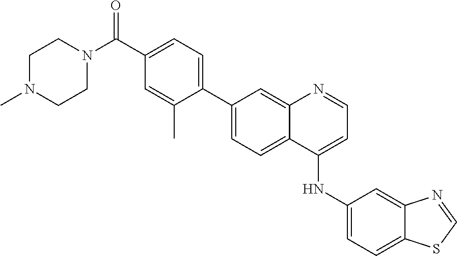 [SMILES: Cc1cc(C(=O)N2CCN(C)CC2)ccc1-c1ccc2c(Nc3ccc4scnc4c3)ccnc2c1] | (4-(4-(benzo[d]thiazol-5- ylamino)quinolin-7-yl)-3- methylphenyl)(4- methylpiperazin-1- yl)methanone |
| 194 | | 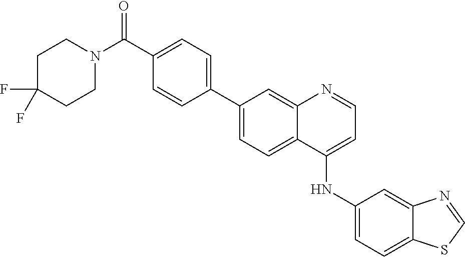 [SMILES: O=C(c1ccc(-c2ccc3c(Nc4ccc5scnc5c4)ccnc3c2)cc1)N1CCC(F)(F)CC1] | (4-(4-(benzo[d]thiazol-5- ylamino)quinolin-7- yl)phenyl)(4,4- difluoropiperidin-1- yl)methanone |
| 195 | | 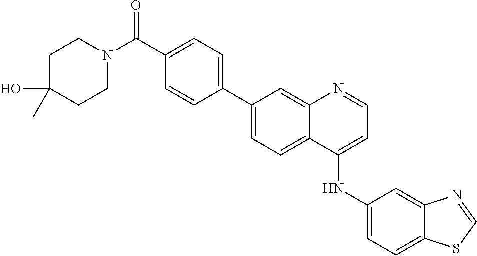 [SMILES: CC1(O)CCN(C(=O)c2ccc(-c3ccc4c(Nc5ccc6scnc6c5)ccnc4c3)cc2)CC1] | (4-(4-(benzo[d]thiazol-5- ylamino)quinolin-7- yl)phenyl)(4-hydroxy-4- methylpiperidin-1- yl)methanone |
| 196 | | 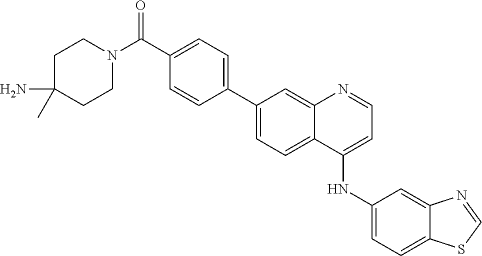 [SMILES: CC1(N)CCN(C(=O)c2ccc(-c3ccc4c(Nc5ccc6scnc6c5)ccnc4c3)cc2)CC1] | (4-amino-4- methylpiperidin-1-yl)(4- (4-(benzo[d]thiazol-5- ylamino)quinolin-7- yl)phenyl)methanone |
| 197 | | 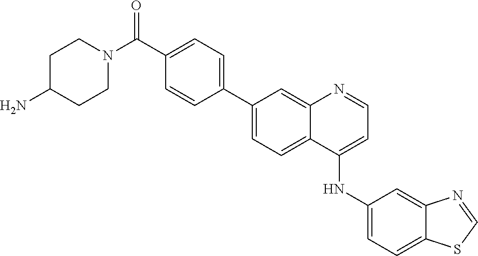 [SMILES: NC1CCN(C(=O)c2ccc(-c3ccc4c(Nc5ccc6scnc6c5)ccnc4c3)cc2)CC1] | (4-aminopiperidin-1-yl)(4- (4-(benzo[d]thiazol-5- ylamino)quinolin-7- yl)phenyl)methanone |
| 198 | | 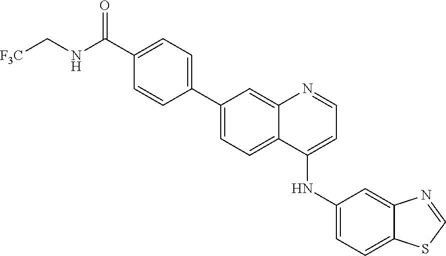 [SMILES: O=C(NCC(F)(F)F)c1ccc(-c2ccc3c(Nc4ccc5scnc5c4)ccnc3c2)cc1] | 4-(4-(benzo[d]thiazol-5- ylamino)quinolin-7-yl)-N- (2,2,2- trifluoroethyl)benzamide |
| 199 | | 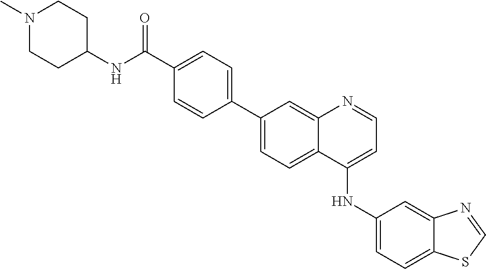 [SMILES: CN1CCC(NC(=O)c2ccc(-c3ccc4c(Nc5ccc6scnc6c5)ccnc4c3)cc2)CC1] | 4-(4-(benzo[d]thiazol-5- ylamino)quinolin-7-yl)-N- (1-methylpiperidin-4- yl)benzamide |
| 200 | | 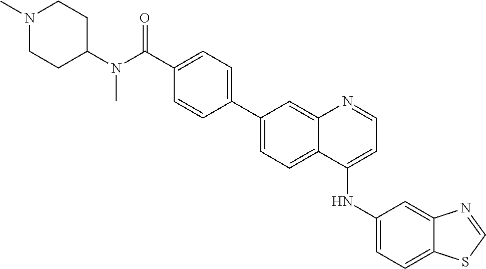 [SMILES: CN1CCC(N(C)C(=O)c2ccc(-c3ccc4c(Nc5ccc6scnc6c5)ccnc4c3)cc2)CC1] | 4-(4-(benzo[d]thiazol-5- ylamino)quinolin-7-yl)-N- methyl-N-(1- methylpiperidin-4- yl)benzamide |
| 201 | | 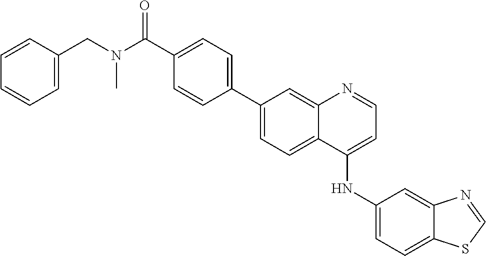 [SMILES: CN(Cc1ccccc1)C(=O)c1ccc(-c2ccc3c(Nc4ccc5scnc5c4)ccnc3c2)cc1] | 4-(4-(benzo[d]thiazol-5- ylamino)quinolin-7-yl)-N- benzyl-N- methylbenzamide |
| 202 | | 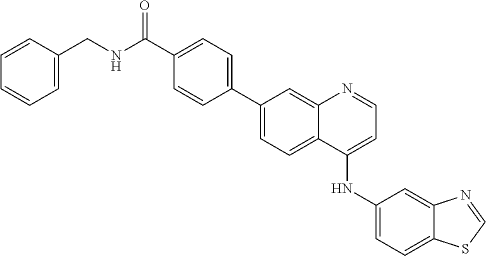 [SMILES: O=C(NCc1ccccc1)c1ccc(-c2ccc3c(Nc4ccc5scnc5c4)ccnc3c2)cc1] | 4-(4-(benzo[d]thiazol-5- ylamino)quinolin-7-yl)-N- benzylbenzamide |
| 203 | | 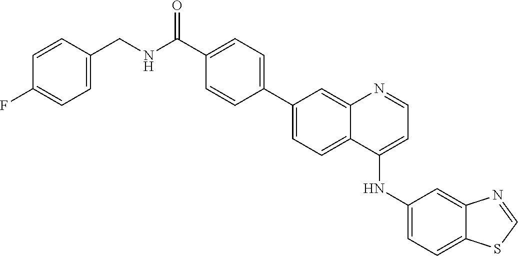 [SMILES: O=C(NCc1ccc(F)cc1)c1ccc(-c2ccc3c(Nc4ccc5scnc5c4)ccnc3c2)cc1] | 4-(4-(benzo[d]thiazol-5- ylamino)quinolin-7-yl)-N- (4- fluorobenzyl)benzamide |
| 210 | | 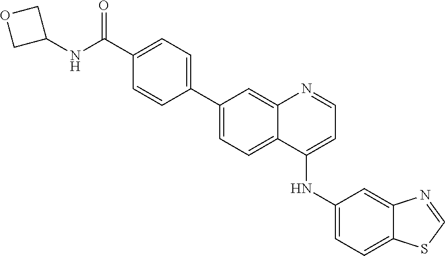 [SMILES: O=C(NC1COC1)c1ccc(-c2ccc3c(Nc4ccc5scnc5c4)ccnc3c2)cc1] | 4-(4-(benzo[d]thiazol-5- ylamino)quinolin-7-yl)-N- (oxetan-3-yl)benzamide |
| 211 | | 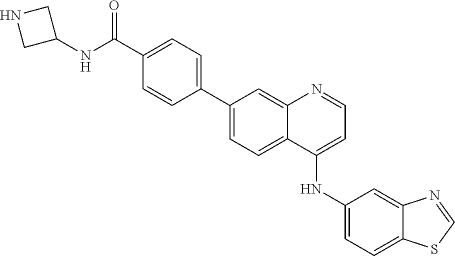 [SMILES: O=C(NC1CNC1)c1ccc(-c2ccc3c(Nc4ccc5scnc5c4)ccnc3c2)cc1] | N-(azetidin-3-yl)-4-(4- (benzo[d]thiazol-5- ylamino)quinolin-7- yl)benzamide |
| 212 | | 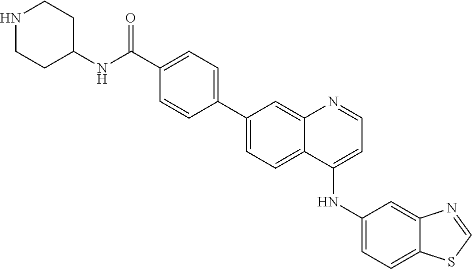 [SMILES: O=C(NC1CCNCC1)c1ccc(-c2ccc3c(Nc4ccc5scnc5c4)ccnc3c2)cc1] | 4-(4-(benzo[d]thiazol-5- ylamino)quinolin-7-yl)-N- (piperidin-4-yl)benzamide |
| 213 | | 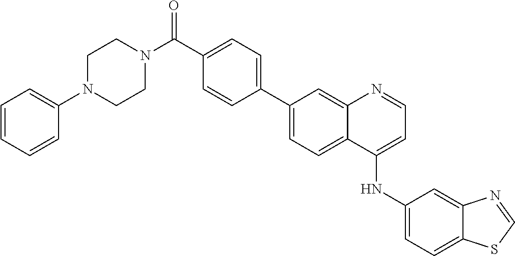 [SMILES: O=C(c1ccc(-c2ccc3c(Nc4ccc5scnc5c4)ccnc3c2)cc1)N1CCN(c2ccccc2)CC1] | (4-(4-(benzo[d]thiazol-5- ylamino)quinolin-7- yl)phenyl)(4- phenylpiperazin-1- yl)methanone |
| 214 | | 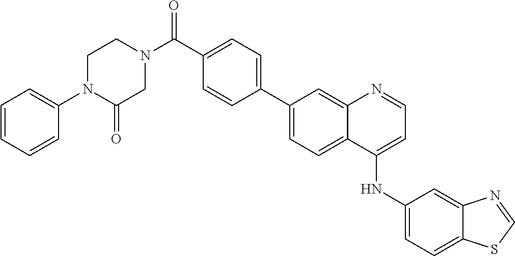 [SMILES: O=C(c1ccc(-c2ccc3c(Nc4ccc5scnc5c4)ccnc3c2)cc1)N1CCN(c2ccccc2)C(=O)C1] | 4-(4-(4-(benzo[d]thiazol- 5-ylamino)quinolin-7- yl)benzoyl)-1- phenylpiperazin-2-one |
| 215 | | 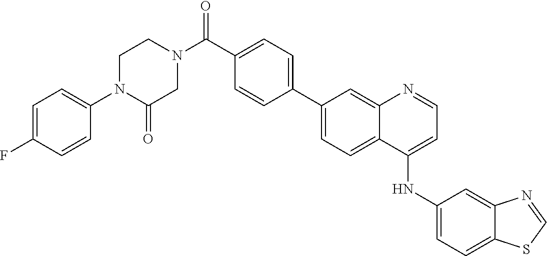 [SMILES: O=C(c1ccc(-c2ccc3c(Nc4ccc5scnc5c4)ccnc3c2)cc1)N1CCN(c2ccc(F)cc2)C(=O)C1] | 4-(4-(4-(benzo[d]thiazol- 5-ylamino)quinolin-7- yl)benzoyl)-1-(4- fluorophenyl)piperazin-2- one |
| 216 | | 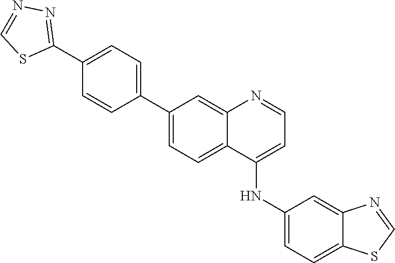 [SMILES: c1cc(Nc2ccc3scnc3c2)c2ccc(-c3ccc(-c4nncs4)cc3)cc2n1] | N-(7-(4-(1,3,4-thiadiazol- 2-yl)phenyl)quinolin-4- yl)benzo[d]thiazol-5- amine |
| 217 | | 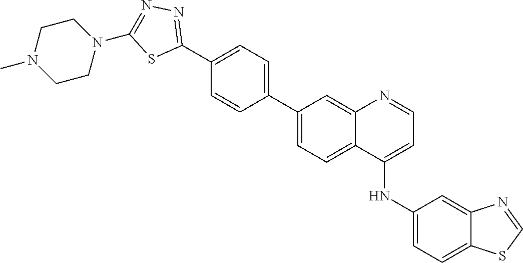 [SMILES: CN1CCN(c2nnc(-c3ccc(-c4ccc5c(Nc6ccc7scnc7c6)ccnc5c4)cc3)s2)CC1] | N-(7-(4-(5-(4- methylpiperazin-1-yl)- 1,3,4-thiadiazol-2- yl)phenyl)quinolin-4- yl)benzo[d]thiazol-5- amine |
| 218 | | 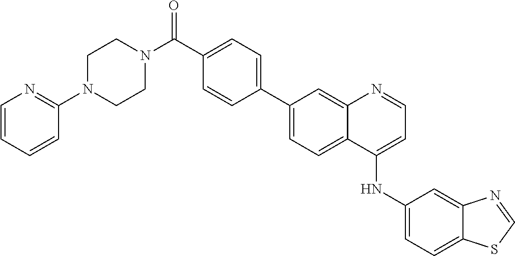 [SMILES: O=C(c1ccc(-c2ccc3c(Nc4ccc5scnc5c4)ccnc3c2)cc1)N1CCN(c2ccccn2)CC1] | (4-(4-(benzo[d]thiazol-5- ylamino)quinolin-7- yl)phenyl)(4-(pyridin-2- yl)piperazin-1- yl)methanone |
| 219 | | 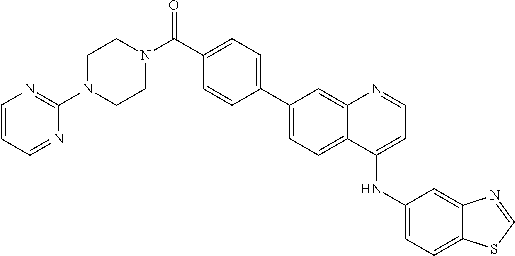 [SMILES: O=C(c1ccc(-c2ccc3c(Nc4ccc5scnc5c4)ccnc3c2)cc1)N1CCN(c2ncccn2)CC1] | (4-(4-(benzo[d]thiazol-5- ylamino)quinolin-7- yl)phenyl)(4-(pyrimidin-2- yl)piperazin-1- yl)methanone |
| 220 | | 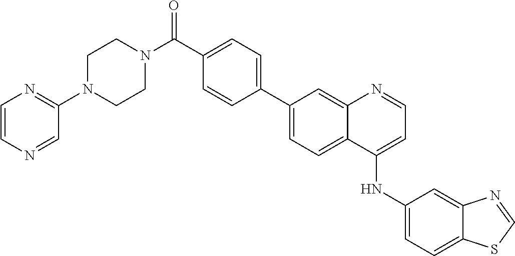 [SMILES: O=C(c1ccc(-c2ccc3c(Nc4ccc5scnc5c4)ccnc3c2)cc1)N1CCN(c2cnccn2)CC1] | (4-(4-(benzo[d]thiazol-5- ylamino)quinolin-7- yl)phenyl)(4-(pyrazin-2- yl)piperazin-1- yl)methanone |
| 221 | | 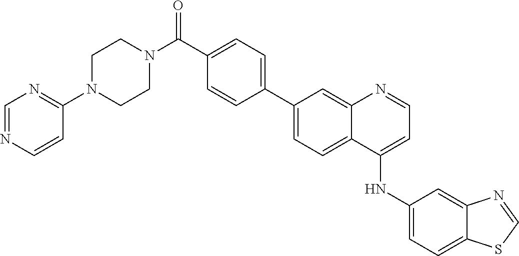 [SMILES: O=C(c1ccc(-c2ccc3c(Nc4ccc5scnc5c4)ccnc3c2)cc1)N1CCN(c2ccncn2)CC1] | (4-(4-(benzo[d]thiazol-5- ylamino)quinolin-7- yl)phenyl)(4-(pyrimidin-4- yl)piperazin-1- yl)methanone |
| 222 | | 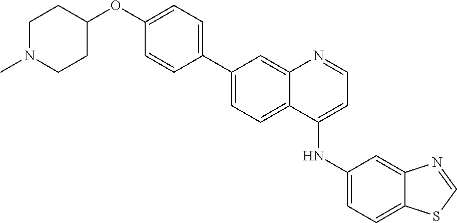 [SMILES: CN1CCC(Oc2ccc(-c3ccc4c(Nc5ccc6scnc6c5)ccnc4c3)cc2)CC1] | N-(7-(4-((1- methylpiperidin-4- yl)oxy)phenyl)quinolin-4- yl)benzo[d]thiazol-5- amine |
| 223 | | 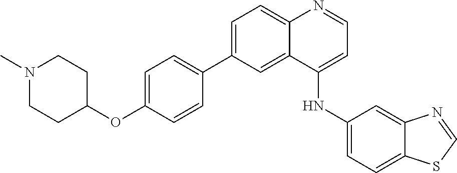 [SMILES: CN1CCC(Oc2ccc(-c3ccc4nccc(Nc5ccc6scnc6c5)c4c3)cc2)CC1] | N-(6-(4-((1- methylpiperidin-4- yl)oxy)phenyl)quinolin-4- yl)benzo[d]thiazol-5- amine |
| 224 (also referred to as Com- pound 75) | | 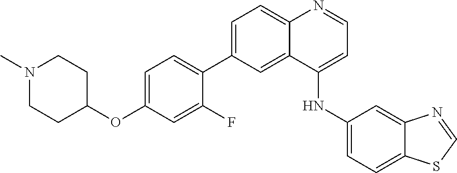 [SMILES: CN1CCC(Oc2ccc(-c3ccc4nccc(Nc5ccc6scnc6c5)c4c3)c(F)c2)CC1] | N-(6-(2-fluoro-4-((1- methylpiperidin-4- yl)oxy)phenyl)quinolin-4- yl)benzo[d]thiazol-5- amine |
| 225 | | 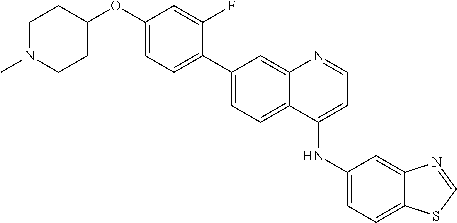 [SMILES: CN1CCC(Oc2ccc(-c3ccc4c(Nc5ccc6scnc6c5)ccnc4c3)c(F)c2)CC1] | N-(7-(2-fluoro-4-((1- methylpiperidin-4- yl)oxy)phenyl)quinolin-4- yl)benzo[d]thiazol-5- amine |
| 226 | | 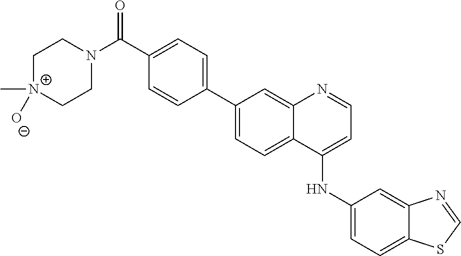 [SMILES: C[N+]1([O-])CCN(C(=O)c2ccc(-c3ccc4c(Nc5ccc6scnc6c5)ccnc4c3)cc2)CC1] | 4-(4-(4-(benzo[d]thiazol- 5-ylamino)quinolin-7- yl)benzoyl)-1- methylpiperazine 1-oxide |
| 227 | | 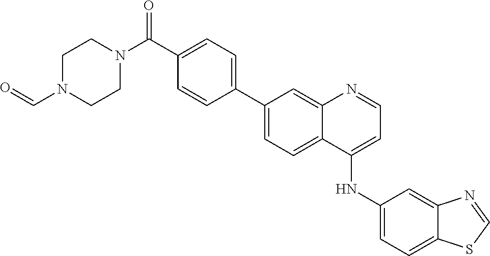 [SMILES: O=CN1CCN(C(=O)c2ccc(-c3ccc4c(Nc5ccc6scnc6c5)ccnc4c3)cc2)CC1] | 4-(4-(4-(benzo[d]thiazol- 5-ylamino)quinolin-7- yl)benzoyl)piperazine-1- carbaldehyde |
| 228 | | 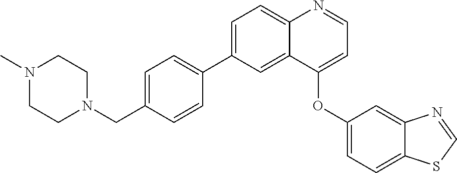 [SMILES: CN1CCN(Cc2ccc(-c3ccc4nccc(Oc5ccc6scnc6c5)c4c3)cc2)CC1] | 5-((6-(4-((4- methylpiperazin-1- yl)methyl)phenyl)quinolin- 4-yl)oxy)benzo[d]thiazole |
| 229 | | 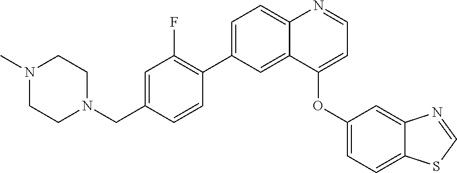 [SMILES: CN1CCN(Cc2ccc(-c3ccc4nccc(Oc5ccc6scnc6c5)c4c3)c(F)c2)CC1] | 5-((6-(2-fluoro-4-((4- methylpiperazin-1- yl)methyl)phenyl)quinolin- 4-yl)oxy)benzo[d]thiazole |
| 230 | | 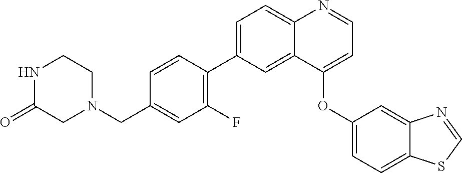 [SMILES: O=C1CN(Cc2ccc(-c3ccc4nccc(Oc5ccc6scnc6c5)c4c3)c(F)c2)CCN1] | 4-(4-(4-(benzo[d]thiazol- 5-yloxy)quinolin-6-yl)-3- fluorobenzyl)piperazin-2- one |
| 231 (also referred to as Com- pound 373) | | 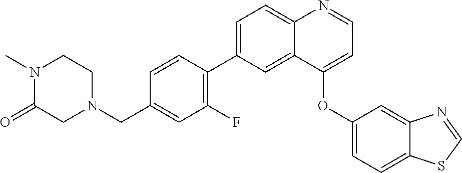 [SMILES: CN1CCN(Cc2ccc(-c3ccc4nccc(Oc5ccc6scnc6c5)c4c3)c(F)c2)CC1=O] | 4-(4-(4-(benzo[d]thiazol- 5-yloxy)quinolin-6-yl)-3- fluorobenzyl)-1- methylpiperazin-2-one |
| 232 | | 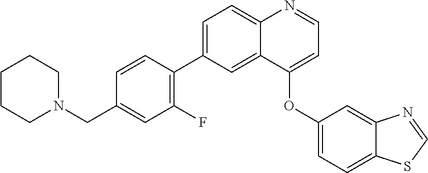 [SMILES: Fc1cc(CN2CCCCC2)ccc1-c1ccc2nccc(Oc3ccc4scnc4c3)c2c1] | 5-((6-(2-fluoro-4- (piperidin-1- ylmethyl)phenyl)quinolin- 4-yl)oxy)benzo[d]thiazole |
| 233 | | 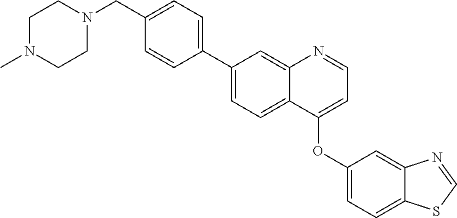 [SMILES: CN1CCN(Cc2ccc(-c3ccc4c(Oc5ccc6scnc6c5)ccnc4c3)cc2)CC1] | 5-((7-(4-((4- methylpiperazin-1- yl)methyl)phenyl)quinolin- 4-yl)oxy)benzo[d]thiazole |
| 234 | | 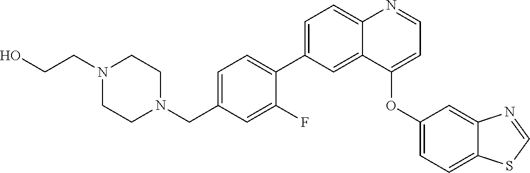 [SMILES: OCCN1CCN(Cc2ccc(-c3ccc4nccc(Oc5ccc6scnc6c5)c4c3)c(F)c2)CC1] | 2-(4-(4-(4- (benzo[d]thiazol-5- yloxy)quinolin-6-yl)-3- fluorobenzyl)piperazin-1- yl)ethan-1-ol |
| 235 | | 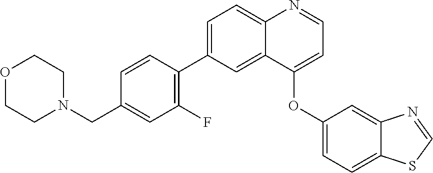 [SMILES: Fc1cc(CN2CCOCC2)ccc1-c1ccc2nccc(Oc3ccc4scnc4c3)c2c1] | 4-(4-(4-(benzo[d]thiazol- 5-yloxy)quinolin-6-yl)-3- fluorobenzyl)morpholine |
| 236 | | 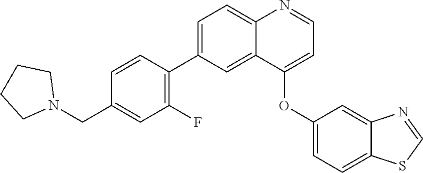 [SMILES: Fc1cc(CN2CCCC2)ccc1-c1ccc2nccc(Oc3ccc4scnc4c3)c2c1] | 5-((6-(2-fluoro-4- (pyrrolidin-1- ylmethyl)phenyl)quinolin- 4-yl)oxy)benzo[d]thiazole |
| 237 | | 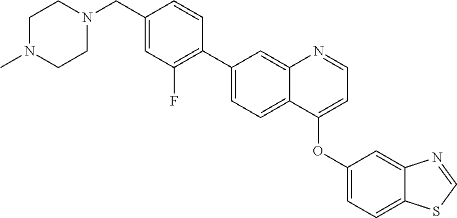 [SMILES: CN1CCN(Cc2ccc(-c3ccc4c(Oc5ccc6scnc6c5)ccnc4c3)c(F)c2)CC1] | 5-((7-(2-fluoro-4-((4- methylpiperazin-1- yl)methyl)phenyl)quinolin- 4-yl)oxy)benzo[d]thiazole |
| 238 | | 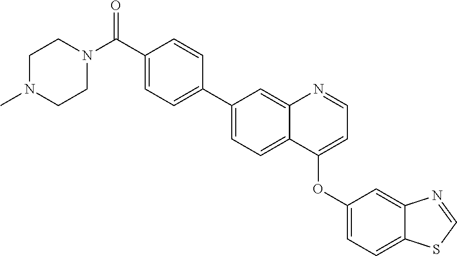 [SMILES: CN1CCN(C(=O)c2ccc(-c3ccc4c(Oc5ccc6scnc6c5)ccnc4c3)cc2)CC1] | (4-(4-(benzo[d]thiazol-5- yloxy)quinolin-7- yl)phenyl)(4- methylpiperazin-1- yl)methanone |
| 239 | 56 | 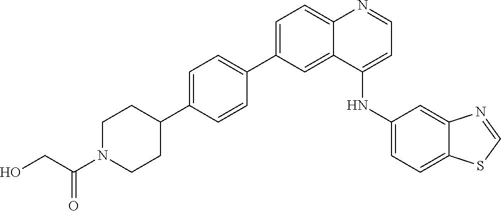 [SMILES: O=C(CO)N1CCC(c2ccc(-c3ccc4nccc(Nc5ccc6scnc6c5)c4c3)cc2)CC1] | 1-(4-(4-(4- (benzo[d]thiazol-5- ylamino)quinolin-6- yl)phenyl)piperidin-1-yl)- 2-hydroxyethan-1-one |
| 240 | 57 | 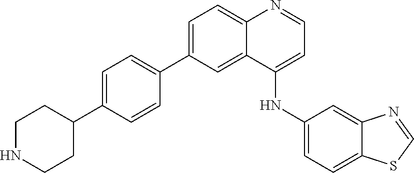 [SMILES: c1cc(Nc2ccc3scnc3c2)c2cc(-c3ccc(C4CCNCC4)cc3)ccc2n1] | N-(6-(4-(piperidin-4- yl)phenyl)quinolin-4- yl)benzo[d]thiazol-5-amine |
| 241 (also referred to as Com- pound 100) | 58 | 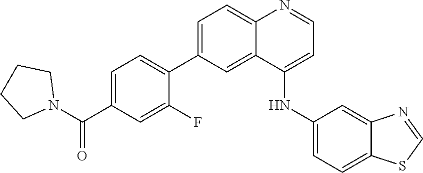 [SMILES: O=C(c1ccc(-c2ccc3nccc(Nc4ccc5scnc5c4)c3c2)c(F)c1)N1CCCC1] | (4-(4-(benzo[d]thiazol-5- ylamino)quinolin-6-yl)-3- fluorophenyl)(pyrrolidin-1- yl)methanone |
| 242 | 59 | 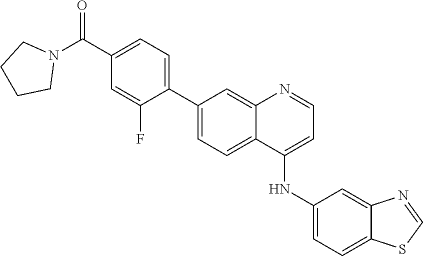 [SMILES: O=C(c1ccc(-c2ccc3c(Nc4ccc5scnc5c4)ccnc3c2)c(F)c1)N1CCCC1] | (4-(4-(benzo[d]thiazol-5- ylamino)quinolin-7-yl)-3- fluorophenyl)(pyrrolidin-1- yl)methanone |
| 243 (also referred to as Com- pound 104) | 60 | 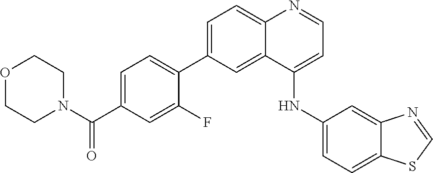 [SMILES: O=C(c1ccc(-c2ccc3nccc(Nc4ccc5scnc5c4)c3c2)c(F)c1)N1CCOCC1] | (4-(4-(benzo[d]thiazol-5- ylamino)quinolin-6-yl)-3- fluorophenyl)(morpholino) methanone |
| 244 | 61 | 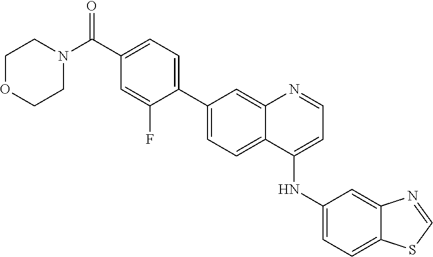 [SMILES: O=C(c1ccc(-c2ccc3c(Nc4ccc5scnc5c4)ccnc3c2)c(F)c1)N1CCOCC1] | (4-(4-(benzo[d]thiazol-5- ylamino)quinolin-7-yl)-3- fluorophenyl)(morpholino) methanone |
| 245 (also referred to as Com- pound 342) | 62 | 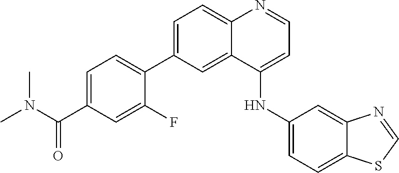 [SMILES: CN(C)C(=O)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N,N- dimethylbenzamide |
| 246 | 63 | 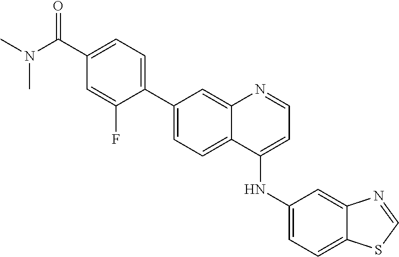 [SMILES: CN(C)C(=O)c1ccc(-c2ccc3c(Nc4ccc5scnc5c4)ccnc3c2)c(F)c1] | 4-(4-(benzo[d]thiazol-5- ylamino)quinolin-7-yl)-3- fluoro-N,N- dimethylbenzamide |
| 247 | 64 | 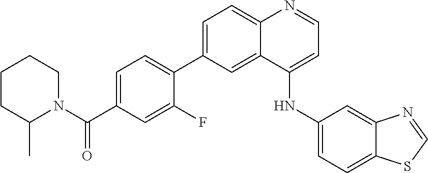 [SMILES: CC1CCCCN1C(=O)c1ccc(-c2ccc3nccc(Nc4ccc5scnc5c4)c3c2)c(F)c1] | (4-(4-(benzo[d]thiazol-5- ylamino)quinolin-6-yl)-3- fluorophenyl)(2- methylpiperidin-1- yl)methanone |
| 248 | 65 | 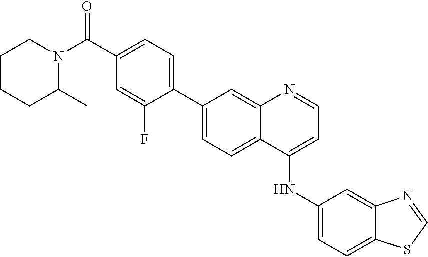 [SMILES: CC1CCCCN1C(=O)c1ccc(-c2ccc3c(Nc4ccc5scnc5c4)ccnc3c2)c(F)c1] | (4-(4-(benzo[d]thiazol-5- ylamino)quinolin-7-yl)-3- fluorophenyl)(2- methylpiperidin-1- yl)methanone |
| 249 | 75 | 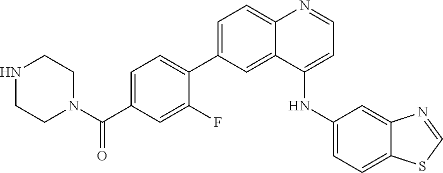 [SMILES: O=C(c1ccc(-c2ccc3nccc(Nc4ccc5scnc5c4)c3c2)c(F)c1)N1CCNCC1] | (4-(4-(benzo[d]thiazol-5- ylamino)quinolin-6-yl)-3- fluorophenyl)(piperazin-1- yl)methanone |
| 250 | 76 | 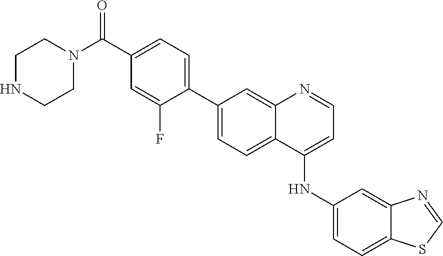 [SMILES: O=C(c1ccc(-c2ccc3c(Nc4ccc5scnc5c4)ccnc3c2)c(F)c1)N1CCNCC1] | (4-(4-(benzo[d]thiazol-5- ylamino)quinolin-7-yl)-3- fluorophenyl)(piperazin-1- yl)methanone |
| 251 | 73 | 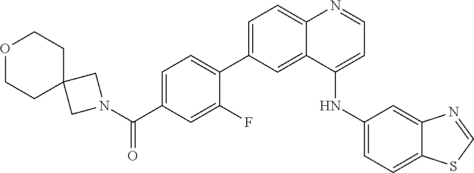 [SMILES: O=C(c1ccc(-c2ccc3nccc(Nc4ccc5scnc5c4)c3c2)c(F)c1)N1CC2(CCOCC2)C1] | (4-(4-(benzo[d]thiazol-5- ylamino)quinolin-6-yl)-3- fluorophenyl)(7-oxa-2- azaspiro[3.5]nonan-2- yl)methanone |
| 252 | 74 | 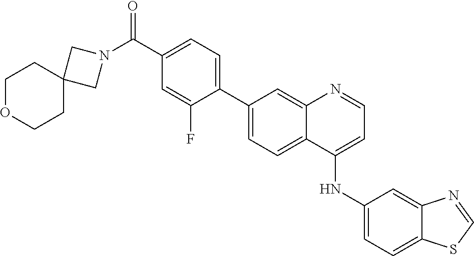 [SMILES: O=C(c1ccc(-c2ccc3c(Nc4ccc5scnc5c4)ccnc3c2)c(F)c1)N1CC2(CCOCC2)C1] | (4-(4-(benzo[d]thiazol-5- ylamino)quinolin-7-yl)-3- fluorophenyl)(7-oxa-2- azaspiro[3.5]nonan-2- yl)methanone |
| 253 | 66 | 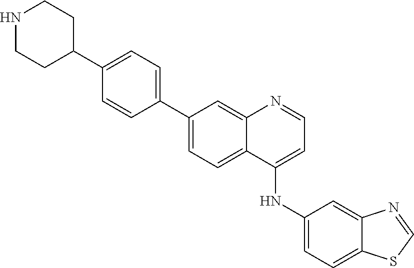 [SMILES: c1cc(Nc2ccc3scnc3c2)c2ccc(-c3ccc(C4CCNCC4)cc3)cc2n1] | N-(7-(4-(piperidin-4- yl)phenyl)quinolin-4- yl)benzo[d]thiazol-5-amine |
| 254 (also referred to as Com- pound 74 and Com- pound 295) | 95 | 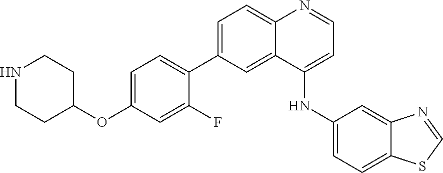 [SMILES: Fc1cc(OC2CCNCC2)ccc1-c1ccc2nccc(Nc3ccc4scnc4c3)c2c1] | N-(6-(2-fluoro-4- (piperidin-4- yloxy)phenyl)quinolin-4- yl)benzo[d]thiazol-5-amine |
| 255 (also referred to as Com- pound 385) | 78 | 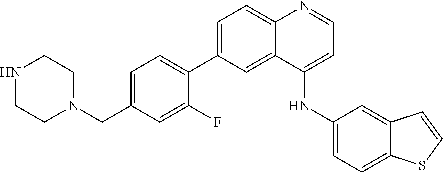 [SMILES: Fc1cc(CN2CCNCC2)ccc1-c1ccc2nccc(Nc3ccc4sccc4c3)c2c1] | N-(benzo[b]thiophen-5-yl)- 6-(2-fluoro-4-(piperazin-1- ylmethyl)phenyl)quinolin- 4-amine |
| 256 | 72 | 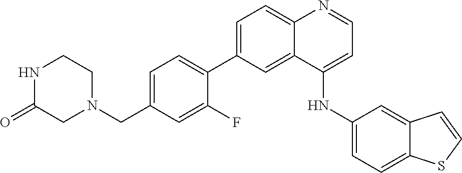 [SMILES: O=C1CN(Cc2ccc(-c3ccc4nccc(Nc5ccc6sccc6c5)c4c3)c(F)c2)CCN1] | 4-(4-(4-(benzo[b]thiophen- 5-ylamino)quinolin-6-yl)- 3-fluorobenzyl)piperazin- 2-one |
| 257 | | 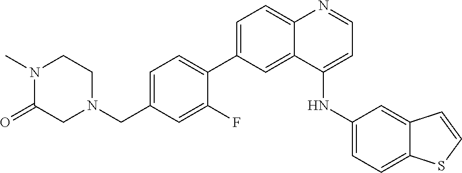 [SMILES: CN1CCN(Cc2ccc(-c3ccc4nccc(Nc5ccc6sccc6c5)c4c3)c(F)c2)CC1=O] | 4-(4-(4-(benzo[b]thiophen- 5-ylamino)quinolin-6-yl)- 3-fluorobenzyl)-1- methylpiperazin-2-one |
| 258 | | 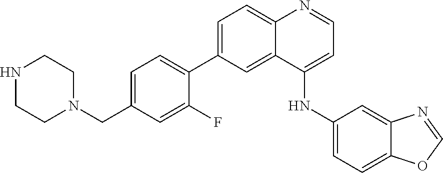 [SMILES: Fc1cc(CN2CCNCC2)ccc1-c1ccc2nccc(Nc3ccc4ocnc4c3)c2c1] | N-(6-(2-fluoro-4- (piperazin-1- ylmethyl)phenyl)quinolin- 4-yl)benzo[d]oxazol-5- amine |
| 259 | | 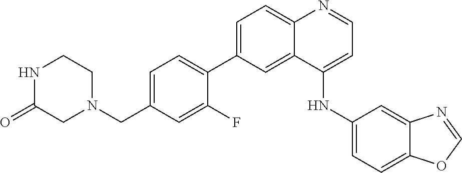 [SMILES: O=C1CN(Cc2ccc(-c3ccc4nccc(Nc5ccc6ocnc6c5)c4c3)c(F)c2)CCN1] | 4-(4-(4-(benzo[d]oxazol-5- ylamino)quinolin-6-yl)-3- fluorobenzyl)piperazin-2- one |
| 260 | | 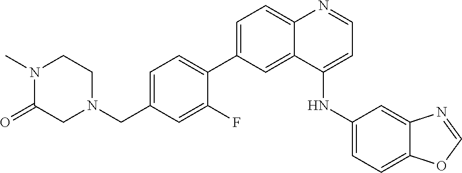 [SMILES: CN1CCN(Cc2ccc(-c3ccc4nccc(Nc5ccc6ocnc6c5)c4c3)c(F)c2)CC1=O] | 4-(4-(4-(benzo[d]oxazol-5- ylamino)quinolin-6-yl)-3- fluorobenzyl)-1- methylpiperazin-2-one |
| 261 | | 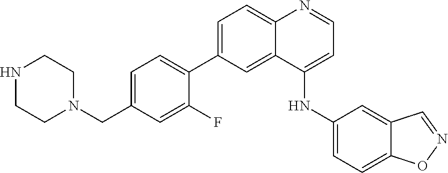 [SMILES: Fc1cc(CN2CCNCC2)ccc1-c1ccc2nccc(Nc3ccc4oncc4c3)c2c1] | N-(6-(2-fluoro-4- (piperazin-1- ylmethyl)phenyl)quinolin- 4-yl)benzo[d]isoxazol-5- amine |
| 262 (also referred to as Com- pound 289) | 89 | 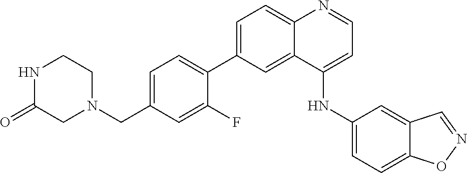 [SMILES: O=C1CN(Cc2ccc(-c3ccc4nccc(Nc5ccc6oncc6c5)c4c3)c(F)c2)CCN1] | 4-(4-(4-(benzo[d]isoxazol- 5-ylamino)quinolin-6-yl)- 3-fluorobenzyl)piperazin- 2-one |
| 263 | | 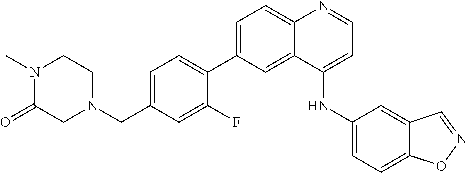 [SMILES: CN1CCN(Cc2ccc(-c3ccc4nccc(Nc5ccc6oncc6c5)c4c3)c(F)c2)CC1=O] | 4-(4-(4-(benzo[d]isoxazol- 5-ylamino)quinolin-6-yl)- 3-fluorobenzyl)-1- methylpiperazin-2-one |
| 264 (also referred to as Com- pound 386) | 79 | 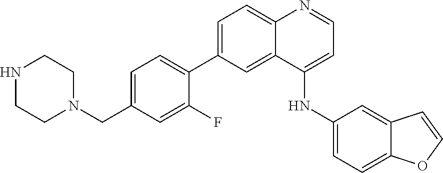 [SMILES: Fc1cc(CN2CCNCC2)ccc1-c1ccc2nccc(Nc3ccc4occc4c3)c2c1] | N-(benzofuran-5-yl)-6-(2- fluoro-4-(piperazin-1- ylmethyl)phenyl)quinolin- 4-amine |
| 265 (also referred to as Com- pound 287) | 87 | 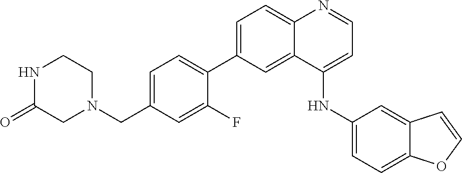 [SMILES: O=C1CN(Cc2ccc(-c3ccc4nccc(Nc5ccc6occc6c5)c4c3)c(F)c2)CCN1] | 4-(4-(4-(benzofuran-5- ylamino)quinolin-6-yl)-3- fluorobenzyl)piperazin-2- one |
| 266 | | 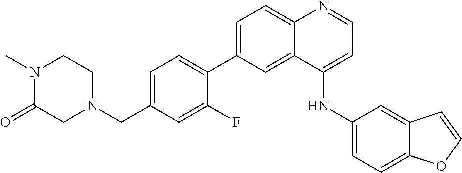 [SMILES: CN1CCN(Cc2ccc(-c3ccc4nccc(Nc5ccc6occc6c5)c4c3)c(F)c2)CC1=O] | 4-(4-(4-(benzofuran-5- ylamino)quinolin-6-yl)-3- fluorobenzyl)-1- methylpiperazin-2-one |
| 267 (also referred to as Com- pound 388) | 81 | 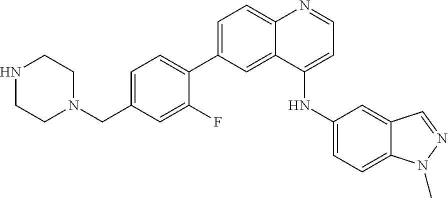 [SMILES: Cn1ncc2cc(Nc3ccnc4ccc(-c5ccc(CN6CCNCC6)cc5F)cc34)ccc21] | 6-(2-fluoro-4-(piperazin-1- ylmethyl)phenyl)-N-(1- methyl-1H-indazol-5- yl)quinolin-4-amine |
| 268 (also referred to as Com- pound 288) | 88 | 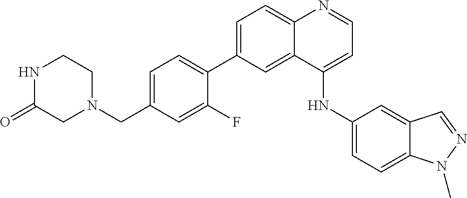 [SMILES: Cn1ncc2cc(Nc3ccnc4ccc(-c5ccc(CN6CCNC(=O)C6)cc5F)cc34)ccc21] | 4-(3-fluoro-4-(4-((1- methyl-1H-indazol-5- yl)amino)quinolin-6- yl)benzyl)piperazin-2-one |
| 269 | | 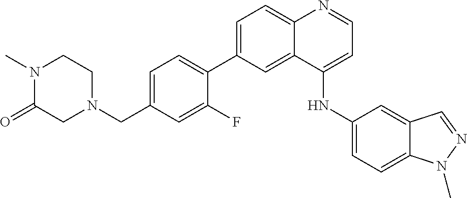 [SMILES: CN1CCN(Cc2ccc(-c3ccc4nccc(Nc5ccc6c(cnn6C)c5)c4c3)c(F)c2)CC1=O] | 4-(3-fluoro-4-(4-((1- methyl-1H-indazol-5- yl)amino)quinolin-6- yl)benzyl)-1- methylpiperazin-2-one |
| 270 (also referred to as Com- pound 387) | 80 | 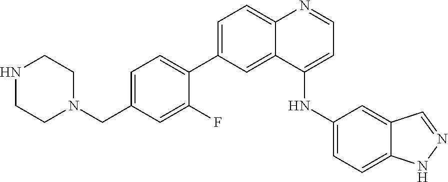 [SMILES: Fc1cc(CN2CCNCC2)ccc1-c1ccc2nccc(Nc3ccc4[nH]ncc4c3)c2c1] | 6-(2-fluoro-4-(piperazin-1- ylmethyl)phenyl)-N-(1H- indazol-5-yl)quinolin-4- amine |
| 271 | 71 | 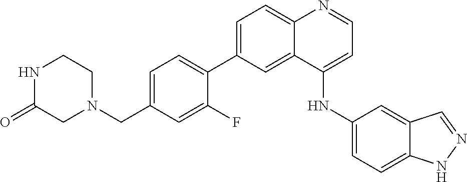 [SMILES: O=C1CN(Cc2ccc(-c3ccc4nccc(Nc5ccc6[nH]ncc6c5)c4c3)c(F)c2)CCN1] | 4-(4-(4-((1H-indazol-5- yl)amino)quinolin-6-yl)-3- fluorobenzyl)piperazin-2- one |
| 272 | | 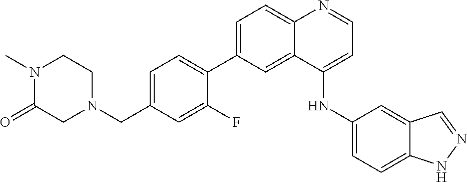 [SMILES: CN1CCN(Cc2ccc(-c3ccc4nccc(Nc5ccc6[nH]ncc6c5)c4c3)c(F)c2)CC1=O] | 4-(4-(4-((1H-indazol-5- yl)amino)quinolin-6-yl)-3- fluorobenzyl)-1- methylpiperazin-2-one |
| 273 (also referred to as Com- pound 79) | 67 | 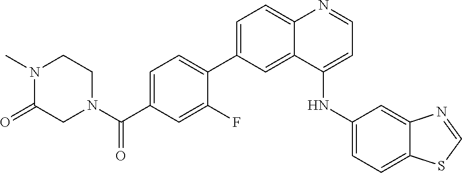 [SMILES: CN1CCN(C(=O)c2ccc(-c3ccc4nccc(Nc5ccc6scnc6c5)c4c3)c(F)c2)CC1=O] | 4-(4-(4-(benzo[d]thiazol-5- ylamino)quinolin-6-yl)-3- fluorobenzoyl)-1- methylpiperazin-2-one |
| 274 (also referred to as Com- pound 136) | 68 | 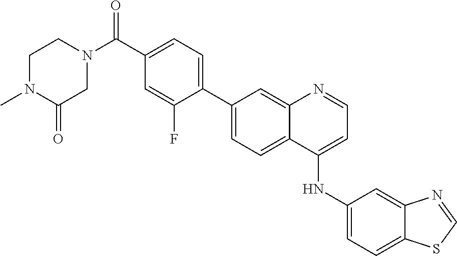 [SMILES: CN1CCN(C(=O)c2ccc(-c3ccc4c(Nc5ccc6scnc6c5)ccnc4c3)c(F)c2)CC1=O] | 4-(4-(4-(benzo[d]thiazol-5- ylamino)quinolin-7-yl)-3- fluorobenzoyl)-1- methylpiperazin-2-one |
| 275 | 69 | 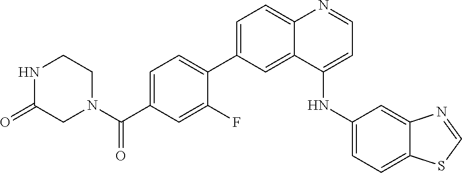 [SMILES: O=C1CN(C(=O)c2ccc(-c3ccc4nccc(Nc5ccc6scnc6c5)c4c3)c(F)c2)CCN1] | 4-(4-(4-(benzo[d]thiazol-5- ylamino)quinolin-6-yl)-3- fluorobenzoyl)piperazin-2- one |
| 276 | 70 | 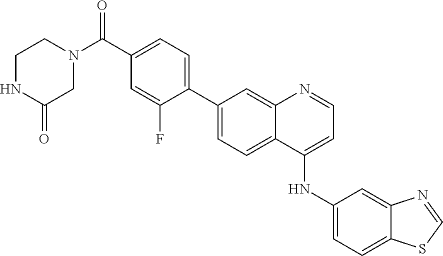 [SMILES: O=C1CN(C(=O)c2ccc(-c3ccc4c(Nc5ccc6scnc6c5)ccnc4c3)c(F)c2)CCN1] | 4-(4-(4-(benzo[d]thiazol-5- ylamino)quinolin-7-yl)-3- fluorobenzoyl)piperazin-2- one |
| 277 | | 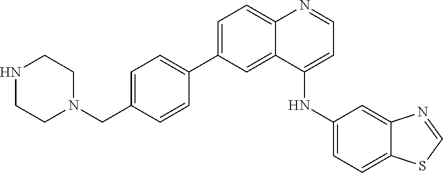 [SMILES: c1cc(Nc2ccc3scnc3c2)c2cc(-c3ccc(CN4CCNCC4)cc3)ccc2n1] | N-(6-(4-(piperazin-1- ylmethyl)phenyl)quinolin- 4-yl)benzo[d]thiazol-5- amine |
| 278 | | 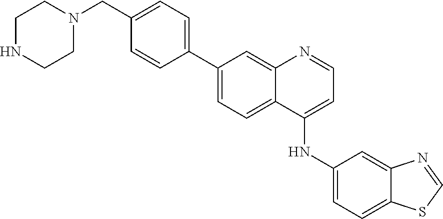 [SMILES: c1cc(Nc2ccc3scnc3c2)c2ccc(-c3ccc(CN4CCNCC4)cc3)cc2n1] | N-(7-(4-(piperazin-1- ylmethyl)phenyl)quinolin- 4-yl)benzo[d]thiazol-5- amine |
| 279 | | 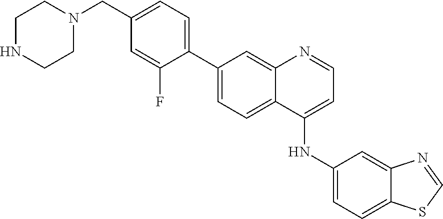 [SMILES: Fc1cc(CN2CCNCC2)ccc1-c1ccc2c(Nc3ccc4scnc4c3)ccnc2c1] | N-(7-(2-fluoro-4- (piperazin-1- ylmethyl)phenyl)quinolin- 4-yl)benzo[d]thiazol-5- amine |
| 280 (also referred to as Com- pound 281) | 77 | 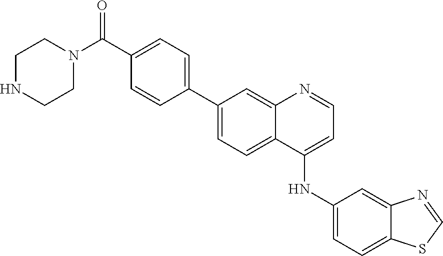 [SMILES: O=C(c1ccc(-c2ccc3c(Nc4ccc5scnc5c4)ccnc3c2)cc1)N1CCNCC1] | (4-(4-(benzo[d]thiazol-5- ylamino)quinolin-7- yl)phenyl)(piperazin-1- yl)methanone |
| 281 (also referred to as Com- pound 280) | 77 | 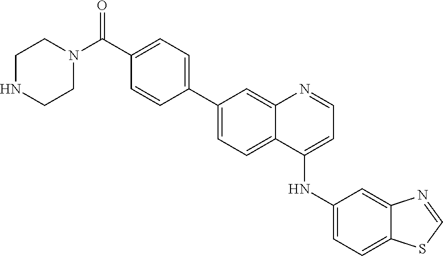 [SMILES: O=C(c1ccc(-c2ccc3c(Nc4ccc5scnc5c4)ccnc3c2)cc1)N1CCNCC1] | (4-(4-(benzo[d]thiazol-5- ylamino)quinolin-7- yl)phenyl)(piperazin-1- yl)methanone |
| 282 | 82 | 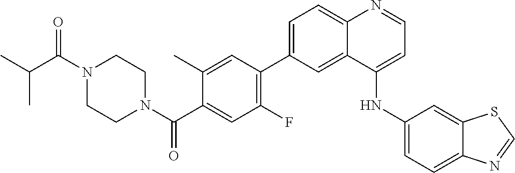 [SMILES: Cc1cc(-c2ccc3nccc(Nc4ccc5ncsc5c4)c3c2)c(F)cc1C(=O)N1CCN(C(=O)C(C)C)CC1] | 1-(4-(4-(4- (benzo[d]thiazol-6- ylamino)quinolin-6-yl)-5- fluoro-2- methylbenzoyl)piperazin- 1-yl)-2-methylpropan-1- one |
| 283 | 83 | 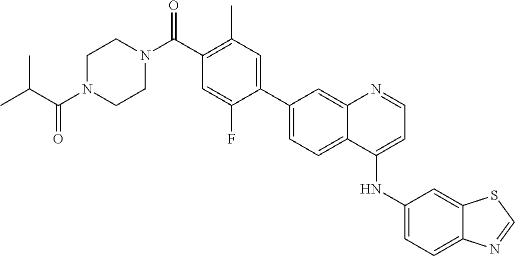 [SMILES: Cc1cc(-c2ccc3c(Nc4ccc5ncsc5c4)ccnc3c2)c(F)cc1C(=O)N1CCN(C(=O)C(C)C)CC1] | 1-(4-(4-(4- (benzo[d]thiazol-6- ylamino)quinolin-7-yl)-5- fluoro-2- methylbenzoyl)piperazin- 1-yl)-2-methylpropan-1- one |
| 284 | 84 | 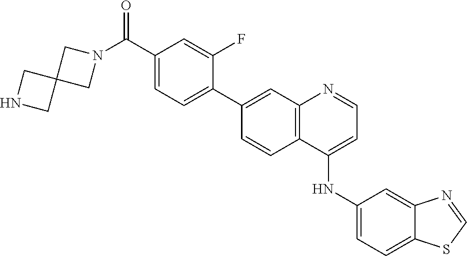 [SMILES: O=C(c1ccc(-c2ccc3c(Nc4ccc5scnc5c4)ccnc3c2)c(F)c1)N1CC2(CNC2)C1] | (4-(4-(benzo[d]thiazol-5- ylamino)quinolin-7-yl)-3- fluorophenyl)(2,6- diazaspiro[3.3]heptan-2- yl)methanone |
| 285 | 85 | 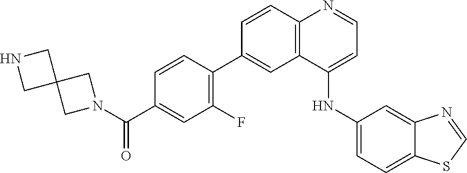 [SMILES: O=C(c1ccc(-c2ccc3nccc(Nc4ccc5scnc5c4)c3c2)c(F)c1)N1CC2(CNC2)C1] | (4-(4-(benzo[d]thiazol-5- ylamino)quinolin-6-yl)-3- fluorophenyl)(2,6- diazaspiro[3.3]heptan-2- yl)methanone |
| 286 | 86 | 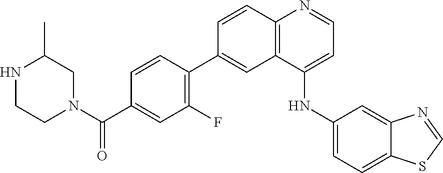 [SMILES: CC1CN(C(=O)c2ccc(-c3ccc4nccc(Nc5ccc6scnc6c5)c4c3)c(F)c2)CCN1] | (4-(4-(benzo[d]thiazol-5- ylamino)quinolin-6-yl)-3- fluorophenyl)(3- methylpiperazin-1- yl)methanone |
| 287 (also referred to as Com- pound 265) | 87 | 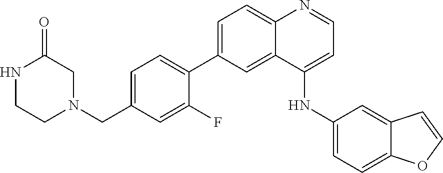 [SMILES: O=C1CN(Cc2ccc(-c3ccc4nccc(Nc5ccc6occc6c5)c4c3)c(F)c2)CCN1] | 4-(4-(4-(benzofuran-5- ylamino)quinolin-6-yl)-3- fluorobenzyl)piperazin-2- one |
| 288 (also referred to as Com- pound 268) | 88 | 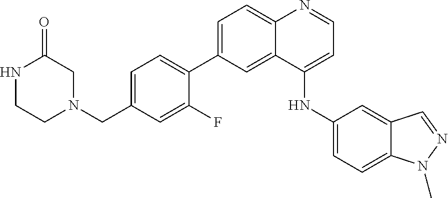 [SMILES: Cn1ncc2cc(Nc3ccnc4ccc(-c5ccc(CN6CCNC(=O)C6)cc5F)cc34)ccc21] | 4-(3-fluoro-4-(4-((1- methyl-1H-indazol-5- yl)amino)quinolin-6- yl)benzyl)piperazin-2-one |
| 289 (also referred to as Com- pound 262) | 89 | 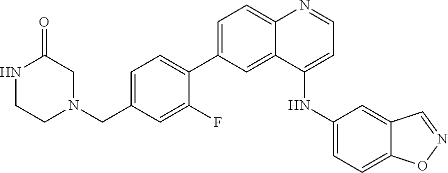 [SMILES: O=C1CN(Cc2ccc(-c3ccc4nccc(Nc5ccc6oncc6c5)c4c3)c(F)c2)CCN1] | 4-(4-(4-(benzo[d]isoxazol- 5-ylamino)quinolin-6-yl)- 3-fluorobenzyl)piperazin- 2-one |
| 290 | 90 | 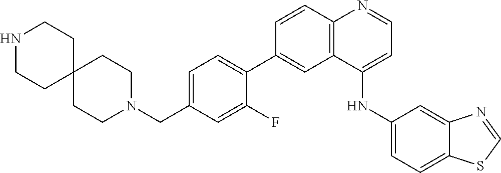 [SMILES: Fc1cc(CN2CCC3(CCNCC3)CC2)ccc1-c1ccc2nccc(Nc3ccc4scnc4c3)c2c1] | N-(6-(4-((3,9- diazaspiro[5.5]undecan-3- yl)methyl)-2- fluorophenyl)quinolin-4- yl)benzo[d]thiazol-5- amine |
| 291 | 91 | 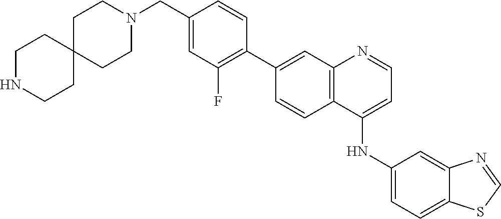 [SMILES: Fc1cc(CN2CCC3(CCNCC3)CC2)ccc1-c1ccc2c(Nc3ccc4scnc4c3)ccnc2c1] | N-(7-(4-((3,9- diazaspiro[5.5]undecan-3- yl)methyl)-2- fluorophenyl)quinolin-4- yl)benzo[d]thiazol-5- amine |
| 292 | 92 | 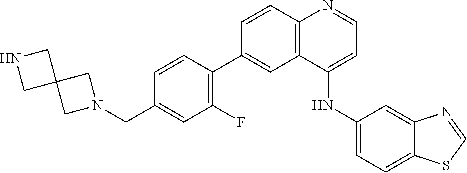 [SMILES: Fc1cc(CN2CC3(CNC3)C2)ccc1-c1ccc2nccc(Nc3ccc4scnc4c3)c2c1] | N-(6-(4-((2,6- diazaspiro[3.3]heptan-2- yl)methyl)-2- fluorophenyl)quinolin-4- yl)benzo[d]thiazol-5- amine |
| 293 | 93 | 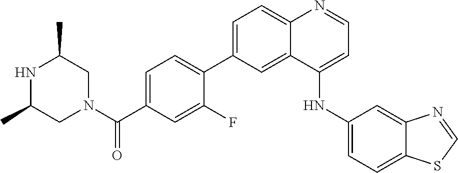 [SMILES: C[C@@H]1CN(C(=O)c2ccc(-c3ccc4nccc(Nc5ccc6scnc6c5)c4c3)c(F)c2)C[C@H](C)N1] | (4-(4-(benzo[d]thiazol-5- ylamino)quinolin-6-yl)-3- fluorophenyl)(3,5- dimethylpiperazin-1- yl)methanone or (4-(4-(benzo[d]thiazol-5- ylamino)quinolin-6-yl)-3- fluorophenyl)((3S,5R)-3,5- dimethylpiperazin-1- yl)methanone |
| 294 | 94 | 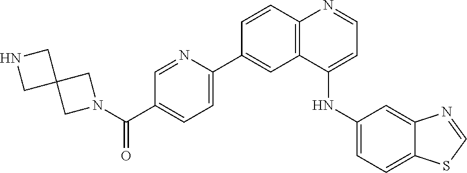 [SMILES: O=C(c1ccc(-c2ccc3nccc(Nc4ccc5scnc5c4)c3c2)nc1)N1CC2(CNC2)C1] | (6-(4-(benzo[d]thiazol-5- ylamino)quinolin-6- yl)pyridin-3-yl)(2,6- diazaspiro[3.3]heptan-2- yl)methanone |
| 295 (also referred to as Com- pound 74 and Com- pound 254) | 95 | 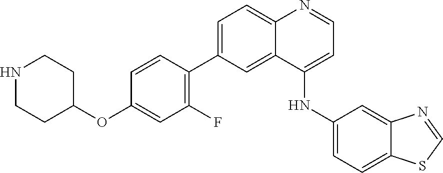 [SMILES: Fc1cc(OC2CCNCC2)ccc1-c1ccc2nccc(Nc3ccc4scnc4c3)c2c1] | N-(6-(2-fluoro-4- (piperidin-4- yloxy)phenyl)quinolin-4- yl)benzo[d]thiazol-5- amine |
| 296 | 96 | 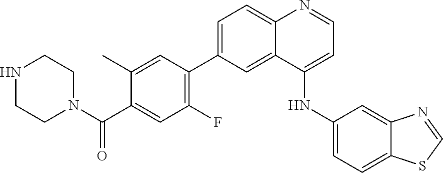 [SMILES: Cc1cc(-c2ccc3nccc(Nc4ccc5scnc5c4)c3c2)c(F)cc1C(=O)N1CCNCC1] | (4-(4-(benzo[d]thiazol-5- ylamino)quinolin-6-yl)-5- fluoro-2- methylphenyl)(piperazin- 1-yl)methanone |
| 297 | 97 | 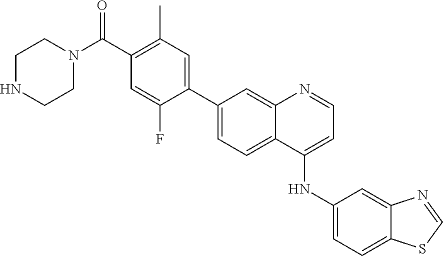 [SMILES: Cc1cc(-c2ccc3c(Nc4ccc5scnc5c4)ccnc3c2)c(F)cc1C(=O)N1CCNCC1] | (4-(4-(benzo[d]thiazol-5- ylamino)quinolin-7-yl)-5- fluoro-2- methylphenyl)(piperazin- 1-yl)methanone |
| 298 | 98 | 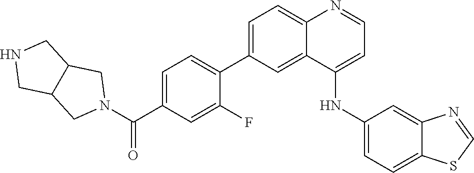 [SMILES: O=C(c1ccc(-c2ccc3nccc(Nc4ccc5scnc5c4)c3c2)c(F)c1)N1CC2CNCC2C1] | (4-(4-(benzo[d]thiazol-5- ylamino)quinolin-6-yl)-3- fluorophenyl)(hexahydro- pyrrolo[3,4-c]pyrrol-2(1H)- yl)methanone |
| 299 | 99 | 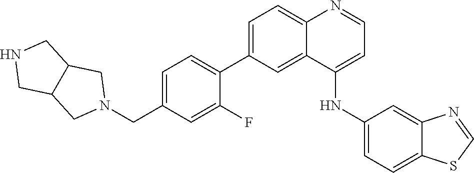 [SMILES: Fc1cc(CN2CC3CNCC3C2)ccc1-c1ccc2nccc(Nc3ccc4scnc4c3)c2c1] | N-(6-(2-fluoro-4- ((hexahydropyrrolo[3,4- c]pyrrol-2(1H)- yl)methyl)phenyl)quinolin- 4-yl)benzo[d]thiazol-5- amine |
| 300 | 100 | 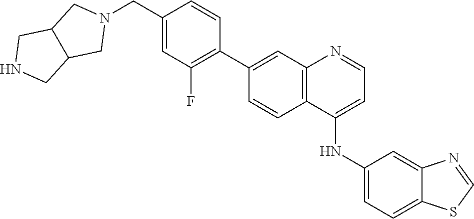 [SMILES: Fc1cc(CN2CC3CNCC3C2)ccc1-c1ccc2c(Nc3ccc4scnc4c3)ccnc2c1] | N-(7-(2-fluoro-4- ((hexahydropyrrolo[3,4- c]pyrrol-2(1H)- yl)methyl)phenyl)quinolin- 4-yl)benzo[d]thiazol-5- amine |
| 301 | 101 | 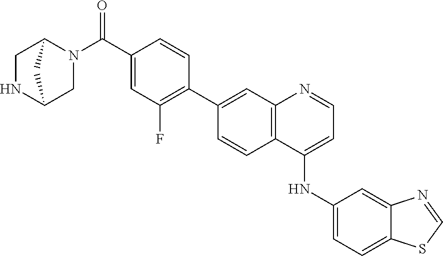 [SMILES: O=C(c1ccc(-c2ccc3c(Nc4ccc5scnc5c4)ccnc3c2)c(F)c1)N1C[C@@H]2C[C@H]1CN2] | (4-(4-(benzo[d]thiazol-5- ylamino)quinolin-7-yl)-3- fluorophenyl)(2,5- diazabicyclo[2.2.1]heptan- 2-yl)methanone or (4-(4-(benzo[d]thiazol-5- ylamino)quinolin-7-yl)-3- fluorophenyl)((1S,4S)-2,5- diazabicyclo[2.2.1]heptan- 2-yl)methanone |
| 302 | 102 | 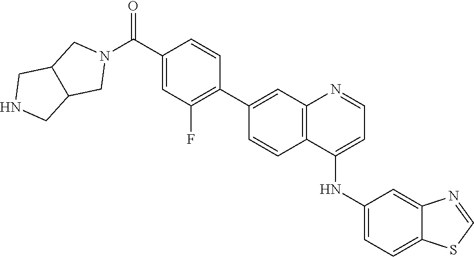 [SMILES: O=C(c1ccc(-c2ccc3c(Nc4ccc5scnc5c4)ccnc3c2)c(F)c1)N1CC2CNCC2C1] | (4-(4-(benzo[d]thiazol-5- ylamino)quinolin-7-yl)-3- fluorophenyl)(hexahydro- pyrrolo[3,4-c]pyrrol-2(1H)- yl)methanone |
| 303 | 103 | 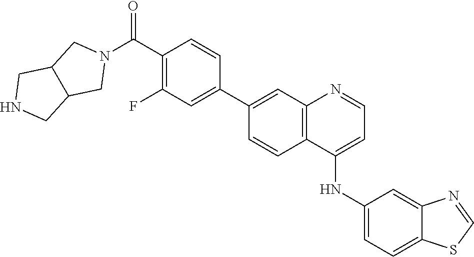 [SMILES: O=C(c1ccc(-c2ccc3c(Nc4ccc5scnc5c4)ccnc3c2)cc1F)N1CC2CNCC2C1] | (4-(4-(benzo[d]thiazol-5- ylamino)quinolin-7-yl)-2- fluorophenyl)(hexahydro- pyrrolo[3,4-c]pyrrol-2(1H)- yl)methanone |
| 304 | 104 | 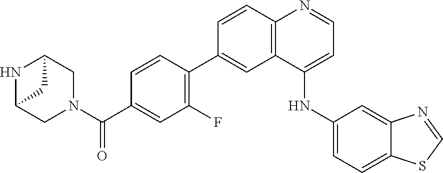 [SMILES: O=C(c1ccc(-c2ccc3nccc(Nc4ccc5scnc5c4)c3c2)c(F)c1)N1C[C@H]2C[C@@H](C1)N2] | (4-(4-(benzo[d]thiazol-5- ylamino)quinolin-6-yl)-3- fluorophenyl)(-3,6- diazabicyclo[3.1.1]heptan- 3-yl)methanone or (4-(4-(benzo[d]thiazol-5- ylamino)quinolin-6-yl)-3- fluorophenyl)((1R,5S)-3,6- diazabicyclo[3.1.1]heptan- 3-yl)methanone |
| 305 | 105 | 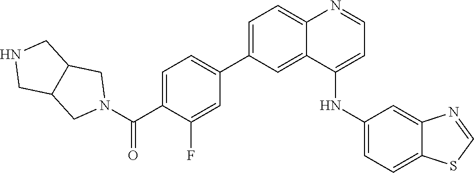 [SMILES: O=C(c1ccc(-c2ccc3nccc(Nc4ccc5scnc5c4)c3c2)cc1F)N1CC2CNCC2C1] | (4-(4-(benzo[d]thiazol-5- ylamino)quinolin-6-yl)-2- fluorophenyl)(hexahydro- pyrrolo[3,4-c]pyrrol-2(1H)- yl)methanone |
| 306 | 106 | 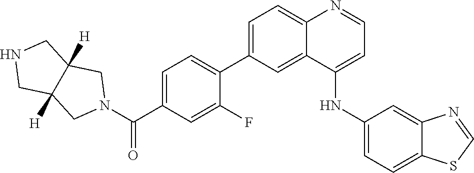 [SMILES: O=C(c1ccc(-c2ccc3nccc(Nc4ccc5scnc5c4)c3c2)c(F)c1)N1C[C@H]2CNC[C@H]2C1] | (4-(4-(benzo[d]thiazol-5- ylamino)quinolin-6-yl)-3- fluorophenyl)(hexahydro- pyrrolo[3,4-c]pyrrol-2(1H)- yl)methanone or (4-(4-(benzo[d]thiazol-5- ylamino)quinolin-6-yl)-3- fluorophenyl)((3aR,6aS)- hexahydropyrrolo[3,4- c]pyrrol-2(1H)- |
| | | | yl)methanone |
| 307 | 107 | 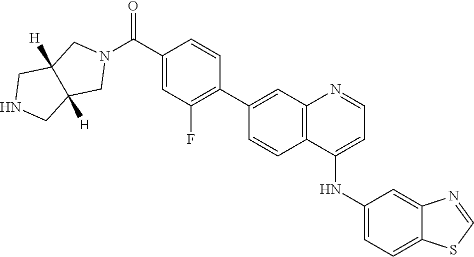 [SMILES: O=C(c1ccc(-c2ccc3c(Nc4ccc5scnc5c4)ccnc3c2)c(F)c1)N1C[C@H]2CNC[C@H]2C1] | (4-(4-(benzo[d]thiazol-5- ylamino)quinolin-7-yl)-3- fluorophenyl)(hexahydro- pyrrolo[3,4-c]pyrrol-2(1H)- yl)methanone or (4-(4-(benzo[d]thiazol-5- ylamino)quinolin-7-yl)-3- fluorophenyl)((3aR,6aS)- hexahydropyrrolo[3,4- c]pyrrol-2(1H)- yl)methanone |
| 308 | 108 | 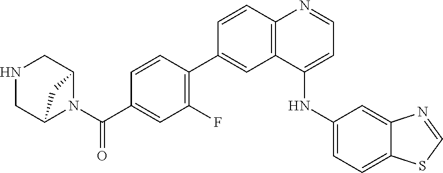 [SMILES: O=C(c1ccc(-c2ccc3nccc(Nc4ccc5scnc5c4)c3c2)c(F)c1)N1[C@@H]2CNC[C@H]1C2] | (4-(4-(benzo[d]thiazol-5- ylamino)quinolin-6-yl)-3- fluorophenyl)(3,6- diazabicyclo[3.1.1]heptan- 6-yl)methanone or (4-(4-(benzo[d]thiazol-5- ylamino)quinolin-6-yl)-3- fluorophenyl)((1R,5S)-3,6- diazabicyclo[3.1.1]heptan- 6-yl)methanone |
| 309 | 109 | 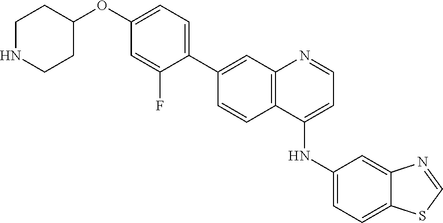 [SMILES: Fc1cc(OC2CCNCC2)ccc1-c1ccc2c(Nc3ccc4scnc4c3)ccnc2c1] | N-(7-(2-fluoro-4- (piperidin-4- yloxy)phenyl)quinolin-4- yl)benzo[d]thiazol-5-amine |
| 310 | 110 | 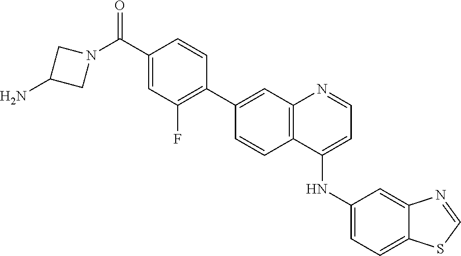 [SMILES: NC1CN(C(=O)c2ccc(-c3ccc4c(Nc5ccc6scnc6c5)ccnc4c3)c(F)c2)C1] | (3-aminoazetidin-1-yl)(4- (4-(benzo[d]thiazol-5- ylamino)quinolin-7-yl)-3- fluorophenyl)methanone |
| 311 | 111 | 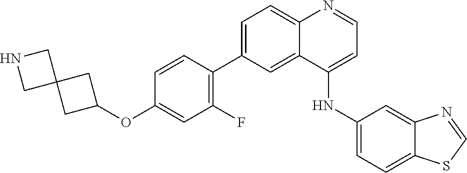 [SMILES: Fc1cc(OC2CC3(CNC3)C2)ccc1-c1ccc2nccc(Nc3ccc4scnc4c3)c2c1] | N-(6-(4-((2- azaspiro[3.3]heptan-6- yl)oxy)-2- fluorophenyl)quinolin-4- yl)benzo[d]thiazol-5-amine |
| 312 | 112 | 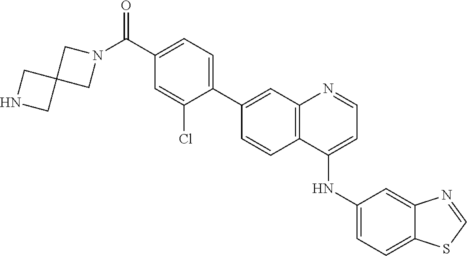 [SMILES: O=C(c1ccc(-c2ccc3c(Nc4ccc5scnc5c4)ccnc3c2)c(Cl)c1)N1CC2(CNC2)C1] | (4-(4-(benzo[d]thiazol-5- ylamino)quinolin-7-yl)-3- chlorophenyl)(2,6- diazaspiro[3.3]heptan-2- yl)methanone |
| 313 | 113 | 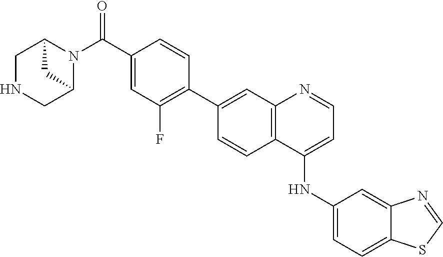 [SMILES: O=C(c1ccc(-c2ccc3c(Nc4ccc5scnc5c4)ccnc3c2)c(F)c1)N1[C@@H]2CNC[C@H]1C2] | (4-(4-(benzo[d]thiazol-5- ylamino)quinolin-7-yl)-3- fluorophenyl)(3,6- diazabicyclo[3.1.1]heptan- 6-yl)methanone or (4-(4-(benzo[d]thiazol-5- ylamino)quinolin-7-yl)-3- fluorophenyl)((1R,5S)-3,6- diazabicyclo[3.1.1]heptan- 6-yl)methanone |
| 314 | 114 | 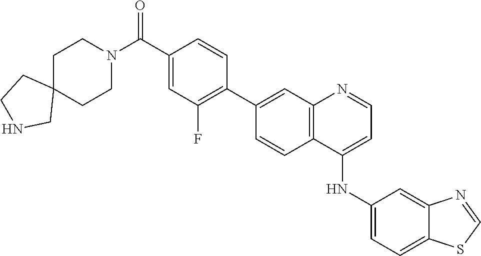 [SMILES: O=C(c1ccc(-c2ccc3c(Nc4ccc5scnc5c4)ccnc3c2)c(F)c1)N1CCC2(CCNC2)CC1] | (4-(4-(benzo[d]thiazol-5- ylamino)quinolin-7-yl)-3- fluorophenyl)(2,8- diazaspiro[4.5]decan-8- yl)methanone |
| 315 | 115 | 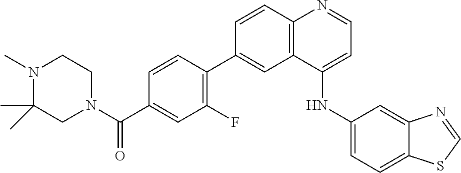 [SMILES: CN1CCN(C(=O)c2ccc(-c3ccc4nccc(Nc5ccc6scnc6c5)c4c3)c(F)c2)CC1(C)C] | (4-(4-(benzo[d]thiazol-5- ylamino)quinolin-6-yl)-3- fluorophenyl)(3,3,4- trimethylpiperazin-1- yl)methanone |
| 316 | 116 | 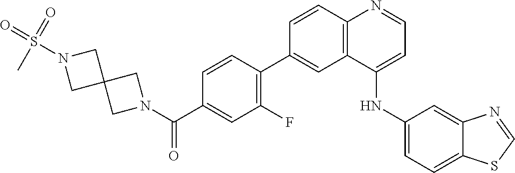 [SMILES: CS(=O)(=O)N1CC2(CN(C(=O)c3ccc(-c4ccc5nccc(Nc6ccc7scnc7c6)c5c4)c(F)c3)C2)C1] | (4-(4-(benzo[d]thiazol-5- ylamino)quinolin-6-yl)-3- fluorophenyl)(6- (methylsulfonyl)-2,6- diazaspiro[3.3]heptan-2- yl)methanone |
| 317 (also referred to as Com- pound 132) | 117 | 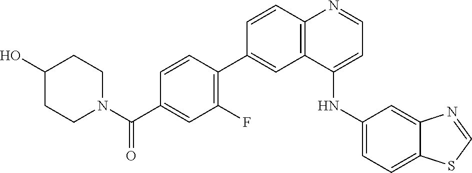 [SMILES: O=C(c1ccc(-c2ccc3nccc(Nc4ccc5scnc5c4)c3c2)c(F)c1)N1CCC(O)CC1] | (4-(4-(benzo[d]thiazol-5- ylamino)quinolin-6-yl)-3- fluorophenyl)(4- hydroxypiperidin-1- yl)methanone |
| 318 | 118 | 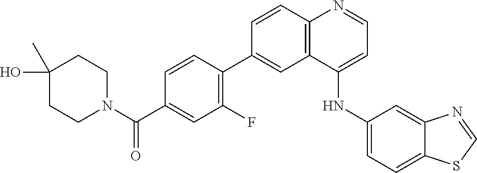 [SMILES: CC1(O)CCN(C(=O)c2ccc(-c3ccc4nccc(Nc5ccc6scnc6c5)c4c3)c(F)c2)CC1] | (4-(4-(benzo[d]thiazol-5- ylamino)quinolin-6-yl)-3- fluorophenyl)(4-hydroxy- 4-methylpiperidin-1- yl)methanone |
| 319 | 119 | 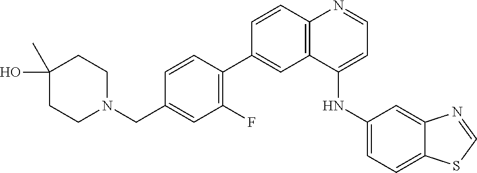 [SMILES: CC1(O)CCN(Cc2ccc(-c3ccc4nccc(Nc5ccc6scnc6c5)c4c3)c(F)c2)CC1] | 1-(4-(4-(benzo[d]thiazol-5- ylamino)quinolin-6-yl)-3- fluorobenzyl)-4- methylpiperidin-4-ol |
| 320 | 120 | 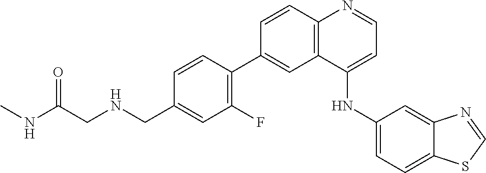 [SMILES: CNC(=O)CNCc1ccc(-c2ccc3nccc(Nc4ccc5scnc5c4)c3c2)c(F)c1] | 2-((4-(4-(benzo[d]thiazol- 5-ylamino)quinolin-6-yl)- 3-fluorobenzyl)amino)-N- methylacetamide |
| 321 | 121 | 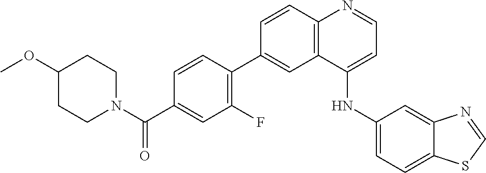 [SMILES: COC1CCN(C(=O)c2ccc(-c3ccc4nccc(Nc5ccc6scnc6c5)c4c3)c(F)c2)CC1] | (4-(4-(benzo[d]thiazol-5- ylamino)quinolin-6-yl)-3- fluorophenyl)(4- methoxypiperidin-1- yl)methanone |
| 322 | 122 | 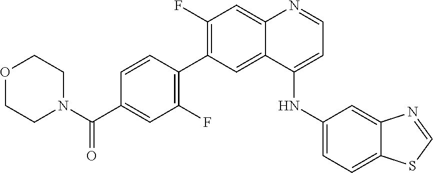 [SMILES: O=C(c1ccc(-c2cc3c(Nc4ccc5scnc5c4)ccnc3cc2F)c(F)c1)N1CCOCC1] | (4-(4-(benzo[d]thiazol-5- ylamino)-7-fluoroquinolin- 6-yl)-3- fluorophenyl)(morpholino) methanone |
| 323 | 123 | 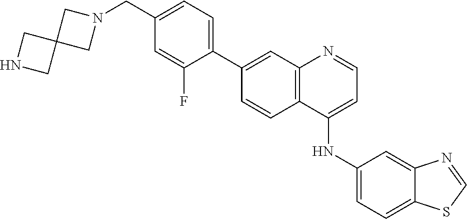 [SMILES: Fc1cc(CN2CC3(CNC3)C2)ccc1-c1ccc2c(Nc3ccc4scnc4c3)ccnc2c1] | N-(7-(4-((2,6- diazaspiro[3.3]heptan-2- yl)methyl)-2- fluorophenyl)quinolin-4- yl)benzo[d]thiazol-5-amine |
| 324 | 124 | 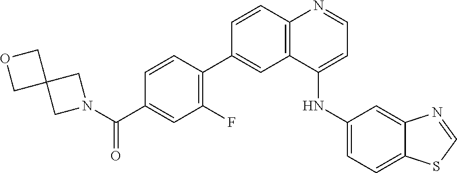 [SMILES: O=C(c1ccc(-c2ccc3nccc(Nc4ccc5scnc5c4)c3c2)c(F)c1)N1CC2(COC2)C1] | (4-(4-(benzo[d]thiazol-5- ylamino)quinolin-6-yl)-3- fluorophenyl)(2-oxa-6- azaspiro[3.3]heptan-6- yl)methanone |
| 325 | 125 | 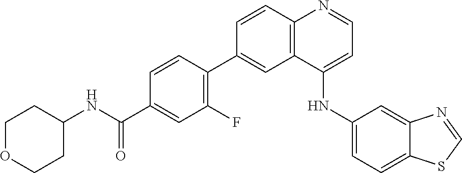 [SMILES: O=C(NC1CCOCC1)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N-(tetrahydro-2H- pyran-4-yl)benzamide |
| 326 | 126 | 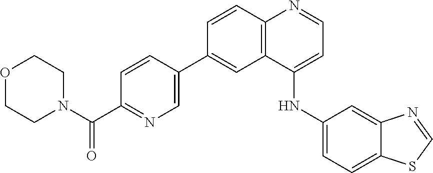 [SMILES: O=C(c1ccc(-c2ccc3nccc(Nc4ccc5scnc5c4)c3c2)cn1)N1CCOCC1] | (5-(4-(benzo[d]thiazol-5- ylamino)quinolin-6- yl)pyridin-2- yl)(morpholino)methanone |
| 327 | 127 | 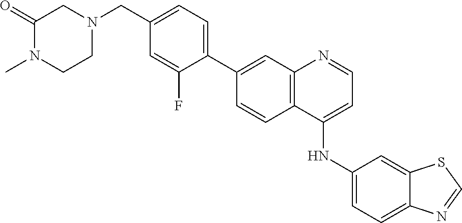 [SMILES: CN1CCN(Cc2ccc(-c3ccc4c(Nc5ccc6ncsc6c5)ccnc4c3)c(F)c2)CC1=O] | 4-(4-(4-(benzo[d]thiazol-5- ylamino)quinolin-7-yl)-3- fluorobenzyl)-1- methylpiperazin-2-one |
| 328 | | 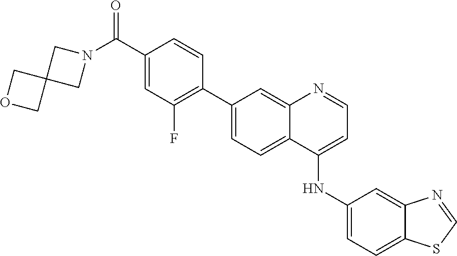 [SMILES: O=C(c1ccc(-c2ccc3c(Nc4ccc5scnc5c4)ccnc3c2)c(F)c1)N1CC2(COC2)C1] | (4-(4-(benzo[d]thiazol-5- ylamino)quinolin-7-yl)-3- fluorophenyl)(2-oxa-6- azaspiro[3.3]heptan-6- yl)methanone |
| 329 | | 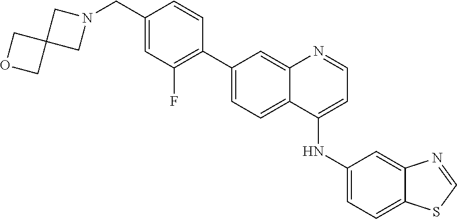 [SMILES: Fc1cc(CN2CC3(COC3)C2)ccc1-c1ccc2c(Nc3ccc4scnc4c3)ccnc2c1] | N-(7-(4-((2-oxa-6- azaspiro[3.3]heptan-6- yl)methyl)-2- fluorophenyl)quinolin-4- yl)benzo[d]thiazol-5-amine |
| 330 | | 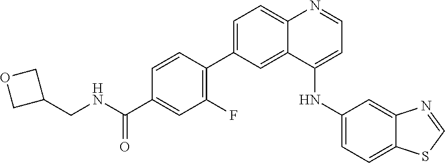 [SMILES: O=C(NCC1COC1)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N-(oxetan-3- ylmethyl)benzamide |
| 331 | | 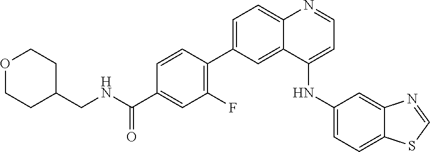 [SMILES: O=C(NCC1CCOCC1)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N-((tetrahydro-2H- pyran-4- yl)methyl)benzamide |
| 332 | | 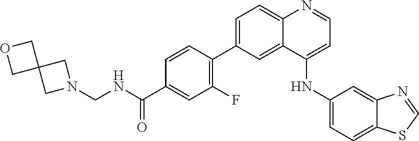 [SMILES: O=C(NCN1CC2(COC2)C1)c1ccc(-c2ccc3nccc(Nc4ccc5scnc5c4)c3c2)c(F)c1] | N-(6-(4-((2-oxa-6- azaspiro[3.3]heptan-6- yl)methyl)-2- fluorophenyl)quinolin-4- yl)benzo[d]thiazol-5-amine |
| 333 | | 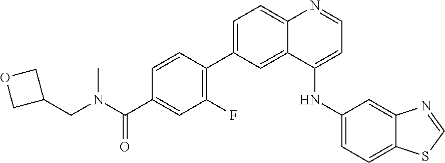 [SMILES: CN(CC1COC1)C(=O)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N-methyl-N- (oxetan-3- ylmethyl)benzamide |
| 334 | | 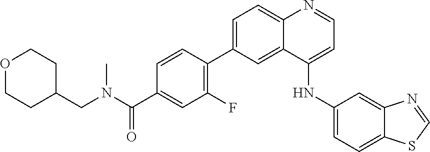 [SMILES: CN(CC1CCOCC1)C(=O)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N-methyl-N- ((tetrahydro-2H-pyran-4- yl)methyl)benzamide |
| 335 | | 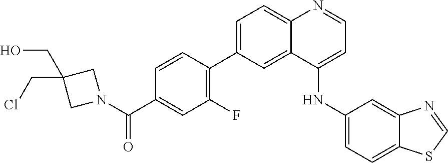 [SMILES: O=C(c1ccc(-c2ccc3nccc(Nc4ccc5scnc5c4)c3c2)c(F)c1)N1CC(CO)(CCl)C1] | (4-(4-(benzo[d]thiazol-5- ylamino)quinolin-6-yl)-3- fluorophenyl)(3- (chloromethyl)-3- (hydroxymethyl)azetidin- 1-yl)methanone |
| 336 | | 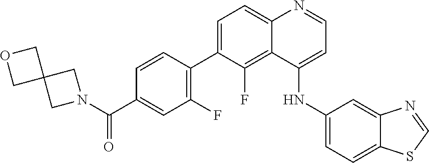 [SMILES: O=C(c1ccc(-c2ccc3nccc(Nc4ccc5scnc5c4)c3c2F)c(F)c1)N1CC2(COC2)C1] | (4-(4-(benzo[d]thiazol-5- ylamino)-5-fluoroquinolin- 6-yl)-3-fluorophenyl)(2- oxa-6-azaspiro[3.3]heptan- 6-yl)methanone |
| 337 | | 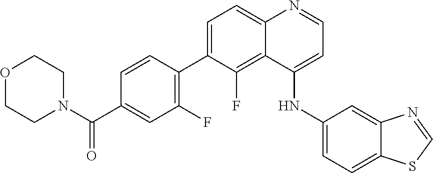 [SMILES: O=C(c1ccc(-c2ccc3nccc(Nc4ccc5scnc5c4)c3c2F)c(F)c1)N1CCOCC1] | (4-(4-(benzo[d]thiazol-5- ylamino)-5-fluoroquinolin- 6-yl)-3- fluorophenyl)(morpholino) methanone |
| 338 | | 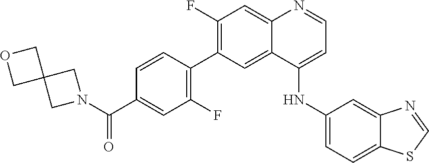 [SMILES: O=C(c1ccc(-c2cc3c(Nc4ccc5scnc5c4)ccnc3cc2F)c(F)c1)N1CC2(COC2)C1] | (4-(4-(benzo[d]thiazol-5- ylamino)-7-fluoroquinolin- 6-yl)-3-fluorophenyl)(2- oxa-6-azaspiro[3.3]heptan- 6-yl)methanone |
| 339 | | 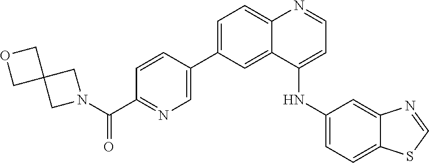 [SMILES: O=C(c1ccc(-c2ccc3nccc(Nc4ccc5scnc5c4)c3c2)cn1)N1CC2(COC2)C1] | (5-(4-(benzo[d]thiazol-5- ylamino)quinolin-6- yl)pyridin-2-yl)(2-oxa-6- azaspiro[3.3]heptan-6- yl)methanone |
| 340 | | 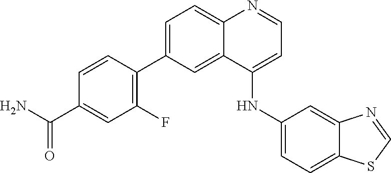 [SMILES: NC(=O)c1ccc(-c2ccc3nccc(Nc4ccc5scnc5c4)c3c2)c(F)c1] | 4-(4-(benzo[d]thiazol-5- ylamino)quinolin-6-yl)-3- fluorobenzamide |
| 341 (also referred to as Com- pound 85) | | 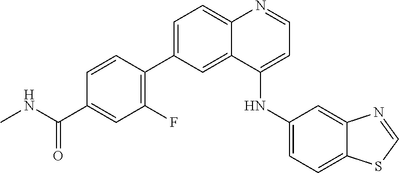 [SMILES: CNC(=O)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N-methylbenzamide |
| 342 (also referred to as Com- pound 245) | 62 | 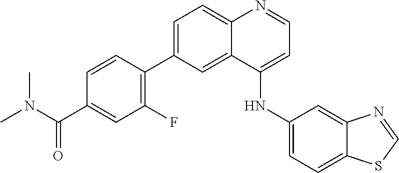 [SMILES: CN(C)C(=O)c1ccc(-c2ccc3nccc(Nc4ccc5scnc5c4)c3c2)c(F)c1] | 4-(4-(benzo[d]thiazol-5- ylamino)quinolin-6-yl)-3- fluoro-N,N- dimethylbenzamide |
| 343 | | 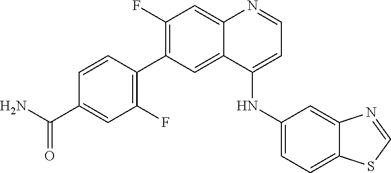 [SMILES: NC(=O)c1ccc(-c2cc3c(Nc4ccc5scnc5c4)ccnc3cc2F)c(F)c1] | 4-(4-(benzo[d]thiazol-5- ylamino)-7-fluoroquinolin- 6-yl)-3-fluorobenzamide |
| 344 | | 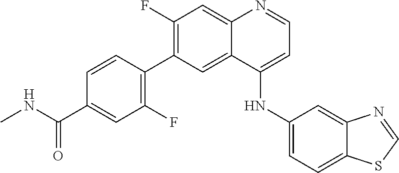 [SMILES: CNC(=O)c1ccc(-c2cc3c(Nc4ccc5scnc5c4)ccnc3cc2F)c(F)c1] | 4-(4-(benzo[d]thiazol-5- ylamino)-7-fluoroquinolin- 6-yl)-3-fluoro-N- methylbenzamide |
| 345 | | 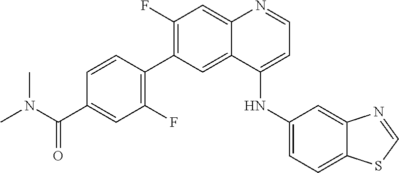 [SMILES: CN(C)C(=O)c1ccc(-c2cc3c(Nc4ccc5scnc5c4)ccnc3cc2F)c(F)c1] | 4-(4-(benzo[d]thiazol-5- ylamino)-7-fluoroquinolin- 6-yl)-3-fluoro-N,N- dimethylbenzamide |
| 346 | | 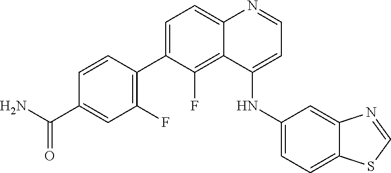 [SMILES: NC(=O)c1ccc(-c2ccc3nccc(Nc4ccc5scnc5c4)c3c2F)c(F)c1] | 4-(4-(benzo[d]thiazol-5- ylamino)-5-fluoroquinolin- 6-yl)-3-fluorobenzamide |
| 347 | | 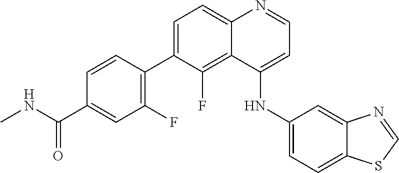 [SMILES: CNC(=O)c1ccc(-c2ccc3nccc(Nc4ccc5scnc5c4)c3c2F)c(F)c1] | 4-(4-(benzo[d]thiazol-5- ylamino)-5-fluoroquinolin- 6-yl)-3-fluoro-N- methylbenzamide |
| 348 | | 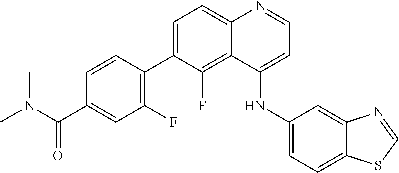 [SMILES: CN(C)C(=O)c1ccc(-c2ccc3nccc(Nc4ccc5scnc5c4)c3c2F)c(F)c1] | 4-(4-(benzo[d]thiazol-5- ylamino)-5-fluoroquinolin- 6-yl)-3-fluoro-N,N- dimethylbenzamide |
| 349 | | 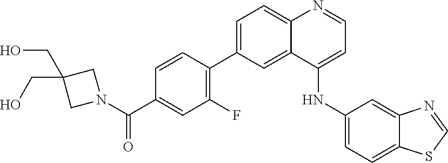 [SMILES: O=C(c1ccc(-c2ccc3nccc(Nc4ccc5scnc5c4)c3c2)c(F)c1)N1CC(CO)(CO)C1] | (4-(4-(benzo[d]thiazol-5- ylamino)quinolin-6-yl)-3- fluorophenyl)(3,3- bis(hydroxymethyl)azetidin- 1-yl)methanone |
| 350 | | 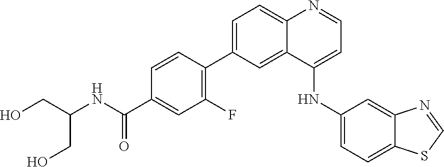 [SMILES: O=C(NC(CO)CO)c1ccc(-c2ccc3nccc(Nc4ccc5scnc5c4)c3c2)c(F)c1] | 4-(4-(benzo[d]thiazol-5- ylamino)quinolin-6-yl)-N- (1,3-dihydroxypropan-2- yl)-3-fluorobenzamide |
| 351 | | 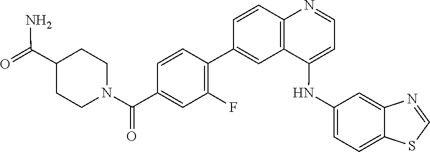 [SMILES: NC(=O)C1CCN(C(=O)c2ccc(-c3ccc4nccc(Nc5ccc6scnc6c5)c4c3)c(F)c2)CC1] | 1-(4-(4-(benzo[d]thiazol-5- ylamino)quinolin-6-yl)-3- fluorobenzoyl)piperidine- 4-carboxamide |
| 352 | | 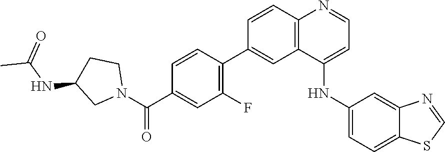 [SMILES: CC(=O)N[C@H]1CCN(C(=O)c2ccc(-c3ccc4nccc(Nc5ccc6scnc6c5)c4c3)c(F)c2)C1] | N-(1-(4-(4- (benzo[d]thiazol-5- ylamino)quinolin-6-yl)-3- fluorobenzoyl)pyrrolidin- 3-yl)acetamide or (S)-N-(1-(4-(4- (benzo[d]thiazol-5- ylamino)quinolin-6-yl)-3- fluorobenzoyl)pyrrolidin- |
| | | | 3-yl)acetamide |
| 353 | | 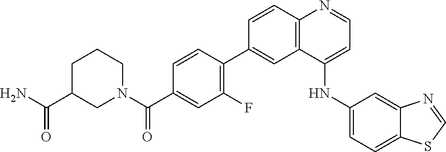 [SMILES: NC(=O)C1CCCN(C(=O)c2ccc(-c3ccc4nccc(Nc5ccc6scnc6c5)c4c3)c(F)c2)C1] | 1-(4-(4-(benzo[d]thiazol-5- ylamino)quinolin-6-yl)-3- fluorobenzoyl)piperidine- 3-carboxamide |
| 354 | | 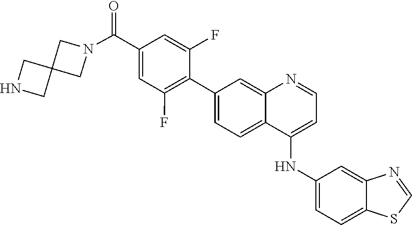 [SMILES: O=C(c1cc(F)c(-c2ccc3c(Nc4ccc5scnc5c4)ccnc3c2)c(F)c1)N1CC2(CNC2)C1] | (4-(4-(benzo[d]thiazol-5- ylamino)quinolin-7-yl)- 3,5-difluorophenyl)(2,6- diazaspiro[3.3]heptan-2- yl)methanone |
| 355 | | 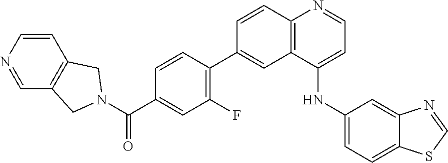 [SMILES: O=C(c1ccc(-c2ccc3nccc(Nc4ccc5scnc5c4)c3c2)c(F)c1)N1Cc2ccncc2C1] | (4-(4-(benzo[d]thiazol-5- ylamino)quinolin-6-yl)-3- fluorophenyl)(1,3-dihydro- 2H-pyrrolo[3,4-c]pyridin- 2-yl)methanone |
| 356 | | 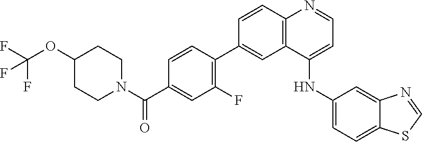 [SMILES: O=C(c1ccc(-c2ccc3nccc(Nc4ccc5scnc5c4)c3c2)c(F)c1)N1CCC(OC(F)(F)F)CC1] | (4-(4-(benzo[d]thiazol-5- ylamino)quinolin-6-yl)-3- fluorophenyl)(4- (trifluoromethoxy)piperidin- 1-yl)methanone |
| 357 | | 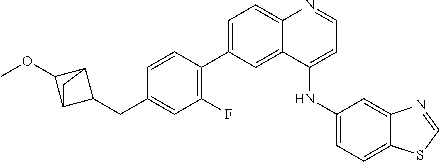 [SMILES: COC1C2CC1C2Cc1ccc(-c2ccc3nccc(Nc4ccc5scnc5c4)c3c2)c(F)c1] | N-(6-(2-fluoro-4-((4- methoxybicyclo[1.1.1] pentan-2- yl)methyl)phenyl)quinolin- 4-yl)benzo[d]thiazol-5- amine |
| 358 | | 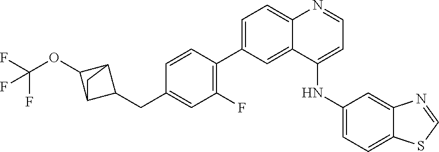 [SMILES: Fc1cc(CC2C3CC2C3OC(F)(F)F)ccc1-c1ccc2nccc(Nc3ccc4scnc4c3)c2c1] | N-(6-(2-fluoro-4-((4- (trifluoromethoxy)bicyclo [1.1.1]pentan-2- yl)methyl)phenyl)quinolin- 4-yl)benzo[d]thiazol-5- amine |
| 359 | | 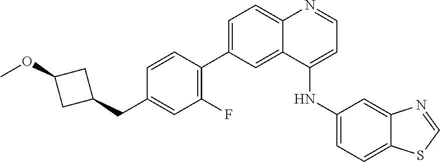 [SMILES: CO[C@H]1C[C@@H](Cc2ccc(-c3ccc4nccc(Nc5ccc6scnc6c5)c4c3)c(F)c2)C1] | N-(6-(2-fluoro-4-((3- methoxycyclobutyl)methyl) phenyl)quinolin-4- yl)benzo[d]thiazol-5-amine Or N-(6-(2-fluoro-4-(((1s,3r)-3- methoxycyclobutyl)methyl) phenyl)quinolin-4- yl)benzo[d]thiazol-5-amine |
| 360 | | 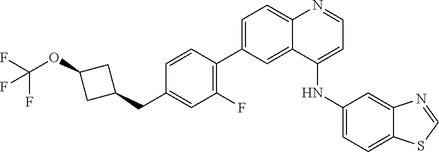 [SMILES: Fc1cc(C[C@H]2C[C@@H](OC(F)(F)F)C2)ccc1-c1ccc2nccc(Nc3ccc4scnc4c3)c2c1] | N-(6-(2-fluoro-4-((3- (trifluoromethoxy)cyclo- butyl)methyl)phenyl)quinolin- 4-yl)benzo[d]thiazol-5- amine or N-(6-(2-fluoro-4-(((1s,3r)-3- (trifluoromethoxy)cyclo- butyl)methyl)phenyl)quinolin- 4-yl)benzo[d]thiazol-5- amine |
| 361 | | 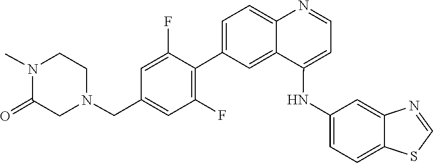 [SMILES: CN1CCN(Cc2cc(F)c(-c3ccc4nccc(Nc5ccc6scnc6c5)c4c3)c(F)c2)CC1=O] | 4-(4-(4-(benzo[d]thiazol-5- ylamino)quinolin-6-yl)- 3,5-difluorobenzyl)-1- methylpiperazin-2-one |
| 362 | | 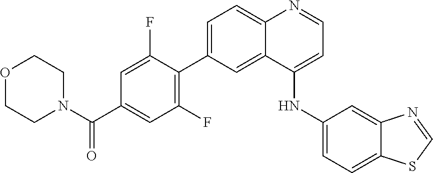 [SMILES: O=C(c1cc(F)c(-c2ccc3nccc(Nc4ccc5scnc5c4)c3c2)c(F)c1)N1CCOCC1] | (4-(4-(benzo[d]thiazol-5- ylamino)quinolin-6-yl)- 3,5- difluorophenyl)(morpholino) methanone |
| 363 | | 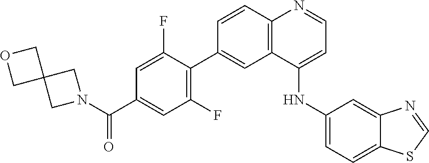 [SMILES: O=C(c1cc(F)c(-c2ccc3nccc(Nc4ccc5scnc5c4)c3c2)c(F)c1)N1CC2(COC2)C1] | (4-(4-(benzo[d]thiazol-5- ylamino)quinolin-6-yl)- 3,5-difluorophenyl)(2-oxa- 6-azaspiro[3.3]heptan-6- yl)methanone |
| 364 | | 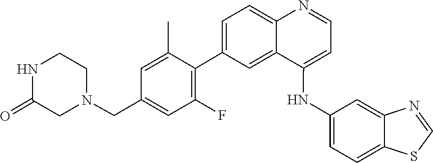 [SMILES: Cc1cc(CN2CCNC(=O)C2)cc(F)c1-c1ccc2nccc(Nc3ccc4scnc4c3)c2c1] | 4-(4-(4-(benzo[d]thiazol-5- ylamino)quinolin-6-yl)-3- fluoro-5- methylbenzyl)piperazin-2- one |
| 365 | | 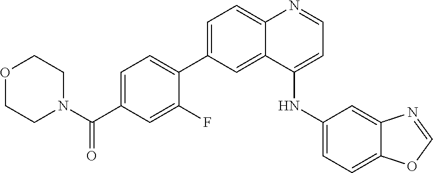 [SMILES: O=C(c1ccc(-c2ccc3nccc(Nc4ccc5ocnc5c4)c3c2)c(F)c1)N1CCOCC1] | (4-(4-(benzo[d]oxazol-5- ylamino)quinolin-6-yl)-3- fluorophenyl)(morpholino) methanone |
| 366 | | 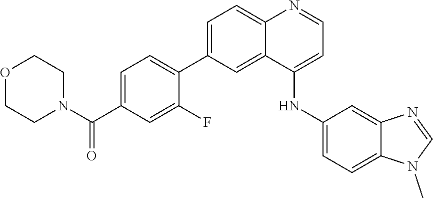 [SMILES: Cn1cnc2cc(Nc3ccnc4ccc(-c5ccc(C(=O)N6CCOCC6)cc5F)cc34)ccc21] | (3-fluoro-4-(4-((1-methyl- 1H-benzo[d]imidazol-5- yl)amino)quinolin-6- yl)phenyl)(morpholino) methanone |
| 367 | | 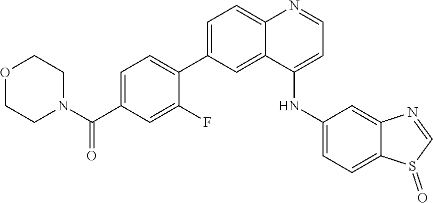 [SMILES: O=C(c1ccc(-c2ccc3nccc(Nc4ccc5c(c4)N=CS5=O)c3c2)c(F)c1)N1CCOCC1] | (3-fluoro-4-(4-((1- oxidobenzo[d]thiazol-5- yl)amino)quinolin-6- yl)phenyl)(morpholino) methanone |
| 368 | | 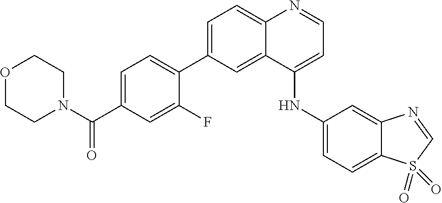 [SMILES: O=C(c1ccc(-c2ccc3nccc(Nc4ccc5c(c4)N=CS5(=O)=O)c3c2)c(F)c1)N1CCOCC1] | (4-(4-((1,1- dioxidobenzo[d]thiazol-5- yl)amino)quinolin-6-yl)-3- fluorophenyl)(morpholino) methanone |
| 369 (also referred to as Com- pound 372) | | 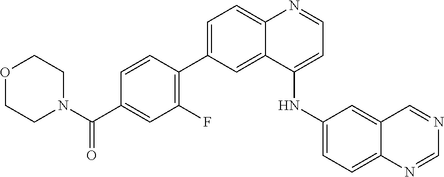 [SMILES: O=C(c1ccc(-c2ccc3nccc(Nc4ccc5ncncc5c4)c3c2)c(F)c1)N1CCOCC1] | (3-fluoro-4-(4-(quinazolin- 6-ylamino)quinolin-6- yl)phenyl)(morpholino) methanone |
| 370 | | 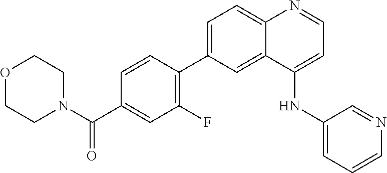 [SMILES: O=C(c1ccc(-c2ccc3nccc(Nc4cccnc4)c3c2)c(F)c1)N1CCOCC1] | (3-fluoro-4-(4-(pyridin-3- ylamino)quinolin-6- yl)phenyl)(morpholino) methanone |
| 371 | | 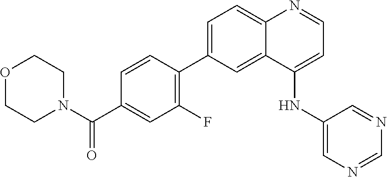 [SMILES: O=C(c1ccc(-c2ccc3nccc(Nc4cncnc4)c3c2)c(F)c1)N1CCOCC1] | (3-fluoro-4-(4-(pyrimidin- 5-ylamino)quinolin-6- yl)phenyl)(morpholino) methanone |
| 372 (also referred to as Com- pound 369) | | 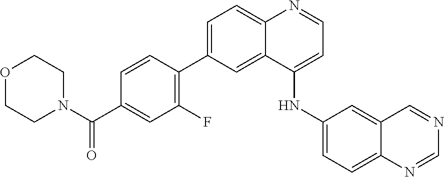 [SMILES: O=C(c1ccc(-c2ccc3nccc(Nc4ccc5ncncc5c4)c3c2)c(F)c1)N1CCOCC1] | (3-fluoro-4-(4-(quinazolin- 6-ylamino)quinolin-6- yl)phenyl)(morpholino) methanone |
| 373 (also referred to as Com- pound 231) | | 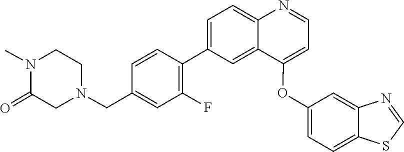 [SMILES: CN1CCN(Cc2ccc(-c3ccc4nccc(Oc5ccc6scnc6c5)c4c3)c(F)c2)CC1=O] | 4-(4-(4-(benzo[d]thiazol-5- yloxy)quinolin-6-yl)-3- fluorobenzyl)-1- methylpiperazin-2-one |
| 374 | | 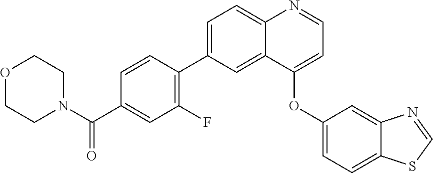 [SMILES: O=C(c1ccc(-c2ccc3nccc(Oc4ccc5scnc5c4)c3c2)c(F)c1)N1CCOCC1] | (4-(4-(benzo[d]thiazol-5- yloxy)quinolin-6-yl)-3- fluorophenyl)(morpholino) methanone |
| 375 | | 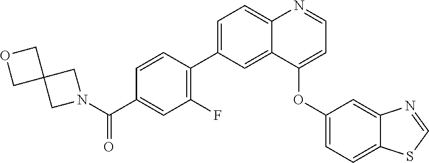 [SMILES: O=C(c1ccc(-c2ccc3nccc(Oc4ccc5scnc5c4)c3c2)c(F)c1)N1CC2(COC2)C1] | (4-(4-(benzo[d]thiazol-5- yloxy)quinolin-6-yl)-3- fluorophenyl)(2-oxa-6- azaspiro[3.3]heptan-6- yl)methanone |
| 376 | | 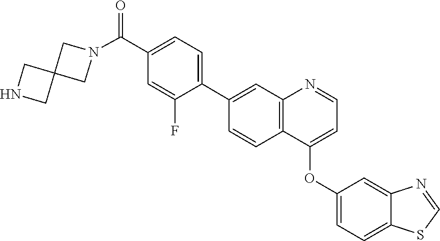 [SMILES: O=C(c1ccc(-c2ccc3c(Oc4ccc5scnc5c4)ccnc3c2)c(F)c1)N1CC2(CNC2)C1] | (4-(4-(benzo[d]thiazol-5- yloxy)quinolin-7-yl)-3- fluorophenyl)(2,6- diazaspiro[3.3]heptan-2- yl)methanone |
| 377 | | 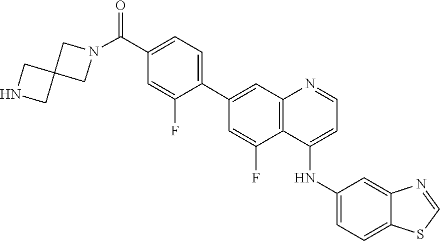 [SMILES: O=C(c1ccc(-c2cc(F)c3c(Nc4ccc5scnc5c4)ccnc3c2)c(F)c1)N1CC2(CNC2)C1] | (4-(4-(benzo[d]thiazol-5- ylamino)-5-fluoroquinolin- 7-yl)-3-fluorophenyl)(2,6- diazaspiro[3.3]heptan-2- yl)methanone |
| 378 | | 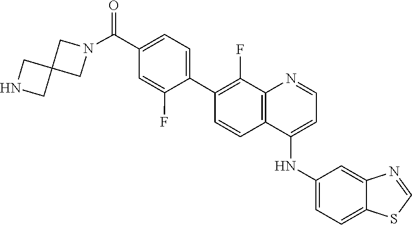 [SMILES: O=C(c1ccc(-c2ccc3c(Nc4ccc5scnc5c4)ccnc3c2F)c(F)c1)N1CC2(CNC2)C1] | (4-(4-(benzo[d]thiazol-5- ylamino)-8-fluoroquinolin- 7-yl)-3-fluorophenyl)(2,6- diazaspiro[3.3]heptan-2- yl)methanone |
| 379 | | 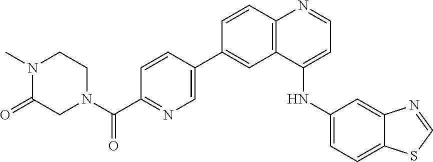 [SMILES: CN1CCN(C(=O)c2ccc(-c3ccc4nccc(Nc5ccc6scnc6c5)c4c3)cn2)CC1=O] | 4-(5-(4-(benzo[d]thiazol-5- ylamino)quinolin-6- yl)picolinoyl)-1- methylpiperazin-2-one |
| 380 | | 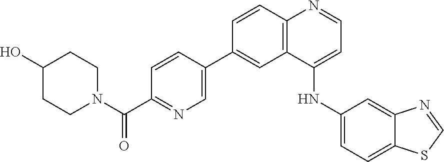 [SMILES: O=C(c1ccc(-c2ccc3nccc(Nc4ccc5scnc5c4)c3c2)cn1)N1CCC(O)CC1] | (5-(4-(benzo[d]thiazol-5- ylamino)quinolin-6- yl)pyridin-2-yl)(4- hydroxypiperidin-1- yl)methanone |
| 381 | | 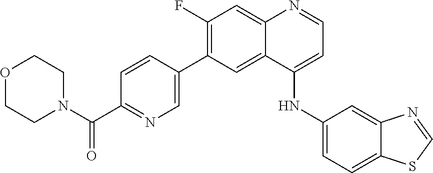 [SMILES: O=C(c1ccc(-c2cc3c(Nc4ccc5scnc5c4)ccnc3cc2F)cn1)N1CCOCC1] | (5-(4-(benzo[d]thiazol-5- ylamino)-7-fluoroquinolin- 6-yl)pyridin-2- yl)(morpholino)methanone |
| 382 | | 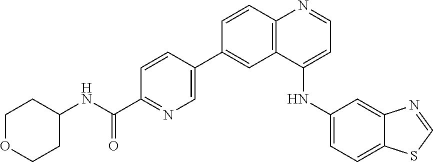 [SMILES: O=C(NC1CCOCC1)c1ccc(-c2ccc3nccc(Nc4ccc5scnc5c4)c3c2)cn1] | 5-(4-(benzo[d]thiazol-5- ylamino)quinolin-6-yl)-N- (tetrahydro-2H-pyran-4- yl)picolinamide |
| 383 | | 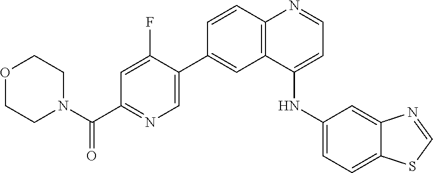 [SMILES: O=C(c1cc(F)c(-c2ccc3nccc(Nc4ccc5scnc5c4)c3c2)cn1)N1CCOCC1] | (5-(4-(benzo[d]thiazol-5- ylamino)quinolin-6-yl)-4- fluoropyridin-2- yl)(morpholino)methanone |
| 384 | | 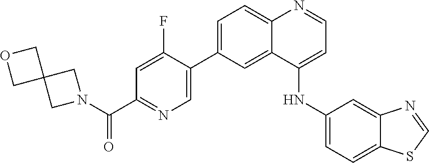 [SMILES: O=C(c1cc(F)c(-c2ccc3nccc(Nc4ccc5scnc5c4)c3c2)cn1)N1CC2(COC2)C1] | (5-(4-(benzo[d]thiazol-5- ylamino)quinolin-6-yl)-4- fluoropyridin-2-yl)(2-oxa- 6-azaspiro[3.3]heptan-6- yl)methanone |
| 385 (also referred to as Com- pound 255) | 78 | 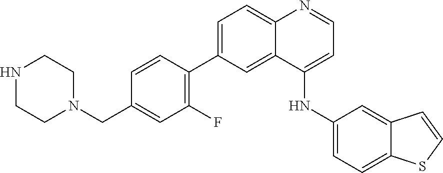 [SMILES: Fc1cc(CN2CCNCC2)ccc1-c1ccc2nccc(Nc3ccc4sccc4c3)c2c1] | N-(benzo[b]thiophen-5-yl)- 6-(2-fluoro-4-(piperazin-1- ylmethyl)phenyl)quinolin- 4-amine |
| 386 (also referred to as Com- pound 264) | 79 | 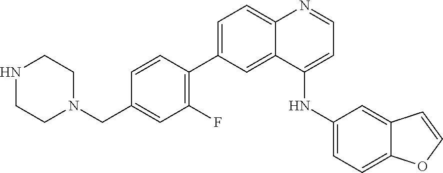 [SMILES: Fc1cc(CN2CCNCC2)ccc1-c1ccc2nccc(Nc3ccc4occc4c3)c2c1] | N-(benzofuran-5-yl)-6-(2- fluoro-4-(piperazin-1- ylmethyl)phenyl)quinolin- 4-amine |
| 387 (also referred to as Com- pound 270) | 80 | 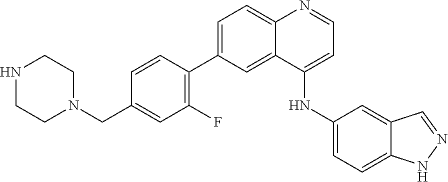 [SMILES: Fc1cc(CN2CCNCC2)ccc1-c1ccc2nccc(Nc3ccc4[nH]ncc4c3)c2c1] | 6-(2-fluoro-4-(piperazin-1- ylmethyl)phenyl)-N-(1H- indazol-5-yl)quinolin-4- amine |
| 388 (also referred to as Com- pound 267) | 81 | 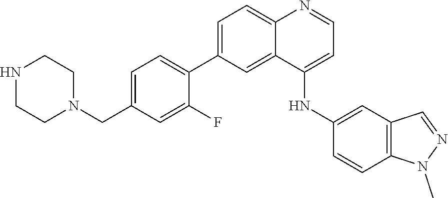 [SMILES: Cn1ncc2cc(Nc3ccnc4ccc(-c5ccc(CN6CCNCC6)cc5F)cc34)ccc21] | 6-(2-fluoro-4-(piperazin-1- ylmethyl)phenyl)-N-(1- methyl-1H-indazol-5- yl)quinolin-4-amine |
| 389 | | 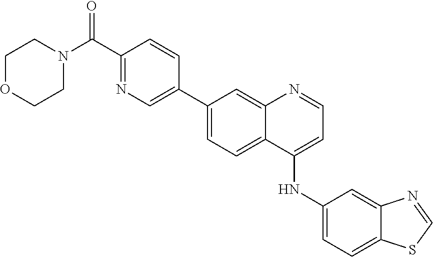 [SMILES: O=C(c1ccc(-c2ccc3c(Nc4ccc5scnc5c4)ccnc3c2)cn1)N1CCOCC1] | (5-(4-(benzo[d]thiazol-5- ylamino)quinolin-7- yl)pyridin-2- yl)(morpholino)methanone |
| 390 | | 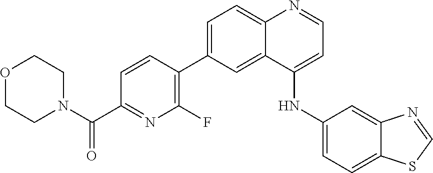 [SMILES: O=C(c1ccc(-c2ccc3nccc(Nc4ccc5scnc5c4)c3c2)c(F)n1)N1CCOCC1] | (5-(4-(benzo[d]thiazol-5- ylamino)quinolin-6-yl)-6- fluoropyridin-2- yl)(morpholino)methanone |
| 391 | | 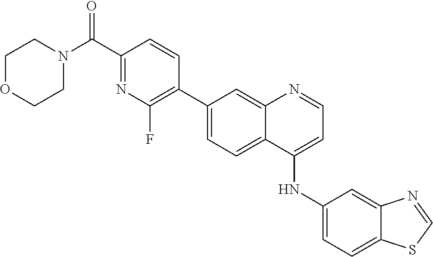 [SMILES: O=C(c1ccc(-c2ccc3c(Nc4ccc5scnc5c4)ccnc3c2)c(F)n1)N1CCOCC1] | (5-(4-(benzo[d]thiazol-5- ylamino)quinolin-7-yl)-6- fluoropyridin-2- yl)(morpholino)methanone |
| 392 | | 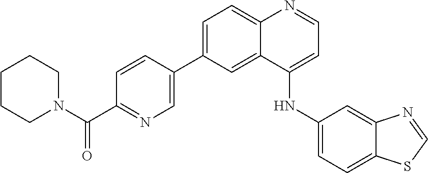 [SMILES: O=C(c1ccc(-c2ccc3nccc(Nc4ccc5scnc5c4)c3c2)cn1)N1CCCCC1] | (5-(4-(benzo[d]thiazol-5- ylamino)quinolin-6- yl)pyridin-2-yl)(piperidin- 1-yl)methanone |
| 393 | | 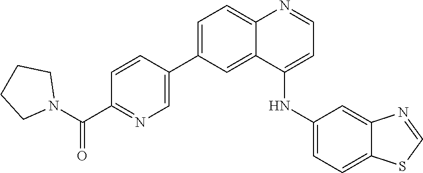 [SMILES: O=C(c1ccc(-c2ccc3nccc(Nc4ccc5scnc5c4)c3c2)cn1)N1CCCC1] | (5-(4-(benzo[d]thiazol-5- ylamino)quinolin-6- yl)pyridin-2-yl)(pyrrolidin- 1-yl)methanone |
| 394 | | 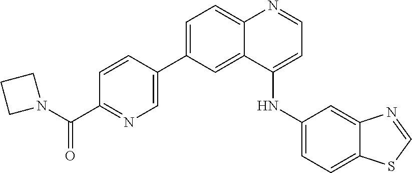 [SMILES: O=C(c1ccc(-c2ccc3nccc(Nc4ccc5scnc5c4)c3c2)cn1)N1CCC1] | azetidin-1-yl(5-(4- (benzo[d]thiazol-5- ylamino)quinolin-6- yl)pyridin-2-yl)methanone |
| 395 | | 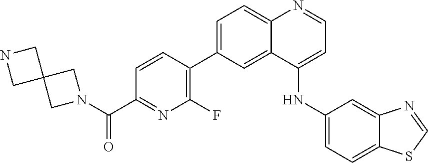 [SMILES: O=C(c1ccc(-c2ccc3nccc(Nc4ccc5scnc5c4)c3c2)c(F)n1)N1CC2(CNC2)C1] | (5-(4-(benzo[d]thiazol-5- ylamino)quinolin-6-yl)-6- fluoropyridin-2-yl)(2,6- diazaspiro[3.3]heptan-2- yl)methanone |
| 396 | | 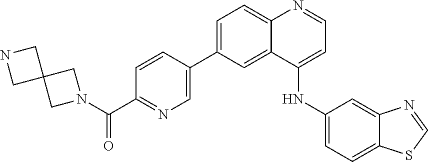 [SMILES: O=C(c1ccc(-c2ccc3nccc(Nc4ccc5scnc5c4)c3c2)cn1)N1CC2(CNC2)C1] | N-(6-(6-((2,6- diazaspiro[3.3]heptan-2- yl)methyl)pyridin-3- yl)quinolin-4- yl)benzo[d]thiazol-5-amine |
| 397 | | 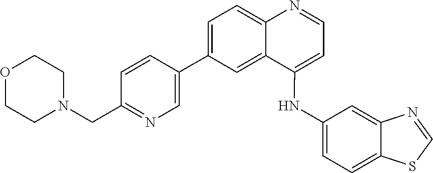 [SMILES: c1cc(Nc2ccc3scnc3c2)c2cc(-c3ccc(CN4CCOCC4)nc3)ccc2n1] | N-(6-(6- (morpholinomethyl)pyridin- 3-yl)quinolin-4- yl)benzo[d]thiazol-5-amine |
| 398 | | 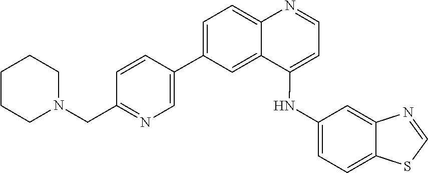 [SMILES: c1cc(Nc2ccc3scnc3c2)c2cc(-c3ccc(CN4CCCCC4)nc3)ccc2n1] | N-(6-(6-(piperidin-1- ylmethyl)pyridin-3- yl)quinolin-4- yl)benzo[d]thiazol-5-amine |
| 399 | | 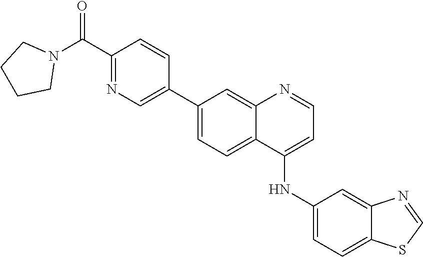 [SMILES: O=C(c1ccc(-c2ccc3c(Nc4ccc5scnc5c4)ccnc3c2)cn1)N1CCCC1] | (5-(4-(benzo[d]thiazol-5- ylamino)quinolin-7- yl)pyridin-2-yl)(pyrrolidin- 1-yl)methanone |
| 400 | | 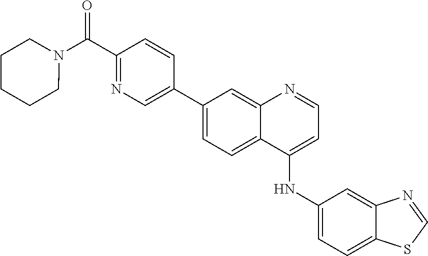 [SMILES: O=C(c1ccc(-c2ccc3c(Nc4ccc5scnc5c4)ccnc3c2)cn1)N1CCCCC1] | (5-(4-(benzo[d]thiazol-5- ylamino)quinolin-7- yl)pyridin-2-yl)(piperidin- 1-yl)methanone |
| 401 | | 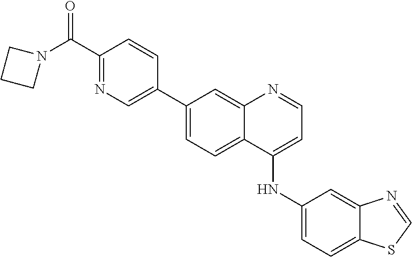 [SMILES: O=C(c1ccc(-c2ccc3c(Nc4ccc5scnc5c4)ccnc3c2)cn1)N1CCC1] | azetidin-1-yl(5-(4- (benzo[d]thiazol-5- ylamino)quinolin-7- yl)pyridin-2-yl)methanone |
| 402 | | 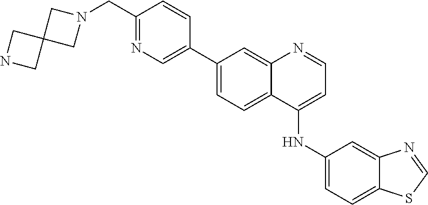 [SMILES: c1cc(Nc2ccc3scnc3c2)c2ccc(-c3ccc(CN4CC5(CNC5)C4)nc3)cc2n1] | N-(7-(6-((2,6- diazaspiro[3.3]heptan-2- yl)methyl)pyridin-3- yl)quinolin-4- yl)benzo[d]thiazol-5-amine |
| 403 | | 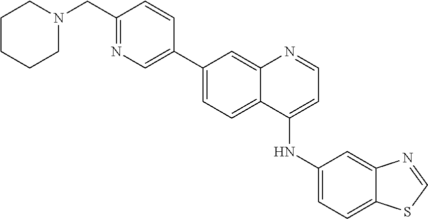 [SMILES: c1cc(Nc2ccc3scnc3c2)c2ccc(-c3ccc(CN4CCCCC4)nc3)cc2n1] | N-(7-(6-(piperidin-1- ylmethyl)pyridin-3- yl)quinolin-4- yl)benzo[d]thiazol-5-amine |
| 404 | | 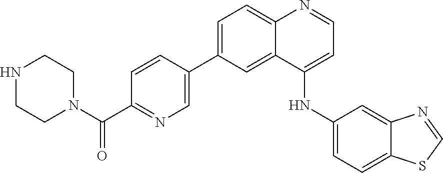 [SMILES: O=C(c1ccc(-c2ccc3nccc(Nc4ccc5scnc5c4)c3c2)cn1)N1CCNCC1] | (5-(4-(benzo[d]thiazol-5- ylamino)quinolin-6- yl)pyridin-2-yl)(piperazin- 1-yl)methanone |

Formula (Ia) is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Table 1 has two or more stereocenters, also provided are any enantiomer or diastereomer of the compound. For example, if a compound of Table 1 contains a first stereocenter and a second stereocenter with “R” and “R” stereochemical configurations, respectively, also provided are stereoisomers of the compound having first and second stereocenters with “S” and “S” stereochemical configurations, respectively, “S” and “R” stereochemical configurations, respectively, and “R” and “S” stereochemical configurations, respectively. If a compound of Table 1 contains a first stereocenter and a second stereocenter with “S” and “S” stereochemical configurations, respectively, also provided are stereoisomers of the compound having first and second stereocenters with “R” and “R” stereochemical configurations, respectively, “S” and “R” stereochemical configurations, respectively, and “R” and “S” stereochemical configurations, respectively. If a compound of Table 1 contains a first stereocenter and a second stereocenter with “S” and “R” stereochemical configurations, respectively, also provided are stereoisomers of the compound having first and second stereocenters with “R” and “S” stereochemical configurations, respectively, “R” and “R” stereochemical configurations, respectively, and “S” and “S” stereochemical configurations, respectively. Similarly, if a compound of Table 1 contains a first stereocenter and a second stereocenter with “R” and “S” stereochemical configurations, respectively, also provided are stereoisomers of the compound having first and second stereocenters with “S” and “R” stereochemical configurations, respectively, “R” and “R” stereochemical configurations, respectively, and “S” and “S” stereochemical configurations, respectively. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

The compound names provided herein, including in Table 1, are provided by ChemBioDraw Professional. One of skill in the art would understand that the compounds may be named or identified using various commonly recognized nomenclature systems and symbols. By way of example, the compounds may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

### III. Pharmaceutical Compositions

In some embodiments, the compounds provided herein can be formulated in a composition, such as a pharmaceutical composition. In some embodiments, the composition further comprises one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium croscarmellose, glucose, gelatin, sucrose, and magnesium carbonate. In some embodiments, provided are compositions, such as pharmaceutical compositions that contain one or more compounds described herein, or a pharmaceutically acceptable salt, stereoisomer, or tautomer thereof, or a pharmaceutically acceptable salt thereof.

Also provided are packaged pharmaceutical compositions, comprising a pharmaceutical composition as described herein and instructions for using the composition to treat a patient suffering from a disease or condition described herein.

### IV. Method of Use

In yet another aspect, provided herein is a method of inhibiting RIPK3 comprising contacting the kinase with a compound described herein (e.g., a compound of Formula (Ia), (I), (II-1), (II-2), (III-1), (III-2), (IV-1), or (IV-2)), or a pharmaceutically acceptable salt, stereoisomer, or tautomer thereof, or the pharmaceutical composition described herein.

In another aspect, provided herein is a method of treating a disease or condition mediated by inhibition of RIPK3 in an individual in need thereof comprising administering to the individual a compound described herein (e.g., a compound of Formula (Ia), (I), (II-1), (II-2), (III-1), (III-2), (IV-1), or (IV-2)), or a pharmaceutically acceptable salt, stereoisomer, or tautomer thereof, or the pharmaceutical composition described herein. In some embodiments, the disease or condition mediated by the inhibition of RIPK3 is a systemic inflammatory response, an autoimmune disease, a proliferative disorder, a metabolic disease, a neurodegenerative disease or pain. In some embodiments, the disease or disorder is an age-related neurodegenerative disease or condition. In some embodiments, the disease or disorder is sepsis. In some embodiments, the disease or disorder is pain.

### V. Dose and Method of Administration

In some variations, the compounds and compositions described herein are administered at a therapeutically effective dosage, e.g., a dosage sufficient to provide treatment for the disease state. The amount of the chemical entity administered will be dependent, for example, on the subject (e.g., human) and disease state being treated, the severity of the affliction, the manner and schedule of administration and the judgment of the prescribing physician.

Administration of the compounds and compositions described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport patch forms) for prolonged timed, and/or pulsed administration at a predetermined rate. In some embodiments, the compositions are provided in unit dosage forms suitable for single administration of a precise dose.

The compounds disclosed and/or described herein can be administered either alone or in combination with one or more conventional pharmaceutical carriers or excipients (e.g., mannitol, lactose, starch, magnesium stearate, sodium saccharine, talcum, cellulose, sodium croscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Actual methods of preparing such dosage forms are known, or will be apparent, to those skilled in this art; for example, see Remington's Pharmaceutical Sciences, Mack Publishing Company, Easton, Pennsylvania.

### VI. Kits

Also provided herein are kits for carrying out the methods described herein, which comprises one or more compounds described herein, or a pharmaceutically acceptable salt, stereoisomer, or tautomer thereof, or a pharmaceutically acceptable salt thereof, or a pharmaceutical composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use in the treatment of a disease mediated by inhibition of RIPK3.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disease detailed herein (e.g., a disease mediated by inhibition of RIPK3) to provide effective treatment of an individual for an extended period. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods described herein. The instructions included with the kit generally include information as to the components and their administration to an individual.

### ENUMERATED EMBODIMENTS

The following enumerated embodiments are representative of some aspects of the invention.

Enumerated Embodiment 1. A compound of formula (I):

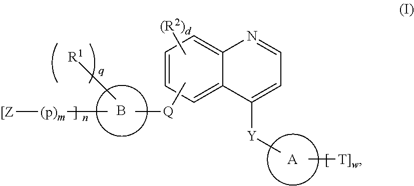

or a pharmaceutically acceptable salt, stereoisomer, or tautomer thereof, wherein:
d is an integer from 0 to 3;
m is an integer from 0 to 2;
n is an integer from 0 to 5;
q is an integer from 0 to 4;
w is an integer from 0 to 9;
Q is a bond or —NH—;
Ring B is C₆₋₁₄ aryl or pyridyl;
each R¹ is independently halo, —CN, C₁₋₆ alkyl or C₁₋₆ alkoxy;
each R² is independently halo;
each p is independently selected from the group consisting of —O—, C₁alkylene, —NR^{a}—, —C(O)—, #—NR^{a}SO₂—, #—R^{a}OR^{b}—, #—C(O)NR^{a}R^{b}—, #—C(O)R^{a}—, #—C(O)OR^{a}, and #—OR^{a}—, wherein #indicates the point of attachment to Ring B;
each of R^{a} and R^{b} is independently H, C₁₋₆ alkyl or C₁₋₆ alkylene;
each Z is independently —NR^{c}R^{d}, 3- to 12-membered heterocyclyl optionally substituted with one or more R^{Za}, 5- to 12-membered heteroaryl optionally substituted with one or more R^{Zb}, or C₆₋₁₄ aryl optionally substituted with one or more R^{Zc}.
each R^{Za}, R^{Zb}, and R^{Zc} is independently selected from the group consisting of oxo, halo, —OH, O⁻, —NR^{a}R^{b}, C₁₋₆ alkyl, C₁₋₆ alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, and R^{Zc} is optionally substituted with one or more halo, —OH, C₁₋₆ alkyl, —C(O)OR^{c}, or —C(O)NR^{c}R^{d};
each R^{c} and R^{d} is independently H or C₁₋₆ alkyl optionally substituted with one or more —OH, halo, —NH₂, —CF₃, or oxo;
Y is —NH— or —O—;
Ring A is selected from the group consisting of:

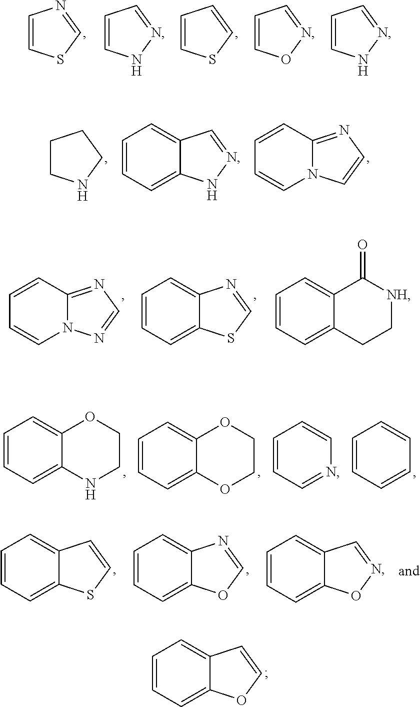

and
each T is independently selected from the group consisting of halo, —NH₂, C₁-C₆ alkyl, C₁₋₆ alkoxy, —NHC(O)—C₃₋₆cycloalkyl, and methylpiperidyl, provided that:
   (1) n+w is an integer greater than 0, and
   (2) when Ring A is phenyl, then either: (a) n is an integer from 1 to 5 and Ring B is substituted by at least one (p)ₘ-Z wherein Z is 3- to 12-membered heterocyclyl substituted with one or more R^{Za} or (b) Ring A is substituted by at least one T wherein T is methylpiperidyl.
      Enumerated Embodiment 2. The compound of Enumerated Embodiment 1, or a pharmaceutically acceptable salt, stereoisomer, or tautomer thereof, wherein Q is —NH—.
      Enumerated Embodiment 3. The compound of Enumerated Embodiment 1, or a pharmaceutically acceptable salt, stereoisomer, or tautomer thereof, wherein Q is a bond.
      Enumerated Embodiment 4. The compound of any one of Enumerated Embodiments 1-3, or a pharmaceutically acceptable salt, stereoisomer, or tautomer thereof, wherein Ring B is C₆₋₁₄ aryl.
      Enumerated Embodiment 5. The compound of Enumerated Embodiment 4, wherein Ring B is phenyl.
      Enumerated Embodiment 6. The compound of any one of Enumerated Embodiments 1-3, or a pharmaceutically acceptable salt, stereoisomer, or tautomer thereof, wherein Ring B is pyridyl.
      Enumerated Embodiment 7. The compound of any one of Enumerated Embodiments 1-6, or a pharmaceutically acceptable salt, stereoisomer, or tautomer thereof, wherein q is an integer from 1-4 and at least one R¹ is halo.
      Enumerated Embodiment 8. The compound of Enumerated Embodiment 7, or a pharmaceutically acceptable salt, stereoisomer, or tautomer thereof, wherein R¹ is F.
      Enumerated Embodiment 9. The compound of Enumerated Embodiment 7, or a pharmaceutically acceptable salt, stereoisomer, or tautomer thereof, wherein R¹ is Cl.
      Enumerated Embodiment 10. The compound of any one of Enumerated Embodiments 1-9, or a pharmaceutically acceptable salt, stereoisomer, or tautomer thereof, wherein q is an integer from 1-4 and at least one R¹ is —CN.
      Enumerated Embodiment 11. The compound of any one of Enumerated Embodiments 1-10, or a pharmaceutically acceptable salt, stereoisomer, or tautomer thereof, wherein q is an integer from 1-4 and at least one R¹ is C₁₋₆ alkyl.
      Enumerated Embodiment 12. The compound of any one of Enumerated Embodiments 1-11, or a pharmaceutically acceptable salt, stereoisomer, or tautomer thereof, wherein q is an integer from 1-4 and at least one R¹ is C₁₋₆ alkoxy.
      Enumerated Embodiment 13. The compound of any one of Enumerated Embodiments 1-12, or a pharmaceutically acceptable salt, stereoisomer, or tautomer thereof, wherein d is an integer from 1-3.
      Enumerated Embodiment 14. The compound of Enumerated Embodiment 13, or a pharmaceutically acceptable salt, stereoisomer, or tautomer thereof, wherein at least one R² is F.
      Enumerated Embodiment 15. The compound of Enumerated Embodiment 13, or a pharmaceutically acceptable salt, stereoisomer, or tautomer thereof, wherein at least one R² is C₁.
      Enumerated Embodiment 16. The compound of any one of Enumerated Embodiments 1-15, or a pharmaceutically acceptable salt, stereoisomer, or tautomer thereof, wherein Y is —NH—.
      Enumerated Embodiment 17. The compound of any one of Enumerated Embodiments 1-15, or a pharmaceutically acceptable salt, stereoisomer, or tautomer thereof, wherein Y is —O—.
      Enumerated Embodiment 18. The compound of any one of Enumerated Embodiments 1-17, or a pharmaceutically acceptable salt, stereoisomer, or tautomer thereof, wherein n is an integer from 1-5 and at least one Z is —NR^{c}R^{d}.
      Enumerated Embodiment 19. The compound of any one of Enumerated Embodiments 1-17, or a pharmaceutically acceptable salt, stereoisomer, or tautomer thereof, wherein n is an integer from 1-5 and at least one Z is 3- to 12-membered heterocyclyl optionally substituted with one or more R^{Za}.
      Enumerated Embodiment 20. The compound of Enumerated Embodiment 19, or a pharmaceutically acceptable salt, stereoisomer, or tautomer thereof, wherein Z is substituted with one or more R^{Za} that is C₁₋₆alkyl.
      Enumerated Embodiment 21. The compound of Enumerated Embodiment 19, or a pharmaceutically acceptable salt, stereoisomer, or tautomer thereof, wherein Z is substituted with one or more R^{Za} that is oxo.
      Enumerated Embodiment 22. The compound of any one of Enumerated Embodiments 1-17, or a pharmaceutically acceptable salt, stereoisomer, or tautomer thereof, wherein n is an integer from 1-5 and Z is 5- to 12-membered heteroaryl optionally substituted with one or more R^{Zb}.
      Enumerated Embodiment 23. The compound of any one of Enumerated Embodiments 1-17, or a pharmaceutically acceptable salt, stereoisomer, or tautomer thereof, wherein n is an integer from 1-5 and Z is C₆₋₁₄ aryl, optionally substituted with one or more R^{Zc}.
      Enumerated Embodiment 24. The compound of Enumerated Embodiment 23, or a pharmaceutically acceptable salt, stereoisomer, or tautomer thereof, wherein Z is substituted with one or more R^{Zc} that is halo.
      Enumerated Embodiment 25. The compound of any one of Enumerated Embodiments 1-24, wherein n is 1.
      Enumerated Embodiment 26. The compound of any one of Enumerated Embodiments 1-25, or a pharmaceutically acceptable salt, stereoisomer, or tautomer thereof, wherein Ring A is

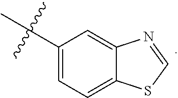 [SMILES: *c1ccc2scnc2c1]

Enumerated Embodiment 27. The compound any one of Enumerated Embodiments 1-25, or a pharmaceutically acceptable salt, stereoisomer, or tautomer thereof, wherein Ring A is

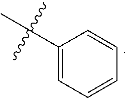 [SMILES: *c1ccccc1]

Enumerated Embodiment 28. The compound any one of Enumerated Embodiments 1-25, or a pharmaceutically acceptable salt, stereoisomer, or tautomer thereof, wherein Ring A is

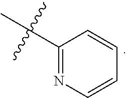 [SMILES: *c1ccccn1]

Enumerated Embodiment 29. The compound of any one of Enumerated Embodiments 1-28, or a pharmaceutically acceptable salt, stereoisomer, or tautomer thereof, wherein w is an integer from 1-5 and at least one T is halo.
Enumerated Embodiment 30. The compound of Enumerated Embodiment 29, or a pharmaceutically acceptable salt, stereoisomer, or tautomer thereof, wherein at least one T is F.
Enumerated Embodiment 31. The compound of Enumerated Embodiment 29, or a pharmaceutically acceptable salt, stereoisomer, or tautomer thereof, wherein at least one T is C₁.
Enumerated Embodiment 32. The compound of any one of Enumerated Embodiments 1-31, or a pharmaceutically acceptable salt, stereoisomer, or tautomer thereof, wherein w is an integer from 1-5 and at least one T is —NH₂.
Enumerated Embodiment 33. The compound of any one of Enumerated Embodiments 1-32, or a pharmaceutically acceptable salt, stereoisomer, or tautomer thereof, wherein w is an integer from 1-5 and at least one T is C₁-C₆ alkyl.
Enumerated Embodiment 34. The compound of any one of Enumerated Embodiments 1-33, or a pharmaceutically acceptable salt, stereoisomer, or tautomer thereof, wherein w is an integer from 1-5 and at least one T is NHC(O)—C₃₋₆cycloalkyl.
Enumerated Embodiment 35. The compound of any one of Enumerated Embodiments 1-34, or a pharmaceutically acceptable salt, stereoisomer, or tautomer thereof, wherein w is an integer from 1-5 and at least one T is methylpiperidyl.
Enumerated Embodiment 36. The compound of any one of Enumerated Embodiments 1-35, or a pharmaceutically acceptable salt, stereoisomer, or tautomer thereof, wherein w is an integer from 1-5 and at least one T is methoxy.
Enumerated Embodiment 37. The compound of any one of Enumerated Embodiments 1-36, or a pharmaceutically acceptable salt, stereoisomer, or tautomer thereof, wherein w is an integer from 1-3.
Enumerated Embodiment 38. The compound of any one of Enumerated Embodiments 1-28, or a pharmaceutically acceptable salt, stereoisomer, or tautomer thereof, wherein w is 0.
Enumerated Embodiment 39. The compound of any one of Enumerated Embodiments 1-37, or a pharmaceutically acceptable salt, stereoisomer, or tautomer thereof, wherein T is selected from the group consisting of —Cl, —F, —NH₂, —CH₃, wherein, for each T, * denotes the point of attachment to Ring A.

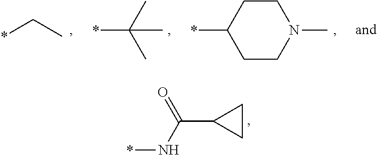

Enumerated Embodiment 40. The compound of any one of Enumerated Embodiments 1-39, or a pharmaceutically acceptable salt, stereoisomer, or tautomer thereof, wherein Z is selected from the group consisting of

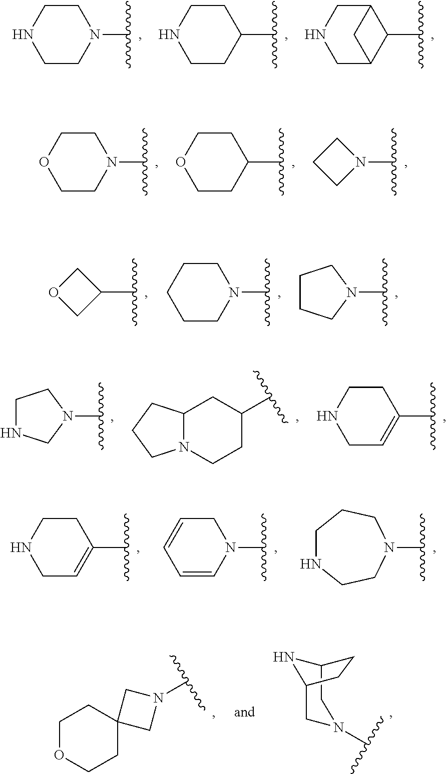

and each of which is optionally substituted with one or more R^{Za}

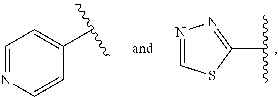 [SMILES: *c1ccncc1 ; *c1nncs1]

each of which is optionally substituted with one or more R^{Zb}; and

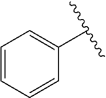 [SMILES: *c1ccccc1]

optionally substituted with one or more R^{Zc}.
Enumerated Embodiment 41. The compound of any one of Enumerated Embodiments 1-40, or a pharmaceutically acceptable salt, stereoisomer, or tautomer thereof, wherein R^{Za}, R^{Zb} or R^{Zc} is selected from the group consisting of ═O, —O—, —Cl, —F, —OH, —CH₃, —CF₃, NH₂,

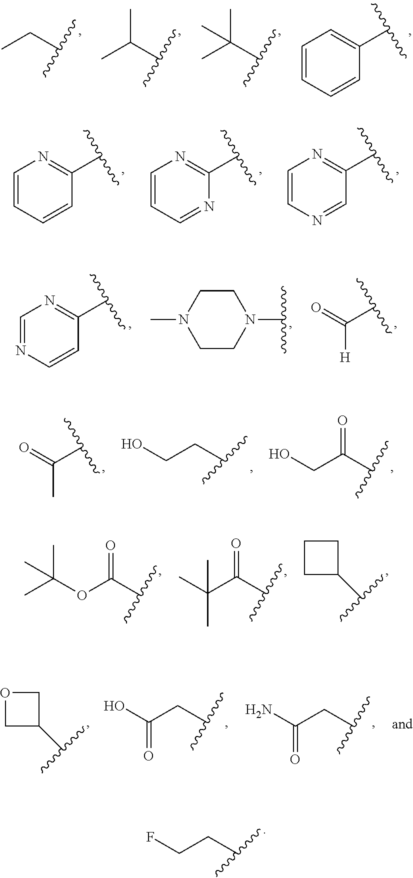

Enumerated Embodiment 42. The compound of any one of Enumerated Embodiments 1-41, or a pharmaceutically acceptable salt, stereoisomer, or tautomer thereof, wherein [Z-(p)ₘ] is selected from the group consisting of

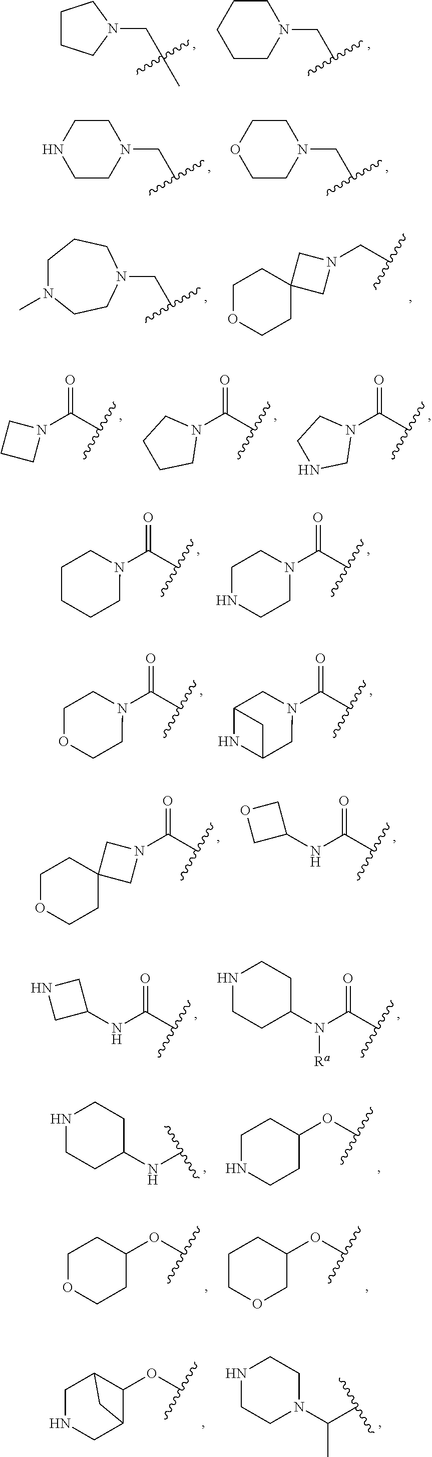

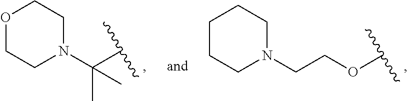

each of which is optionally substituted with one or more R^{Za};

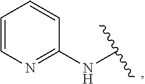 [SMILES: *Nc1ccccn1]

each of which is optionally substituted with one or more R_{Zb};

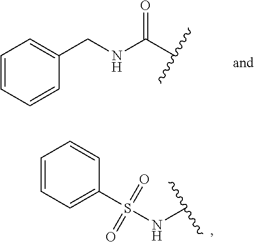 [SMILES: *C(=O)NCc1ccccc1 ; *NS(=O)(=O)c1ccccc1]

each of which is optionally substituted with one or more R^{Zc};

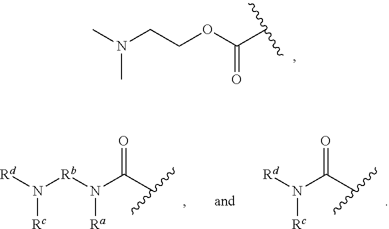

Enumerated Embodiment 43. The compound of Enumerated Embodiment 1, or a pharmaceutically acceptable salt, stereoisomer, or tautomer thereof, wherein the compound is of the formula (II-1):

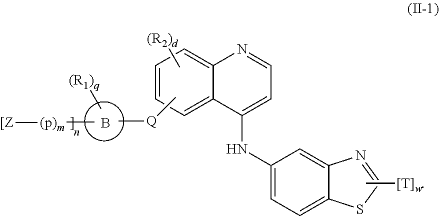

Enumerated Embodiment 44. The compound of Enumerated Embodiment 1, or a pharmaceutically acceptable salt, stereoisomer, or tautomer thereof, wherein the compound is of the formula (II-2):

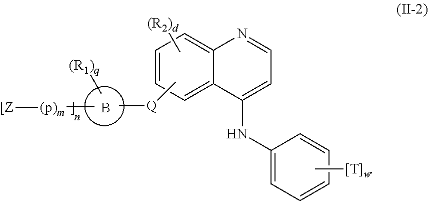

Enumerated Embodiment 45. The compound of Enumerated Embodiment 1, or a pharmaceutically acceptable salt, stereoisomer, or tautomer thereof, wherein the compound is of the formula (III-1):

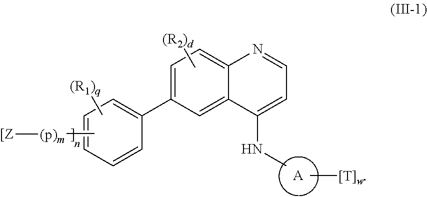

Enumerated Embodiment 46. The compound of Enumerated Embodiment 1, or a pharmaceutically acceptable salt, stereoisomer, or tautomer thereof, wherein the compound is of the formula (III-2):

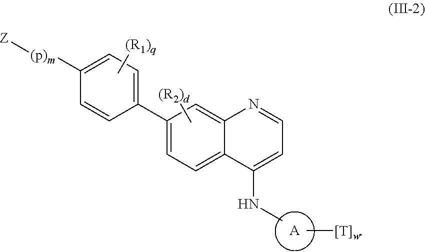

Enumerated Embodiment 47. The compound of Enumerated Embodiment 1, or a pharmaceutically acceptable salt, stereoisomer, or tautomer thereof, wherein the compound is of the formula (IV-1):

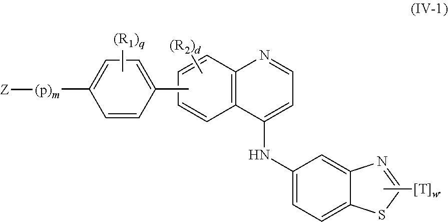

Enumerated Embodiment 48. The compound of Enumerated Embodiment 1, or a pharmaceutically acceptable salt, stereoisomer, or tautomer thereof, wherein the compound is of the formula (IV-2):

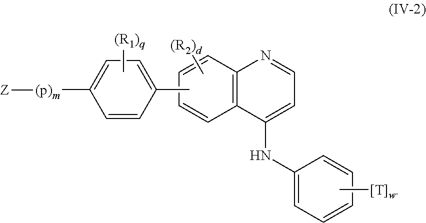

Enumerated Embodiment 49. The compound of Enumerated Embodiment 1, or a pharmaceutically acceptable salt, stereoisomer, or tautomer thereof, wherein the compound is a compound of Table 1.
Enumerated Embodiment 50. A pharmaceutical composition comprising the compound of any one of Enumerated Embodiments 1-49, or a pharmaceutically acceptable salt, stereoisomer, or tautomer thereof, and a pharmaceutically acceptable carrier.
Enumerated Embodiment 51. A method of inhibiting RIPK3 comprising contacting the kinase with a compound of any of one Enumerated Embodiments 1-49 or a pharmaceutically acceptable salt, stereoisomer, or tautomer thereof.
Enumerated Embodiment 52. A method of treating a disease or condition mediated by inhibition of RIPK3 in an individual in need thereof comprising administering to the individual a compound of any one of Enumerated Embodiments 1-49, or a pharmaceutically acceptable salt, stereoisomer, or tautomer thereof.
Enumerated Embodiment 53. The method of Enumerated Embodiment 52, wherein the disease or condition mediated by the inhibition of RIPK3 is a disease or condition is a systematic inflammatory response, an autoimmune disease, a proliferative disorder, a metabolic disease, a neurodegenerative disease or pain.
Enumerated Embodiment 54. The method of Enumerated Embodiment 53, wherein the disease or condition is an age-related neurodegenerative disease or condition.
Enumerated Embodiment 55. A pharmaceutical composition for the treatment of a disease mediated by inhibition of RIPK3, comprising the compound of any one of Enumerated Embodiments 1-49, or a pharmaceutically acceptable salt, stereoisomer, or tautomer thereof.
Enumerated Embodiment 56. A compound of any one of Enumerated Embodiments 1-49, or a pharmaceutically acceptable salt, stereoisomer, or tautomer thereof, for use in the treatment of a disease or condition mediated by inhibition of RIPK3.
Enumerated Embodiment 57. Use of a compound of any one of Enumerated Embodiments 1-49, or a pharmaceutically acceptable salt, stereoisomer, or tautomer thereof, in the manufacture of a medicament for use in the treatment of a disease mediated by inhibition of RIPK3.

### GENERAL SYNTHETIC EXAMPLES

The compounds of the invention may be prepared by a number of processes as generally described below and more specifically in the Examples hereinafter (such as the schemes provides in the Examples below). The chemical reactions described can be readily adapted to prepare a number of compounds as detailed herein. For example, the synthesis of non-exemplified compounds according to the invention can be performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds detailed herein.

Compounds provided herein may be prepared according to General Schemes 1, 2, 3, and/or 4.

In some embodiments, a compound of Formula (Ia) described herein can be synthesized following General Scheme 1.

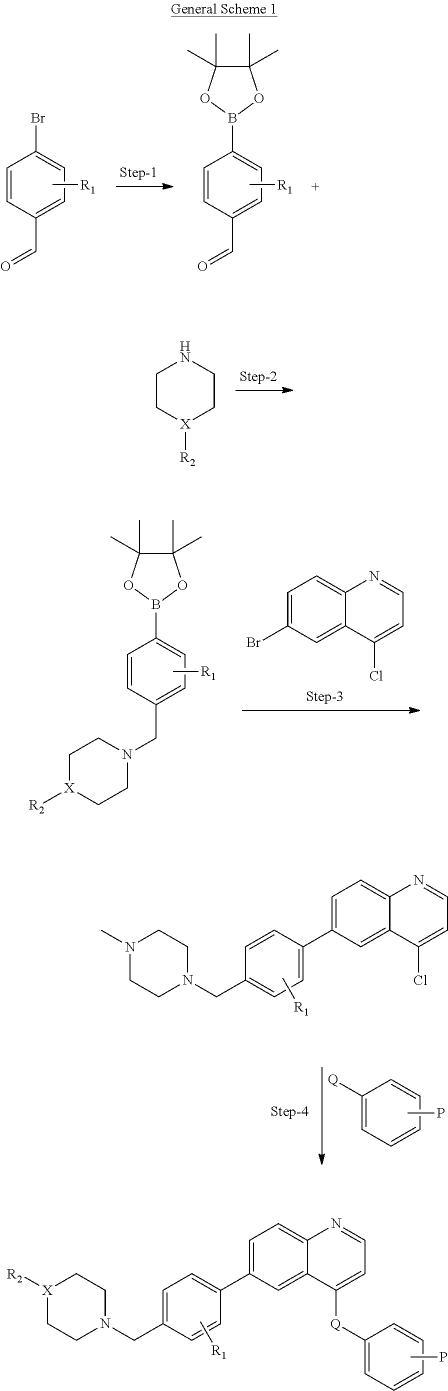

In some embodiments, a compound of Formula (Ia) described herein can be synthesized following General Scheme 2.

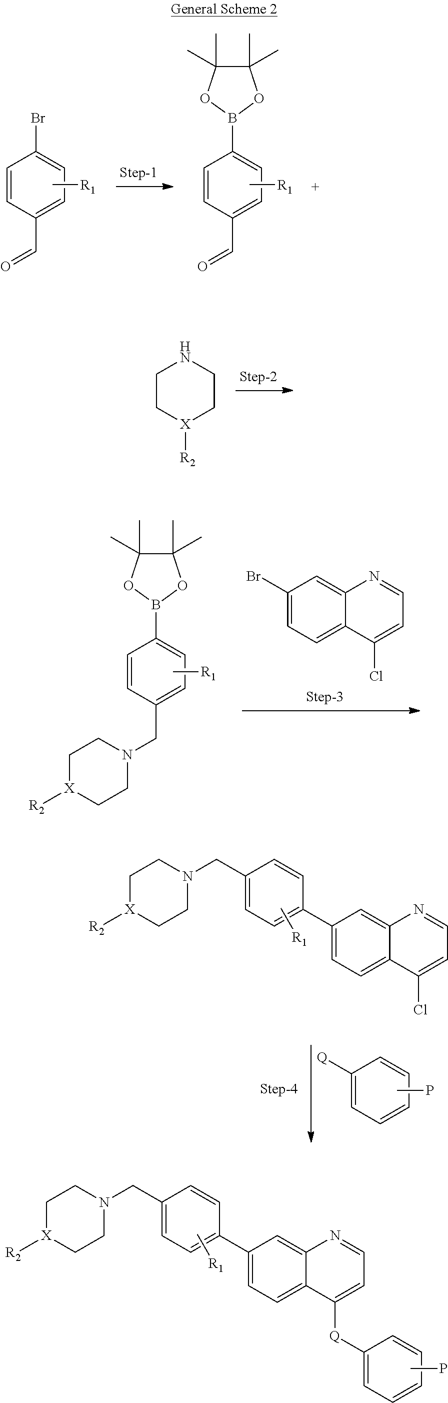

In some embodiments, a compound of Formula (Ia) described herein can be synthesized following General Scheme 3.

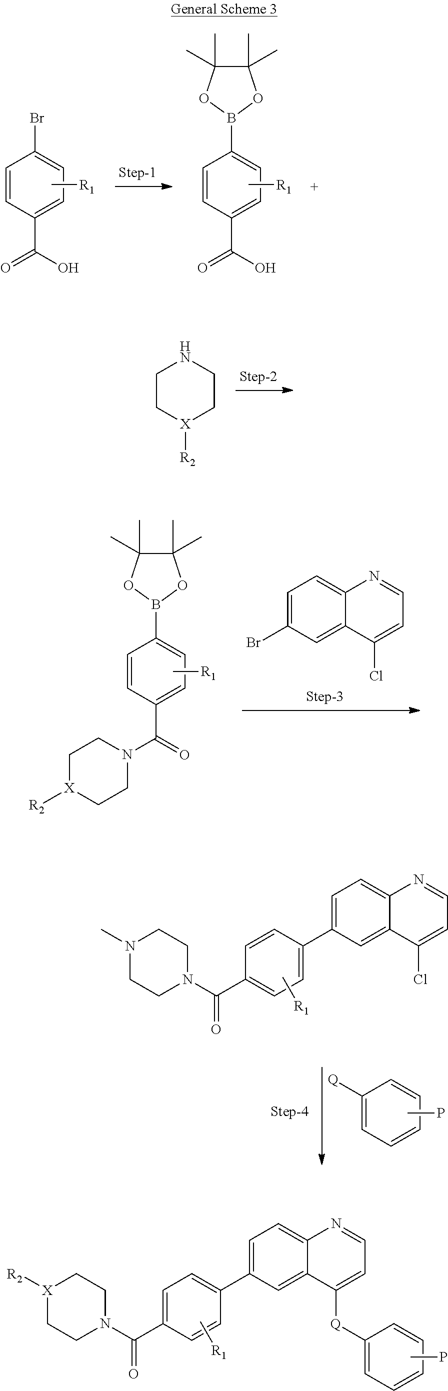

In some embodiments, a compound of Formula (Ia) described herein can be synthesized following General Scheme 4.

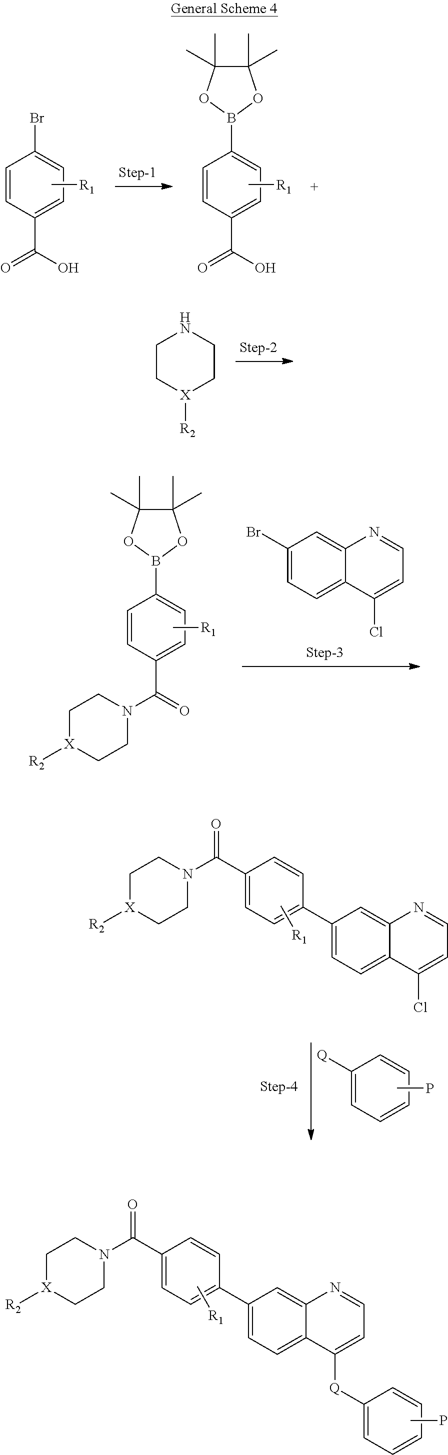

### EXAMPLES

The following synthetic reaction schemes, which are detailed in the Schemes and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including, but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

### Example 1. Synthesis of N-(6-(4-((4-methylpiperazin-1-yl)methyl)phenyl)quinolin-4-yl)benzo[d]thiazol-5-amine (Compound 1)

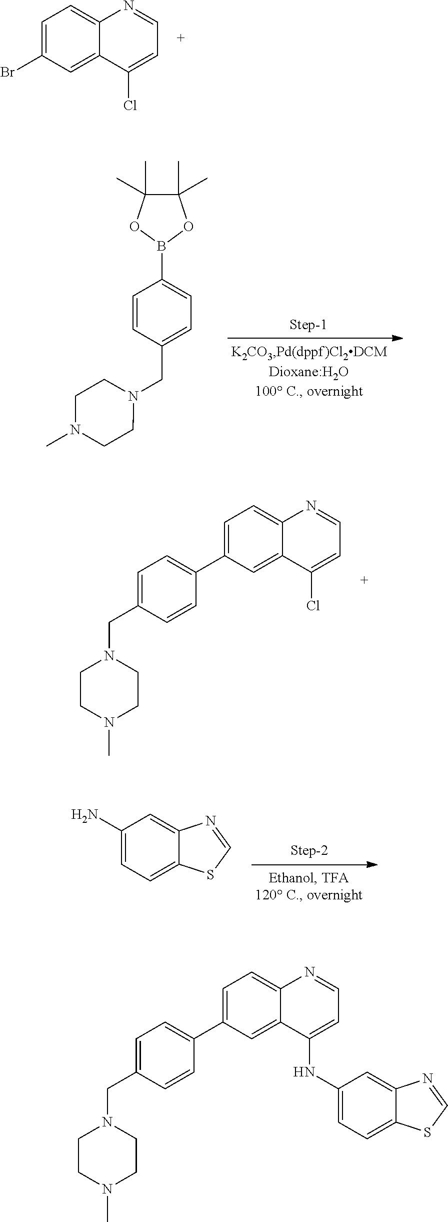

### Step-1: Synthesis of 4-chloro-6-(4-((4-methylpiperazin-1-yl)methyl)phenyl)quinoline

To a stirred solution of 6-bromo-4-chloroquinoline (200 mg, 0.82 mmol, 1.0 eq) in 1,4-dioxane (8.0 mL):water (2.0 ml), was added 1-methyl-4-(4-(4,4,5,5-tetramethyl-1,3,2-dioxaborolan-2-yl)benzyl)piperazine (287 mg, 0.91 mmol, 1.1 eq) followed by addition of K₂CO₃ (170 mg, 1.23 mmol, 1.5 eq) The reaction mixture was purged with nitrogen gas for 10 min, after purging was added PdCl₂dppf·DCM (33 mg, 0.041 mmol, 0.05 eq), and again purged with nitrogen gas for 5 minutes. The reaction mixture was allowed to stir at 100° C. for overnight. The progress of the reaction was monitored by TLC and LCMS. After completion of reaction was added ethyl acetate (3×50 ml) and water (50 ml), The organic layer was concentrated under reduced pressure to afford crude, which was purified by CombiFlash chromatography to obtain 4-chloro-6-(4-(4-methylpiperazin-1-yl)methyl)phenyl)quinoline as a desired product (220 mg, 75.8%), LCMS: 352[M+1].

### Step-2: Synthesis of N-(6-(4-((4-methylpiperazin-1-yl)methyl)phenyl)quinolin-4-yl)benzo[d]thiazol-5-amine

To a stirred solution of 4-chloro-6-(4-((4-methylpiperazin-1-yl)methyl)phenyl)quinoline (120 mg, 0.341 mmol, 1.0 eq) in ethanol (5.0 mL), was added benzo[d]thiazol-5-amine (77 mg, 0.511 mmol, 1.5 eq) and TFA (4 drop). The resultant reaction mixture was allowed to stir at 120° C. for overnight. Progress of the reaction was monitored by TLC and LCMS. After completion, solvent was evaporated, was added Saturated NaHCO3 solution (30 ml), extracted with DCM (3×50 ml). The separated organic layer was concentrated under reduced pressure to afford crude, which was purified by reverse phase chromatography to obtain N-(6-(4-(4-methylpiperazin-1-yl)methyl)phenyl)quinolin-4-yl). benzo[d]thiazol-5-amine as a desired compound (11 mg, 7%). LCMS: 466 [M+1]+, ¹H NMR (400 MHz, DMSO-d₆): δ 9.43 (s, 1H), 8.72 (s, 1H), 8.47 (d, J=5.2 Hz, 1H), 8.21 (d, J=9.1 Hz, 2H), 8.11-8.02 (m, 2H), 7.96 (d, J=8.6 Hz, 1H), 7.86 (d, J=8.1 Hz, 2H), 7.57 (d, J=7.2 Hz, 1H), 7.45 (d, J=8.1 Hz, 2H), 7.03 (d, J=5.2 Hz, 1H), 3.53 (s, 2H) 2.42 (br. s., 8H), 2.21 (s, 3H).

### Example 2. Synthesis of N-(3-fluoro-4-(1-methylpiperidin-4-yl)phenyl)-7-(4-methoxyphenyl)quinolin-4-amine (Compound 2)

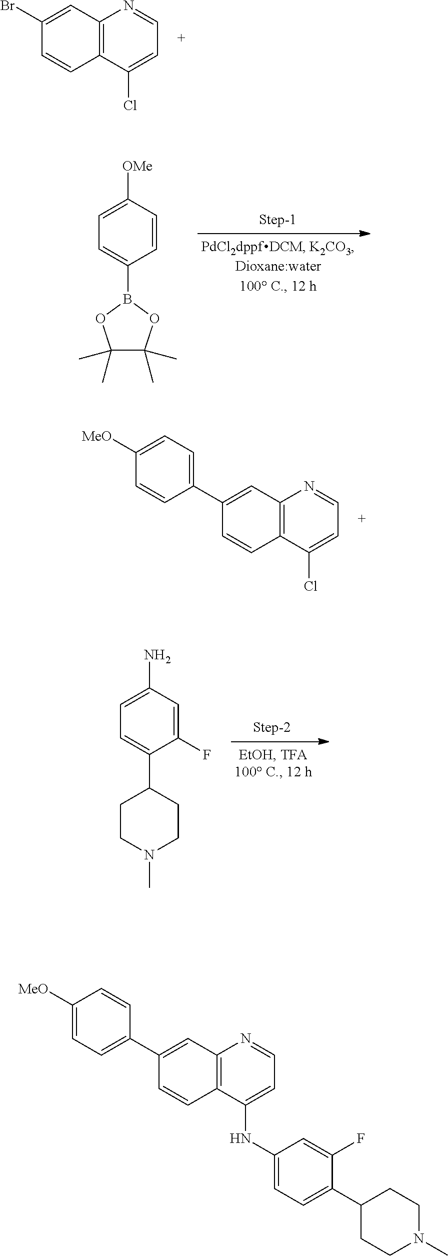

### Step-1: Synthesis of 4-chloro-7-(4-methoxyphenyl)quinoline

To a stirred solution of 7-bromo-4-chloroquinoline (500 mg, 2.06 mmol, 1.0 eq) in 1,4-dioxane (5.0 mL), was added 2-(4-methoxyphenyl)-4,4,5,5-tetramethyl-1,3,2-dioxaborolane (483 mg, 2.06 mmol, 1.0 eq) followed by addition of K₂CO₃ (712 mg, 5.16 mmol, 2.5 eq) and water (1.0 ml). The reaction mixture was purged with nitrogen gas for 10 min, followed by addition of PdCl₂dppf·DCM (84 mg, 0.10 mmol, 0.05 eq), and again purged with nitrogen gas for 5 minutes. The resultant reaction mixture was allowed to stir at 100° C. for 12 h. The progress of the reaction was monitored by TLC, LCMS, and NMR. After completion of the reaction, reaction mixture was filtered through celite bed and washed with ethyl acetate (15 mL). The organic layer was concentrated under reduce pressure to afford crude, which was purified by CombiFlash column purification (Elution: 0-50% EtOAc in Hexane) to get the title compound (420 mg, 75.5%), LCMS: 270.2 [M+1].

### Step-2: Synthesis of N-(3-fluoro-4-(1-methylpiperidin-4-yl)phenyl)-7-(4-methoxyphenyl)quinolin-4-amine

To a stirred solution of 4-chloro-7-(4-methoxyphenyl)quinoline (100 mg, 0.37 mmol, 1.0 eq) in ethanol (2.0 mL), was added 3-fluoro-4-(1-methylpiperidin-4-yl)aniline (92 mg, 0.44 mmol, 1.2 eq) and TFA (1 drop). The resultant reaction mixture was allowed to stir at 100° C. for 12 h. The progress of the reaction was monitored by TLC, LCMS, and NMR. After completion, the reaction mixture was concentrated under reduced pressure to afford crude, which was purified by reverse phase chromatography to get the title compound (76.0 mg, 46.6%), LCMS: 442.4 [M+1]^{+ 1}H NMR (400 MHz, DMSO-d₆) δ ppm 1.66-1.77 (m, 4H) 1.92-2.02 (m, 2H) 2.20 (s, 3H) 2.67 (m, 1H) 2.88 (d, J=10.97 Hz, 2H) 3.83 (s, 3H) 7.02 (d, J=5.25 Hz, 1H) 7.08-7.13 (m, 2H) 7.14-7.20 (m, 2H) 7.34 (t, J=8.58 Hz, 1H) 7.79-7.90 (m, 3H) 8.08 (d, J=1.91 Hz, 1H) 8.39 (d, J=9.06 Hz, 1H) 8.50 (d, J=5.25 Hz, 1H) 9.04 (s, 1H).

### Example 3. Synthesis of 7-(2-fluorophenyl)-N-(5-(1-methylpiperidin-4-yl)pyridin-2-yl)quinolin-4-amine (Compound 3)

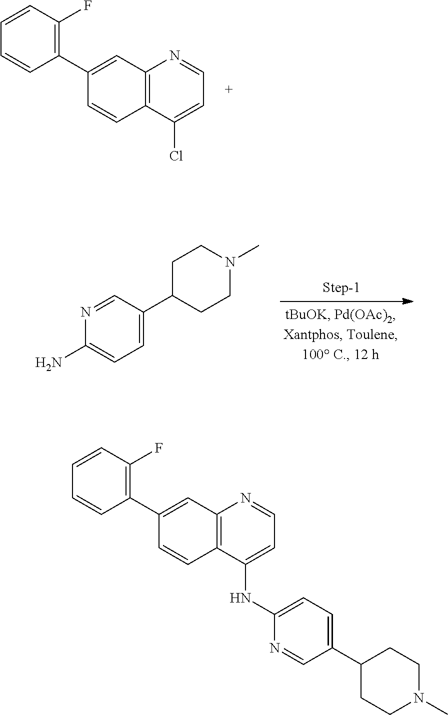 [SMILES: Fc1ccccc1-c1ccc2c(Cl)ccnc2c1 ; CN1CCC(c2ccc(N)nc2)CC1 ; CN1CCC(c2ccc(Nc3ccnc4cc(-c5ccccc5F)ccc34)nc2)CC1]

### Step-1: Synthesis of 7-(2-fluorophenyl)-N-(5-(1-methylpiperidin-4-yl)pyridin-2-yl)quinolin-4-amine

To a stirred solution of 4-chloro-7-(2-fluorophenyl)quinoline (100 mg, 0.388 mmol, 1.0 eq) in Toluene (2.0 mL), was added 5-(1-methylpiperidin-4-yl)pyridin-2-amine (82 mg, 0.42 mmol, 1.1 eq) followed by addition of tBuoK (108 mg, 0.97 mmol, 2.5 eq). The reaction mixture was purged with nitrogen gas for 10 minutes, followed by the addition of Pd(OAc)2 (17 mg, 0.077 mmol, 0.2 eq) and Xantphos (22 mg, 0.033 mmol, 0.1 eq), again purged with nitrogen gas for 5 minutes. The resultant reaction mixture was allowed to stir at 100° C. for 12 h. The progress of the reaction was monitored by TLC, LCMS, and NMR. After completion of the reaction, reaction mixture was filtered through celite bed and washed with ethyl acetate (15 mL). The organic layer was concentrated under reduce pressure to afford crude, which was purified by reverse phase chromatography to get the title compound (41.0 mg, 25.6%). LCMS: 413.4 [M+1]⁺ And ¹H NMR (400 MHz, DMSO-d₆) δ ppm 1.18-1.28 (m, 4H) 1.67-1.84 (m, 2H) 2.31 (s, J=10.97 Hz, 3H) 2.67 (m, 1H) 2.97 (d, 2H) 7.32-7.44 (m, 3H) 7.47-7.53 (m, 1H) 7.63-7.81 (m, 3H) 8.08 (s, 1H) 8.24 (d, J=2.38 Hz, 1H) 8.40 (d, J=5.25 Hz, 1H) 8.59 (d, J=9.06 Hz, 1H) 8.67 (d, J=5.25 Hz, 1H) 9.44 (s, 1H).

### Example 4. Synthesis of N4-(benzo[d]thiazol-5-yl)-N6-(3-fluoro-4-(1-methylpiperidin-4-yl)phenyl)quinoline-4,6-diamine (Compound 4)

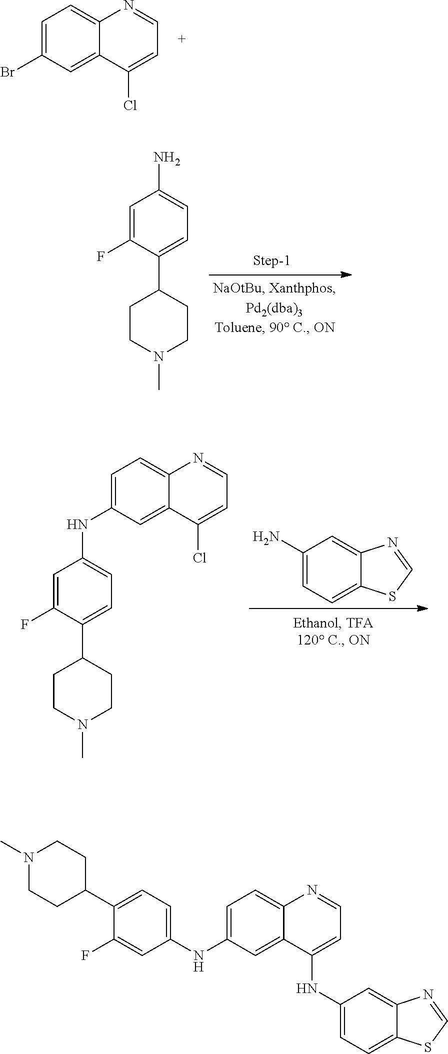 [SMILES: Clc1ccnc2ccc(Br)cc12 ; CN1CCC(c2ccc(N)cc2F)CC1 ; CN1CCC(c2ccc(Nc3ccc4nccc(Cl)c4c3)cc2F)CC1 ; Nc1ccc2scnc2c1 ; CN1CCC(c2ccc(Nc3ccc4nccc(Nc5ccc6scnc6c5)c4c3)cc2F)CC1]

### Step-1: Synthesis of 4-chloro-N-(3-fluoro-4-(1-methylpiperidin-4-yl)phenyl)quinolin-6-amine

To a stirred solution of 6-bromo-4-chloroquinoline (100 mg, 0.416 mmol, 1.0 eq) in Toluene (5.0 mL), was added 3-fluoro-4-(1-methylpiperidin-4-yl)aniline (104 mg, 0.499 mmol, 1.2 eq) followed by addition of tBuONa (100 mg, 1.04 mmol, 2.5 eq). The reaction mixture was purged with nitrogen gas for 10 min, followed by addition of Pd₂(dba)₃ (19 mg, 0.0208 mmol, 0.05 eq), and Xantphos (24 mg, 0.0416 mmol, 0.1 eq), again purged with nitrogen gas for 5 minutes. The resultant reaction mixture was allowed to stir at 90° C. for overnight. Progress of the reaction was monitored by TLC and LCMS. After completion of the reaction, was added water (30 ml) and extracted with DCM (2×50 ml). The separated organic layer was concentrated under reduce pressure to afford crude as a 4-chloro-N-(3-fluoro-4-(1-methylpiperidin-4-yl)phenyl)quinolin-6-amine (145 mg, 95.3%), LCMS: 370[M+1]⁺.

### Step-2: Synthesis of N4-(benzo[d]thiazol-5-yl)-N6-(3-fluoro-4-(1-methylpiperidin-4-yl)phenyl)quinoline-4,6-diamine

To a stirred solution of 4-chloro-N-(3-fluoro-4-(1-methylpiperidin-4-yl)phenyl)quinolin-6-amine (140 mg, 0.378 mmol, 1.0 eq) in ethanol (5.0 mL), was added benzo[d]thiazol-5-amine (68 mg, 0.454 mmol, 1.2 eq) and TFA (0.1 mL). The resultant reaction mixture was allowed to stir at 120° C. for overnight. The progress of the reaction was monitored by TLC and LCMS. After completion, reaction mixture was evaporated to obtain crude which was diluted with aqueous NaHCO₃ solution, was extracted with DCM (2×50 ml). The separated organic layer was concentrated under reduce pressure to afford crude which was purified by reverse phase chromatography to afford N4-(benzo[d]thiazol-5-yl)-N6-(3-fluoro-4-(1-methylpiperidin-4-yl)phenyl)quinoline-4,6-diamine obtain as desired compound (112 mg, 61%). LCMS: 484 [M+1]^{+ 1}H NMR (400 MHz, DMSO-d₆) δ 10.45 (br. s., 1H), 9.50 (s, 1H), 9.06 (s, 1H), 8.41-8.26 (m, 2H), 8.22 (s, 1H), 8.19 (s, 1H), 8.13 (s, 1H), 7.96 (d, J=9.1 Hz, 1H), 7.58 (m., 1H) 7.56 (dd, J=1.9, 8.6 Hz, 1H), 7.15-6.99 (m, 2H), 6.85 (d, J=6.2 Hz, 1H), 3.44 (br. s., 2H), 3.12 (br. s., 2H), 2.74 (s, 3H), 2.67 (m, 1H), 2.03 (br. s., 2H) 1.91 (br. s., 2H).

### Example 5. Synthesis of N-(3-fluoro-4-(1-methylpiperidin-4-yl)phenyl)-7-(2-fluorophenyl)quinolin-4-amine (Compound 5)

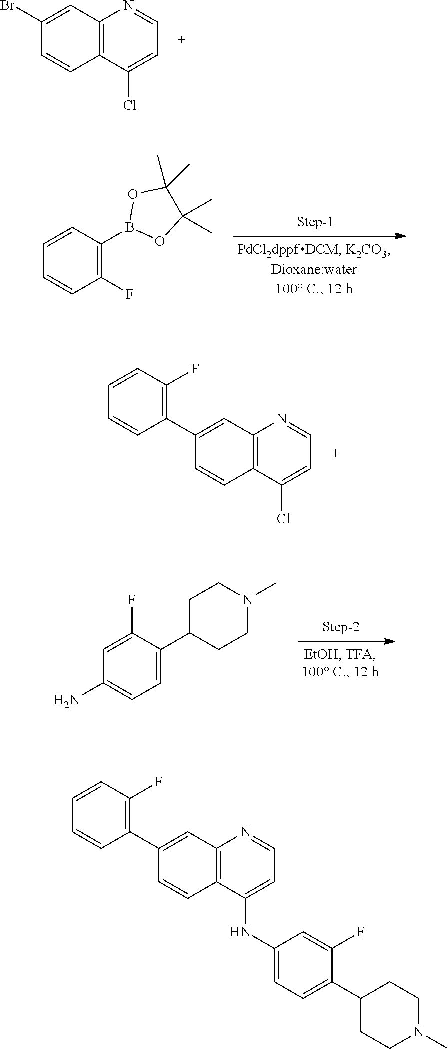 [SMILES: Clc1ccnc2cc(Br)ccc12 ; CC1(C)OB(c2ccccc2F)OC1(C)C ; Fc1ccccc1-c1ccc2c(Cl)ccnc2c1 ; CN1CCC(c2ccc(N)cc2F)CC1 ; CN1CCC(c2ccc(Nc3ccnc4cc(-c5ccccc5F)ccc34)cc2F)CC1]

### Step-1: Synthesis of 4-chloro-7-(2-fluorophenyl)quinoline

To a stirred solution of 7-bromo-4-chloroquinoline (500 mg, 2.06 mmol, 1.0 eq) in 1,4-dioxane (5.0 mL), was added 2-(2-fluorophenyl)-4,4,5,5-tetramethyl-1,3,2-dioxaborolane (460 mg, 2.06 mmol, 1.0 eq) followed by addition of K₂CO₃ (712 mg, 5.16 mmol, 2.5 eq) and water (1.0 ml) The reaction mixture was purged with nitrogen gas for 10 minutes, followed by addition of PdCl₂dppf·DCM (84 mg, 0.10 mmol, 0.05 eq), and again purged with nitrogen gas for 5 minutes. The resultant reaction mixture was allowed to stir at 100° C. for 12 h. The progress of the reaction was monitored by TLC, LCMS, and NMR. After completion of the reaction, reaction mixture was filtered through celite bed and washed with ethyl acetate (15 mL). The organic layer was concentrated under reduce pressure to afford crude, which was purified by CombiFlash column purification (Elution: 0-50% EtOAc in Hexane) to get 4-chloro-7-(2-fluorophenyl)quinoline (400 mg, 75.32%), LCMS: 258.2 [M+1].

### Step-2: Synthesis of N-(3-fluoro-4-(1-methylpiperidin-4-yl)phenyl)-7-(2-fluorophenyl)quinolin-4-amine

To a stirred solution of 4-chloro-7-(2-fluorophenyl)quinoline (100 mg, 0.38 mmol, 1.0 eq) in ethanol (2.0 mL), was added 3-fluoro-4-(1-methylpiperidin-4-yl)aniline (97 mg, 0.46 mmol, 1.2 eq) and TFA (1 drop). The resultant reaction mixture was allowed to stir at 100° C. for 12 h. The progress of the reaction was monitored by TLC, LCMS, and NMR. After completion of reaction, reaction mixture was concentrated under reduce pressure to afford crude, which was purified by reverse phase chromatography to obtain N-(3-fluoro-4-(1-methylpiperidin-4-yl)phenyl)-7-(2-fluorophenyl)quinolin-4-amine as a desired product (45.5 mg, 27.4%), LCMS: 430.4 [M+1]⁺ and ¹H NMR (400 MHz, DMSO-d₆) δ ppm 1.70-1.80 (m, 4H) 2.11-2.22 (m, 2H) 2.30 (s, 3H) 2.72-2.81 (m, 1H) 2.99 (d, J=11.44 Hz, 2H) 7.07 (d, J=5.25 Hz, 1H) 7.08-7.24 (m, 2H) 7.30-7.41 (m, 2H) 7.46-7.56 (m, 1H) 7.67-7.76 (m, 2H) 8.05 (s, 1H) 8.18 (s, 2H) 8.44 (d, J=8.58 Hz, 1H) 8.54 (d, J=5.25 Hz, 1H).

### Example 6. Synthesis of N-(3-fluoro-4-(1-methylpiperidin-4-yl)phenyl)-7-(pyridin-2-yl)quinolin-4-amine (Compound 6)

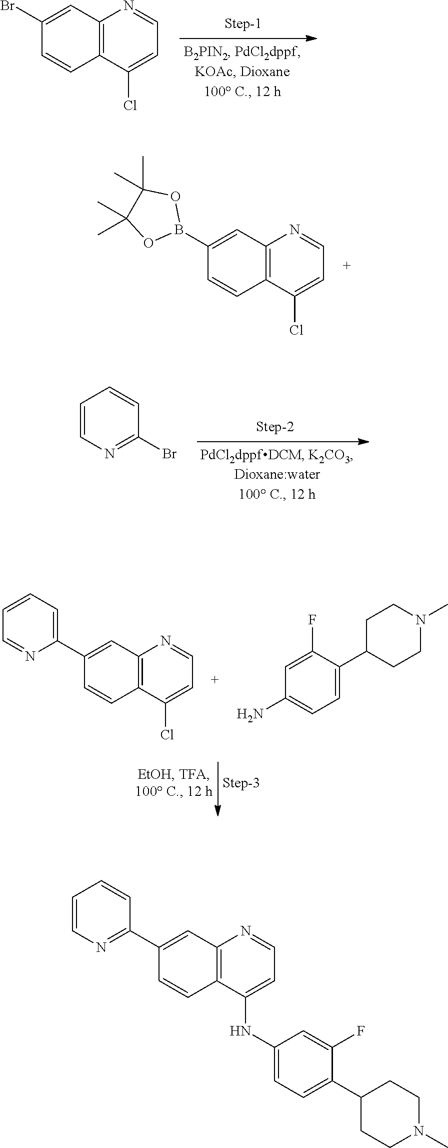 [SMILES: Clc1ccnc2cc(Br)ccc12 ; CC1(C)OB(c2ccc3c(Cl)ccnc3c2)OC1(C)C ; Brc1ccccn1 ; Clc1ccnc2cc(-c3ccccn3)ccc12 ; CN1CCC(c2ccc(N)cc2F)CC1 ; CN1CCC(c2ccc(Nc3ccnc4cc(-c5ccccn5)ccc34)cc2F)CC1]

### Step-1: Synthesis of 4-chloro-7-(4,4,5,5-tetramethyl-1,3,2-dioxaborolan-2-yl)quinoline

To a stirred solution of 7-bromo-4-chloroquinoline (1.0 g, 4.13 mmol, 1.0 eq) in 1,4-dioxane (20 mL), was added bis(pinacolato)diboron (1.57 g, 6.18 mmol, 1.5 eq), followed by the addition of KOAc (607 mg, 6.18 mmol, 1.5 eq). The reaction mixture was purged with nitrogen gas for 10 minutes, followed by the addition of PdCl2dppf (153 mg, 0.20 mmol, 0.05 eq), and again purged with nitrogen gas for 5 minutes. The resultant reaction mixture was allowed to stir at 100° C. for 12 hours. The progress of the reaction was monitored by TLC, LCMS, and NMR. After completion of the reaction, reaction mixture was filtered through celite bed and washed with ethyl acetate (15 mL). The organic layer was concentrated under reduce pressure to afford crude, which was purified by CombiFlash column purification (Elution: 0-50% EtOAc in Hexane) to get the title compound (400 mg, 33.6%). LCMS:290 [M+1].

### Step-2: Synthesis of 4-chloro-7-(pyridin-2-yl)quinoline

To a stirred solution of 2-bromopyridine (500 mg, 3.16 mmol, 1.0 eq) in 1,4-dioxane (5.0 mL), was added 4-chloro-7-(4,4,5,5-tetramethyl-1,3,2-dioxaborolan-2-yl)quinoline (740 mg, 3.16 mmol, 1.0 eq) followed by addition of K₂CO₃ (1.09 g, 7.91 mmol, 2.5 eq) and Water (1.0 ml) The reaction mixture was purged with nitrogen gas for 10 minutes, followed by addition of PdCl₂dppf·DCM (129 mg, 0.15 mmol, 0.05 eq), and again purged with nitrogen gas for 5 minutes. The resultant reaction mixture was allowed to stir at 100° C. for 12 h. The progress of the reaction was monitored by TLC, LCMS, and NMR. After completion of the reaction, reaction mixture was filtered through celite bed and washed with ethyl acetate (15 mL). The organic layer was concentrated under reduce pressure to afford crude, which was purified by CombiFlash column purification (Elution: 0-50% EtOAc in Hexane) to get the title compound (435 mg, 57.1%), LCMS: 241.4 [M+1].

### Step-3: Synthesis of N-(3-fluoro-4-(1-methylpiperidin-4-yl)phenyl)-7-(pyridin-2-yl)quinolin-4-amine

To a stirred solution of 4-chloro-7-(pyridin-2-yl)quinoline (100 mg, 0.41 mmol, 1.0 eq) in ethanol (2.0 mL), was added 3-fluoro-4-(1-methylpiperidin-4-yl)aniline (103 mg, 0.49 mmol, 1.2 eq) and TFA (1 drop). The resultant reaction mixture was allowed to stir at 100° C. for 12 h. The progress of the reaction was monitored by TLC, LCMS, and NMR. After completion, reaction mixture was concentrated under reduce pressure to afford crude, which was purified by reverse phase chromatography to get the title compound (20.2 mg, 11.8%). LCMS: 413.4 [M+1]^{+ 1}H NMR (400 MHz, DMSO-d₆) δ ppm 1.66-1.77 (m, 4H) 1.95-2.05 (m, 2H) 2.22 (s, 3H) 2.67 (m, 1H) 2.89 (d, 2H) 7.06 (d, J=5.25 Hz, 1H) 7.12-7.24 (m, 2H) 7.35 (t, J=8.58 Hz, 1H) 7.43 (dd, J=7.15, 4.77 Hz, 1H) 7.91-7.99 (m, 1H) 8.17-8.23 (m, 1H) 8.30 (d, J=8.58 Hz, 1H) 8.46 (d, J=9.06 Hz, 1H) 8.52-8.61 (m, 2H) 8.76 (d, J=4.77 Hz, 1H) 9.09 (s, 1H).

### Example 7. Synthesis of 7-(4-methoxyphenyl)-N-(5-(1-methylpiperidin-4-yl)pyridin-2-yl)quinolin-4-amine (Compound 7)

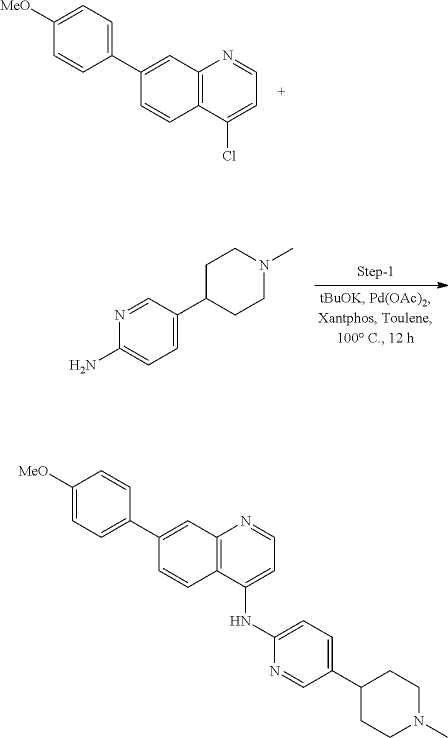 [SMILES: COc1ccc(-c2ccc3c(Cl)ccnc3c2)cc1 ; CN1CCC(c2ccc(N)nc2)CC1 ; COc1ccc(-c2ccc3c(Nc4ccc(C5CCN(C)CC5)cn4)ccnc3c2)cc1]

### Step-1: Synthesis of 7-(4-methoxyphenyl)-N-(5-(1-methylpiperidin-4-yl)pyridin-2-yl)quinolin-4-amine

To a stirred solution of 4-chloro-7-(4-methoxyphenyl)quinoline (100 mg, 0.37 mmol, 1.0 eq) in Toluene (2.0 mL), was added 5-(1-methylpiperidin-4-yl)pyridin-2-amine (78 mg, 0.40 mmol, 1.1 eq) followed by addition of tBuoK (104 mg, 0.92 mmol, 2.5 eq). The reaction mixture was purged with nitrogen gas for 10 min., followed by addition of Pd(OAc)₂ (17 mg, 0.074 mmol, 0.2 eq), and Xantphos (21 mg, 0.037 mmol, 0.1 eq), again purged with nitrogen gas for 5 minutes. The resultant reaction mixture was allowed to stir at 100° C. for 12 h. The progress of the reaction was monitored by TLC, LCMS, and NMR. After completion of the reaction, reaction mixture was filtered through celite bed and washed with ethyl acetate (15 mL). The organic layer was concentrated under reduce pressure to afford crude, which was purified by reverse phase chromatography to obtain 7-(4-methoxyphenyl)-N-(5-(1-methylpiperidin-4-yl)pyridin-2-yl)quinolin-4-amine as a desired compound (20.0 mg, 12.7%). LCMS: 425.4 [M+1]⁺ And ¹H NMR (400 MHz, DMSO-d₆) δ ppm 1.68-1.85 (m, 4H) 2.22 (t, J=11.21 Hz, 2H) 2.34 (s, 3H) 2.67 (m, 1H) 3.02 (d, J=10.49 Hz, 2H) 3.83 (s, 3H) 7.10 (m, J=9.06 Hz, 2H) 7.34 (d, J=8.11 Hz, 1H) 7.67 (dd, J=8.82, 2.15 Hz, 1H) 7.83 (m, J=8.58 Hz, 2H) 7.89 (dd, J=8.58, 1.91 Hz, 1H) 8.09-8.16 (m, 1H) 8.23 (d, J=2.38 Hz, 1H) 8.33 (d, J=5.25 Hz, 1H) 8.54 (d, J=9.06 Hz, 1H) 8.63 (d, J=5.72 Hz, 1H) 9.40 (br. s., 1H).

### Example 8. Synthesis of N-(5-(1-methylpiperidin-4-yl)pyridin-2-yl)-7-(pyridin-2-yl)quinolin-4-amine (Compound 8)

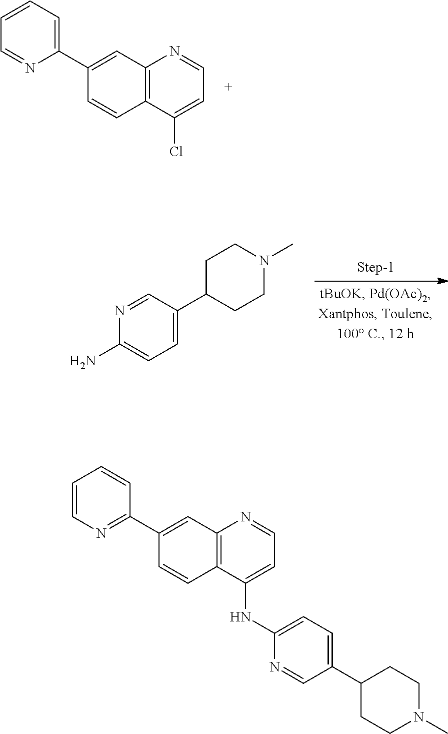 [SMILES: Clc1ccnc2cc(-c3ccccn3)ccc12 ; CN1CCC(c2ccc(N)nc2)CC1 ; CN1CCC(c2ccc(Nc3ccnc4cc(-c5ccccn5)ccc34)nc2)CC1]

### Step-1: Synthesis of N-(5-(1-methylpiperidin-4-yl)pyridin-2-yl)-7-(pyridin-2-yl)quinolin-4-amine

To a stirred solution of 4-chloro-7-(pyridin-2-yl)quinoline (100 mg, 0.41 mmol, 1.0 eq) in Toluene (2.0 mL), was added 5-(1-methylpiperidin-4-yl)pyridin-2-amine (87 mg, 0.45 mmol, 1.1 eq) followed by addition of tBuoK (115 mg, 1.02 mmol, 2.5 eq). The reaction mixture was purged with nitrogen gas for 10 min., followed by addition of Pd(OAc)₂ (18 mg, 0.083 mmol, 0.2 eq), and Xantphos (24 mg, 0.041 mmol, 0.1 eq), again purged with nitrogen gas for 5 minutes. The resultant reaction mixture was allowed to stir at 100° C. for 12 h. The progress of the reaction was monitored by TLC, LCMS, and NMR. After completion of the reaction, reaction mixture was filtered through celite bed and washed with ethyl acetate (15 mL). The organic layer was concentrated under reduce pressure to afford crude, which was purified by reverse phase chromatography to obtain N-(5-(1-methylpiperidin-4-yl)pyridin-2-yl)-7-(pyridin-2-yl)quinolin-4-amine as a desired compound (20.0 mg, 12.8%). LCMS: 396.4 [M+1]^{+ 1}H NMR (400 MHz, DMSO-d₆) δ ppm 1.57-1.80 (m, 4H) 1.88-2.03 (m, 2H) 2.20 (s, 3H) 2.67 (m, 1H) 2.87 (d, J=11.44 Hz, 2H) 7.34 (d, J=8.58 Hz, 1H) 7.44 (d, J=5.25 Hz, 1H) 7.68 (dd, J=8.58, 2.38 Hz, 1H) 7.96 (td, J=7.75, 1.67 Hz, 1H) 8.17-8.25 (m, 2H) 8.32 (dd, J=8.82, 1.67 Hz, 1H) 8.39 (d, J=5.72 Hz, 1H) 8.54-8.62 (m, 2H) 8.67 (d, J=5.25 Hz, 1H) 8.76 (d, J=4.29 Hz, 1H) 9.43 (s, 1H).

### Example 9. Synthesis of N4-(benzo[d]thiazol-5-yl)-N6-(5-(1-methylpiperidin-4-yl)pyridin-2-yl)quinoline-4,6-diamine (Compound 9)

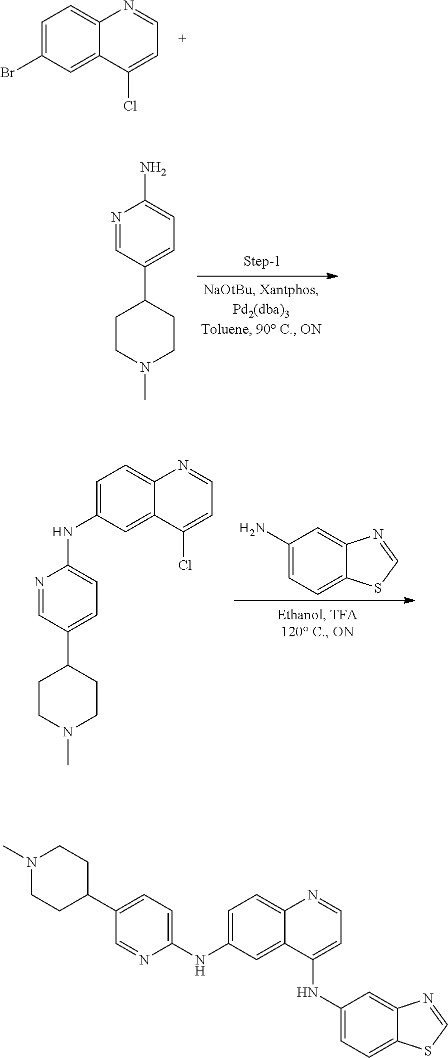 [SMILES: Clc1ccnc2ccc(Br)cc12 ; CN1CCC(c2ccc(N)nc2)CC1 ; CN1CCC(c2ccc(Nc3ccc4nccc(Cl)c4c3)nc2)CC1 ; Nc1ccc2scnc2c1 ; CN1CCC(c2ccc(Nc3ccc4nccc(Nc5ccc6scnc6c5)c4c3)nc2)CC1]

Step-1: Synthesis of 4-chloro-N-(5-(1-methylpiperidin-4-yl)pyridin-2-yl)quinolin-6-amine

To a stirred solution of 6-bromo-4-chloroquinoline (100 mg, 0.416 mmol, 1.0 eq) in Toluene (5.0 mL), was added 5-(1-methylpiperidin-4-yl)pyridin-2-amine (95 mg, 0.499 mmol, 1.2 eq) followed by addition of tBuoNa (100 mg, 1.04 mmol, 2.5 eq). The reaction mixture was purged with nitrogen gas for 10 min, followed by addition of Pd₂(dba)₃ (19 mg, 0.0208 mmol, 0.05 eq), and Xantphos (24 mg, 0.0416 mmol, 0.1 eq), again purged with nitrogen gas for 5 minutes. The resultant reaction mixture was allowed to stir at 90° C. for overnight. The Progress of the reaction was monitored by TLC and LCMS. After completion of the reaction, was added water (30 ml) and extracted with DCM (2×50 ml). The separated organic layer was concentrated under reduce pressure to afford crude as a 4-chloro-N-(5-(1-methylpiperidin-4-yl)pyridin-2-yl)quinolin-6-amine desired product used to next step (140 mg, 96.5%), LCMS: 353 [M+1]⁺.

### Step-2: Synthesis of N4-(benzo[d]thiazol-5-yl)-N6-(5-(1-methylpiperidin-4-yl)pyridin-2-yl)quinoline-4,6-diamine

To a stirred solution of 4-chloro-N-(5-(1-methylpiperidin-4-yl)pyridin-2-yl)quine)in-6-amine (140 mg, 0.397 mmol, 1.0 eq) in ethanol (5.0 mL), was added benzo[d]thiazol-5-amine (72 mg, 0.477 mmol, 1.2 eq) and TFA (0.1 mL). The resultant reaction mixture was allowed to stir at 120° C. for overnight. The progress of the reaction was monitored by TLC and LCMS. After completion, solvent was evaporated under reduce pressure to afford crude which was purified by reverse phase chromatography to obtain N4-(benzo[d]thiazol-5-yl)-N6-(5-(1-methylpiperidin-4-yl)pyridin-2-yl)quinoline-4,6-diamine as a desired compound (123 mg, 53.4%), LCMS: 467 [M+1]⁺, H NMR (400 MHz, DMSO-d₆) δ 9.39 (s, 1H), 9.22 (s, 1H), 8.62-8.54 (m, 1H), 8.35 (d, J 5.2 Hz, 1H), 8.19 (s, 1H), 8.13 (d, J 8.6 Hz, 1 H), 8.10-8.01 (m, 1H), 7.95 (d, J=1.9 Hz, 1H), 7.89-7.74 (m, 2H), 7.59-7.42 (m, 2H), 7.03 (d, J=4.8 Hz, 1H), 6.96-6.84 (m, 1H), 2.96 (s, 2H), 2.67 (m, 1H), 2.35-2.22 (m, 3H), 2.13 (t, J=10.7 Hz, 2H), 1.79-1.64 (m, 4H).

### Example 10. Synthesis of 7-(3-fluoro-4-((4-methylpiperazin-1-yl)methyl)phenyl)-4-(p-tolyloxy)quinoline (Compound 10)

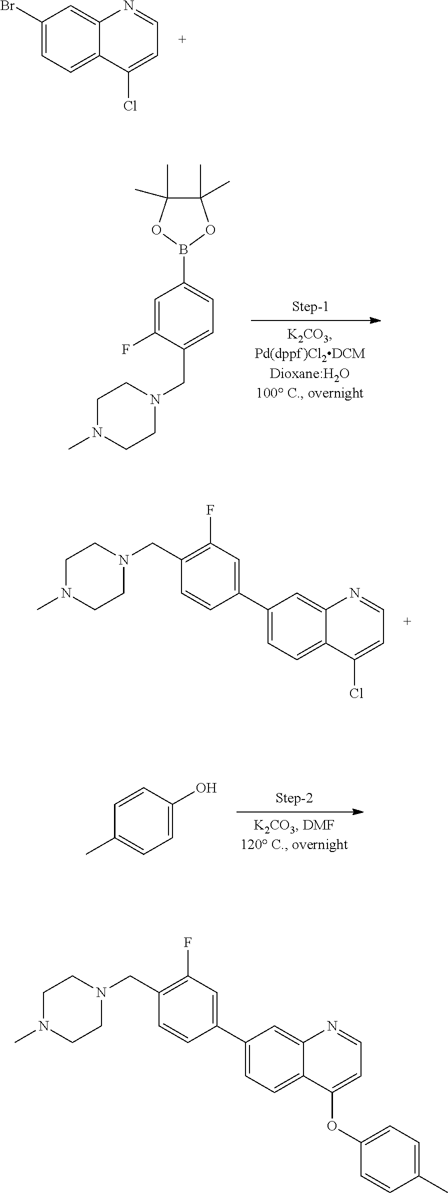

### Step-1: Synthesis of 4-chloro-7-(3-fluoro-4-((4-methylpiperazin-1-yl)methyl)phenyl)quinoline

To a stirred solution of 7-bromo-4-chloroquinoline (300 mg, 1.23 mmol, 1.0 eq) in 1,4-dioxane (4.0 mL):water (1.0 ml), was added 1-(2-fluoro-4-(4,4,5,5-tetramethyl-1,3,2-dioxaborolan-2-yl)benzyl)-4-methylpiperazine (496 mg, 1.48 mmol, 1.2 eq) followed by addition of K₂CO₃ (256 mg, 1.85 mmol, 1.5 eq) The reaction mixture was purged with nitrogen gas for 10 minutes, followed by addition of PdCl₂dppf·DCM (51 mg, 0.628 mmol, 0.5 eq), and again purged with nitrogen gas for 5 minutes. The resultant reaction mixture was allowed to stir at 100° C. for overnight. The progress of the reaction was monitored by TLC and LCMS. After completion of the reaction, was added water (250 ml) and extracted with ethyl acetate (3×150 ml). The separated organic layer was concentrated under reduce pressure to afford crude 4-chloro-7-(3-fluoro-4-((4-methylpiperazin-1-yl)methyl)phenyl)quinoline as a desired product (180 mg, 39.4%), LCMS: 370[M+1].

### Step-2: Synthesis of 7-(3-fluoro-4-((4-methylpiperazin-1-yl)methyl)phenyl)-4-(p-tolyloxy)quinoline

To a stirred solution of 4-chloro-7-(3-fluoro-4-((4-methylpiperazin-1-yl)methyl)phenyl)quinoline (180 mg, 0.487 mmol, 1.0 eq) in DMF (3.0 mL), was added p-cresol (105 mg, 0.975 mmol, 2.0 eq) and K₂CO₃ (201 mg, 1.46 mmol, 3.0 eq) and allowed to stir at 120° C. for overnight. The resultant reaction mixture was allowed to stir at 120° C. for overnight. The progress of the reaction was monitored by TLC and LCMS. After completion, water was added at room temperature and extracted with DCM (3×50 ml). The separated organic layer was concentrated under reduce pressure to afford crude which was purified by CombiFlash chromatography to obtain 7-(3-fluoro-4-((4-methylpiperazin-1-yl)methyl)phenyl)-4-(p-tolyloxy)quinoline as a desired compound (60 mg, 18%), LCMS: 442 [M+1]⁺, ¹H NMR (400 MHz, DMSO-d₆) δ 8.72 (d, J=4.8 Hz, 1H), 8.39 (d, J=8.6 Hz, 1H), 8.17 (s, 1H), 7.84 (d, J=8.6 Hz, 1H), 7.68 (t, J=8.1 Hz, 1H), 7.42-7.25 (m, 4H), 7.21 (d, J=8.1 Hz, 2H), 6.60 (d, J=5.2 Hz, 1H), 3.55 (s, 2H), 2.43 (s, 3H), 2.37 (s, 8H), 2.18 (s, 3H).

### Example 11. Synthesis of N-(6-(2-fluoro-4-((4-methylpiperazin-1-yl)methyl)phenyl)quinolin-4-yl)benzo[d]thiazol-5-amine (Compound 11)

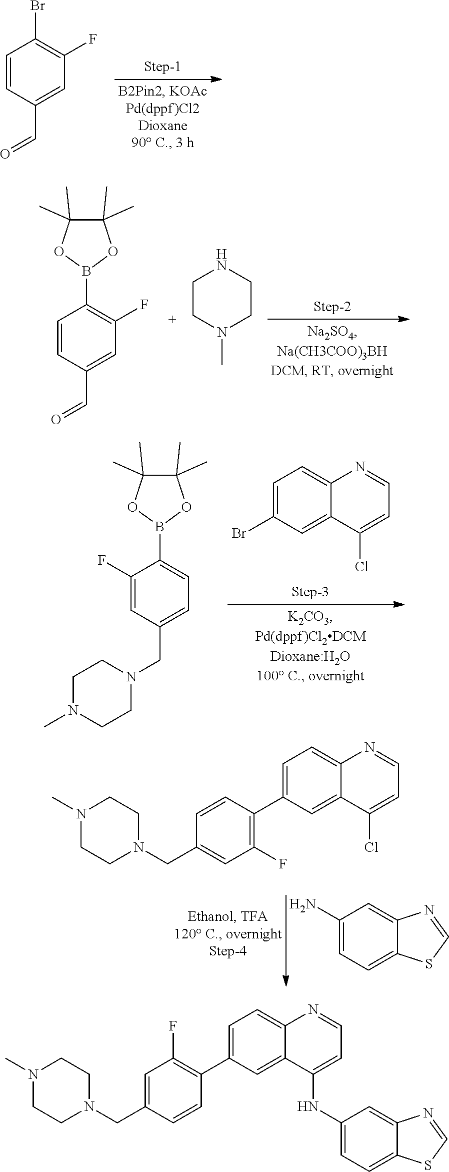

### Step-1: Synthesis of 3-fluoro-4-(4,4,5,5-tetramethyl-1,3,2-dioxaborolan-2-yl)benzaldehyde

To a stirred solution of 4-bromo-3-fluorobenzaldehyde (1.0 g, 4.92 mmol, 1.0 eq) in 1,4-dioxane (10 mL), was added Bis(pinacolato)diboron (1.87 g, 7.38 mmol, 1.5 eq) followed by addition of KOAc (723 mg, 7.38 mmol, 1.5 eq). The reaction mixture was purged with nitrogen gas for 10 min, followed by addition of PdCl₂dppf (180 mg, 0.246 mmol, 0.05 eq), and again purged with nitrogen gas for 5 minutes. The resultant reaction mixture was allowed to stir at 90° C. for 3 h. The progress of the reaction was monitored by TLC. After completion of the reaction, was added water (250 ml) and extracted with ethyl acetate (3×150 ml). The separated organic layer was concentrated under reduce pressure to afford crude which was purified by CombiFlash chromatography to afford 3-fluoro-4-(4,4,5,5-tetramethyl-1,3,2-dioxaborolan-2-yl)benzaldehyde as a title compound (600 mg, 48.7%), LCMS: 251 [M+1].

### Step-2: Synthesis of 1-(3-fluoro-4-(4,4,5,5-tetramethyl-1,3,2-dioxaborolan-2-yl)benzyl)-4-methylpiperazine

To a stirred solution of 3-fluoro-4-(4,4,5,5-tetramethyl-1,3,2-dioxaborolan-2-yl)benzaldehyde (600 mg, 2.4 mmol, 1.0 eq) in DCM (20 ml), was added Na₂SO₄ (682 mg, 4.8 mmol, 2.0 eq), 1-methylpiperazine (360 mg, 3.6 mmol, 1.5 eq) under nitrogen atmosphere and allowed to stir at RT for 1 hr. After 1 h, was added Sodium triacetoxyborohydride (763 mg, 3.6 mmol, 1.5 eq) portion wise to reaction mixture at 0° C. After addition, the reaction mixture was allowed to stir at RT for overnight. The progress of the reaction was monitored by LCMS. After completion of the reaction, saturated NaHCO₃ (50 ml) was added and extracted with DCM (3×150 ml). The separated organic layers were washed with aq. NaHCO₃ (50 ml) and then brine (50 ml), dried over Na2SO4, and concentrated under reduced pressure to afford 1-(3-fluoro-4-(4,4,5,5-tetramethyl-1,3,2-dioxaborolan-2-yl)benzyl)-4-methylpiperazine as the desired product (502 mg, 62.5%), LCMS: 335[M+1].

### Step-3: Synthesis of 4-chloro-6-(2-fluoro-4-((4-methylpiperazin-1-yl)methyl)phenyl)quinoline

To a stirred solution of 6-bromo-4-chloroquinoline (300 mg, 1.23 mmol, 1.0 eq) in 1,4-dioxane (4.0 mL) and water (1.0 ml), was added 1-(3-fluoro-4-(4,4,5,5-tetramethyl-1,3,2-dioxaborolan-2-yl)benzyl)-4-methylpiperazine (496 mg, 1.48 mmol, 1.2 eq), followed by the addition of K2CO3 (256 mg, 1.85 mmol, 1.5 eq) The reaction mixture was purged with nitrogen gas for 10 minutes, followed by the addition of PdCl2dppf·DCM (51 mg, 0.615 mmol, 0.5 eq), and again purged with nitrogen gas for 5 minutes. The resultant reaction mixture was allowed to stir at 100° C. overnight. The progress of the reaction was monitored by TLC and LCMS. After completion of the reaction, the workup of the reaction was carried out using ethyl acetate (3×150 ml) and water (250 ml). The separated organic layer was concentrated under reduced pressure to afford crude 4-chloro-6-(2-fluoro-4-((4-methylpiperazin-1-yl)methyl)phenyl)quinoline as the desired product (170 mg, 37.2%), LCMS: 370 [M+1].

### Step-4: Synthesis of N-(6-(2-fluoro-4-((4-methylpiperazin-1-yl)methyl)phenyl)quinolin-4-yl)benzo[d]thiazol-5-amine

To a stirred solution of 4-chloro-6-(2-fluoro-4-((4-methylpiperazin-1-yl)methyl)phenyl)quinoline (100 mg, 0.271 mmol, 1.0 eq) in ethanol (10.0 mL), was added benzo[d]thiazol-5-amine (49 mg, 0.325 mmol, 1.2 eq) and TFA (0.2 mL). The resultant reaction mixture was allowed to stir at 120° C. for overnight. The progress of the reaction was monitored by TLC and LCMS. After completion, reaction mixture was evaporated, was added saturated NaHCO₃ (30 ml) and extracted with DCM (2×50 ml). The separated organic layer was concentrated under reduce pressure to afford crude which was purified by CombiFlash chromatography to get N-(6-(2-fluoro-4-((4-methylpiperazin-1-yl)methyl)phenyl)quinolin-4-yl)benzo[d]thiazol-5-amine (24 mg, 18%), LCMS: 484 [M+1]⁺, ¹H NMR (400 MHz, DMSO-d₆) δ 9.43 (s, 1H), 9.29 (br. s., 1H), 8.61 (s, 1H), 8.51 (d, J=5.2 Hz, 1H), 8.20 (d, J=8.6 Hz, 1H), 8.03 (d, J=1.9 Hz, 1H), 7.97 (d, J=8.6 Hz, 1H), 7.89 (d, J=9.1 Hz, 1H), 7.75-7.67 (m, 1H), 7.55 (dd, J=1.9, 8.6 Hz, 1H), 7.38-7.25 (m, 2H), 7.05 (d, J=5.2 Hz, 1H), 3.56 (s, 2H), 2.44-2.33 (br. s., 8H), 2.23 (br. s., 3H).

### Example 12. Synthesis of 4-(4-(4-(benzo[d]thiazol-5-ylamino)quinolin-6-yl)-3-fluorobenzyl)piperazin-2-one (Compound 12)

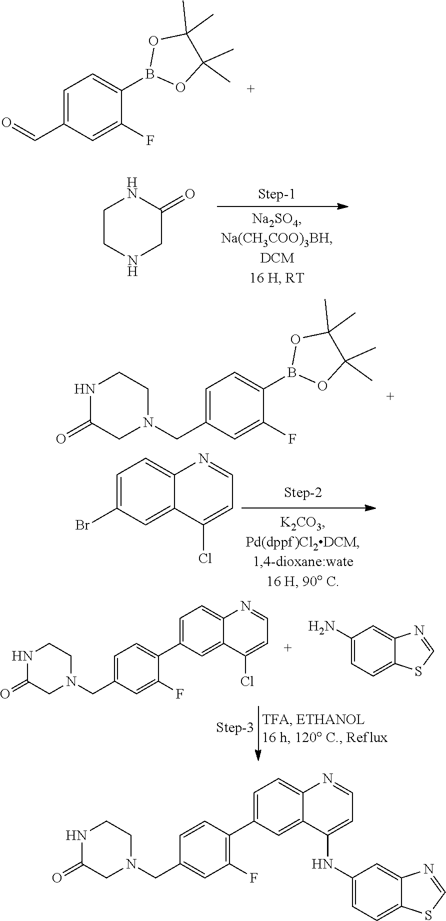

### Step-1: Synthesis of 4-(3-fluoro-4-(4,4,5,5-tetramethyl-1,3,2-dioxaborolan-2-yl)benzyl)piperazin-2-one

To a stirred solution of 3-fluoro-4-(4,4,5,5-tetramethyl-1,3,2-dioxaborolan-2-yl)benzaldehyde (400 mg, 1.60 mmol, 1 eq) and piperazin-2-one (240.22 mg, 2.39 mmol, 1.5 eq) in DCM (15 mL), we added Na₂SO₄ (455 mg, 3.20 mmol, 2.0 eq). Reaction was stirred at RT for 1 h. Further, was added Sodium triacetoxyborohydride (509 mg, 2.4 mmol, 1.5 eq.) portion wise at 0° C. Resulting reaction mass was stirred at RT for 16 h. The Progress of the reaction was monitored by LCMS. After completion of reaction, reaction mixture was diluted with water (50 mL) and extracted with DCM (2×75 mL). Combined organic layers were washed with saturated NaHCO₃ (50 mL) solution. Then, organic layer was dried by Na₂SO₄, filtered and concentrated under reduced pressure to afford 4-(3-fluoro-4-(4,4,5,5-tetramethyl-1,3,2-dioxaborolan-2-yl)benzyl)piperazin-2-one (250 mg, 46.8%). LCMS: 253.3 [Boronic-acid Fragment].

### Step-2: Synthesis of 4-(4-(4-chloroquinolin-6-yl)-3-fluorobenzyl)piperazin-2-one

To a stirred solution of 6-bromo-4-chloroquinoline (150 mg, 0.62 mmol, 1.0 eq) and 4-(3-fluoro-4-(4,4,5,5-tetramethyl-1,3,2-dioxaborolan-2-yl)benzyl)piperazin-2-one (248.6 mg, 0.75 mmol, 1.2 eq) in 1,4-dioxane:water (4 mL:1 mL), was added K₂CO₃ (128.34 mg, 0.93 mmol, 1.5 eq). Reaction was purged with N2 gas for 10 minutes and then was added Pd(dppf)Cl₂·DCM (25.3 mg, 0.031 mmol, 0.05 eq). Resulting reaction mass was stirred at 90° C. for 16 h. The Progress of the reaction was monitored by LCMS and TLC. After completion reaction, reaction mixture was diluted with water (40 mL) and extracted with EtOAc (2×50 mL). Combined organic layers were dried by Na₂SO₄, filtered and concentrated under reduced pressure to afford crude product which was purified by flash chromatography (eluted in 2% MeOH:DCM) to afford 4-(4-(4-chloroquinolin-6-yl)-3-fluorobenzyl)piperazin-2-one (110 mg, 48%). LCMS: 370.3 [M+1]⁺.

### Step-3: Synthesis of 4-(4-(4-(benzo[d]thiazol-5-ylamino)quinolin-6-yl)-3-fluorobenzyl)piperazin-2-one

To a solution of afford 4-(4-(4-chloroquinolin-6-yl)-3-fluorobenzyl)piperazin-2-one (100 mg, 0.271 mmol, 1.0 eq) and benzo[d]thiazol-5-amine (60.9 mg, 0.410 mmol, 1.5 eq) in EtOH (4 mL) was added TFA (0.1 mL) and the reaction mixture was allowed to reflux at 120° C. for 16 h. The progress of the reaction was monitored by TLC and LCMS. After completion of reaction, reaction mixture was concentrated under reduced pressure to obtain crude which was basified with saturated solution of NaHCO₃ (20 mL) and was extracted with DCM (2×35 mL). Combined organic layers were dried by Na2SO4, filtered and concentrated under reduced pressure to afford crude product. Crude product was purified by flash chromatography, eluted in 6% MeOH:DCM to afford 4-(4-(4-(benzo[d]thiazol-5-ylamino)quinolin-6-yl)-3-fluorobenzyl)piperazin-2-one (20 mg, 15.3%). Analytical Data: LCMS: 484.3 [M+1]⁺, ¹H NMR (400 MHz, DMSO-d₆) δ ppm 9.43 (s, 1H) 9.38 (br. s., 1H) 8.63 (s, 1H) 8.51 (d, J=5.25 Hz, 1H) 8.21 (d, J=8.58 Hz, 1H) 8.05 (d, J=1.91 Hz, 1H) 7.97 (s, 1H) 7.88-7.96 (m, 1H) 7.78 (s, 1H) 7.67-7.72 (m, 1H) 7.56 (dd, J=8.82, 1.67 Hz, 1H) 7.35 (s, 1H) 7.33 (s, 1H) 7.05 (d, J=5.72 Hz, 1H) 3.64 (s, 2H) 3.18 (br. s., 2H) 2.96 (s, 2H) 2.61 (d, J=5.25 Hz, 2H).

### Example 13. Synthesis of 4-(4-(4-(benzo[d]thiazol-5-ylamino)quinolin-6-yl)-3-fluorobenzyl)-1-methylpiperazin-2-one (Compound 13)

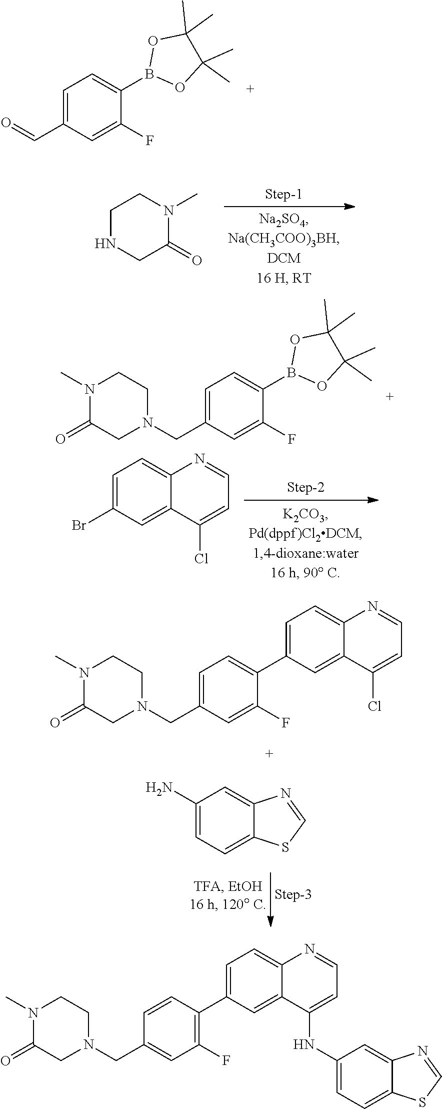

### Step-1: Synthesis of 4-(3-fluoro-4-(4,4,5,5-tetramethyl-1,3,2-dioxaborolan-2-yl)benzyl)-1-methylpiperazin-2-one

To a stirred solution of 3-fluoro-4-(4,4,5,5-tetramethyl-1,3,2-dioxaborolan-2-yl)benzaldehyde (400 mg, 1.60 mmol, 1 eq) and 1-methylpiperazin-2-one (273.96 mg, 2.4 mmol, 1.5 eq) in DCM (15 mL), we added Na₂SO₄ (455 mg, 3.20 mmol, 2.0 eq). Reaction was stirred at RT for 1 h, was added Sodium triacetoxyborohydride (509 mg, 2.4 mmol, 1.5 eq.) portion wise at 0° C. after addition, reaction mixture was stirred at RT for 16 h. The progress of the reaction was monitored by LCMS. After completion of reaction, reaction mixture was diluted with water (75 mL) and was extracted with DCM (2×100 mL). Combined organic layers were washed with saturated NaHCO₃ solution (50 mL). Then, organic layer was dried over anhydrous Na₂SO₄, filtered and concentrated under reduced pressure to afford 4-(3-fluoro-4-(4,4,5,5-tetramethyl-1,3,2-dioxaborolan-2-yl)benzyl)-1-methylpiperazin-2-one (500 mg, 89.9%), LCMS: 267.3 [Boronic-acid Fragment].

### Step-2: Synthesis of 4-(4-(4-chloroquinolin-6-yl)-3-fluorobenzyl)-1-methylpiperazin-2-one

To a stirred solution of 6-bromo-4-chloroquinoline (300 mg, 1.24 mmol, 1.0 eq) and 4-(3-fluoro-4-(4,4,5,5-tetramethyl-1,3,2-dioxaborolan-2-yl)benzyl)-1-methylpiperazin-2-one (516.9 mg, 1.48 mmol, 1.5 eq) in 1,4-dioxane:water (8 mL:2 mL), was added K₂CO₃ (256.7 mg, 1.86 mmol, 1.5 eq). Reaction mixture was purged with N2 gas for 10 minutes and then was added Pd(dppf)Cl2·DCM (50.59 mg, 0.062 mmol, 0.05 eq). After addition, reaction mixture was stirred at 90° C. for 16 h. The progress of the reaction was monitored by LCMS and TLC. After completion of reaction, reaction mixture was diluted with water (75 mL) and was extracted with EtOAc (2×100 mL). Combined organic layers were dried by Na₂SO₄, filtered and concentrated under reduced pressure to afford crude which was purified by flash chromatography (eluted in 2% MeOH:DCM) to afford 4-(4-(4-chloroquinolin-6-yl)-3-fluorobenzyl)-1-methylpiperazin-2-one (200 mg, 42%), LCMS: 384.3 [M+1]⁺.

### Step-3: Synthesis of 4-(4-(4-(benzo[d]thiazol-5-ylamino)quinolin-6-yl)-3-fluorobenzyl)-1-methylpiperazin-2-one

To a solution of 4-(4-(4-chloroquinolin-6-yl)-3-fluorobenzyl)-1-methylpiperazin-2-one (200 mg, 0.523 mmol, 1.0 eq) and benzo[d]thiazol-5-amine (117.5 mg, 0.784 mmol, 1.5 eq) in EtOH (5 mL) was added TFA (0.2 mL) and the reaction mixture was allowed to reflux at 120° C. for 16 h. The progress of the reaction was monitored by TLC and LCMS. After completion, reaction mixture was concentrated under reduced pressure to obtain crude was basified with saturated NaHCO₃ solution (40 mL) and was extracted with DCM (2×50 mL). Combined organic layers were dried over Na₂SO₄, filtered and concentrated under reduced pressure to afford crude which was purified by flash chromatography (eluted in 6% MeOH:DCM) to afford 4-(4-(4-(benzo[d]thiazol-5-ylamino)quinolin-6-yl)-3-fluorobenzyl)-1-methylpiperazin-2-one (85.75 mg, 32.8%), ANALYTICAL DATA: LCMS: 498.3 [M+1]⁺, ¹H NMR (400 MHz, DMSO-d₆) δ ppm 9.43 (s, 1H) 9.30 (br. s., 1H) 8.61 (s, 1H) 8.51 (d, J=4.77 Hz, 1H) 8.20 (d, J=8.58 Hz, 1H) 8.04 (s, 1H) 7.94-8.00 (m, 1H) 7.91 (s, 1H) 7.69-7.74 (m, 1H) 7.55 (d, J=8.58 Hz, 1H) 7.34 (d, J=9.54 Hz, 2H) 7.05 (d, J=5.25 Hz, 1H) 3.63 (s, 2H) 3.20-3.30 (m, 2H) 3.01 (s, 2H) 2.83 (s, 3H) 2.68 (d, J=5.72 Hz, 2H).

### Example 14. Synthesis of N-(6-(2-fluoro-4-(piperidin-1-ylmethyl)phenyl)quinolin-4-yl)benzo[d]thiazol-5-amine (Compound 14)

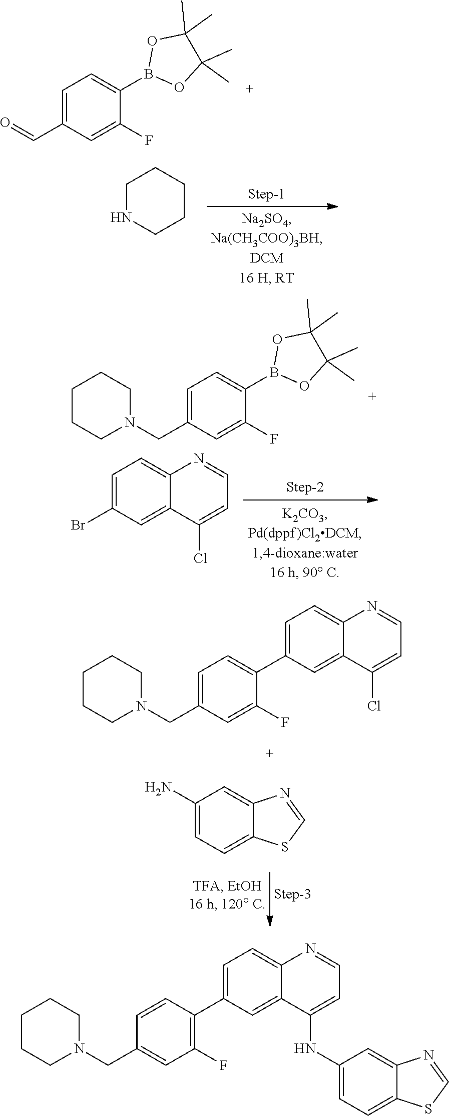

### Step-1: Synthesis of 1-(3-fluoro-4-(4,4,5,5-tetramethyl-1,3,2-dioxaborolan-2-yl)benzyl)piperidine

To a stirred solution of 3-fluoro-4-(4,4,5,5-tetramethyl-1,3,2-dioxaborolan-2-yl)benzaldehyde (400 mg, 1.60 mmol, 1 eq) and piperidine (163.4 mg, 1.92 mmol, 1.2 eq) in DCM (15 mL), was added Na₂SO₄ (455 mg, 3.20 mmol, 2.0 eq). Reaction mixture was stirred at RT for 1 h. Further, was added sodium triacetoxyborohydride (509 mg, 2.4 mmol, 1.5 eq) portion wise at 0° C. After addition, reaction mixture was stirred at RT for 16 h. The progress of the reaction was monitored by LCMS. After completion of reaction, reaction mixture was diluted with water (75 mL) and was extracted with DCM (2×100 mL). Combined organic layers were washed with saturated NaHCO₃ solution (50 mL), Organic layer was dried over anhydrous Na₂SO₄, filtered and concentrated under reduced pressure to afford 1-(3-fluoro-4-(4,4,5,5-tetramethyl-1,3,2-dioxaborolan-2-yl)benzyl)piperidine (400 mg, 78.43%), LCMS: 238.3 [Boronic-acid Fragment].

### Step-2: Synthesis of 4-chloro-6-(2-fluoro-4-(piperidin-1-ylmethyl)phenyl)quinoline

To a stirred solution of 6-bromo-4-chloroquinoline (250 mg, 1.04 mmol, 1.0 eq) and 1-(3-fluoro-4-(4,4,5,5-tetramethyl-1,3,2-dioxaborolan-2-yl)benzyl)piperidine (398.37 mg, 1.23 mmol, 1.2 eq) in 1,4-dioxane:water (6 mL:1.5 mL), was added K₂CO₃ (215.3 mg, 1.56 mmol, 1.5 eq). Reaction mixture was purged with N2 gas for 10 minutes and was added Pd(dppf)Cl2·DCM (42.4 mg, 0.052 mmol, 0.05 eq). Reaction mixture was stirred at 90° C. for 16 h. The progress of the reaction was monitored by LCMS and TLC. After completion of reaction, reaction mixture was diluted with water (50 mL) and was extracted with EtOAc (2×75 mL), combined organic layers were dried over Na2SO4, filtered and concentrated under reduced pressure to afford crude which was purified by flash chromatography (eluted in 2% MeOH:DCM) to afford 4-chloro-6-(2-fluoro-4-(piperidin-1-ylmethyl)phenyl)quinoline (130 mg, 35.3%). LCMS: 355.3 [M+1]⁺.

### Step-3: Synthesis of N-(6-(2-fluoro-4-(piperidin-1-ylmethyl)phenyl)quinolin-4-yl)benzo[d]thiazol-5-amine

To a solution of 4-chloro-6-(2-fluoro-4-(piperidin-1-ylmethyl)phenyl)quinoline (130 mg, 0.37 mmol, 1.0 eq) and benzo[d]thiazol-5-amine (82.6 mg, 0.55 mmol, 1.5 eq) in EtOH (6 mL) was added TFA (0.1 mL) and the reaction mixture was stirred at 120° C. for 16 h. the progress of the reaction was monitored by TLC and LCMS. After completion of reaction, reaction mixture was concentrated under reduced pressure to obtain crude which was basified with saturated NaHCO₃ solution (30 mL) and was extracted with DCM (2×50 mL). Combined organic layers were dried over Na2SO4, filtered and concentrated under reduced pressure to afford which was purified by flash chromatography (eluted in 6% MeOH:DCM) to afford N-(6-(2-fluoro-4-(piperidin-1-ylmethyl)phenyl)quinolin-4-yl)benzo[d]thiazol-5-amine (53.67 mg, 31.3%). ANALYTICAL DATA: LCMS: 469.3 [M+1]⁺, ¹H NMR (400 MHz, DMSO-d₆) δ ppm 9.43 (s, 1H) 9.29 (br. s., 1H) 8.61 (s, 1H) 8.51 (d, J=5.25 Hz, 1H) 8.20 (d, J=8.58 Hz, 1H) 8.03 (d, J=1.91 Hz, 1H) 7.97 (d, J=8.58 Hz, 1H) 7.89 (d, J=9.06 Hz, 1H) 7.68 (t, J=8.11 Hz, 1H) 7.55 (dd, J=8.58, 1.91 Hz, 1H) 7.25-7.33 (m, 2H) 7.05 (d, J=5.25 Hz, 1H) 3.61 (s, 2H) 3.54 (br. s., 4H) 1.50-1.57 (m, 4H) 1.41 (br. s., 2H).

### Example 15. Synthesis of N-(6-(4-(4-methylpiperazin-1-yl)phenyl)quinolin-4-yl)benzo[d]thiazol-5-amine (Compound 15)

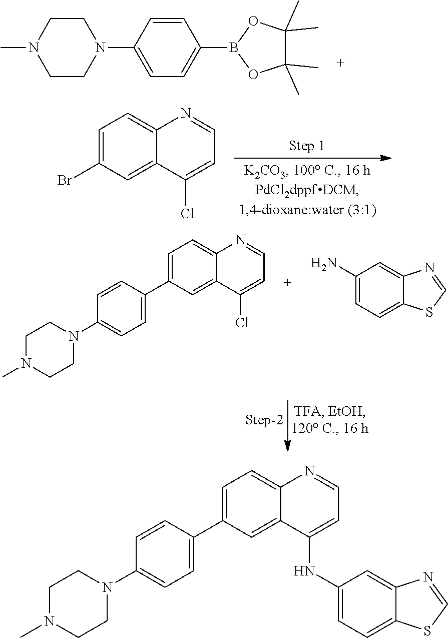

### Step-1: Synthesis of 4-chloro-6-(4-(4-methylpiperazin-1-yl)phenyl)quinoline

To a solution of 1-methyl-4-(4-(4,4,5,5-tetramethyl-1,3,2-dioxaborolan-2-yl)phenyl)piperazine (300 mg, 0.99 mmol, 1 eq) in 1,4-dioxane:water (3.5 mL:1.5 mL) was added K₂CO₃ (274 mg, 1.98 mmol, 2 eq) and 6-bromo-4-chloroquinoline (240 mg, 0.99 mmol, 1 eq). Reaction mixture was purged with nitrogen for 10 min, was added PdCl₂dppf·DCM (40 mg, 0.05 mmol, 0.05 eq). The reaction was heated at 100° C. for 16 h. The progress of reaction was monitored by TLC. After completion of reaction, reaction mixture was diluted with water (30 mL) and extracted with ethyl acetate (2×75 mL), dried over anhydrous sodium sulphate, filtered and concentrated under vacuum to afford crude which was purified by flash chromatography to afford 4-chloro-6-(4-(4-methylpiperazin-1-yl)phenyl)quinoline (280 mg, 83.8%), LCMS: 338[M+1]⁺.

### Step-2: Synthesis of N-(6-(4-(4-methylpiperazin-1-yl)phenyl)quinolin-4-yl)benzo[d]thiazol-5-amine

To a solution of 4-chloro-6-(4-(4-methylpiperazin-1-yl)phenyl)quinoline (100 mg, 0.295 mmol, 1.0 eq) in ethanol (5 mL) was added benzo[d]thiazol-5-amine (66 mg, 0.443 mmol, 1.5 eq) and TFA (0.1 mL). The reaction mixture was heated at 120° C. for 16 h. After completion of reaction, reaction mixture was concentrated under vacuum to obtain crude which was neutralized by saturated sodium bicarbonate solution (20 mL), was extracted with dichloromethane (2×40 mL), dried over anhydrous sodium sulphate, filtered and concentrated under vacuum to afford crude which was purified by flash chromatography to afford N-(6-(4-(4-methylpiperazin-1-yl)phenyl)quinolin-4-yl)benzo[d]thiazol-5-amine (46 mg, 34.5%), LCMS: 452[M+1]⁺, ¹H NMR (400 MHz, DMSO-d₆) D ppm 2.32 (br.s., 4H) 2.24 (s, 3H) 3.23 (br. s., 4H) 7.00 (d, J=4.77 Hz, 1H) 7.09 (d, J=8.58 Hz, 2H) 7.57 (d, J=8.11 Hz, 1H) 7.80 (d, J=9.06 Hz, 2H) 7.91 (d, J=8.58 Hz, 1H) 8.02 (d, J=9.06 Hz, 1H) 8.05 (s, 1H) 8.21 (d, J=8.58 Hz, 1H) 8.43 (d, J=5.72 Hz, 1H) 8.63 (s, 1H) 9.27 (s, 1H) 9.43 (s, 1H).

### Example 16. Synthesis of N-(7-(4-((4-methylpiperazin-1-yl)methyl)phenyl)quinolin-4-yl)benzo[d]thiazol-5-amine (Compound 16)

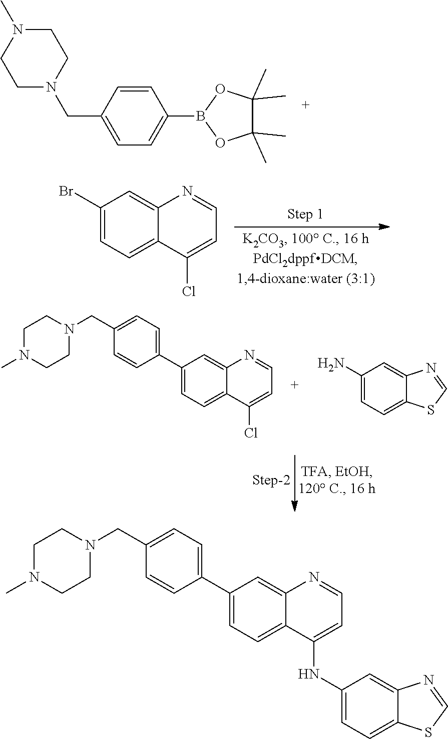

### Step-1: Synthesis of 4-chloro-7-(4-((4-methylpiperazin-1-yl)methyl)phenyl)quinoline

To a solution of 1-methyl-4-(4-(4,4,5,5-tetramethyl-1,3,2-dioxaborolan-2-yl)benzyl)piperazine (500 mg, 1.58 mmol, 1 eq) in 1,4-dioxane:water (3.5 mL:1.5 mL) was added K₂CO₃ (436 mg, 3.16 mmol, 2 eq) and 7-bromo-4-chloroquinoline (383 mg, 1.58 mmol, 1 eq). Reaction mixture was purged with nitrogen for 10 min, was added PdCl2dppf·DCM (64 mg, 0.07 mmol, 0.05 eq). The reaction was heated at 100° C. for 16 h. The progress of reaction was monitored by TLC. After completion of reaction, reaction mixture was diluted with water (50 mL) and extracted with ethyl acetate (2×100 mL), dried over anhydrous sodium sulphate, filtered and concentrated under vacuum to afford crude which was purified by flash chromatography to afford 4-chloro-7-(4-((4-methylpiperazin-1-yl)methyl)phenyl)quinoline (380 mg, 68.4%), LCMS: 352[M+1]⁺.

### Step-2: Synthesis of N-(7-(4-((4-methylpiperazin-1-yl)methyl)phenyl)quinolin-4-yl)benzo[d]thiazol-5-amine

To a solution of 4-chloro-7-(4-((4-methylpiperazin-1-yl)methyl)phenyl)quinoline (100 mg, 0.284 mmol, 1.0 eq) in ethanol (5 mL) was added benzo[d]thiazol-5-amine (64 mg, 0.426 mmol, 1.5 eq) and TFA (0.1 mL). The reaction mixture was heated at 120° C. for 16 h. The progress of reaction was monitored by TLC. After completion of reaction, reaction mixture was concentrated under vacuum to obtain crude which was neutralized by saturated sodium bicarbonate solution (20 mL), was extracted with dichloromethane (2×40 mL), dried over anhydrous sodium sulphate, filtered and concentrated under vacuum to afford crude compound was purified by flash chromatography to afford N-(7-(4-((4-methylpiperazin-1-yl)methyl)phenyl)quinolin-4-yl)benzo[d]thiazol-5-amine (67 mg, 50.7%), LCMS: 466 [M+1]⁺, ¹H NMR (400 MHz, DMSO-d₆) δ ppm 2.14 (s, 3H) 2.48 (s, 4H), 3.15 (s, 4H), 3.55 (s, 2H) 6.93-7.07 (m, 2H) 7.13 (d, J=2.38 Hz, 1H) 7.46 (m, J=7.63 Hz, 1H) 7.56 (d, J=8.58 Hz, 1H) 7.84 (m, J=8.11 Hz, 2H) 7.92 (d, J=8.11 Hz, 1H) 8.04 (s, 1H) 8.15 (s, 1H) 8.20 (d, J=8.58 Hz, 1H) 8.49-8.54 (m, 1H) 9.25 (br. s., 1H) 9.43 (s, 1H).

### Example 17. Synthesis of 2-(4-(4-(4-(benzo[d]thiazol-5-ylamino)quinolin-6-yl)-3-fluorobenzyl)piperazin-1-yl)ethanol (Compound 17)

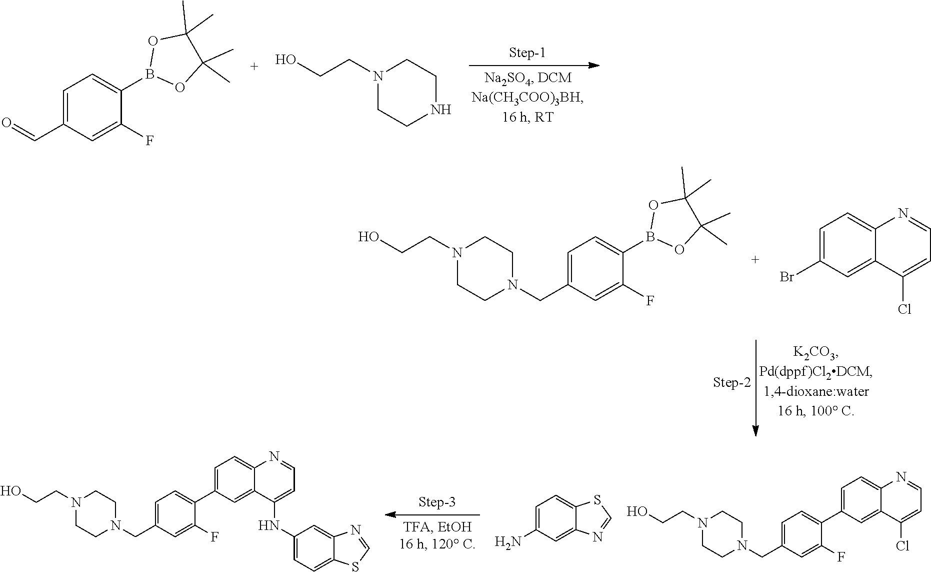

### Step-1: Synthesis of 2-(4-(3-fluoro-4-(4,4,5,5-tetramethyl-1,3,2-dioxaborolan-2-yl)benzyl)piperazin-1-yl)ethanol

To a stirred solution of 3-fluoro-4-(4,4,5,5-tetramethyl-1,3,2-dioxaborolan-2-yl)benzaldehyde (400 mg, 1.60 mmol, 1 eq) and 2-(piperazin-1-yl)ethan-1-ol (312 mg, 2.40 mmol, 1.5 eq) in DCM (15 mL) was added Na₂SO₄ (454 mg, 3.20 mmol, 2.0 eq). Reaction mixture was stirred at RT for 1 h and was added Sodium triacetoxyborohydride (506 mg, 2.4 mmol, 1.5 eq) portion wise at 0° C. After addition, reaction mixture was stirred RT for 16 h. The progress of reaction was monitored by LCMS. After completion of reaction, reaction mixture was diluted with water (50 mL) and was extracted with DCM (2×80 mL). The combined organic layers were washed with saturated NaHCO₃ solution (50 mL). The organic layer was dried over anhydrous sodium sulphate, filtered and concentrated under reduced pressure to afford 2-(4-(3-fluoro-4-(4,4,5,5-tetramethyl-1,3,2-dioxaborolan-2-yl)benzyl)piperazin-1-yl)ethanol (350 mg, 60.13%), LCMS: 283.3 [Boronic-acid Fragment].

### Step-2: Synthesis of 2-(4-(4-(4-chloroquinolin-6-yl)-3-fluorobenzyl)piperazin-1-yl)ethanol

To a stirred solution of 6-bromo-4-chloroquinoline (233 mg, 0.96 mmol, 1.0 eq) and 2-(4-(3-fluoro-4-(4,4,5,5-tetramethyl-1,3,2-dioxaborolan-2-yl)benzyl)piperazin-1-yl)ethan-1-ol (350 mg, 0.96 mmol, 1.0 eq) in 1,4-dioxane:water (3.5 ml:1.5 ml) was added K₂CO₃ (265 mg, 1.92 mmol, 1.5 eq). Reaction mixture was purged with N2 gas for 10 minutes and was added Pd(dppf)Cl₂·DCM (39 mg, 0.048 mmol, 0.05 eq) and the reaction was heated at 100° C. for 16 h. The progress of reaction was monitored by LCMS and TLC. After completion of reaction, reaction mixture was diluted with water (50 mL) and extracted with EtOAc (2×75 mL). The combined organic layers were dried by Na₂SO₄, filtered and concentrated under reduced pressure to afford crude was purified by flash chromatography (eluted in 2% MeOH:DCM) to afford 2-(4-(4-(4-chloroquinolin-6-yl)-3-fluorobenzyl)piperazin-1-yl)ethanol (80 mg, 20.8%). LCMS: 400.3 [M+1].

### Step-3: Synthesis of 2-(4-(4-(4-(benzo[d]thiazol-5-ylamino)quinolin-6-yl)-3-fluorobenzyl)piperazin-1-yl)ethanol

To a solution of 2-(4-(4-(4-chloroquinolin-6-yl)-3-fluorobenzyl)piperazin-1-yl)ethanol (80 mg, 0.20 mmol, 1.0 eq) and benzo[d]thiazol-5-amine (45 mg, 0.30 mmol, 1.5 eq) in EtOH (5 mL) was added TFA (0.1 mL) and the reaction mixture was heated at 120° C. for 16 h. The progress of reaction was monitored by TLC and LCMS. After completion of reaction, reaction mixture was concentrated under reduced pressure to obtain crude which basified with saturated NaHCO₃ Solution (20 mL) and was extracted with DCM (2×50 mL). The combined organic layers were dried by Na₂SO₄, filtered and concentrated under reduced pressure to afford crude was purified by flash chromatography (eluted in 6% MeOH:DCM) to afford 2-(4-(4-(4-(benzo[d]thiazol-5-ylamino)quinolin-6-yl)-3-fluorobenzyl)piperazin-1-yl)ethanol (11 mg, 10.7%), LCMS: 514.4 [M+1]⁺, ¹H NMR (400 MHz, DMSO-d₆) δ ppm 2.08 (s, 2H) 2.44 (br. s., 4H), 2.5 (bs, 4H), 3.50 (d, J=4.77 Hz, 2H) 3.56 (s, 2H) 4.43 (br. s., 1H) 7.05 (d, J=5.25 Hz, 1H) 7.27-7.32 (m, 2H) 7.55 (d, J=8.58 Hz, 1H) 7.68 (t, J=8.11 Hz, 1H) 7.89 (d, J=8.58 Hz, 1H) 7.97 (d, J=9.06 Hz, 1H) 8.04 (s, 1H) 8.20 (d, J=8.58 Hz, 1H) 8.51 (d, J=4.77 Hz, 1H) 8.61 (s, 1H) 9.28 (s, 1H) 9.43 (s, 1H).

### Example 18. Synthesis of N-(6-(4-morpholinophenyl)quinolin-4-yl)benzo[d]thiazol-5-amine (Compound 18)

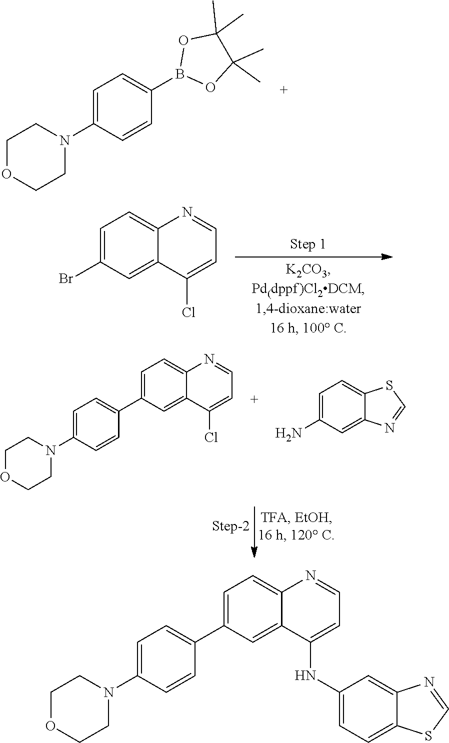 [SMILES: CC1(C)OB(c2ccc(N3CCOCC3)cc2)OC1(C)C ; Clc1ccnc2ccc(Br)cc12 ; Clc1ccnc2ccc(-c3ccc(N4CCOCC4)cc3)cc12 ; Nc1ccc2scnc2c1 ; c1cc(Nc2ccc3scnc3c2)c2cc(-c3ccc(N4CCOCC4)cc3)ccc2n1]

### Step-1: Synthesis of 4-(4-(4-chloroquinolin-6-yl)phenyl)morpholine

A solution of 4-(4-(4,4,5,5-tetramethyl-1,3,2-dioxaborolan-2-yl)phenyl)morpholine (100 mg, 0.345 mmol, 1 eq) in 1,4-dioxane:water (3.5 mL:1.5 mL) were added K2CO3 (95 mg, 0.691 mmol, 2 eq) and 6-bromo-4-chloroquinoline (83 mg, 0.345 mmol, 1 eq). Reaction was purged with nitrogen for 10 min and then added PdCl2dppf·DCM (14 mg, 0.017 mmol, 0.05 eq) was added. The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude was purified by flash chromatography (eluted in 2% MeOH:DCM) to afford 4-(4-(4-chloroquinolin-6-yl)phenyl)morpholine (80 mg, 71.42%). LCMS: 325.3[M+1]⁺.

### Step-2: Synthesis of N-(6-(4-morpholinophenyl)quinolin-4-yl)benzo[d]thiazol-5-amine

To a solution of 4-(4-(4-chloroquinolin-6-yl)phenyl)morpholine (80 mg, 0.246 mmol, 1.0 eq) in ethanol (5 mL) was added benzo[d]thiazol-5-amine (55 mg, 0.369 mmol, 1.5 eq) and TFA (0.08 mL). The reaction was heated at 120° C. for 16 h. After completion of the reaction, the reaction mixture was concentrated under vacuum to obtain crude, which was neutralised by saturated sodium bicarbonate solution (20 mL) and extracted with dichloromethane (2×40 mL), dried over anhydrous sodium sulphate, filtered, and concentrated under vacuum to afford which was purified by flash chromatography (eluted in 5% MeOH:DCM) to afford N-(6-(4-morpholinophenyl)quinolin-4-yl)benzo[d]thiazol-5-amine (3 mg, 2.77%), LCMS: 439.3[M+1]⁺, ¹H NMR (400 MHz, DMSO-d₆) δ ppm 3.20 (br. s., 4H) 3.77 (br. s., 4H) 7.01 (br. s., 1H) 7.10 (d, J=7.63 Hz, 2H) 7.82 (d, J=7.63 Hz, 1H) 7.93 (br. s., 2H) 8.01-8.06 (m, 3H) 8.21 (d, J=8.58 Hz, 1H) 8.44 (br. s., 1H) 8.64 (br. s., 1H) 9.27 (br. s., 1H) 9.43 (br. s., 1H).

### Example 19. Synthesis of N-(3,4-dimethoxyphenyl)-6-(4-(4-methylpiperazin-1-yl)phenyl)quinolin-4-amine (Compound 19)

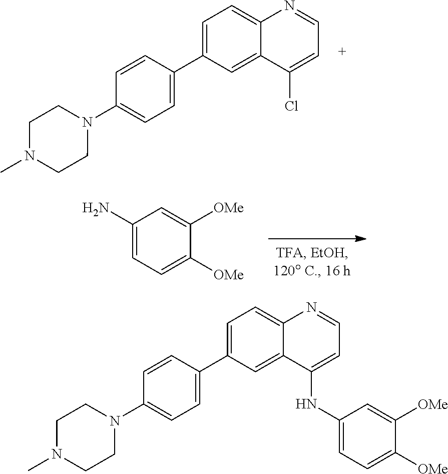 [SMILES: CN1CCN(c2ccc(-c3ccc4nccc(Cl)c4c3)cc2)CC1 ; COc1ccc(N)cc1OC ; COc1ccc(Nc2ccnc3ccc(-c4ccc(N5CCN(C)CC5)cc4)cc23)cc1OC]

To a solution of 4-chloro-6-(4-(4-methylpiperazin-1-yl)phenyl)quinoline (100 mg, 0.295 mmol, 1.0 eq) in ethanol (5 mL) was added 3,4-dimethoxyaniline (68 mg, 0.443 mmol, 1.5 eq) and TFA (0.1 ml). The reaction was heated at 120° C. for 16 h. After completion of reaction, reaction mixture was concentrated under vacuum to obtain crude which was neutralized by saturated sodium bicarbonate solution (20 ml), was extracted with dichloromethane (2×40 ml), dried over anhydrous sodium sulphate, filtered and dried under vacuum to afford crude which was purified by flash chromatography to obtain N-(3,4-dimethoxyphenyl)-6-(4-(4-methylpiperazin-1-yl)phenyl)quinolin-4-amine (6 mg, 4.47%), LCMS: 455[M+1]⁺, ¹H NMR (400 MHz, DMSO-d₆) δ ppm 2.26 (s, 3H) 2.32 (br. s., 4H) 3.23 (br. s., 4H) 3.78 (d, J=7.15 Hz, 6H) 6.73 (d, J=5.72 Hz, 1H) 6.93 (s, 1H) 7.03 (s, 1H) 7.09 (s, 2H) 7.80 (d, J=8.58 Hz, 2H) 7.85 (s, 1H) 7.87 (s, 1H) 8.00 (d, J=9.54 Hz, 1H) 9.08 (br. s., 1H) 8.62 (s, 1H) 8.35 (d, J=5.25 Hz, 1H) 8.00 (d, J=9.54 Hz, 1H).

### Example 20. Synthesis of N-(3,4-dichlorophenyl)-6-(4-(4-methylpiperazin-1-yl)phenyl)quinolin-4-amine (Compound 20)

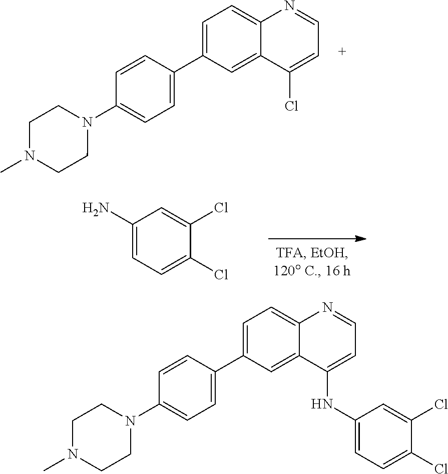 [SMILES: CN1CCN(c2ccc(-c3ccc4nccc(Cl)c4c3)cc2)CC1 ; Nc1ccc(Cl)c(Cl)c1 ; CN1CCN(c2ccc(-c3ccc4nccc(Nc5ccc(Cl)c(Cl)c5)c4c3)cc2)CC1]

To a solution of 4-chloro-6-(4-(4-methylpiperazin-1-yl)phenyl)quinoline (100 mg, 0.295 mmol, 1.0 eq) in ethanol (5 mL) was added 3,4-dichloroaniline (72 mg, 0.443 mmol, 1.5 eq) and TFA (0.1 ml). The reaction was heated at 120° C. for 16 h. After completion of reaction, reaction mixture was neutralized by saturated sodium bicarbonate solution (20 mL) and was extracted with dichloromethane (2×40 mL), dried over anhydrous sodium sulphate, filtered and concentrated under vacuum to afford crude which was purified by flash chromatography to obtain N-(3,4-dichlorophenyl)-6-(4-(4-methylpiperazin-1-yl)phenyl)quinolin-4-amine (33 mg, 24.08%), LCMS: 464[M+1]⁺, ¹H NMR (400 MHz, DMSO-d₆) δ ppm 2.25 (s, 3H), 2.5 (bs, 4H), 3.23 (br. s., 4H) 6.97-7.14 (m, 3H) 7.40 (dd, J=8.82, 2.15 Hz, 1H) 7.60 (d, J=2.38 Hz, 1H) 7.64 (d, J=8.58 Hz, 1H) 7.76 (s, 1H) 7.78 (s, 1H) 7.93 (d, J=8.58 Hz, 1H) 8.02 (d, J=9.06 Hz, 1H) 8.40-8.56 (m, 2H) 9.21 (s, 1H).

### Example 21. Synthesis of N-(6-(3-fluoro-4-((4-methylpiperazin-1-yl)methyl)phenyl)quinolin-4-yl)benzo[d]thiazol-5-amine (Compound 21)

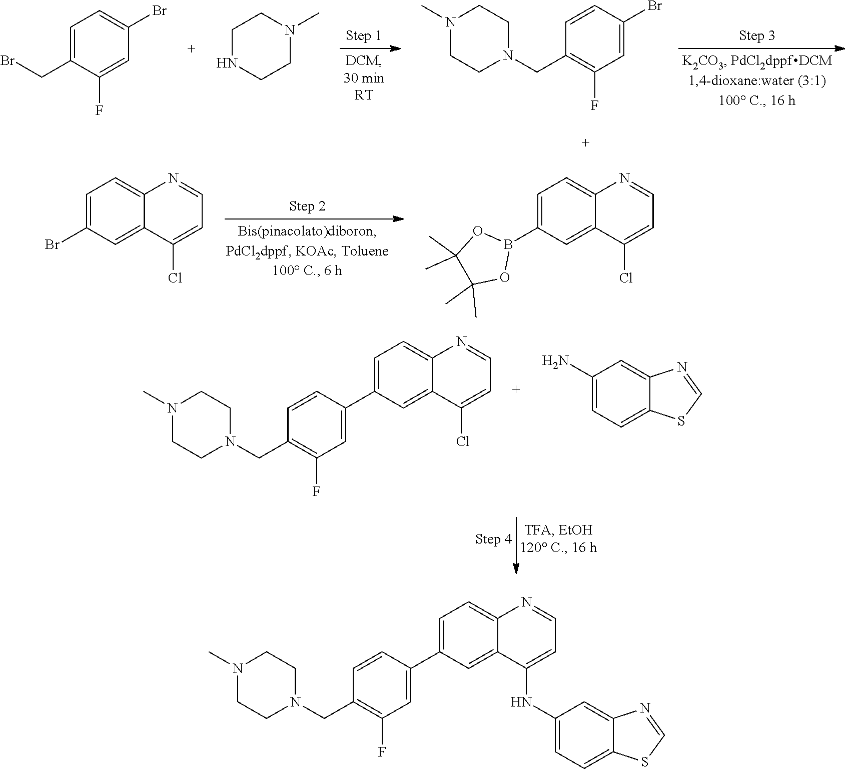

### Step-1: Synthesis of 1-(4-bromo-2-fluorobenzyl)-4-methylpiperazine

A solution of 1-methylpiperazine (373 mg, 3.73 mmol, 2 eq) and 4-bromo-1-(bromomethyl)-2-fluorobenzene (500 mg, 1.86 mmol, 1 eq) in dichloromethane (20 mL) was stirred at RT for 30 min. The progress of reaction was monitored by TLC. After completion of the reaction, the reaction mixture was concentrated under vacuum to afford crude was purified by flash chromatography (eluted in 5% MeOH:DCM) to afford 1-(4-bromo-2-fluorobenzyl)-4-methylpiperazine (200 mg, 37.1%), LCMS: 289.2 [M+2]⁺.

### Step-2: Synthesis of 4-chloro-6-(4,4,5,5-tetramethyl-1,3,2-dioxaborolan-2-yl)quinoline

A solution of 6-bromo-4-chloroquinoline (500 mg, 2.06 mmol, 1.0 eq) in toluene (5 mL) was added potassium acetate (505 mg, 5.15 mmol, 2.5 eq) and Bis(pinacolato)diboron (785 mg, 3.09 mmol, 1.5 eq). The reaction was purged with nitrogen for 5 min and was added PdCl2dppf (75 mg, 0.103 mmol, 0.05 eq). The reaction was heated at 100° C. for 6 h. After completion of the reaction, the reaction mixture was diluted with water (50 mL) and extracted with dichloromethane (100×2 mL), dried over anhydrous sodium sulphate, filtered and concentrated under vacuum to afford 4-chloro-6-(4,4,5,5-tetramethyl-1,3,2-dioxaborolan-2-yl)quinoline (400 mg, 66.4%), LCMS: 439.3[M+1]⁺.

### Step-3: Synthesis of 4-chloro-6-(3-fluoro-4-((4-methylpiperazin-1-yl)methyl)phenyl)quinoline

A solution of 4-chloro-6-(4,4,5,5-tetramethyl-1,3,2-dioxaborolan-2-yl)quinoline (200 mg, 0.696 mmol, 1.0 eq) in dioxane:water (3.5 mL:1.5 mL) was added potassium carbonate (192 mg, 1.39 mmol, 2.0 eq) and 1-(4-bromo-2-fluorobenzyl)-4-methylpiperazine (200 mg, 0.696 mmol, 1.0 eq). The reaction was purged with nitrogen for 5 min and then PdCl2dppf·DCM (28 mg, 0.034 mmol, 0.05 eq). The reaction was heated at 100° C. for 16 h. After completion of the reaction, the reaction mixture was diluted with water (30 mL) and extracted with dichloromethane (75×2 mL), dried over anhydrous sodium sulphate, filtered and dried under vacuum to afford crude which was purified by flash chromatography (eluted in 6% MeOH:DCM) to afford 4-chloro-6-(3-fluoro-4-((4-methylpiperazin-1-yl)methyl)phenyl)quinoline (70 mg, 27.2%), LCMS: 370.3[M+1]⁺.

### Step-4: Synthesis of N-(6-(3-fluoro-4-((4-methylpiperazin-1-yl)methyl)phenyl)quinolin-4-yl)benzo[d]thiazol-5-amine

A solution of 4-chloro-6-(3-fluoro-4-((4-methylpiperazin-1-yl)methyl)phenyl)quinoline (70 mg, 0.189 mmol, 1.0 eq) in ethanol (5 mL) was added benzo[d]thiazol-5-amine (42 mg, 0.283 mmol, 2.0 eq) and TFA (0.1 mL). The reaction was heated at 120° C. for 16 h. After completion of reaction, the reaction mixture was concentrated under vacuum to obtain crude which was neutralized by saturated sodium bicarbonate solution (10 mL) and extracted with dichloromethane (2×30 mL), dried over anhydrous sodium sulphate, filtered and concentrated under vacuum to afford crude was purified by flash chromatography (eluted in 8% MeOH:DCM) to afford N-(6-(3-fluoro-4-((4-methylpiperazin-1-yl)methyl)phenyl)quinolin-4-yl)benzo[d]thiazol-5-amine (30 mg, 32.96%), LCMS: 484.2[M+1]⁺, ¹H NMR (400 MHz, DMSO-d₆) δ ppm 1.23 (s, 3H) 2.33 (br. s., 4H) 2.67 (br. s., 4H) 3.60 (s, 2H) 7.01 (d, J=5.25 Hz, 1H) 7.52-7.60 (m, 2H) 7.72-7.82 (m, 2H) 7.97 (d, J=8.11 Hz, 1H) 8.05-8.13 (m, 2H) 8.23 (d, J=8.58 Hz, 1H) 8.48 (d, J=5.25 Hz, 1H) 8.77 (s, 1H) 9.44 (s, 2H).

### Example 22. Synthesis of N-(6-(2-fluoro-4-(morpholinomethyl)phenyl)quinolin-4-yl)benzo[d]thiazol-5-amine (Compound 22)

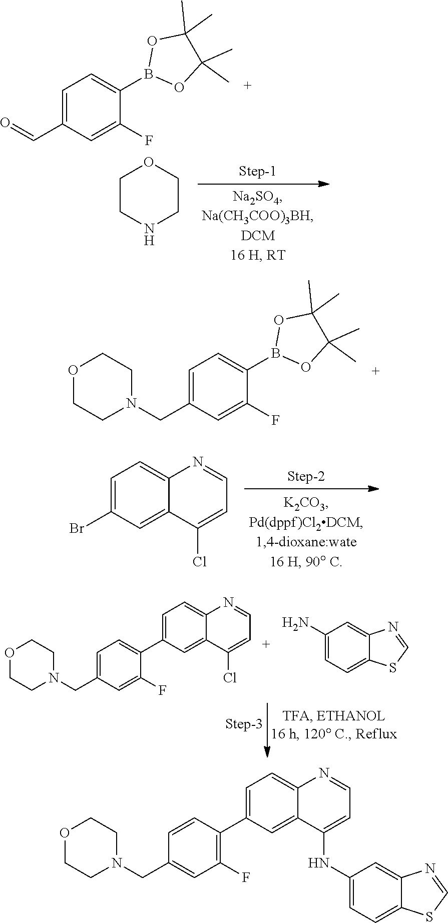

### Step-1: Synthesis of 4-(3-fluoro-4-(4,4,5,5-tetramethyl-1,3,2-dioxaborolan-2-yl)benzyl)morpholine

To a stirred solution of 3-fluoro-4-(4,4,5,5-tetramethyl-1,3,2-dioxaborolan-2-yl)benzaldehyde (400 mg, 1.60 mmol, 1 eq) and morpholine (167.2 mg, 1.91 mmol, 1.2 eq) in DCM (15 mL), was added Na₂SO₄ (455 mg, 3.20 mmol, 2.0 eq). Reaction mixture was stirred at RT for 1 h, was added sodium triacetoxyborohydride (509 mg, 2.4 mmol, 1.5 eq) portion wise at 0° C. After addition, reaction mixture was stirred at RT for 16 h. The progress of reaction was monitored by LCMS. After completion of the reaction, reaction mixture was diluted with water (75 mL) and was extracted with DCM (2×100 mL). The combined organic layers were washed with saturated NaHCO₃ solution (50 mL), Organic layer was dried by Na₂SO₄, filtered and concentrated under reduced pressure to afford 4-(3-fluoro-4-(4,4,5,5-tetramethyl-1,3,2-dioxaborolan-2-yl)benzyl)morpholine (400 mg, 77%), LCMS:240 [Boronic-acid Fragment].

### Step-2: Synthesis of 4-(4-(4-chloroquinolin-6-yl)-3-fluorobenzyl)morpholine

To a stirred solution of 6-bromo-4-chloroquinoline (250 mg, 1.04 mmol, 1.0 eq) and 4-(3-fluoro-4-(4,4,5,5-tetramethyl-1,3,2-dioxaborolan-2-yl)benzyl)morpholine (397.4 mg, 1.23 mmol, 1.2 eq) in 1,4-dioxane:water (6 mL:1.5 mL), was added K₂CO₃ (215.3 mg, 1.56 mmol, 1.5 eq). Reaction mixture was purged with N2 gas for 10 minutes, was added Pd(dppf)Cl2·DCM (42.4 mg, 0.052 mmol, 0.05 eq). After addition, reaction mixture was heated at 90° C. for 16 h. The progress of reaction was monitored by LCMS and TLC. After completion of reaction, reaction mixture was diluted with water (50 mL) and was extracted with EtOAc (2×75 mL). The combined organic layers were dried by Na₂SO₄, filtered and concentrated under reduced pressure to afford crude was purified by flash chromatography (eluted in 2% MeOH:DCM) to afford 4-(4-(4-chloroquinolin-6-yl)-3-fluorobenzyl)morpholine (250 mg, 67%), LCMS: 357.3 [M+1]⁺.

### Step-3: Synthesis of N-(6-(2-fluoro-4-(morpholinomethyl)phenyl)quinolin-4-yl)benzo[d]thiazol-5-amine

To a solution of 4-(4-(4-chloroquinolin-6-yl)-3-fluorobenzyl)morpholine (100 mg, 0.280 mmol, 1.0 eq) and benzo[d]thiazol-5-amine (63.2 mg, 0.420 mmol, 1.5 eq) in EtOH (4 mL) was added TFA (0.08 mL) and the reaction mixture was allowed to reflux at 120° C. for 16 h. The progress of reaction was monitored by TLC and LCMS. After completion of reaction, reaction mixture was concentrated under reduced pressure to obtain crude which was basified with saturated NaHCO₃ (25 mL) and was extracted with DCM (2×35 mL). The combined organic layers were dried by Na2SO4, filtered and concentrated under reduced pressure to afford crude product. Crude product was purified by flash chromatography, eluted in 6% MeOH:DCM to afford N-(6-(2-fluoro-4-(morpholinomethyl)phenyl)quinolin-4-yl)benzo[d]thiazol-5-amine (8.70 mg, 6%), LCMS: 471.3 [M+1]⁺, ¹H NMR (400 MHz, DMSO-d₆) δ ppm 9.43 (s, 1H) 9.27 (br. s., 1H) 8.60 (s, 1H) 8.51 (d, J=5.25 Hz, 1H) 8.20 (d, J=8.58 Hz, 1H) 8.01-8.05 (m, 1H) 7.97 (d, J=9.06 Hz, 1H) 7.89 (d, J=8.58 Hz, 1H) 7.68 (t, J=8.11 Hz, 1H) 7.54-7.58 (m, 1H) 7.33 (s, 1H) 7.30 (br. s., 1H) 7.05 (d, J=5.25 Hz, 1H) 3.60 (t, J=4.29 Hz, 4H) 3.56 (s, 2H) 2.41 (br. s., 4H).

### Example 23. Synthesis of N-(6-(2-fluoro-4-(pyrrolidin-1-ylmethyl)phenyl)quinolin-4-yl)benzo[d]thiazol-5-amine (Compound 23)

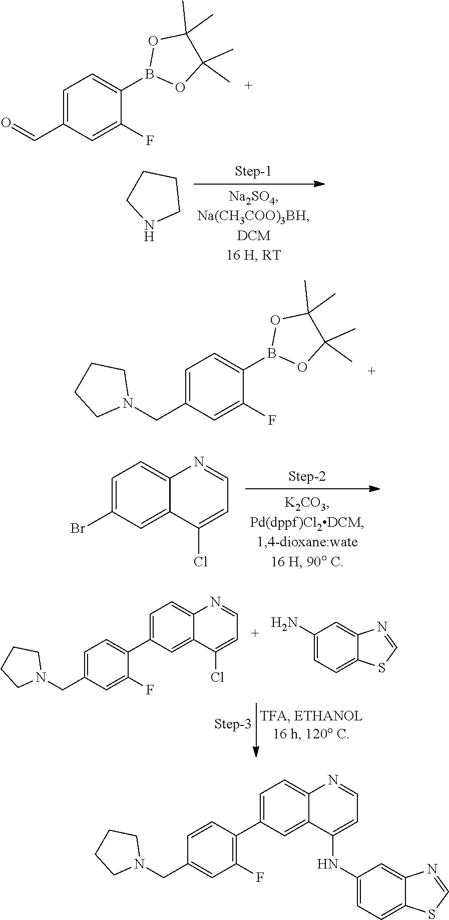

### Step-1: Synthesis of 1-(3-fluoro-4-(4,4,5,5-tetramethyl-1,3,2-dioxaborolan-2-yl)benzyl)pyrrolidine

To a stirred solution of 3-fluoro-4-(4,4,5,5-tetramethyl-1,3,2-dioxaborolan-2-yl)benzaldehyde (400 mg, 1.59 mmol, 1 eq) and pyrrolidine (136.5 mg, 1.908 mmol, 1.2 eq) in DCM (15 mL), we added Na₂SO₄ (452.00 mg, 3.18 mmol, 2.0 eq). Reaction mixture was stirred at RT for 1 h and was added sodium triacetoxyborohydride (505.4 mg, 2.385 mmol, 1.5 eq) portion wise at 0° C. After addition, reaction mixture was stirred at RT for 16 h. The progress of the reaction was monitored by LCMS. After completion of reaction, reaction mixture was diluted with water (75 mL) and was extracted with DCM (2×100 mL). The combined organic layers were washed with saturated NaHCO₃ solution (50 mL). Then, organic layer was dried by Na2SO4, filtered and concentrated under reduced pressure to afford 1-(3-fluoro-4-(4,4,5,5-tetramethyl-1,3,2-dioxaborolan-2-yl)benzyl)pyrrolidine (480 mg, 98.36%). LCMS: 224.3 [Boronic-acid Fragment].

### Step-2: Synthesis of 4-chloro-6-(2-fluoro-4-(pyrrolidin-1-ylmethyl)phenyl)quinoline

To a stirred solution of 6-bromo-4-chloroquinoline (200 mg, 0.83 mmol, 1.0 eq) and 1-(3-fluoro-4-(4,4,5,5-tetramethyl-1,3,2-dioxaborolan-2-yl)benzyl)pyrrolidine (302.05 mg, 0.98 mmol, 1.2 eq) in 1,4-dioxane:water (6 mL:1.5 mL), was added K₂CO₃ (171.12 mg, 1.24 mmol, 1.5 eq). Reaction mixture was purged with N₂ gas for 10 minutes and was added Pd(dppf)Cl2·DCM (33.8 mg, 0.041 mmol, 0.05 eq). After addition, Reaction mixture was stirred at 90° C. for 16 h. The progress of reaction was monitored by LCMS and TLC. After completion of reaction, reaction mixture was diluted with water (50 mL) and was extracted with EtOAc (2×75 mL). The combined organic layers were dried by Na₂SO₄, filtered and concentrated under reduced pressure to afford crude which was purified by flash chromatography (eluted in 2% MeOH:DCM) to afford 4-chloro-6-(2-fluoro-4-(pyrrolidin-1-ylmethyl)phenyl)quinoline (230 mg, 81.2%). LCMS: 341.3 [M+1]⁺.

### Step-3: Synthesis of N-(6-(2-fluoro-4-(pyrrolidin-1-ylmethyl)phenyl)quinolin-4-yl)benzo[d]thiazol-5-amine

To a solution of 4-chloro-6-(2-fluoro-4-(pyrrolidin-1-ylmethyl)phenyl)quinoline (60 mg, 0.17 mmol, 1.0 eq) and benzo[d]thiazol-5-amine (39.6 mg, 0.26 mmol, 1.5 eq) in EtOH (3 mL) was added TFA (0.05 mL) and the reaction mixture was allowed to reflux at 120° C. for 16 h. The progress of reaction was monitored by TLC and LCMS. After completion of reaction, reaction mixture was concentrated under reduced pressure to obtain crude which was basified with saturated NaHCO₃ solution (25 mL) and was extracted with DCM (2×40 mL). The combined organic layers were dried by Na₂SO₄, filtered and concentrated under reduced pressure to afford crude which was purified by flash chromatography (eluted in 6.5% MeOH:DCM) to afford N-(6-(2-fluoro-4-(pyrrolidin-1-ylmethyl)phenyl)quinolin-4-yl)benzo[d]thiazol-5-amine (12.39 mg, 16.25%), LCMS: 455.3 [M+1]⁺, ¹H NMR (400 MHz, DMSO-d₆) δ ppm 9.43 (s, 1H) 9.28 (s, 1H) 8.60 (s, 1H) 8.51 (d, J=5.25 Hz, 1H) 8.20 (d, J=8.58 Hz, 1H) 8.04 (s, 1H) 7.97 (d, J=8.58 Hz, 1H) 7.89 (d, J=8.58 Hz, 1H) 7.68 (t, J=8.34 Hz, 1H) 7.55 (d, J=8.58 Hz, 1H) 7.26-7.38 (m, 2H) 7.05 (d, J=5.25 Hz, 1H) 3.69 (s, 2H) 2.79 (br. s., 4H) 1.74 (br. s., 4H).

### Example 24. Synthesis of N-(6-(6-(4-methylpiperazin-1-yl)pyridin-3-yl)quinolin-4-yl)benzo[d]thiazol-5-amine (Compound 24)

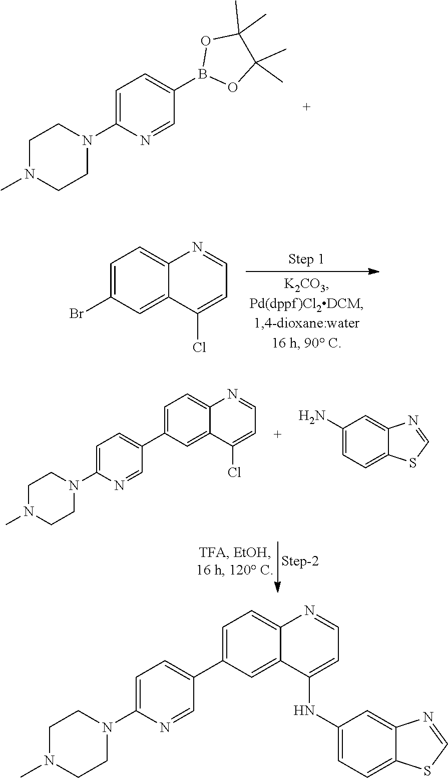

### Step-1: Synthesis of 4-chloro-6-(6-(4-methylpiperazin-1-yl)pyridin-3-yl)quinoline

To a stirred solution of 6-bromo-4-chloroquinoline (250 mg, 1.04 mmol, 1.0 eq) and 1-methyl-4-(5-(4,4,5,5-tetramethyl-1,3,2-dioxaborolan-2-yl)pyridin-2-yl)piperazine (377.5 mg, 1.24 mmol, 1.2 eq) in 1,4-dioxane:water (6 mL:1.5 mL), was added K₂CO₃ (215.3 mg, 1.56 mmol, 1.5 eq). Reaction mixture was purged with N₂ gas for 10 minutes and then was added Pd(dppf)Cl₂·DCM (42.4 mg, 0.052 mmol, 0.05 eq). After addition, reaction mixture stirred at 90° C. for 16 h. The progress of the reaction was monitored by LCMS and TLC. After completion of reaction, reaction mixture was diluted with water (50 mL) and was extracted with EtOAc (2×75 mL). The combined organic layers were dried by Na₂SO₄, filtered and concentrated under reduced pressure to afford crude which was purified by flash chromatography (eluted in 2.5% MeOH:DCM) to afford 4-chloro-6-(6-(4-methylpiperazin-1-yl)pyridin-3-yl)quinoline (220 mg, 63.0%), LCMS: 339.3 [M+1]⁺.

### Step-2: Synthesis of N-(6-(6-(4-methylpiperazin-1-yl)pyridin-3-yl)quinolin-4-yl)benzo[d]thiazol-5-amine

To a solution of 4-chloro-6-(6-(4-methylpiperazin-1-yl)pyridin-3-yl)quinoline (50 mg, 0.148 mmol, 1.0 eq) and benzo[d]thiazol-5-amine (33.3 mg, 0.22 mmol, 1.5 eq) in EtOH (3 mL) was added TFA (0.05 mL) and the reaction mixture was allowed to reflux at 120° C. for 16 h. The progress of reaction was monitored by TLC and LCMS. After completion of the reaction, the reaction mixture was concentrated under reduced pressure to obtain crude, which was basified with a saturated NaHCO3 solution (15 mL) and extracted with DCM (2×25 mL). The combined organic layers were dried by Na2SO4, filtered and concentrated under reduced pressure to afford crude which was purified by flash chromatography (eluted in 7% MeOH:DCM) to afford N-(6-(6-(4-methylpiperazin-1-yl)pyridin-3-yl)quinolin-4-yl)benzo[d]thiazol-5-amine. (20.15 mg, 31.3%), LCMS: 453.3 [M+1]⁺, ¹H NMR (400 MHz, DMSO-d₆) δ ppm 9.44 (s, 1H) 9.31 (br. s., 1H) 8.73 (d, J=2.38 Hz, 1H) 8.68 (s, 1H) 8.45 (d, J=5.25 Hz, 1H) 8.22 (d, J=8.58 Hz, 1H) 8.13 (d, J=2.38 Hz, 1H) 8.03-8.07 (m, 2H) 7.94 (s, 1H) 7.57 (d, J=8.58 Hz, 1H) 7.02 (d, J=5.25 Hz, 2H) 3.60 (br. s., 4H) 2.57 (br. s., 4H) 2.28 (s, 3H).

### Example 25. Synthesis of N-(6-(6-(piperidin-1-yl)pyridin-3-yl)quinolin-4-yl)benzo[d]thiazol-5-amine (Compound 25)

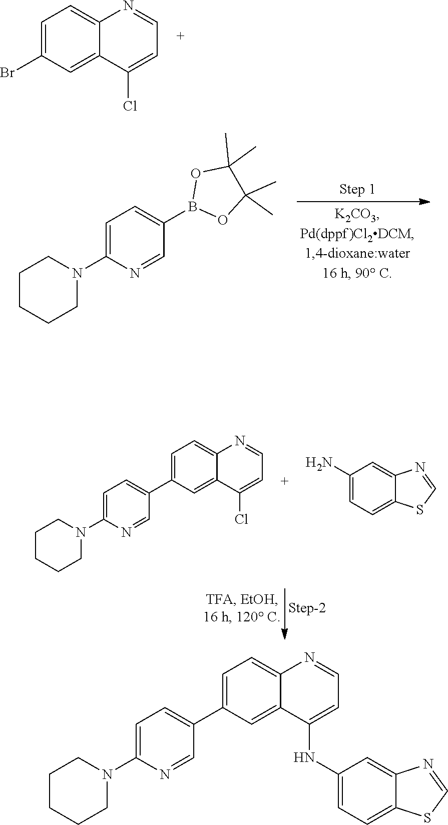 [SMILES: Clc1ccnc2ccc(Br)cc12 ; CC1(C)OB(c2ccc(N3CCCCC3)nc2)OC1(C)C ; Clc1ccnc2ccc(-c3ccc(N4CCCCC4)nc3)cc12 ; Nc1ccc2scnc2c1 ; c1cc(Nc2ccc3scnc3c2)c2cc(-c3ccc(N4CCCCC4)nc3)ccc2n1]

### Step-1: Synthesis of 4-chloro-6-(6-(piperidin-1-yl)pyridin-3-yl)quinoline

To a stirred solution of 6-bromo-4-chloroquinoline (100 mg, 0.412 mmol, 1.0 eq) and 2-(piperidin-1-yl)-5-(4,4,5,5-tetramethyl-1,3,2-dioxaborolan-2-yl)pyridine (142.6 mg, 0.494 mmol, 1.2 eq) in 1,4-dioxane:water (4 mL:1 mL), was added K2CO3 (215.3 mg, 0.618 mmol, 1.5 eq). Reaction was purged with N2 gas for 10 minutes and then was added Pd(dppf)Cl₂·DCM (33.6 mg, 0.041 mmol, 0.05 eq). After addition, reaction mixture was stirred at 90° C. for 16 h. The Progress of reaction was monitored by LCMS and TLC. After completion of reaction, reaction mixture was diluted with water (30 mL) and was extracted with EtOAc (2×50 mL). The combined organic layers were dried by Na₂SO₄, filtered and concentrated under reduced pressure to afford crude which was purified by flash chromatography (eluted in 2.5% MeOH:DCM) to afford 4-chloro-6-(6-(piperidin-1-yl)pyridin-3-yl)quinoline (100 mg, 74.6%), LCMS: 324.3 [M+1]⁺.

### Step-2: Synthesis of N-(6-(6-(piperidin-1-yl)pyridin-3-yl)quinolin-4-yl)benzo[d]thiazol-5-amine

To a solution of 4-chloro-6-(6-(piperidin-1-yl)pyridin-3-yl)quinoline (100 mg, 0.308 mmol, 1.0 eq) and benzo[d]thiazol-5-amine (55.66 mg, 0.370 mmol, 1.5 eq) in EtOH (3 mL) was added TFA (0.08 mL) and the reaction mixture was heated at 120° C. for 16 h. The progress of reaction was monitored by TLC and LCMS. After completion of reaction, reaction mixture was concentrated under reduced pressure to obtain crude which was basified with saturated NaHCO₃ solution (25 mL) and was extracted with DCM (2×50 mL). The combined organic layers were dried by Na₂SO₄, filtered and concentrated under reduced pressure to afford crude was purified by flash chromatography (eluted in 7.5% MeOH:DCM) to afford N-(6-(6-(piperidin-1-yl)pyridin-3-yl)quinolin-4-yl)benzo[d]thiazol-5-amine (119 mg, 88.14%), LCMS: 438.3 [M+1]⁺, ¹H NMR (400 MHz, DMSO-d₆) δ ppm 9.51 (s, 1H) 8.86 (s, 1H) 8.73 (d, J=2.38 Hz, 1H) 8.48 (d, J=6.20 Hz, 1H) 8.34 (d, J=8.58 Hz, 2H) 8.28 (s, 1H) 8.18 (d, J=1.43 Hz, 1H) 8.10 (dd, J=8.82, 2.62 Hz, 1H) 7.99 (d, J=8.58 Hz, 1H) 7.63 (d, J=1.91 Hz, 1H) 7.01 (d, J=9.06 Hz, 1H) 6.95 (s, 1H) 3.63 (d, J=5.72 Hz, 4H) 1.64 (br. s., 2H) 1.57 (br. s., 4H).

### Example 26. Synthesis of N5-(6-(2-fluoro-4-(pyrrolidin-1-ylmethyl)phenyl)quinolin-4-yl)benzo[d]thiazole-2,5-diamine (Compound 26)

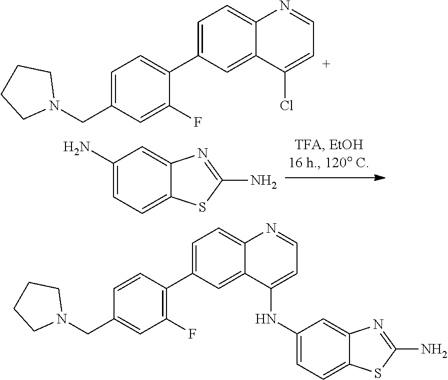

### Synthesis of N5-(6-(2-fluoro-4-(pyrrolidin-1-ylmethyl)phenyl)quinolin-4-yl)benzo[d]thiazole-2,5-diamine

To a solution of 4-chloro-6-(2-fluoro-4-(pyrrolidin-1-ylmethyl)phenyl)quinoline (50 mg, 0.14 mmol, 1.0 eq) and benzo[d]thiazole-2,5-diamine (33.05 mg, 0.22 mmol, 1.5 eq) in EtOH (3 mL) was added TFA (0.05 mL) and the reaction mixture was heated at 120° C. for 16 h. The progress of the reaction was monitored by TLC and LCMS. After completion of reaction, reaction mixture was concentrated under reduced pressure to obtain crude which was basified with saturated NaHCO₃ solution (25 mL) and was extracted with DCM (2×40 mL). The combined organic layers were dried by Na2SO4, filtered and concentrated under reduced pressure to afford crude was purified by flash chromatography (eluted in 12.5% MeOH:DCM) to afford N5-(6-(2-fluoro-4-(pyrrolidin-1-ylmethyl)phenyl)quinolin-4-yl)benzo[d]thiazole-2,5-diamine. (12 mg, 17.6%), LCMS: 470.2 [M+1]⁺, ¹H NMR (400 MHz, DMSO-d₆) δ ppm 9.2 (br. s., 1H) 8.62 (s, 1H) 8.46 (d, J=5.25 Hz, 1H) 7.93-7.98 (m, 1H) 7.84-7.93 (m, 1H) 7.69 (d, J=8.11 Hz, 2H) 7.56 (s, 2H) 7.37 (br. s., 2H) 7.31 (s, 1H) 7.01-7.08 (m, 1H) 6.92 (d, J=5.72 Hz, 1H) 3.69 (s, 2H) 2.76 (br. s., 4H) 1.79 (br. s., 4H).

### Example 27. Synthesis of N-(6-(2-fluoro-4-((4-methyl-1,4-diazepan-1-yl)methyl)phenyl)quinolin-4-yl)benzo[d]thiazol-5-amine (Compound 27)

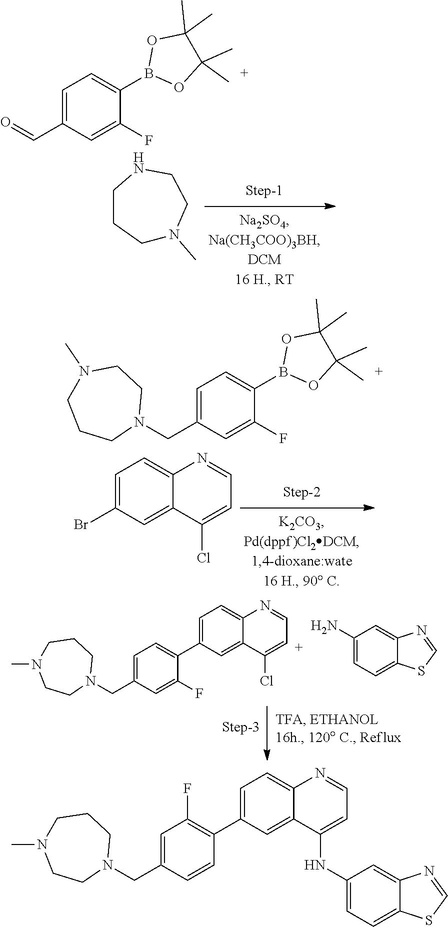

### Step-1: Synthesis of 1-(3-fluoro-4-(4,4,5,5-tetramethyl-1,3,2-dioxaborolan-2-yl)benzyl)-4-methyl-1,4-diazepane

To a stirred solution of 3-fluoro-4-(4,4,5,5-tetramethyl-1,3,2-dioxaborolan-2-yl)benzaldehyde (300 mg, 1.19 mmol, 1 eq) and 1-methyl-1,4-diazepane (150.5 mg, 1.31 mmol, 1.1 eq) in DCM (12 mL), was added Na₂SO₄ (340.9 mg, 2.38 mmol, 2.0 eq). Reaction mixture was stirred at RT for 1 h. Then, was added sodium triacetoxyborohydride (381.3 mg, 1.79 mmol, 1.5 eq) portion wise at 0° C. After addition, reaction mixture was stirred at RT for 16 h. The progress of reaction was monitored by LCMS. After completion of reaction, reaction mixture was diluted with water (75 mL) and was extracted with DCM (2×100 mL). The combined organic layers were washed with saturated NaHCO₃ solution (50 mL). Then, organic layer was dried by Na2SO4, filtered and concentrated under reduced pressure to afford 1-(3-fluoro-4-(4,4,5,5-tetramethyl-1,3,2-dioxaborolan-2-yl)benzyl)-4-methyl-1,4-diazepane (300 mg, 71.9%), LCMS: 267.4 [Boronic-acid Fragment].

### Step-2: Synthesis of 4-chloro-6-(2-fluoro-4-((4-methyl-1,4-diazepan-1-yl)methyl)phenyl)quinoline

To a stirred solution of 6-bromo-4-chloroquinoline (100 mg, 0.41 mmol, 1.0 eq) and 1-(3-fluoro-4-(4,4,5,5-tetramethyl-1,3,2-dioxaborolan-2-yl)benzyl)-4-methyl-1,4-diazepane (172.6 mg, 0.49 mmol, 1.2 eq) in 1,4-dioxane:water (4 mL:1 mL), was added K₂CO₃ (85.36 mg, 0.61 mmol, 1.5 eq). Reaction mixture was purged with N₂ gas for 10 minutes. Then, was added Pd(dppf)Cl2·DCM (33.6 mg, 0.041 mmol, 0.1 eq). After addition, reaction mixture was stirred at 90° C. for 16 h. The progress of the reaction was monitored by LCMS and TLC. After completion of reaction, mixture was diluted with water (25 mL) and was extracted with EtOAc (2×50 mL). The combined organic layers were dried by Na2SO4, filtered and concentrated under reduced pressure to afford which was purified by flash chromatography (eluted in 2% MeOH:DCM) to afford 4-chloro-6-(2-fluoro-4-((4-methyl-1,4-diazepan-1-yl)methyl)phenyl)quinoline. (90 mg, 56.6%). LCMS: 384.3 [M+1]⁺.

### Step-3: Synthesis of N-(6-(2-fluoro-4-((4-methyl-1,4-diazepan-1-yl)methyl)phenyl)quinolin-4-yl)benzo[d]thiazol-5-amine

To a solution of 4-chloro-6-(2-fluoro-4-((4-methyl-1,4-diazepan-1-yl)methyl)phenyl)quinoline (45 mg, 0.117 mmol, 1.0 eq) and benzo[d]thiazol-5-amine (26.4 mg, 0.175 mmol, 1.5 eq) in EtOH (4 mL) was added TFA (0.07 mL) and the reaction mixture was heated at 120° C. for 16 h. The progress of reaction was monitored by TLC and LCMS. After completion of reaction, reaction mixture was concentrated under reduced pressure to obtain crude which was basified with saturated solution of NaHCO₃ (15 mL) and was extracted with DCM (2×30 mL). The combined organic layers were dried by Na₂SO₄, filtered and concentrated under reduced pressure to afford crude was purified by flash chromatography (eluted in 25% MeOH:DCM) to afford N-(6-(2-fluoro-4-((4-methyl-1,4-diazepan-1-yl)methyl)phenyl)quinolin-4-yl)benzo[d]thiazol-5-amine (5.2 mg, 8.9%), LCMS: 498.3 [M+1]⁺, ¹H NMR (400 MHz, DMSO-d₆) δ ppm 9.43 (s, 1H) 9.27 (br. s., 1H) 8.60 (s, 1H) 8.51 (d, J=5.25 Hz, 1H) 8.21 (s, 1H) 8.03 (s, 1H) 7.95-8.00 (m, 1H) 7.86-7.91 (m, 1H) 7.67 (t, J=8.34 Hz, 1H) 7.55 (d, J=8.58 Hz, 1H) 7.28-7.35 (m, 2H) 7.05 (d, J=5.72 Hz, 1H) 3.70 (s, 2H) 2.62-2.70 (m, 8H) 2.30 (s, 3H) 1.72-1.79 (m, 2H).

### Example 28. Synthesis of N-(6-(4-(1-methylpiperidin-4-yl)phenyl)quinolin-4-yl)benzo[d]thiazol-5-amine (Compound 28)

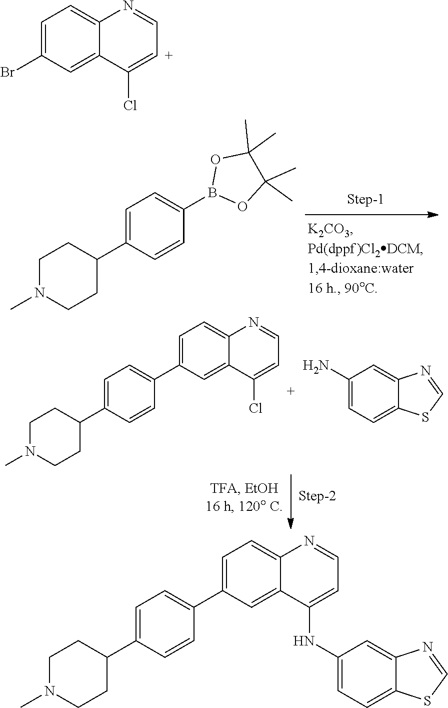

### Step-1: Synthesis of 4-chloro-6-(4-(1-methylpiperidin-4-yl)phenyl)quinoline

To a stirred solution of 6-bromo-4-chloroquinoline (200 mg, 0.824 mmol, 1.0 eq) and 1-methyl-4-(4-(4,4,5,5-tetramethyl-1,3,2-dioxaborolan-2-yl)phenyl)piperidine (298.12 mg, 0.989 mmol, 1.2 eq) in 1,4-dioxane:water (6 mL:1.5 mL), was added K₂CO₃ (170.9 mg, 1.23 mmol, 1.5 eq). Reaction mixture was purged with N2 gas for 10 minutes and then was added Pd(dppf)Cl₂·DCM (33.6 mg, 0.041 mmol, 0.05 eq). After addition, reaction mixture was heated at 90° C. for 16 h. The progress of reaction was monitored by LCMS and TLC. After completion of reaction, reaction mixture was diluted with water (50 mL) and was extracted with EtOAc (2×75 mL). The combined organic layers were dried by Na₂SO₄, filtered and concentrated under reduced pressure to afford crude was purified by flash chromatography (eluted in 2.5% MeOH:DCM) to afford 4-chloro-6-(4-(1-methylpiperidin-4-yl)phenyl)quinoline (200 mg, 71.4%). LCMS: 337.3 [M+1]⁺.

### Step-2: Synthesis of N-(6-(4-(1-methylpiperidin-4-yl)phenyl)quinolin-4-yl)benzo[d]thiazol-5-amine

To a solution of 4-chloro-6-(4-(1-methylpiperidin-4-yl)phenyl)quinoline (100 mg, 0.296 mmol, 1.0 eq) and benzo[d]thiazol-5-amine (66.88 mg, 0.445 mmol, 1.5 eq) in EtOH (3 mL) was added TFA (0.1 mL) and the reaction mixture was heated at 120° C. for 16 h. The progress of reaction was monitored by TLC and LCMS. After completion Of reaction, reaction mixture was concentrated under reduced pressure to obtain crude which was basified with saturated NaHCO₃ (25 mL) and was extracted with DCM (2×50 mL), The combined organic layers were dried by Na₂SO₄, filtered and concentrated under reduced pressure to afford crude was purified by flash chromatography (eluted in 7% MeOH:DCM) to afford N-(6-(4-(1-methylpiperidin-4-yl)phenyl)quinolin-4-yl)benzo[d]thiazol-5-amine (30 mg, 22.55%), LCMS: 451.2 [M+1]⁺, ¹H NMR (400 MHz, DMSO-d₆) δ ppm 9.44 (s, 1H) 9.32 (br. s., 1H) 8.71 (d, J=1.43 Hz, 1H) 8.47 (d, J=5.25 Hz, 1H) 8.21 (d, J=8.58 Hz, 1H) 8.02-8.07 (m, 2H) 7.94-7.98 (m, 1H) 7.86 (m, J=8.11 Hz, 2H) 7.57 (dd, J=8.58, 1.91 Hz, 1H) 7.41 (m, J=8.11 Hz, 2H) 7.02 (d, J=5.25 Hz, 1H) 3.15 (d, J=8.58 Hz, 2H) 2.67 (d, 2H) 2.46 (s, 3H) 2.33 (m, 1H) 1.86-1.95 (m, 2H) 1.82 (d, J=10.97 Hz, 2H).

### Example 29. Synthesis of N-(7-(2-fluoro-4-((4-methylpiperazin-1-yl)methyl)phenyl)quinolin-4-yl)benzo[d]thiazol-5-amine (Compound 29)

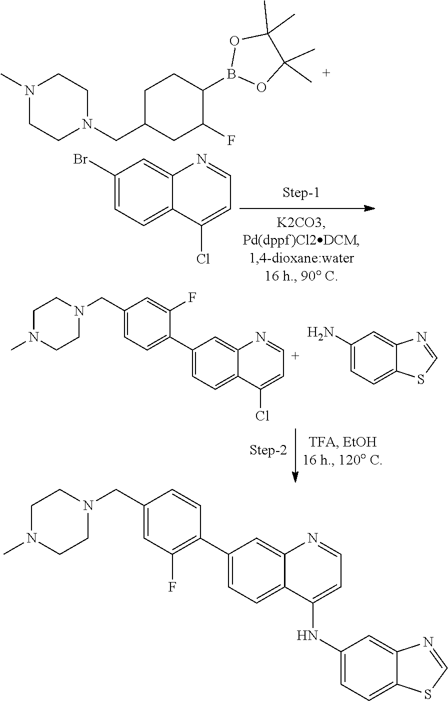

### Step-1: Synthesis of 4-chloro-7-(2-fluoro-4-((4-methylpiperazin-1-yl)methyl)phenyl)quinoline

To a stirred solution of 7-bromo-4-chloroquinoline (145 mg, 0.59 mmol, 1.0 eq) and 1-(3-fluoro-4-(4,4,5,5-tetramethyl-1,3,2-dioxaborolan-2-yl)benzyl)-4-methylpiperazine (200 mg, 0.59 mmol, 1.0 eq) in 1,4-dioxane:water (3.5 ml:1.5 ml), was added K₂CO₃ (165 mg, 1.19 mmol, 2.0 eq). Reaction mixture was purged with N₂ gas for 5 minutes and then Pd(dppf)Cl₂·DCM (24 mg, 0.029 mmol, 0.05 eq) was added. The progress of reaction was monitored by LCMS and TLC. After completion of reaction, reaction mixture was diluted with water (30 mL) and extracted with EtOAc (2×50 mL). The combined organic layers were dried over Na2SO4, filtered and concentrated under reduced pressure to afford crude which was purified by flash chromatography (eluted in 3% MeOH:DCM) to afford 4-chloro-7-(2-fluoro-4-((4-methylpiperazin-1-yl)methyl)phenyl)quinoline (100 mg, 45.4%). LCMS: 370.3 [M+1]⁺.

### Step-2: Synthesis of N-(7-(2-fluoro-4-((4-methylpiperazin-1-yl)methyl)phenyl)quinolin-4-yl)benzo[d]thiazol-5-amine

To a solution of 4-chloro-7-(2-fluoro-4-((4-methylpiperazin-1-yl)methyl)phenyl)quinoline (100 mg, 0.270 mmol, 1.0 eq) and benzo[d]thiazol-5-amine (60 mg, 0.405 mmol, 1.5 eq) in EtOH (5 mL) was added TFA (0.1 mL) and the reaction mixture was heated at 120° C. for 16 h. The progress of reaction was monitored by TLC and LCMS. After completion of the reaction, reaction mixture was concentrated under reduced pressure to obtain crude which was basified with saturated solution of NaHCO₃ (20 mL) and was extracted with DCM (2×50 mL). The combined organic layers were dried over Na2SO4, filtered and concentrated under reduced pressure to afford crude which was purified by flash chromatography (eluted in 6% MeOH:DCM) to afford N-(7-(2-fluoro-4-((4-methylpiperazin-1-yl)methyl)phenyl)quinolin-4-yl)benzo[d]thiazol-5-amine (11 mg, 8.46%), LCMS: 484.3 [M+1]⁺, ¹H NMR (400 MHz, DMSO-d₆) δ ppm 1.23 (s, 3H) 2.33 (s, 4H) 2.53 (s, 4H) 3.59 (br. s., 2H) 7.04 (d, J=5.72 Hz, 1H) 7.32 (s, 2H) 7.57 (s, 1H) 7.70 (s, 1H) 7.75 (s, 1H) 8.21 (d, J=8.58 Hz, 2H) 8.51 (s, 2H) 8.53 (s, 2H) 9.44 (s, 1H).

### Example 30. Synthesis of N-(6-(4-(pyrrolidin-1-yl)phenyl)quinolin-4-yl)benzo[d]thiazol-5-amine (Compound 30)

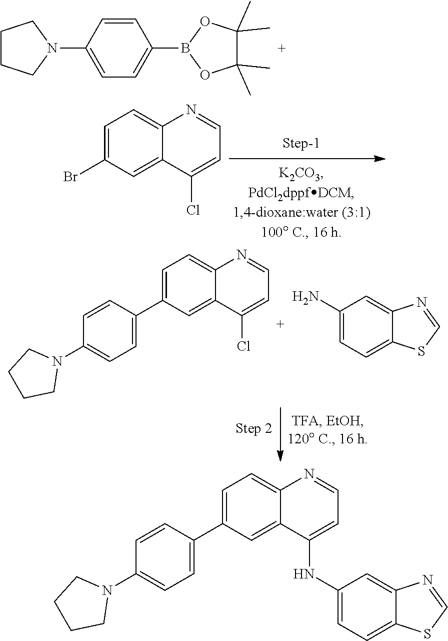

### Step-1: Synthesis of 4-chloro-6-(4-(pyrrolidin-1-yl)phenyl)quinoline

A solution of 1-(4-(4,4,5,5-tetramethyl-1,3,2-dioxaborolan-2-yl)phenyl)pyrrolidine (100 mg, 0.366 mmol, 1 eq) in toluene:water (3.5 mL:1.5 mL) was added K₂CO₃ (101 mg, 0.732 mmol, 2 eq) and 6-bromo-4-chloroquinoline (88 mg, 0.366 mmol, 1 eq). Reaction mixture was purged with nitrogen for 5 min and then was added PdCl₂dppf·DCM (14 mg, 0.001 mmol, 0.05 eq). The reaction mixture was heated at 100° C. for 16 h. The progress of reaction was monitored by TLC. After completion of the reaction, reaction mixture was diluted with water (25 mL) and extracted with ethyl acetate (2×50 mL), dried over anhydrous sodium sulphate, filtered and concentrated under vacuum to afford crude which was purified by flash chromatography (eluted in 2% MeOH:DCM) to afford 4-chloro-6-(4-(pyrrolidin-1-yl)phenyl)quinoline (85 mg, 75.89%). LCMS: 309.2 [M+1]⁺.

### Step-2: Synthesis of N-(6-(4-(pyrrolidin-1-yl)phenyl)quinolin-4-yl)benzo[d]thiazol-5-amine

To a solution of 4-chloro-6-(4-(pyrrolidin-1-yl)phenyl)quinoline (85 mg, 0.275 mmol, 1.0 eq) in ethanol (5 mL) was added benzo[d]thiazol-5-amine (72 mg, 0.412 mmol, 1.5 eq) and TFA (0.1 ml). The reaction was heated at 120° C. for 16 h. After completion of the reaction, reaction mixture was concentrated under vacuum to obtain crude which was neutralized by sodium bicarbonate solution (10 ml) and was extracted with dichloromethane (25×2), dried over anhydrous sodium sulphate, filtered and concentrated under vacuum to afford crude which was purified by flash chromatography (eluted in 5% MeOH:DCM) to afford N-(6-(4-(pyrrolidin-1-yl)phenyl)quinolin-4-yl)benzo[d]thiazol-5-amine (18 mg, 15.5%), LCMS: 423.3[M+1]⁺, ¹H NMR (400 MHz, DMSO-d₆) δ ppm 9.40 (s, 1H) 8.58 (s, 1H) 8.39 (d, J=4.77 Hz, 1H) 8.20 (d, J=8.58 Hz, 2H) 8.01-8.05 (m, 2H) 7.90 (d, J=8.58 Hz, 1H) 7.76 (m, J=8.11 Hz, 2H) 7.56 (d, J=8.58 Hz, 1H) 6.98 (d, J=5.25 Hz, 1H) 6.67 (m, J=8.58 Hz, 2H) 3.28 (br. s., 4H) 1.97 (br. s., 4H).

### Example 31. Synthesis of N-(3,4-dichlorophenyl)-6-(2-fluoro-4-((4-methylpiperazin-1-yl)methyl)phenyl)quinolin-4-amine (Compound 31)

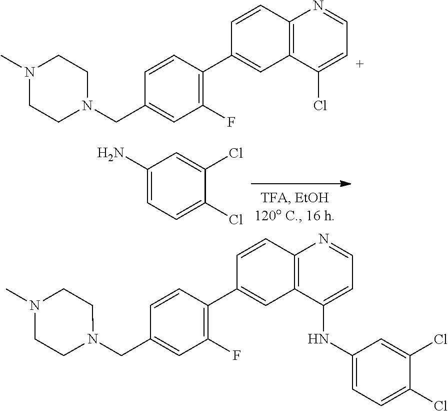

To a solution of 4-chloro-6-(2-fluoro-4-((4-methylpiperazin-1-yl)methyl)phenyl)quinoline (100 mg, 0.270 mmol, 1.0 eq) and 3,4-dichloroaniline (65 mg, 0.405 mmol, 1.5 eq) in EtOH (5 mL) was added TFA (0.1 mL) and the reaction mixture was heated at 120° C. for 16 h. The progress of reaction was monitored by TLC and LCMS. After completion of the reaction, reaction mixture was concentrated under reduced pressure to obtain crude which was basified with saturated solution of NaHCO₃ (20 mL) and was extracted with DCM (2×50 mL). The combined organic layers were dried by Na₂SO₄, filtered and concentrated under reduced pressure to afford crude was purified by flash chromatography (eluted in 6% MeOH:DCM) to afford N-(3,4-dichlorophenyl)-6-(2-fluoro-4-((4-methylpiperazin-1-yl)methyl)phenyl)quinolin-4-amine (3 mg, 3.22%), LCMS: 495.2[M+1], ¹H NMR (400 MHz, DMSO-d₆) δ ppm 2.18 (br. s., 3H) 2.32 (br. s., 4H) 2.41 (br. s., 4H) 3.55 (s, 2H) 7.14 (d, J=5.25 Hz, 1H) 7.27-7.32 (m, 2H) 7.39 (d, J=9.06 Hz, 1H) 7.59 (d, J=1.91 Hz, 1H) 7.61-7.68 (m, 2H) 7.89 (d, J=8.58 Hz, 1H) 7.98 (d, J=9.06 Hz, 1H) 8.49 (s, 1H) 8.58 (d, J=4.77 Hz, 1H) 9.21 (s, 1H).

### Example 32. Synthesis of N-(3,4-dimethoxyphenyl)-6-(2-fluoro-4-((4-methylpiperazin-1-yl)methyl)phenyl)quinolin-4-amine (Compound 32)

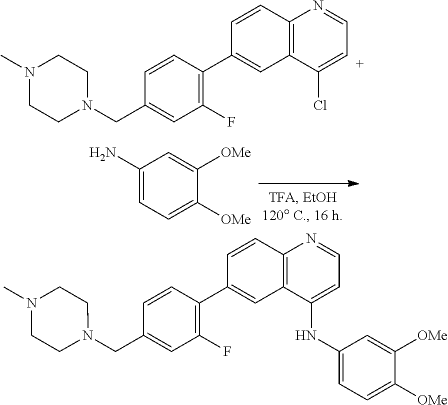

To a solution of 4-chloro-6-(2-fluoro-4-((4-methylpiperazin-1-yl)methyl)phenyl)quinoline (50 mg, 0.135 mmol, 1.0 eq) and 3,4-dimethoxyaniline (31 mg, 0.202 mmol, 1.5 eq) in EtOH (5 mL) was added TFA (0.05 mL) and the reaction mixture was heated at 120° C. for 16 h. The progress of reaction was monitored by TLC and LCMS. After completion of the reaction, reaction mixture was concentrated under reduced pressure to obtain crude was basified with saturated solution of NaHCO₃ (15 mL) and was extracted with DCM (2×30 mL). The combined organic layers were dried by Na₂SO₄, filtered and concentrated under reduced pressure to afford which was purified by flash chromatography (eluted in 5% MeOH:DCM) to afford N-(3,4-dimethoxyphenyl)-6-(2-fluoro-4-((4-methylpiperazin-1-yl)methyl)phenyl)quinolin-4-amine (17 mg, 26.1%), LCMS: 487.3[M+1], ¹H NMR (400 MHz, DMSO-d₆) δ ppm 2.16 (s, 3H) 2.33 (br. s., 4H) 2.49 (br. s., 4H) 3.54 (s, 2H) 3.77 (d, J=8.11 Hz, 6H) 6.80 (d, J=5.25 Hz, 1H) 6.90 (d, J=6.20 Hz, 1H) 6.95 (d, J=1.91 Hz, 1H) 7.02 (d, J=8.58 Hz, 1H) 7.30 (d, J=6.20 Hz, 1H) 7.64-7.69 (m, 1H) 7.84 (m, J=8.58 Hz, 1H) 7.91 (m, J=8.58 Hz, 1H) 8.24 (s, 1H) 8.42 (d, J=5.25 Hz, 1H) 8.56 (s, 1H) 8.95 (br. s., 1H).

### Example 33. Synthesis of N-(4-chloro-3-fluorophenyl)-6-(2-fluoro-4-((4-methylpiperazin-1-yl)methyl)phenyl)quinolin-4-amine (Compound 33)

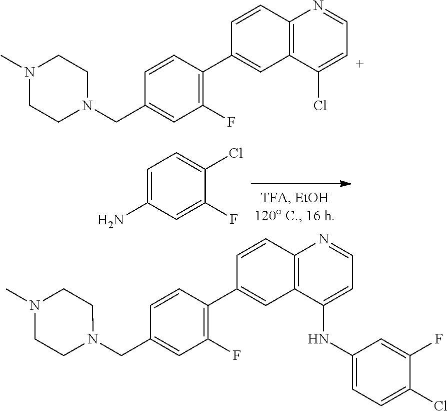

To a solution of 4-chloro-6-(2-fluoro-4-((4-methylpiperazin-1-yl)methyl)phenyl)quinoline (50 mg, 0.135 mmol, 1.0 eq) and 4-chloro-3-fluoroaniline (29 mg, 0.202 mmol, 1.5 eq) in EtOH (5 mL) was added TFA (0.05 mL) and the reaction mixture was heated at 120° C. for 16 h. The progress of reaction was monitored by TLC and LCMS. After completion of the reaction, reaction mixture was concentrated under reduced pressure to obtain crude which was basified with saturated solution of NaHCO₃ (15 mL) and was extracted with DCM (2×30 mL). the combined organic layers were dried over Na₂SO₄, filtered and concentrated under reduced pressure to afford crude which was purified by flash chromatography (eluted in 6% MeOH:DCM) to afford N-(4-chloro-3-fluorophenyl)-6-(2-fluoro-4-((4-methylpiperazin-1-yl)methyl)phenyl)quinolin-4-amine (7 mg, 10.9%), LCMS: 479.3[M+1], ¹H NMR (400 MHz, CDCl3) δ ppm 2.30 (s, 3H) 2.52 (br. s., 8H) 3.57 (s, 2H) 6.98-7.05 (m, 2H) 7.09 (d, J=4.77 Hz, 1H) 7.14 (br. s., 1H) 7.21 (br. s., 3H) 7.39-7.45 (m, 1H) 7.48 (t, J=7.87 Hz, 1H) 7.89 (d, J=8.58 Hz, 1H) 8.05 (s, 1H) 8.13 (d, J=8.58 Hz, 1H) 8.65 (d, J=5.25 Hz, 1H).

### Example 34. Synthesis of (4-(4-(benzo[d]thiazol-5-ylamino)quinolin-7-yl)phenyl)(4-methylpiperazin-1-yl)methanone (Compound 34)

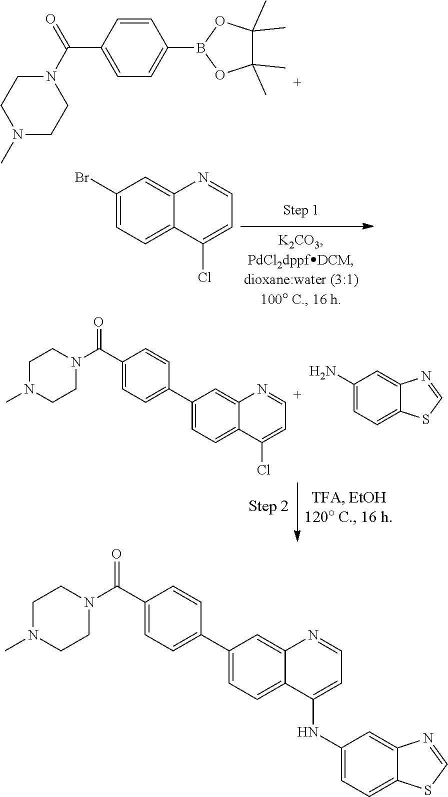

### Step-1: Synthesis of (4-(4-chloroquinolin-7-yl)phenyl)(4-methylpiperazin-1-yl)methanone

To a solution of (4-methylpiperazin-1-yl)(4-(4,4,5,5-tetramethyl-1,3,2-dioxaborolan-2-yl)phenyl)methanone (250 mg, 0.757 mmol, 1 eq) in 1,4-dioxane:water (3.5 mL:1.5 mL) was added K₂CO₃ (209 mg, 1.51 mmol, 2 eq) and 7-bromo-4-chloroquinoline (183 mg, 0.757 mmol, 1 eq). Reaction mixture was purged with nitrogen for 10 min and then was added PdCl2dppf·DCM (30 mg, 0.037 mmol, 0.05 eq). The reaction was heated at 100° C. for 16 h. The progress of reaction was monitored by TLC. After completion of the reaction, reaction mixture was diluted with water (40 mL) and was extracted with ethyl acetate (2×80 mL), dried over anhydrous sodium sulphate, filtered and concentrated under vacuum to afford crude which was purified by flash chromatography to afford (4-(4-chloroquinolin-7-yl)phenyl)(4-methylpiperazin-1-yl)methanone (80 mg, 28.9%). LCMS: 366.3[M+1]⁺.

### Step-2: Synthesis of (4-(4-(benzo[d]thiazol-5-ylamino)quinolin-7-yl)phenyl)(4-methylpiperazin-1-yl)methanone

To a solution of (4-(4-chloroquinolin-7-yl)phenyl)(4-methylpiperazin-1-yl)methanone (80 mg, 0.219 mmol, 1.0 eq) in ethanol (5 mL) was added benzo[d]thiazol-5-amine (49 mg, 0.328 mmol, 1.5 eq) and TFA (0.1 mL). The reaction mixture was heated at 120° C. for 16 h. After completion of the reaction, reaction mixture was concentrated under vacuum to obtain crude which was neutralized by saturated sodium bicarbonate solution (20 ml) and was extracted with dichloromethane (2×40 mL), dried over anhydrous sodium sulphate, filtered and concentrated under vacuum to afford crude which was purified by flash chromatography to afford (4-(4-(benzo[d]thiazol-5-ylamino)quinolin-7-yl)phenyl)(4-methylpiperazin-1-yl)methanone (39 mg, 37.5%), LCMS: 480.3[M+1]⁺, ¹H NMR (400 MHz, DMSO-d₆) δ ppm 9.57 (br. s., 1H) 8.59 (d, J=8.58 Hz, 1H) 8.53 (s, 1H) 8.24 (d, J=8.58 Hz, 1H) 8.21 (s, 2H) 8.08 (s, 1H) 7.97 (d, J=8.11 Hz, 3H) 7.58 (s, 3H) 7.01 (d, J=5.25 Hz, 1H) 3.45 (br. s., 4H) 2.49 (br. s., 4H) 2.32 (br. s., 3H).

### Example 35. Synthesis of (4-(4-(benzo[d]thiazol-5-ylamino)quinolin-6-yl)phenyl)(4-methylpiperazin-1-yl)methanone (Compound 35)

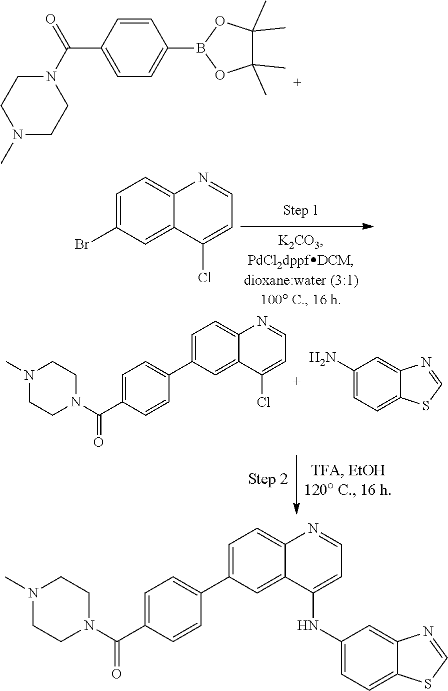

### Step-1: Synthesis of (4-(4-chloroquinolin-6-yl)phenyl)(4-methylpiperazin-1-yl)methanone

A solution of (4-methylpiperazin-1-yl)(4-(4,4,5,5-tetramethyl-1,3,2-dioxaborolan-2-yl)phenyl)methanone (250 mg, 0.757 mmol, 1 eq) in 1,4-dioxane:water (3.5 mL:1.5 mL) was added K₂CO₃ (209 mg, 1.51 mmol, 2 eq) and 6-bromo-4-chloroquinoline (183 mg, 0.757 mmol, 1 eq). Reaction mixture was purged with nitrogen for 10 min and was added PdCl₂dppf·DCM (30 mg, 0.037 mmol, 0.05 eq). The reaction was heated at 100° C. for 16 h. The progress of reaction was monitored by TLC. After completion of the reaction, reaction mixture was diluted with water (40 mL) and was extracted with ethyl acetate (2×80 mL), dried over anhydrous sodium sulphate, filtered and concentrated under vacuum to afford crude which was purified by flash chromatography to obtain (4-(4-chloroquinolin-6-yl)phenyl)(4-methylpiperazin-1-yl)methanone (80 mg, 28.9%), LCMS: 366.3[M+1]⁺.

### Step-2: Synthesis of (4-(4-(benzo[d]thiazol-5-ylamino)quinolin-6-yl)phenyl)(4-methylpiperazin-1-yl)methanone

To a solution of (4-(4-chloroquinolin-6-yl)phenyl)(4-methylpiperazin-1-yl)methanone (80 mg, 0.219 mmol, 1.0 eq) in ethanol (5 mL) was added benzo[d]thiazol-5-amine (49 mg, 0.328 mmol, 1.5 eq) and TFA (0.1 ml). The reaction was heated at 120° C. for 16 h. After completion of the reaction, reaction mixture was concentrated under vacuum to obtain crude which was neutralized by saturated sodium bicarbonate solution (20 ml) and was extracted with dichloromethane (2×40 mL), dried over anhydrous sodium sulphate, filtered and concentrated under vacuum to afford crude which was purified by flash chromatography to afford (4-(4-(benzo[d]thiazol-5-ylamino)quinolin-6-yl)phenyl)(4-methylpiperazin-1-yl)methanone (71 mg, 68.2%), LCMS: 480.4[M+1]⁺, ¹H NMR (400 MHz, DMSO-d₆) δ ppm 9.44 (s, 1H) 9.38 (br. s., 1H) 8.79 (s, 1H) 8.49 (d, J=5.25 Hz, 1H) 8.23 (d, J=8.58 Hz, 1H) 8.05-8.12 (m, 2H) 7.99 (d, J=7.63 Hz, 3H) 7.53-7.59 (m, 3H) 7.03 (d, J=5.25 Hz, 1H) 2.33 (br. s., 4H) 3.63 (br. s., 4H) 2.22 (s, 3H).

### Example 36. Synthesis of N-(6-(4-((4-(tert-butyl)piperazin-1-yl)methyl)-2-fluorophenyl)quinolin-4-yl)benzo[d]thiazol-5-amine (Compound 36)

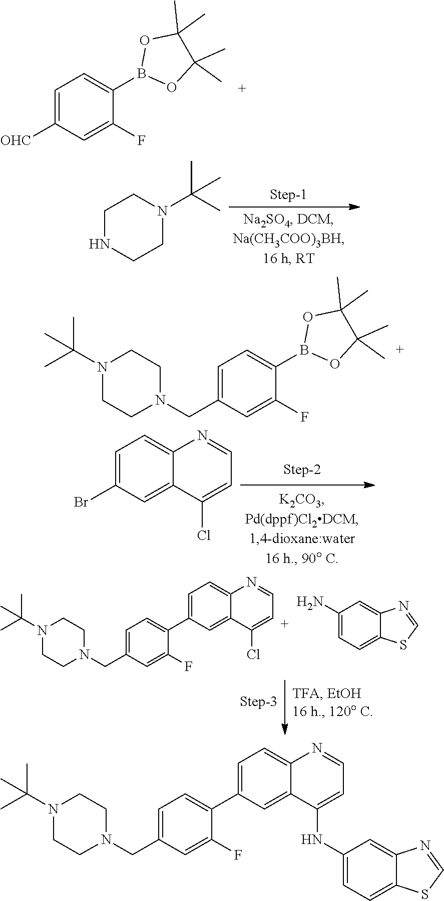

### Step-1: Synthesis of 1-(tert-butyl)-4-(3-fluoro-4-(4,4,5,5-tetramethyl-1,3,2-dioxaborolan-2-yl)benzyl)piperazine

To a stirred solution of 3-fluoro-4-(4,4,5,5-tetramethyl-1,3,2-dioxaborolan-2-yl)benzaldehyde (300 mg, 1.20 mmol, 1 eq) and 1-(tert-butyl)piperazine (255 mg, 1.80 mmol, 1.5 eq) in DCM (15 mL) was added Na₂SO₄ (340 mg, 2.40 mmol, 2.0 eq). Reaction mixture was stirred at RT for 1 h and was added sodium triacetoxyborohydride (506 mg, 1.80 mmol, 1.5 eq) portion wise at 0° C. After addition, reaction mixture was stirred at RT for 16 h. The progress of reaction was monitored by LCMS. After completion of the reaction, reaction mixture was diluted with water (50 mL) and extracted with DCM (2×80 mL). The combined organic layers were washed with saturated solution of NaHCO₃ (50 mL), Organic layer was dried by Na2SO4, filtered and concentrated under reduced pressure to afford 1-(tert-butyl)-4-(3-fluoro-4-(4,4,5,5-tetramethyl-1,3,2-dioxaborolan-2-yl)benzyl)piperazine (370 mg, 82.03%). LCMS: 295.5 [Boronic-acid Fragment].

### Step-2: Synthesis of 6-(4-((4-(tert-butyl)piperazin-1-yl)methyl)-2-fluorophenyl)-4-chloroquinoline

To a stirred solution of 6-bromo-4-chloroquinoline (128 mg, 0.53 mmol, 1.0 eq) and 1-(tert-butyl)-4-(3-fluoro-4-(4,4,5,5-tetramethyl-1,3,2-dioxaborolan-2-yl)benzyl)piperazine (200 mg, 0.53 mmol, 1.0 eq) in 1,4-dioxane:water (3.5 mL:1.5 mL) was added K₂CO₃ (146 mg, 1.06 mmol, 2.0 eq). Reaction mixture was purged with N₂ gas for 10 minutes and was added Pd(dppf)Cl₂·DCM (21 mg, 0.026 mmol, 0.05 eq). The progress of reaction was monitored by LCMS and TLC. After completion of the reaction, reaction mixture was diluted with water (40 mL) and was extracted with EtOAc (2×75 mL). The combined organic layers were dried by Na2SO4, filtered and concentrated under reduced pressure to afford crude which was purified by flash chromatography (eluted in 3% MeOH:DCM) to afford 6-(4-((4-(tert-butyl)piperazin-1-yl)methyl)-2-fluorophenyl)-4-chloroquinoline (80 mg, 36.6%). LCMS: 412.4 [M+1]⁺.

### Step-3: Synthesis of N-(6-(4-((4-(tert-butyl)piperazin-1-yl)methyl)-2-fluorophenyl)quinolin-4-yl)benzo[d]thiazol-5-amine

To a solution of 6-(4-((4-(tert-butyl)piperazin-1-yl)methyl)-2-fluorophenyl)-4-chloroquinoline (80 mg, 0.194 mmol, 1.0 eq) and benzo[d]thiazol-5-amine (43 mg, 0.291 mmol, 1.5 eq) in EtOH (5 mL) was added TFA (0.1 mL). Reaction mixture was heated at 120° C. for 16 h. The progress of reaction was monitored by TLC and LCMS. After completion of the reaction, reaction mixture was concentrated under reduced pressure to obtain crude which was basified with saturated solution of NaHCO₃ (20 mL) and was extracted with DCM (2×40 mL).The combined organic layers were dried by Na2SO4, filtered and concentrated under reduced pressure to afford crude compound which was purified by flash chromatography (eluted in 6% MeOH:DCM) to afford N-(6-(4-((4-(tert-butyl)piperazin-1-yl)methyl)-2-fluorophenyl)quinolin-4-yl)benzo[d]thiazol-5-amine (20 mg, 19.6%), LCMS: 526.3 [M+1]⁺, ¹H NMR (400 MHz, DMSO-d₆) δ ppm 9.43 (s, 1H) 9.31 (br. s., 1H) 8.61 (s, 1H) 8.51 (d, J=5.25 Hz, 1H) 8.21 (d, J=8.58 Hz, 1H) 8.04 (s, 1H) 7.98 (d, J=8.58 Hz, 1H) 7.90 (d, J=8.11 Hz, 1H) 7.65-7.73 (m, 1H) 7.55 (d, J=8.58 Hz, 1H) 7.32 (d, J=7.15 Hz, 2 H) 7.05 (d, J=5.25 Hz, 1H) 3.50 (br. s., 2H) 3.32 (br. s., 4H) 2.49 (s, 4H) 1.23 (br. s., 9H).

### Example 37. Synthesis of N-(7-(4-((4-(tert-butyl)piperazin-1-yl)methyl)-2-fluorophenyl)quinolin-4-yl)benzo[d]thiazol-5-amine (Compound 37)

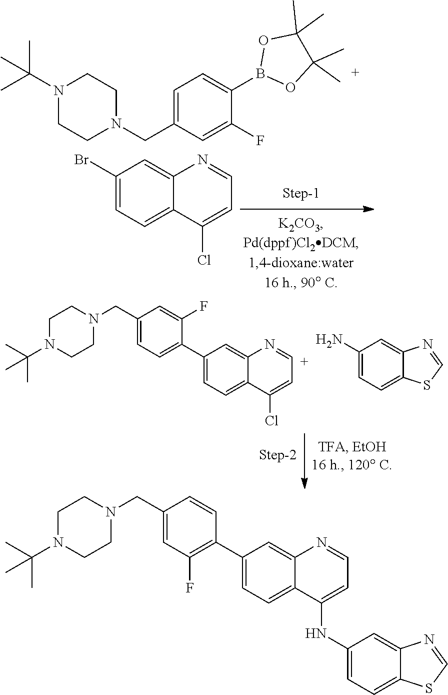

### Step-1: Synthesis of 7-(4-((4-(tert-butyl)piperazin-1-yl)methyl)-2-fluorophenyl)-4-chloroquinoline

To a stirred solution of 7-bromo-4-chloroquinoline (64 mg, 0.265 mmol, 1.0 eq) and 1-(tert-butyl)-4-(3-fluoro-4-(4,4,5,5-tetramethyl-1,3,2-dioxaborolan-2-yl)benzyl)piperazine (100 mg, 0.265 mmol, 1.0 eq) in 1,4-dioxane:water (3.5 mL:1.5 mL), was added K₂CO₃ (73 mg, 0.531 mmol, 2.0 eq). Reaction mixture was purged with N₂ gas for 10 minutes and was added Pd(dppf)Cl₂·DCM (10 mg, 0.013 mmol, 0.05 eq). The progress of reaction was monitored by LCMS and TLC. After completion of the reaction, reaction mixture was diluted with water (20 mL) and was extracted with EtOAc (2×50 mL). The combined organic layers were dried over Na₂SO₄, filtered and concentrated under reduced pressure to afford crude which was purified by flash chromatography (eluted in 3% MeOH:DCM) to afford 7-(4-((4-(tert-butyl)piperazin-1-yl)methyl)-2-fluorophenyl)-4-chloroquinoline (60 mg, 55.04%). LCMS: 412.3 [M+1]⁺.

### Step-2: Synthesis of N-(7-(4-((4-(tert-butyl)piperazin-1-yl)methyl)-2-fluorophenyl)quinolin-4-yl)benzo[d]thiazol-5-amine

To a solution of 7-(4-((4-(tert-butyl)piperazin-1-yl)methyl)-2-fluorophenyl)-4-chloroquinoline (60 mg, 0.145 mmol, 1.0 eq) and benzo[d]thiazol-5-amine (32 mg, 0.218 mmol, 1.5 eq) in EtOH (5 mL) was added TFA (0.05 mL). The reaction mixture was heated at 120° C. for 16 h. The progress of reaction was monitored by TLC and LCMS. After completion of the reaction, reaction mixture was concentrated under reduced pressure to obtain crude which was basified with saturated solution of NaHCO₃ (10 mL) and was extracted with DCM (2×40 mL). The combined organic layers were dried by Na₂SO₄, filtered and concentrated under reduced pressure to afford crude which was purified by flash chromatography (eluted in 7% MeOH:DCM) to afford N-(7-(4-((4-(tert-butyl)piperazin-1-yl)methyl)-2-fluorophenyl)quinolin-4-yl)benzo[d]thiazol-5-amine (8 mg, 10.5%), LCMS: 526.3 [M+1]⁺, ¹H NMR (400 MHz, DMSO-d₆) δ ppm 9.43 (s, 1H) 9.26 (s, 1H) 8.48-8.54 (m, 2H) 8.20 (d, J=8.58 Hz, 1H) 8.05 (br. s., 2H) 7.76 (d, J=8.58 Hz, 1H) 7.67-7.72 (m, 1H) 7.56 (d, J=9.06 Hz, 1H) 7.30 (d, J=8.58 Hz, 2H) 7.04 (d, J=5.25 Hz, 1H) 3.55 (br. s., 2H) 3.38 (br. s., 4H) 2.33 (br. s., 4H) 1.02 (br. s., 9H).

### Example 38. Synthesis of tert-butyl 4-(4-(4-(benzo[d]thiazol-5-ylamino)quinolin-6-yl)-3-fluorobenzyl)piperazine-1-carboxylate (Compound 38)

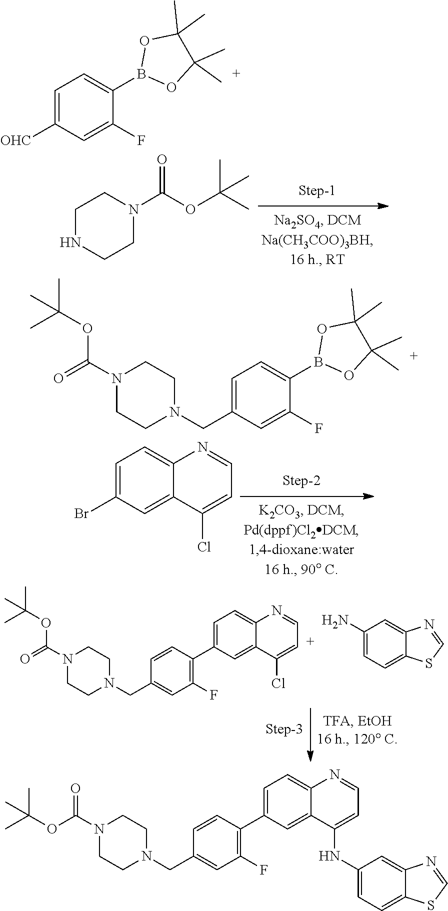

### Step-1: Synthesis of tert-butyl 4-(3-fluoro-4-(4,4,5,5-tetramethyl-1,3,2-dioxaborolan-2-yl)benzyl)piperazine-1-carboxylate

To a stirred solution of 3-fluoro-4-(4,4,5,5-tetramethyl-1,3,2-dioxaborolan-2-yl)benzaldehyde (150 mg, 0.599 mmol, 1 eq) and tert-butyl piperazine-1-carboxylate (122.9 mg, 0.659 mmol, 1.1 eq) in DCM (7 mL) was added Na₂SO₄ (170.3 mg, 1.198 mmol, 2.0 eq). Reaction mixture was stirred at RT for 1 h and was added Sodium triacetoxyborohydride (190.5 mg, 0.898 mmol, 1.5 eq.) portion wise at 0° C. Resulting reaction was stirred at RT for 16 h. The progress of reaction was monitored by LCMS. After completion of the reaction, reaction mixture was diluted with water (50 mL) and extracted with DCM (2×75 mL). The combined organic layers were washed with saturated NaHCO₃ (50 mL) solution, organic layer was dried by Na₂SO₄, filtered and concentrated under reduced pressure to afford tert-butyl 4-(3-fluoro-4-(4,4,5,5-tetramethyl-1,3,2-dioxaborolan-2-yl)benzyl)piperazine-1-carboxylate (215 mg, 85.65%). LCMS: 339.4 [Boronic-acid Fragment].

### Step-2: Synthesis of tert-butyl 4-(4-(4-chloroquinolin-6-yl)-3-fluorobenzyl)piperazine-1-carboxylate

To a stirred solution of 6-bromo-4-chloroquinoline (200 mg, 0.824 mmol, 1.0 eq) and tert-butyl 4-(3-fluoro-4-(4,4,5,5-tetramethyl-1,3,2-dioxaborolan-2-yl)benzyl)piperazine-1-carboxylate (415.6 mg, 0.989 mmol, 1.2 eq) in 1,4-dioxane:water (6 mL:1.5 mL) was added K₂CO₃ (171.9 mg, 1.25 mmol, 1.5 eq). Reaction mixture was purged with N₂ gas for 10 minutes and was added Pd(dppf)Cl₂·DCM (33.9 mg, 0.042 mmol, 0.05 eq). Reaction mixture was stirred at 90° C. for 16 h. The Progress of reaction was monitored by LCMS and TLC. After completion of the reaction, reaction mixture was diluted with water (75 mL) and was extracted with EtOAc (2×100 mL). The combined organic layers were dried by Na₂SO₄, filtered and concentrated under reduced pressure to afford crude product which was purified by flash chromatography (eluted in 2% MeOH:DCM) to afford tert-butyl 4-(4-(4-chloroquinolin-6-yl)-3-fluorobenzyl)piperazine-1-carboxylate (211 mg, 55.96%). LCMS: 456.2 [M+1]⁺.

### Step-3: Synthesis of tert-butyl 4-(4-(4-(benzo[d]thiazol-5-ylamino)quinolin-6-yl)-3-fluorobenzyl)piperazine-1-carboxylate

To a solution of afford tert-butyl 4-(4-(4-chloroquinolin-6-yl)-3-fluorobenzyl)piperazine-1-carboxylate (25 mg, 0.054 mmol, 1.0 eq) and benzo[d]thiazol-5-amine (8.10 mg, 0.054 mmol, 1.0 eq) in EtOH (2 mL) was added TFA (0.05 mL) and the reaction mixture was heated at 120° C. for 16 h. The progress of the reaction was monitored by TLC and LCMS. After completion of the reaction, reaction mixture was concentrated under reduced pressure to obtain crude which was basified with saturated solution of NaHCO₃ (20 mL) and was extracted with DCM (2×35 mL). The combined organic layers were dried by Na2SO4, filtered and concentrated under reduced pressure to afford crude was purified by flash chromatography (eluted in 5.5% MeOH:DCM) to afford tert-butyl 4-(4-(4-(benzo[d]thiazol-5-ylamino)quinolin-6-yl)-3-fluorobenzyl)piperazine-1-carboxylate (25 mg, 80.6%), LCMS: 570.3 [M+1]⁺, ¹H NMR (400 MHz, DMSO-d₆) δ ppm 11.05 (br. s., 1H) 9.54 (s, 1H) 8.95 (s, 1H) 8.57 (d, J=7.15 Hz, 2H) 8.40 (d, J=8.58 Hz, 1H) 8.19-8.28 (m, 2H) 8.10 (d, J=8.58 Hz, 2H) 7.56-7.66 (m, 2H) 6.96 (d, J=7.15 Hz, 1H) 3.64 (s, 2H) 3.24 (m, 4H) 2.8 (m, 4H) 1.40 (s, 9H).

### Example 39. Synthesis of 5-(4-(benzo[d]thiazol-5-ylamino)quinolin-6-yl)-2-(pyrrolidin-1-yl)benzonitrile (Compound 39)

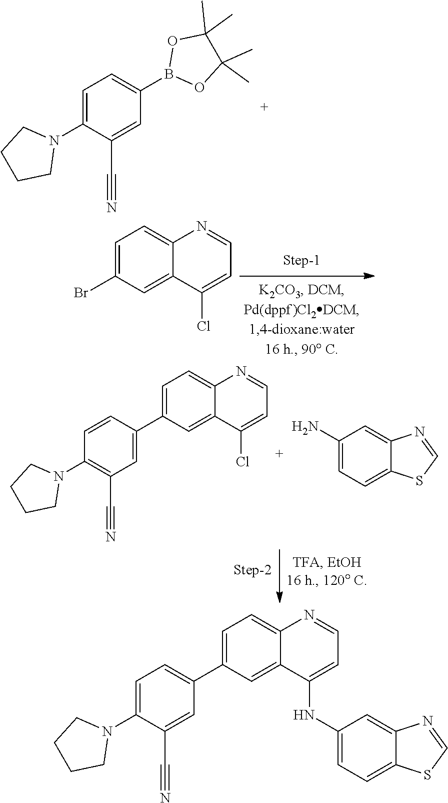

### Step-1: Synthesis of 5-(4-chloroquinolin-6-yl)-2-(pyrrolidin-1-yl)benzonitrile

To a stirred solution of 6-bromo-4-chloroquinoline (100 mg, 0.412 mmol, 1.0 eq) and 2-(pyrrolidin-1-yl)-5-(4,4,5,5-tetramethyl-1,3,2-dioxaborolan-2-yl)benzonitrile (147.6 mg, 0.494 mmol, 1.2 eq) in 1,4-dioxane:water (4 mL:1 mL) was added K₂CO₃ (85.29 mg, 0.618 mmol, 1.5 eq). The reaction mixture was purged with N₂ gas for 10 minutes and was added Pd(dppf)Cl₂·DCM (16.8 mg, 0.0206 mmol, 0.05 eq). Reaction mixture was stirred at 90° C. for 16 h. The progress of reaction was monitored by LCMS and TLC. After completion of the reaction, reaction mixture was diluted with water (50 mL) and was extracted with EtOAc (2×75 mL). The combined organic layers were dried by Na₂SO₄, filtered and concentrated under reduced pressure to afford crude which was purified by flash chromatography (eluted in 4% MeOH:DCM) to afford 5-(4-chloroquinolin-6-yl)-2-(pyrrolidin-1-yl)benzonitrile (50 mg, 27.02%), LCMS: 334.2 [M+1]⁺.

### Step-2: Synthesis of 5-(4-(benzo[d]thiazol-5-ylamino)quinolin-6-yl)-2-(pyrrolidin-1-yl)benzonitrile

To a solution of 5-(4-chloroquinolin-6-yl)-2-(pyrrolidin-1-yl)benzonitrile (50 mg, 0.149 mmol, 1.0 eq) and benzo[d]thiazol-5-amine (22.5 mg, 0.149 mmol, 1.0 eq) in EtOH (2 mL) was added TFA (0.05 mL) and the reaction mixture was heated at 120° C. for 16 h. The progress of reaction was monitored by TLC and LCMS. After completion of reaction, reaction mixture was concentrated under reduced pressure to obtain crude which was basified with saturated solution of NaHCO₃ (10 mL) and was extracted with DCM (2×25 mL). The combined organic layers were dried by Na₂SO₄, filtered and concentrated under reduced pressure to afford crude which was purified by flash chromatography (eluted in 8.5% MeOH:DCM) to afford 5-(4-(benzo[d]thiazol-5-ylamino)quinolin-6-yl)-2-(pyrrolidin-1-yl)benzonitrile (11.05 mg, 17.9%), LCMS: 448.3 [M+1]⁺, ¹H NMR (400 MHz, DMSO-d₆) δ ppm 9.44 (s, 1H) 9.29 (br. s., 1H) 8.66 (s, 1H) 8.44 (d, J=5.25 Hz, 1H) 8.23 (d, J=8.58 Hz, 1H) 8.10 (d, J=2.38 Hz, 1H) 8.03-8.07 (m, 2H) 8.01 (dd, J=9.06, 2.38 Hz, 1H) 7.92 (d, J=8.58 Hz, 1H) 7.57 (d, J=8.58 Hz, 1H) 6.93-6.98 (m, 2H) 3.59-3.62 (m, 4H) 1.99 (br. s., 4H).

### Example 40. Synthesis of N-(6-(4-((4,4-difluoropiperidin-1-yl)methyl)-2-fluorophenyl)quinolin-4-yl)benzo[d]thiazol-5-amine (Compound 40)

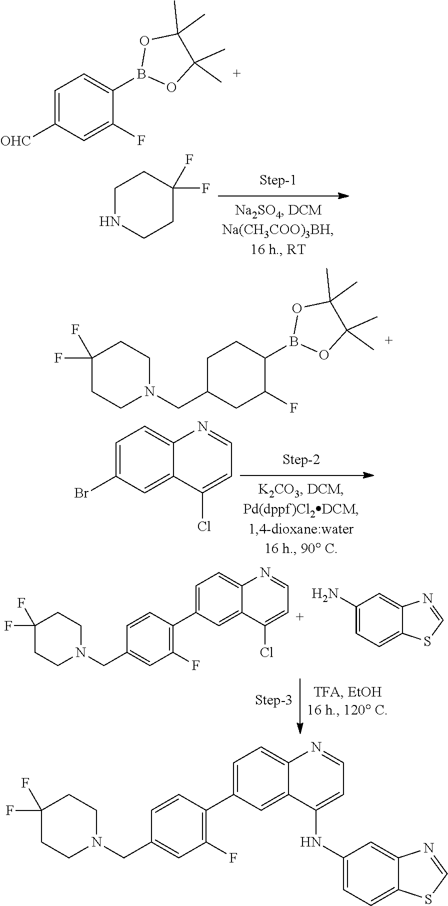

### Step-1: Synthesis of 4,4-difluoro-1-(3-fluoro-4-(4,4,5,5-tetramethyl-1,3,2-dioxaborolan-2-yl)benzyl)piperidine

To a stirred solution of 3-fluoro-4-(4,4,5,5-tetramethyl-1,3,2-dioxaborolan-2-yl)benzaldehyde (125 mg, 0.499 mmol, 1 eq) and 4,4-difluoropiperidine (66.6 mg, 0.549 mmol, 1.1 eq) in DCM (5 mL), was added Na₂SO₄ (141.9 mg, 0.998 mmol, 2.0 eq). Reaction mixture was stirred at RT for 1 h and was added sodium triacetoxyborohydride (158.7 mg, 0.748 mmol, 1.5 eq) portion wise at 0° C. Reaction mixture was stirred at RT for 16 h. The progress of reaction was monitored by LCMS. After completion of the reaction, reaction mixture was diluted with water (50 mL) and extracted with DCM (2×100 mL). The combined organic layers were washed with saturated NaHCO₃ (50 mL) solution, organic layer was dried by Na₂SO₄, filtered and concentrated under reduced pressure to afford 4,4-difluoro-1-(3-fluoro-4-(4,4,5,5-tetramethyl-1,3,2-dioxaborolan-2-yl)benzyl)piperidine (215 mg, 85.65%). LCMS: 274.3 [Boronic-acid Fragment].

### Step-2: Synthesis of 4-chloro-6-(4-((4,4-difluoropiperidin-1-yl)methyl)-2-fluorophenyl)quinoline

To a stirred solution of 6-bromo-4-chloroquinoline (150 mg, 0.612 mmol, 1.0 eq) and 4,4-difluoro-1-(3-fluoro-4-(4,4,5,5-tetramethyl-1,3,2-dioxaborolan-2-yl)benzyl)piperidine (265.3 mg, 0.734 mmol, 1.2 eq) in 1,4-dioxane:water (4 mL:1 mL) was added K₂CO₃ (126.9 mg, 0.918 mmol, 1.5 eq). Reaction mixture was purged with N₂ gas for 10 minutes and was added Pd(dppf)Cl₂·DCM (24.9 mg, 0.030 mmol, 0.05 eq). Reaction mixture was heated at 90° C. for 16 h. The progress of reaction was monitored by LCMS and TLC. After completion of the reaction, reaction mixture was diluted with water (50 mL) and extracted with EtOAc (2×100 mL). The combined organic layers were dried by Na₂SO₄, filtered and concentrated under reduced pressure to afford crude which was purified by flash chromatography (eluted in 2% MeOH:DCM) to afford 4-chloro-6-(4-((4,4-difluoropiperidin-1-yl)methyl)-2-fluorophenyl)quinoline (70 mg, 28.9%), LCMS: 391.2 [M+1]⁺.

### Step-3: Synthesis of N-(6-(4-((4,4-difluoropiperidin-1-yl)methyl)-2-fluorophenyl)quinolin-4-yl)benzo[d]thiazol-5-amine

To a solution of 4-chloro-6-(4-((4,4-difluoropiperidin-1-yl)methyl)-2-fluorophenyl)quinoline (70 mg, 0.179 mmol, 1.0 eq) and benzo[d]thiazol-5-amine (26.9 mg, 0.179 mmol, 1.0 eq) in EtOH (3 mL) was added TFA (0.08 mL). Reaction mixture was heated at 120° C. for 16 h. The progress of reaction was monitored by TLC and LCMS. After completion of the reaction, reaction mixture was concentrated under reduced pressure to obtain crude which was basified with saturated solution of NaHCO₃ (30 mL) and was extracted with DCM (2×45 mL). The combined organic layers were dried by Na2SO4, filtered and concentrated under reduced pressure to afford crude which was purified by flash chromatography (eluted in 12.5% MeOH:DCM) to afford N-(6-(4-((4,4-difluoropiperidin-1-yl)methyl)-2-fluorophenyl)quinolin-4-yl)benzo[d]thiazol-5-amine (33.5 mg, 36.6%), LCMS: 505.3 [M+1]⁺, ¹H NMR (400 MHz, DMSO-d₆) δ ppm 11.09 (br. s., 1H) 9.54 (s, 1H) 8.96 (s, 1H) 8.57 (d, J=6.68 Hz, 1H) 8.40 (d, J=8.58 Hz, 1H) 8.21-8.28 (m, 3H) 8.10 (d, J=9.06 Hz, 2H) 7.62 (dd, J=8.34, 1.67 Hz, 2H) 6.96 (d, J=6.68 Hz, 1H) 3.64 (s, 2H) 3.24 (m, 4H) 2.3 (m, 4H).

### Example 41. Synthesis of N-(6-(2-fluoro-4-(piperazin-1-ylmethyl)phenyl)quinolin-4-yl)benzo[d]thiazol-5-amine (Compound 41)

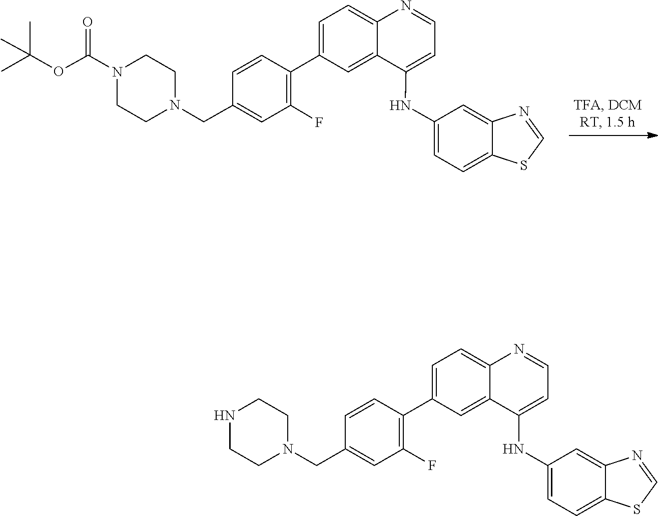 [SMILES: CC(C)(C)OC(=O)N1CCN(Cc2ccc(-c3ccc4nccc(Nc5ccc6scnc6c5)c4c3)c(F)c2)CC1 ; Fc1cc(CN2CCNCC2)ccc1-c1ccc2nccc(Nc3ccc4scnc4c3)c2c1]

### Step-1: Synthesis of N-(6-(2-fluoro-4-(piperazin-1-ylmethyl)phenyl)quinolin-4-yl)benzo[d]thiazol-5-amine

To a solution of tert-butyl 4-(4-(4-(benzo[d]thiazol-5-ylamino)quinolin-6-yl)-3-fluorobenzyl)piperazine-1-carboxylate (20 mg, 0.035 mmol, 1.0 eq) in DCM (1 mL) was added TFA (0.1 mL) at 0° C. for 10 minutes. After addition, reaction mixture was stirred at RT for 1.5 h. The progress of reaction was monitored by TLC and LCMS. After completion of the reaction, reaction mixture was concentrated under reduced pressure to obtain crude which was triturated with diethyl ether (2×5 mL) to afford N-(6-(2-fluoro-4-(piperazin-1-ylmethyl)phenyl)quinolin-4-yl)benzo[d]thiazol-5-amine (14.3 mg, 89.3%), LCMS: 470.3 [M+1]⁺, ¹H NMR (400 MHz, DMSO-d₆) δ ppm 9.54 (s, 1H) 8.95 (s, 1H) 8.58 (d, J=6.68 Hz, 3H) 8.41 (d, J=8.58 Hz, 1H) 8.23 (s, 2H) 8.11 (d, J=8.58 Hz, 1H) 7.74 (t, J=8.11 Hz, 1H) 7.63 (d, J=8.11 Hz, 1H) 7.36-7.46 (m, 2H) 6.95 (d, J=7.15 Hz, 1H) 3.64 (s, 2H) 3.14 (br. s., 4H) 2.64 (br. s., 4H).

### Example 42. Synthesis of N-(6-(2-fluoro-4-((4-(trifluoromethyl)piperidin-1-yl)methyl)phenyl)quinolin-4-yl)benzo[d]thiazol-5-amine (Compound 42)

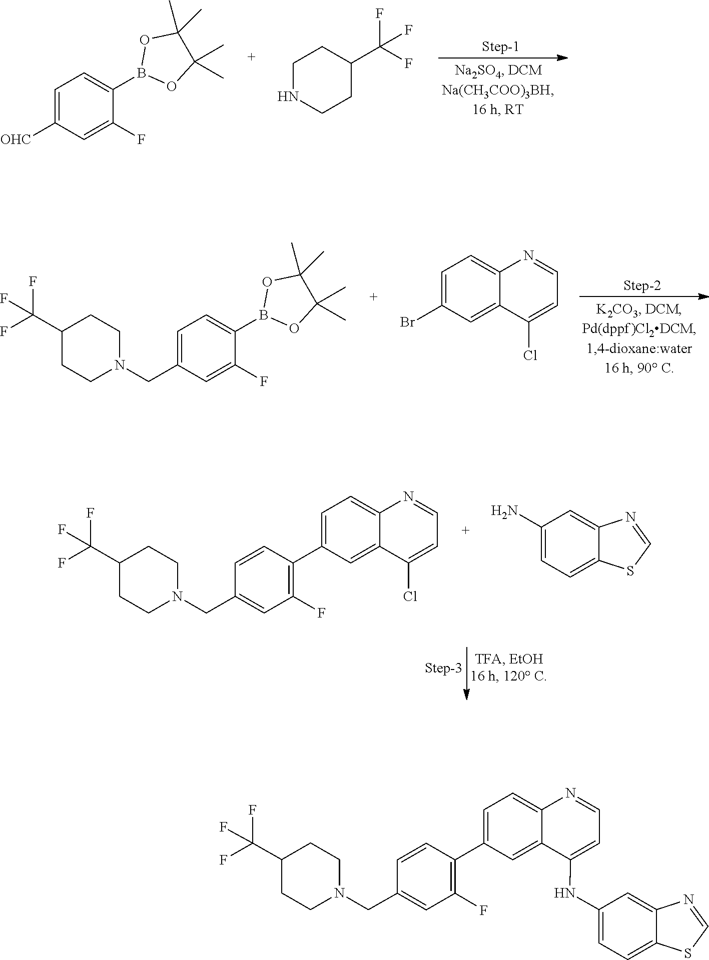

### Step-1: Synthesis of 1-(3-fluoro-4-(4,4,5,5-tetramethyl-1,3,2-dioxaborolan-2-yl)benzyl)-4-(trifluoromethyl)piperidine

To a stirred solution of 3-fluoro-4-(4,4,5,5-tetramethyl-1,3,2-dioxaborolan-2-yl)benzaldehyde (250 mg, 0.999 mmol, 1 eq) and 4-(trifluoromethyl)piperidine (168.4 mg, 1.09 mmol, 1.1 eq) in DCM (5 mL) was added Na₂SO₄ (283.9 mg, 0.1.998 mmol, 2.0 eq). Reaction mixture was stirred at RT for 1 h and was added sodium triacetoxyborohydride (317.5 mg, 1.498 mmol, 1.5 eq) portion wise at 0° C. After addition, Reaction mixture was stirred at RT for 16 h. The progress of reaction was monitored by LCMS. After completion of the reaction, reaction mixture was diluted with water (50 mL) and extracted with DCM (2×100 mL). The combined organic layers were washed with saturated NaHCO₃ (50 mL) solution, organic layer was dried by Na₂SO₄, filtered and concentrated under reduced pressure to afford 1-(3-fluoro-4-(4,4,5,5-tetramethyl-1,3,2-dioxaborolan-2-yl)benzyl)-4-(trifluoromethyl)piperidine (316 mg, 81.86%), LCMS: 306.3 [Boronic-acid Fragment].

### Step-2: Synthesis of 4-chloro-6-(2-fluoro-4-((4-(trifluoromethyl)piperidin-1-yl)methyl)phenyl)quinoline

To a stirred solution of 6-bromo-4-chloroquinoline (150 mg, 0.612 mmol, 1.0 eq) and 1-(3-fluoro-4-(4,4,5,5-tetramethyl-1,3,2-dioxaborolan-2-yl)benzyl)-4-(trifluoromethyl)piperidine (286.9 mg, 0.740 mmol, 1.2 eq) in 1,4-dioxane:water (4 mL:1 mL) was added K₂CO₃ (127.9 mg, 0.925 mmol, 1.5 eq). Reaction mixture was purged with N₂ gas for 10 minutes and was added Pd(dppf)Cl₂·DCM (25.19 mg, 0.030 mmol, 0.05 eq). Reaction mixture was heated at 90° C. for 16 h. The progress of reaction was monitored by ¹H NMR and TLC. After completion of the reaction, reaction mixture was diluted with water (50 mL) and extracted with EtOAc (2×100 mL). The combined organic layers were dried by Na₂SO₄, filtered and concentrated under reduced pressure to afford crude product which was purified by flash chromatography (eluted in 2% MeOH:DCM) to afford 4-chloro-6-(2-fluoro-4-((4-(trifluoromethyl)piperidin-1-yl)methyl)phenyl)quinoline (61 mg, 23.19%), ¹H NMR (400 MHz, CHLOROFORM-d) δ ppm 8.80 (d, J=4.77 Hz, 1H) 8.39 (s, 1H) 8.19 (d, J=8.58 Hz, 1H) 7.96-8.01 (m, 1H) 7.53 (d, J=4.77 Hz, 2H) 7.22 (d, J=3.81 Hz, 2H) 3.57 (s, 2H) 3.20 (m, 4H) 2.15 (br. s., 1H) 1.68 (m, 4H).

### Step-3: Synthesis of N-(6-(2-fluoro-4-((4-(trifluoromethyl)piperidin-1-yl)methyl)phenyl)quinolin-4-yl)benzo[d]thiazol-5-amine

To a solution of 4-chloro-6-(2-fluoro-4-((4-(trifluoromethyl)piperidin-1-yl)methyl)phenyl)quinoline (30 mg, 0.070 mmol, 1.0 eq) and benzo[d]thiazol-5-amine (10.65 mg, 0.070 mmol, 1.0 eq) in EtOH (2 mL) was added TFA (0.05 mL). After addition, reaction mixture was heated at 120° C. for 16 h. The progress of the reaction was monitored by TLC and LCMS. After completion of reaction, reaction mixture was concentrated under reduced pressure to obtain crude which was basified with saturated solution of NaHCO₃ (30 mL) and was extracted with DCM (2×45 mL). The combined organic layers were dried by Na₂SO₄, filtered and concentrated under reduced pressure to afford crude product which was purified by flash chromatography (eluted in 12.5% MeOH:DCM) to afford N-(6-(2-fluoro-4-((4-(trifluoromethyl)piperidin-1-yl)methyl)phenyl)quinolin-4-yl)benzo[d]thiazol-5-amine (32.5 mg, 84.2%), LCMS: 537.3 [M+1]⁺, ¹H NMR (400 MHz, DMSO-d₆) δ ppm 11.07 (br. s., 1H) 9.54 (s, 1H) 8.97 (s, 1H) 8.58 (d, J=6.68 Hz, 1H) 8.41 (s, 1H) 8.20-8.27 (m, 3H) 8.12 (d, J=9.06 Hz, 2H) 7.83 (br. s., 1H) 7.62 (dd, J=8.58, 1.91 Hz, 1H) 6.96 (d, J=6.68 Hz, 1H) 3.64 (s, 2H) 3.24 (m, 4H) 2.16 (br. s., 1H) 1.9 (m, 4H).

### Example 43. Synthesis of N-(7-(2-fluoro-4-(piperidin-1-ylmethyl)phenyl)quinolin-4-yl)benzo[d]thiazol-5-amine (Compound 43)

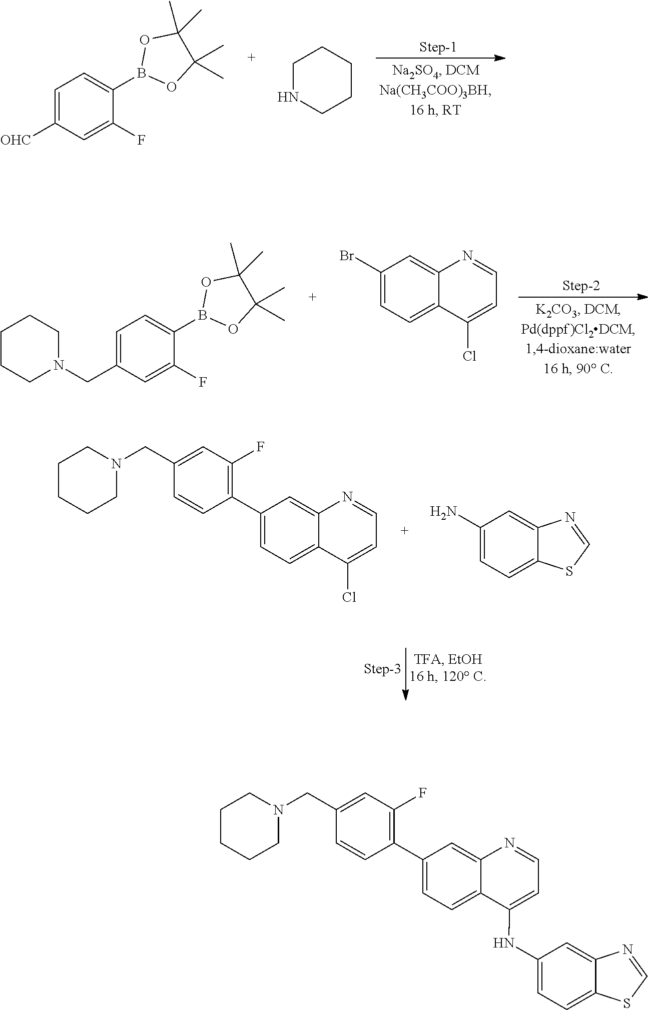

### Step-1: Synthesis of 1-(3-fluoro-4-(4,4,5,5-tetramethyl-1,3,2-dioxaborolan-2-yl)benzyl)piperidine

To a stirred solution of 3-fluoro-4-(4,4,5,5-tetramethyl-1,3,2-dioxaborolan-2-yl)benzaldehyde (400 mg, 1.60 mmol, 1 eq) and piperidine (136.2 mg, 1.60 mmol, 1.0 eq) in DCM (15 mL) was added Na₂SO₄ (455 mg, 3.20 mmol, 2.0 eq). Reaction mixture was stirred at RT for 1 h and was added sodium triacetoxyborohydride (509 mg, 2.4 mmol, 1.5 eq) portion wise at 0° C. After addition, reaction mixture was stirred at RT for 16 h. The progress of reaction was monitored by LCMS. After completion of reaction, reaction mixture was diluted with water (75 mL) and was extracted with DCM (2×100 mL). The combined organic layers were washed with saturated NaHCO₃ solution (50 mL), organic layer was dried over anhydrous Na₂SO₄, filtered and concentrated under reduced pressure to afford 1-(3-fluoro-4-(4,4,5,5-tetramethyl-1,3,2-dioxaborolan-2-yl)benzyl)piperidine (400 mg, 78.43%), LCMS: 238.3 [Boronic-acid Fragment].

### Step-2: Synthesis of 4-chloro-7-(2-fluoro-4-(piperidin-1-ylmethyl)phenyl)quinoline

To a stirred solution of 7-bromo-4-chloroquinoline (250 mg, 1.04 mmol, 1.0 eq) and 1-(3-fluoro-4-(4,4,5,5-tetramethyl-1,3,2-dioxaborolan-2-yl)benzyl)piperidine (398.37 mg, 1.23 mmol, 1.2 eq) in 1,4-dioxane:water (6 mL:1.5 mL) was added K₂CO₃ (215.3 mg, 1.56 mmol, 1.5 eq). Reaction mixture was purged with N₂ gas for 10 minutes and was added Pd(dppf)Cl₂·DCM (42.4 mg, 0.052 mmol, 0.05 eq). Reaction mixture was heated at 90° C. for 16 h. The progress of reaction was monitored by LCMS and TLC. After completion of reaction, reaction mixture was diluted with water (50 mL) and was extracted with EtOAc (2×75 mL), combined organic layers were dried over Na2SO4, filtered and concentrated under reduced pressure to afford crude which was purified by flash chromatography (eluted in 2% MeOH:DCM) to afford 4-chloro-7-(2-fluoro-4-(piperidin-1-ylmethyl)phenyl)quinoline (100 mg, 54.3%), LCMS: 355.3 [M+1]⁺.

### Step-3: Synthesis of N-(7-(2-fluoro-4-(piperidin-1-ylmethyl)phenyl)quinolin-4-yl)benzo[d]thiazol-5-amine

To a solution of 4-chloro-7-(2-fluoro-4-(piperidin-1-ylmethyl)phenyl)quinoline (50 mg, 0.141 mmol, 1.0 eq) and benzo[d]thiazol-5-amine (21.16 mg, 0.141 mmol, 1.0 eq) in EtOH (2 mL) was added TFA (0.05 mL). Reaction mixture was heated at 120° C. for 16 h. The progress of reaction was monitored by TLC and LCMS. After completion of reaction, reaction mixture was concentrated under reduced pressure to obtain crude which was basified with saturated NaHCO₃ solution (25 mL) and was extracted with DCM (2×50 mL). The combined organic layers were dried over Na2SO4, filtered and concentrated under reduced pressure to afford which was purified by flash chromatography (eluted in 6% MeOH:DCM) to afford N-(7-(2-fluoro-4-(piperidin-1-ylmethyl)phenyl)quinolin-4-yl)benzo[d]thiazol-5-amine (17.8 mg, 27.2%), LCMS: 469.3 [M+1]⁺, ¹H NMR (400 MHz, DMSO-d₆) δ ppm 9.43 (s, 1H) 9.29 (br. s., 1H) 8.61 (s, 1H) 8.51 (d, J=5.25 Hz, 1H) 8.20 (d, J=8.58 Hz, 1H) 8.03 (d, J=1.91 Hz, 1H) 7.97 (d, J=8.58 Hz, 1H) 7.89 (d, J=9.06 Hz, 1H) 7.68 (t, J=8.11 Hz, 1H) 7.55 (dd, J=8.58, 1.91 Hz, 1H) 7.25-7.33 (m, 2H) 7.05 (d, J=5.25 Hz, 1H) 3.61 (s, 2H) 3.54 (br. s., 4H) 1.50-1.57 (m, 4H) 1.41 (br. s., 2H).

### Example 44. Synthesis of N-(6-(4-(2-(piperidin-1-yl)ethoxy)phenyl)quinolin-4-yl)benzo[d]thiazol-5-amine (Compound 44)

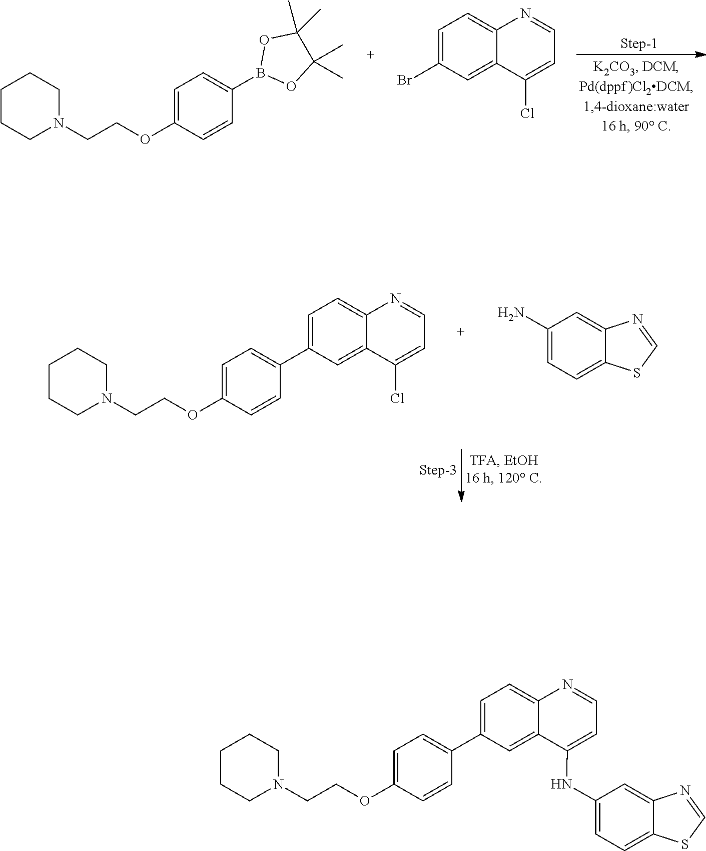 [SMILES: CC1(C)OB(c2ccc(OCCN3CCCCC3)cc2)OC1(C)C ; Clc1ccnc2ccc(Br)cc12 ; Clc1ccnc2ccc(-c3ccc(OCCN4CCCCC4)cc3)cc12 ; Nc1ccc2scnc2c1 ; c1cc(Nc2ccc3scnc3c2)c2cc(-c3ccc(OCCN4CCCCC4)cc3)ccc2n1]

### Step-1: Synthesis of 4-chloro-6-(4-(2-(piperidin-1-yl)ethoxy)phenyl)quinoline

To a stirred solution of 6-bromo-4-chloroquinoline (100 mg, 0.412 mmol, 1.0 eq) and 1-(2-(4-(4,4,5,5-tetramethyl-1,3,2-dioxaborolan-2-yl)phenoxy)ethyl)piperidine (150.2 mg, 0.453 mmol, 1.1 eq) in 1,4-dioxane:water (4 mL:1 mL) was added K₂CO₃ (85.5 mg, 0.619 mmol, 1.5 eq). Reaction mixture was purged with N₂ gas for 10 minutes and was added Pd(dppf)Cl₂·DCM (16.9 mg, 0.021 mmol, 0.05 eq). Reaction mixture was heated at 90° C. for 16 h. The progress of reaction was monitored by LCMS and TLC. After completion reaction, reaction mixture was diluted with water (50 mL) and was extracted with EtOAc (2×75 mL). The combined organic layers were dried by Na₂SO₄, filtered and concentrated under reduced pressure to afford crude product which was purified by flash chromatography (eluted in 4% MeOH:DCM) to afford 4-chloro-6-(4-(2-(piperidin-1-yl)ethoxy)phenyl)quinoline (120 mg, 78.99%). LCMS: 367.4 [M+1]⁺.

### Step-2: Synthesis of N-(6-(4-(2-(piperidin-1-yl)ethoxy)phenyl)quinolin-4-yl)benzo[d]thiazol-5-amine

To a solution of 4-chloro-6-(4-(2-(piperidin-1-yl)ethoxy)phenyl)quinoline (75 mg, 0.204 mmol, 1.0 eq) and benzo[d]thiazol-5-amine (33.77 mg, 0.224 mmol, 1.0 eq) in EtOH (3 mL) was added TFA (0.05 mL). The reaction mixture was heated at 120° C. for 16 h. The progress of reaction was monitored by TLC and LCMS. After completion of reaction, reaction mixture was concentrated under reduced pressure to obtain crude which was basified with saturated solution of NaHCO₃ (20 mL) and was extracted with DCM (2×50 mL). The combined organic layers were dried by Na₂SO₄, filtered and concentrated under reduced pressure to afford crude product which was purified by flash chromatography (eluted in 5.5% MeOH:DCM) to afford N-(6-(4-(2-(piperidin-1-yl)ethoxy)phenyl)quinolin-4-yl)benzo[d]thiazol-5-amine (75.14 mg, 76.53%), LCMS: 481.3 [M+1]⁺, ¹H NMR (400 MHz, DMSO-d₆) δ ppm 9.53 (s, 1H) 9.05 (s, 1H) 8.50 (d, J=6.68 Hz, 1H) 8.31-8.40 (m, 2H) 8.23 (s, 1H) 8.09 (d, J=9.06 Hz, 2H) 7.98 (d, J=8.58 Hz, 2H) 7.64 (d, J=8.58 Hz, 1H) 7.21 (d, J=8.58 Hz, 2H) 6.92 (d, J=6.68 Hz, 1H) 4.48 (br. s., 2H) 3.50 (br. s., 2H) 2.67 (br. s., 4H) 1.80 (br. s., 6H).

### Example 45. Synthesis of 1-(4-(4-(benzo[d]thiazol-5-ylamino)quinolin-6-yl)-3-fluorobenzyl)pyridin-2(1H)-one (Compound 47)

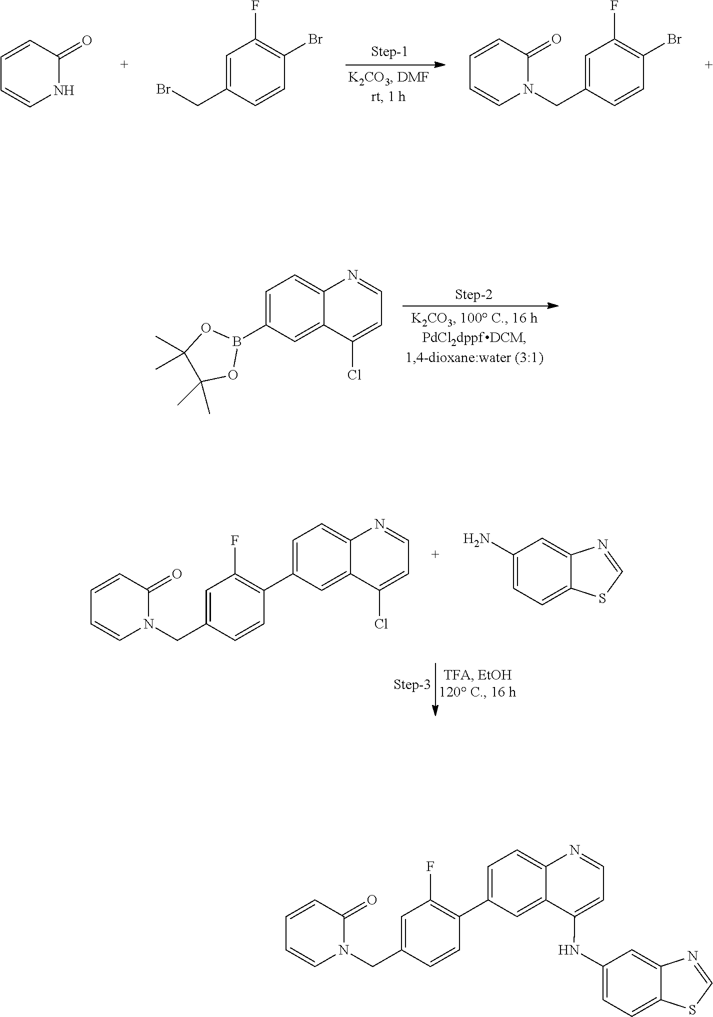

### Step-1: Synthesis of 1-(4-bromo-3-fluorobenzyl)pyridin-2(1H)-one

To a solution of pyridin-2(1H)-one (500 mg, 1.86 mmol, 1 eq) in DMF (5 mL) was added K₂CO₃ (513 mg, 3.73 mmol, 2 eq) and 1-bromo-4-(bromomethyl)-2-fluorobenzene (195 mg, 2.05 mmol, 1.1 eq). The reaction was heated at room temperature for 1 h. The progress of reaction was monitored by TLC. After completion of the reaction, reaction mixture was diluted with water (20 mL) and extracted with ethyl acetate (2×40 mL),washed with brine solution, dried over anhydrous sodium sulphate, filtered and concentrated under vacuum to afford crude which was purified by flash chromatography to afford 1-(4-bromo-3-fluorobenzyl)pyridin-2(1H)-one (520 mg, 98.4%), LCMS: 284.2[M+2]⁺.

### Step-2: Synthesis of 1-(4-(4-chloroquinolin-6-yl)-3-fluorobenzyl)pyridin-2(1H)-one

To a solution of 1-(4-bromo-3-fluorobenzyl)pyridin-2(1H)-one (200 mg, 0.708 mmol, 1 eq) in 1,4-dioxane:water (6 mL:2 mL) was added K₂CO₃ (195 mg, 1.41 mmol, 2 eq) and 4-chloro-6-(4,4,5,5-tetramethyl-1,3,2-dioxaborolan-2-yl)quinoline (205 mg, 0.708 mmol, 1 eq). Reaction mixture was purged with nitrogen gas for 10 min, was added PdCl₂dppf·DCM (28 mg, 0.035 mmol, 0.05 eq). The reaction was heated at 100° C. for 16 h. The progress of reaction was monitored by TLC. After completion of the reaction, reaction mixture was diluted with water (30 mL) and extracted with ethyl acetate (2×50 mL), dried over anhydrous sodium sulphate, filtered and concentrated under vacuum to afford crude which was purified by flash chromatography to afford 1-(4-(4-chloroquinolin-6-yl)-3-fluorobenzyl)pyridin-2(1H)-one (100 mg, 38.7%), LCMS: 365.3[M+1]⁺.

### Step-3: Synthesis of 1-(4-(4-(benzo[d]thiazol-5-ylamino)quinolin-6-yl)-3-fluorobenzyl)pyridin-2(1H)-one

To a solution of 1-(4-(4-chloroquinolin-6-yl)-3-fluorobenzyl)pyridin-2(1H)-one (100 mg, 0.274 mmol, 1.0 eq) in ethanol (5 mL) was added benzo[d]thiazol-5-amine (61 mg, 0.411 mmol, 1.5 eq) and TFA (0.1 mL). The reaction mixture was heated at 120° C. for 16 h. After completion of the reaction, reaction mixture was concentrated under vacuum to obtain crude which was neutralized by saturated sodium bicarbonate solution (20 mL), was extracted with dichloromethane (2×40 mL), dried over anhydrous sodium sulphate, filtered and concentrated under vacuum to afford crude which was purified by flash chromatography to afford 1-(4-(4-(benzo[d]thiazol-5-ylamino)quinolin-6-yl)-3-fluorobenzyl)pyridin-2(1H)-one (18 mg, 13.7%), LCMS: 479.2 [M+1]⁺, ¹H NMR (400 MHz, DMSO-d₆) δ ppm 5.18 (s, 2H) 6.29 (t, J=6.68 Hz, 1H) 6.45 (d, J=9.06 Hz, 1H) 7.05 (d, J=4.77 Hz, 1H) 7.29-7.33 (m, 2H) 7.46 (t, J=7.87 Hz, 1H) 7.54 (d, J=8.58 Hz, 1H) 7.68-7.73 (m, 1H) 7.88 (br. s., 2H) 7.97 (s, 1H) 8.02 (s, 1H) 8.20 (d, J=9.06 Hz, 1H) 8.51 (d, J=5.25 Hz, 1H) 8.58 (s, 1H) 9.23 (s, 1H) 9.43 (s, 1H).

### Example 46. Synthesis of N-(7-(4-(2-(piperidin-1-yl)ethoxy)phenyl)quinolin-4-yl)benzo[d]thiazol-5-amine (Compound 50)

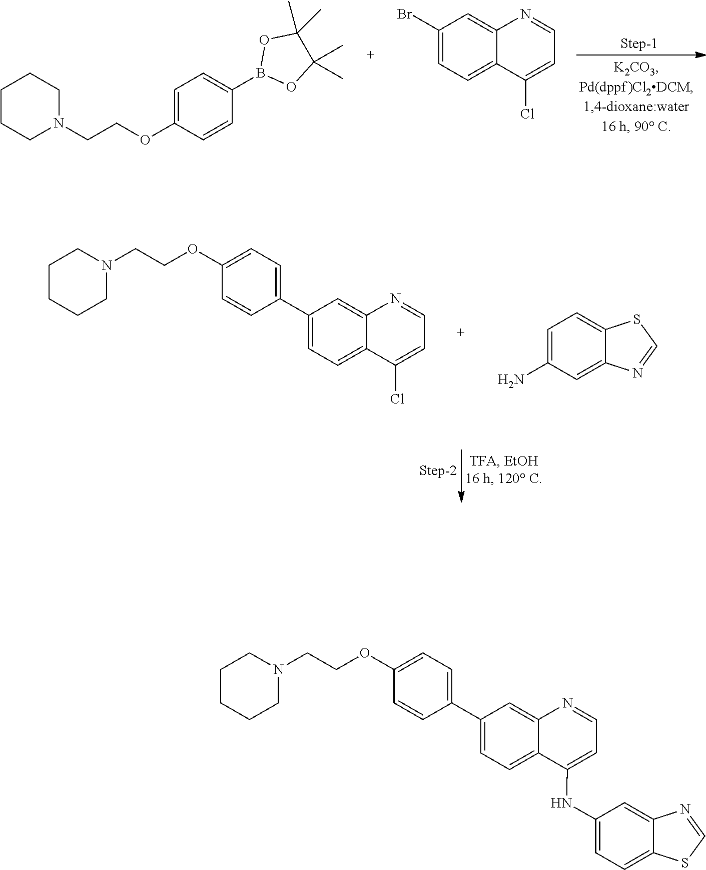 [SMILES: CC1(C)OB(c2ccc(OCCN3CCCCC3)cc2)OC1(C)C ; Clc1ccnc2cc(Br)ccc12 ; Clc1ccnc2cc(-c3ccc(OCCN4CCCCC4)cc3)ccc12 ; Nc1ccc2scnc2c1 ; c1cc(Nc2ccc3scnc3c2)c2ccc(-c3ccc(OCCN4CCCCC4)cc3)cc2n1]

### Step-1: Synthesis of 4-chloro-7-(4-(2-(piperidin-1-yl)ethoxy)phenyl)quinoline

To a stirred solution of 7-bromo-4-chloroquinoline (150 mg, 0.618 mmol, 1.0 eq) and 1-(2-(4-(4,4,5,5-tetramethyl-1,3,2-dioxaborolan-2-yl)phenoxy)ethyl)piperidine (246.1 mg, 0.743 mmol, 1.1 eq) in 1,4-dioxane:water (4 mL:1 mL) was added K₂CO₃ (127.9 mg, 0.927 mmol, 1.5 eq). Reaction mixture was purged with N₂ gas for 10 minutes and was added Pd(dppf)Cl₂·DCM (25.3 mg, 0.030 mmol, 0.05 eq). Reaction mixture was heated at 90° C. for 16 h. The progress of reaction was monitored by LCMS and TLC. After completion of the reaction, reaction mixture was diluted with water (50 mL) and was extracted with EtOAc (2×75 mL). The combined organic layers were dried by Na₂SO₄, filtered and concentrated under reduced pressure to afford crude product which was purified by flash chromatography (eluted in 4% MeOH:DCM) to afford 4-chloro-7-(4-(2-(piperidin-1-yl)ethoxy)phenyl)quinoline (90 mg, 39.6%). LCMS: 367.3 [M+1]⁺.

### Step-2: Synthesis of N-(7-(4-(2-(piperidin-1-yl)ethoxy)phenyl)quinolin-4-yl)benzo[d]thiazol-5-amine

To a solution of 4-chloro-7-(4-(2-(piperidin-1-yl)ethoxy)phenyl)quinoline (90 mg, 0.245 mmol, 1.0 eq) and benzo[d]thiazol-5-amine (36.99 mg, 0.245 mmol, 1.0 eq) in EtOH (4 mL) was added TFA (0.1 mL). The reaction mixture was heated at 120° C. for 16 h. The progress of reaction was monitored by TLC and LCMS. After completion of the reaction, reaction mixture was concentrated under reduced pressure to obtain crude which was neutralized with saturated solution of NaHCO₃ (30 mL) and was extracted with DCM (2×50 mL). The combined organic layers were dried by Na₂SO₄, filtered and concentrated under reduced pressure to afford crude product which was purified by flash chromatography (eluted in 5.5% MeOH:DCM) to afford N-(7-(4-(2-(piperidin-1-yl)ethoxy)phenyl)quinolin-4-yl)benzo[d]thiazol-5-amine (78.5 mg, 66.1%), LCMS: 481.4 [M+1]⁺, ¹H NMR (400 MHz, DMSO-d₆) δ ppm 9.53 (s, 1H) 9.05 (s, 1H) 8.50 (d, J=6.68 Hz, 1H) 8.31-8.40 (m, 2H) 8.23 (s, 1H) 8.09 (d, J=9.06 Hz, 2H) 7.98 (d, J=8.58 Hz, 2H) 7.64 (d, J=8.58 Hz, 1H) 7.21 (d, J=8.58 Hz, 2H) 6.92 (d, J=6.68 Hz, 1H) 4.48 (br. s., 2H) 3.50 (br. s., 2H) 2.67 (br. s., 4H) 1.80 (br. s., 6H).

### Example 47. Synthesis of N-(6-(4-(7-oxa-2-azaspiro[3.5]nonan-2-ylmethyl)-2-fluorophenyl)quinolin-4-yl)benzo[d]thiazol-5-amine (Compound 51)

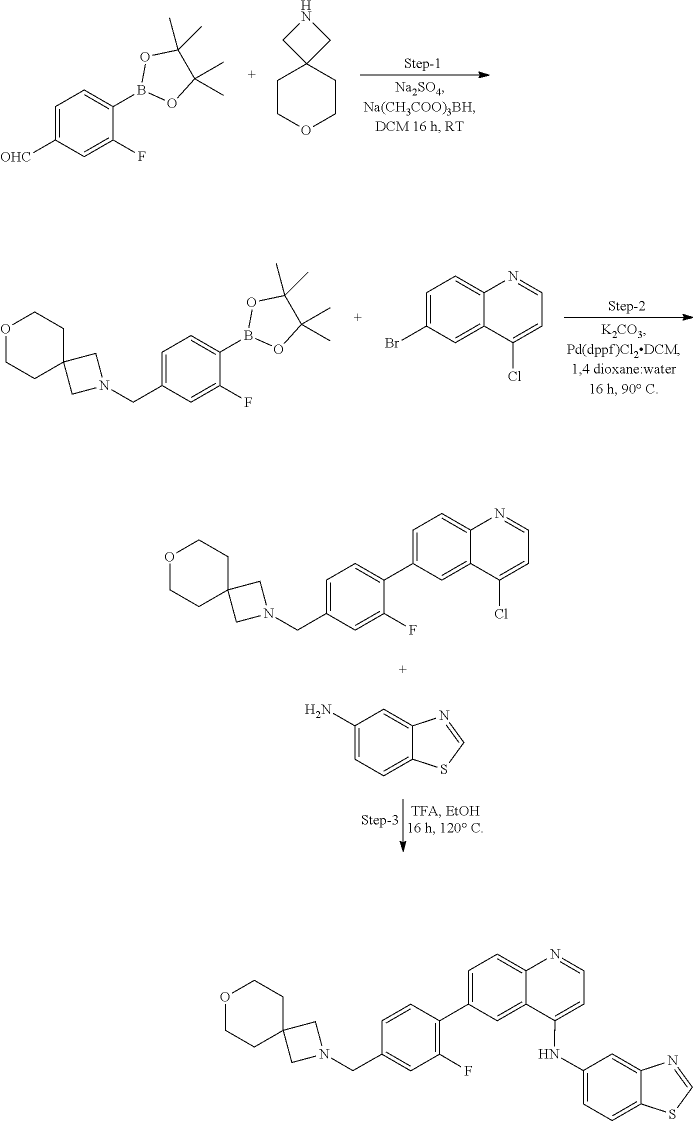

### Step-1: Synthesis of 2-(3-fluoro-4-(4,4,5,5-tetramethyl-1,3,2-dioxaborolan-2-yl)benzyl)-7-oxa-2-azaspiro[3.5]nonane

To a stirred solution of 3-fluoro-4-(4,4,5,5-tetramethyl-1,3,2-dioxaborolan-2-yl)benzaldehyde (300 mg, 1.20 mmol, 1 eq) and 7-oxa-2-azaspiro[3.5]nonane (152.6 mg, 1.20 mmol, 1.0 eq) in DCM (10 mL) was added Na₂SO₄ (341.2 mg, 2.40 mmol, 2.0 eq). Reaction mixture was stirred at RT for 1 h and was added sodium triacetoxyborohydride (381.5 mg, 1.8 mmol, 1.5 eq) portion wise at 0° C. After addition, reaction mixture was stirred at RT for 16 h. The progress of reaction was monitored by LCMS. After completion of the reaction, reaction mixture was diluted with water (75 mL) and was extracted with DCM (2×100 mL). The combined organic layers were washed with saturated NaHCO₃ solution (50 mL), organic layer was dried over anhydrous Na₂SO₄, filtered and concentrated under reduced pressure to afford 2-(3-fluoro-4-(4,4,5,5-tetramethyl-1,3,2-dioxaborolan-2-yl)benzyl)-7-oxa-2-azaspiro[3.5]nonane (395 mg, 91.22%), LCMS: 280.3 [Boronic-acid Fragment].

### Step-2: Synthesis of 2-(4-(4-chloroquinolin-6-yl)-3-fluorobenzyl)-7-oxa-2-azaspiro[3.5]nonane

To a stirred solution of 6-bromo-4-chloroquinoline (100 mg, 0.41 mmol, 1.0 eq) and 2-(3-fluoro-4-(4,4,5,5-tetramethyl-1,3,2-dioxaborolan-2-yl)benzyl)-7-oxa-2-azaspiro[3.5]nonane (148.9 mg, 0.412 mmol, 1.0 eq) in 1,4-dioxane:water (4 mL:1 mL) was added K₂CO₃ (85.3 mg, 0.618 mmol, 1.5 eq). Reaction mixture was purged with N₂ gas for 10 minutes and was added Pd(dppf)Cl₂·DCM (16.9 mg, 0.020 mmol, 0.05 eq). Reaction mixture was heated at 90° C. for 16 h. The progress of reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford 2-(4-(4-chloroquinolin-6-yl)-3-fluorobenzyl)-7-oxa-2-azaspiro[3.5]nonane (60 mg, 36.5%), LCMS: 397.3 [M+1]⁺.

### Step-3: Synthesis of N-(6-(4-(7-oxa-2-azaspiro[3.5]nonan-2-ylmethyl)-2-fluorophenyl)quinolin-4-yl)benzo[d]thiazol-5-amine

To a solution of 2-(4-(4-chloroquinolin-6-yl)-3-fluorobenzyl)-7-oxa-2-azaspiro[3.5]nonane (35 mg, 0.088 mmol, 1.0 eq) and benzo[d]thiazol-5-amine (15.9 mg, 0.106 mmol, 1.2 eq) in EtOH (3 mL) was added TFA (0.04 mL). Reaction mixture was heated at 120° C. for 16 h. The progress of reaction was monitored by TLC and LCMS. After completion of the reaction, reaction mixture was concentrated under reduced pressure to obtain crude which was basified with saturated NaHCO₃ solution (15 mL) and was extracted with DCM (2×30 mL). The combined organic layers were dried over Na2SO4, filtered and concentrated under reduced pressure to afford crude which was purified by flash chromatography (eluted in 5.6% MeOH:DCM) to afford N-(6-(4-(7-oxa-2-azaspiro[3.5]nonan-2-ylmethyl)-2-fluorophenyl)quinolin-4-yl)benzo[d]thiazol-5-amine (11.3 mg, 26.6%), LCMS: 511.3 [M+1]⁺, ¹H NMR (400 MHz, DMSO-d₆) δ ppm 9.43 (s, 1H) 9.28 (s, 1H) 8.60 (s, 1H) 8.51 (d, J=4.77 Hz, 1H) 8.20 (d, J=8.58 Hz, 1H) 8.03 (s, 1H) 7.94-7.98 (m, 1H) 7.89 (s, 1H) 7.63-7.68 (m, 1H) 7.54 (s, 1H) 7.24-7.30 (m, 2H) 7.05 (d, J=5.25 Hz, 1H) 3.69 (s., 2H) 3.49 (t, J=5.01 Hz, 4H) 3.04 (br. s., 4H) 1.68 (t, J=5.01 Hz, 4H).

### Example 48. Synthesis of N-(7-(4-(7-oxa-2-azaspiro[3.5]nonan-2-ylmethyl)-2-fluorophenyl)quinolin-4-yl)benzo[d]thiazol-5-amine (Compound 52)

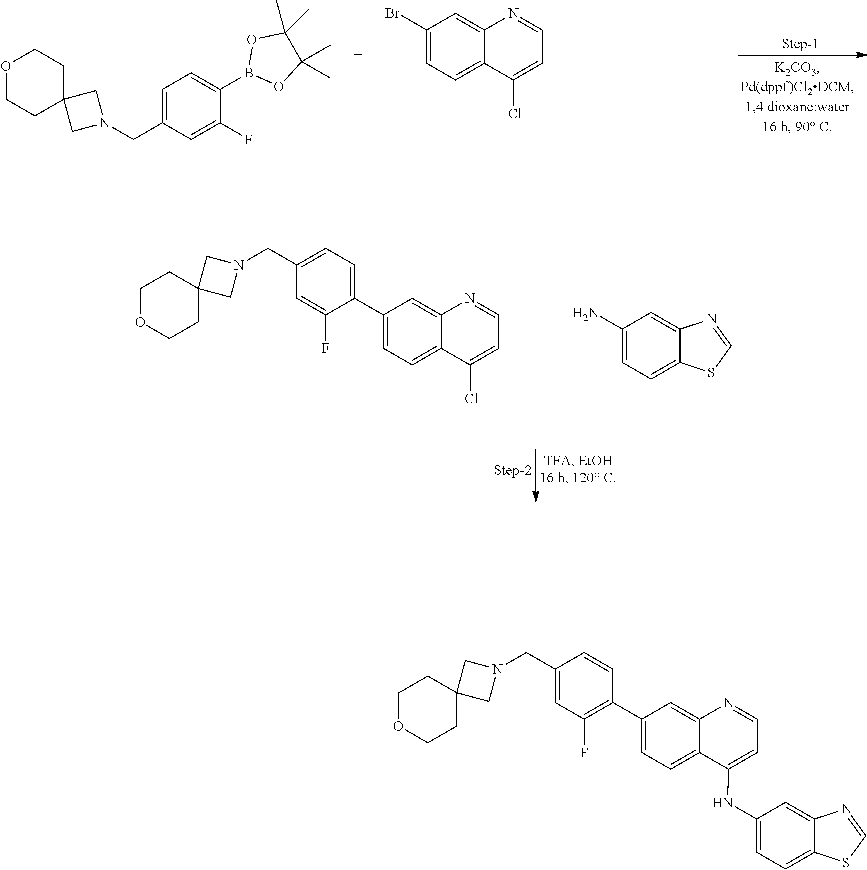 [SMILES: CC1(C)OB(c2ccc(CN3CC4(CCOCC4)C3)cc2F)OC1(C)C ; Clc1ccnc2cc(Br)ccc12 ; Fc1cc(CN2CC3(CCOCC3)C2)ccc1-c1ccc2c(Cl)ccnc2c1 ; Nc1ccc2scnc2c1 ; Fc1cc(CN2CC3(CCOCC3)C2)ccc1-c1ccc2c(Nc3ccc4scnc4c3)ccnc2c1]

### Step-1: Synthesis of 2-(4-(4-chloroquinolin-7-yl)-3-fluorobenzyl)-7-oxa-2-azaspiro[3.5]nonane

To a stirred solution of 7-bromo-4-chloroquinoline (100 mg, 0.41 mmol, 1.0 eq) and 2-(3-fluoro-4-(4,4,5,5-tetramethyl-1,3,2-dioxaborolan-2-yl)benzyl)-7-oxa-2-azaspiro[3.5]nonane (178.8 mg, 0.494 mmol, 1.2 eq) in 1,4-dioxane:water (5 mL:1 mL) was added K₂CO₃ (85.3 mg, 0.618 mmol, 1.5 eq). Reaction mixture was purged with N₂ gas for 10 minutes and was added Pd(dppf)Cl₂·DCM (16.9 mg, 0.020 mmol, 0.05 eq). Reaction mixture was heated at 90° C. for 16 h. The progress of reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford 2-(4-(4-chloroquinolin-7-yl)-3-fluorobenzyl)-7-oxa-2-azaspiro[3.5]nonane (60 mg, 36.5%), LCMS: 397.3 [M+1]⁺.

### Step-2: Synthesis of N-(7-(4-(7-oxa-2-azaspiro[3.5]nonan-2-ylmethyl)-2-fluorophenyl)quinolin-4-yl)benzo[d]thiazol-5-amine

To a solution of 2-(4-(4-chloroquinolin-7-yl)-3-fluorobenzyl)-7-oxa-2-azaspiro[3.5]nonane (25 mg, 0.063 mmol, 1.0 eq) and benzo[d]thiazol-5-amine (11.4 mg, 0.076 mmol, 1.2 eq) in EtOH (2 mL) was added TFA (0.025 mL). Reaction mixture was heated at 120° C. for 16 h. The progress of reaction was monitored by TLC and LCMS. After completion of the reaction, reaction mixture was concentrated under reduced pressure to obtain crude which was neutralized with saturated NaHCO₃ solution (10 mL) and was extracted with DCM (2×25 mL). The combined organic layers were dried over Na₂SO₄, filtered and concentrated under reduced pressure to afford which was purified by flash chromatography (eluted in 5.6% MeOH:DCM) to afford N-(7-(4-(7-oxa-2-azaspiro[3.5]nonan-2-ylmethyl)-2-fluorophenyl)quinolin-4-yl)benzo[d]thiazol-5-amine (5.4 mg, 15.6%), LCMS: 511.3 [M+1]⁺, ¹H NMR (400 MHz, DMSO-d₆) δ ppm 9.43 (s, 1H) 9.26 (s, 1H) 8.49-8.54 (m, 2H) 8.20 (d, J=8.58 Hz, 1H) 8.05 (s, 2H) 7.75 (d, J=8.58 Hz, 1H) 7.65-7.71 (m, 1H) 7.54-7.59 (m, 1H) 7.25-7.31 (m, 2H) 7.04 (d, J=5.25 Hz, 1H) 3.71 (s., 2H) 3.46-3.52 (m, 4H) 3.09 (br. s., 4H) 1.69 (t, J=5.25 Hz, 4H).

### Example 49. Synthesis of (4-(4-(benzo[d]thiazol-5-ylamino)quinolin-6-yl)-3-fluorophenyl)(piperidin-1-yl)methanone (Compound 53)

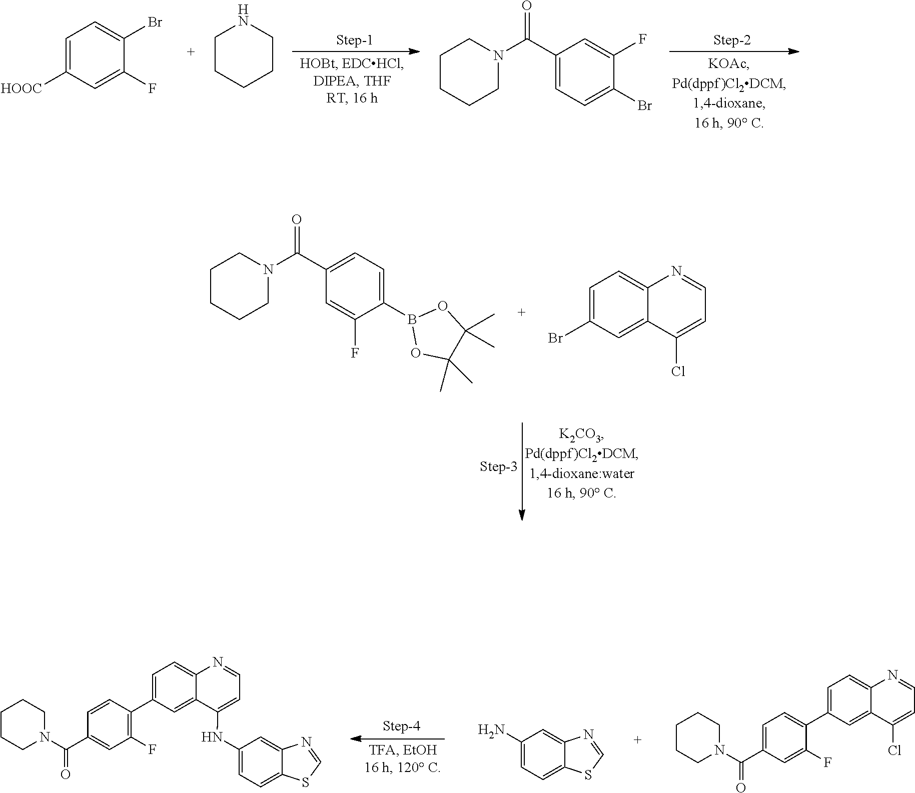

### Step-1: Synthesis of (4-bromo-3-fluorophenyl)(piperidin-1-yl)methanone

To a stirred solution of 4-bromo-3-fluorobenzoic acid (100 mg, 0.458 mmol, 1.0 eq) in THF (3 mL), was added HOBt (80.5 mg, 0.595 mmol, 1.3 eq) and EDC·HCl (114.13 mg, 0.595 mmol, 1.0 eq). Reaction mixture was stirred at RT for 1 h. Then was added DIPEA (0.24 mL, 1.374 mmol, 3.0 eq) and piperidine (42.97 mg, 0.504 mmol, 1.1 eq) were added. The resulting reaction mass was stirred at RT for 16 h. The progress of the reaction was monitored by TLC and LCMS. After completion of the reaction, reaction mixture was concentrated under reduced pressure to afford crude which was diluted with water (50 mL), and product was extracted by EtOAc (2×75 mL). Combined organic layers were dried over anhydrous Na₂SO₄, filtered and concentrated under reduced pressure to afford (4-bromo-3-fluorophenyl)(piperidin-1-yl)methanone (120 mg, 92.3%), LCMS: 286.2[M+1]⁺.

### Step-2: Synthesis of (3-fluoro-4-(4,4,5,5-tetramethyl-1,3,2-dioxaborolan-2-yl)phenyl)(piperidin-1-yl)methanone

To a stirred solution of (4-bromo-3-fluorophenyl)(piperidin-1-yl)methanone (100 mg, 0.35 mmol, 1.0 eq) and Bis(pinacolato)diboron (97.59 mg, 0.38 mmol, 1.1 eq) in 1,4-Dioxane (4 mL), was added KOAc (104.1 mg, 1.05 mmol, 3.0 eq). The reaction mixture was purged with N₂ for 10 minutes, was added Pd(dppf)Cl₂·DCM (14.3 mg, 0.0175 mmol, 0.05 eq). The resulting reaction mixture was purged with N₂ for 10 minutes and stirred at 100° C. for 16 h. The progress of the reaction was monitored by LCMS and TLC. After completion of the reaction, reaction mixture was diluted with water (50 mL) and was extracted with EtOAc (2×75 mL). Combined organic layers were dried over Na₂SO₄, filtered and concentrated under reduced pressure to afford (3-fluoro-4-(4,4,5,5-tetramethyl-1,3,2-dioxaborolan-2-yl)phenyl)(piperidin-1-yl)methanone (110 mg, 94.8%), LCMS: 334.2[M+1]⁺.

### Step-3: Synthesis of (4-(4-chloroquinolin-6-yl)-3-fluorophenyl)(piperidin-1-yl)methanone

To a stirred solution of 6-bromo-4-chloroquinoline (65 mg, 0.268 mmol, 1.0 eq) and (3-fluoro-4-(4,4,5,5-tetramethyl-1,3,2-dioxaborolan-2-yl)phenyl)(piperidin-1-yl)methanone (107.1 mg, 0.321 mmol, 1.2 eq) in 1,4-dioxane:water (3 mL:0.75 mL), was added K₂CO₃ (55.5 mg, 0.402 mmol, 1.5 eq). Reaction mixture was purged with N2 gas for 10 minutes and was added Pd(dppf)Cl2·DCM (10.9 mg, 0.0134 mmol, 0.05 eq). Reaction mixture was stirred at 90° C. for 16 h. The progress of the reaction was monitored by LCMS and TLC. After completion of the reaction, reaction mixture was diluted with water (30 mL) and was extracted with EtOAc (2×50 mL), combined organic layers were dried over Na2SO4, filtered and concentrated under reduced pressure to afford crude which was purified by flash chromatography (eluted in 2% MeOH:DCM) to afford (4-(4-chloroquinolin-6-yl)-3-fluorophenyl)(piperidin-1-yl)methanone (50 mg, 50.5%), LCMS: 369.3 [M+1]⁺.

### Step-4: Synthesis of (4-(4-(benzo[d]thiazol-5-ylamino)quinolin-6-yl)-3-fluorophenyl)(piperidin-1-yl)methanone

To a solution of (4-(4-chloroquinolin-6-yl)-3-fluorophenyl)(piperidin-1-yl)methanone (50 mg, 0.135 mmol, 1.0 eq) and benzo[d]thiazol-5-amine (20.4 mg, 0.135 mmol, 1.0 eq) in EtOH (2 mL) was added TFA (0.05 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (25 mL) and was extracted with DCM (2×50 mL). Combined organic layers were dried over Na2SO4, filtered and concentrated under reduced pressure to afford which was purified by flash chromatography (eluted in 6% MeOH:DCM) to afford (4-(4-(benzo[d]thiazol-5-ylamino)quinolin-6-yl)-3-fluorophenyl)(piperidin-1-yl)methanone (16.88 mg, 26.1%). ANALYTICAL DATA: LCMS: 483.2 [M+1]⁺, ¹H NMR (400 MHz, DMSO-d₆) δ ppm 9.45 (s, 1H) 8.70 (s, 1H) 8.53 (d, J=5.72 Hz, 1H) 8.24 (d, J=8.58 Hz, 1H) 8.07 (d, J=1.91 Hz, 1H) 7.98-8.01 (m, 2H) 7.80 (s, 1H) 7.56 (dd, J=8.58, 1.91 Hz, 2H) 7.39-7.43 (m, 2H) 7.04 (d, J=5.72 Hz, 1H) 3.67 (br. s., 4H) 1.63 (br. s., 6H).

### Example 50. Synthesis of N-(7-(2-fluoro-4-(pyrrolidin-1-ylmethyl)phenyl)quinolin-4-yl)benzo[d]thiazol-5-amine (Compound 54)

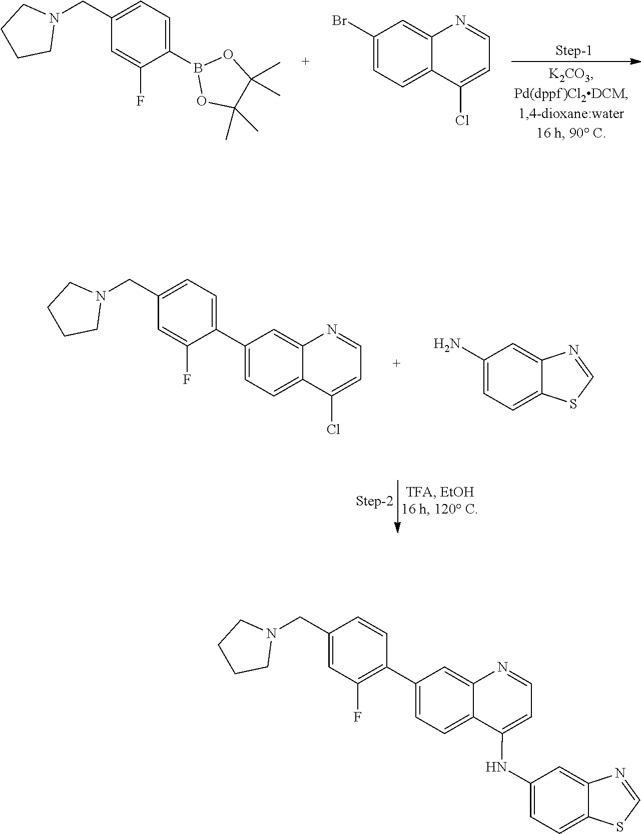 [SMILES: CC1(C)OB(c2ccc(CN3CCCC3)cc2F)OC1(C)C ; Clc1ccnc2cc(Br)ccc12 ; Fc1cc(CN2CCCC2)ccc1-c1ccc2c(Cl)ccnc2c1 ; Nc1ccc2scnc2c1 ; Fc1cc(CN2CCCC2)ccc1-c1ccc2c(Nc3ccc4scnc4c3)ccnc2c1]

### Step-1: Synthesis of 4-chloro-7-(2-fluoro-4-(pyrrolidin-1-ylmethyl)phenyl)quinoline

To a stirred solution of 7-bromo-4-chloroquinoline (100 mg, 0.412 mmol, 1.0 eq) and 1-(3-fluoro-4-(4,4,5,5-tetramethyl-1,3,2-dioxaborolan-2-yl)benzyl)pyrrolidine (125.8 mg, 0.412 mmol, 1.0 eq) in 1,4-dioxane:water (3 mL:0.5 mL), was added K₂CO₃ (85.2 mg, 0.618 mmol, 1.5 eq). Reaction mixture was purged with N₂ gas for 10 minutes and was added Pd(dppf)Cl₂·DCM (16.9 mg, 0.0206 mmol, 0.05 eq). Reaction mixture was stirred at 90° C. for 16 h. The progress of the reaction was monitored by LCMS and TLC. After completion of the reaction, reaction mixture was diluted with water (50 mL) and extracted with EtOAc (2×75 mL), combined organic layers were dried over Na₂SO₄, filtered and concentrated under reduced pressure to afford crude which was purified by flash chromatography (eluted in 2% MeOH:DCM) to afford 4-chloro-7-(2-fluoro-4-(pyrrolidin-1-ylmethyl)phenyl)quinoline (50 mg, 35.4%), LCMS: 341.3 [M+1]⁺.

### Step-2: Synthesis N-(7-(2-fluoro-4-(pyrrolidin-1-ylmethyl)phenyl)quinolin-4-yl)benzo[d]thiazol-5-amine

To a solution of 4-chloro-7-(2-fluoro-4-(pyrrolidin-1-ylmethyl)phenyl)quinoline (50 mg, 0.146 mmol, 1.0 eq) and benzo[d]thiazol-5-amine (22.1 mg, 0.146 mmol, 1.0 eq) in EtOH (2 mL) was added TFA (0.03 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (25 mL) and was extracted with DCM (2×50 mL). Combined organic layers were dried over Na₂SO₄, filtered and concentrated under reduced pressure to afford which was purified by flash chromatography (eluted in 6% MeOH:DCM) to afford N-(7-(2-fluoro-4-(pyrrolidin-1-ylmethyl)phenyl)quinolin-4-yl)benzo[d]thiazol-5-amine (13.1 mg, 19.6%), LCMS: 455.3 [M+1]⁺, ¹H NMR (400 MHz, DMSO-d₆) δ ppm 9.43 (s, 1H) 9.29 (br. s., 1H) 8.61 (s, 1H) 8.51 (d, J=5.25 Hz, 1H) 8.20 (d, J=8.58 Hz, 1H) 8.03 (d, J=1.91 Hz, 1H) 7.97 (d, J=8.58 Hz, 1H) 7.89 (d, J=9.06 Hz, 1H) 7.68 (t, J=8.11 Hz, 1H) 7.55 (dd, J=8.58, 1.91 Hz, 1H) 7.25-7.33 (m, 2H) 7.05 (d, J=5.25 Hz, 1H) 3.61 (s, 2H) 3.54 (br. s., 4H) 1.50-1.57 (m, 4H).

### Example 51. Synthesis of N-(6-(2-fluoro-5-(piperidin-1-ylmethyl)phenyl)quinolin-4-yl)benzo[d]thiazol-5-amine (Compound 55)

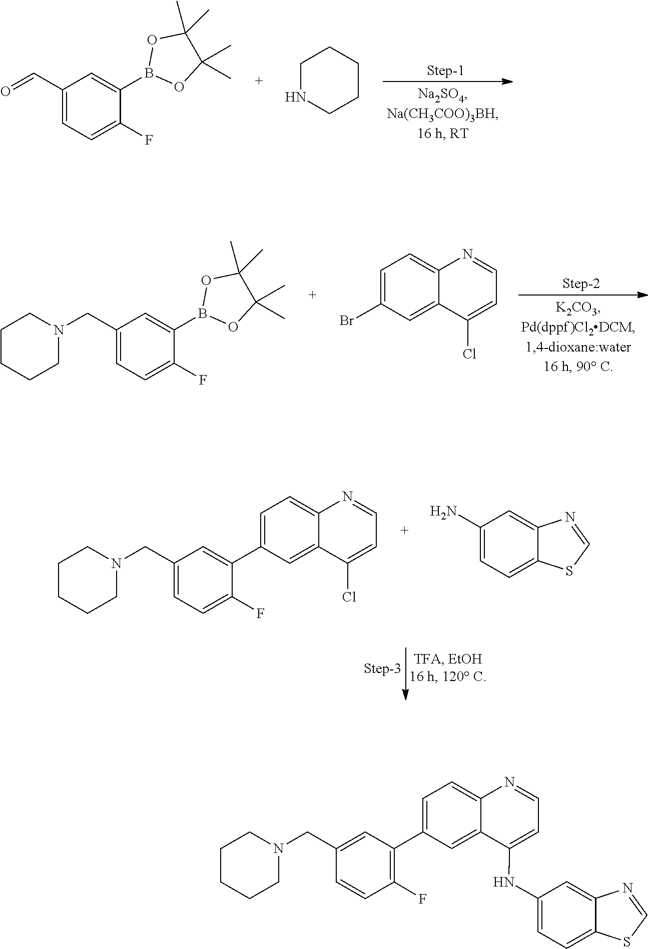

### Step-1: Synthesis of 1-(4-fluoro-3-(4,4,5,5-tetramethyl-1,3,2-dioxaborolan-2-yl)benzyl)piperidine

To a stirred solution of 4-fluoro-3-(4,4,5,5-tetramethyl-1,3,2-dioxaborolan-2-yl)benzaldehyde (300 mg, 1.2 mmol, s1 eq) and piperidine (102.19 mg, 1.2 mmol, 1.0 eq) in DCM (15 mL), was added Na₂SO₄ (338 mg, 2.4 mmol, 2.0 eq). Reaction mixture was stirred at RT for 1 h. Further, was added sodium triacetoxyborohydride (378 mg, 1.8 mmol, 1.5 eq) portion wise at 0° C. After addition, reaction mixture was stirred at RT for 16 h. The progress of the reaction was monitored by LCMS. After completion of the reaction, reaction mixture was diluted with water (75 mL) and was extracted with DCM (2×100 mL). Combined organic layers were washed with saturated NaHCO₃ solution (50 mL), Organic layer was dried over anhydrous Na₂SO₄, filtered and concentrated under reduced pressure to afford 1-(4-fluoro-3-(4,4,5,5-tetramethyl-1,3,2-dioxaborolan-2-yl)benzyl)piperidine (310 mg, 81.00%), LCMS: 238.13 [Boronic-acid Fragment].

### Step-2: Synthesis of 4-chloro-6-(2-fluoro-5-(piperidin-1-ylmethyl)phenyl)quinoline

To a stirred solution of 6-bromo-4-chloroquinoline (100 mg, 0.412 mmol, 1.0 eq) and 1-(4-fluoro-3-(4,4,5,5-tetramethyl-1,3,2-dioxaborolan-2-yl)benzyl)piperidine (158 mg, 0.494 mmol, 1.2 eq) in 1,4-dioxane:water (2.5 mL:0.5 mL), was added K₂CO₃ (0.86 mg, 0.618 mmol, 1.5 eq). Reaction mixture was purged with N2 gas for 10 minutes and was added Pd(dppf)Cl2·DCM (0.17 mg, 0.0206 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford 4-chloro-6-(2-fluoro-5-(piperidin-1-ylmethyl)phenyl)quinoline (50 mg, 34.24%), LCMS: 355.3 [M+1]⁺.

### Step-3: Synthesis of N-(6-(2-fluoro-5-(piperidin-1-ylmethyl)phenyl)quinolin-4-yl)benzo[d]thiazol-5-amine

To a solution of 4-chloro-6-(2-fluoro-5-(piperidin-1-ylmethyl)phenyl)quinoline (80 mg, 0.225 mmol, 1.0 eq) and benzo[d]thiazol-5-amine (33.8 mg, 0.225 mmol, 1.0 eq) in EtOH (6 mL) was added TFA (0.08 mL) and reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (30 mL) and was extracted with DCM (2×50 mL). Combined organic layers were dried over Na₂SO₄, filtered and concentrated under reduced pressure to afford which was purified by flash chromatography (eluted in 6% MeOH:DCM) to afford N-(6-(2-fluoro-5-(piperidin-1-ylmethyl)phenyl)quinolin-4-yl)benzo[d]thiazol-5-amine (6.2 mg, 5.8%), LCMS: 469.3 [M+1]⁺, ¹H NMR (400 MHz, DMSO-d₆) δ ppm 9.43 (s, 1H) 9.29 (br. s., 1H) 8.61 (s, 1H) 8.51 (d, J=5.25 Hz, 1H) 8.20 (d, J=8.58 Hz, 1H) 8.03 (d, J=1.91 Hz, 1H) 7.97 (d, J=8.58 Hz, 1H) 7.89 (d, J=9.06 Hz, 1H) 7.68 (t, J=8.11 Hz, 1H) 7.55 (dd, J=8.58, 1.91 Hz, 1H) 7.25-7.33 (m, 2H) 7.05 (d, J=5.25 Hz, 1H) 3.61 (s, 2H) 3.54 (br. s., 4H) 1.50-1.57 (m, 4H) 1.41 (br. s., 2H).

### Example 52. Synthesis of N-(7-(2-fluoro-5-(piperidin-1-ylmethyl)phenyl)quinolin-4-yl)benzo[d]thiazol-5-amine (Compound 56)

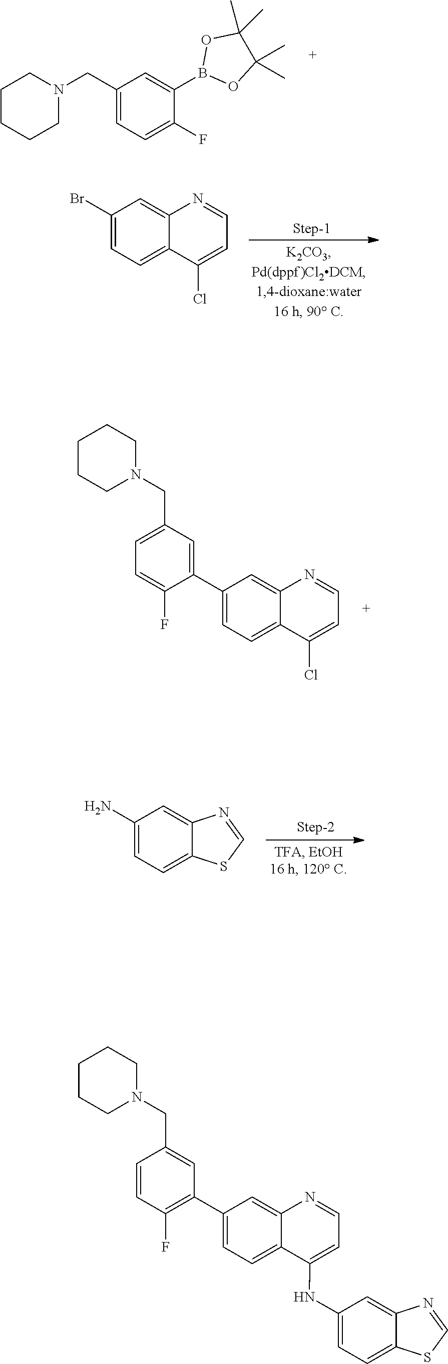 [SMILES: CC1(C)OB(c2cc(CN3CCCCC3)ccc2F)OC1(C)C ; Clc1ccnc2cc(Br)ccc12 ; Fc1ccc(CN2CCCCC2)cc1-c1ccc2c(Cl)ccnc2c1 ; Nc1ccc2scnc2c1 ; Fc1ccc(CN2CCCCC2)cc1-c1ccc2c(Nc3ccc4scnc4c3)ccnc2c1]

### Step-1: Synthesis of 4-chloro-7-(2-fluoro-5-(piperidin-1-ylmethyl)phenyl)quinoline

To a stirred solution of 7-bromo-4-chloroquinoline (100 mg, 0.412 mmol, 1.0 eq) and 1-(4-fluoro-3-(4,4,5,5-tetramethyl-1,3,2-dioxaborolan-2-yl)benzyl)piperidine (0.158 mg, 0.494 mmol, 1.2 eq) in 1,4-dioxane:water (2.5 mL:0.5 mL), was added K₂CO₃ (0.86 mg, 0.618 mmol, 1.5 eq). Reaction mixture was purged with N2 gas for 10 minutes and was added Pd(dppf)Cl2·DCM (0.17 mg, 0.0206 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford 4-chloro-7-(2-fluoro-5-(piperidin-1-ylmethyl)phenyl)quinoline (60 mg, 41.3%), LCMS: 355.3 [M+1]⁺.

### Step-2: Synthesis of N-(7-(2-fluoro-5-(piperidin-1-ylmethyl)phenyl)quinolin-4-yl)benzo[d]thiazol-5-amine

To a solution of 4-chloro-7-(2-fluoro-5-(piperidin-1-ylmethyl)phenyl)quinoline (30 mg, 0.0847 mmol, 1.0 eq) and benzo[d]thiazol-5-amine (12.71 mg, 0.0847 mmol, 1.0 eq) in EtOH (2 mL) was added TFA (0.03 mL) and the reaction mixture was stirred at 120° C. for 16 h. The progress of reaction was monitored by TLC and LCMS. After completion of the reaction, reaction mixture was concentrated under reduced pressure to obtain crude which was neutralized with saturated NaHCO₃ solution (30 mL) and was extracted with DCM (2×50 mL). Combined organic layers were dried over Na₂SO₄, filtered and concentrated under reduced pressure to afford which was purified by flash chromatography (eluted in 6% MeOH DCM) to afford N-(7-(2-fluoro-5-(piperidin-1-ylmethyl)phenyl)quinolin-4-yl)benzo[d]thiazol-5-amine (6.2 mg, 15.3%), LCMS: 469.3 [M+1]⁺, ¹H NMR (400 MHz, DMSO-d₆) δ ppm 9.43 (s, 1H) 9.29 (br. s., 1H) 8.61 (s, 1H) 8.51 (d, J=5.25 Hz, 1H) 8.20 (d, J=8.58 Hz, 1H) 8.03 (d, J=1.91 Hz, 1H) 7.97 (d, J=8.58 Hz, 1H) 7.89 (d, J=9.06 Hz, 1H) 7.68 (t, J=8.11 Hz, 1H) 7.55 (dd, J=8.58, 1.91 Hz, 1H) 7.24-7.30 (m, 2H) 7.1 (d, J=5.25 Hz, 1H) 3.51 (s, 2H) 3.42 (br. s., 4H) 1.49-1.57 (m, 4H) 1.35 (br. s., 2H).

### Example 53. Synthesis of tert-butyl 3-(4-(4-(benzo[d]thiazol-5-ylamino)quinolin-6-yl)-3-fluorobenzyl)-3,8-diazabicyclo[3.2.1]octane-8-carboxylate (Compound 57)

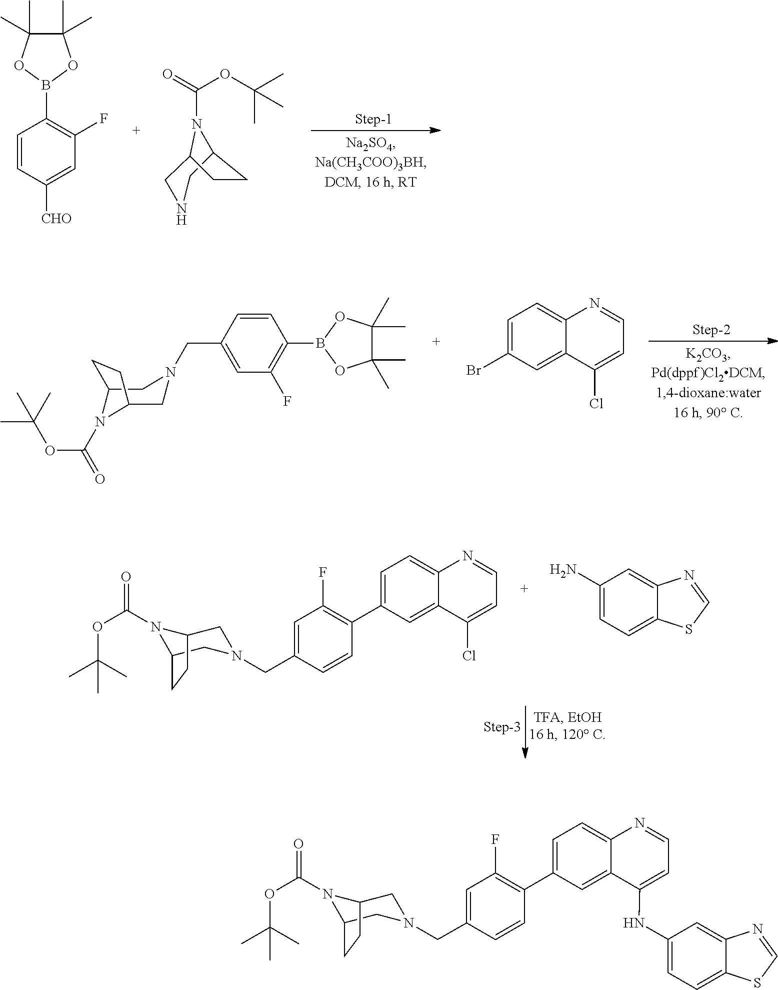

### Step-1: Synthesis of tert-butyl 3-(3-fluoro-4-(4,4,5,5-tetramethyl-1,3,2-dioxaborolan-2-yl)benzyl)-3,8-diazabicyclo[3.2.1]octane-8-carboxylate

To a stirred solution of 3-fluoro-4-(4,4,5,5-tetramethyl-1,3,2-dioxaborolan-2-yl)benzaldehyde (400 mg, 1.6 mmol, 1 eq) and tert-butyl 3,8-diazabicyclo[3.2.1]octane-8-carboxylate (339.2 mg, 1.6 mmol, 1.0 eq) in DCM (20 mL), was added Na₂SO₄ (454.8 mg, 3.2 mmol, 2.0 eq). Reaction mixture was stirred at RT for 1 h. Further, was added sodium triacetoxyborohydride (508.6 mg, 2.4 mmol, 1.5 eq) portion wise at 0° C. After addition, reaction mixture was stirred at RT for 16 h. The progress of the reaction was monitored by LCMS. After completion of the reaction, reaction mixture was diluted with water (75 mL) and was extracted with DCM (2×100 mL). Combined organic layers were washed with saturated NaHCO₃ solution (50 mL), Organic layer was dried over anhydrous Na₂SO₄, filtered and concentrated under reduced pressure to afford tert-butyl 3-(3-fluoro-4-(4,4,5,5-tetramethyl-1,3,2-dioxaborolan-2-yl)benzyl)-3,8-diazabicyclo[3.2.1]octane-8-carboxylate (300 mg, 42.07%), LCMS: 365.4[M+1]⁺(Boronic acid Fragment).

### Step-2: Synthesis of tert-butyl 3-(4-(4-chloroquinolin-6-yl)-3-fluorobenzyl)-3,8-diazabicyclo[3.2.1]octane-8-carboxylate

To a stirred solution of 6-bromo-4-chloroquinoline (150 mg, 0.619 mmol, 1.0 eq) and tert-butyl 3-(3-fluoro-4-(4,4,5,5-tetramethyl-1,3,2-dioxaborolan-2-yl)benzyl)-3,8-diazabicyclo[3.2.1]octane-8-carboxylate (331.7 mg, 0.743 mmol, 1.2 eq) in 1,4-dioxane: water (5 mL:1 mL), was added K₂CO₃ (128.13 mg, 0.928 mmol, 1.5 eq). Reaction mixture was purged with N2 gas for 10 minutes and was added Pd(dppf)Cl2·DCM (25.27 mg, 0.030 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford tert-butyl 3-(4-(4-chloroquinolin-6-yl)-3-fluorobenzyl)-3,8-diazabicyclo[3.2.1]octane-8-carboxylate (68 mg, 41.9%). LCMS: 482.3 [M+1]⁺.

### Step-3: Synthesis of tert-butyl 3-(4-(4-(benzo[d]thiazol-5-ylamino)quinolin-6-yl)-3-fluorobenzyl)-3,8-diazabicyclo[3.2.1]octane-8-carboxylate

To a solution of tert-butyl 3-(4-(4-chloroquinolin-6-yl)-3-fluorobenzyl)-3,8-diazabicyclo[3.2.1]octane-8-carboxylate (68 mg, 0.141 mmol, 1.0 eq) and benzo[d]thiazol-5-amine (21.23 mg, 0.141 mmol, 1.0 eq) in EtOH (4 mL) was added TFA (0.08 mL) and the reaction mixture was stirred at 120° C. for 16 h. the progress of the reaction was monitored by TLC and LCMS. After completion of the reaction, reaction mixture was concentrated under reduced pressure to obtain crude which was neutralized with saturated NaHCO₃ solution (30 mL) and was extracted with DCM (2×50 mL). Combined organic layers were dried over Na₂SO₄, filtered and concentrated under reduced pressure to afford which was purified by flash chromatography (eluted in 6% MeOH:DCM) to afford tert-butyl 3-(4-(4-(benzo[d]thiazol-5-ylamino)quinolin-6-yl)-3-fluorobenzyl)-3,8-diazabicyclo[3.2.1]octane-8-carboxylate (41 mg, 48.80%), LCMS: 596.3 [M+1]⁺, ¹H NMR (400 MHz, DMSO-d₆) δ ppm 9.43 (s, 1H) 9.24 (s, 1H) 8.43-8.55 (m, 2H) 8.20 (d, J=8.58 Hz, 1H) 8.05 (s, 2H) 7.68-7.79 (m, 2H) 7.56 (dd, J=8.58, 1.91 Hz, 1H) 7.29-7.34 (m, 2H) 7.04 (d, J=5.25 Hz, 1H) 4.05 (br. s., 2H) 3.55 (s, 2H) 2.65 (d, J=10.49 Hz, 2H) 2.22 (d, J=10.49 Hz, 2H) 1.87 (d, J=6.20 Hz, 2H) 1.78 (br. s., 2H) 1.40 (s, 9H).

### Example 54. Synthesis of tert-butyl 3-(4-(4-(benzo[d]thiazol-5-ylamino)quinolin-7-yl)-3-fluorobenzyl)-3,8-diazabicyclo[3.2.1]octane-8-carboxylate (Compound 58)

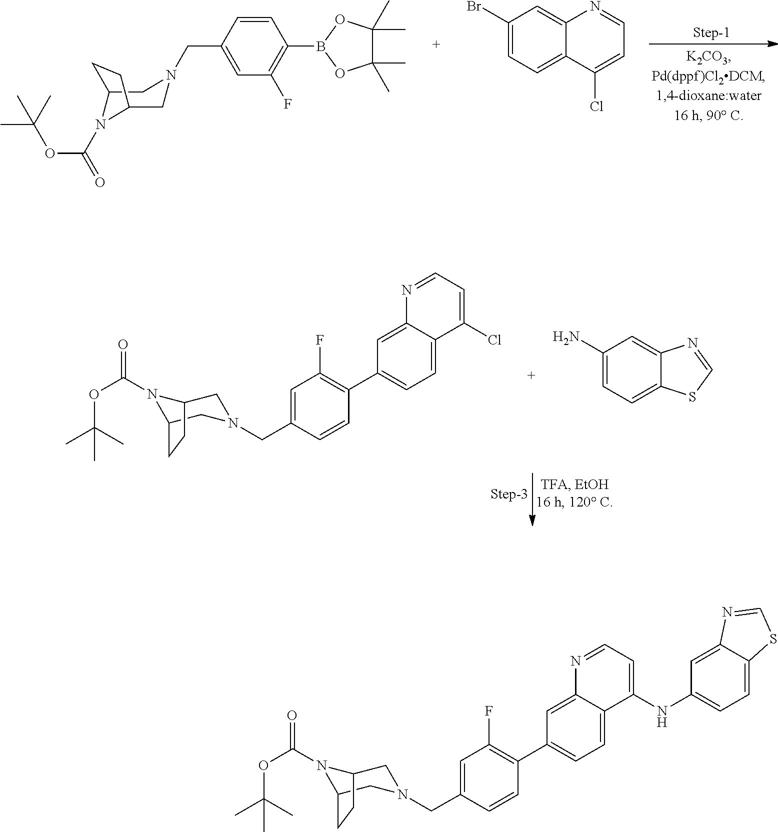 [SMILES: CC(C)(C)OC(=O)N1C2CCC1CN(Cc1ccc(B3OC(C)(C)C(C)(C)O3)c(F)c1)C2 ; Clc1ccnc2cc(Br)ccc12 ; CC(C)(C)OC(=O)N1C2CCC1CN(Cc1ccc(-c3ccc4c(Cl)ccnc4c3)c(F)c1)C2 ; Nc1ccc2scnc2c1 ; CC(C)(C)OC(=O)N1C2CCC1CN(Cc1ccc(-c3ccc4c(Nc5ccc6scnc6c5)ccnc4c3)c(F)c1)C2]

### Step-1: Synthesis of tert-butyl 3-(4-(4-chloroquinolin-7-yl)-3-fluorobenzyl)-3,8-diazabicyclo[3.2.1]octane-8-carboxylate

To a stirred solution of 7-bromo-4-chloroquinoline (150 mg, 0.618 mmol, 1.0 eq) and tert-butyl 3-(3-fluoro-4-(4,4,5,5-tetramethyl-1,3,2-dioxaborolan-2-yl)benzyl)-3,8-diazabicyclo[3.2.1]octane-8-carboxylate (331.05 mg, 0.742 mmol, 1.2 eq) in s1,4-dioxane:water (5 mL:1 mL), was added K₂CO₃ (128 mg, 0.9297 mmol, 1.5 eq). Reaction mixture was purged with N2 gas for 10 minutes and was added Pd(dppf)Cl2·DCM (25.2 mg, 0.0309 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford tert-butyl 3-(4-(4-chloroquinolin-7-yl)-3-fluorobenzyl)-3,8-diazabicyclo[3.2.1]octane-8-carboxylate (80 mg, 49.3%). LCMS: 482.3[M+1]⁺.

### Step-2: Synthesis of tert-butyl 3-(4-(4-(benzo[d]thiazol-5-ylamino)quinolin-7-yl)-3-fluorobenzyl)-3,8-diazabicyclo[3.2.1]octane-8-carboxylate

To a solution of tert-butyl 3-(4-(4-chloroquinolin-7-yl)-3-fluorobenzyl)-3,8-diazabicyclo[3.2.1]octane-8-carboxylate (80 mg, 0.166 mmol, 1.0 eq) and benzo[d]thiazol-5-amine (24.9 mg, 0.166 mmol, 1.0 eq) in EtOH (4 mL) was added TFA (0.075 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (30 mL) and was extracted with DCM (2×50 mL). Combined organic layers were dried over Na2SO4, filtered and concentrated under reduced pressure to afford which was purified by flash chromatography (eluted in 6% MeOH:DCM) to afford tert-butyl 3-(4-(4-(benzo[d]thiazol-5-ylamino)quinolin-7-yl)-3-fluorobenzyl)-3,8-diazabicyclo[3.2.1]octane-8-carboxylate (14 mg, 14.2%), LCMS: 596.3 [M+1]⁺, ¹H NMR (400 MHz, DMSO-d₆) δ ppm 9.43 (s, 1H) 9.24 (s, 1H) 8.43-8.55 (m, 2H) 8.20 (d, J=8.58 Hz, 1H) 8.05 (s, 2H) 7.68-7.79 (m, 2H) 7.56 (dd, J=8.58, 1.91 Hz, 1H) 7.29-7.34 (m, 2H) 7.04 (d, J=5.25 Hz, 1H) 4.05 (br. s., 2H) 3.55 (s, 2H) 2.65 (d, J=10.49 Hz, 2H) 2.22 (d, J=10.49 Hz, 2H) 1.87 (d, J=6.20 Hz, 2H) 1.78 (br. s., 2H) 1.40 (s, 9H).

### Example 55. Synthesis of (4-(4-(benzo[d]thiazol-5-ylamino)quinolin-7-yl)-3-fluorophenyl)(piperidin-1-yl)methanone (Compound 61)

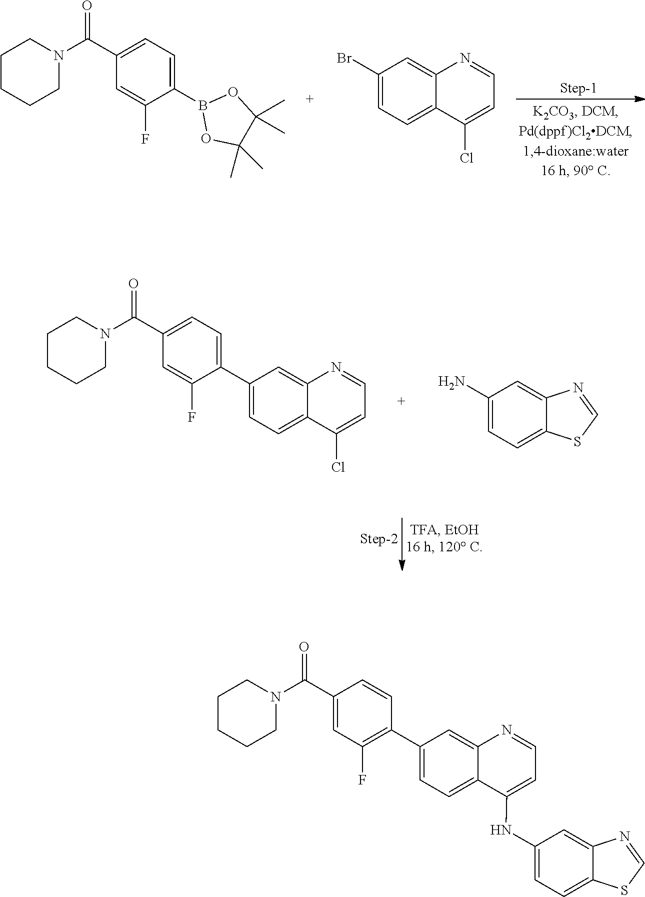 [SMILES: CC1(C)OB(c2ccc(C(=O)N3CCCCC3)cc2F)OC1(C)C ; Clc1ccnc2cc(Br)ccc12 ; O=C(c1ccc(-c2ccc3c(Cl)ccnc3c2)c(F)c1)N1CCCCC1 ; Nc1ccc2scnc2c1 ; O=C(c1ccc(-c2ccc3c(Nc4ccc5scnc5c4)ccnc3c2)c(F)c1)N1CCCCC1]

### Step-1: Synthesis of (4-(4-chloroquinolin-7-yl)-3-fluorophenyl)(piperidin-1-yl)methanone

To a stirred solution of 7-bromo-4-chloroquinoline (110 mg, 0.453 mmol, 1.0 eq) and (3-fluoro-4-(4,4,5,5-tetramethyl-1,3,2-dioxaborolan-2-yl)phenyl)(piperidin-1-yl)methanone (181.3 mg, 0.544 mmol, 1.2 eq) in 1,4-dioxane:water (5 mL:1 mL), was added K₂CO₃ (93.9 mg, 0.679 mmol, 1.5 eq). Reaction mixture was purged with N2 gas for 10 minutes and was added Pd(dppf)Cl2·DCM (18.5 mg, 0.022 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×100 mL), combined organic layers were dried over Na₂SO₄, filtered and concentrated under reduced pressure to afford crude which was purified by flash chromatography (eluted in 2% MeOH:DCM) to afford (4-(4-chloroquinolin-7-yl)-3-fluorophenyl)(piperidin-1-yl)methanone ((80 mg, 47.6%), LCMS: 369.3 [M+1]⁺.

### Step-2: Synthesis of (4-(4-(benzo[d]thiazol-5-ylamino)quinolin-7-yl)-3-fluorophenyl)(piperidin-1-yl)methanone

To a solution of (4-(4-chloroquinolin-7-yl)-3-fluorophenyl)(piperidin-1-yl)methanone (50 mg, 0.135 mmol, 1.0 eq) and benzo[d]thiazol-5-amine (20.4 mg, 0.135 mmol, 1.0 eq) in EtOH (2 mL) was added TFA (0.05 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (25 mL) and was extracted with DCM (2×50 mL). Combined organic layers were dried over Na2SO4, filtered and concentrated under reduced pressure to afford which was purified by flash chromatography (eluted in 6% MeOH:DCM) to afford (4-(4-(benzo[d]thiazol-5-ylamino)quinolin-7-yl)-3-fluorophenyl)(piperidin-1-yl)methanone (33 mg, 50.76%), LCMS: 483.3 [M+1]⁺, ¹H NMR (400 MHz, DMSO-d₆) δ ppm 9.45 (s, 1H) 8.70 (s, 1H) 8.53 (d, J=5.72 Hz, 1H) 8.24 (d, J=8.58 Hz, 1H) 8.07 (d, J=1.91 Hz, 1H) 7.98-8.01 (m, 2H) 7.80 (s, 1H) 7.56 (dd, J=8.58, 1.91 Hz, 2H) 7.39-7.43 (m, 2H) 7.04 (d, J=5.72 Hz, 1H) 3.67 (br. s., 4H) 1.63 (br. s., 6H).

### Example 56. Synthesis of 1-(4-(4-(4-(benzo[d]thiazol-5-ylamino)quinolin-6-yl)phenyl)piperidin-1-yl)-2-hydroxyethan-1-one (Compound 239)

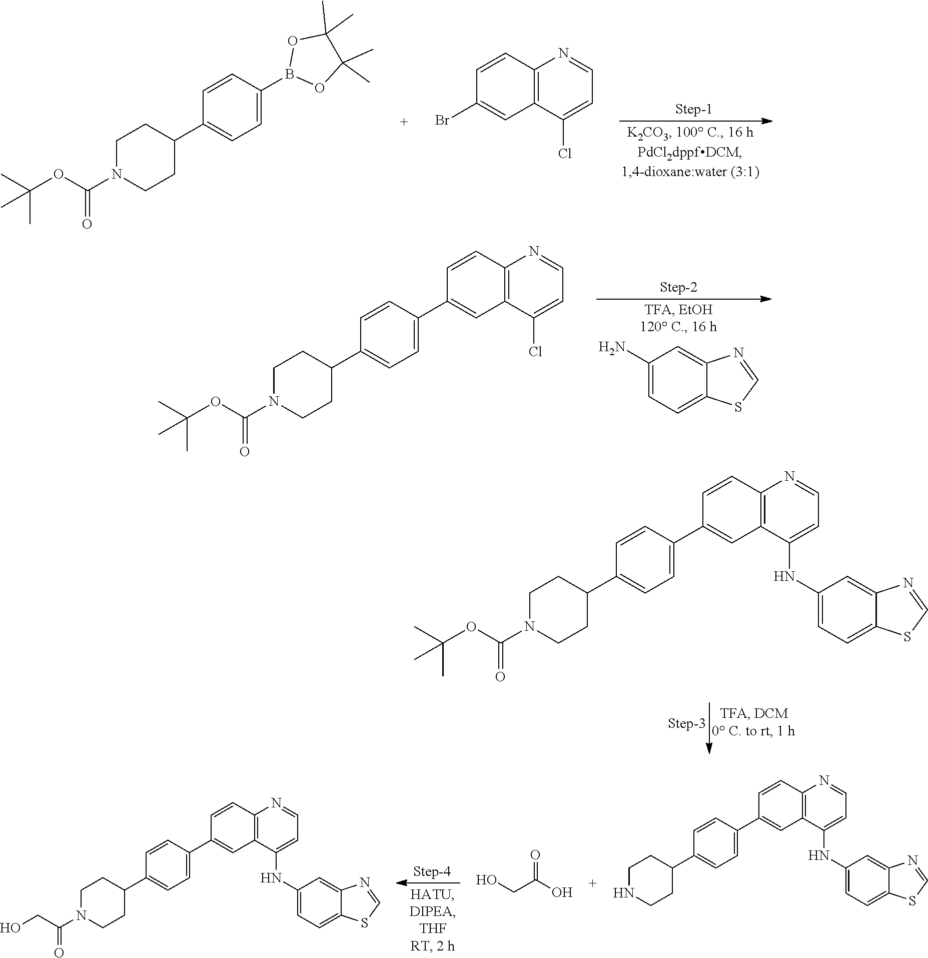

### Step-1: Synthesis of tert-butyl 4-(4-(4-chloroquinolin-6-yl)phenyl)piperidine-1-carboxylate

To a solution tert-butyl 4-(4-(4,4,5,5-tetramethyl-1,3,2-dioxaborolan-2-yl)phenyl)piperidine-1-carboxylate (150 mg, 0.387 mmol, 1 eq) in 1,4-dioxane:water (3.5 mL:1.5 mL) was added K₂CO₃ (107 mg, 0.774 mmol, 2 eq) and 6-bromo-4-chloroquinoline (93 mg, 0.387 mmol, 1 eq). Reaction mixture was purged with nitrogen for 5 min, was added PdCl₂dppf·DCM (15 mg, 0.019 mmol, 0.05 eq). The reaction mixture was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under vacuum to afford crude which was purified by flash chromatography to afford tert-butyl 4-(4-(4-chloroquinolin-6-yl)phenyl)piperidine-1-carboxylate (160 mg, 98.1%), LCMS: 423.4[M+1]⁺.

### Step-2: Synthesis of tert-butyl 4-(4-(4-(benzo[d]thiazol-5-ylamino)quinolin-6-yl)phenyl)piperidine-1-carboxylate

To a stirred solution of tert-butyl 4-(4-(4-chloroquinolin-6-yl)phenyl)piperidine-1-carboxylate (160 mg, 0.378 mmol, 1.0 eq) in ethanol (5 mL) was added benzo[d]thiazol-5-amine (85 mg, 0.567 mmol, 1.5 eq) and TFA (0.1 mL). After addition, the reaction mixture was heated at 120° C. for 16 h. After completion of the reaction, reaction mixture was concentrated under vacuum to obtain crude which was neutralized by saturated sodium bicarbonate solution (40 mL), was extracted with ethyl acetate (2×80 mL), dried over anhydrous sodium sulphate, filtered and concentrated under vacuum to afford crude which was purified by flash chromatography to afford tert-butyl 4-(4-(4-(benzo[d]thiazol-5-ylamino)quinolin-6-yl)phenyl)piperidine-1-carboxylate (200 mg, 98.5%), LCMS: 537.2[M+1]⁺.

### Step-3: Synthesis of N-(6-(4-(piperidin-4-yl)phenyl)quinolin-4-yl)benzo[d]thiazol-5-amine

To a stirred solution of tert-butyl 4-(4-(4-(benzo[d]thiazol-5-ylamino)quinolin-6-yl)phenyl)piperidine-1-carboxylate (200 mg, 0.372 mmol, 1.0 eq) in dichloromethane (10 mL) was added TFA (1 mL) at 0° C. The reaction mixture was stirred at RT for 1 h. After completion of reaction, reaction mixture was concentrated under vacuum to afford N-(6-(4-(piperidin-4-yl)phenyl)quinolin-4-yl)benzo[d]thiazol-5-amine (150 mg, 92.5%), LCMS: 437.1 [M+1]⁺.

### Step-4: Synthesis of 1-(4-(4-(4-(benzo[d]thiazol-5-ylamino)quinolin-6-yl)phenyl)piperidin-1-yl)-2-hydroxyethan-1-one

To a stirred solution of N-(6-(4-(piperidin-4-yl)phenyl)quinolin-4-yl)benzo[d]thiazol-5-amine (50 mg, 0.114 mmol, 1.0 eq) in THF (5 mL) was added DIPEA (0.04 mL), HATU (65 mg, 0.171 mmol) and 2-hydroxyacetic acid (10 mg, 0.137 mmol) at room temperature. After addition, the reaction mixture was stirred at RT for 2 h. After completion of reaction, reaction mixture was diluted with water (10 mL), was extracted with ethyl acetate (2×20 mL), dried over anhydrous sodium sulphate, filtered and concentrated under vacuum to afford crude which was purified by flash chromatography to afford 1-(4-(4-(4-(benzo[d]thiazol-5-ylamino)quinolin-6-yl)phenyl)piperidin-1-yl)-2-hydroxyethan-1-one (10 mg, 17.8%), LCMS: 495.2[M+1]⁺, ¹H NMR (400 MHz, DMSO-d₆) δ ppm 1.61 (m, 2H) 1.76 (m, 2H) 2.60-2.80 (m, 2H) 3.01-3.19 (m, 3H) 4.06-4.18 (m, 2H) 4.40-4.60 (m, 1H) 6.98 (d, J=5.72 Hz, 1H) 7.44 (m, J=8.27 Hz, 2H) 7.59 (d, J=8.27 Hz, 2H) 7.87 (m, J=7.63 Hz, 2H) 8.00 (d, J=8.90 Hz, 1H) 8.13 (br. s., 1H) 8.18 (br. s., 1H) 8.29 (d, J=8.90 Hz, 1H) 8.49 (d, J=6.36 Hz, 1H) 8.84 (br. s., 1H) 9.48 (s, 1H).

### Example 57. Synthesis of N-(6-(4-(piperidin-4-yl)phenyl)quinolin-4-yl)benzo[d]thiazol-5-amine (Compound 240)

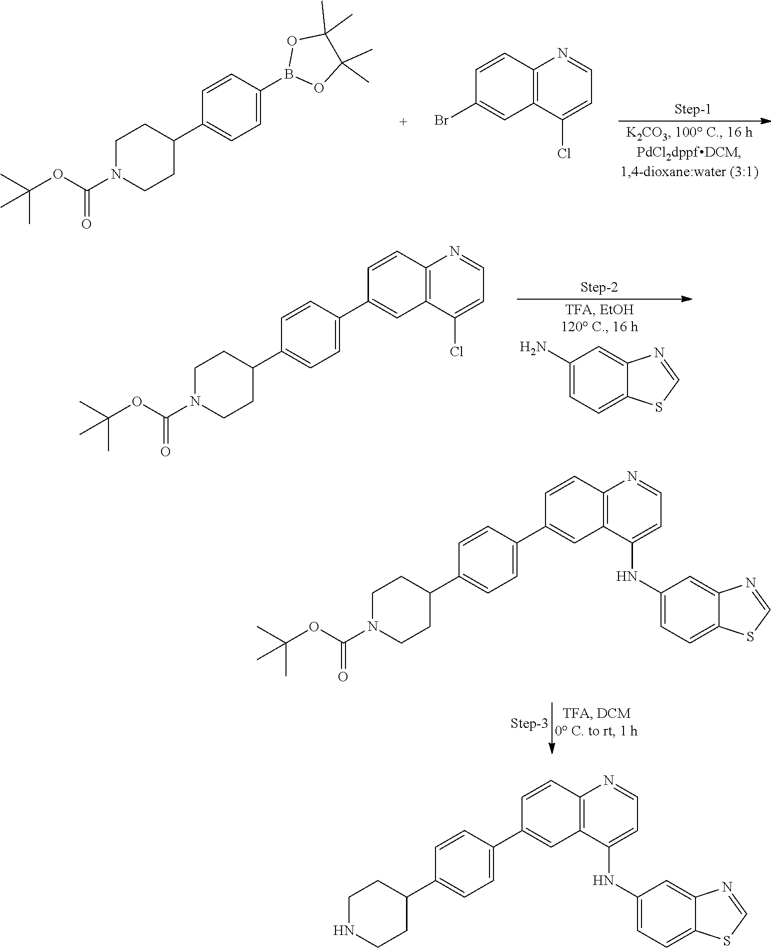 [SMILES: CC(C)(C)OC(=O)N1CCC(c2ccc(B3OC(C)(C)C(C)(C)O3)cc2)CC1 ; Clc1ccnc2ccc(Br)cc12 ; CC(C)(C)OC(=O)N1CCC(c2ccc(-c3ccc4nccc(Cl)c4c3)cc2)CC1 ; Nc1ccc2scnc2c1 ; CC(C)(C)OC(=O)N1CCC(c2ccc(-c3ccc4nccc(Nc5ccc6scnc6c5)c4c3)cc2)CC1 ; c1cc(Nc2ccc3scnc3c2)c2cc(-c3ccc(C4CCNCC4)cc3)ccc2n1]

### Step-1: Synthesis of tert-butyl 4-(4-(4-chloroquinolin-6-yl)phenyl)piperidine--carboxylate

To a solution tert-butyl 4-(4-(4,4,5,5-tetramethyl-1,3,2-dioxaborolan-2-yl)phenyl)piperidine-1-carboxylate (150 mg, 0.387 mmol, 1 eq) in 1,4-dioxane:water (3.5 mL:1.5 mL) was added K₂CO₃ (107 mg, 0.774 mmol, 2 eq) and 6-bromo-4-chloroquinoline (93 mg, 0.387 mmol, 1 eq). Reaction mixture was purged with nitrogen for 5 m, was added PdCl₂dppf·DCM (15 mg, 0.019 mmol, 0.05 eq). The reaction mixture was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under vacuum to afford crude which was purified by flash chromatography to afford tert-butyl 4-(4-(4-chloroquinolin-6-yl)phenyl)piperidine-1-carboxylate (160 mg, 98.1%), LCMS:

### Step-2: Synthesis of tert-butyl 4-(4-(4-(benzo[d]thiazol-5-ylamino)quinolin-6-yl)phenyl)piperidine-1-carboxylate

To a stirred solution of tert-butyl 4-(4-(4-chloroquinolin-6-yl)phenyl)piperidine-1-carboxylate (160 mg, 0.378 mmol, 1.0 eq) in ethanol (5 mL) was added benzo[d]thiazol-5-amine (85 mg, 0.567 mmol, 1.5 eq) and TFA (0.1 mL). After addition, the reaction mixture was heated at 120° C. for 16 h. After completion of the reaction, reaction mixture was concentrated under vacuum to obtain crude which was neutralized by saturated sodium bicarbonate solution (40 mL), was extracted with ethyl acetate (2×80 mL), dried over anhydrous sodium sulphate, filtered and concentrated under vacuum to afford crude which was purified by flash chromatography to afford tert-butyl 4-(4-(4-(benzo[d]thiazol-5-ylamino)quinolin-6-yl)phenyl)piperidine-1-carboxylate (200 mg, 98.5%), LCMS: 537.2[M+1]⁺.

### Step-3: Synthesis of N-(6-(4-(piperidin-4-yl)phenyl)quinolin-4-yl)benzo[d]thiazol-5-amine

To a stirred solution of tert-butyl 4-(4-(4-(benzo[d]thiazol-5-ylamino)quinolin-6-yl)phenyl)piperidine-1-carboxylate (50 mg, 0.093 mmol, 1.0 eq) in dichloromethane (5 mL) was added TFA (0.2 mL) at 0° C. The reaction mixture was stirred at RT for 1 h. After completion of reaction, reaction mixture was concentrated under vacuum to afford crude neutralized by using saturated sodium bicarbonate solution and extracted with DCM, concentrated under vacuum to obtain crude which was triturated with diethyl ether to obtain N-(6-(4-(piperidin-4-yl)phenyl)quinolin-4-yl)benzo[d]thiazol-5-amine (22 mg, 54.09%), LCMS: 437.3[M+1]⁺. ¹H NMR (400 MHz, DMSO-d₆) δ ppm 1.58-1.65 (m, 2H) 1.78 (d, J=12.08 Hz, 2H) 2.64-2.70 (m, 3H) 3.10 (d, J=11.44 Hz, 2H) 7.02 (s, 1H) 7.39 (m, J=8.27 Hz, 2H) 7.57 (d, J=6.99 Hz, 1H) 7.85 (m, J=8.27 Hz, 2H) 7.93-7.97 (m, 1H) 8.02-8.06 (m, 2H) 8.21 (d, J=8.27 Hz, 1H) 8.47 (d, J=5.72 Hz, 1H) 8.69 (s, 1H) 9.28 (s, 1H) 9.43 (s, 1H).

### Example 58. Synthesis of (4-(4-(benzo[d]thiazol-5-ylamino)quinolin-6-yl)-3-fluorophenyl)(pyrrolidin-1-yl)methanone (Compound 241)

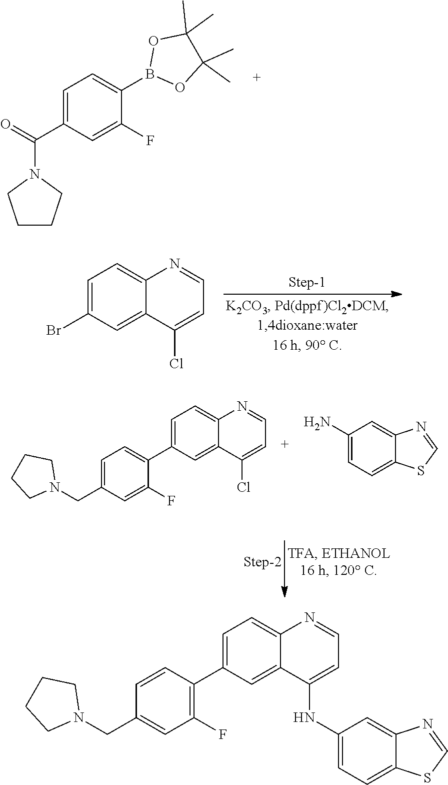 [SMILES: CC1(C)OB(c2ccc(C(=O)N3CCCC3)cc2F)OC1(C)C ; Clc1ccnc2ccc(Br)cc12 ; Fc1cc(CN2CCCC2)ccc1-c1ccc2nccc(Cl)c2c1 ; Nc1ccc2scnc2c1 ; Fc1cc(CN2CCCC2)ccc1-c1ccc2nccc(Nc3ccc4scnc4c3)c2c1]

### Step-1: Synthesis of (4-(4-chloroquinolin-6-yl)-3-fluorophenyl)(pyrrolidin-1-yl)methanone

To a stirred solution of 6-bromo-4-chloroquinoline (200 mg, 0.824 mmol, 1.0 eq) and (3-fluoro-4-(4,4,5,5-tetramethyl-1,3,2-dioxaborolan-2-yl)phenyl)(pyrrolidin-1-yl)methanone (315.8 mg, 0.989 mmol, 1.2 eq) in 1,4-dioxane:water (5 mL:1 mL), was added K₂CO₃ (227.5 mg, 1.648 mmol, 2.0 eq). Reaction mixture was purged with N2 gas for 10 minutes and was added Pd(dppf)Cl2·DCM (33.7 mg, 0.0412 mmol, 0.05 eq). After addition, reaction mixture was stirred at 90° C. for 16 h. The progress of the reaction was monitored by LCMS and TLC. After completion of the reaction, reaction mixture was diluted with water (50 mL) and was extracted with EtOAc (2×100 mL), combined organic layers were dried over Na₂SO₄, filtered and concentrated under reduced pressure to afford crude which was purified by flash chromatography (eluted in 2% MeOH:DCM) to afford (4-(4-chloroquinolin-6-yl)-3-fluorophenyl)(pyrrolidin-1-yl)methanone (250 mg, 85.6%), LCMS: 355.2 [M+1]⁺.

### Step-2: Synthesis of (4-(4-(benzo[d]thiazol-5-ylamino)quinolin-6-yl)-3-fluorophenyl)(pyrrolidin-1-yl)methanone

To a stirred solution of (4-(4-chloroquinolin-6-yl)-3-fluorophenyl)(pyrrolidin-1-yl)methanone (150 mg, 0.423 mmol, 1.0 eq) and benzo[d]thiazol-5-amine (63.5 mg, 0.423 mmol, 1.0 eq) in EtOH (3 mL) was added TFA (0.1 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (50 mL) and was extracted with DCM (2×75 mL). Combined organic layers were dried over Na₂SO₄, filtered and concentrated under reduced pressure to afford which was purified by flash chromatography (eluted in 6% MeOH:DCM) to afford (4-(4-(benzo[d]thiazol-5-ylamino)quinolin-6-yl)-3-fluorophenyl)(pyrrolidin-1-yl)methanone (16 mg, 8.1%), LCMS: 469.2 [M+1]⁺, ¹H NMR (400 MHz, DMSO-d₆) δ ppm 9.43 (s, 1H) 9.31 (s, 1H) 8.66 (s, 1H) 8.52 (d, J=5.72 Hz, 1H) 8.21 (d, J=8.27 Hz, 1H) 7.99-8.11 (m, 2H) 7.90-7.99 (m, 2H) 7.74-7.81 (m, 1H) 7.54 (t, J=6.36 Hz, 2H) 7.06 (d, J=5.72 Hz, 1H) 3.41-3.60 (m, 4H) 1.78-1.92 (m, 4H).

### Example 59. Synthesis of (4-(4-(benzo[d]thiazol-5-ylamino)quinolin-7-yl)-3-fluorophenyl)(pyrrolidin-1-yl)methanone (Compound 242)

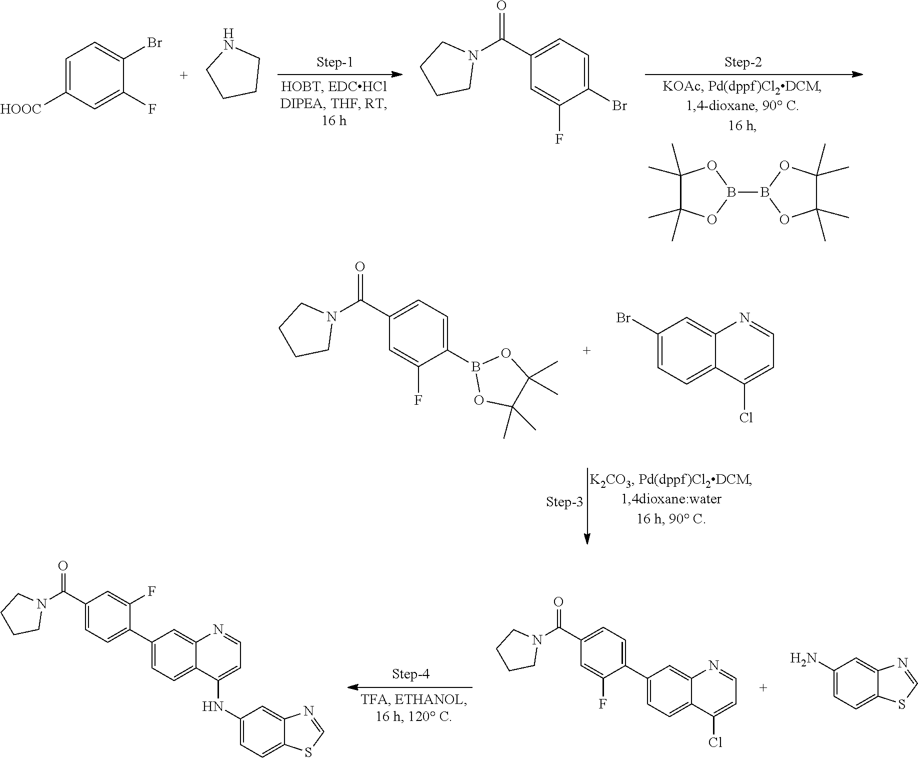

### Step-1: Synthesis of (4-bromo-3-fluorophenyl)(pyrrolidin-1-yl)methanone

To a stirred solution of 4-bromo-3-fluorobenzoic acid (200 mg, 0.913 mmol, 1.0 eq) in THF (4 mL), was added HOBT (160.4 mg, 1.186 mmol, 1.3 eq) and EDC·HCl (227.6 mg, 1.186 mmol, 1.3 eq). Reaction was stirred at RT for 1 h. Then DIPEA (0.49 mL, 2.739 mmol, 3.0 eq) and pyrrolidine (71.45 mg, 1.01 mmol, 1.1 eq) were added. Resulting reaction mass stirred at RT for 16 h. The progress of the reaction was monitored by TLC and LCMS. Upon completion of reaction, reaction mass was concentrated under reduced pressure to afford crude which was diluted with water (50 mL), and extracted by EtOAc (2×75 mL). Combined organic layers were dried over anhydrous Na₂SO₄, filtered and concentrated under reduced pressure to afford (4-bromo-3-fluorophenyl)(pyrrolidin-1-yl)methanone (240 mg, 96.38%), LCMS: 272.3[M+1]⁺.

### Step-2: Synthesis of (3-fluoro-4-(4,4,5,5-tetramethyl-1,3,2-dioxaborolan-2-yl)phenyl)(pyrrolidin-1-yl)methanone

To a stirred solution of (4-bromo-3-fluorophenyl)(pyrrolidin-1-yl)methanone (240 mg, 0.882 mmol, 1.0 eq) and Bis(pinacolato)diboron (268.7 mg, 1.058 mmol, 1.2 eq) in 1,4-Dioxane (5 mL) was added KOAc (173.2 mg, 1.764 mmol, 2.0 eq). The reaction was purged with N₂ for 10 minutes, was added Pd(dppf)Cl₂·DCM (36.01 mg, 0.044 mmol, 0.05 eq). The resulting reaction mass was again purged with N₂ for 10 minutes and after purging, reaction mixture was stirred at 100° C. for 16 h. The progress of the reaction was monitored by LCMS and TLC. After completion of the reaction, reaction mixture was diluted with water (75 mL) and was extracted with EtOAc (2×100 mL). Combined organic layers were dried over Na₂SO₄, filtered and concentrated under reduced pressure to afford (3-fluoro-4-(4,4,5,5-tetramethyl-1,3,2-dioxaborolan-2-yl)phenyl)(pyrrolidin-1-yl)methanone (250 mg, 88.65%), LCMS: 238.3[M+1]⁺.

### Step-3: Synthesis of (4-(4-chloroquinolin-7-yl)-3-fluorophenyl)(pyrrolidin-1-yl)methanone

To a stirred solution of 7-bromo-4-chloroquinoline (200 mg, 0.824 mmol, 1.0 eq) and (3-fluoro-4-(4,4,5,5-tetramethyl-1,3,2-dioxaborolan-2-yl)phenyl)(pyrrolidin-1-yl)methanone (315.8 mg, 0.989 mmol, 1.2 eq) in 1,4-dioxane:water (5 mL:1 mL) was added K₂CO₃ (227.5 mg, 1.648 mmol, 2.0 eq). Reaction mixture was purged with N2 gas for 10 minutes and was added Pd(dppf)Cl2·DCM (33.7 mg, 0.0412 mmol, 0.05 eq). Reaction mixture was stirred at 90° C. for 16 h. The progress of the reaction was monitored by LCMS and TLC. After completion of reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford (4-(4-chloroquinolin-7-yl)-3-fluorophenyl)(pyrrolidin-1-yl)methanone (119 mg, 40.75%), LCMS: 355.2 [M+1]⁺.

### Step-4: Synthesis of (4-(4-(benzo[d]thiazol-5-ylamino)quinolin-7-yl)-3-fluorophenyl)(pyrrolidin-1-yl)methanone

To a solution of (4-(4-chloroquinolin-7-yl)-3-fluorophenyl)(pyrrolidin-1-yl)methanone (100 mg, 0.281 mmol, 1.0 eq) and benzo[d]thiazol-5-amine (42.4 mg, 0.281 mmol, 1.0 eq) in EtOH (2 mL) was added TFA (0.07 mL) and the reaction mixture was stirred at 120° C. for 16 h. The progress of reaction was monitored by TLC and LCMS. After completion of reaction, reaction mixture was concentrated under reduced pressure to obtain crude which was neutralized with saturated NaHCO₃ solution (25 mL) and was extracted with DCM (2×50 mL). Combined organic layers were dried over Na2SO4, filtered and concentrated under reduced pressure to afford which was purified by flash chromatography (eluted in 6% MeOH:DCM) to afford (4-(4-(benzo[d]thiazol-5-ylamino)quinolin-7-yl)-3-fluorophenyl)(pyrrolidin-1-yl)methanone (60 mg, 45.4%), LCMS: 469.3 [M+1]⁺, ¹H NMR (400 MHz, DMSO-d₆) δ ppm 9.43 (s, 1H) 9.30 (br. s., 1H) 8.49-8.60 (m, 2H) 8.21 (d, J=8.90 Hz, 1H) 8.10 (s, 1H) 8.05 (s, 1H) 7.77-7.90 (m, 2H) 7.58 (br. s., 1H) 7.49-7.56 (m, 2H) 7.05 (d, J=5.09 Hz, 1H) 3.50 (d, J=6.99 Hz, 4H) 1.81-1.96 (m, 4H).

### Example 60. Synthesis of (4-(4-(benzo[d]thiazol-5-ylamino)quinolin-6-yl)-3-fluorophenyl)(morpholino)methanone (Compound 243)

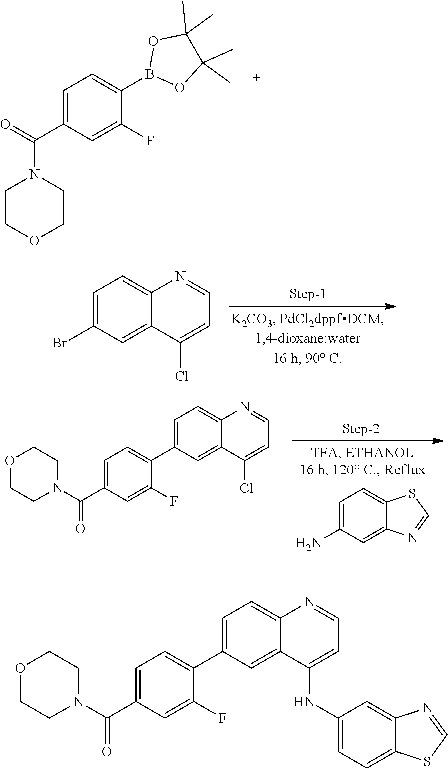

### Step-1: Synthesis of (4-(4-chloroquinolin-6-yl)-3-fluorophenyl)(morpholino)methanone

To a stirred solution of 6-bromo-4-chloroquinoline (175 mg, 0.721 mmol, 1.0 eq) and (3-fluoro-4-(4,4,5,5-tetramethyl-1,3,2-dioxaborolan-2-yl)phenyl)(morpholino)methanone (290.3 mg, 0.866 mmol, 1.2 eq) in 1,4-dioxane:water (4 mL:0.5 mL), was added K₂CO₃ (198.9 mg, 1.442 mmol, 2.0 eq). Reaction mixture was purged with N2 gas for 10 minutes and was added Pd(dppf)Cl2·DCM (29.5 mg, 0.036 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×100 mL), combined organic layers were dried over Na₂SO₄, filtered and concentrated under reduced pressure to afford crude which was purified by flash chromatography (eluted in 2% MeOH:DCM) to afford (4-(4-chloroquinolin-6-yl)-3-fluorophenyl)(morpholino)methanone (170 mg, 63.6%), LCMS: 371.3 [M+1]⁺.

### Step-2: Synthesis of (4-(4-(benzo[d]thiazol-5-ylamino)quinolin-6-yl)-3-fluorophenyl)(morpholino)methanone

To a solution of (4-(4-chloroquinolin-6-yl)-3-fluorophenyl)(morpholino)methanone (170 mg, 0.458 mmol, 1.0 eq) and benzo[d]thiazol-5-amine (68.86 mg, 0.458 mmol, 1.0 eq) in EtOH (4 mL) was added TFA (0.15 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (50 mL) and was extracted with DCM (2×100 mL). Combined organic layers were dried over Na₂SO₄, filtered and concentrated under reduced pressure to afford which was purified by flash chromatography (eluted in 6% MeOH:DCM) to afford (4-(4-(benzo[d]thiazol-5-ylamino)quinolin-6-yl)-3-fluorophenyl)(morpholino)methanone (46 mg, 20.7%). ANALYTICAL DATA: LCMS: 485.3 [M+1]⁺, ¹H NMR (400 MHz, DMSO-d₆) δ ppm 9.44 (s, 1H) 9.37 (br. s., 1H) 8.66 (s, 1H) 8.52 (d, J=5.09 Hz, 1H) 8.21 (d, J=8.27 Hz, 1H) 7.95-8.04 (m, 2H) 7.94 (s, 1H) 7.81-7.88 (m, 1H) 7.56 (d, J=8.27 Hz, 2H) 7.48 (d, J=10.81 Hz, 1H) 7.05 (d, J=5.09 Hz, 1H) 3.65 (br. s., 4H) 3.52 (m, 4H).

### Example 61. Synthesis of (4-(4-(benzo[d]thiazol-5-ylamino)quinolin-7-yl)-3-fluorophenyl)(morpholino)methanone (Compound 244)

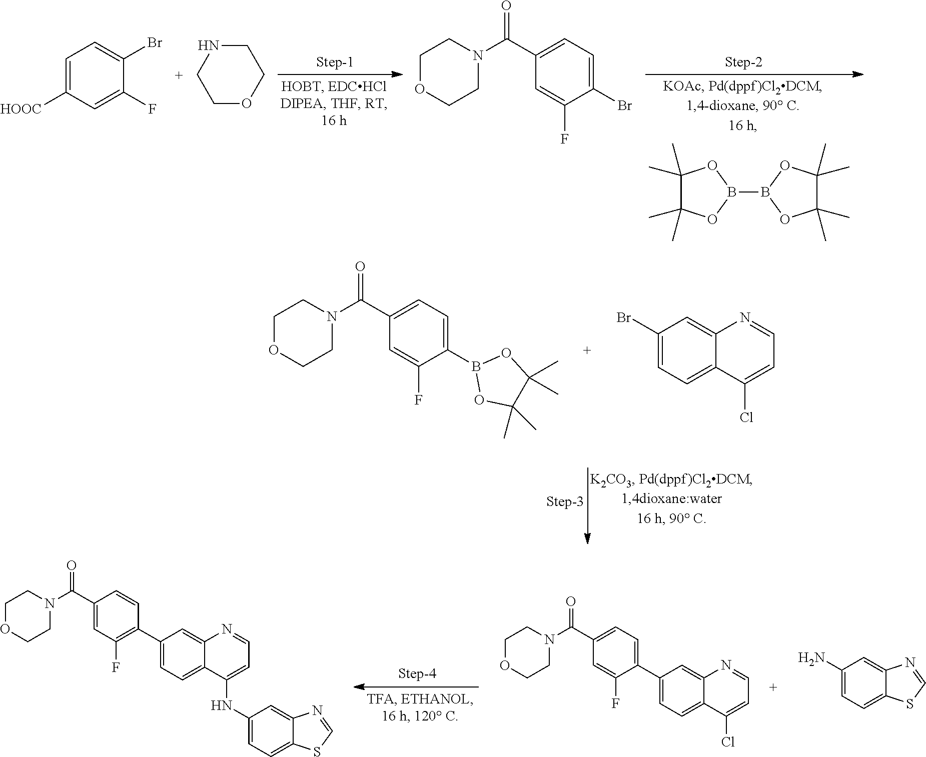

### Step-1: Synthesis of (4-bromo-3-fluorophenyl)(morpholino)methanone

To a stirred solution of 4-bromo-3-fluorobenzoic acid (200 mg, 0.913 mmol, 1.0 eq) in THF (4 mL), was added HOBT (160.4 mg, 1.186 mmol, 1.3 eq) and EDC·HCl (227.6 mg, 1.186 mmol, 1.3 eq) at room temperature. After addition, reaction mixture was stirred at RT for 1 h and was added DIPEA (0.49 mL, 2.739 mmol, 3.0 eq) and morpholine (71.45 mg, 1.01 mmol, 1.1 eq). Resulting reaction mass was stirred at RT for 16 h. The progress of the reaction was monitored by TLC and LCMS. After completion of the reaction, reaction mixture was concentrated under reduced pressure to afford crude which was diluted with water (50 mL) and was extracted by EtOAc (2×75 mL). Combined organic layers were dried over anhydrous Na₂SO₄, filtered and concentrated under reduced pressure to afford (4-bromo-3-fluorophenyl)(morpholino)methanone (260 mg, 99.2%), LCMS: 288.2[M+1]⁺.

### Step-2: Synthesis of (3-fluoro-4-(4,4,5,5-tetramethyl-1,3,2-dioxaborolan-2-yl)phenyl)(morpholino)methanone

To a stirred solution of (4-bromo-3-fluorophenyl)(morpholino)methanone (260 mg, 0.902 mmol, 1.0 eq) and Bis(pinacolato)diboron (274.9 mg, 1.08 mmol, 1.2 eq) in 1,4-Dioxane (4 mL), was added KOAc (265.6 mg, 2.706 mmol, 3.0 eq). The reaction was purged with N₂ for 10 minutes and was added Pd(dppf)Cl₂·DCM (36.8 mg, 0.0451 mmol, 0.05 eq). The resulting reaction mass was again purged with N₂ for 10 minutes and stirred at 100° C. for 16 h. The progress of the reaction was monitored by LCMS and TLC. After completion of reaction, reaction mixture was diluted with water (75 mL) and was extracted with EtOAc (2×100 mL). Combined organic layers were dried over Na₂SO₄, filtered and concentrated under reduced pressure to afford (3-fluoro-4-(4,4,5,5-tetramethyl-1,3,2-dioxaborolan-2-yl)phenyl)(morpholino)methanone (290 mg, 95.7%), LCMS: 254.3[M+1]⁺(Boronic-acid fragment).

### Step-3: Synthesis of (4-(4-chloroquinolin-7-yl)-3-fluorophenyl)(morpholino)methanone

To a stirred solution of 7-bromo-4-chloroquinoline (170 mg, 0.701 mmol, 1.0 eq) and (3-fluoro-4-(4,4,5,5-tetramethyl-1,3,2-dioxaborolan-2-yl)phenyl)(morpholino)methanone (281.96 mg, 0.841 mmol, 1.2 eq) in 1,4-dioxane:water (4 mL:0.5 mL), was added K₂CO₃ (193.4 mg, 1.402 mmol, 2.0 eq). Reaction mixture was purged with N2 gas for 10 minutes and was added Pd(dppf)Cl2·DCM (28.62 mg, 0.0350 mmol, 0.05 eq). Reaction mixture was stirred at 90° C. for 16 h. The progress of the reaction was monitored by LCMS and TLC. After completion of reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford (4-(4-chloroquinolin-7-yl)-3-fluorophenyl)(morpholino)methanone (155 mg, 58.05%), LCMS: 371.3 [M+1]⁺.

### Step-4: Synthesis of (4-(4-(benzo[d]thiazol-5-ylamino)quinolin-7-yl)-3-fluorophenyl)(morpholino)methanone

To a solution of (4-(4-chloroquinolin-7-yl)-3-fluorophenyl)(morpholino)methanone (155 mg, 0.418 mmol, 1.0 eq) and benzo[d]thiazol-5-amine (63.1 mg, 0.418 mmol, 1.0 eq) in EtOH (4 mL) was added TFA (0.12 mL) and the reaction mixture was stirred at 120° C. for 16 h. The progress of the reaction was monitored by TLC and LCMS. After completion of reaction, reaction mixture was concentrated under reduced pressure to obtain crude which was neutralized with saturated NaHCO₃ solution (25 mL) and was extracted with DCM (2×75 mL). Combined organic layers were dried over Na2SO4, filtered and concentrated under reduced pressure to afford which was purified by flash chromatography (eluted in 6% MeOH:DCM) to afford (4-(4-(benzo[d]thiazol-5-ylamino)quinolin-7-yl)-3-fluorophenyl)(morpholino)methanone (57 mg, 28.2%). ANALYTICAL DATA: LCMS: 485.3 [M+1]⁺, ¹H NMR (400 MHz, DMSO-d₆) δ ppm 9.43 (s, 1H) 9.30 (br. s., 1H) 8.47-8.60 (m, 2H) 8.21 (d, J=8.27 Hz, 1H) 8.09 (s, 1H) 8.04-8.06 (m, 1H) 7.79-7.88 (m, 2H) 7.56 (d, J=8.90 Hz, 1H) 7.38-7.47 (m, 2H) 7.05 (d, J=5.09 Hz, 1H) 3.64 (br. s., 4H) 3.72 (br. s., 4H).

### Example 62. Synthesis of 4-(4-(benzo[d]thiazol-5-ylamino)quinolin-6-yl)-3-fluoro-N,N-dimethylbenzamide (Compound 245)

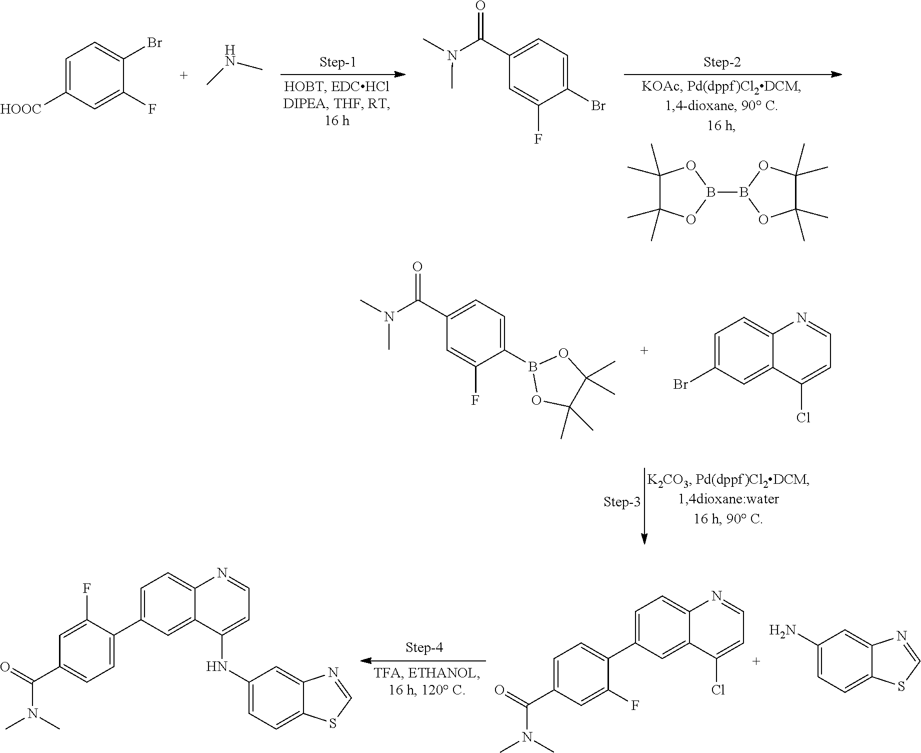

### Step-1: Synthesis of 4-bromo-3-fluoro-N,N-dimethylbenzamide

To a stirred solution of 4-bromo-3-fluorobenzoic acid (400 mg, 1.83 mmol, 1.0 eq) in THF (6 mL), was added HOBT (321 mg, 2.37 mmol, 1.3 eq) and EDC·HCl (456 mg, 2.37 mmol, 1.3 eq). Reaction mixture was stirred at RT for 1 h. Then DIPEA (711 mg, 5.5 mmol, 3.0 eq) and Dimethylamine (10% in THF sol, 826 mg, 18.3 mmol, and 10 eq) were added. Resulting reaction mass was stirred at RT for 16 h. The progress of the reaction was monitored by TLC and LCMS. After completion of the reaction, reaction mixture was concentrated under reduced pressure to afford crude which was diluted with water (50 mL), and product was extracted by EtOAc (2×100 mL), Combined organic layers were dried over anhydrous Na₂SO₄, filtered and concentrated under reduced pressure to afford 4-bromo-3-fluoro-N,N-dimethylbenzamide (380 mg, 84.82%), LCMS: 246 [M+1]⁺.

### Step-2: Synthesis of 3-fluoro-N,N-dimethyl-4-(4,4,5,5-tetramethyl-1,3,2-dioxaborolan-2-yl)benzamide

To a stirred solution of 4-bromo-3-fluoro-N,N-dimethylbenzamide (190 mg, 0.775 mmol, 1.0 eq) and Bis(pinacolato)diboron (217 mg, 0.853 mmol, 1.1 eq) in 1,4-Dioxane (5 mL), was added KOAc (228.2 mg, 2.325 mmol, 3.0 eq). The reaction mixture was purged with N₂ for 10 minutes and was added Pd(dppf)Cl₂·DCM (31.6 mg, 0.038 mmol, 0.05 eq). The resulting reaction mass was again purged with N₂ for 10 minutes and stirred at 100° C. for 16 h. The progress of the reaction was monitored by LCMS and TLC. After completion of the reaction, reaction mixture was diluted with water (75 mL) and was extracted with EtOAc (2×100 mL), combined organic layers were dried over Na₂SO₄, filtered and concentrated under reduced pressure to afford 3-fluoro-N,N-dimethyl-4-(4,4,5,5-tetramethyl-1,3,2-dioxaborolan-2-yl)benzamide (210 mg, 92.5%), LCMS: 294[M+1]⁺.

### Step-3: Synthesis of 4-(4-chloroquinolin-6-yl)-3-fluoro-N,N-dimethylbenzamide

To a stirred solution of 6-bromo-4-chloroquinoline (210 mg, 0.863 mmol, 1.0 eq) and 3-fluoro-N,N-dimethyl-4-(4,4,5,5-tetramethyl-1,3,2-dioxaborolan-2-yl)benzamide (304 mg, 1.039 mmol, 1.2 eq) in 1,4-dioxane:water (5 mL:1 mL), was added K₂CO₃ (180 mg, 1.298 mmol, 1.5 eq). Reaction mixture was purged with N2 gas for 10 minutes and was added Pd(dppf)Cl2·DCM (35.5 mg, 0.0432 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford 4-(4-chloroquinolin-6-yl)-3-fluoro-N,N-dimethylbenzamide (140 mg, 49.46%), LCMS: 329[M+1]⁺.

### Step-4: Synthesis of 4-(4-(benzo[d]thiazol-5-ylamino)quinolin-6-yl)-3-fluoro-N,N-dimethylbenzamide

To a stirred solution of 4-(4-chloroquinolin-6-yl)-3-fluoro-N,N-dimethylbenzamide (140 mg, 0.426 mmol, 1.0 eq) and benzo[d]thiazol-5-amine (64.02 mg, 0.426 mmol, 1.0 eq) in EtOH (4 mL) was added TFA (0.1 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (25 mL) and was extracted with DCM (2×50 mL), combined organic layers were dried over Na₂SO₄, filtered and concentrated under reduced pressure to afford crude which was purified by flash chromatography, eluted in 6% MeOH:DCM to afford 4-(4-(benzo[d]thiazol-5-ylamino)quinolin-6-yl)-3-fluoro-N,N-dimethylbenzamide (50 mg, 26.5%), LCMS: 443.3 [M+1]⁺, ¹H NMR (400 MHz, DMSO-d₆) δ ppm 9.43 (s, 1H) 9.30 (s, 1H) 8.65 (s, 1H) 8.52 (d, J=5.09 Hz, 1H) 8.21 (d, J=8.90 Hz, 1H) 8.04 (s, 1H) 7.99 (d, J=8.27 Hz, 1H) 7.92 (d, J=8.90 Hz, 1H) 7.80 (t, J=7.95 Hz, 1H) 7.56 (d, J=8.90 Hz, 1H) 7.40-7.48 (m, 2H) 7.06 (d, J=5.09 Hz, 1H) 2.98 (s, 3H) 3.02 (s, 3H).

### Example 63. Synthesis of 4-(4-(benzo[d]thiazol-5-ylamino)quinolin-7-yl)-3-fluoro-N,N-dimethylbenzamide (Compound 246)

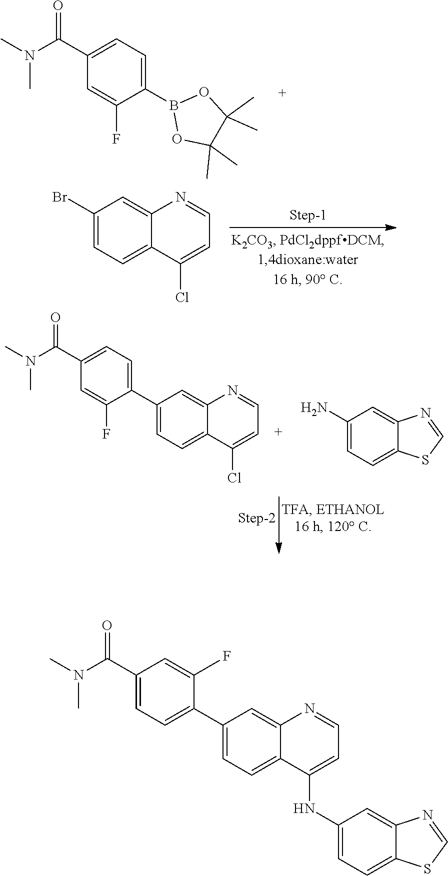

### Step-1: Synthesis of 4-(4-chloroquinolin-7-yl)-3-fluoro-N,N-dimethylbenzamide

To a stirred solution of 7-bromo-4-chloroquinoline (288 mg, 1.187 mmol, 1.0 eq) and 3-fluoro-N,N-dimethyl-4-(4,4,5,5-tetramethyl-1,3,2-dioxaborolan-2-yl)benzamide (417.7 mg, 1.424 mmol, 1.2 eq) in 1,4-dioxane:water (4 mL:1 mL), was added K₂CO₃ (245.7 mg, 1.780 mmol, 1.5 eq). Reaction mixture was purged with N2 gas for 10 minutes and was added Pd(dppf)Cl2·DCM (48.5 mg, 0.0593 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford 4-(4-chloroquinolin-7-yl)-3-fluoro-N,N-dimethylbenzamide (140 mg, 35.89%). LCMS: 329 [M+1]⁺.

### Step-2: Synthesis of 4-(4-(benzo[d]thiazol-5-ylamino)quinolin-7-yl)-3-fluoro-N,N-dimethylbenzamide

To a solution of 4-(4-chloroquinolin-7-yl)-3-fluoro-N,N-dimethylbenzamide (140 mg, 0.426 mmol, 1.0 eq) and benzo[d]thiazol-5-amine (64.02 mg, 0.426 mmol, 1.0 eq) in EtOH (4 mL) was added TFA (0.1 mL) and the reaction mixture was stirred at 120° C. for 16 h. The progress of the reaction was monitored by TLC and LCMS. After completion of reaction, reaction mixture was concentrated under reduced pressure to obtain crude which was neutralized with saturated NaHCO₃ solution (25 mL) and desired product was extracted with DCM (2×50 mL), combined organic layers were dried over Na2SO4, filtered and concentrated under reduced pressure to afford crude product which was purified by flash chromatography eluted in 6% MeOH:DCM to afford 4-(4-(benzo[d]thiazol-5-ylamino)quinolin-7-yl)-3-fluoro-N,N-dimethylbenzamide (12.8 mg, 6.8%), LCMS: 443.3 [M+1]⁺, ¹H NMR (400 MHz, DMSO-d₆) Q ppm 9.43 (s, 1H) 9.27 (s, 1H) 8.54 (d, J=5.72 Hz, 2H) 8.21 (d, J=8.27 Hz, 1H) 8.10 (s, 1H) 8.05 (s, 1H) 7.75-7.84 (m, 2H) 7.57 (d, J=8.90 Hz, 1H) 7.37-7.50 (m, 2H) 7.05 (d, J=5.09 Hz, 1H) 3.01 (d, J=9.54 Hz, 6H).

### Example 64. Synthesis of (4-(4-(benzo[d]thiazol-5-ylamino)quinolin-6-yl)-3-fluorophenyl)(2-methylpiperidin-1-yl)methanone (Compound 247)

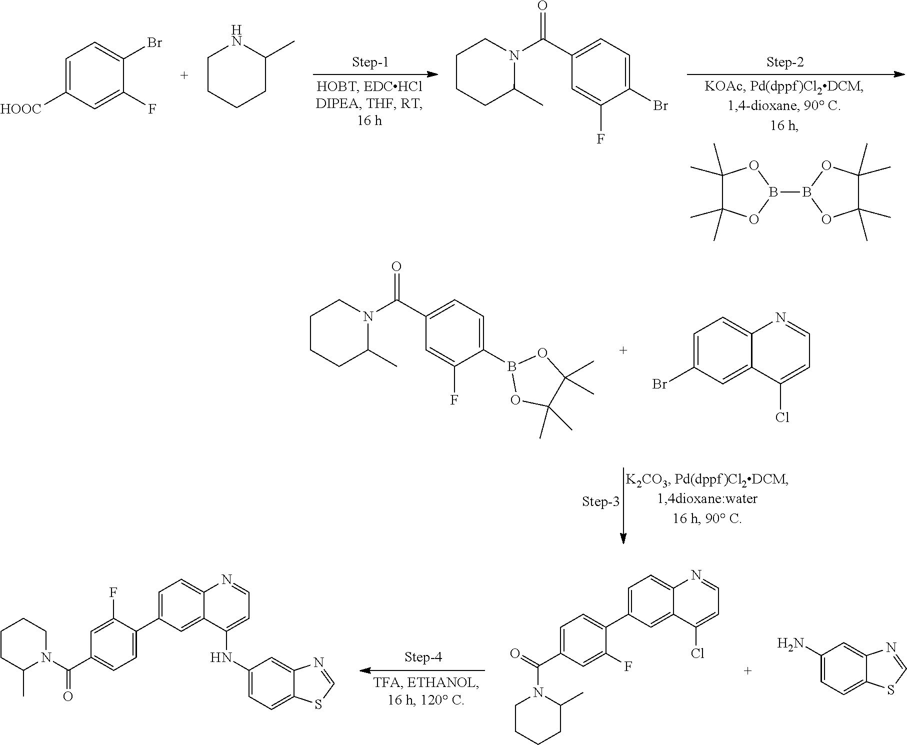

### Step-1: Synthesis of (4-bromo-3-fluorophenyl)(2-methylpiperidin-1-yl)methanone

To a stirred solution of 4-bromo-3-fluorobenzoic acid (200 mg, 0.917 mmol, 1.0 eq) in THF (5 mL), was added HOBT (160.4 mg, 1.192 mmol, 1.3 eq) and EDC·HCl (176.6 mg, 1.12 mmol, 1.3 eq). Reaction was stirred at RT for 1 h, then was added DIPEA (355.5 mg, 2.75 mmol, 3.0 eq) and 2-methylpiperidine (109 mg, 1.10 mmol, 1.2 eq). Resulting reaction mass was stirred at RT for 16 h. The progress of the reaction was monitored by TLC and LCMS. After completion of the reaction, reaction mixture was concentrated under reduced pressure to afford crude which was diluted with water (50 mL), and product was extracted by EtOAc (2×75 mL), combined organic layers were dried over anhydrous Na₂SO₄, filtered and concentrated under reduced pressure to afford (4-bromo-3-fluorophenyl)(2-methylpiperidin-1-yl)methanone (250 mg, 91.5%), LCMS: 300[M+1]⁺.

### Step-2: Synthesis of (3-fluoro-4-(4,4,5,5-tetramethyl-1,3,2-dioxaborolan-2-yl)phenyl)(2-methylpiperidin-1-yl)methanone

To a stirred solution of (4-bromo-3-fluorophenyl)(2-methylpiperidin-1-yl)methanone (280 mg, 0.936 mmol, 1.0 eq) and Bis(pinacolato)diboron (261.6 mg, 1.03 mmol, 1.1 eq) in 1,4-Dioxane (5 mL), was added KOAc (275 mg, 2.808 mmol, 3.0 eq). The reaction was purged with N₂ for 10 minutes. Further, was added Pd(dppf)Cl₂·DCM (38 mg, 0.046 mmol, 0.05 eq). The resulting reaction mass was again purged with N₂ for 10 minutes and stirred at 100° C. for 16 h. The progress of the reaction was monitored by LCMS and TLC. After completion of reaction, reaction mixture was diluted with water (75 mL) and was extracted with EtOAc (2×100 mL), combined organic layers were dried over Na₂SO₄, filtered and concentrated under reduced pressure to afford (3-fluoro-4-(4,4,5,5-tetramethyl-1,3,2-dioxaborolan-2-yl)phenyl)(2-methylpiperidin-1-yl)methanone (160 mg, 49.2%), LCMS: 348[M+1]⁺.

### Step-3: Synthesis of (4-(4-chloroquinolin-6-yl)-3-fluorophenyl)(2-methylpiperidin-1-yl)methanone

To a stirred solution of 6-bromo-4-chloroquinoline (160 mg, 0.659 mmol, 1.0 eq) and (3-fluoro-4-(4,4,5,5-tetramethyl-1,3,2-dioxaborolan-2-yl)phenyl)(2-methylpiperidin-1-yl)methanone (275 mg, 0.790 mmol, 1.2 eq) in 1,4-dioxane:water (4 mL:1 mL), was added K₂CO₃ (136.5 mg, 0.988 mmol, 1.5 eq). Reaction mixture was purged with N2 gas for 10 minutes and was added Pd(dppf)Cl2·DCM (26.9 mg, 0.032 mmol, 0.05 eq). Reaction mixture was stirred at 90° C. for 16 h. The progress of the reaction was monitored by LCMS and TLC. After completion of the reaction, reaction mixture was diluted with water (50 mL) and was extracted with EtOAc (2×75 mL), combined organic layers were dried over Na₂SO₄, filtered and concentrated under reduced pressure to afford crude which was purified by flash chromatography (eluted in 2% MeOH:DCM) to afford (4-(4-chloroquinolin-6-yl)-3-fluorophenyl)(2-methylpiperidin-1-yl)methanone (140 mg, 55.5%), LCMS: 383[M+1]⁺.

### Step-4: Synthesis of (4-(4-(benzo[d]thiazol-5-ylamino)quinolin-6-yl)-3-fluorophenyl)(2-methylpiperidin-1-yl)methanone

To a solution of (4-(4-chloroquinolin-6-yl)-3-fluorophenyl)(2-methylpiperidin-1-yl)methanone (140 mg, 0.366 mmol, 1.0 eq) and benzo[d]thiazol-5-amine (54.9 mg, 0.366 mmol, 1.0 eq) in EtOH (4 mL) was added TFA (0.1 mL) and the reaction mixture was heated at 120° C. for 16 h. The progress of the reaction was monitored by TLC and LCMS. After completion of the reaction, reaction mixture was concentrated under reduced pressure to obtain crude which was neutralized with saturated NaHCO₃ solution (25 mL) and was extracted with DCM (2×50 mL), combined organic layers were dried over Na₂SO₄, filtered and concentrated under reduced pressure to afford which was purified by flash chromatography (eluted in 6% MeOH:DCM) to afford (4-(4-(benzo[d]thiazol-5-ylamino)quinolin-6-yl)-3-fluorophenyl)(2-methylpiperidin-1-yl)methanone (22 mg, 12.15%), LCMS: 497.3 [M+1]⁺, ¹H NMR (400 MHz, DMSO-d₆) δ ppm 9.43 (s, 1H) 9.35 (br. s., 1H) 8.66 (s, 1H) 8.52 (d, J=5.72 Hz, 1H) 8.16-8.27 (m, 1H) 8.04 (d, J=1.27 Hz, 1H) 7.94-8.01 (m, 1H) 7.92 (br. s., 1H) 7.79 (t, J=7.95 Hz, 1H) 7.56 (dd, J=8.90, 1.91 Hz, 1H) 7.32-7.46 (m, 2H) 7.05 (d, J=5.09 Hz, 1H) 3.6 (m, 2H) 2.5 (m, 1H) 1.55-1.72 (m, 6H) 1.35 (s, 3H).

### Example 65. Synthesis of (4-(4-(benzo[d]thiazol-5-ylamino)quinolin-7-yl)-3-fluorophenyl)(2-methylpiperidin-1-yl)methanone (Compound 248)

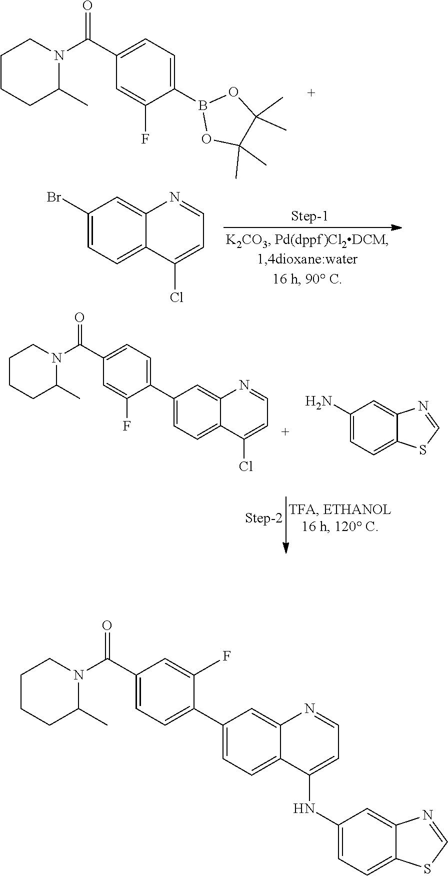 [SMILES: CC1CCCCN1C(=O)c1ccc(B2OC(C)(C)C(C)(C)O2)c(F)c1 ; Clc1ccnc2cc(Br)ccc12 ; CC1CCCCN1C(=O)c1ccc(-c2ccc3c(Cl)ccnc3c2)c(F)c1 ; Nc1ccc2scnc2c1 ; CC1CCCCN1C(=O)c1ccc(-c2ccc3c(Nc4ccc5scnc5c4)ccnc3c2)c(F)c1]

### Step-1: Synthesis of (4-(4-chloroquinolin-7-yl)-3-fluorophenyl)(2-methylpiperidin-1-yl)methanone

To a stirred solution of 7-bromo-4-chloroquinoline (160 mg, 0.659 mmol, 1.0 eq) and (3-fluoro-4-(4,4,5,5-tetramethyl-1,3,2-dioxaborolan-2-yl)phenyl)(2-methylpiperidin-1-yl)methanone (275 mg, 0.791 mmol, 1.2 eq) in 1,4-dioxane:water (10 mL:2 mL), was added K₂CO₃ (136.5 mg, 0.989 mmol, 1.5 eq). Reaction mixture was purged with N2 gas for 10 minutes and was added Pd(dppf)Cl2·DCM (27 mg, 0.032 mmol, 0.05 eq). Reaction mixture was stirred at 90° C. for 16 h. The progress of the reaction was monitored by LCMS and TLC. After completion of the reaction, reaction mixture was diluted with water (50 mL) and extracted with EtOAc (2×75 mL), combined organic layers were dried over Na₂SO₄, filtered and concentrated under reduced pressure to afford crude which was purified by flash chromatography (eluted in 2% MeOH:DCM) to afford (4-(4-chloroquinolin-7-yl)-3-fluorophenyl)(2-methylpiperidin-1-yl)methanone (100 mg, 39.68%), LCMS: 383[M+1]⁺.

### Step-2: Synthesis of (4-(4-(benzo[d]thiazol-5-ylamino)quinolin-7-yl)-3-fluorophenyl)(2-methylpiperidin-1-yl)methanone

To a stirred solution of (4-(4-chloroquinolin-7-yl)-3-fluorophenyl)(2-methylpiperidin-1-yl)methanone (140 mg, 0.336 mmol, 1.0 eq) and benzo[d]thiazol-5-amine (55 mg, 0.336 mmol, 1.0 eq) in EtOH (4 mL) was added TFA (0.1 mL) and the reaction mixture was stirred at 120° C. for 16 h. The progress of the reaction was monitored by TLC and LCMS. After completion of the reaction, reaction mixture was concentrated under reduced pressure to obtain crude which was neutralized with saturated NaHCO₃ solution (25 mL) and was extracted with DCM (2×50 mL), combined organic layers were dried over Na2SO4, filtered and concentrated under reduced pressure to afford which was purified by flash chromatography (eluted in 6% MeOH:DCM) to afford (4-(4-(benzo[d]thiazol-5-ylamino)quinolin-7-yl)-3-fluorophenyl)(2-methylpiperidin-1-yl)methanone (22 mg, 12.14%), LCMS: 497.3 [M+1]⁺, ¹H NMR (400 MHz, DMSO-d₆) δ ppm 9.44 (s, 1H) 9.32 (br. s., 1H) 8.49-8.58 (m, 2H) 8.21 (d, J=8.27 Hz, 1H) 8.10 (s, 1H) 8.05 (d, J=1.27 Hz, 1H) 7.76-7.83 (m, 2H) 7.52-7.61 (m, 1H) 7.40 (d, J=11.44 Hz, 1H) 7.35 (d, J=7.63 Hz, 1H) 7.05 (d, J=5.09 Hz, 1H) 3.5 (m, 2H) 2.8 (m, 1H) 1.67 (br. s., 6H) 1.55 (s, 3H).

### Example 66: Synthesis of N-(7-(4-(piperidin-4-yl)phenyl)quinolin-4-yl)benzo[d]thiazol-5-amine (Compound 253)

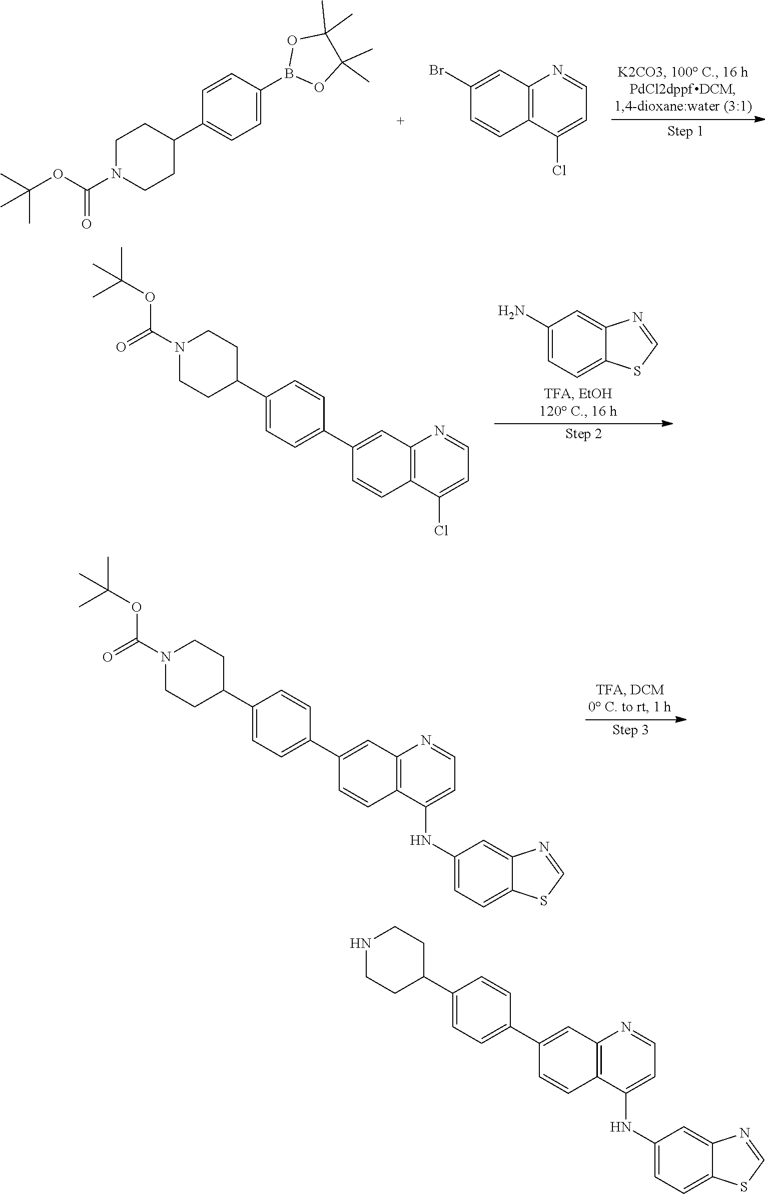

### Step-1: Synthesis of tert-butyl 4-(4-(4-chloroquinolin-7-yl)phenyl)piperidine-1-carboxylate

To a solution tert-butyl 4-(4-(4,4,5,5-tetramethyl-1,3,2-dioxaborolan-2-yl)phenyl)piperidine-1-carboxylate (100 mg, 0.258 mmol, 1.0 equiv) in 1,4-dioxane:water (3.5 mL:1.5 mL) was added K₂CO₃ (71 mg, 0.516 mmol, 2.0 equiv) and 7-bromo-4-chloroquinoline (62 mg, 0.258 mmol, 1.0 equiv). Reaction mixture was purged with nitrogen for 5 min, was added PdCl₂dppf·DCM (10 mg, 0.012 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography to afford tert-butyl 4-(4-(4-chloroquinolin-7-yl)phenyl)piperidine-1-carboxylate (105 mg, 96.3%), LCMS: 423.3[M+1]⁺.

### Step-2: Synthesis of tert-butyl 4-(4-(4-(benzo[d]thiazol-5-ylamino)quinolin-7-yl)phenyl)piperidine-1-carboxylate

To a solution of tert-butyl 4-(4-(4-chloroquinolin-7-yl)phenyl)piperidine-1-carboxylate (105 mg, 0.248 mmol, 1.0 equiv) in ethanol (5 mL) was added benzo[d]thiazol-5-amine (55 mg, 0.372 mmol, 1.5 equiv) and TFA (0.1 mL). The reaction mixture was heated at 120° C. for 16 h. After completion of reaction, reaction mixture was concentrated under vacuum to obtain crude which was neutralized by saturated sodium bicarbonate solution (30 mL), was extracted with ethyl acetate (2×60 mL), dried over anhydrous sodium sulphate, filtered and concentrated under vacuum to afford crude which was purified by flash chromatography to afford tert-butyl 4-(4-(4-(benzo[d]thiazol-5-ylamino)quinolin-7-yl)phenyl)piperidine-1-carboxylate (80 mg, 60.1%), LCMS: 537.2[M+1]⁺.

### Step-3: Synthesis of N-(7-(4-(piperidin-4-yl)phenyl)quinolin-4-yl)benzo[d]thiazol-5-amine

To a solution of tert-butyl 4-(4-(4-(benzo[d]thiazol-5-ylamino)quinolin-7-yl)phenyl)piperidine-1-carboxylate (80 mg, 0.149 mmol, 1.0 equiv) in dichloromethane (6 mL) was added TFA (0.4 mL) at 0° C. The reaction mixture was stirred at RT for 1 h. After completion of reaction, reaction mixture was concentrated under vacuum and co-distilled with ether several times to remove TFA and to afford N-(7-(4-(piperidin-4-yl)phenyl)quinolin-4-yl)benzo[d]thiazol-5-amine (37 mg, 56.92%), LCMS: 437.2[M+1]⁺. ¹H NMR (400 MHz, DMSO-d₆) δ 1.79-1.89 (m, 2H) 2.01 (d, J=12.08 Hz, 2H) 2.92-3.10 (m, 3H) 3.43 (d, J=11.44 Hz, 3H) 6.90 (d, J=6.99 Hz, 1H) 7.47 (m, J=8.27 Hz, 2H) 7.61-7.65 (m, 1H) 7.88 (m, J=8.27 Hz, 2H) 8.17-8.23 (m, 3H) 8.40 (d, J=8.27 Hz, 2H) 8.56 (d, J=6.99 Hz, 1H) 8.80 (d, J=8.27 Hz, 1H) 9.54 (s, 1H).

### Example 67: Synthesis of 4-(4-(4-(benzo[d]thiazol-5-ylamino)quinolin-6-yl)-3-fluorobenzoyl)-1-methylpiperazin-2-one (Compound 273)

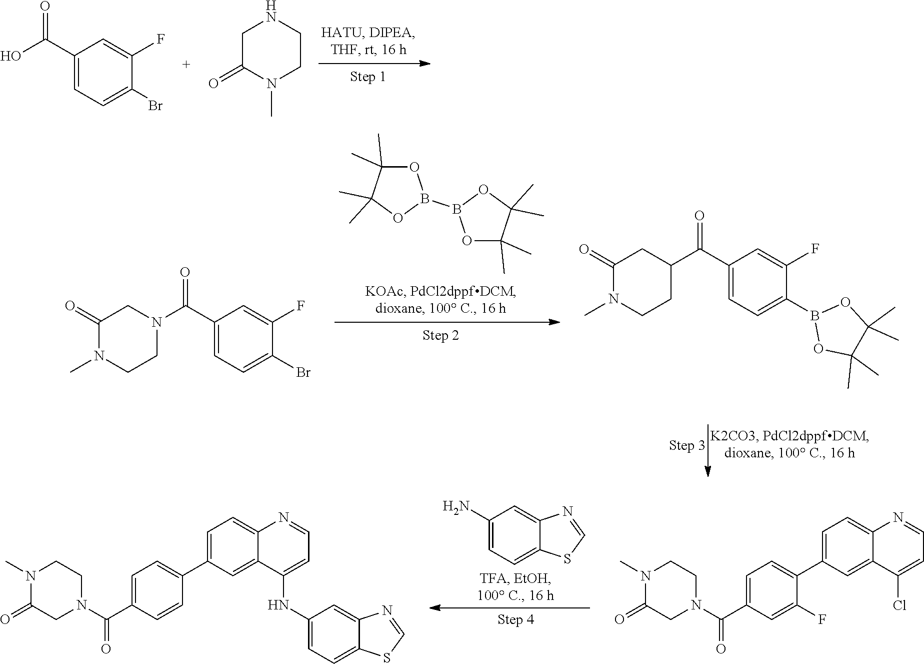

### Step-1: Synthesis of 4-(4-bromo-3-fluorobenzoyl)-1-methylpiperazin-2-one

To a solution of 4-bromo-3-fluorobenzoic acid (200 mg, 0.913 mmol, 1 equiv) in THF (5 mL) was added EDC·HCl (227 mg, 1.18 mmol, 1.3 equiv), and HOBT (160 mg, 1.18 mmol, 1.3 equiv) and stirred at RT for 1 h. Then DIPEA (0.5 mL, 2.73 mmol, 3 equiv) was added followed by the addition of 1-methylpiperazin-2-one (125 mg, 1.09 mmol, 1.2 equiv). The reaction mixture was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under vacuum to afford crude which was purified by flash chromatography to afford 4-(4-bromo-3-fluorobenzoyl)-1-methylpiperazin-2-one (280 mg, 97.5%), LCMS: 317.1[M+1]⁺.

### Step-2: Synthesis of 4-(3-fluoro-4-(4,4,5,5-tetramethyl-1,3,2-dioxaborolan-2-yl)benzoyl)-1-methylpiperazin-2-one

To a solution 4-(4-bromo-3-fluorobenzoyl)-1-methylpiperazin-2-one (280 mg, 0.888 mmol, 1 equiv) in 1,4-dioxane (5 mL) was added KOAc (261 mg, 2.66 mmol, 3 equiv) and 4,4,4′,4′,5,5,5′,5′-octamethyl-2,2′-bi(1,3,2-dioxaborolane) (270 mg, 1.066 mmol, 1.2 equiv). Reaction mixture was purged with nitrogen for 5 min, was added PdCl₂dppf·DCM (36 mg, 0.044 mmol, 0.05 equiv). The reaction mixture was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under vacuum to afford crude which was purified by flash chromatography to afford 4-(3-fluoro-4-(4,4,5,5-tetramethyl-1,3,2-dioxaborolan-2-yl)benzoyl)-1-methylpiperazin-2-one (315 mg, 98%), LCMS: 281.2[M+1]⁺.[Boronic acid fragment].

### Step-3: Synthesis of 4-(4-(4-chloroquinolin-6-yl)-3-fluorobenzoyl)-1-methylpiperazin-2-one

To a solution of 4-(3-fluoro-4-(4,4,5,5-tetramethyl-1,3,2-dioxaborolan-2-yl)benzoyl)-1-methylpiperazin-2-one (100 mg, 0.276 mmol, 1 equiv) in 1,4-dioxane:water (3.5 mL:1.5 mL) was added K₂CO₃ (76 mg, 0.552 mmol, 2 equiv) and 6-bromo-4-chloroquinoline (66 mg, 0.276 mmol, 1 equiv). Reaction mixture was purged with nitrogen for 5 min, was added PdCl₂dppf·DCM (11 mg, 0.013 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography to afford 4-(4-(4-chloroquinolin-6-yl)-3-fluorobenzoyl)-1-methylpiperazin-2-one (105 mg, 96.3%), LCMS: 398.1[M+1]⁺.

### Step-4: Synthesis of 4-(4-(4-(benzo[d]thiazol-5-ylamino)quinolin-6-yl)-3-fluorobenzoyl)-1-methylpiperazin-2-one

To a solution of 4-(4-(4-chloroquinolin-6-yl)-3-fluorobenzoyl)-1-methylpiperazin-2-one (100 mg, 0.251 mmol, 1.0 equiv) in ethanol (5 mL) was added benzo[d]thiazol-5-amine (56 mg, 0.377 mmol, 1.5 equiv) and TFA (0.1 mL). The reaction mixture was heated at 120° C. for 16 h. After completion of reaction, reaction mixture was concentrated under vacuum. The residue was neutralized by saturated sodium bicarbonate solution (20 mL), was extracted with ethyl acetate (2×40 mL), dried over anhydrous sodium sulphate, filtered and concentrated under vacuum to afford crude which was purified by flash chromatography to afford 4-(4-(4-(benzo[d]thiazol-5-ylamino)quinolin-6-yl)-3-fluorobenzoyl)-1-methylpiperazin-2-one (11 mg, 9%), LCMS: 512.1[M+1]⁺. ¹H NMR (400 MHz, DMSO-d₆) δ 2.88 (s, 3H) 3.64 (br. s., 2H) 3.89 (br. s., 2H) 4.02 (br. s., 1H) 4.18 (br. s., 1H) 6.96 (d, J=6.36 Hz, 1H) 7.49-7.58 (m, 2H) 7.63 (dd, J=8.27, 1.91 Hz, 1H) 7.85 (t, J=7.95 Hz, 1H) 8.12 (d, J=8.90 Hz, 1H) 8.22-8.31 (m, 1H) 8.41 (d, J=8.27 Hz, 1H) 8.58 (d, J=6.99 Hz, 1H) 8.99 (s, 1H) 9.54 (s, 1H) 11.10 (br. s., 1H) 14.29 (br. s., 1H).

### Example 68: Synthesis of 4-(4-(4-(benzo[d]thiazol-5-ylamino)quinolin-7-yl)-3-fluorobenzoyl)-1-methylpiperazin-2-one (Compound 274)

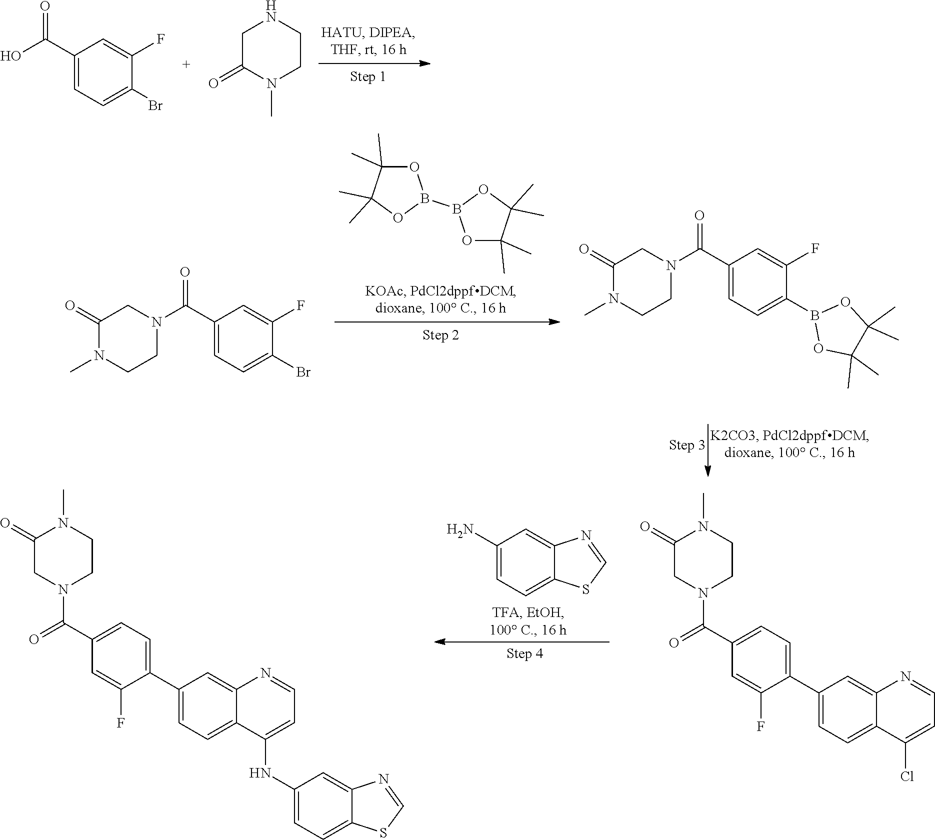

### Step-1: Synthesis of 4-(4-bromo-3-fluorobenzoyl)-1-methylpiperazin-2-one

To a solution of 4-bromo-3-fluorobenzoic acid (200 mg, 0.913 mmol, 1 equiv) in THF (5 mL) was added EDC·HCl (227 mg, 1.18 mmol, 1.3 equiv) and HOBT (160 mg, 1.18 mmol, 1.3 equiv). The reaction mixture was stirred at RT for 1 h. Then DIPEA (0.5 mL, 2.73 mmol, 3 equiv) was added followed by the addition of 1-methylpiperazin-2-one (125 mg, 1.09 mmol, 1.2 equiv). The reaction was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under vacuum to afford crude which was purified by flash chromatography to afford 4-(4-bromo-3-fluorobenzoyl)-1-methylpiperazin-2-one (280 mg, 97.5%), LCMS: 317.1[M+1]⁺.

### Step-2: Synthesis of 4-(3-fluoro-4-(4,4,5,5-tetramethyl-1,3,2-dioxaborolan-2-yl)benzoyl)-1-methylpiperazin-2-one

To a solution 4-(4-bromo-3-fluorobenzoyl)-1-methylpiperazin-2-one (280 mg, 0.89 mmol, 1.0 equiv) in 1,4-dioxane (5 mL) was added KOAc (261 mg, 2.66 mmol, 3.0 equiv) and 4,4,4′,4′,5,5,5′,5′-octamethyl-2,2′-bi(1,3,2-dioxaborolane) (270 mg, 1.066 mmol, 1.2 equiv). The reaction mixture was purged with nitrogen for 5 min, was added PdCl₂dppf·DCM (36 mg, 0.044 mmol, 0.05 equiv). Then the reaction was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under vacuum to afford crude which was purified by flash chromatography to afford 4-(3-fluoro-4-(4,4,5,5-tetramethyl-1,3,2-dioxaborolan-2-yl)benzoyl)-1-methylpiperazin-2-one (315 mg, 98%), LCMS: 281.2[M+1]⁺.[Boronic acid fragment].

### Step-3: Synthesis of 4-(4-(4-chloroquinolin-7-yl)-3-fluorobenzoyl)-1-methylpiperazin-2-one

To a solution of 4-(3-fluoro-4-(4,4,5,5-tetramethyl-1,3,2-dioxaborolan-2-yl)benzoyl)-1-methylpiperazin-2-one (100 mg, 0.276 mmol, 1 equiv) in 1,4-dioxane:water (3.5 mL:1.5 mL) was added K₂CO₃ (76 mg, 0.552 mmol, 2 equiv) and 7-bromo-4-chloroquinoline (66 mg, 0.276 mmol, 1 equiv). Reaction mixture was purged with nitrogen for 5 min, was added PdCl₂dppf·DCM (11 mg, 0.013 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography to afford 4-(4-(4-chloroquinolin-7-yl)-3-fluorobenzoyl)-1-methylpiperazin-2-one (73 mg, 66%), LCMS: 398.1[M+1]⁺.

### Step-4: Synthesis of 4-(4-(4-(benzo[d]thiazol-5-ylamino)quinolin-7-yl)-3-fluorobenzoyl)-1-methylpiperazin-2-one

To a solution of 4-(4-(4-chloroquinolin-7-yl)-3-fluorobenzoyl)-1-methylpiperazin-2-one (100 mg, 0.251 mmol, 1.0 equiv) in ethanol (5 mL) was added benzo[d]thiazol-5-amine (56 mg, 0.377 mmol, 1.5 equiv) and TFA (0.1 mL). The reaction mixture was heated at 120° C. for 16 h. After completion of reaction, reaction mixture was concentrated under vacuum to obtain crude which was neutralized by saturated sodium bicarbonate solution (20 mL), was extracted with ethyl acetate (2×40 mL), dried over anhydrous sodium sulphate, filtered and concentrated under vacuum to afford crude which was purified by flash chromatography to afford 4-(4-(4-(benzo[d]thiazol-5-ylamino)quinolin-7-yl)-3-fluorobenzoyl)-1-methylpiperazin-2-one (19 mg, 14.8%), LCMS: 512.1[M+1]⁺. ¹H NMR (400 MHz, DMSO-d₆) δ 2.89 (s, 3H) 3.37-3.44 (m, 3H) 3.68 (br. s., 2H) 4.17 (br. s., 1H) 7.04 (d, J=5.09 Hz, 1H) 7.44-7.58 (m, 3H) 7.79-7.86 (m, 2H) 8.11 (s, 1H) 8.06 (s, 1H) 8.22 (d, J=8.90 Hz, 1H) 8.54 (d, J=6.99 Hz, 2H) 9.41 (br. s., 1H) 9.44 (s, 1H).

### Example 69: Synthesis of 4-(4-(4-(benzo[d]thiazol-5-ylamino)quinolin-6-yl)-3-fluorobenzoyl)piperazin-2-one. (Compound 275)

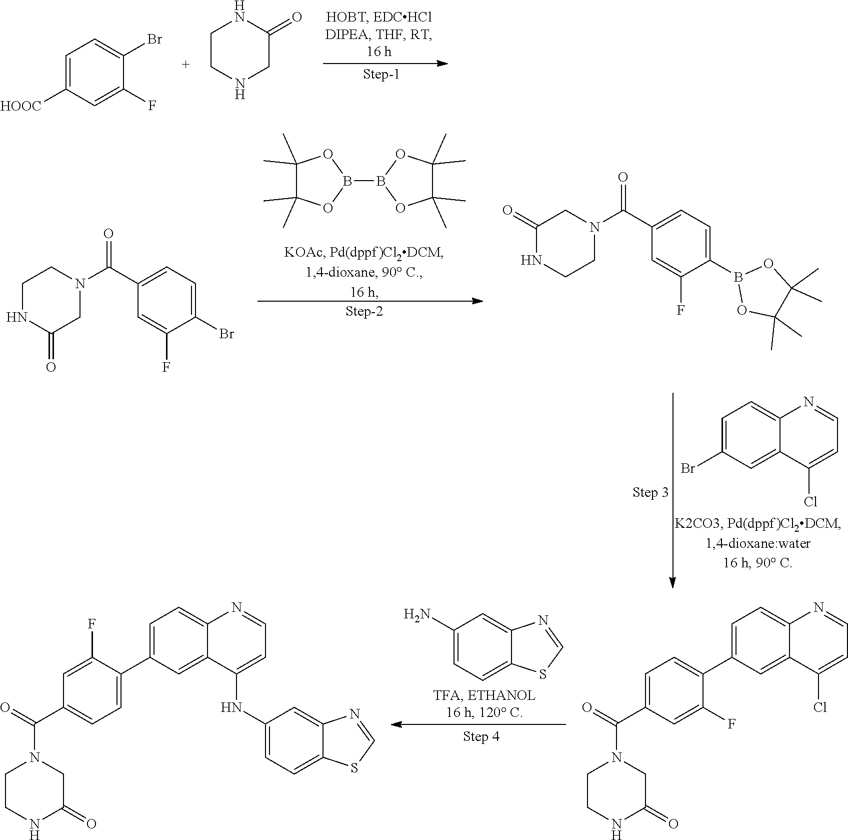

### Step-1: Synthesis of 4-(4-bromo-3-fluorobenzoyl)piperazin-2-one

To a stirred solution of 4-bromo-3-fluorobenzoic acid (500 mg, 2.29 mmol, 1.0 equiv) in THF (7 mL), was added HOBT (402.3 mg, 2.97 mmol, 1.3 equiv) and EDC·HCl (570.6 mg, 2.97 mmol, 1.3 equiv). The reaction was stirred at RT for 1 h. Then DIPEA (0.79 mL, 4.58 mmol, 2.0 equiv) and piperazin-2-one (274.2 mg, 2.73 mmol, 1.2 equiv) were added. Resulting reaction mixture was stirred at RT for 16 h. The progress of the reaction was monitored by TLC and LCMS. After completion of reaction, reaction mixture was concentrated under reduced pressure, diluted with water (75 mL) and extracted with EtOAc (2×100 mL). Combined organic layers were dried over anhydrous Na₂SO₄, filtered and concentrated under reduced pressure to afford 4-(4-bromo-3-fluorobenzoyl)piperazin-2-one (600 mg, 87.3%), LCMS: 301[M+1]⁺.

### Step-2: Synthesis of 4-(3-fluoro-4-(4,4,5,5-tetramethyl-1,3,2-dioxaborolan-2-yl)benzoyl)piperazin-2-one

To a stirred solution of 4-(4-bromo-3-fluorobenzoyl)piperazin-2-one (260 mg, 0.863 mmol, 1.0 equiv) and Bis(pinacolato)diboron (263.1 mg, 1.037 mmol, 1.2 equiv) in 1,4-Dioxane (5 mL) was added KOAc (254.1 mg, 2.589 mmol, 3.0 equiv). The reaction was mixture was purged with N₂ for 10 minutes, was added Pd(dppf)Cl₂·DCM (35.3 mg, 0.044 mmol, 0.05 equiv). The resulting reaction mixture was again purged with N₂ for 10 minutes and was stirred at 100° C. for 16 h. The progress of the reaction was monitored by LCMS and TLC. After completion of the reaction, reaction mixture was diluted with water (50 mL) and was extracted with EtOAc (2×75 mL). Combined organic layers were dried over Na₂SO₄, filtered and concentrated under reduced pressure to afford 4-(3-fluoro-4-(4,4,5,5-tetramethyl-1,3,2-dioxaborolan-2-yl)benzoyl)piperazin-2-one (245 mg, 81.3%), LCMS: 268.3[M+1]⁺.

### Step-3: Synthesis of 4-(4-(4-chloroquinolin-6-yl)-3-fluorobenzoyl)piperazin-2-one

To a stirred solution of 6-bromo-4-chloroquinoline (140 mg, 0.578 mmol, 1.0 equiv) and 4-(3-fluoro-4-(4,4,5,5-tetramethyl-1,3,2-dioxaborolan-2-yl)benzoyl)piperazin-2-one (241.2 mg, 0.693 mmol, 1.2 equiv) in 1,4-dioxane:water (4 mL:0.5 mL) was added K₂CO₃ (159.6 mg, 1.156 mmol, 2.0 equiv). The reaction mixture was purged with N2 gas for 10 minutes and was added Pd(dppf)Cl2·DCM (23.6 mg, 0.0289 mmol, 0.05 equiv) and the reaction mixture was heated at 90° C. for 16 h. The progress of the reaction was monitored by LCMS and TLC. After completion of reaction, reaction mixture was diluted with water (50 mL) and was extracted with EtOAc (2×100 mL), combined organic layers were dried over Na₂SO₄, filtered and concentrated under reduced pressure to afford crude which was purified by flash chromatography (eluted in 2% MeOH:DCM) to afford 4-(4-(4-chloroquinolin-6-yl)-3-fluorobenzoyl)piperazin-2-one (70 mg, 31.54%), LCMS: 384.3 [M+1]⁺.

### Step-4: Synthesis of 4-(4-(4-(benzo[d]thiazol-5-ylamino)quinolin-6-yl)-3-fluorobenzoyl)piperazin-2-one

To a solution of 4-(4-(4-chloroquinolin-6-yl)-3-fluorobenzoyl)piperazin-2-one (70 mg, 0.183 mmol, 1.0 equiv) and benzo[d]thiazol-5-amine (27.4 mg, 0.183 mmol, 1.0 equiv) in EtOH (2 mL) was added TFA (0.07 mL) and the reaction mixture was stirred at 90° C. for 16 h. The progress of reaction was monitored by TLC and LCMS. After completion of reaction, reaction mixture was concentrated under. The resulting residue was neutralized with saturated NaHCO₃ solution and was extracted with DCM (2×35 mL). Combined organic layers were dried over Na2SO4, filtered and concentrated under reduced pressure to afford crude which was purified by flash chromatography (eluted in 6% MeOH:DCM) to afford 4-(4-(4-(benzo[d]thiazol-5-ylamino)quinolin-6-yl)-3-fluorobenzoyl)piperazin-2-one (15 mg, 16.6%), LCMS: 498.1 [M+1]⁺, ¹H NMR (400 MHz, DMSO-d₆) δ 9.44 (s, 1H) 9.31 (br. s., 1H) 8.68 (s, 1H) 8.53 (d, J=5.09 Hz, 1H) 8.22 (d, J=8.27 Hz, 1H) 8.17 (br. s., 1H) 8.05 (s, 1H) 7.92-8.03 (m, 2H) 7.83 (t, J=7.95 Hz, 1H) 7.44-7.61 (m, 3H) 7.05 (d, J=4.45 Hz, 1H) 4.1 (br. s., 2H) 3.9 (br. s., 2H) 3.5 (br. s., 2H).

### Example 70: Synthesis of 4-(4-(4-(benzo[d]thiazol-5-ylamino)quinolin-7-yl)-3-fluorobenzoyl)piperazin-2-one (Compound 276)

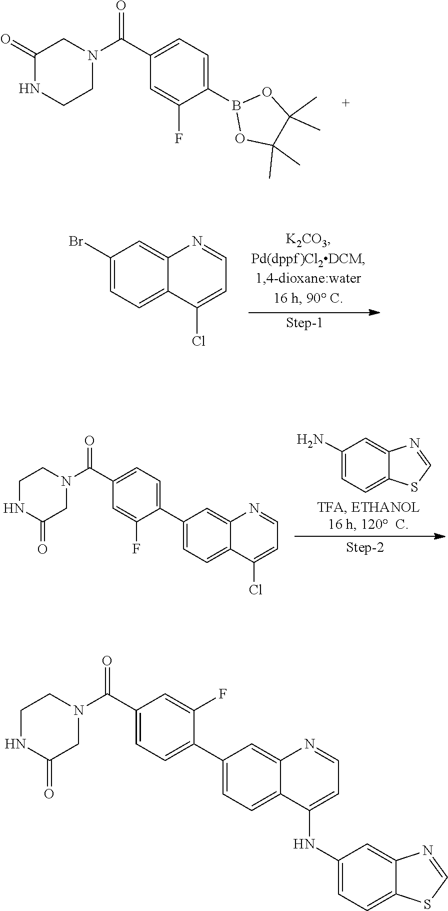 [SMILES: CC1(C)OB(c2ccc(C(=O)N3CCNC(=O)C3)cc2F)OC1(C)C ; Clc1ccnc2cc(Br)ccc12 ; O=C1CN(C(=O)c2ccc(-c3ccc4c(Cl)ccnc4c3)c(F)c2)CCN1 ; Nc1ccc2scnc2c1 ; O=C1CN(C(=O)c2ccc(-c3ccc4c(Nc5ccc6scnc6c5)ccnc4c3)c(F)c2)CCN1]

### Step-1: Synthesis of 4-(4-(4-chloroquinolin-7-yl)-3-fluorobenzoyl)piperazin-2-one

To a stirred solution of 7-bromo-4-chloroquinoline (140 mg, 0.578 mmol, 1.0 equiv) and 4-(3-fluoro-4-(4,4,5,5-tetramethyl-1,3,2-dioxaborolan-2-yl)benzoyl)piperazin-2-one (241.2 mg, 0.693 mmol, 1.2 equiv) in 1,4-dioxane:water (4 mL:0.5 mL) was added K₂CO₃ (159.6 mg, 1.156 mmol, 2.0 equiv). The reaction mixture was purged with N2 gas for 10 minutes and was added Pd(dppf)Cl2·DCM (23.6 mg, 0.0289 mmol, 0.05 equiv). Reaction mixture was heated at 90° C. for 16 h. The progress of the reaction was monitored by LCMS and TLC. After completion of reaction, reaction mixture was diluted with water (50 mL) and was extracted with EtOAc (2×100 mL), combined organic layers were dried over Na₂SO₄, filtered and concentrated under reduced pressure to afford crude which was purified by flash chromatography (eluted in 2% MeOH:DCM) to afford 4-(4-(4-chloroquinolin-7-yl)-3-fluorobenzoyl)piperazin-2-one (119 mg, 41%), LCMS: 355.2 [M+1]⁺.

### Step-2: Synthesis of 4-(4-(4-(benzo[d]thiazol-5-ylamino)quinolin-7-yl)-3-fluorobenzoyl)piperazin-2-one

To a solution of 4-(4-(4-chloroquinolin-7-yl)-3-fluorobenzoyl)piperazin-2-one (70 mg, 0.183 mmol, 1.0 equiv) and benzo[d]thiazol-5-amine (27.4 mg, 0.183 mmol, 1.0 equiv) in EtOH (2 mL) was added TFA (0.07 mL) and the reaction mixture was stirred at 90° C. for 16 h. The progress of reaction was monitored by TLC and LCMS. After completion of reaction, reaction mixture was concentrated under reduced pressure to obtain crude which was neutralized with saturated NaHCO₃ solution (25 mL) and was extracted with DCM (2×35 mL). Combined organic layers were dried over Na2SO4, filtered and concentrated under reduced pressure to afford crude which was purified by flash chromatography (eluted in 6% MeOH:DCM) to afford 4-(4-(4-(benzo[d]thiazol-5-ylamino)quinolin-7-yl)-3-fluorobenzoyl)piperazin-2-one (12 mg, 13%), LCMS: 498.1 [M+1]⁺, ¹H NMR (400 MHz, DMSO-d₆) δ 9.42 (s, 1H) 9.31 (br. s., 1H) 8.58 (s, 1H) 8.53 (d, J=5.09 Hz, 1H) 8.12 (d, J=8.27 Hz, 1H) 8.08 (br. s., 1H) 7.9 (s, 1H) 7.80-8.03 (m, 2H) 7.73 (t, J=7.95 Hz, 1H) 7.34-7.51 (m, 3H) 7.01 (d, J=4.45 Hz, 1H) 3.9 (br. s., 2H) 3.5 (br. s., 2H) 3.2 (br. s., 2H).

### Example 71: 4-(4-(4-(1H-indazol-5-ylamino)quinolin-6-yl)-3-fluorobenzyl)piperazin-2-one (Compound 271)

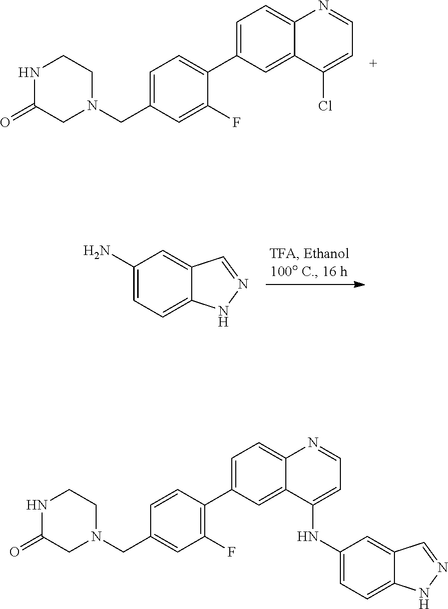 [SMILES: O=C1CN(Cc2ccc(-c3ccc4nccc(Cl)c4c3)c(F)c2)CCN1 ; Nc1ccc2[nH]ncc2c1 ; O=C1CN(Cc2ccc(-c3ccc4nccc(Nc5ccc6[nH]ncc6c5)c4c3)c(F)c2)CCN1]

To a solution of 4-(4-(4-chloroquinolin-6-yl)-3-fluorobenzyl)piperazin-2-one (50 mg, 0.14 mmol, 1.0 equiv) and 1H-indazol-5-amine (18 mg, 0.14 mmol, 1.0 equiv) in EtOH (5 mL) was added TFA (0.05 mL) and the reaction mixture was allowed to reflux at 100° C. for 16 h. The progress of the reaction was monitored by TLC and LCMS. After completion of reaction, reaction mixture was concentrated under reduced pressure and was neutralized with saturated NaHCO₃. The aqueous phase was extracted with DCM (2×30 mL). Combined organic layer was dried over anhydrous Na₂SO₄, filtered and concentrated under reduced pressure to afford crude product which was purified by flash chromatography, eluted in 6% MeOH:DCM to afford 4-(4-(4-(1H-indazol-5-ylamino)quinolin-6-yl)-3-fluorobenzyl)piperazin-2-one (30 mg, 48%). LCMS: 467 [M+1]⁺, 1H NMR (400 MHz, DMSO-d₆) δ 8.70 (br. s., 2H), 8.43 (d, J=5.7 Hz, 2H), 8.10 (s, 1H), 7.95 (s, 2H), 7.79-7.71 (m, 2H), 7.71-7.58 (m, 2H), 7.47-7.20 (m, 3H), 6.72 (d, J=6.4 Hz, 1H), 3.64 (s, 2H), 3.19 (br. s., 1H), 2.96 (d, 2H), 2.61 (d, J=5.1 Hz, 2H).

### Example 72: 4-(4-(4-(benzo[b]thiophen-5-ylamino)quinolin-6-yl)-3-fluorobenzyl)piperazin-2-one (Compound 256)

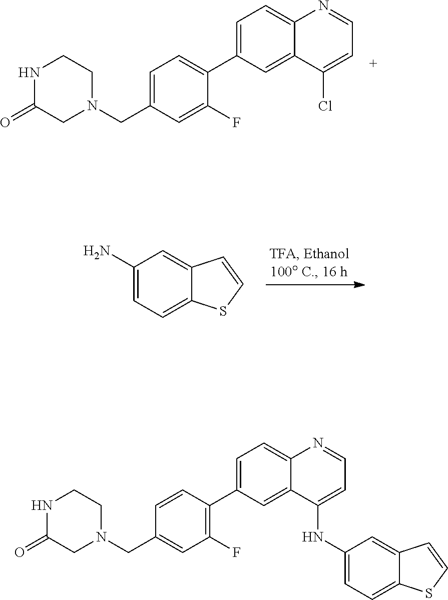 [SMILES: O=C1CN(Cc2ccc(-c3ccc4nccc(Cl)c4c3)c(F)c2)CCN1 ; Nc1ccc2sccc2c1 ; O=C1CN(Cc2ccc(-c3ccc4nccc(Nc5ccc6sccc6c5)c4c3)c(F)c2)CCN1]

To a solution of 4-(4-(4-chloroquinolin-6-yl)-3-fluorobenzyl)piperazin-2-one (50 mg, 0.14 mmol, 1.0 equiv) and benzo[b]thiophen-5-amine (20 mg, 0.14 mmol, 1.0 equiv) in EtOH (5 mL) was added TFA (0.05 mL) and the reaction mixture was allowed to reflux at 100° C. for 16 h. The progress of the reaction was monitored by TLC and LCMS. After completion of reaction, reaction mixture was concentrated under reduced pressure and was neutralized with saturated NaHCO₃. The aqueous phase was extracted with DCM (2×30 mL). Combined organic layer was dried over anhydrous Na₂SO₄, filtered and concentrated under reduced pressure to afford crude product which was purified by flash chromatography, eluted in 6% MeOH:DCM to afford 4-(4-(4-(benzo[b]thiophen-5-ylamino)quinolin-6-yl)-3-fluorobenzyl)piperazin-2-one (30 mg, 46%). LCMS: 467 [M+1]⁺, 1H NMR (400 MHz, DMSO-d₆) δ 8.70 (br. s., 2H), 8.43 (d, J=5.7 Hz, 2H), 8.10 (s, 1H), 7.95 (s, 2H), 7.79-7.71 (m, 2H), 7.71-7.58 (m, 2H), 7.47-7.20 (m, 3H), 6.72 (d, J=6.4 Hz, 1H), 3.64 (s, 2H), 3.19 (br. s., 1H), 2.96 (d, 2H), 2.61 (d, J=5.1 Hz, 2H).

### Example 73: Synthesis of (4-(4-(benzo[d]thiazol-5-ylamino)quinolin-6-yl)-3-fluorophenyl)(7-oxa-2-azaspiro[3.5]nonan-2-yl)methanone (Compound 251)

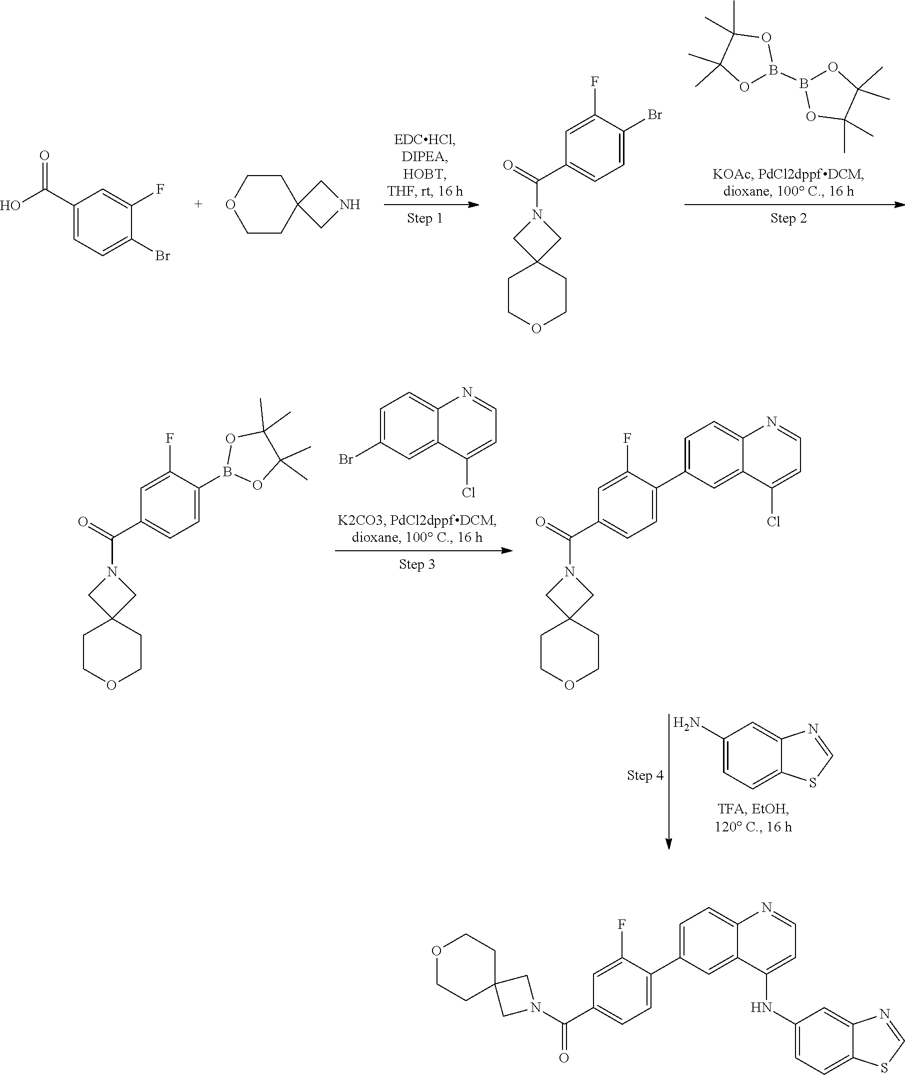

### Step-1: Synthesis of (4-bromo-3-fluorophenyl)(7-oxa-2-azaspiro[3.5]nonan-2-yl)methanone

To a solution of 4-bromo-3-fluorobenzoic acid (200 mg, 0.91 mmol, 1.0 equiv) in THF (5 mL) was added EDC·HCl (227 mg, 1.18 mmol, 1.3 equiv) and HOBT (160 mg, 1.18 mmol, 1.3 equiv). Reaction was stirred at RT for 1 h. Then added, DIPEA (0.5 mL, 2.73 mmol, 3 equiv) and 7-oxa-2-azaspiro[3.5]nonane (139 mg, 1.09 mmol, 1.2 equiv). Reaction was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under vacuum to afford crude product which was purified by flash chromatography to afford (4-bromo-3-fluorophenyl)(7-oxa-2-azaspiro[3.5]nonan-2-yl)methanone (270 mg, 90%), LCMS: 328.1[M+1]⁺.

### Step-2: Synthesis of (3-fluoro-4-(4,4,5,5-tetramethyl-1,3,2-dioxaborolan-2-yl)phenyl)(7-oxa-2-azaspiro[3.5]nonan-2-yl)methanone

To a solution (4-bromo-3-fluorophenyl)(7-oxa-2-azaspiro[3.5]nonan-2-yl)methanone (270 mg, 0.825 mmol, 1.0 equiv) in 1,4-dioxane (8 mL) was added KOAc (242 mg, 2.47 mmol, 3.0 equiv) and 4,4,4′,4′,5,5,5′,5′-octamethyl-2,2′-bi(1,3,2-dioxaborolane) (250 mg, 0.99 mmol, 1.2 equiv). Reaction mixture was purged with nitrogen for 5 min, and then was added PdCl₂dppf·DCM (33 mg, 0.041 mmol, 0.05 equiv). The reaction was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under vacuum to afford crude product which was purified by flash chromatography to afford (3-fluoro-4-(4,4,5,5-tetramethyl-1,3,2-dioxaborolan-2-yl)phenyl)(7-oxa-2-azaspiro[3.5]nonan-2-yl)methanone (300 mg, 97%), LCMS: 294.2[M+1]⁺[Boronic-acid Fragment].

### Step-3: Synthesis of (4-(4-chloroquinolin-6-yl)-3-fluorophenyl)(7-oxa-2-azaspiro[3.5]nonan-2-yl)methanone

To a solution of (3-fluoro-4-(4,4,5,5-tetramethyl-1,3,2-dioxaborolan-2-yl)phenyl)(7-oxa-2-azaspiro[3.5]nonan-2-yl)methanone (150 mg, 0.4 mmol, 1 equiv) in 1,4-dioxane:water (3.5 mL:1.5 mL) was added K₂CO₃ (110 mg, 0.8 mmol, 2.0 equiv) and 6-bromo-4-chloroquinoline (97 mg, 0.4 mmol, 1.0 equiv). Reaction mixture was purged with nitrogen for 5 min, and then was added PdCl₂dppf·DCM (16 mg, 0.02 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography to afford (4-(4-chloroquinolin-6-yl)-3-fluorophenyl)(7-oxa-2-azaspiro[3.5]nonan-2-yl)methanone (50 mg, 30%), LCMS: 411.2[M+1]⁺.

### Step-4: Synthesis of (4-(4-(benzo[d]thiazol-5-ylamino)quinolin-6-yl)-3-fluorophenyl)(7-oxa-2-azaspiro[3.5]nonan-2-yl)methanone

To a solution of (4-(4-chloroquinolin-6-yl)-3-fluorophenyl)(7-oxa-2-azaspiro[3.5]nonan-2-yl)methanone (50 mg, 0.12 mmol, 1.0 equiv) in ethanol (5 mL) was added benzo[d]thiazol-5-amine (18 mg, 0.12 mmol, 1.0 equiv) and TFA (0.05 mL). The reaction mixture was heated at 120° C. for 16 h. After completion of reaction, reaction mixture was concentrated under vacuum to obtain a residue which was neutralized by saturated sodium bicarbonate solution (10 mL), was extracted with ethyl acetate (2×20 mL), dried over anhydrous sodium sulphate, filtered and concentrated under vacuum to afford crude which was purified by flash chromatography to afford (4-(4-(benzo[d]thiazol-5-ylamino)quinolin-6-yl)-3-fluorophenyl)(7-oxa-2-azaspiro[3.5]nonan-2-yl)methanone (10 mg, 15%), LCMS: 525.1[M+1]. ¹H NMR (400 MHz, DMSO-d₆) δ 1.70-1.76 (m, 4H) 3.44-3.52 (m, 2H) 3.52-3.60 (m, 2H) 3.82 (s, 2H) 4.15 (s, 2H) 7.03 (d, J=5.72 Hz, 2H) 7.57 (dd, J=8.27, 1.91 Hz, 1H) 7.62-7.68 (m, 2H) 7.80-7.86 (m, 1H) 7.99-8.04 (m, 2H) 8.09 (d, J=1.91 Hz, 1H) 8.26 (d, J=8.90 Hz, 1H) 8.53 (d, J=5.72 Hz, 1H) 8.75 (s, 1H) 9.46 (s, 1H).

### Example 74: Synthesis of (4-(4-(benzo[d]thiazol-5-ylamino)quinolin-7-yl)-3-fluorophenyl)(7-oxa-2-azaspiro[3.5]nonan-2-yl)methanone (Compound 252)

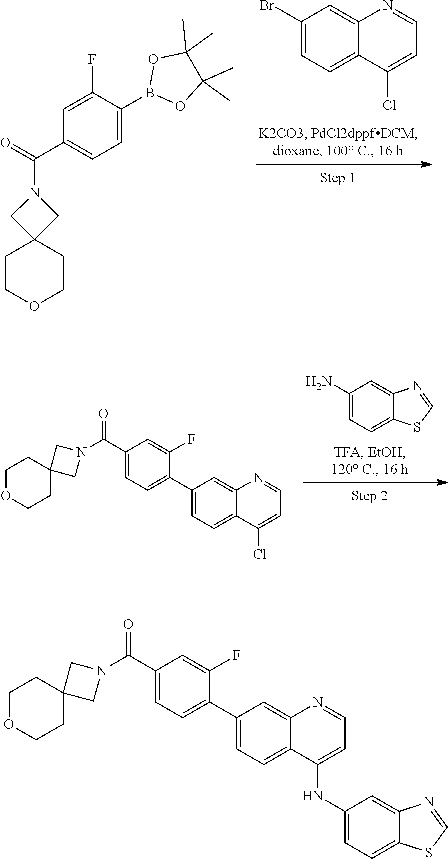 [SMILES: CC1(C)OB(c2ccc(C(=O)N3CC4(CCOCC4)C3)cc2F)OC1(C)C ; Clc1ccnc2cc(Br)ccc12 ; O=C(c1ccc(-c2ccc3c(Cl)ccnc3c2)c(F)c1)N1CC2(CCOCC2)C1 ; Nc1ccc2scnc2c1 ; O=C(c1ccc(-c2ccc3c(Nc4ccc5scnc5c4)ccnc3c2)c(F)c1)N1CC2(CCOCC2)C1]

### Step-1: Synthesis of (4-(4-chloroquinolin-7-yl)-3-fluorophenyl)(7-oxa-2-azaspiro[3.5]nonan-2-yl)methanone

To a solution of (3-fluoro-4-(4,4,5,5-tetramethyl-1,3,2-dioxaborolan-2-yl)phenyl)(7-oxa-2-azaspiro[3.5]nonan-2-yl)methanone (150 mg, 0.4 mmol, 1.0 equiv) in 1,4-dioxane:water (3.5 mL:1.5 mL) was added K₂CO₃ (110 mg, 0.8 mmol, 2.0 equiv) and 7-bromo-4-chloroquinoline (97 mg, 0.4 mmol, 1.0 equiv). Reaction mixture was purged with nitrogen for 5 min, then was added PdCl₂dppf·DCM (16 mg, 0.02 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography to afford (4-(4-chloroquinolin-7-yl)-3-fluorophenyl)(7-oxa-2-azaspiro[3.5]nonan-2-yl)methanone (45 mg, 27%), LCMS: 411.2[M+1]⁺.

### Step-2: Synthesis of (4-(4-(benzo[d]thiazol-5-ylamino)quinolin-7-yl)-3-fluorophenyl)(7-oxa-2-azaspiro[3.5]nonan-2-yl)methanone

To a solution of (4-(4-chloroquinolin-7-yl)-3-fluorophenyl)(7-oxa-2-azaspiro[3.5]nonan-2-yl)methanone (45 mg, 0.11 mmol, 1.0 equiv) in ethanol (5 mL) was added benzo[d]thiazol-5-amine (16 mg, 0.11 mmol, 1.0 equiv) and TFA (0.05 mL). The reaction mixture was heated at 120° C. for 16 h. After completion of reaction, reaction mixture was concentrated under vacuum to obtain residue which was neutralized by saturated sodium bicarbonate solution (10 mL), was extracted with ethyl acetate (2×20 mL), dried over anhydrous sodium sulphate, filtered and concentrated under vacuum to afford crude which was purified by flash chromatography to afford (4-(4-(benzo[d]thiazol-5-ylamino)quinolin-7-yl)-3-fluorophenyl)(7-oxa-2-azaspiro[3.5]nonan-2-yl)methanone (10 mg, 17%), LCMS: 525.1[M+1]. ¹H NMR (400 MHz, DMSO-d₆) δ 1.70-1.76 (m, 5H) 3.44-3.52 (m, 3H) 3.52-3.60 (m, 2H) 3.82 (s, 2H) 4.15 (s, 2H) 7.03 (d, J=5.72 Hz, 1H) 7.57 (dd, J=8.27, 1.91 Hz, 1H) 7.62-7.68 (m, 2H) 7.80-7.86 (m, 1H) 7.99-8.04 (m, 2H) 8.09 (d, J=1.91 Hz, 1H) 8.26 (d, J=8.90 Hz, 1H) 8.53 (d, J=5.72 Hz, 1H) 8.75 (s, 1H) 9.46 (s, 1H).

### Example 75: Synthesis of (4-(4-(benzo[d]thiazol-5-ylamino)quinolin-6-yl)-3-fluorophenyl)(piperazin-1-yl)methanone (Compound 249)

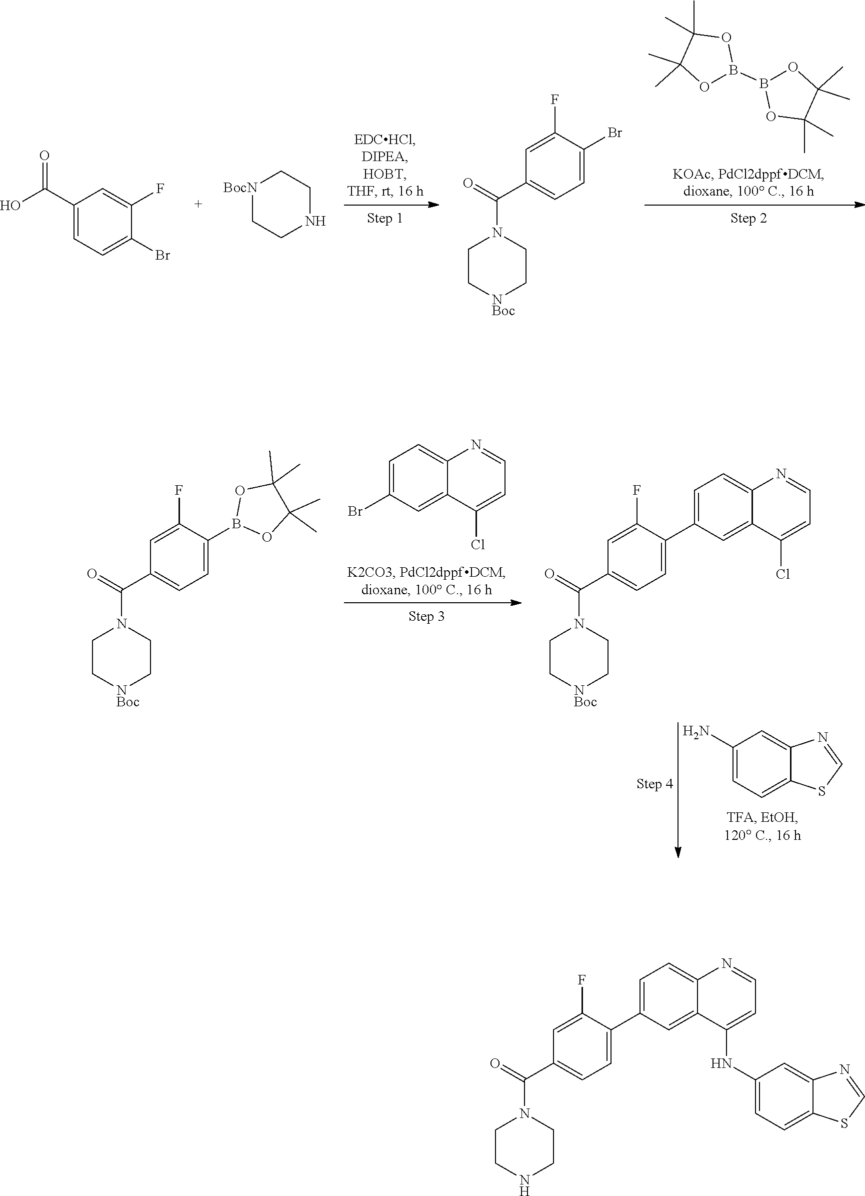

### Step-1: Synthesis of tert-butyl 4-(4-bromo-3-fluorobenzoyl)piperazine-1-carboxylate

To a solution of 4-bromo-3-fluorobenzoic acid (200 mg, 0.91 mmol, 1.0 equiv) in THF (5 mL) was added EDC·HCl (227 mg, 1.18 mmol, 1.3 equiv) and HOBT (160 mg, 1.18 mmol, 1.3 equiv). Reaction was stirred at RT for 1 h. Then added, DIPEA (0.5 mL, 2.73 mmol, 3 equiv) and 7-oxa-2-azaspiro[3.5]nonane (203 mg, 1.09 mmol, 1.2 equiv). Reaction was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under vacuum to afford crude which was purified by flash chromatography to afford tert-butyl 4-(4-bromo-3-fluorobenzoyl)piperazine-1-carboxylate (350 mg, 99%). LCMS: 387.2[M+1]⁺

### Step-2: Synthesis of tert-butyl 4-(3-fluoro-4-(4,4,5,5-tetramethyl-1,3,2-dioxaborolan-2-yl)benzoyl)piperazine-1-carboxylate

To a solution tert-butyl 4-(4-bromo-3-fluorobenzoyl)piperazine-1-carboxylate (350 mg, 0.90 mmol, 1.0 equiv) in 1,4-dioxane (10 mL) was added KOAc (265 mg, 2.71 mmol, 3.0 equiv) and 4,4,4′,4′,5,5,5′,5′-octamethyl-2,2′-bi(1,3,2-dioxaborolane) (275 mg, 1.08 mmol, 1.2 equiv). Reaction mixture was purged with nitrogen for 5 min, then was added PdCl₂dppf·DCM (36 mg, 0.04 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under vacuum to afford crude which was purified by flash chromatography to afford tert-butyl 4-(3-fluoro-4-(4,4,5,5-tetramethyl-1,3,2-dioxaborolan-2-yl)benzoyl)piperazine-1-carboxylate (380 mg, 96%), LCMS: 379.2[M+1]⁺[Boc-acid Fragment].

### Step-3: Synthesis of tert-butyl 4-(4-(4-chloroquinolin-6-yl)-3-fluorobenzoyl)piperazine-1-carboxylate

To a solution of tert-butyl 4-(3-fluoro-4-(4,4,5,5-tetramethyl-1,3,2-dioxaborolan-2-yl)benzoyl)piperazine-1-carboxylate (180 mg, 0.414 mmol, 1 equiv) in 1,4-dioxane:water (3.5 mL:1.5 mL) was added K₂CO₃ (114 mg, 0.829 mmol, 2 equiv) and 6-bromo-4-chloroquinoline (100 mg, 0.414 mmol, 1 equiv). Reaction mixture was purged with nitrogen for 5 min, was added PdCl₂dppf·DCM (16 mg, 0.020 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography to afford tert-butyl 4-(4-(4-chloroquinolin-6-yl)-3-fluorobenzoyl)piperazine-1-carboxylate (100 mg, 51%), LCMS: 470.1 [M+1]⁺.

### Step-4: Synthesis of (4-(4-(benzo[d]thiazol-5-ylamino)quinolin-6-yl)-3-fluorophenyl)(piperazin-1-yl)methanone

To a solution of tert-butyl 4-(4-(4-chloroquinolin-6-yl)-3-fluorobenzoyl)piperazine-1-carboxylate (100 mg, 0.213 mmol, 1.0 equiv) in ethanol (5 mL) was added benzo[d]thiazol-5-amine (31 mg, 0.213 mmol, 1.0 equiv) and TFA (0.1 mL). The reaction mixture was heated at 120° C. for 16 h. After completion of reaction, reaction mixture was concentrated under vacuum to obtain residue which was neutralized by saturated sodium bicarbonate solution (20 mL), was extracted with ethyl acetate (2×40 mL), dried over anhydrous sodium sulphate, filtered and concentrated under vacuum to afford crude which was purified by flash chromatography to afford (4-(4-(benzo[d]thiazol-5-ylamino)quinolin-6-yl)-3-fluorophenyl)(piperazin-1-yl)methanone (20 mg, 19%). LCMS: 484.1[M+1]. ¹HNMR (400 MHz, DMSO-d₆) δ 3.01 (br. s., 3H) 3.68 (br. s., 3H) 3.74 (br. s., 2H) 7.05 (br. s., 1H) 7.45 (br. s., 2H) 7.55 (d, J=9.54 Hz, 1H) 7.83 (s, 1H) 7.94 (br. s., 1H) 7.98 (br. s., 1H) 8.04 (br. s., 1H) 8.21 (d, J=8.27 Hz, 2H) 8.53 (br. s., 1H) 8.66 (br. s., 1H) 9.43 (s, 1H).

### Example 76: Synthesis of (4-(4-(benzo[d]thiazol-5-ylamino)quinolin-7-yl)-3-fluorophenyl)(piperazin-1-yl)methanone (Compound 250)

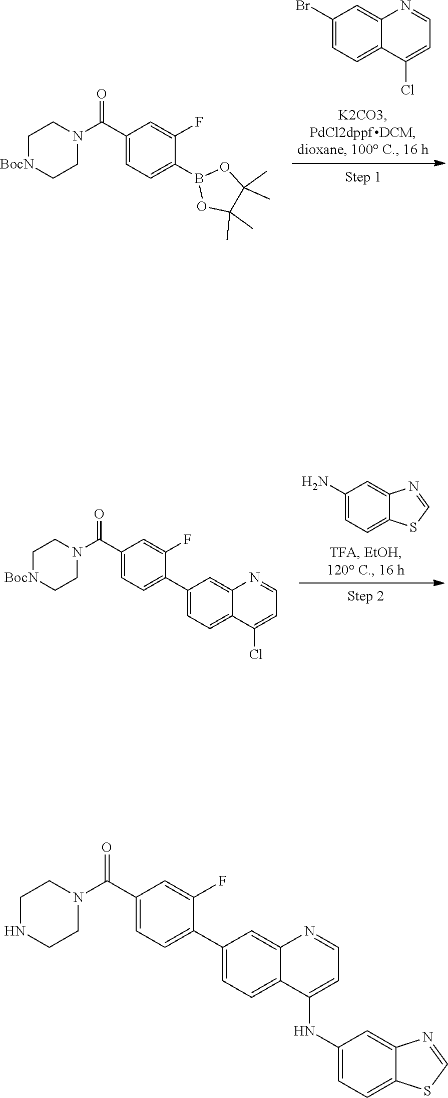 [SMILES: Clc1ccnc2cc(Br)ccc12 ; CC(C)(C)OC(=O)N1CCN(C(=O)c2ccc(B3OC(C)(C)C(C)(C)O3)c(F)c2)CC1 ; CC(C)(C)OC(=O)N1CCN(C(=O)c2ccc(-c3ccc4c(Cl)ccnc4c3)c(F)c2)CC1 ; Nc1ccc2scnc2c1 ; O=C(c1ccc(-c2ccc3c(Nc4ccc5scnc5c4)ccnc3c2)c(F)c1)N1CCNCC1]

### Step-1: Synthesis of tert-butyl 4-(4-(4-chloroquinolin-7-yl)-3-fluorobenzoyl)piperazine-1-carboxylate

To a solution of tert-butyl 4-(3-fluoro-4-(4,4,5,5-tetramethyl-1,3,2-dioxaborolan-2-yl)benzoyl)piperazine-1-carboxylate (150 mg, 0.34 mmol, 1.0 equiv) in 1,4-dioxane:water (3.5 mL:1.5 mL) was added K₂CO₃ (95 mg, 0.69 mmol, 2.0 equiv) and 7-bromo-4-chloroquinoline (83 mg, 0.34 mmol, 1.0 equiv). Reaction mixture was purged with nitrogen for 5 min, then was added PdCl₂dppf·DCM (14 mg, 0.02 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography to tert-butyl 4-(4-(4-chloroquinolin-7-yl)-3-fluorobenzoyl)piperazine-1-carboxylate (115 mg, 71%). LCMS: 470.1 [M+1]⁺.

### Step-2: Synthesis of (4-(4-(benzo[d]thiazol-5-ylamino)quinolin-7-yl)-3-fluorophenyl)(piperazin-1-yl)methanone

To a solution of tert-butyl 4-(4-(4-chloroquinolin-7-yl)-3-fluorobenzoyl)piperazine-1-carboxylate (115 mg, 0.24 mmol, 1.0 equiv) in ethanol (5 mL) was added benzo[d]thiazol-5-amine (36 mg, 0.245 mmol, 1.0 equiv) and TFA (0.1 mL). The reaction mixture was heated at 120° C. for 16 h. After completion of reaction, reaction mixture was concentrated under vacuum to obtain residue which was neutralized by saturated sodium bicarbonate solution (20 mL), was extracted with ethyl acetate (2×40 mL), dried over anhydrous sodium sulphate, filtered and concentrated under vacuum to afford crude which was purified by flash chromatography to afford (4-(4-(benzo[d]thiazol-5-ylamino)quinolin-7-yl)-3-fluorophenyl)(piperazin-1-yl)methanone (21 mg, 17%). LCMS: 484.1[M+1]. ¹HNMR (400 MHz, DMSO-d₆) δ 3.01 (br. s., 4H) 3.68 (br. s., 2H) 3.74 (br. s., 2H) 7.05 (br. s., 1H) 7.45 (br. s., 2H) 7.55 (d, J=9.54 Hz, 1H) 7.83 (s, 1H) 7.94 (br. s., 1H) 7.98 (br. s., 1H) 8.04 (br. s., 1H) 8.21 (d, J=8.27 Hz, 2H) 8.53 (br. s., 1H) 8.66 (br. s., 1H) 9.43 (s, 1H).

### Example 77: Synthesis of 1-(4-(4-(4-(benzo[d]thiazol-5-ylamino)quinolin-6-yl)-3-fluorobenzyl)piperazin-1-yl)-2-methylpropan-1-one (Compound 281)

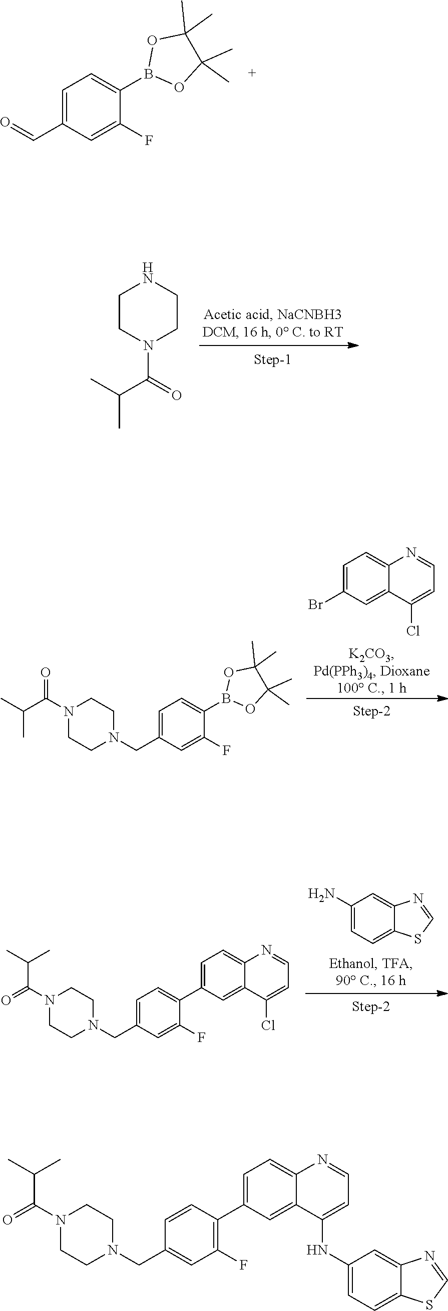

### Step-1: Synthesis of 1-(4-(3-fluoro-4-(4,4,5,5-tetramethyl-1,3,2-dioxaborolan-2-yl)benzyl)piperazin-1-yl)-2-methylpropan-1-one

To a stirred solution of 3-fluoro-4-(4,4,5,5-tetramethyl-1,3,2-dioxaborolan-2-yl)benzaldehyde (1 g, 4.0 mmol, 1.0 equiv) and 2-methyl-1-(piperazin-1-yl)propan-1-one (937 mg, 6.0 mmol, 1.5 equiv) in DCM (20 mL), was added acetic acid (0.45 mL, 8.0 mmol, 2.0 equiv) dropwise at 0° C. and reaction mixture was allowed to stirred at RT for 1 h. Sodium cyanoborohydride (377 mg, 6.0 mmol, 1.5 equiv) was added portion wise at 0° C. The resulting reaction mixture was stirred at RT for 16 h. The progress of the reaction was monitored by LCMS. After completion of reaction, reaction mixture was diluted with water (25 mL) and extracted with DCM (3×100 mL). Combined organic layer was washed with saturated NaHCO₃ (50 mL) solution, dried over anhydrous Na₂SO₄ and concentrated under reduced pressure to afford 1-(4-(3-fluoro-4-(4,4,5,5-tetramethyl-1,3,2-dioxaborolan-2-yl)benzyl)piperazin-1-yl)-2-methylpropan-1-one (900 mg, 60%) LCMS: 309.3[M+1]+(Boronic-acid Fragment).

### Step-2: Synthesis of 1-(4-(4-(4-chloroquinolin-6-yl)-3-fluorobenzyl)piperazin-1-yl)-2-methylpropan-1-one

To a stirred solution of 6-bromo-4-chloroquinoline (518 mg, 2.136 mmol, 1.0 equiv) and 1-(4-(3-fluoro-4-(4,4,5,5-tetramethyl-1,3,2-dioxaborolan-2-yl)benzyl)piperazin-1-yl)-2-methylpropan-1-one (1.0 g, 2.563 mmol, 1.2 equiv) in 1,4-dioxane (30 mL), was added K₂CO₃ 1M solution (17 mL). Reaction was purged with N₂ gas for 10 minutes and then was added Pd(PPh3)4 (246.8 mg, 0.213 mmol, 0.1 equiv). Resulting reaction mixture was stirred at 100° C. for 1 h. The progress of the reaction was monitored by LCMS and TLC. After completion reaction, reaction mixture was diluted with water (50 mL) and extracted with EtOAc (3×100 mL). Combined organic layers were dried by Na₂SO₄, filtered and concentrated under reduced pressure. The resulting crude was purified by flash chromatography (eluted in 1% MeOH:DCM) to afford 1-(4-(4-(4-chloroquinolin-6-yl)-3-fluorobenzyl)piperazin-1-yl)-2-methylpropan-1-one (650 mg, 71%) LCMS: 426.2 [M+1]⁺

### Step-3: Synthesis of 1-(4-(4-(4-(benzo[d]thiazol-5-ylamino)quinolin-6-yl)-3-fluorobenzyl)piperazin-1-yl)-2-methylpropan-1-one

To a solution of 1-(4-(4-(4-chloroquinolin-6-yl)-3-fluorobenzyl)piperazin-1-yl)-2-methylpropan-1-one (750 mg, 1.760 mmol, 1.0 equiv) and benzo[d]thiazol-5-amine (265 mg, 1.76 mmol, 1.0 equiv) in EtOH (10 mL) was added TFA (0.6 mL). The reaction mixture was allowed to heat at 90° C. for 16 h. The progress of the reaction was monitored by TLC and LCMS. After completion of reaction, reaction mixture was concentrated under reduced pressure. The resulting residue was neutralized with saturated solution of NaHCO₃ and was extracted with DCM (3×50 mL). Combined organic layer was dried over anhydrous Na₂SO₄, filtered and concentrated under reduced pressure. The crude product was purified by flash chromatography (eluted in 2% MeOH:DCM) to afford 1-(4-(4-(4-(benzo[d]thiazol-5-ylamino)quinolin-6-yl)-3-fluorobenzyl)piperazin-1-yl)-2-methylpropan-1-one (250 mg, 26%). LCMS: 540.2 [M+1]⁺, ¹H NMR (400 MHz, DMSO-d₆) δ 9.6 (br. s, 1H) 9.45 (s, 1H) 8.67 (s, 1H) 8.52 (d, J=5.09 Hz, 1H) 8.24 (d, J=8.90 Hz, 1H) 8.07 (d, J=1.27 Hz, 1H) 7.94-8.00 (m, 2H) 7.70 (t, J=7.95 Hz, 1H) 7.56 (dd, J=8.27, 1.91 Hz, 1H) 7.31-7.37 (m, 2H) 7.03 (d, J=5.72 Hz, 1H) 3.60 (s, 2H) 3.50 (br. s., 4H) 2.85 (quin, J=6.68 Hz, 1H) 2.42 (br. s., 2H) 2.36 (br. s., 2H) 0.98 (d, J=6.36 Hz, 6H).

### Example 78: Synthesis of N-(benzo[b]thiophen-5-yl)-6-(2-fluoro-4-(piperazin-1-ylmethyl)phenyl)quinolin-4-amine (Compound 385)

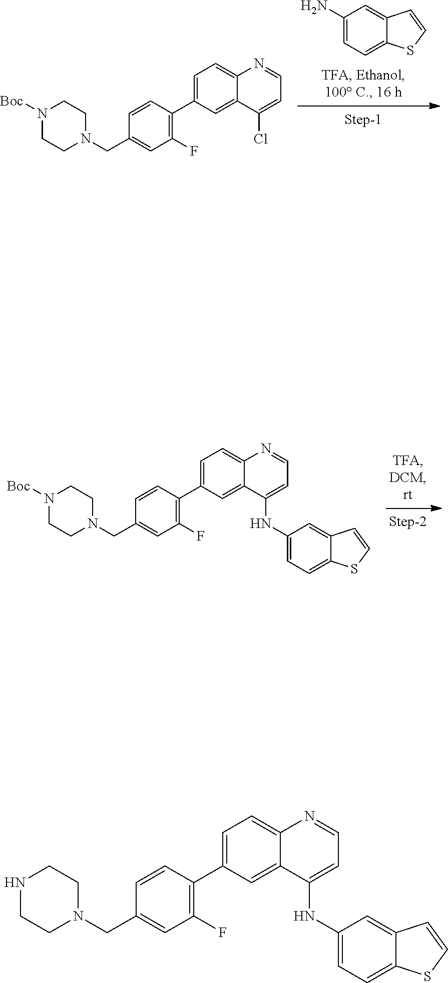 [SMILES: Nc1ccc2sccc2c1 ; CC(C)(C)OC(=O)N1CCN(Cc2ccc(-c3ccc4nccc(Cl)c4c3)c(F)c2)CC1 ; CC(C)(C)OC(=O)N1CCN(Cc2ccc(-c3ccc4nccc(Nc5ccc6sccc6c5)c4c3)c(F)c2)CC1 ; Fc1cc(CN2CCNCC2)ccc1-c1ccc2nccc(Nc3ccc4sccc4c3)c2c1]

### Step-1: Synthesis of tert-butyl 4-(4-(4-(benzo[b]thiophen-5-ylamino)quinolin-6-yl)-3-fluorobenzyl)piperazine-1-carboxylate

To a solution of tert-butyl 4-(4-(4-chloroquinolin-6-yl)-3-fluorobenzyl)piperazine-1-carboxylate (200 mg, 0.439 mmol, 1.0 equiv) and benzo[b]thiophen-5-amine (52 mg, 0.35 mmol, 0.8 equiv) in Ethanol (4 mL) was added TFA (0.2 mL) dropwise at RT. Reaction was stirred at 100° C. for 16 h. The progress of the reaction was monitored by TLC and LCMS. After completion of reaction, reaction mixture was concentrated under reduced pressure. The resulting residue was neutralized with saturated solution of NaHCO₃ (20 mL) and was extracted with DCM (3×30 mL). Combined organic layers were dried over anhydrous Na₂SO₄, filtered and concentrated under reduced. The crude product was purified by flash chromatography, elute in 2% MeOH:DCM to afford tert-butyl 4-(4-(4-(benzo[b]thiophen-5-ylamino)quinolin-6-yl)-3-fluorobenzyl)piperazine-1-carboxylate (100 mg, 40%). LCMS: 569.2 [M+1]⁺.

### Step-2: Synthesis of N-(benzo[b]thiophen-5-yl)-6-(2-fluoro-4-(piperazin-1-ylmethyl)phenyl)quinolin-4-amine

To a solution of tert-butyl 4-(4-(4-(benzo[b]thiophen-5-ylamino)quinolin-6-yl)-3-fluorobenzyl)piperazine-1-carboxylate (100 mg, 0.18 mmol, 1.0 equiv) in DCM (5 mL) was added TFA (1 mL) dropwise at 0° C. Reaction was stirred at RT for 16 h. The progress of the reaction was monitored by TLC and LCMS. After completion of reaction, reaction mixture was concentrated under reduced pressure, neutralized with saturated solution of NaHCO₃ and was extracted with DCM (3×25 mL). Combined organic layers were dried over anhydrous Na₂SO₄, filtered and concentrated under reduced pressure. The crude product was purified by flash chromatography, eluted in 10% MeOH:DCM to afford N-(benzo[b]thiophen-5-yl)-6-(2-fluoro-4-(piperazin-1-ylmethyl)phenyl)quinolin-4-amine (12 mg, 15%). LCMS: 469.2 [M+1]⁺, ¹H NMR (400 MHz, DMSO-d₆) δ 9.18 (s, 1H) 8.61 (s, 1H) 8.46 (d, J=5.09 Hz, 1H) 8.05 (d, J=8.27 Hz, 2H) 7.91-7.98 (m, 1H) 7.87 (br. s., 2H) 7.81 (d, J=5.72 Hz, 1H) 7.69 (t, J=7.95 Hz, 1H) 7.47 (d, J=5.72 Hz, 1H) 7.40 (d, J=8.27 Hz, 1H) 7.26-7.36 (m, 2H) 6.95 (d, J=5.72 Hz, 1H) 3.52-3.61 (s, 2H) 3.16 (br. s., 4H) 2.91 (br. s., 4H).

### Example 79: Synthesis of N-(benzofuran-5-yl)-6-(2-fluoro-4-(piperazin-1-ylmethyl)phenyl)quinolin-4-amine (Compound 386)

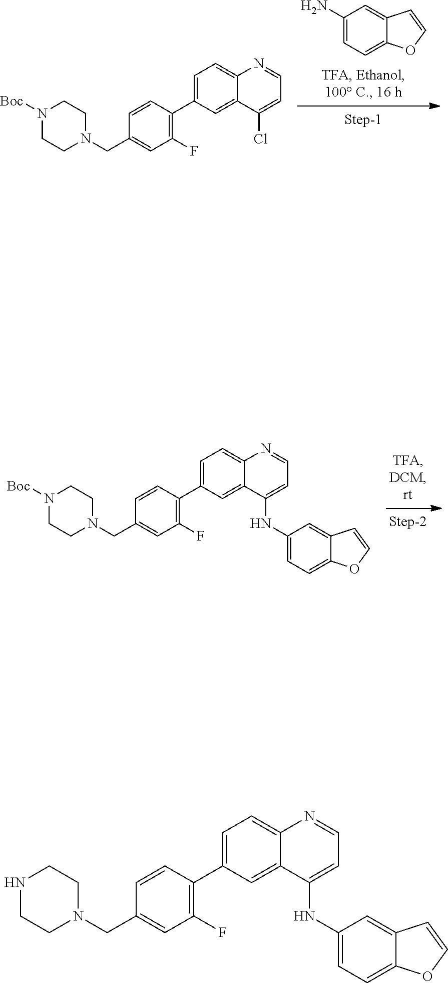 [SMILES: Nc1ccc2occc2c1 ; CC(C)(C)OC(=O)N1CCN(Cc2ccc(-c3ccc4nccc(Cl)c4c3)c(F)c2)CC1 ; CC(C)(C)OC(=O)N1CCN(Cc2ccc(-c3ccc4nccc(Nc5ccc6occc6c5)c4c3)c(F)c2)CC1 ; Fc1cc(CN2CCNCC2)ccc1-c1ccc2nccc(Nc3ccc4occc4c3)c2c1]

### Step-1: Synthesis of tert-butyl 4-(4-(4-(benzofuran-5-ylamino)quinolin-6-yl)-3-fluorobenzyl)piperazine-1-carboxylate

To a solution of tert-butyl 4-(4-(4-chloroquinolin-6-yl)-3-fluorobenzyl)piperazine-1-carboxylate carboxylate (200 mg, 0.44 mmol, 1.0 equiv) and benzofuran-5-amine (47 mg, 0.35 mmol, 0.8 equiv) in ethanol (4 mL) was added TFA (0.2 mL) dropwise at RT. Reaction was stirred at 100° C. for 16 h. The progress of the reaction was monitored by TLC and LCMS. After completion of reaction, reaction mixture was concentrated under reduced pressure. The resulting residue was neutralized with saturated solution of NaHCO₃ (20 mL) and was extracted with DCM (3×30 mL). Combined organic layers were dried over anhydrous Na₂SO₄, filtered and concentrated under reduced. The crude product was purified by flash chromatography, elute in 2% MeOH:DCM to afford tert-butyl 4-(4-(4-(benzofuran-5-ylamino)quinolin-6-yl)-3-fluorobenzyl)piperazine-1-carboxylate (80 mg, 33%). LCMS: 553.2 [M+1]⁺.

### Step-2: Synthesis of N-(benzofuran-5-yl)-6-(2-fluoro-4-(piperazin-1-ylmethyl)phenyl)quinolin-4-amine

To a solution of tert-butyl 4-(4-(4-(benzofuran-5-ylamino)quinolin-6-yl)-3-fluorobenzyl)piperazine-1-carboxylate (80 mg, 0.14 mmol, 1.0 equiv) in DCM (5 mL) was added TFA (1 mL) dropwise at 0° C. Reaction was stirred at RT for 16 h. The progress of the reaction was monitored by TLC and LCMS. After completion of reaction, reaction mixture was concentrated under reduced pressure, neutralized with saturated solution of NaHCO₃ and was extracted with DCM (3×25 mL). Combined organic layers were dried over anhydrous Na₂SO₄, filtered and concentrated under reduced pressure. The crude product was purified by flash chromatography, eluted in 10% MeOH:DCM to afford N-(benzofuran-5-yl)-6-(2-fluoro-4-(piperazin-1-ylmethyl)phenyl)quinolin-4-amine (12 mg, 15%). LCMS: 453.2 [M+1]⁺, ¹H NMR (400 MHz, DMSO-d₆) δ 9.18 (s, 1H) 8.62 (s, 1H) 8.42 (br. s., 1H) 8.04 (s, 1H) 7.91-7.98 (m, 1H) 7.88 (br. s., 1H) 7.70-7.75 (m, 1H) 7.68 (d, J=8.90 Hz, 2H) 7.63 (s, 1H) 7.25-7.34 (m, 3H) 6.99 (s, 1H) 6.78 (d, J=5.09 Hz, 1H) 3.64 (s, 2H) 3.11 (br. s., 4H) 2.67 (br. s., 4H).

### Example 80: Synthesis of 6-(2-fluoro-4-(piperazin-1-ylmethyl)phenyl)-N-(1H-indazol-5-yl)quinolin-4-amine (Compound 387)

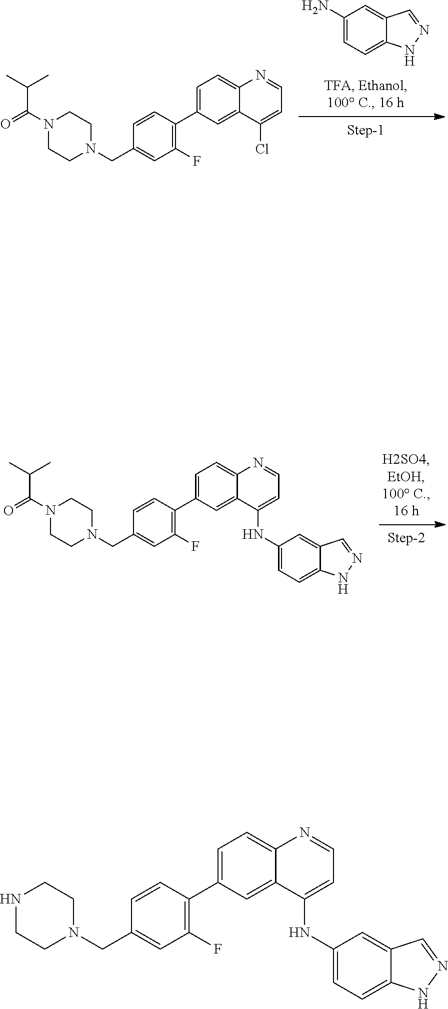 [SMILES: Nc1ccc2[nH]ncc2c1 ; CC(C)C(=O)N1CCN(Cc2ccc(-c3ccc4nccc(Cl)c4c3)c(F)c2)CC1 ; CC(C)C(=O)N1CCN(Cc2ccc(-c3ccc4nccc(Nc5ccc6[nH]ncc6c5)c4c3)c(F)c2)CC1 ; Fc1cc(CN2CCNCC2)ccc1-c1ccc2nccc(Nc3ccc4[nH]ncc4c3)c2c1]

### Step-1: Synthesis of 1-(4-(4-(4-((1H-indazol-5-yl)amino)quinolin-6-yl)-3-fluorobenzyl)piperazin-1-yl)-2-methylpropan-1-one

To a solution of 1-(4-(4-(4-chloroquinolin-6-yl)-3-fluorobenzyl)piperazin-1-yl)-2-methylpropan-1-one (200 mg, 0.47 mmol, 1.0 equiv) and 1H-indazol-5-amine (50 mg, 0.38 mmol, 0.8 equiv) in ethanol (4 mL) was added TFA (0.2 mL) dropwise at RT. Reaction was stirred at 100° C. for 16 h. The progress of the reaction was monitored by TLC and LCMS. After completion of reaction, reaction mixture was concentrated under reduced pressure. The resulting residue was neutralized with saturated solution of NaHCO₃ (20 mL) and was extracted with DCM (3×30 mL). Combined organic layers were dried over anhydrous Na₂SO₄, filtered and concentrated under reduced. The crude product was purified by flash chromatography, elute in 5% MeOH:DCM to afford 1-(4-(4-(4-((1H-indazol-5-yl)amino)quinolin-6-yl)-3-fluorobenzyl)piperazin-1-yl)-2-methylpropan-1-one (140 mg, 57%). LCMS: 523.2 [M+1]⁺.

### Step-2: Synthesis of 6-(2-fluoro-4-(piperazin-1-ylmethyl)phenyl)-N-(1H-indazol-5-yl)quinolin-4-amine

To a solution of 1-(4-(4-(4-((1H-indazol-5-yl)amino)quinolin-6-yl)-3-fluorobenzyl)piperazin-1-yl)-2-methylpropan-1-one (140 mg, 0.29 mmol, 1.0 equiv) in ethanol (5 mL) was added 20% H2SO4 (1 mL) dropwise at RT. Reaction mixture was heated at 100° C. 16 h. The progress of the reaction was monitored by TLC and LCMS. After completion of reaction, reaction mixture was concentrated under reduced pressure, basified with 2 N NaOH and was extracted with DCM (3×50 mL). Combined organic layers were dried over anhydrous Na₂SO₄, filtered and concentrated under reduced pressure. The crude product was washed with diethyl ether and dried under vacuum to afford 6-(2-fluoro-4-(piperazin-1-ylmethyl)phenyl)-N-(1H-indazol-5-yl)quinolin-4-amine (25 mg, 22%). LCMS: 453.1 [M+1]⁺, ¹H NMR (400 MHz, DMSO-d₆) δ 13.12 (br. s., 1H) 9.11 (s, 1H) 8.62 (s, 1H) 8.40 (d, J=5.72 Hz, 1H) 8.08 (s, 1H) 7.92 (d, J=8.90 Hz, 1H) 7.86 (d, J=8.90 Hz, 1H) 7.67-7.72 (m, 2H) 7.64-7.67 (m, 1H) 7.62 (s, 1H) 7.37 (d, J=8.90 Hz, 1H) 7.25-7.34 (m, 2H) 6.65-6.76 (m, 1H) 3.54 (s, 2H) 2.79 (br.s, 4H) 2.39 (br. s., 4H).

### Example 81: Synthesis of 6-(2-fluoro-4-(piperazin-1-ylmethyl)phenyl)-N-(1-methyl-1H-indazol-5-yl)quinolin-4-amine (Compound 388)

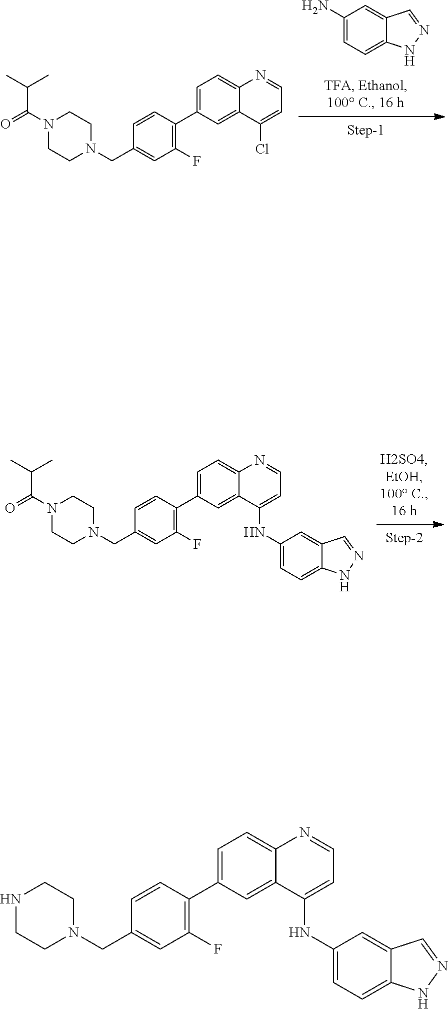 [SMILES: Nc1ccc2[nH]ncc2c1 ; CC(C)C(=O)N1CCN(Cc2ccc(-c3ccc4nccc(Cl)c4c3)c(F)c2)CC1 ; CC(C)C(=O)N1CCN(Cc2ccc(-c3ccc4nccc(Nc5ccc6[nH]ncc6c5)c4c3)c(F)c2)CC1 ; Fc1cc(CN2CCNCC2)ccc1-c1ccc2nccc(Nc3ccc4[nH]ncc4c3)c2c1]

### Step-1: Synthesis of 1-(4-(3-fluoro-4-(4-((1-methyl-1H-indazol-5-yl)amino)quinolin-6-yl)benzyl)piperazin-1-yl)-2-methylpropan-1-one

To a solution of 1-(4-(4-(4-chloroquinolin-6-yl)-3-fluorobenzyl)piperazin-1-yl)-2-methylpropan-1-one (200 mg, 0.47 mmol, 1.0 equiv) and 1-methyl-1H-indazol-5-amine (55 mg, 0.38 mmol, 0.8 equiv) in ethanol (4 mL) was added TFA (0.2 mL) dropwise at RT. Reaction was stirred at 100° C. for 16 h. The progress of the reaction was monitored by TLC and LCMS. After completion of reaction, reaction mixture was concentrated under reduced pressure. The resulting residue was neutralized with saturated solution of NaHCO₃ (20 mL) and was extracted with DCM (3×30 mL). Combined organic layers were dried over anhydrous Na₂SO₄, filtered and concentrated under reduced. The crude product was purified by flash chromatography, elute in 5% MeOH:DCM to afford 1-(4-(3-fluoro-4-(4-((1-methyl-1H-indazol-5-yl)amino)quinolin-6-yl)benzyl)piperazin-1-yl)-2-methylpropan-1-one (135 mg, 54%). LCMS: 537.3 [M+1]⁺.

### Step-2: Synthesis of 6-(2-fluoro-4-(piperazin-1-ylmethyl)phenyl)-N-(1-methyl-1H-indazol-5-yl)quinolin-4-amine

To a solution of 1-(4-(3-fluoro-4-(4-((1-methyl-1H-indazol-5-yl)amino)quinolin-6-yl)benzyl)piperazin-1-yl)-2-methylpropan-1-one (130 mg, 0.25 mmol, 1.0 equiv) in ethanol (5 mL) was added 20% H2SO4 (1 mL) dropwise at RT. Reaction mixture was heated at 100° C. 16 h. The progress of the reaction was monitored by TLC and LCMS. After completion of reaction, reaction mixture was concentrated under reduced pressure, basified with 2 N NaOH and was extracted with DCM (3×50 mL). Combined organic layers were dried over anhydrous Na₂SO₄, filtered and concentrated under reduced pressure. The crude product was washed with diethyl ether and dried under vacuum to afford 6-(2-fluoro-4-(piperazin-1-ylmethyl)phenyl)-N-(1-methyl-1H-indazol-5-yl)quinolin-4-amine (45 mg, 38%). LCMS: 467.1 [M+1]⁺, ¹H NMR (400 MHz, DMSO-d₆) δ 9.12 (s, 1H) 8.61 (s, 1H) 8.40 (d, J=5.09 Hz, 1H) 8.04 (s, 1H) 7.92 (d, J=8.90 Hz, 2H) 7.86 (d, J=8.90 Hz, 1H) 7.62-7.75 (m, 3H) 7.41 (d, J=8.27 Hz, 1H) 7.23-7.33 (m, 2H) 6.71 (d, J=5.09 Hz, 1H) 4.08 (s, 3H) 3.52 (s, 2H) 3.38 (br. s., 4H) 2.65 (br. s., 4H).

### Example 82: Synthesis of 1-(4-(4-(4-(benzo[d]thiazol-5-ylamino)quinolin-6-yl)-5-fluoro-2-methylbenzoyl)piperazin-1-yl)-2-methylpropan-1-one (Compound 282)

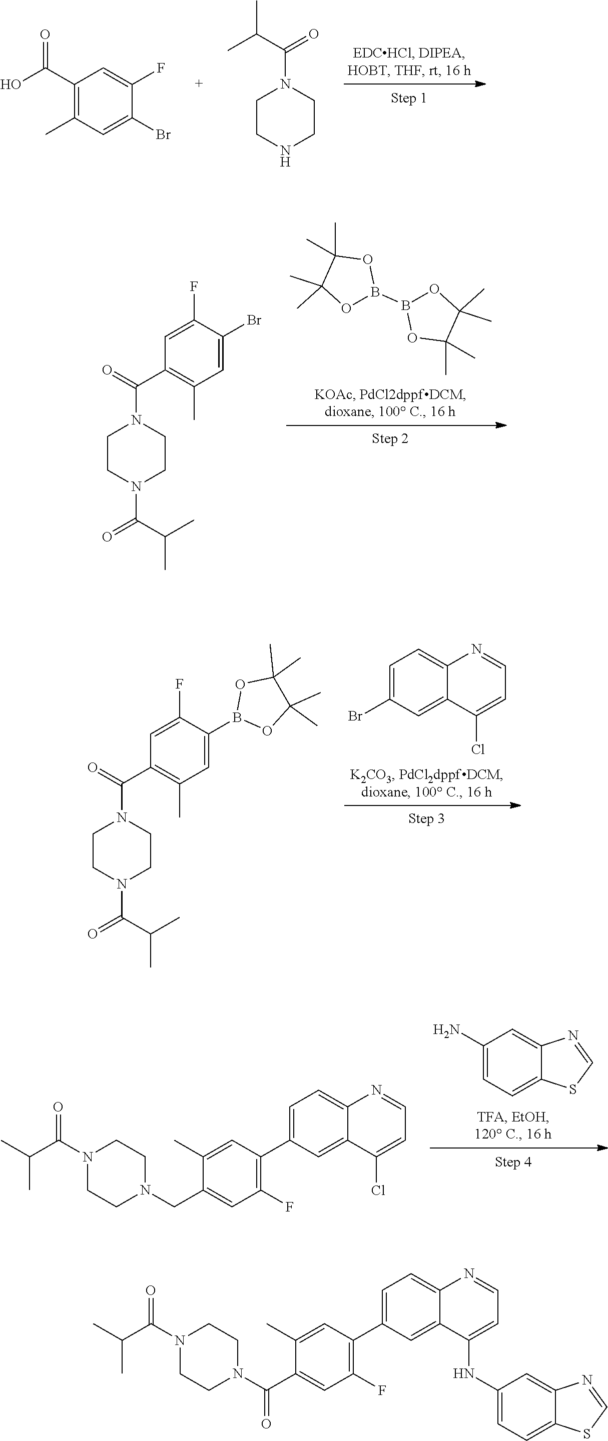

### Step-1: Synthesis of 1-(4-(4-bromo-5-fluoro-2-methylbenzoyl)piperazin-1-yl)-2-methylpropan-1-one

To a solution of 4-bromo-5-fluoro-2-methylbenzoic acid (400 mg, 1.71 mmol, 1 equiv) in THF (5 mL) was added EDC·HCl (427 mg, 2.23 mmol, 1.30 equiv) and HOBT (301 mg, 2.23 mmol, 1.30 equiv). Reaction mixture was stirred at RT for 1 h. Then added, DIPEA (0.9 mL, 5.15 mmol, 3.0 equiv) and 2-methyl-1-(piperazin-1-yl)propan-1-one (321 mg, 2.06 mmol, 1.2 equiv). Reaction was stirred at RT for 16 h. The progress of reaction was monitored by TLC. After completion of reaction, reaction mixture was diluted with water (40 mL) and extracted with ethyl acetate (2×80 mL), washed with brine solution, dried over anhydrous sodium sulphate, filtered and concentrated under reduced pressure. The crude product was purified by flash chromatography (0 to 10% Ethyl acetate in hexane as an eluent) to afford 1-(4-(4-bromo-5-fluoro-2-methylbenzoyl)piperazin-1-yl)-2-methylpropan-1-one (625 mg, 98%), LCMS: 371.1[M+1]⁺.

### Step-2: Synthesis of 1-(4-(5-fluoro-2-methyl-4-(4,4,5,5-tetramethyl-1,3,2-dioxaborolan-2-yl)benzoyl)piperazin-1-yl)-2-methylpropan-1-one

To a solution 1-(4-(4-bromo-5-fluoro-2-methylbenzoyl)piperazin-1-yl)-2-methylpropan-1-one (625 mg, 1.70 mmol, 1.0 equiv) in 1,4-dioxane (15 mL) was added KOAc (495 mg, 5.05 mmol, 3.0 equiv) and 4,4,4′,4′,5,5,5′,5′-octamethyl-2,2′-bi(1,3,2-dioxaborolane) (511 mg, 2.02 mmol, 1.2 equiv). Reaction mixture was purged with nitrogen for 5 min, then was added PdCl₂dppf·DCM (68 mg, 0.08 mmol, 0.05 equiv). The reaction was heated at 100° C. for 16 h. The progress of reaction was monitored by TLC. After completion of reaction, reaction mixture was diluted with water (50 mL) and extracted with ethyl acetate (2×100 mL), dried over anhydrous sodium sulphate, filtered and concentrated under reduced pressure. The crude product was purified by flash chromatography (0 to 10% Ethyl acetate in hexane as an eluent) to afford 1-(4-(5-fluoro-2-methyl-4-(4,4,5,5-tetramethyl-1,3,2-dioxaborolan-2-yl)benzoyl)piperazin-1-yl)-2-methylpropan-1-one (480 mg, 68%), LCMS: 337.3[M+1]⁺[Boronic-acid Fragment].

### Step-3: Synthesis of 1-(4-(4-(4-chloroquinolin-6-yl)-5-fluoro-2-methylbenzoyl)piperazin-1-yl)-2-methylpropan-1-one

To a solution of 1-(4-(5-fluoro-2-methyl-4-(4,4,5,5-tetramethyl-1,3,2-dioxaborolan-2-yl)benzoyl)piperazin-1-yl)-2-methylpropan-1-one (240 mg, 0.57 mmol, 1.0 equiv) in 1,4-dioxane:water (3.5 mL:1.5 mL) was added K₂CO₃ (158 mg, 1.14 mmol, 2.0 equiv) and 6-bromo-4-chloroquinoline (139 mg, 0.57 mmol, 1.0 equiv). Reaction mixture was purged with nitrogen for 5 min, then was added PdCl₂dppf·DCM (23 mg, 0.02 mmol, 0.05 equiv). The reaction mixture was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under reduced pressure. The crude product was purified by flash chromatography (0 to 25% Ethyl acetate in hexane as an eluent) to afford 1-(4-(4-(4-chloroquinolin-6-yl)-5-fluoro-2-methylbenzoyl)piperazin-1-yl)-2-methylpropan-1-one (180 mg, 69%), LCMS: 454.2[M+1]⁺.

### Step-4: Synthesis of 1-(4-(4-(4-(benzo[d]thiazol-5-ylamino)quinolin-6-yl)-5-fluoro-2-methylbenzoyl)piperazin-1-yl)-2-methylpropan-1-one

To a solution of 1-(4-(4-(4-chloroquinolin-6-yl)-5-fluoro-2-methylbenzoyl)piperazin-1-yl)-2-methylpropan-1-one (180 mg, 0.39 mmol, 1.0 equiv) in ethanol (5 mL) was added benzo[d]thiazol-5-amine (59 mg, 0.39 mmol, 1.0 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (15 mL), extracted with ethyl acetate (2×30 mL), dried over anhydrous sodium sulphate, filtered and concentrated under reduced pressure. The crude product was purified by flash chromatography (0 to 5% MeOH in DCM as an eluent) to afford 1-(4-(4-(4-(benzo[d]thiazol-5-ylamino)quinolin-6-yl)-5-fluoro-2-methylbenzoyl)piperazin-1-yl)-2-methylpropan-1-one (85 mg, 38%), LCMS: 568.2[M+1]. ¹H NMR (400 MHz, DMSO-d₆) δ 9.45 (s, 1H) 8.69 (s, 1H) 8.52 (d, J=5.72 Hz, 1H) 8.24 (d, J=8.90 Hz, 1H) 8.08 (s, 1H) 7.93-8.02 (m, 3H) 7.64 (d, J=7.63 Hz, 1H) 7.57 (d, J=10.17 Hz, 1H) 7.34 (br. s., 1H) 7.03 (d, J=5.09 Hz, 1H) 3.63 (br. s., 4H) 3.47 (br. s., 5H) 2.25-2.35 (m, 3H) 1.01 (br. s., 6H).

### Example 83: Synthesis of 1-(4-(4-(4-(benzo[d]thiazol-5-ylamino)quinolin-7-yl)-5-fluoro-2-methylbenzoyl)piperazin-1-yl)-2-methylpropan-1-one (Compound 283)

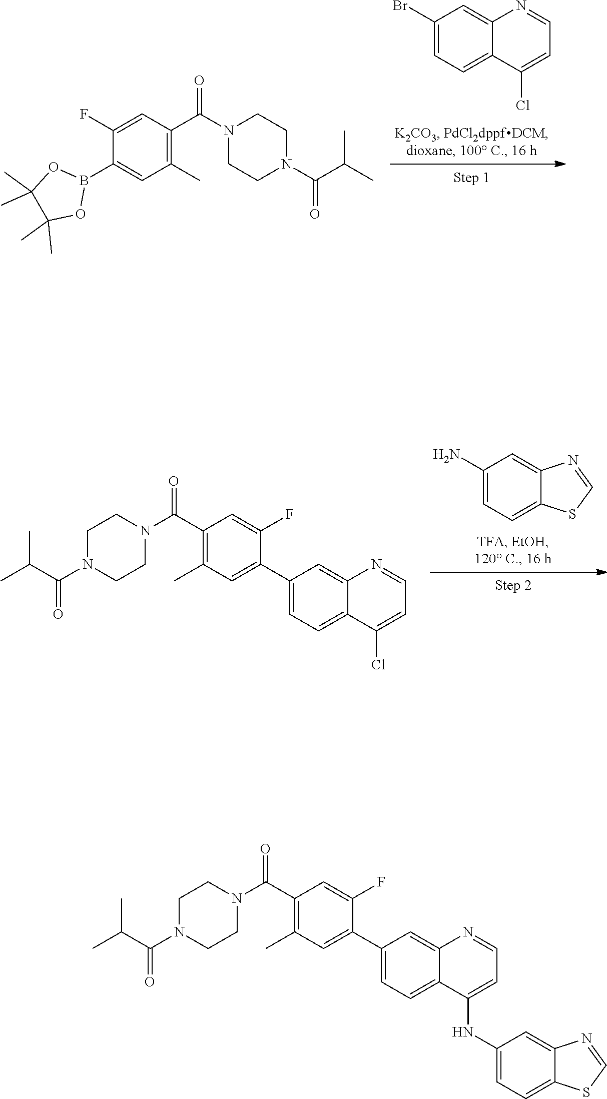

### Step-1: Synthesis of 1-(4-(4-(4-chloroquinolin-7-yl)-5-fluoro-2-methylbenzoyl)piperazin-1-yl)-2-methylpropan-1-one

To a solution of 1-(4-(5-fluoro-2-methyl-4-(4,4,5,5-tetramethyl-1,3,2-dioxaborolan-2-yl)benzoyl)piperazin-1-yl)-2-methylpropan-1-one (240 mg, 0.57 mmol, 1.0 equiv) in 1,4-dioxane:water (3.5 mL:1.5 mL) was added K₂CO₃ (158 mg, 1.14 mmol, 2.0 equiv) and 7-bromo-4-chloroquinoline (139 mg, 0.57 mmol, 1.0 equiv). Reaction mixture was purged with nitrogen for 5 min, then was added PdCl₂dppf·DCM (23 mg, 0.02 mmol, 0.05 equiv). The reaction mixture was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under reduced pressure. The crude product was purified by flash chromatography (0 to 20% Ethyl acetate in hexane as an eluent) to afford 1-(4-(4-(4-chloroquinolin-7-yl)-5-fluoro-2-methylbenzoyl)piperazin-1-yl)-2-methylpropan-1-one (200 mg, 77%), LCMS: 454.2[M+1]⁺.

### Step-2: Synthesis of 1-(4-(4-(4-(benzo[d]thiazol-5-ylamino)quinolin-7-yl)-5-fluoro-2-methylbenzoyl)piperazin-1-yl)-2-methylpropan-1-one

To a solution of 1-(4-(4-(4-chloroquinolin-7-yl)-5-fluoro-2-methylbenzoyl)piperazin-1-yl)-2-methylpropan-1-one (200 mg, 0.44 mmol, 1.0 equiv) in ethanol (5 mL) was added benzo[d]thiazol-5-amine (66 mg, 0.44 mmol, 1.0 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (15 mL), extracted with ethyl acetate (2×30 mL), dried over anhydrous sodium sulphate, filtered and concentrated under reduced pressure. The crude product was purified by flash chromatography (0 to 5% MeOH in DCM as an eluent) to afford 1-(4-(4-(4-(benzo[d]thiazol-5-ylamino)quinolin-7-yl)-5-fluoro-2-methylbenzoyl)piperazin-1-yl)-2-methylpropan-1-one (90 mg, 36%), LCMS: 568.2[M+1].

¹H NMR (400 MHz, DMSO-d₆) δ 9.45 (s, 1H) 8.69 (s, 1H) 8.52 (d, J=5.72 Hz, 1H) 8.24 (d, J=8.90 Hz, 1H) 8.08 (s, 1H) 7.93-8.02 (m, 3H) 7.64 (d, J=7.63 Hz, 1H) 7.57 (d, J=10.17 Hz, 1H) 7.34 (br. s., 1H) 7.03 (d, J=5.09 Hz, 1H) 3.63 (br. s., 4H) 3.47 (br. s., 5H) 2.25-2.35 (m, 3H) 1.01 (br. s., 6H).

### Example 84: Synthesis of (4-(4-(benzo[d]thiazol-5-ylamino)quinolin-7-yl)-3-fluorophenyl)(2,6-diazaspiro[3.3]heptan-2-yl)methanone (Example 284)

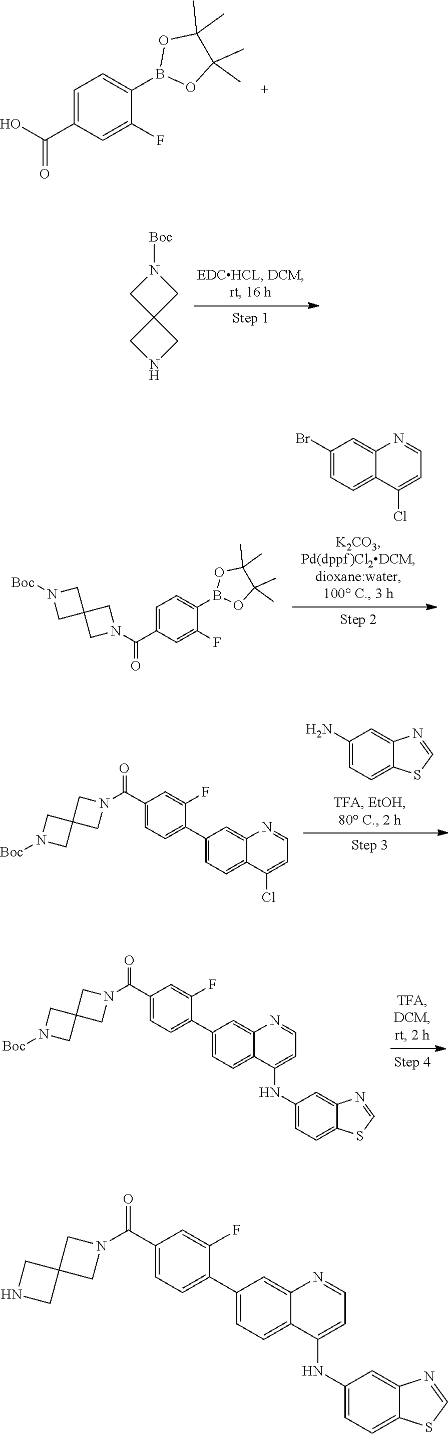

### Step-1: Synthesis of tert-butyl 6-(3-fluoro-4-(4,4,5,5-tetramethyl-1,3,2-dioxaborolan-2-yl)benzoyl)-2,6-diazaspiro[3.3]heptane-2-carboxylate

To a solution 3-fluoro-4-(4,4,5,5-tetramethyl-1,3,2-dioxaborolan-2-yl)benzoic acid (1.0 g, 3.75 mmol, 1.0 equiv) in DCM (15 mL) was added EDC·HCl (936 mg, 4.88 mmol, 1.3 equiv) and tert-butyl 2,6-diazaspiro[3.3]heptane-2-carboxylate (892 mg, 4.50 mmol, 1.0 equiv). Reaction mixture was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under vacuum to afford crude which was purified by flash chromatography (10% Ethyl acetate in hexane as an eluent) to afford tert-butyl 6-(3-fluoro-4-(4,4,5,5-tetramethyl-1,3,2-dioxaborolan-2-yl)benzoyl)-2,6-diazaspiro[3.3]heptane-2-carboxylate (1.0 g, 60%), LCMS: 365.3[M+1]⁺[boronic acid fragment].

### Step-2: Synthesis of tert-butyl 6-(4-(4-chloroquinolin-7-yl)-3-fluorobenzoyl)-2,6-diazaspiro[3.3]heptane-2-carboxylate

To a solution of tert-butyl 6-(3-fluoro-4-(4,4,5,5-tetramethyl-1,3,2-dioxaborolan-2-yl)benzoyl)-2,6-diazaspiro[3.3]heptane-2-carboxylate (1.0 g, 2.24 mmol, 1.0 equiv) in 1,4-dioxane:water (8.5 ml:1.5 ml) was added K₂CO₃ (773 mg, 5.60 mmol, 2.5 equiv) and 7-bromo-4-chloroquinoline (652 mg, 2.68 mmol, 1.2 equiv). Reaction mixture was purged with nitrogen for 5 min, then was added PdCl₂dppf·DCM (91.4 mg, 0.11 mmol, 0.05 equiv). The reaction was heated at 100° C. for 3 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography (0 to 20% Ethyl acetate in hexane as an eluent) to afford tert-butyl 6-(4-(4-chloroquinolin-7-yl)-3-fluorobenzoyl)-2,6-diazaspiro[3.3]heptane-2-carboxylate (600 mg, 60%), LCMS: 482.1 M+1]⁺.

### Step-3: Synthesis of tert-butyl 6-(4-(4-(benzo[d]thiazol-5-ylamino)quinolin-7-yl)-3-fluorobenzoyl)-2,6-diazaspiro[3.3]heptane-2-carboxylate

To a solution of tert-butyl 6-(4-(4-chloroquinolin-7-yl)-3-fluorobenzoyl)-2,6-diazaspiro[3.3]heptane-2-carboxylate (400 mg, 0.830 mmol, 1.0 equiv) in ethanol (10 mL) was added benzo[d]thiazol-5-amine (231 mg, 1.24 mmol, 1.0 equiv) and TFA (0.4 mL). The reaction mixture was heated at 80° C. for 2 h. After completion of reaction, reaction mixture was concentrated under vacuum to obtain crude which was neutralized by saturated sodium bicarbonate solution (20 mL), was extracted with ethyl acetate (2×40 mL), dried over anhydrous sodium sulphate, filtered and concentrated under vacuum to afford crude which was purified by flash chromatography (0-3% MeOH in DCM as an eluent) to afford tert-butyl 6-(4-(4-(benzo[d]thiazol-5-ylamino)quinolin-7-yl)-3-fluorobenzoyl)-2,6-diazaspiro[3.3]heptane-2-carboxylate (200 mg, 40%), LCMS: 596.1[M+1]⁺.

### Step-4: Synthesis of (4-(4-(benzo[d]thiazol-5-ylamino)quinolin-7-yl)-3-fluorophenyl)(2,6-diazaspiro[3.3]heptan-2-yl)methanone

To a solution tert-butyl 6-(4-(4-(benzo[d]thiazol-4-ylamino)quinolin-7-yl)-3-fluorobenzoyl)-2,6-diazaspiro[3.3]heptane-2-carboxylate (200 mg, 0.335 mmol, 1.0 equiv)) in dichloromethane (10 mL) was added TFA (1.5 mL) at 0° C. The reaction mixture was stirred at RT for 2 h. After completion of reaction, reaction mixture was concentrated under reduced pressure. The resulting residue was basified with saturated sodium bicarbonate solution (10 mL), was extracted with DCM (2×20 mL), dried over anhydrous sodium sulphate, filtered and concentrated under vacuum to afford crude which was purified by flash chromatography (0-8% MeOH in DCM as an eluent) to afford (4-(4-(benzo[d]thiazol-5-ylamino)quinolin-7-yl)-3-fluorophenyl)(2,6-diazaspiro[3.3]heptan-2-yl)methanone (80 mg, 48%), LCMS: 496.2[M+1]^{+ 1}H NMR (400 MHz, DMSO-d₆) δ 9.43 (s, 1H) 9.29 (s, 1H) 8.54 (d, J=5.72 Hz, 2H) 8.21 (d, J=8.27 Hz, 1H) 8.10 (s, 1H) 8.05 (s, 1H) 7.80 (t, J=7.95 Hz, 2H) 7.48-7.68 (m, 3H) 7.05 (d, J=5.09 Hz, 1H) 4.46-4.67 (m, 2H) 4.06-4.23 (m, 2H) 3.96 (br. s., 1H) 3.62 (br. s., 3H).

### Example 85: Synthesis of (4-(4-(benzo[d]thiazol-5-ylamino)quinolin-6-yl)-3-fluorophenyl)(2,6-diazaspiro[3.3]heptan-2-yl)methanone Compound 285)

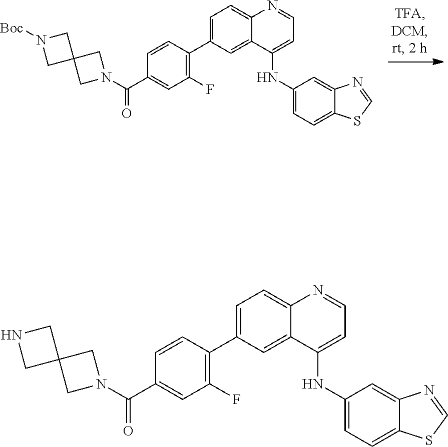 [SMILES: CC(C)(C)OC(=O)N1CC2(C1)CN(C(=O)c1ccc(-c3ccc4nccc(Nc5ccc6scnc6c5)c4c3)c(F)c1)C2 ; O=C(c1ccc(-c2ccc3nccc(Nc4ccc5scnc5c4)c3c2)c(F)c1)N1CC2(CNC2)C1]

### Synthesis of (4-(4-(benzo[d]thiazol-5-ylamino)quinolin-6-yl)-3-fluorophenyl)(2,6-diazaspiro[3.3]heptan-2-yl)methanone

To a solution tert-butyl 6-(4-(4-(benzo[d]thiazol-5-ylamino)quinolin-6-yl)-3-fluorobenzoyl)-2,6-diazaspiro[3.3]heptane-2-carboxylate (50 mg, 0.08 mmol, 1.0 equiv)) in dichloromethane (5 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (10 mL), was extracted with DCM (2×20 mL), dried over anhydrous sodium sulphate, filtered and concentrated under vacuum to afford crude which was purified by reversed phase HPLC to afford (4-(4-(benzo[d]thiazol-5-ylamino)quinolin-6-yl)-3-fluorophenyl)(2,6-diazaspiro[3.3]heptan-2-yl)methanone (12 mg, as a TFA salt), LCMS: 496.2[M+1]^{+ 1}H NMR (400 MHz, DMSO-d₆) δ 9.43 (s, 1H) 9.29 (s, 1H) 8.54 (d, J=5.72 Hz, 2H) 8.21 (d, J=8.27 Hz, 1H) 8.10 (s, 1H) 8.05 (s, 1H) 7.80 (t, J=7.95 Hz, 2H) 7.48-7.68 (m, 3H) 7.05 (d, J=5.09 Hz, 1H) 4.46-4.67 (m, 2H) 4.06-4.23 (m, 2H) 3.96 (br. s., 1H) 3.62 (br. s., 3H).

### Example 86: Synthesis of (4-(4-(benzo[d]thiazol-5-ylamino)quinolin-6-yl)-3-fluorophenyl)(3-methylpiperazin-1-yl)methanone (Compound 286)

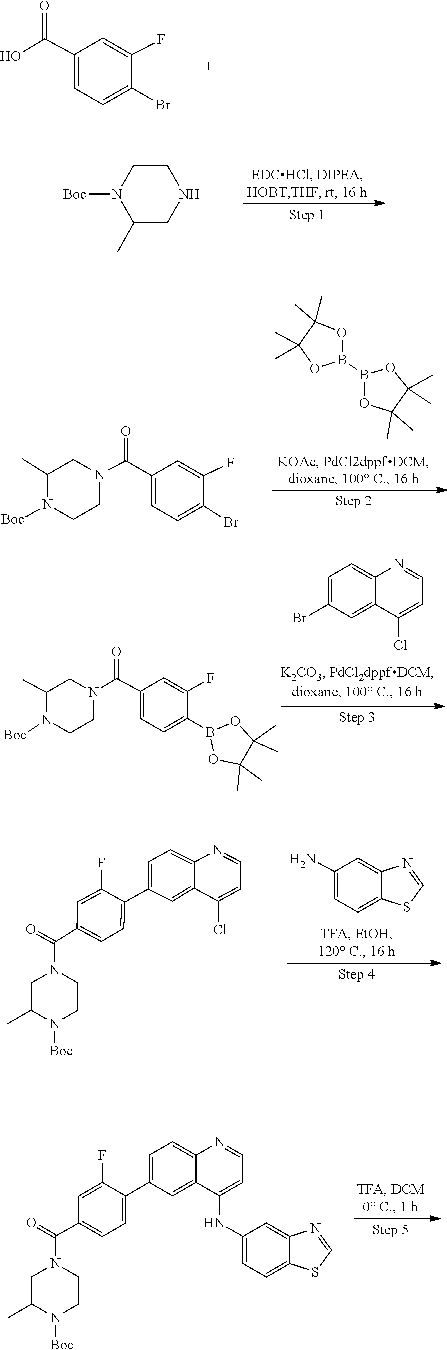

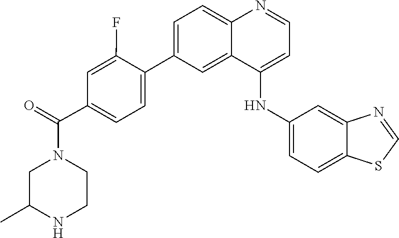 [SMILES: CC1CN(C(=O)c2ccc(-c3ccc4nccc(Nc5ccc6scnc6c5)c4c3)c(F)c2)CCN1]

### Step-1: Synthesis of tert-butyl 4-(4-bromo-3-fluorobenzoyl)-2-methylpiperazine-1-carboxylate

To a solution of 4-bromo-3-fluorobenzoic acid (200 mg, 0.91 mmol, 1.0 equiv) in THF (5 mL) was added EDC·HCl (227 mg, 1.18 mmol, 1.3 equiv) and HOBT (160 mg, 1.18 mmol, 1.3 equiv). Reaction was stirred at RT for 1 h. Then added, DIPEA (0.5 mL, 2.73 mmol, 3.0 equiv) and tert-butyl 2-methylpiperazine-1-carboxylate (219 mg, 1.09 mmol, 1.2 equiv). Reaction was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under vacuum to afford crude which was purified by flash chromatography (10% Ethyl acetate in hexane as an eluent) to afford tert-butyl 4-(4-bromo-3-fluorobenzoyl)-2-methylpiperazine-1-carboxylate (350 mg, 95%), LCMS: 401.3[M+1]⁺.

### Step-2: Synthesis of tert-butyl 4-(3-fluoro-4-(4,4,5,5-tetramethyl-1,3,2-dioxaborolan-2-yl)benzoyl)-2-methylpiperazine-1-carboxylate

To a solution tert-butyl 4-(4-bromo-3-fluorobenzoyl)-2-methylpiperazine-1-carboxylate (150 mg, 0.37 mmol, 1.0 equiv) in 1,4-dioxane (5 mL) was added potassium acetate (110 mg, 1.12 mmol, 3.0 equiv) and 4,4,4′,4′,5,5,5′,5′-octamethyl-2,2′-bi(1,3,2-dioxaborolane) (113 mg, 0.45 mmol, 1.2 equiv). Reaction mixture was purged with nitrogen for 5 min, then was added PdCl₂dppf·DCM (15 mg, 0.02 mmol, 0.05 equiv). The reaction mixture was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under vacuum to afford crude which was purified by flash chromatography (10% Ethyl acetate in hexane as an eluent) to afford tert-butyl 4-(3-fluoro-4-(4,4,5,5-tetramethyl-1,3,2-dioxaborolan-2-yl)benzoyl)-2-methylpiperazine-1-carboxylate (160 mg, 95%), LCMS: 449.3[M+1]⁺.

### Step-3: Synthesis of tert-butyl 4-(4-(4-chloroquinolin-6-yl)-3-fluorobenzoyl)-2-methylpiperazine-1-carboxylate

To a solution of tert-butyl 4-(3-fluoro-4-(4,4,5,5-tetramethyl-1,3,2-dioxaborolan-2-yl)benzoyl)-2-methylpiperazine-1-carboxylate (150 mg, 0.33 mmol, 1.0 equiv) in 1,4-dioxane:water (3.5 mL:1.5 mL) was added K₂CO₃ (92 mg, 0.67 mmol, 2.0 equiv) and 6-bromo-4-chloroquinoline (81 mg, 0.33 mmol, 1.0 equiv). Reaction mixture was purged with nitrogen for 5 min, then was added PdCl₂dppf·DCM (13 mg, 0.02 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under vacuum to afford crude which was purified by flash chromatography (10% Ethyl acetate in hexane as an eluent) to afford tert-butyl 4-(4-(4-chloroquinolin-6-yl)-3-fluorobenzoyl)-2-methylpiperazine-1-carboxylate (110 mg, 68%). LCMS: 484.1[M+2]⁺.

### Step-4: Synthesis of tert-butyl 4-(4-(4-(benzo[d]thiazol-5-ylamino)quinolin-6-yl)-3-fluorobenzoyl)-2-methylpiperazine-1-carboxylate

To a solution of tert-butyl 4-(4-(4-chloroquinolin-6-yl)-3-fluorobenzoyl)-2-methylpiperazine-1-carboxylate (110 mg, 0.23 mmol, 1.0 equiv) in ethanol (5 mL) was added benzo[d]thiazol-5-amine (51 mg, 0.34 mmol, 1.5 equiv) and TFA (0.1 mL). The reaction mixture was heated at 120° C. for 16 h. After completion of reaction, reaction mixture was concentrated under vacuum to obtain crude which was neutralized by saturated sodium bicarbonate solution (20 mL), was extracted with ethyl acetate (2×40 mL), dried over anhydrous sodium sulphate, filtered and concentrated under vacuum to afford crude which was purified by flash chromatography (0-3% MeOH in DCM as an eluent) to afford tert-butyl 4-(4-(4-(benzo[d]thiazol-5-ylamino)quinolin-6-yl)-3-fluorobenzoyl)-2-methylpiperazine-1-carboxylate (74 mg, 55%), LCMS: 598.1[M+1]⁺.

### Step-5: Synthesis of (4-(4-(benzo[d]thiazol-5-ylamino)quinolin-6-yl)-3-fluorophenyl)(3-methylpiperazin-1-yl)methanone

To a solution of tert-butyl 4-(4-(4-(benzo[d]thiazol-5-ylamino)quinolin-6-yl)-3-fluorobenzoyl)-2-methylpiperazine-1-carboxylate (74 mg, 0.12 mmol, 1.0 equiv) in DCM (5 mL) was added TFA (0.8 mL) at 0° C. The reaction mixture was stirred at RT for 1 h. After completion of reaction, reaction mixture was concentrated under reduced pressure. The resulting residue was basified with saturated sodium bicarbonate solution (10 mL), was extracted with DCM (2×20 mL), dried over anhydrous sodium sulphate, filtered and concentrated under vacuum to afford crude which was purified by flash chromatography (0-8% MeOH in DCM as an eluent) to afford (4-(4-(benzo[d]thiazol-5-ylamino)quinolin-6-yl)-3-fluorophenyl)(3-methylpiperazin-1-yl)methanone (11 mg, 18%). LCMS: 498.1[M+1]^{+ 1}H NMR (400 MHz, DMSO-d₆) δ 1.04 (d, J=6.36 Hz, 1H) 1.23 (s, 3H) 2.62-2.68 (m, 2H) 2.74-2.85 (m, 2H) 2.96-3.09 (m, 1H) 4.36 (d, J=12.08 Hz, 1H) 7.02 (d, J=5.09 Hz, 1H) 7.52-7.59 (m, 2H) 7.85-7.93 (m, 2H) 7.98 (d, J=8.27 Hz, 1H) 8.07 (s, 1H) 8.13 (d, J=8.27 Hz, 1H) 8.23 (d, J=8.27 Hz, 1H) 8.49 (d, J=5.09 Hz, 1H) 8.82 (br. s., 1H) 9.34 (s, 1H) 9.44 (s, 1H).

### Example 87: Synthesis of 4-(4-(4-(benzofuran-5-ylamino)quinolin-6-yl)-3-fluorobenzyl)piperazin-2-one. (Compound 287)

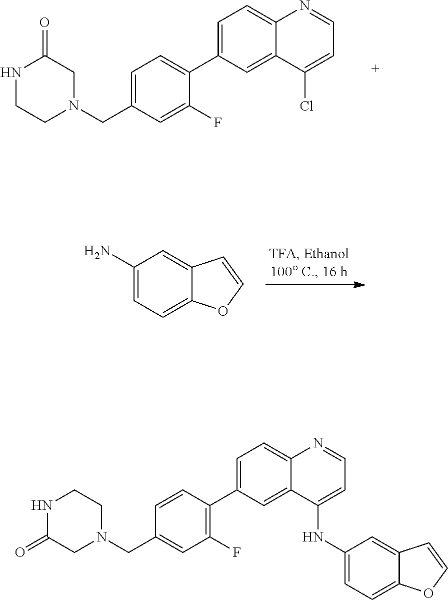 [SMILES: O=C1CN(Cc2ccc(-c3ccc4nccc(Cl)c4c3)c(F)c2)CCN1 ; Nc1ccc2occc2c1 ; O=C1CN(Cc2ccc(-c3ccc4nccc(Nc5ccc6occc6c5)c4c3)c(F)c2)CCN1]

To a solution of 4-(4-(4-chloroquinolin-6-yl)-3-fluorobenzyl)piperazin-2-one (50 mg, 0.13 mmol, 1.0 equiv) and benzofuran-5-amine (18 mg, 0.13 mmol, 1.0 equiv) in EtOH (5 mL) was added TFA (0.05 mL) and the reaction mixture was heated at 100° C. for 16 h. The progress of the reaction was monitored by TLC and LCMS. After completion of reaction, reaction mixture was concentrated under reduced pressure to obtain crude which was basified with saturated solution of NaHCO₃ (30 mL) and was extracted with DCM (2×35 mL). Combined organic layers were dried by Na₂SO₄, filtered and concentrated under reduced pressure. The crude product was purified by flash chromatography (6% MeOH in DCM as an eluent) to afford 4-(4-(4-(benzofuran-5-ylamino)quinolin-6-yl)-3-fluorobenzyl)piperazin-2-one (30 mg, 48%). LCMS: 467 [M+1]^{+ 1}H NMR (400 MHz, DMSO-d₆) δ9.29 (br. s., 1H), 8.64 (s, 1H), 8.43 (d, J=5.1 Hz, 1H), 8.05 (d, J=1.9 Hz, 1H), 7.98-7.89 (m, 2H), 7.79 (s, 1H), 7.72-7.57 (m, 3H), 7.36-7.26 (m, 3H), 7.00 (d, J=1.9 Hz, 1H), 6.78 (d, J=5.7 Hz, 1H), 3.64 (s, 2H), 3.53-3.43 (d, 2H), 2.96 (d, 2H), 2.60 (d, J=5.4 Hz, 2H).

### Example 88: Synthesis of 4-(3-fluoro-4-(4-(1-methyl-1H-indazol-5-ylamino)quinolin-6-yl)benzyl)piperazin-2-one (Compound 288)

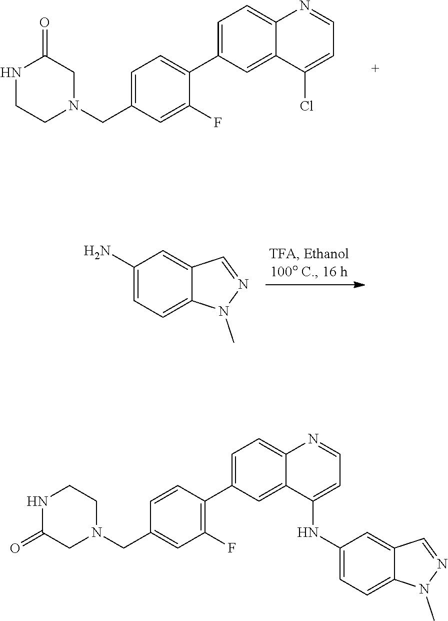 [SMILES: O=C1CN(Cc2ccc(-c3ccc4nccc(Cl)c4c3)c(F)c2)CCN1 ; Cn1ncc2cc(N)ccc21 ; Cn1ncc2cc(Nc3ccnc4ccc(-c5ccc(CN6CCNC(=O)C6)cc5F)cc34)ccc21]

To a solution of 4-(4-(4-chloroquinolin-6-yl)-3-fluorobenzyl)piperazin-2-one (50 mg, 0.13 mmol, 1.0 equiv) and 1-methyl-1H-indazol-5-amine (19 mg, 0.13 mmol, 1.0 equiv) in EtOH (5 mL) was added TFA (0.05 mL) and the reaction mixture was heated at 100° C. for 16 h. The progress of the reaction was monitored by TLC and LCMS. After completion of reaction, reaction mixture was concentrated under reduced pressure. The resulting residue was basified with saturated solution of NaHCO₃ (30 mL) and was extracted with DCM (2×35 mL). Combined organic layers were dried by Na₂SO₄, filtered and concentrated under reduced pressure. The crude product was purified by flash chromatography (6% MeOH in DCM as an eluent) to afford 4-(3-fluoro-4-(4-(1-methyl-1H-indazol-5-ylamino)quinolin-6-yl)benzyl)piperazin-2-one (30 mg, 46%). LCMS: 481 [M+1]⁺, ¹H NMR (400 MHz, DMSO-d₆) δ 8.77 (s, 1H), 8.45 (d, J=5.7 Hz, 2H), 8.19-8.04 (m, 1H), 8.04-7.93 (m, 2H), 7.85-7.70 (m, 4H), 7.44 (d, J=10.2 Hz, 1H), 7.37 (s, 1H), 7.34 (s, 1H), 6.75-6.62 (m, 1H), 4.10 (s, 3H), 3.77-3.53 (s, 2H), 3.18 (br. s., 2H), 2.96 (d, 2H), 2.61 (d, J=5.4 Hz, 2H).

### Example 89: Synthesis of 4-(4-(4-(benzo[d]isoxazol-5-ylamino)quinolin-6-yl)-3-fluorobenzyl)piperazin-2-one (Compound 289)

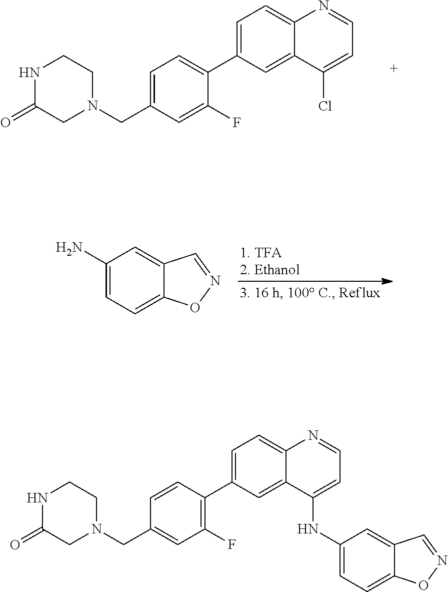 [SMILES: O=C1CN(Cc2ccc(-c3ccc4nccc(Cl)c4c3)c(F)c2)CCN1 ; Nc1ccc2oncc2c1 ; O=C1CN(Cc2ccc(-c3ccc4nccc(Nc5ccc6oncc6c5)c4c3)c(F)c2)CCN1]

To a solution of afford 4-(4-(4-chloroquinolin-6-yl)-3-fluorobenzyl)piperazin-2-one (30 mg, 0.08 mmol, 1.0 equiv) and benzo[d]isoxazol-5-amine (11 mg, 0.08 mmol, 1.0 equiv) in EtOH (3 mL) was added TFA (0.05 mL) and the reaction mixture was heated at 100° C. for 16 h. The progress of the reaction was monitored by TLC and LCMS. After completion of reaction, reaction mixture was concentrated under reduced pressure. The resulting residue was basified with saturated solution of NaHCO₃ (30 mL) and was extracted with DCM (2×35 mL). Combined organic layers were dried by Na₂SO₄, filtered and concentrated under reduced pressure. The crude product was purified by flash chromatography (6% MeOH in DCM as an eluent) to afford 4-(4-(4-(benzo[d]isoxazol-5-ylamino)quinolin-6-yl)-3-fluorobenzyl)piperazin-2-one (17 mg, 46%). LCMS: 468 [M+1]^{+ 1}, 1H NMR (400 MHz, DMSO-d₆) δ 11.60 (s, 1H), 10.88 (br. s., 1H), 8.90 (br. s., 1H), 8.54 (d, J=7.0 Hz, 1H), 8.23 (d, J=8.9 Hz, 2H), 8.12 (d, J=8.9 Hz, 1H), 7.79 (d, J=2.5 Hz, 2H), 7.60 (dd, J=2.5, 8.9 Hz, 1H), 7.40 (br. s., 2H), 7.23 (d, J=8.9 Hz, 1H), 6.78 (d, J=7.0 Hz, 1H), 3.63 (s, 2H), 3.53-3.43 (d, 2H), 2.96 (d, 2H), 2.85-2.58 (m, 2H).

### Example 90: Synthesis of N-(6-(4-((3,9-diazaspiro[5.5]undecan-3-yl)methyl)-2-fluorophenyl)quinolin-4-yl)benzo[d]thiazol-5-amine (Compound 290)

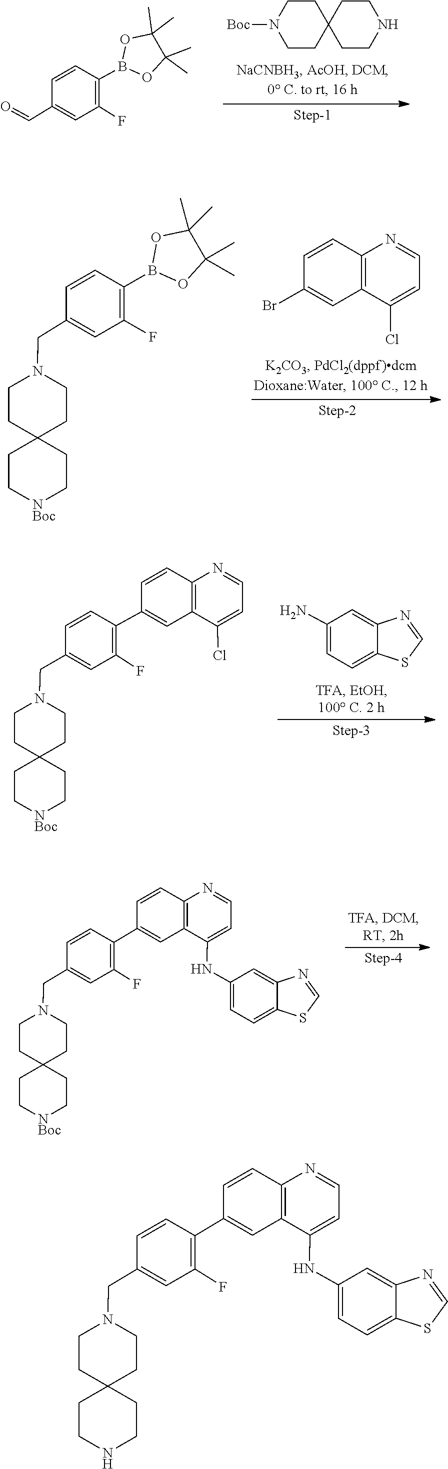

### Step-1: Synthesis of tert-butyl 9-(3-fluoro-4-(4,4,5,5-tetramethyl-1,3,2-dioxaborolan-2-yl)benzyl)-3,9-diazaspiro[5.5]undecane-3-carboxylate

To a stirred solution 3-fluoro-4-(4,4,5,5-tetramethyl-1,3,2-dioxaborolan-2-yl)benzaldehyde (200 mg, 0.8 mmol, 1.0 equiv) and tert-butyl 3,9-diazaspiro[5.5]undecane-3-carboxylate (203 mg, 0.8 mmol, 1.0 equiv) in DCM (5 mL) was added acetic acid (0.1 mL) at 0° C. Reaction mixture was stirred at RT for 1 h. NaCNBH₃ (75 mg, 1.2 mmol, 1.5 equiv) was added portion wise at 0° C. After addition, reaction mixture was stirred at RT for 16 h. The progress of reaction was monitored by LCMS. After completion of reaction, reaction mixture was diluted with water (30 mL) and was extracted with DCM (2×60 mL). The combined organic layer was washed with saturated NaHCO₃ solution (50 mL). The organic layer was dried over anhydrous sodium sulphate, filtered and concentrated under reduced pressure. The crude product was purified by flash chromatography (0 to 30% ethyl acetate in hexane as an eluent) to afford tert-butyl 9-(3-fluoro-4-(4,4,5,5-tetramethyl-1,3,2-dioxaborolan-2-yl)benzyl)-3,9-diazaspiro[5.5]undecane-3-carboxylate (130 mg, 33%), LCMS: 407.2 [Boronic-acid Fragment].

### Step-2: Synthesis of tert-butyl 9-(4-(4-chloroquinolin-6-yl)-3-fluorobenzyl)-3,9-diazaspiro[5.5]undecane-3-carboxylate

To a solution tert-butyl 9-(3-fluoro-4-(4,4,5,5-tetramethyl-1,3,2-dioxaborolan-2-yl)benzyl)-3,9-diazaspiro[5.5]undecane-3-carboxylate (130 mg, 0.27 mmol, 1.0 equiv) in 1,4-dioxane:water (8 mL:2 mL) was added K₂CO₃ (73 mg, 0.53 mmol, 2.0 equiv) and 6-bromo-4-chloroquinoline (71 mg, 0.29 mmol, 1.1 equiv). Reaction mixture was purged with nitrogen for 5 min, then was added PdCl₂dppf·DCM (10 mg, 0.013 mmol, 0.05 equiv). The reaction mixture was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure to afford crude which was purified by flash column chromatography (0 to 20% Ethyl acetate in hexane as an eluent) to afford tert-butyl 9-(4-(4-chloroquinolin-6-yl)-3-fluorobenzyl)-3,9-diazaspiro[5.5]undecane-3-carboxylate (90 mg, 65%), LCMS: 524.2 [M+1]⁺.

### Step-3: Synthesis of tert-butyl 9-(4-(4-(benzo[d]thiazol-5-ylamino)quinolin-6-yl)-3-fluorobenzyl)-3,9-diazaspiro[5.5]undecane-3-carboxylate

To a solution of tert-butyl 9-(4-(4-chloroquinolin-6-yl)-3-fluorobenzyl)-3,9-diazaspiro[5.5]undecane-3-carboxylate (90 mg, 0.17 mmol, 1.0 mmol) in ethanol (5 mL) was added benzo[d]thiazol-5-amine (25 mg, 0.09 mmol, 1.0 equiv) and TFA (0.1 mL). The reaction mixture was heated at 100° C. for 2 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 5% MeOH in DCM as an eluent) to afford tert-butyl 9-(4-(4-(benzo[d]thiazol-5-ylamino)quinolin-6-yl)-3-fluorobenzyl)-3,9-diazaspiro[5.5]undecane-3-carboxylate (50 mg, 46%), LCMS: 638.1 [M+1]⁺.

### Step-4: Synthesis of N-(6-(4-((3,9-diazaspiro[5.5]undecan-3-yl)methyl)-2-fluorophenyl)quinolin-4-yl)benzo[d]thiazol-5-amine

To a solution of tert-butyl 9-(4-(4-(benzo[d]thiazol-5-ylamino)quinolin-6-yl)-3-fluorobenzyl)-3,9-diazaspiro[5.5]undecane-3-carboxylate (50 mg, 0.08 mmol, 1.0 equiv) in DCM (5 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford N-(6-(4-((3,9-diazaspiro[5.5]undecan-3-yl)methyl)-2-fluorophenyl)quinolin-4-yl)benzo[d]thiazol-5-amine (15 mg, 35%), LCMS: 538.1[M+1]^{+ 1}H NMR (400 MHz, DMSO-d₆) δ 1.53 (br. s., 4H) 3.07 (br. s., 5H) 3.50 (br. s., 3H) 4.44 (br. s., 4H) 6.54 (s, 1H) 6.94 (d, J=6.99 Hz, 1H) 7.53 (br. s., 1H) 7.63 (d, J=8.27 Hz, 2H) 8.22 (d, J=5.09 Hz, 2H) 8.40 (d, J=8.27 Hz, 1H) 8.47 (br. s., 1H) 8.56-8.63 (m, 2H) 8.83 (d, J=9.54 Hz, 1H) 9.54 (s, 1H).

### Example 91: Synthesis of N-(7-(4-((3,9-diazaspiro[5.5]undecan-3-yl)methyl)-2-fluorophenyl)quinolin-4-yl)benzo[d]thiazol-5-amine (Compound 291)

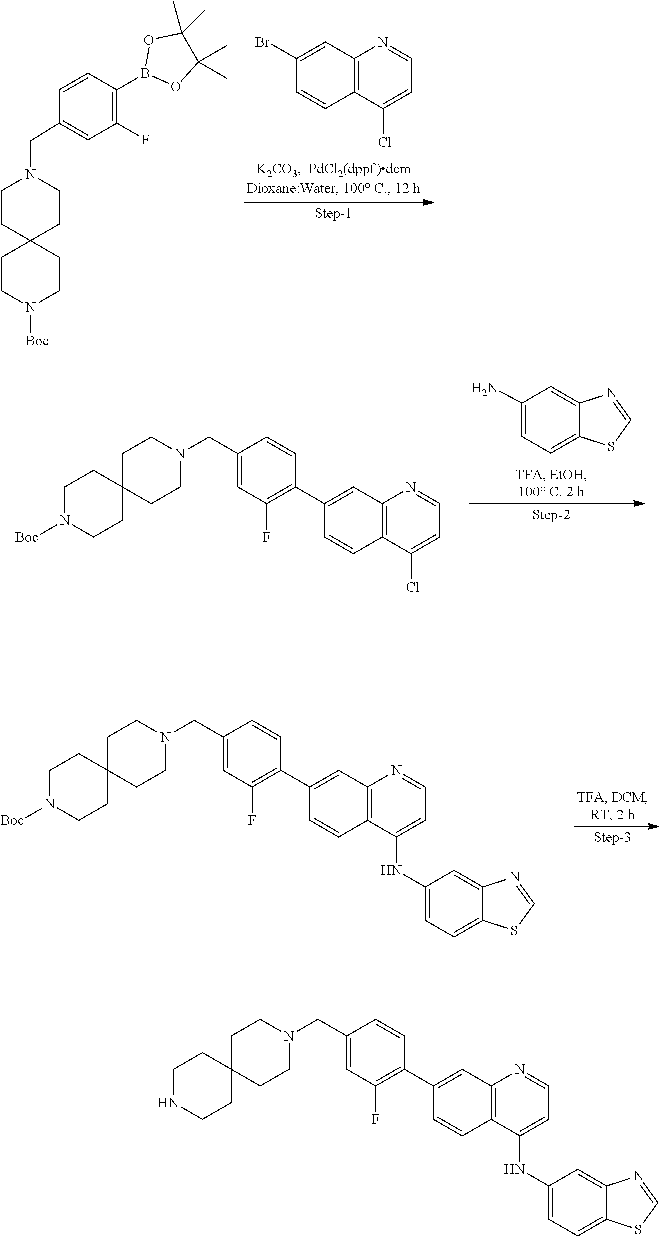

### Step-1: Synthesis of tert-butyl 9-(4-(4-chloroquinolin-7-yl)-3-fluorobenzyl)-3,9-diazaspiro[5.5]undecane-3-carboxylate

To a solution tert-butyl 9-(3-fluoro-4-(4,4,5,5-tetramethyl-1,3,2-dioxaborolan-2-yl)benzyl)-3,9-diazaspiro[5.5]undecane-3-carboxylate (130 mg, 0.27 mmol, 1.0 equiv) in 1,4-dioxane:water (8 mL:2 mL) was added K₂CO₃ (73 mg, 0.53 mmol, 2.0 equiv) and 7-bromo-4-chloroquinoline (71 mg, 0.29 mmol, 1.1 equiv). Reaction mixture was purged with nitrogen for 5 min, then was added PdCl₂dppf·DCM (10 mg, 0.013 mmol, 0.05 equiv). The reaction mixture was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure to afford crude which was purified by flash column chromatography (0 to 20% Ethyl acetate in hexane as an eluent) to afford tert-butyl 9-(4-(4-chloroquinolin-7-yl)-3-fluorobenzyl)-3,9-diazaspiro[5.5]undecane-3-carboxylate (90 mg, 64%), LCMS: 524.2 [M+1]+

### Step-2: Synthesis of tert-butyl 9-(4-(4-(benzo[d]thiazol-5-ylamino)quinolin-7-yl)-3-fluorobenzyl)-3,9-diazaspiro[5.5]undecane-3-carboxylate

To a solution of tert-butyl 9-(4-(4-chloroquinolin-7-yl)-3-fluorobenzyl)-3,9-diazaspiro[5.5]undecane-3-carboxylate (90 mg, 0.17 mmol, 1.0 mmol) in ethanol (5 mL) was added benzo[d]thiazol-5-amine (25 mg, 0.095 mmol, 1.0 equiv) and TFA (0.1 mL). The reaction mixture was heated at 100° C. for 2 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 5% MeOH in DCM as an eluent) to afford tert-butyl 9-(4-(4-(benzo[d]thiazol-5-ylamino)quinolin-7-yl)-3-fluorobenzyl)-3,9-diazaspiro[5.5]undecane-3-carboxylate (50 mg, 46%), LCMS: 638.1 [M+1]⁺.

### Step-3: Synthesis of N-(7-(4-((3,9-diazaspiro[5.5]undecan-3-yl)methyl)-2-fluorophenyl)quinolin-4-yl)benzo[d]thiazol-5-amine

To a solution of tert-butyl 9-(4-(4-(benzo[d]thiazol-5-ylamino)quinolin-7-yl)-3-fluorobenzyl)-3,9-diazaspiro[5.5]undecane-3-carboxylate (50 mg, 0.08 mmol, 1.0 equiv) in DCM (5 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford N-(7-(4-((3,9-diazaspiro[5.5]undecan-3-yl)methyl)-2-fluorophenyl)quinolin-4-yl)benzo[d]thiazol-5-amine (3.9 mg, 9%), LCMS: 538.1[M+1]^{+ 1}H NMR (400 MHz, DMSO-d₆) δ 1.53 (br. s., 4H) 3.07 (br. s., 5H) 3.50 (br. s., 3H) 4.44 (br. s., 4H) 6.54 (s, 1H) 6.94 (d, J=6.99 Hz, 1H) 7.53 (br. s., 1H) 7.63 (d, J=8.27 Hz, 2H) 8.22 (d, J=5.09 Hz, 2H) 8.40 (d, J=8.27 Hz, 1H) 8.47 (br. s., 1H) 8.56-8.63 (m, 2H) 8.83 (d, J=9.54 Hz, 1H) 9.54 (s, 1H).

### Example 92: Synthesis of N-(6-(4-((2,6-diazaspiro[3.3]heptan-2-yl)methyl)-2-fluorophenyl)quinolin-4-yl)benzo[d]thiazol-5-amine (Compound 292)

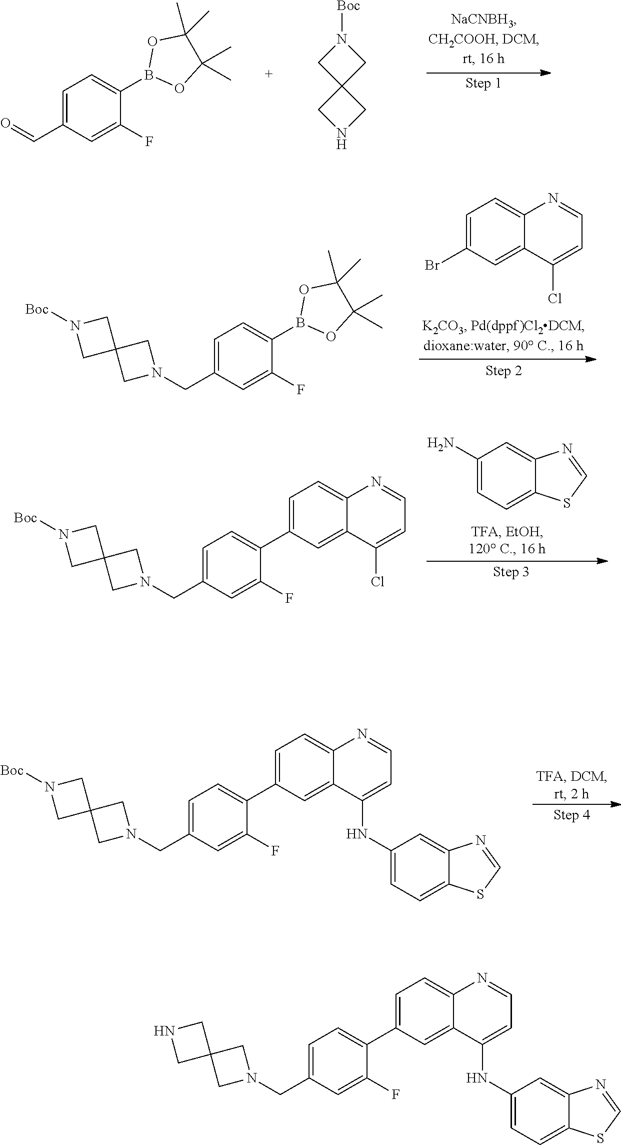

### Step-1: Synthesis of tert-butyl 6-(3-fluoro-4-(4,4,5,5-tetramethyl-1,3,2-dioxaborolan-2-yl)benzyl)-2,6-diazaspiro[3.3]heptane-2-carboxylate

To a solution of 3-fluoro-4-(4,4,5,5-tetramethyl-1,3,2-dioxaborolan-2-yl)benzaldehyde (200 mg, 0.80 mmol, 1.0 equiv) in DCM (15 mL) was added acetic acid (0.1 mL) and tert-butyl 2,6-diazaspiro[3.3]heptane-2-carboxylate (158 mg, 0.80 mmol, 1.0 equiv). Reaction mixture was stirred for 1 h, then was added NaCNBH3 (75 mg, 1.2 mmol, 1.5 equiv) at 0° C. Reaction was stirred at RT for overnight. The progress of reaction was monitored by TLC. After completion of reaction, reaction mixture was diluted with water (30 mL) and extracted with DCM (2×60 ml), washed with brine solution, dried over anhydrous sodium sulphate, filtered and concentrated under reduced pressure to afford tert-butyl 6-(3-fluoro-4-(4,4,5,5-tetramethyl-1,3,2-dioxaborolan-2-yl)benzyl)-2,6-diazaspiro[3.3]heptane-2-carboxylate [Boronic acid fragment](130 mg, 37%), LCMS: 351.2[M+1]⁺[Boronic acid fragment]

### Step-2: Synthesis of tert-butyl 6-(4-(4-chloroquinolin-6-yl)-3-fluorobenzyl)-2,6-diazaspiro[3.3]heptane-2-carboxylate

To a solution of tert-butyl 6-(3-fluoro-4-(4,4,5,5-tetramethyl-1,3,2-dioxaborolan-2-yl)benzyl)-2,6-diazaspiro[3.3]heptane-2-carboxylate (130 mg, 0.30 mmol, 1.0 equiv) in 1,4-dioxane:water (8.5 ml:1.5 ml) was added K₂CO₃ (62 mg, 0.45 mmol, 1.5 equiv) and 6-bromo-4-chloroquinoline (87 gm, 0.361 mmol, 1.2 equiv). Reaction mixture was purged with nitrogen for 5 min, then was added PdCl₂dppf·DCM (12 mg, 0.015 mmol, 0.05 equiv). The reaction was heated at 90° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure to afford crude which was purified by flash column chromatography (0 to 20% Ethyl acetate in hexane as an eluent) to afford tert-butyl 6-(4-(4-chloroquinolin-6-yl)-3-fluorobenzyl)-2,6-diazaspiro[3.3]heptane-2-carboxylate (80 mg, 57%), LCMS: 468.2 M+1]⁺.

### Step-3: Synthesis of tert-butyl 6-(4-(4-(benzo[d]thiazol-5-ylamino)quinolin-6-yl)-3-fluorobenzyl)-2,6-diazaspiro[3.3]heptane-2-carboxylate

To a solution of tert-butyl 6-(4-(4-chloroquinolin-6-yl)-3-fluorobenzyl)-2,6-diazaspiro[3.3]heptane-2-carboxylate (80 mg, 0.17 mmol, 1.0 equiv) in ethanol (10 mL) was added benzo[d]thiazol-5-amine (26 mg, 0.17 mmol, 1.0 equiv) and TFA (0.05 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 5% MeOH in DCM as an eluent) to afford tert-butyl 6-(4-(4-(benzo[d]thiazol-5-ylamino)quinolin-6-yl)-3-fluorobenzyl)-2,6-diazaspiro[3.3]heptane-2-carboxylate (80 mg, 80%), LCMS: 582.1[M+1]⁺.

### Step-4: Synthesis of N-(6-(4-((2,6-diazaspiro[3.3]heptan-2-yl)methyl)-2-fluorophenyl)quinolin-4-yl)benzo[d]thiazol-5-amine

To a solution tert-butyl 6-(4-(4-(benzo[d]thiazol-5-ylamino)quinolin-6-yl)-3-fluorobenzyl)-2,6-diazaspiro[3.3]heptane-2-carboxylate (80 mg, 0.14 mmol, 1.0 equiv)) in dichloromethane (5 mL) was added TFA (0.4 mL) at 0° C. The reaction mixture was stirred at RT for 2 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford N-(6-(4-((2,6-diazaspiro[3.3]heptan-2-yl)methyl)-2-fluorophenyl)quinolin-4-yl)benzo[d]thiazol-5-amine (40 mg, 60%), LCMS: 482.2[M+1]⁺. ¹H NMR (400 MHz, DMSO-d₆) δ 2.18 (t, J=7.31 Hz, 1H) 4.16 (br. s., 4H) 4.28 (br. s., 4H) 4.41 (br. s., 2H) 6.96 (d, J=6.99 Hz, 1H) 7.45-7.55 (m, 2H) 7.62 (d, J=8.90 Hz, 1H) 7.85 (t, J=7.63 Hz, 1H) 8.12 (d, J=8.90 Hz, 1H) 8.22-8.26 (m, 2H) 8.40 (d, J=8.27 Hz, 1H) 8.58 (d, J=6.99 Hz, 1H) 8.77 (br. s., 1H) 8.96 (br. s., 1H) 9.54 (s, 1H).

### Example 93: Synthesis of (4-(4-(benzo[d]thiazol-5-ylamino)quinolin-6-yl)-3-fluorophenyl)((3R,5S)-3,5-dimethylpiperazin-1-yl)methanone (Compound 293)

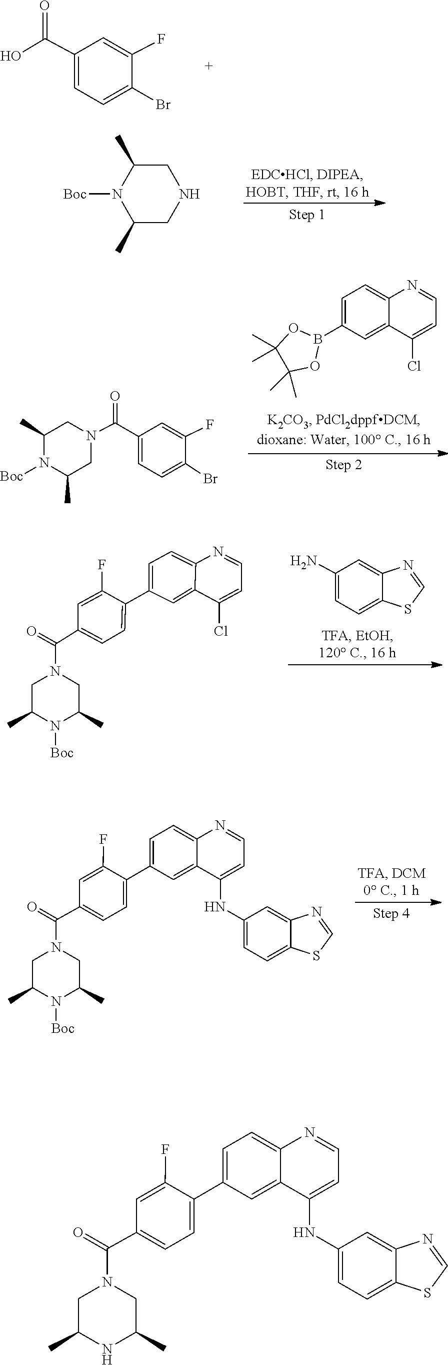

### Step-1: Synthesis of tert-butyl (2R,6S)-4-(4-bromo-3-fluorobenzoyl)-2,6-dimethylpiperazine-1-carboxylate

To a solution of 4-bromo-3-fluorobenzoic acid (200 mg, 0.91 mmol, 1.0 equiv) in THF (5 mL) was added EDC·HCl (227 mg, 1.18 mmol, 1.30 equiv) and HOBT (160 mg, 1.18 mmol, 1.3 equiv). Reaction mixture was stirred at RT for 1 h. DIPEA (0.5 mL, 2.73 mmol, 3.0 equiv) and tert-butyl (2S,6R)-2,6-dimethylpiperazine-1-carboxylate (234 mg, 1.09 mmol, 1.2 equiv). Reaction was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under reduced pressure. The crude product was purified by flash chromatography (0 to 20% Ethyl acetate in hexane as an eluent) to afford tert-butyl (2R,6S)-4-(4-bromo-3-fluorobenzoyl)-2,6-dimethylpiperazine-1-carboxylate (370 mg, 97%), LCMS: 417.1[M+2]⁺.

### Step-2: Synthesis of tert-butyl (2R,6S)-4-(4-(4-chloroquinolin-6-yl)-3-fluorobenzoyl)-2,6-dimethylpiperazine-1-carboxylate

To a solution of tert-butyl (2R,6S)-4-(4-bromo-3-fluorobenzoyl)-2,6-dimethylpiperazine-1-carboxylate (150 mg, 0.36 mmol, 1.0 equiv) in 1,4-dioxane:water (3.5 mL:1.5 mL) was added K₂CO₃ (99 mg, 0.72 mmol, 2.0 equiv) and 4-chloro-6-(4,4,5,5-tetramethyl-1,3,2-dioxaborolan-2-yl)quinoline (104 mg, 0.36 mmol, 1.0 equiv). Reaction mixture was purged with nitrogen for 5 min, then was added PdCl₂dppf·DCM (14 mg, 0.018 mmol, 0.05 equiv). The reaction mixture was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure. The crude product was purified by flash chromatography (0 to 20% Ethyl acetate in hexane as an eluent) to afford tert-butyl (2R,6S)-4-(4-(4-chloroquinolin-6-yl)-3-fluorobenzoyl)-2,6-dimethylpiperazine-1-carboxylate (128 mg, 71%), LCMS: 498.2[M+2]⁺.

### Step-3: Synthesis of tert-butyl (2R,6S)-4-(4-(4-(benzo[d]thiazol-5-ylamino)quinolin-6-yl)-3-fluorobenzoyl)-2,6-dimethylpiperazine-1-carboxylate

To a solution of tert-butyl (2R,6S)-4-(4-(4-chloroquinolin-6-yl)-3-fluorobenzoyl)-2,6-dimethylpiperazine-1-carboxylate (128 mg, 0.26 mmol, 1.0 equiv) in ethanol (5 mL) was added benzo[d]thiazol-5-amine (57 mg, 0.38 mmol, 1.5 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 5% MeOH in DCM as an eluent) to afford tert-butyl (2R,6S)-4-(4-(4-(benzo[d]thiazol-5-ylamino)quinolin-6-yl)-3-fluorobenzoyl)-2,6-dimethylpiperazine-1-carboxylate (68 mg, 43%), LCMS: 612.2[M+1]⁺.

### Step-4: Synthesis of (4-(4-(benzo[d]thiazol-5-ylamino)quinolin-6-yl)-3-fluorophenyl)((3R,5S)-3,5-dimethylpiperazin-1-yl)methanone

To a solution of tert-butyl (2R,6S)-4-(4-(4-(benzo[d]thiazol-5-ylamino)quinolin-6-yl)-3-fluorobenzoyl)-2,6-dimethylpiperazine-1-carboxylate (60 mg, 0.02 mmol, 1.0 equiv) in DCM (5 mL) was added TFA (0.3 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford crude which was purified by flash chromatography to afford (4-(4-(benzo[d]thiazol-5-ylamino)quinolin-6-yl)-3-fluorophenyl)((3R,5S)-3,5-dimethylpiperazin-1-yl)methanone (10 mg, 20%), LCMS: 512.2[M+1]^{+ 1}H NMR (400 MHz, DMSO-d₆) δ 0.89 (br. s., 3H) 1.04 (br. s., 3H) 2.67 (br. s., 3H) 2.72 (br. s., 2H) 7.05 (d, J=5.09 Hz, 1H) 7.37-7.44 (m, 2H) 7.55 (d, J=6.99 Hz, 1H) 7.79-7.83 (m, 1H) 7.92-7.98 (m, 1H) 8.04 (s, 1H) 8.16 (s, 2H) 8.22 (s, 1H) 8.52 (d, J=5.72 Hz, 1H) 8.66 (s, 1H) 9.43 (s, 1H).

### Example 94: Synthesis of (6-(4-(benzo[d]thiazol-5-ylamino)quinolin-6-yl)pyridin-3-yl)(2,6-diazaspiro[3.3]heptan-2-yl)methanone (Compound 294)

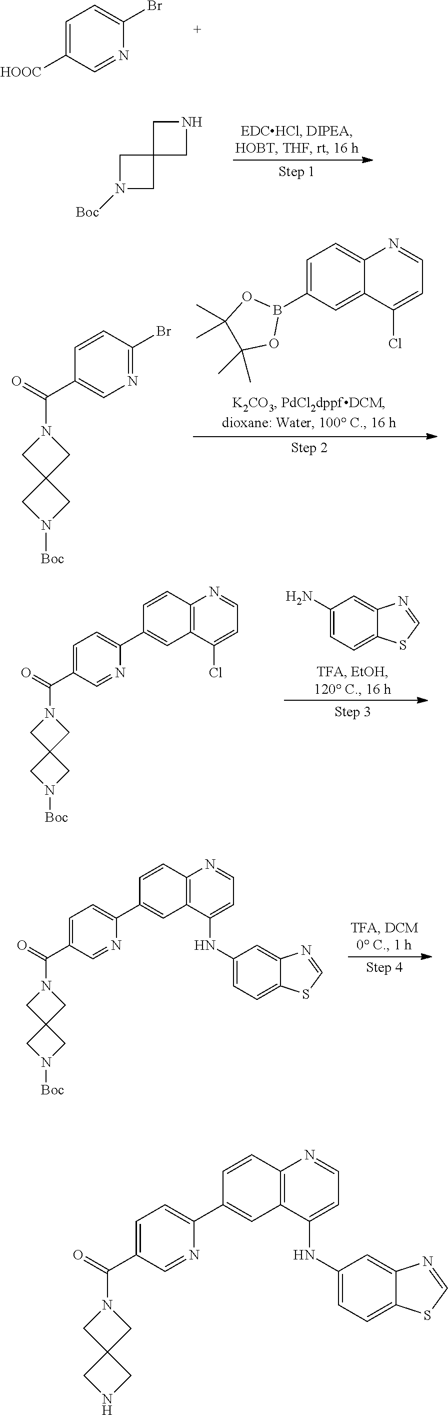

### Step-1: Synthesis of tert-butyl 6-(6-bromonicotinoyl)-2,6-diazaspiro[3.3]heptane-2-carboxylate

To a solution of 6-bromonicotinic acid (400 mg, 1.98 mmol, 1.0 equiv) in THF (10 mL) was added EDC·HCl (493 mg, 2.57 mmol, 1.3 equiv) and HOBT (347 mg, 2.57 mmol, 1.3 equiv). Reaction was stirred at RT for 1 h. DIPEA (1 mL, 5.940 mmol, 3 equiv) and tert-butyl 2,6-diazaspiro[3.3]heptane-2-carboxylate (471 mg, 2.38 mmol, 1.2 equiv). Reaction was stirred at RT for 16 h. The progress of reaction was monitored by TLC. After completion of reaction, reaction mixture was diluted with water (30 mL) and extracted with ethyl acetate (2×60 mL), washed with brine solution, dried over anhydrous sodium sulphate, filtered and concentrated under reduced pressure. The crude product was purified by flash chromatography (0 to 20% Ethyl acetate in hexane as an eluent) to afford tert-butyl 6-(6-bromonicotinoyl)-2,6-diazaspiro[3.3]heptane-2-carboxylate (520 mg, 68%), LCMS: 384.2[M+2]⁺

### Step-2: Synthesis of tert-butyl 6-(6-(4-chloroquinolin-6-yl)nicotinoyl)-2,6-diazaspiro[3.3]heptane-2-carboxylate

To a solution of tert-butyl 6-(6-bromonicotinoyl)-2,6-diazaspiro[3.3]heptane-2-carboxylate (150 mg, 0.39 mmol, 1.0 equiv) in 1,4-dioxane:water (3.5 mL:1.5 mL) was added K₂CO₃ (108 mg, 0.78 mmol, 2.0 equiv) and 4-chloro-6-(4,4,5,5-tetramethyl-1,3,2-dioxaborolan-2-yl)quinoline (113 mg, 0.392 mmol, 1 equiv). Reaction mixture was purged with nitrogen for 5 min, then was added PdCl₂dppf·DCM (16 mg, 0.019 mmol, 0.05 equiv). The reaction mixture was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure. The crude product was purified by flash chromatography (0 to 20% Ethyl acetate in hexane as an eluent) to afford tert-butyl 6-(6-(4-chloroquinolin-6-yl)nicotinoyl)-2,6-diazaspiro[3.3]heptane-2-carboxylate (110 mg, 60%), LCMS: 465.2[M+1]⁺.

### Step-3: Synthesis of tert-butyl 6-(6-(4-(benzo[d]thiazol-5-ylamino)quinolin-6-yl)nicotinoyl)-2,6-diazaspiro[3.3]heptane-2-carboxylate

To a solution of tert-butyl 6-(6-(4-chloroquinolin-6-yl)nicotinoyl)-2,6-diazaspiro[3.3]heptane-2-carboxylate (110 mg, 0.24 mmol, 1.0 equiv) in ethanol (5 mL) was added benzo[d]thiazol-5-amine (53 mg, 0.36 mmol, 1.5 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 5% MeOH in DCM as an eluent) to afford tert-butyl 6-(6-(4-(benzo[d]thiazol-5-ylamino)quinolin-6-yl)nicotinoyl)-2,6-diazaspiro[3.3]heptane-2-carboxylate (70 mg, 51%), LCMS: 579.1[M+1]⁺

### Step-4: Synthesis of (6-(4-(benzo[d]thiazol-5-ylamino)quinolin-6-yl)pyridin-3-yl)(2,6-diazaspiro[3.3]heptan-2-yl)methanone

To a solution of tert-butyl 6-(6-(4-(benzo[d]thiazol-5-ylamino)quinolin-6-yl)nicotinoyl)-2,6-diazaspiro[3.3]heptane-2-carboxylate (70 mg, 0.12 mmol, 1.0 equiv) in DCM (5 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (6-(4-(benzo[d]thiazol-5-ylamino)quinolin-6-yl)pyridin-3-yl)(2,6-diazaspiro[3.3]heptan-2-yl)methanone (9 mg, 16%), LCMS: 479.2[M+1]^{+ 1}H NMR (400 MHz, DMSO-d₆) δ 2.07 (br. s., 1H) 3.70 (br. s., 4H) 4.18 (br. s., 2H) 4.51 (br. s., 2H) 7.03 (s, 1H) 7.57 (s, 1H) 8.01 (s, 1H) 8.08 (br. s., 1H) 8.18-8.22 (d, 2H) 8.32 (s, 1H) 8.51 (br. s., 2H) 8.49 (s, 1H) 9.19 (s, 1H) 9.46 (d, J=9.54 Hz, 2H).

### Example 95: Synthesis of N-(6-(2-fluoro-4-(piperidin-4-yloxy)phenyl)quinolin-4-yl)benzo[d]thiazol-5-amine (Compound 295)

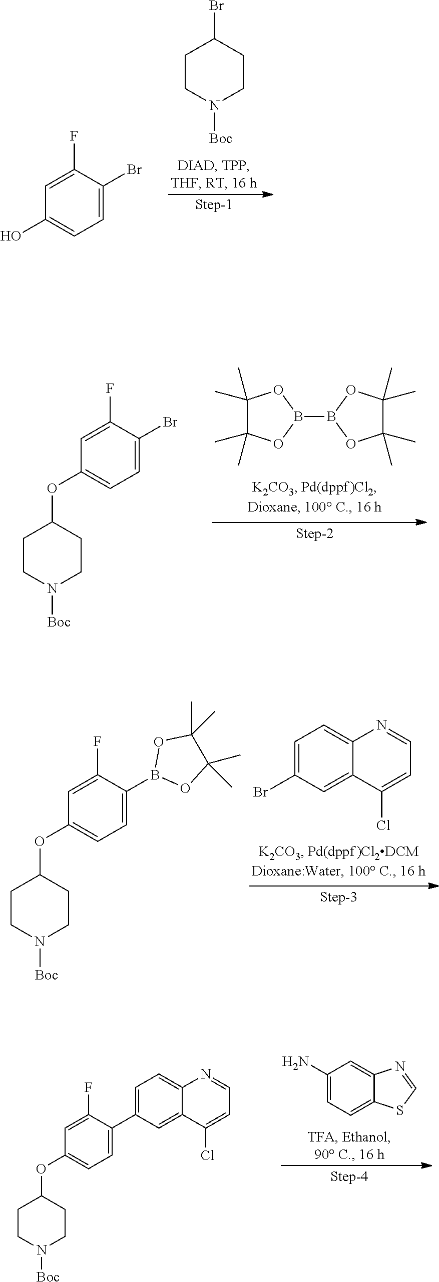

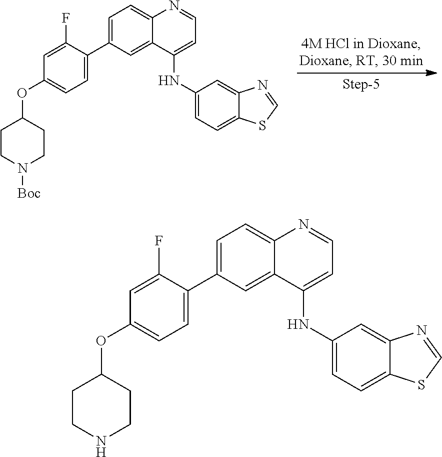 [SMILES: CC(C)(C)OC(=O)N1CCC(Oc2ccc(-c3ccc4nccc(Nc5ccc6scnc6c5)c4c3)c(F)c2)CC1 ; Fc1cc(OC2CCNCC2)ccc1-c1ccc2nccc(Nc3ccc4scnc4c3)c2c1]

### Step-1: Synthesis of tert-butyl 4-(4-bromo-3-fluorophenoxy)piperidine-1-carboxylate

To a stirred solution of TPP (2.05 g, 7.84 mmol, 1.5 equiv) in THF (7 mL), was added DIAD (500 mg, 2.29 mmol, 1.0 equiv) dropwise at 0° C. After precipitation, 4-bromo-3-fluorophenol (1 g, 5.23 mmol, 1.0 equiv) in THF (7 mL) and tert-butyl 4-bromopiperidine-1-carboxylate (1.2 g, 6.29 mmol, 1.2 equiv) were added. Resulting reaction mixture was stirred at RT for 16 h. The progress of the reaction was monitored by TLC and LCMS. Upon completion of reaction, reaction mixture was concentrated under reduced pressure. The resulting residue was suspended in hexane (200 mL), resulting white solid was filtered off. Filtrate was collected, diluted with ethyl acetate and washed with water (100 mL). Organic layers was dried over anhydrous Na₂SO₄, filtered and concentrated under reduced pressure. The crude product was purified by flash chromatography (0 to 2% EtOAc in hexane as an eluent) to afford tert-butyl 4-(4-bromo-3-fluorophenoxy)piperidine-1-carboxylate (1.2 g, 63%), LCMS: 374.1 [M+1]⁺.

### Step-2: Synthesis of tert-butyl 4-(3-fluoro-4-(4,4,5,5-tetramethyl-1,3,2-dioxaborolan-2-yl)phenoxy)piperidine-1-carboxylate

To a stirred solution of tert-butyl 4-(4-bromo-3-fluorophenoxy)piperidine-1-carboxylate (1.2 g, 3.21 mmol, 1.0 equiv) and Bis(pinacolato)diboron (977 mg, 3.85 mmol, 1.2 equiv) in 1,4-Dioxane (12 mL) was added KOAc (630 mg, 6.42 mmol, 2.0 equiv). The reaction was purged with N₂ for 10 min, then was added Pd(dppf)Cl₂ (117.5 mg, 0.16 mmol, 0.05 equiv). The resulting reaction mass was again purged with N₂ for 10 min. The reaction mixture was heated at 100° C. for 16 h. The progress of the reaction was monitored by LCMS. After completion of the reaction, reaction mixture was diluted with water (150 mL) and was extracted with EtOAc (2×250 mL). Combined organic layer was dried over Na₂SO₄, filtered and concentrated under reduced pressure to afford tert-butyl 4-(3-fluoro-4-(4,4,5,5-tetramethyl-1,3,2-dioxaborolan-2-yl)phenoxy)piperidine-1-carboxylate (1.0 g, 74%), LCMS: 366.2 [M+1]⁺ (Boronic-Acid Fragment).

### Step-3: Synthesis of tert-butyl 4-(4-(4-chloroquinolin-6-yl)-3-fluorophenoxy)piperidine-1-carboxylate

To a stirred solution of 6-bromo-4-chloroquinoline (200 mg, 0.82 mmol, 1.0 equiv) and tert-butyl 4-(3-fluoro-4-(4,4,5,5-tetramethyl-1,3,2-dioxaborolan-2-yl)phenoxy)piperidine-1-carboxylate (417 mg, 0.99 mmol, 1.2 equiv) in 1,4-dioxane:water (4 mL:0.5 mL) was added K₂CO₃ (228 mg, 1.65 mmol, 2.0 equiv). Reaction mixture was purged with N2 gas for 10 minutes and was added Pd(dppf)Cl2·DCM (34 mg, 0.041 mmol, 0.05 equiv). Reaction mixture was heated at 100° C. for 16 h. The progress of the reaction was monitored by LCMS and TLC. After completion of reaction, reaction mixture was diluted with water (75 mL) and was extracted with EtOAc (2×100 mL), combined organic layers were dried over Na₂SO₄, filtered and concentrated under reduced pressure. The crude product was purified by flash chromatography (eluted in 2% MeOH:DCM) to afford tert-butyl 4-(4-(4-chloroquinolin-6-yl)-3-fluorophenoxy)piperidine-1-carboxylate. (250 mg, 66%), LCMS: 457.2M+1]⁺.

### Step-4: Synthesis of tert-butyl 4-(4-(4-(benzo[d]thiazol-5-ylamino)quinolin-6-yl)-3-fluorophenoxy)piperidine-1-carboxylate

To a solution of tert-butyl 4-(4-(4-chloroquinolin-6-yl)-3-fluorophenoxy)piperidine-1-carboxylate (70 mg, 0.15 mmol, 1.0 equiv) and benzo[d]thiazol-5-amine (23.1 mg, 0.15 mmol, 1.0 equiv) in EtOH (3 mL) was added TFA (0.05 mL) and the reaction mixture was stirred at 90° C. for 16 h. The progress of reaction was monitored by TLC and LCMS. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to afford tert-butyl 4-(4-(4-(benzol[d]thiazol-5-ylamino)quinolin-6-yl)-3-fluorophenoxy)piperidine-1-carboxylate (50 mg, 57%), LCMS: 571.2 [M+1]⁺.

### Step-5: Synthesis of N-(6-(2-fluoro-4-(piperidin-4-yloxy)phenyl)quinolin-4-yl)benzo[d]thiazol-5-amine

To a solution of tert-butyl 4-(4-(4-(benzo[d]thiazol-5-ylamino)quinolin-6-yl)-3-fluorophenoxy)piperidine-1-carboxylate (70 mg, 0.123 mmol, 1.0 equiv) in dioxane (3 mL), was added 4M HCl in Dioxane (1 mL) at 0° C. The reaction mixture was stirred at RT for 30 minutes. The progress of reaction was monitored by TLC and LCMS. After completion of reaction, reaction mixture was concentrated under reduced pressure. The crude product was washed with diethyl ether (2×10 mL) and dried under reduced pressure to afford N-(6-(2-fluoro-4-(piperidin-4-yloxy)phenyl)quinolin-4-yl)benzo[d]thiazol-5-amine as a hydrochloride salt (14 mg, 24%), LCMS: 471.2 [M+1]⁺, ¹H NMR (400 MHz, DMSO-d₆) δ 9.54 (s, 1H) 8.96 (s, 1H) 8.81 (br. s., 2H) 8.54 (d, J=6.99 Hz, 1H) 8.40 (d, J=8.27 Hz, 1H) 8.18-8.27 (m, 2H) 8.12 (d, J=8.90 Hz, 1H) 7.74 (t, J=8.90 Hz, 1H) 7.63 (d, J=10.17 Hz, 1H) 7.19 (d, J=13.35 Hz, 1H) 7.07-7.12 (m, 1H) 6.93 (d, J=6.99 Hz, 1H) 4.80 (br. s., 1H) 3.26 (br. s., 2H) 3.10 (br. s., 2H) 2.13 (br. s., 2H) 1.88 (d, J=9.54 Hz, 2H).

### Example 96: Synthesis of (4-(4-(benzo[d]thiazol-5-ylamino)quinolin-6-yl)-5-fluoro-2-methylphenyl)(piperazin-1-yl)methanone (Compound 196)

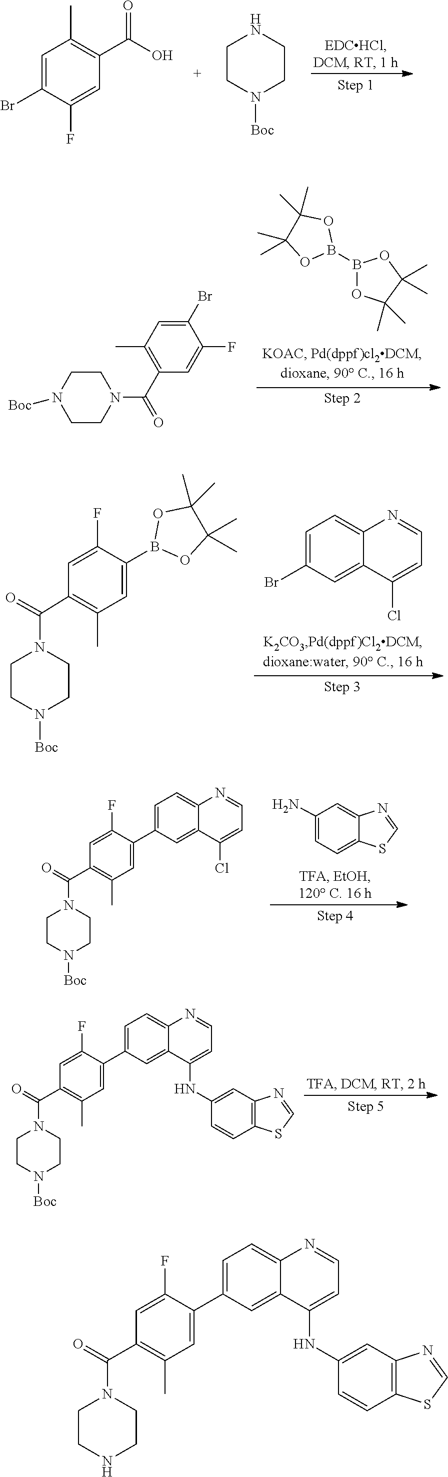

### Step-1: Synthesis of tert-butyl 4-(4-bromo-5-fluoro-2-methylbenzoyl)piperazine-1-carboxylate

To a solution 4-bromo-5-fluoro-2-methylbenzoic acid (400 mg, 1.71 mmol, 1.0 equiv) in DCM (15 mL) was added EDC·HCl (428 mg, 2.22 mmol, 1.3 equiv) and tert-butyl piperazine-1-carboxylate (383 mg, 2.06 mmol, 1.2 equiv). Reaction mixture was stirred at RT for 1 h. The progress of reaction was monitored by TLC. After completion of reaction, reaction mixture was diluted with water (30 mL) and extracted with DCM (2×60 ml), washed with brine solution, dried over anhydrous sodium sulphate, filtered and concentrated under reduced pressure to afford tert-butyl 4-(4-bromo-5-fluoro-2-methylbenzoyl)piperazine-1-carboxylate (650 mg, 94%), LCMS: 401.1[M+1]⁺.

### Step-2: Synthesis of tert-butyl 4-(5-fluoro-2-methyl-4-(4,4,5,5-tetramethyl-1,3,2-dioxaborolan-2-yl)benzoyl)piperazine-1-carboxylate

To a solution tert-butyl 4-(4-bromo-5-fluoro-2-methylbenzoyl)piperazine-1-carboxylate (650 mg, 1.62 mmol, 1.0 equiv) in 1,4-dioxane (10 mL) was added KOAc (476 mg, 4.86 mmol, 3.0 equiv) and 4,4,4′,4′,5,5,5′,5′-octamethyl-2,2′-bi(1,3,2-dioxaborolane) (614 mg, 2.43 mmol, 1.5 equiv). Reaction mixture was purged with nitrogen for 5 min, was added PdCl₂dppf·DCM (66 mg, 0.08 mmol, 0.05 equiv). The reaction was heated at 90° C. for 16 h. The progress of reaction was monitored by TLC. After completion of reaction, reaction mixture was diluted with water (60 mL) and extracted with ethyl acetate (2×120 mL), dried over anhydrous sodium sulphate, filtered and concentrated under reduced pressure. The crude product was purified by flash chromatography (0 to 2% EtOAc in hexane as an eluent) to afford tert-butyl 4-(5-fluoro-2-methyl-4-(4,4,5,5-tetramethyl-1,3,2-dioxaborolan-2-yl)benzoyl)piperazine-1-carboxylate (700 mg, 96%), LCMS: 311.2[M+1]⁺.[Boronic acid fragment].

### Step-3: Synthesis of tert-butyl 4-(4-(4-chloroquinolin-6-yl)-5-fluoro-2-methylbenzoyl)piperazine-1-carboxylate

To a solution of tert-butyl 4-(5-fluoro-2-methyl-4-(4,4,5,5-tetramethyl-1,3,2-dioxaborolan-2-yl)benzoyl)piperazine-1-carboxylate (350 mg, 0.781 mmol, 1 equiv) in 1,4-dioxane:water (8.5 ml:1.5 ml) was added K₂CO₃ (161 mg, 1.171 mmol, 1.5 equiv) and 6-bromo-4-chloroquinoline (227 mg, 0.94 mmol, 1.2 equiv). Reaction mixture was purged with nitrogen for 5 min, then was added PdCl₂dppf·DCM (31 mg, 0.039 mmol, 0.05 equiv). The reaction was heated at 90° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure to afford tert-butyl 4-(4-(4-chloroquinolin-6-yl)-5-fluoro-2-methylbenzoyl)piperazine-1-carboxylate (150 mg, 40%), LCMS: 484.1 [M+1]⁺.

### Step-4: Synthesis of tert-butyl 4-(4-(4-(benzo[d]thiazol-5-ylamino)quinolin-6-yl)-5-fluoro-2-methylbenzoyl)piperazine-1-carboxylate

To a solution of tert-butyl 4-(4-(4-chloroquinolin-6-yl)-5-fluoro-2-methylbenzoyl)piperazine-1-carboxylate (150 mg, 0.31 mmol, 1.0 equiv) in ethanol (5 mL) was added benzo[d]thiazol-5-amine (47 mg, 0.310 mmol, 1.0 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to afford tert-butyl 4-(4-(4-(benzo[d]thiazol-5-ylamino)quinolin-6-yl)-5-fluoro-2-methylbenzoyl)piperazine-1-carboxylate (100 mg, 54%), LCMS: 598.1[M+1]⁺.

### Step-5: Synthesis of (4-(4-(benzo[d]thiazol-5-ylamino)quinolin-6-yl)-5-fluoro-2-methylphenyl)(piperazin-1-yl)methanone

To a solution tert-butyl 4-(4-(4-(benzo[d]thiazol-5-ylamino)quinolin-6-yl)-5-fluoro-2-methylbenzoyl)piperazine-1-carboxylate (50 mg, 0.08 mmol, 1.0 equiv) in dichloromethane (10 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-6-yl)-5-fluoro-2-methylphenyl)(piperazin-1-yl)methanone (10 mg, 24%), LCMS: 498.1[M+1]⁺. ¹H NMR (400 MHz, DMSO-d₆) δ 2.26-2.34 (m, 2H) 2.67 (s, 1H) 2.86 (br. s., 1H) 3.20 (br. s., 3H) 3.65 (br. s., 2H) 6.30 (br. s., 1H) 6.55 (br. s., 2H) 7.05 (d, J=5.09 Hz, 1H) 7.29 (d, J=10.81 Hz, 1H) 7.54 (s, 1H) 7.62 (d, J=7.63 Hz, 1H) 7.88-7.92 (m, 1H) 7.97 (s, 1H) 8.04 (s, 1H) 8.15 (s, 1H) 8.21 (d, J=8.27 Hz, 1H) 8.51 (d, J=5.72 Hz, 1H) 8.62 (s, 1H) 9.43 (s, 1H).

### Example 97: Synthesis of (4-(4-(benzo[d]thiazol-5-ylamino)quinolin-7-yl)-5-fluoro-2-methylphenyl)(piperazin-1-yl)methanone (Compound 297)

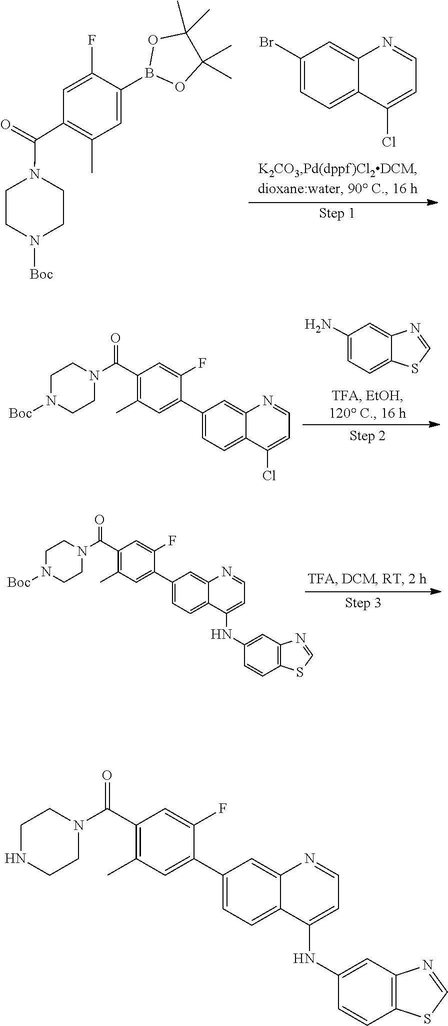 [SMILES: Cc1cc(B2OC(C)(C)C(C)(C)O2)c(F)cc1C(=O)N1CCN(C(=O)OC(C)(C)C)CC1 ; Clc1ccnc2cc(Br)ccc12 ; Cc1cc(-c2ccc3c(Cl)ccnc3c2)c(F)cc1C(=O)N1CCN(C(=O)OC(C)(C)C)CC1 ; Nc1ccc2scnc2c1 ; Cc1cc(-c2ccc3c(Nc4ccc5scnc5c4)ccnc3c2)c(F)cc1C(=O)N1CCN(C(=O)OC(C)(C)C)CC1 ; Cc1cc(-c2ccc3c(Nc4ccc5scnc5c4)ccnc3c2)c(F)cc1C(=O)N1CCNCC1]

### Step-1: Synthesis of tert-butyl 4-(4-(4-chloroquinolin-7-yl)-5-fluoro-2-methylbenzoyl)piperazine-1-carboxylate

To a solution of tert-butyl 4-(5-fluoro-2-methyl-4-(4,4,5,5-tetramethyl-1,3,2-dioxaborolan-2-yl)benzoyl)piperazine-1-carboxylate (650 mg, 1.45 mmol, 1.0 equiv) in 1,4-dioxane:water (8.5 ml:1.5 ml) was added K₂CO₃ (300 mg, 2.17 mmol, 1.5 equiv) and 7-bromo-4-chloroquinoline (422 mg, 1.74 mmol, 1.2 equiv). Reaction mixture was purged with nitrogen for 5 min, was added PdCl₂dppf·DCM (59 mg, 0.07 mmol, 0.05 equiv). The reaction was heated at 90° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure to afford tert-butyl 4-(4-(4-chloroquinolin-7-yl)-5-fluoro-2-methylbenzoyl)piperazine-1-carboxylate (350 mg, 50%), LCMS: 484.2 [M+1]⁺.

### Step-2: Synthesis of tert-butyl 4-(4-(4-(benzo[d]thiazol-5-ylamino)quinolin-7-yl)-5-fluoro-2-methylbenzoyl)piperazine-1-carboxylate

To a solution of tert-butyl 4-(4-(4-chloroquinolin-7-yl)-5-fluoro-2-methylbenzoyl)piperazine-1-carboxylate (350 mg, 0.72 mmol, 1.0 equiv) in ethanol (10 mL) was added benzo[d]thiazol-5-amine (108 mg, 0.72 mmol, 1.0 equiv) and TFA (0.15 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to afford tert-butyl 4-(4-(4-(benzo[d]thiazol-5-ylamino)quinolin-7-yl)-5-fluoro-2-methylbenzoyl)piperazine-1-carboxylate (100 mg, 23%), LCMS: 598.1[M+1]⁺.

### Step-3: Synthesis of (4-(4-(benzo[d]thiazol-5-ylamino)quinolin-7-yl)-5-fluoro-2-methylphenyl)(piperazin-1-yl)methanone

To a solution tert-butyl 4-(4-(4-(benzo[d]thiazol-5-ylamino)quinolin-7-yl)-5-fluoro-2-methylbenzoyl)piperazine-1-carboxylate (100 mg, 0.17 mmol, 1.0 equiv) in dichloromethane (5 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo [d]thiazol-5-ylamino)quinolin-7-yl)-5-fluoro-2-methylphenyl)(piperazin-1-yl)methanone (50 mg, 60%), LCMS: 498.2[M+1]⁺. ¹H NMR (400 MHz, DMSO-d₆) δ 1.23 (s, 1H) 2.29-2.33 (m, 3H) 2.67 (br. s., 2H) 2.85 (br. s., 2H) 3.21 (br. s., 2H) 3.65 (br. s., 2H) 7.04 (d, J=5.09 Hz, 1H) 7.28 (d, J=10.81 Hz, 1H) 7.56 (d, J=8.90 Hz, 1H) 7.65 (d, J=7.63 Hz, 1H) 7.77 (d, J=8.90 Hz, 1H) 8.08 (s, 2H) 8.20 (d, J=8.27 Hz, 1H) 8.53 (d, J=7.63 Hz, 2H) 9.28 (br. s., 1H) 9.43 (s, 1H).

### Example 98: Synthesis of (4-(4-(benzo[d]thiazol-5-ylamino)quinolin-6-yl)-3-fluorophenyl)(hexahydropyrrolo[3,4-c]pyrrol-2(1H)-yl)methanone (Compound 298)

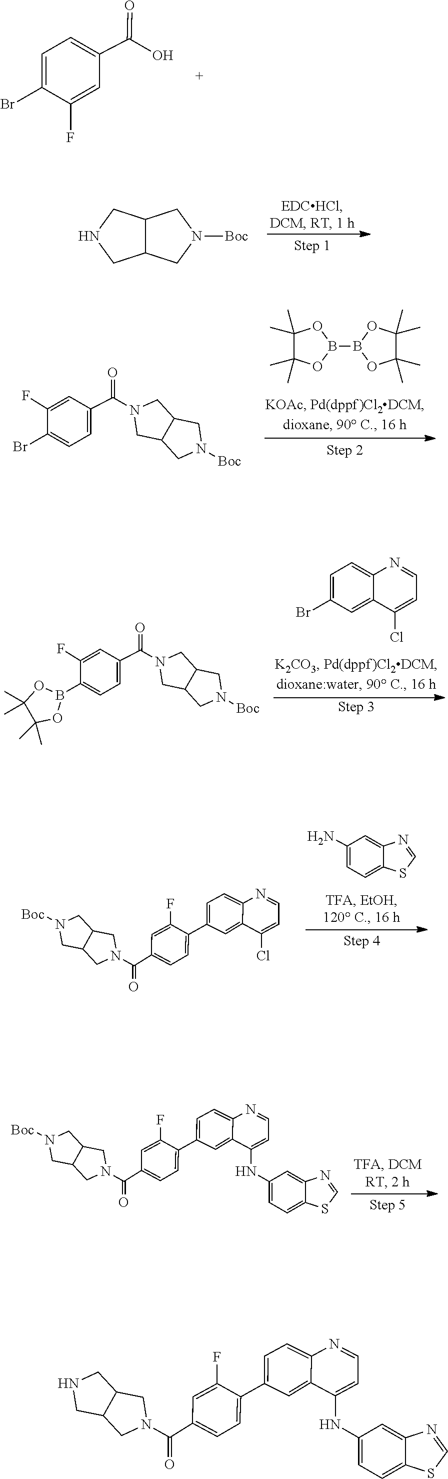

### Step-1: Synthesis of tert-butyl 5-(4-bromo-3-fluorobenzoyl)hexahydropyrrolo[3,4-c]pyrrole-2(1H)-carboxylate

To a solution 4-bromo-3-fluorobenzoic acid (300 mg, 1.37 mmol, 1.0 equiv) in DCM (15 mL) was added EDC·HCl (341 mg, 1.78 mmol, 1.3 equiv) and tert-butyl hexahydropyrrolo[3,4-c]pyrrole-2(1H)-carboxylate (290 mg, 1.37 mmol, 1.0 equiv). Reaction mixture was stirred at RT for 1 h. The progress of reaction was monitored by TLC. After completion of reaction, reaction mixture was diluted with water (30 mL) and extracted with DCM (2×60 ml), washed with brine solution, dried over anhydrous sodium sulphate, filtered and concentrated under reduced pressure to afford tert-butyl 5-(4-bromo-3-fluorobenzoyl)hexahydropyrrolo[3,4-c]pyrrole-2(1H)-carboxylate (550 gm, 97%), LCMS: 413.2 [M+1]⁺

### Step-2: Synthesis of tert-butyl 5-(3-fluoro-4-(4,4,5,5-tetramethyl-1,3,2-dioxaborolan-2-yl)benzoyl)hexahydropyrrolo[3,4-c]pyrrole-2(1H)-carboxylate

To a solution tert-butyl 5-(4-bromo-3-fluorobenzoyl)hexahydropyrrolo[3,4-c]pyrrole-2(1H)-carboxylate (550 mg, 1.33 mmol, 1.0 equiv) in 1,4-dioxane (10 mL) was added KOAc (392 mg, 4.00 mmol, 3.0 equiv) and 4,4,4′,4′,5,5,5′,5′-octamethyl-2,2′-bi(1,3,2-dioxaborolane) (372 mg, 1.47 mmol, 1.1 equiv). Reaction mixture was purged with nitrogen for 5 min, then was added PdCl₂dppf·DCM (54 mg, 0.07 mmol, 0.05 equiv). The reaction was heated at 90° C. for 16 h. The progress of reaction was monitored by TLC. After completion of reaction, reaction mixture was diluted with water (60 mL) and extracted with ethyl acetate (2×120 mL), dried over anhydrous sodium sulphate, filtered and concentrated under reduced pressure. The crude product was purified by flash chromatography (0 to 20% EtOAc in hexane as an eluent) to afford tert-butyl 5-(3-fluoro-4-(4,4,5,5-tetramethyl-1,3,2-dioxaborolan-2-yl)benzoyl)hexahydropyrrolo[3,4-c]pyrrole-2(1H)-carboxylate (600 gm, 98%), LCMS: 323.2 [M+1]⁺.[Boronic acid fragment].

### Step-3: Synthesis of tert-butyl 5-(4-(4-chloroquinolin-6-yl)-3-fluorobenzoyl)hexahydropyrrolo[3,4-c]pyrrole-2(1H)-carboxylate

To a solution of tert-butyl 5-(3-fluoro-4-(4,4,5,5-tetramethyl-1,3,2-dioxaborolan-2-yl)benzoyl)hexahydropyrrolo[3,4-c]pyrrole-2(1H)-carboxylate (600 mg, 1.30 mmol, 1.0 equiv) in 1,4-dioxane:water (8.5 ml:1.5 ml) was added K₂CO₃ (270 mg, 1.95 mmol, 1.5 equiv) and 6-bromo-4-chloroquinoline (379 mg, 1.56 mmol, 1.2 equiv). Reaction mixture was purged with nitrogen for 5 min, was added PdCl₂dppf·DCM (53 mg, 0.06 mmol, 0.05 equiv). The reaction mixture was heated at 90° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 20% EtOAc in hexane as an eluent) to afford tert-butyl 5-(4-(4-chloroquinolin-6-yl)-3-fluorobenzoyl)hexahydropyrrolo[3,4-c]pyrrole-2(1H)-carboxylate (350 mg, 54%), LCMS: 496.1 [M+1]⁺.

### Step-4: Synthesis of tert-butyl 5-(4-(4-(benzo[d]thiazol-5-ylamino)quinolin-6-yl)-3-fluorobenzoyl)hexahydropyrrolo[3,4-c]pyrrole-2(1H)-carboxylate

To a solution of tert-butyl 5-(4-(4-chloroquinolin-6-yl)-3-fluorobenzoyl)hexahydropyrrolo[3,4-c]pyrrole-2(1H)-carboxylate (350 mg, 0.70 mmol, 1.0 equiv) in ethanol (10 mL) was added benzo[d]thiazol-5-amine (106 mg, 0.70 mmol, 1.0 equiv) and TFA (0.2 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to afford tert-butyl 5-(4-(4-(benzo[d]thiazol-5-ylamino)quinolin-6-yl)-3-fluorobenzoyl)hexahydropyrrolo[3,4-c]pyrrole-2(1H)-carboxylate (200 mg, 46%), LCMS: 610.3[M+1]⁺.

### Step-5: Synthesis of (4-(4-(benzo[d]thiazol-5-ylamino)quinolin-6-yl)-3-fluorophenyl)(hexahydropyrrolo[3,4-c]pyrrol-2(1H)-yl)methanone

To a solution tert-butyl 5-(4-(4-(benzo[d]thiazol-5-ylamino)quinolin-6-yl)-3-fluorobenzoyl)hexahydropyrrolo[3,4-c]pyrrole-2(1H)-carboxylate (100 mg, 0.16 mmol, 1.0 equiv) in dichloromethane (5 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-6-yl)-3-fluorophenyl)(hexahydropyrrolo[3,4-c]pyrrol-2(1H)-yl)methanone (50 mg, 60%), LCMS: 510.1[M+1]⁺. ¹H NMR (400 MHz, DMSO-d₆) δ 1.09 (t, J=6.99 Hz, 1H) 1.23 (br. s., 1H) 3.04 (br. s., 2H) 3.14 (br. s., 1H) 3.39 (br. s., 3H) 3.60 (br. s., 1H) 3.73 (br. s., 2H) 7.06 (d, J=3.81 Hz, 1H) 7.48-7.57 (m, 3H) 7.83 (t, J=7.95 Hz, 1H) 7.89-7.94 (m, 1H) 7.97-8.05 (m, 2H) 8.21 (d, J=8.90 Hz, 1H) 8.52 (d, J=4.45 Hz, 1H) 8.66 (br. s., 1H) 9.32 (br. s., 1H) 9.44 (s, 1H).

### Example 99: Synthesis of N-(6-(2-fluoro-4-((hexahydropyrrolo[3,4-c]pyrrol-2(1H)-yl)methyl)phenyl)quinolin-4-yl)benzo[d]thiazol-5-amine (Compound 299)

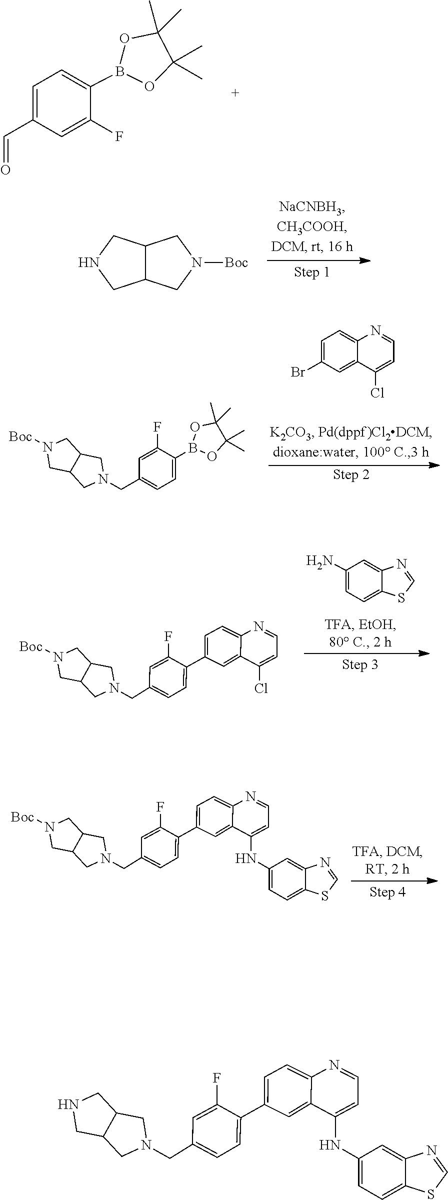

### Step-1: Synthesis of tert-butyl 5-(3-fluoro-4-(4,4,5,5-tetramethyl-1,3,2-dioxaborolan-2-yl)benzyl)hexahydropyrrolo[3,4-c]pyrrole-2(1H)-carboxylate

To a solution of 3-fluoro-4-(4,4,5,5-tetramethyl-1,3,2-dioxaborolan-2-yl)benzaldehyde (3.0 g, 0.80 mmol, 1.0 equiv) in MeOH (30 mL) was added CH₃COOH (1 mL) and tert-butyl hexahydropyrrolo[3,4-c]pyrrole-2(1H)-carboxylate (3.82 g, 18 mmol, 1.5 equiv). Reaction mixture was stirred at RT for 16 h. NaCNBH3 (1.13 g, 1.5 mmol, 1.5 equiv) was added at 0° C. Reaction was stirred at RT for overnight. The progress of reaction was monitored by TLC. After completion of reaction, reaction mixture was diluted with water (30 mL) and extracted with DCM (2×60 ml), washed with brine solution, dried over anhydrous sodium sulphate, filtered and concentrated under reduced pressure. The crude product was purified by flash chromatography (0-20% Ethyl acetate in hexane as an eluent) to afford tert-butyl 5-(3-fluoro-4-(4,4,5,5-tetramethyl-1,3,2-dioxaborolan-2-yl)benzyl)hexahydropyrrolo[3,4-c]pyrrole-2(1H)-carboxylate (4.0 g, 74%), LCMS: 447.3[M+1]⁺[Boronic acid fragment].

### Step-2: Synthesis of tert-butyl 5-(4-(4-chloroquinolin-6-yl)-3-fluorobenzyl)hexahydropyrrolo[3,4-c]pyrrole-2(1H)-carboxylate

To a solution tert-butyl 5-(3-fluoro-4-(4,4,5,5-tetramethyl-1,3,2-dioxaborolan-2-yl)benzyl)hexahydropyrrolo[3,4-c]pyrrole-2(1H)-carboxylate (2.0 g, 4.48 mmol, 1.0 equiv) in 1,4-dioxane:water (18 mL:2.5 mL) was added K₂CO₃ (1.54 g, 11.2 mmol, 2.5 equiv) and 6-bromo-4-chloroquinoline (1.30 g, 5.37 mmol, 1.2 equiv). Reaction mixture was purged with nitrogen for 5 min, then was added PdCl₂dppf·DCM (183 mg, 0.22 mmol, 0.05 equiv). The reaction mixture was heated at 100° C. for 3 h. The progress of reaction was monitored by TLC. After completion of reaction, reaction mixture was diluted with water (40 mL) and extracted with ethyl acetate (2×100 mL), dried over anhydrous sodium sulphate, filtered and concentrated under reduced pressure. The crude product was purified by flash chromatography (0 to 20% EtOAc in hexane as an eluent) to afford tert-butyl 5-(4-(4-chloroquinolin-6-yl)-3-fluorobenzyl)hexahydropyrrolo[3,4-c]pyrrole-2(1H)-carboxylate (800 mg, 37%), LCMS: 482.2[M+1]⁺.

### Step-3: Synthesis of tert-butyl 5-(4-(4-(benzo[d]thiazol-5-ylamino)quinolin-6-yl)-3-fluorobenzyl)hexahydropyrrolo[3,4-c]pyrrole-2(1H)-carboxylate

To a solution of afford tert-butyl 5-(4-(4-chloroquinolin-6-yl)-3-fluorobenzyl)hexahydropyrrolo[3,4-c]pyrrole-2(1H)-carboxylate (800 mg, 1.66 mmol, 1.0 equiv) in ethanol (10 mL) was added benzo[d]thiazol-5-amine (249 mg, 1.66 mmol, 1.0 equiv) and TFA (0.4 mL). The reaction mixture was heated at 80° C. for 2 h. After completion of reaction, reaction mixture was concentrated under reduced pressure. The resulting residue was basified with saturated sodium bicarbonate solution (20 mL), was extracted with ethyl acetate (2×60 mL), dried over anhydrous sodium sulphate, filtered and concentrated under reduced pressure. The crude product was purified by flash column chromatography (0 to 6% MeOH in DCM as an eluent) to afford tert-butyl 5-(4-(4-(benzo[d]thiazol-5-ylamino)quinolin-6-yl)-3-fluorobenzyl)hexahydropyrrolo[3,4-c]pyrrole-2(1H)-carboxylate (400 mg, 46%), LCMS: 496.1[M+1]⁺.

### Step-4: Synthesis N-(6-(2-fluoro-4-((hexahydropyrrolo[3,4-c]pyrrol-2(1H)-yl)methyl)phenyl)quinolin-4-yl)benzo[d]thiazol-5-amine

To a solution of tert-butyl 5-(4-(4-(benzo[d]thiazol-5-ylamino)quinolin-6-yl)-3-fluorobenzyl)hexahydropyrrolo[3,4-c]pyrrole-2(1H)-carboxylate (200 mg, 0.335 mmol, 1.0 equiv) in dichloromethane (10 mL) was added TFA (0.5 mL) at 0° C. The reaction mixture was stirred at RT for 2 h. After completion of reaction, reaction mixture was concentrated under reduced pressure. The residue was basified with saturated sodium bicarbonate solution (50 mL), extracted with 5% MEOH/DCM (2×30 mL), dried over anhydrous sodium sulphate, filtered and concentrated under reduced pressure. The resulting solid was washed with diethyl ether and dried under vacuum to afford N-(6-(2-fluoro-4-((hexahydropyrrolo[3,4-c]pyrrol-2(1H)-yl)methyl)phenyl)quinolin-4-yl)benzo[d]thiazol-5-amine (50 mg, 30%), LCMS: 496.2 [M+1]^{+ 1}H NMR (400 MHz, DMSO-d₆) δ 9.43 (s, 1H), 9.27 (s, 1H), 8.60 (s, 1H), 8.51 (d, J=5.09 Hz, 1H), 8.20 (d, J=8.27 Hz, 1H), 8.03 (s, 1H), 7.93-8.01 (m, 1H), 7.89 (d, J=7.63 Hz, 1H), 7.67 (t, J=7.95 Hz, 1H), 7.55 (d, J=7.63 Hz, 1H), 7.24-7.36 (m, 2H), 7.05 (d, J=5.09 Hz, 1H), 3.60 (s, 2H), 2.87 (br. s., 2H), 2.59 (d, J=8.27 Hz, 4H), 2.35 (d, J=8.90 Hz, 2H).

### Example 100: Synthesis of N-(7-(2-fluoro-4-((hexahydropyrrolo[3,4-c]pyrrol-2(1H)-yl)methyl)phenyl)quinolin-4-yl)benzo[d]thiazol-5-amine (Compound 300)

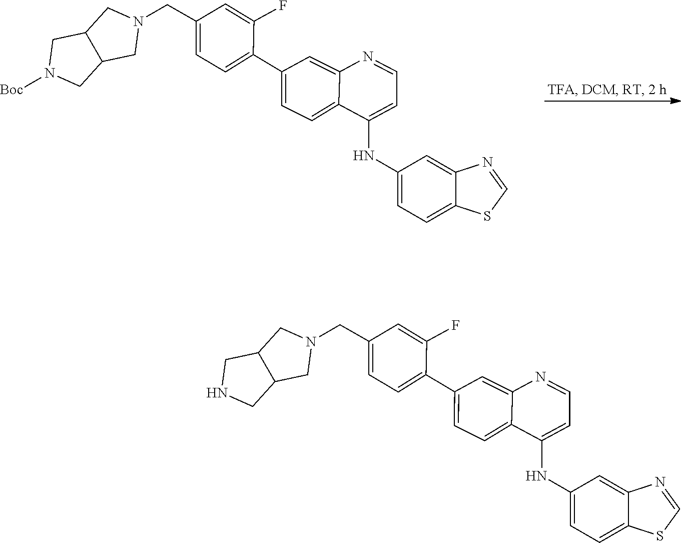 [SMILES: CC(C)(C)OC(=O)N1CC2CN(Cc3ccc(-c4ccc5c(Nc6ccc7scnc7c6)ccnc5c4)c(F)c3)CC2C1 ; Fc1cc(CN2CC3CNCC3C2)ccc1-c1ccc2c(Nc3ccc4scnc4c3)ccnc2c1]

### Step-4: Synthesis N-(7-(2-fluoro-4-((hexahydropyrrolo[3,4-c]pyrrol-2(1H)-yl)methyl)phenyl)quinolin-4-yl)benzo[d]thiazol-5-amine

To a solution of tert-butyl 5-(4-(4-(benzo[d]thiazol-5-ylamino)quinolin-6-yl)-3-fluorobenzyl)hexahydropyrrolo[3,4-c]pyrrole-2(1H)-carboxylate (50 mg, 0.08 mmol, 1.0 equiv) in dichloromethane (5 mL) was added TFA (0.5 mL) at 0° C. The reaction mixture was stirred at RT for 2 h. After completion of reaction, reaction mixture was concentrated under reduced pressure. The residue was basified with saturated sodium bicarbonate solution (50 mL), extracted with 5% MEOH/DCM (2×30 mL), dried over anhydrous sodium sulphate, filtered and concentrated under reduced pressure. The resulting solid was washed with diethyl ether and dried under vacuum to afford N-(7-(2-fluoro-4-((hexahydropyrrolo[3,4-c]pyrrol-2(1H)-yl)methyl)phenyl)quinolin-4-yl)benzo[d]thiazol-5-amine (10 mg, 24%), LCMS: 496.2 [M+1]^{+ 1}H NMR (400 MHz, DMSO-d₆) δ 9.43 (s, 1H), 9.27 (s, 1H), 8.60 (s, 1H), 8.51 (d, J=5.09 Hz, 1H), 8.20 (d, J=8.27 Hz, 1H), 8.03 (s, 1H), 7.93-8.01 (m, 1H), 7.89 (d, J=7.63 Hz, 1H), 7.67 (t, J=7.95 Hz, 1H), 7.55 (d, J=7.63 Hz, 1H), 7.24-7.36 (m, 2H), 7.05 (d, J=5.09 Hz, 1H), 3.60 (s, 2H), 2.87 (br. s., 2H), 2.59 (d, J=8.27 Hz, 4H), 2.35 (d, J=8.88 Hz, 2H).

### Example 101: Synthesis of (4-(4-(benzo[d]thiazol-5-ylamino)quinolin-7-yl)-3-fluorophenyl)((1S,4S)-2,5-diazabicyclo[2.2.1]heptan-2-yl)methanone (Compound 301)

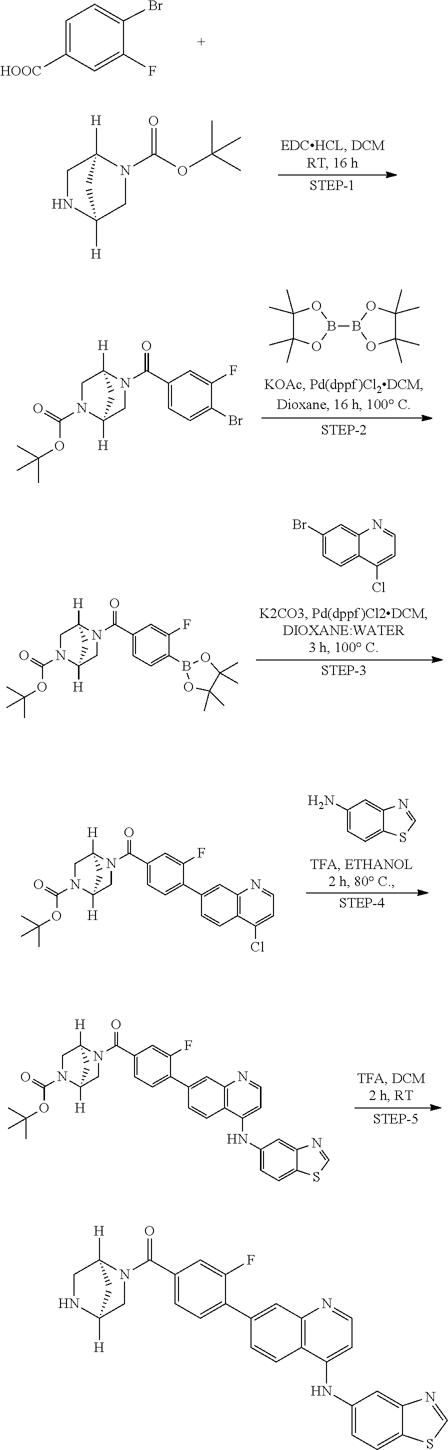

### Step-1: Synthesis of (1S,4S)-tert-butyl 5-(4-bromo-3-fluorobenzoyl)-2,5-diazabicyclo[2.2.1]heptane-2-carboxylate

To a stirred solution of 4-bromo-3-fluorobenzoic acid (500 mg, 2.28 mmol, 1.0 equiv) in DCM (10 mL), was added EDC·HCl (568 mg, 2.96 mmol, 1.3 equiv). Reaction was stirred at RT for 1 h. Then (1S,4S)-tert-butyl 2,5-diazabicyclo[2.2.1]heptane-2-carboxylate (543 mg, 2.74 mmol, 1.2 equiv) was added. Resulting reaction mixture was stirred at RT for 16 h. The progress of the reaction was monitored by TLC and LCMS. After completion of reaction, reaction mixture was diluted with water (30 mL) and extracted with DCM (2×60 ml), washed with brine solution, dried over anhydrous sodium sulphate, filtered and concentrated under reduced pressure to afford 1S,4S)-tert-butyl 5-(4-bromo-3-fluorobenzoyl)-2,5-diazabicyclo[2.2.1]heptane-2-carboxylate (680 mg, 74%), LCMS: 399.1 [M+1]⁺

### Step-2: Synthesis of (1S,4S)-tert-butyl 5-(3-fluoro-4-(4,4,5,5-tetramethyl-1,3,2-dioxaborolan-2-yl)benzoyl)-2,5-diazabicyclo[2.2.1]heptane-2-carboxylate

To a stirred solution of 1S,4S)-tert-butyl 5-(4-bromo-3-fluorobenzoyl)-2,5-diazabicyclo[2.2.1]heptane-2-carboxylate (680 mg, 1.70 mmol, 1.0 equiv) and Bis(pinacolato)diboron (519 mg, 2.04 mmol, 1.2 equiv) in 1,4-Dioxane (10 mL), we added KOAc (333 mg, 3.40 mmol, 2.0 equiv). The reaction was purged with N₂ for 10 min. Then was added Pd(dppf)Cl₂·DCM (69 mg, 0.08 mmol, 0.05 equiv). The resulting reaction mixture was again purged with N₂ for 10 minutes and stirred at 100° C. for 16 h. The progress of the reaction was monitored by LCMS and TLC. After completion of reaction, reaction mixture was diluted with water (60 mL) and extracted with ethyl acetate (2×120 mL), dried over anhydrous sodium sulphate, filtered and concentrated under reduced pressure. The crude product was purified by flash chromatography (0 to 20% EtOAc in hexane as an eluent) to afford (1S,4S)-tert-butyl 5-(3-fluoro-4-(4,4,5,5-tetramethyl-1,3,2-dioxaborolan-2-yl)benzoyl)-2,5-diazabicyclo[2.2.1]heptane-2-carboxylate (750 mg, 98%), LCMS: 309.2 [M+1]⁺ (Boronic acid Fragment) Step-3: Synthesis of (1S,4S)-tert-butyl 5-(4-(4-chloroquinolin-7-yl)-3-fluorobenzoyl)-2,5-diazabicyclo[2.2.1]heptane-2-carboxylate

To a stirred solution of (1S,4S)-tert-butyl 5-(3-fluoro-4-(4,4,5,5-tetramethyl-1,3,2-dioxaborolan-2-yl)benzoyl)-2,5-diazabicyclo[2.2.1]heptane-2-carboxylate (750 mg, 1.68 mmol, 1.0 equiv) and 7-bromo-4-chloroquinoline (489 mg, 2.01 mmol, 1.2 equiv) in 1,4-dioxane:water (8.5 mL:1.5 mL), was added K₂CO₃ (580 mg, 4.20 mmol, 2.5 equiv). Reaction mixture was purged with N2 gas for 10 min, then was added Pd(dppf)Cl2·DCM (69 mg, 0.08 mmol, 0.05 equiv). Reaction mixture was stirred at 100° C. for 3 h. The progress of the reaction was monitored by LCMS and TLC. After completion of reaction, reaction mixture was diluted with water (40 mL) and extracted with ethyl acetate (2×75 mL), dried over anhydrous sodium sulphate, filtered and concentrated under reduced pressure. The crude product was purified by flash chromatography (0-2% MeOH:DCM as an eluent) to afford (1S,4S)-tert-butyl 5-(4-(4-chloroquinolin-7-yl)-3-fluorobenzoyl)-2,5-diazabicyclo[2.2.1]heptane-2-carboxylate (300 mg, 37%). LCMS: 482.3 [M+1]⁺.

### Step-4: Synthesis of (1S,4S)-tert-butyl 5-(4-(4-(benzo[d]thiazol-5-ylamino)quinolin-7-yl)-3-fluorobenzoyl)-2,5-diazabicyclo[2.2.1]heptane-2-carboxylate

To a solution of (1S,4S)-tert-butyl 5-(4-(4-chloroquinolin-7-yl)-3-fluorobenzoyl)-2,5-diazabicyclo[2.2.1]heptane-2-carboxylate (300 mg, 0.622 mmol, 1.0 equiv) and benzo[d]thiazol-5-amine (112 mg, 0.75 mmol, 1.2 equiv) in EtOH (4 mL) was added TFA (0.2 mL) and the reaction mixture was stirred at 80° C. for 2 h. The progress of the reaction was monitored by TLC and LCMS. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to afford (1S,4S)-tert-butyl 5-(4-(4-(benzo[d]thiazol-5-ylamino)quinolin-7-yl)-3-fluorobenzoyl)-2,5-diazabicyclo[2.2.1]heptane-2-carboxylate (180 mg, 49%). LCMS: 596.2[M+1]⁺.

### Step-5: Synthesis of (4-(4-(benzo[d]thiazol-5-ylamino)quinolin-7-yl)-3-fluorophenyl)((1S,4S)-2,5-diazabicyclo[2.2.1]heptan-2-yl)methanone

To a solution of (1S,4S)-tert-butyl 5-(4-(4-(benzo[d]thiazol-5-ylamino)quinolin-7-yl)-3-fluorobenzoyl)-2,5-diazabicyclo[2.2.1]heptane-2-carboxylate (180 mg, 0.30 mmol, 1.0 equiv) in DCM (10 mL), was added TFA (1 mL) at 0° C. The reaction mixture was stirred at RT for 2 h. After completion of reaction, reaction mixture was concentrated under reduced pressure. The residue was basified with saturated sodium bicarbonate solution (50 mL), extracted with 10% MEOH/DCM (2×30 mL), dried over anhydrous sodium sulphate, filtered and concentrated under reduced pressure. The resulting solid was washed with diethyl ether and dried under vacuum to afford (4-(4-(benzo[d]thiazol-5-ylamino)quinolin-7-yl)-3-fluorophenyl)((1S,4S)-2,5-diazabicyclo[2.2.1]heptan-2-yl)methanone (30 mg, 20%), LCMS: 496.1 [M+1]^{+ 1}H NMR (400 MHz, DMSO-d₆) δ 9.43 (s, 1H), 9.28 (s, 1H), 8.54 (d, J=5.7 Hz, 2H), 8.21 (d, J=8.9 Hz, 1H), 8.05 (s, 1H), 8.10 (s, 1H), 7.86-7.69 (m, 2H), 7.59-7.35 (m, 3H), 7.06 (d, J=5.1 Hz, 1H), 4.67 (s, 1H), 4.30 (s, 1H), 3.72-3.54 (m, 1H), 3.49 (d, J=10.8 Hz, 1H), 3.05-2.91 (m, 1H), 2.87 (d, J=9.5 Hz, 1H), 1.84-1.69 (m, 1H), 1.66-1.51 (m, 1H).

### Example 102: Synthesis of (4-(4-(benzo[d]thiazol-5-ylamino)quinolin-7-yl)-3-fluorophenyl)(hexahydropyrrolo[3,4-c]pyrrol-2(1H)-yl)methanone (Compound 302)

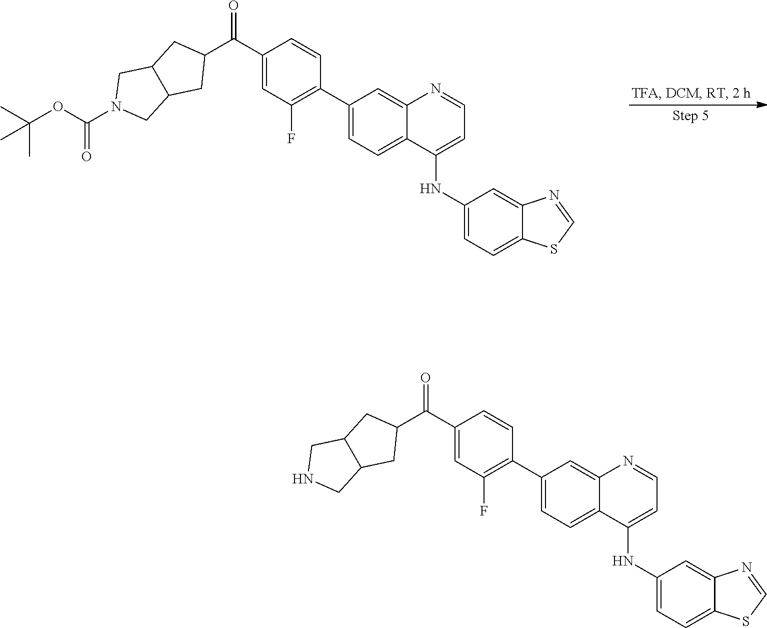 [SMILES: CC(C)(C)OC(=O)N1CC2CC(C(=O)c3ccc(-c4ccc5c(Nc6ccc7scnc7c6)ccnc5c4)c(F)c3)CC2C1 ; O=C(c1ccc(-c2ccc3c(Nc4ccc5scnc5c4)ccnc3c2)c(F)c1)C1CC2CNCC2C1]

To a solution tert-butyl 5-(4-(4-(benzo[d]thiazol-5-ylamino)quinolin-7-yl)-3-fluorobenzoyl)hexahydropyrrolo[3,4-c]pyrrole-2(1H)-carboxylate (50 mg, 0.08 mmol, 1.0 equiv) in dichloromethane (10 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-7-yl)-3-fluorophenyl)(hexahydropyrrolo[3,4-c]pyrrol-2(1H)-yl)methanone (12 mg, 32%), LCMS: 510.1 [M+1]⁺. ¹H NMR (400 MHz, DMSO-d₆) δ 2.88 (s, 3H) 3.64 (br. s., 2H) 3.89 (br. s., 2H) 4.02 (br. s., 1H) 4.18 (br. s., 1H) 6.96 (d, J=6.36 Hz, 1H) 7.49-7.58 (m, 2H) 7.63 (dd, J=8.27, 1.91 Hz, 1H) 7.85 (t, J=7.95 Hz, 1H) 8.12 (d, J=8.90 Hz, 1H) 8.22-8.31 (m, 1H) 8.41 (d, J=8.27 Hz, 1H) 8.58 (d, J=6.99 Hz, 1H) 8.99 (s, 1H) 9.54 (s, 1H) 11.10 (br. s., 1H) 14.29 (br. s., 1H).

### Example 103: Synthesis of (4-(4-(benzo[d]thiazol-5-ylamino)quinolin-7-yl)-2-fluorophenyl)(hexahydropyrrolo[3,4-c]pyrrol-2(1H)-yl)methanone (Compound 303)

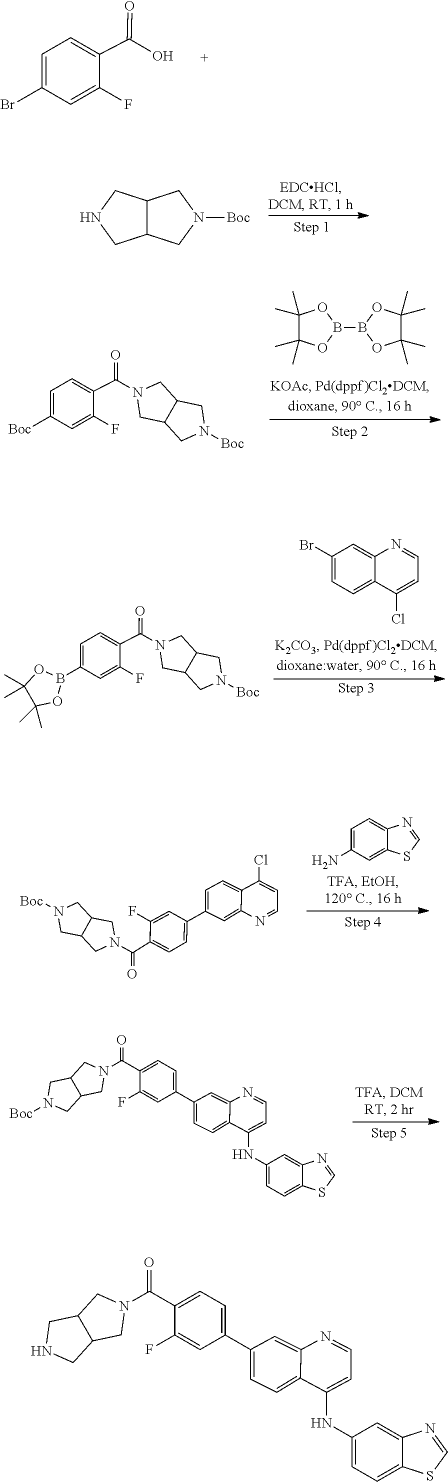

### Step-1: Synthesis of tert-butyl 5-(4-bromo-2-fluorobenzoyl)hexahydropyrrolo[3,4-c]pyrrole-2(1H)-carboxylate

To a solution 4-bromo-2-fluorobenzoic acid (500 mg, 2.88 mmol, 1.0 equiv) in DCM (15 mL) was added EDC·HCl (657 mg, 3.40 mmol, 1.3 equiv) and tert-butyl hexahydropyrrolo[3,4-c]pyrrole-2(1H)-carboxylate (484 mg, 2.88 mmol, 1.0 equiv). Reaction mixture was stirred at RT for 1 h. The progress of reaction was monitored by TLC. After completion of reaction, reaction mixture was diluted with water (30 mL) and extracted with DCM (2×60 ml), washed with brine solution, dried over anhydrous sodium sulphate, filtered and concentrated under reduced pressure to afford tert-butyl 5-(4-bromo-2-fluorobenzoyl)hexahydropyrrolo[3,4-c]pyrrole-2(1H)-carboxylate (900 mg, 95%), LCMS: 413 [M+1]⁺.

### Step-2: Synthesis of tert-butyl 5-(2-fluoro-4-(4,4,5,5-tetramethyl-1,3,2-dioxaborolan-2-yl)benzoyl)hexahydropyrrolo[3,4-c]pyrrole-2(1H)-carboxylate

To a solution tert-butyl 5-(4-bromo-2-fluorobenzoyl)hexahydropyrrolo[3,4-c]pyrrole-2(1H)-carboxylate (900 mg, 2.17 mmol, 1.0 equiv) in 1,4-dioxane (10 mL) was added KOAc (640 mg, 6.54 mmol, 3.0 equiv) and 4,4,4′,4′,5,5,5′,5′-octamethyl-2,2′-bi(1,3,2-dioxaborolane) (606 mg, 2.39 mmol, 1.1 equiv). Reaction mixture was purged with nitrogen for 5 min, then was added PdCl₂dppf·DCM (88 mg, 0.108 mmol, 0.05 equiv). The reaction was heated at 90° C. for 16 h. The progress of reaction was monitored by TLC. After completion of reaction, reaction mixture was diluted with water (60 mL) and extracted with ethyl acetate (2×120 mL), dried over anhydrous sodium sulphate, filtered and concentrated under reduced pressure. The crude product was purified by flash chromatography (0 to 20% EtOAc in hexane as an eluent) to afford crude which was purified by flash chromatography to afford tert-butyl 5-(2-fluoro-4-(4,4,5,5-tetramethyl-1,3,2-dioxaborolan-2-yl)benzoyl)hexahydropyrrolo[3,4-c]pyrrole-2(1H)-carboxylate (850 mg, 85%), LCMS: 461.1 [M+1]⁺.

### Step-3: Synthesis of tert-butyl 5-(4-(4-chloroquinolin-7-yl)-2-fluorobenzoyl)hexahydropyrrolo[3,4-c]pyrrole-2(1H)-carboxylate

To a solution of tert-butyl 5-(2-fluoro-4-(4,4,5,5-tetramethyl-1,3,2-dioxaborolan-2-yl)benzoyl)hexahydropyrrolo[3,4-c]pyrrole-2(1H)-carboxylate (650 mg, 2.6 mmol, 1.0 equiv) in 1,4-dioxane:water (8.5 ml:1.5 ml) was added K₂CO₃ (540 mg, 3.9 mmol, 1.5 equiv) and 7-bromo-4-chloroquinoline (1.4 gm, 3.2 mmol, 1.2 equiv). Reaction mixture was purged with nitrogen for 5 min, was added PdCl₂dppf·DCM (106 mg, 0.13 mmol, 0.05 equiv). The reaction mixture was heated at 90° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 20% EtOAc in hexane as an eluent) to afford tert-butyl 5-(4-(4-chloroquinolin-7-yl)-2-fluorobenzoyl)hexahydropyrrolo[3,4-c]pyrrole-2(1H)-carboxylate (250 mg, 35%), LCMS: 496.1 [M+1]⁺.

### Step-4: Synthesis of tert-butyl 5-(4-(4-(benzo[d]thiazol-5-ylamino)quinolin-7-yl)-2-fluorobenzoyl)hexahydropyrrolo[3,4-c]pyrrole-2(1H)-carboxylate

To a solution of tert-butyl 5-(4-(4-chloroquinolin-7-yl)-2-fluorobenzoyl)hexahydropyrrolo[3,4-c]pyrrole-2(1H)-carboxylate (400 mg, 0.80 mmol, 1.0 equiv) in ethanol (10 mL) was added benzo[d]thiazol-5-amine (121 mg, 0.80 mmol, 1.0 equiv) and TFA (0.051 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to afford tert-butyl 5-(4-(4-(benzo[d]thiazol-5-ylamino)quinolin-7-yl)-2-fluorobenzoyl)hexahydropyrrolo[3,4-c]pyrrole-2(1H)-carboxylate (100 mg, 20%), LCMS: 609[M+1]⁺.

### Step-5: Synthesis of (4-(4-(benzo[d]thiazol-5-ylamino)quinolin-7-yl)-2-fluorophenyl)(hexahydropyrrolo[3,4-c]pyrrol-2(1H)-yl)methanone

To a solution tert-butyl 5-(4-(4-(benzo[d]thiazol-5-ylamino)quinolin-7-yl)-2-fluorobenzoyl)hexahydropyrrolo[3,4-c]pyrrole-2(1H)-carboxylate (50 mg, 0.084 mmol, 1.0 equiv) in dichloromethane (10 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-7-yl)-2-fluorophenyl)(hexahydropyrrolo[3,4-c]pyrrol-2(1H)-yl)methanone (12 mg, 20%), LCMS: 510.1 [M+1]⁺. ¹H NMR (400 MHz, DMSO-d₆) δ 1.23 (s, 2H) 2.32 (br. s., 1H) 2.59-2.70 (m, 2H) 3.11 (d, J=6.99 Hz, 2H) 3.47-3.57 (m, 2H) 3.75-3.83 (m, 2H) 7.04 (d, J=5.72 Hz, 1H) 7.53-7.59 (m, 2H) 7.80-7.89 (m, 2H) 7.98 (d, J=8.90 Hz, 1H) 8.05 (s, 1H) 8.19-8.25 (m, 2H) 8.52-8.56 (m, 2H) 9.28 (s, 1H) 9.43 (s, 1H).

### Example 104: Synthesis of (4-(4-(benzo[d]thiazol-5-ylamino)quinolin-6-yl)-3-fluorophenyl)((1R,5S)-3,6-diazabicyclo[3.1.1]heptan-3-yl)methanone (Compound 304)

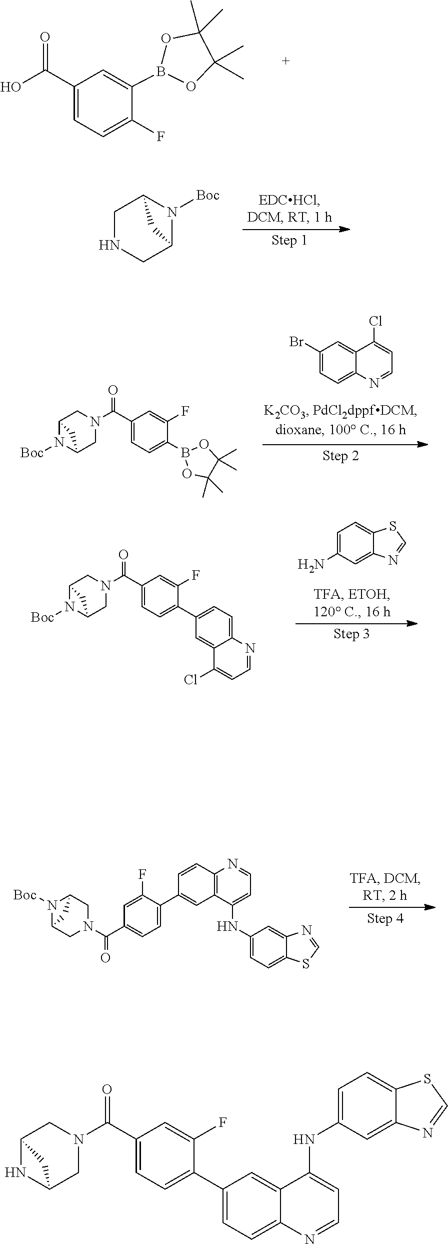

### Step-1: Synthesis of tert-butyl (1R,5S)-3-(3-fluoro-4-(4,4,5,5-tetramethyl-1,3,2-dioxaborolan-2-yl)benzoyl)-3,6-diazabicyclo[3.1.1]heptane-6-carboxylate

To a solution of 4-fluoro-3-(4,4,5,5-tetramethyl-1,3,2-dioxaborolan-2-yl)benzoic acid (350 mg, 1.31 mmol, 1.0 equiv) in DCM (15 mL) was added EDC·HCl (301 mg, 1.57 mmol, 1.2 equiv) and tert-butyl (1R,5S)-3,6-diazabicyclo[3.1.1]heptane-6-carboxylate (260 mg, 1.31 mmol, 1.0 equiv). Reaction was stirred at RT for 1 h. The progress of reaction was monitored by TLC. After completion of reaction, reaction mixture was diluted with water (30 mL) and extracted with DCM (2×60 ml), washed with brine solution, dried over anhydrous sodium sulphate, filtered and concentrated under reduced pressure. The crude product was purified by flash chromatography (0 to 20% EtOAc in hexane as an eluent) to afford tert-butyl (1R,5S)-3-(3-fluoro-4-(4,4,5,5-tetramethyl-1,3,2-dioxaborolan-2-yl)benzoyl)-3,6-diazabicyclo[3.1.1]heptane-6-carboxylate (100 mg, 17%), LCMS: 365.1[M+1]⁺[Boronic acid fragment]

### Step-2: Synthesis of tert-butyl (1R,5S)-3-(4-(4-chloroquinolin-6-yl)-3-fluorobenzoyl)-3,6-diazabicyclo[3.1.1]heptane-6-carboxylate

To a solution of tert-butyl (1R,5S)-3-(3-fluoro-4-(4,4,5,5-tetramethyl-1,3,2-dioxaborolan-2-yl)benzoyl)-3,6-diazabicyclo[3.1.1]heptane-6-carboxylate (100 mg, 0.224 mmol, 1.0 equiv) in 1,4-dioxane:water (8.5 ml:1.5 ml) was added K₂CO₃ (46 mg, 0.34 mmol, 1.5 equiv) and 6-bromo-4-chloroquinoline (65 mg, 0.27 mmol, 1.2 equiv). Reaction mixture was purged with nitrogen for 5 min, then was added PdCl₂dppf·DCM (9 mg, 0.011 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 20% EtOAc in hexane as an eluent) to afford tert-butyl (1R,5S)-3-(4-(4-chloroquinolin-6-yl)-3-fluorobenzoyl)-3,6-diazabicyclo[3.1.1]heptane-6-carboxylate (60 mg, 55%), LCMS: 482.1 [M+1]⁺.

### Step-3: Synthesis of tert-butyl (1R,5S)-3-(4-(4-(benzo[d]thiazol-5-ylamino)quinolin-6-yl)-3-fluorobenzoyl)-3,6-diazabicyclo[3.1.1]heptane-6-carboxylate

To a solution of tert-butyl (1R,5S)-3-(4-(4-chloroquinolin-6-yl)-3-fluorobenzoyl)-3,6-diazabicyclo[3.1.1]heptane-6-carboxylate (60 mg, 0.124 mmol, 1.0 equiv) in ethanol (10 mL) was added benzo[d]thiazol-5-amine (18 mg, 0.124 mmol, 1.0 equiv) and TFA (0.05 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to afford tert-butyl (1R,5S)-3-(4-(4-(benzo[d]thiazol-5-ylamino)quinolin-6-yl)-3-fluorobenzoyl)-3,6-diazabicyclo[3.1.1]heptane-6-carboxylate (40 mg, 54%), LCMS: 596.1 [M+1]⁺.

### Step-4: Synthesis of (4-(4-(benzo[d]thiazol-5-ylamino)quinolin-6-yl)-3-fluorophenyl)((1R,5S)-3,6-diazabicyclo[3.1.1]heptan-3-yl)methanone

To a solution of tert-butyl (1R,5S)-3-(4-(4-(benzo[d]thiazol-5-ylamino)quinolin-6-yl)-3-fluorobenzoyl)-3,6-diazabicyclo[3.1.1]heptane-6-carboxylate (40 mg, 0.07 mmol, 1.0 equiv) in dichloromethane (5 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-6-yl)-3-fluorophenyl)((1R,5S)-3,6-diazabicyclo[3.1.1]heptan-3-yl)methanone (10 mg, 30%), LCMS: 496.2[M+1]⁺. ¹H NMR (400 MHz, DMSO-d₆) δ 1.23 (s, 1H) 1.49 (d, J=8.27 Hz, 1H) 2.33 (br. s., 1H) 2.67 (br. s., 1H) 3.52 (d, J=11.44 Hz, 2H) 3.68 (d, J=12.72 Hz, 2H) 3.75-3.80 (m, 1H) 7.06 (d, J=5.72 Hz, 1H) 7.44-7.51 (m, 2H) 7.55 (s, 1H) 7.82 (t, J=7.95 Hz, 1H) 7.90-7.97 (m, 1H) 7.97-8.04 (m, 2H) 8.21 (d, J=8.27 Hz, 1H) 8.52 (d, J=5.09 Hz, 1H) 8.65 (s, 1H) 9.31 (s, 1H) 9.43 (s, 1H).

### Example 105: Synthesis of (4-(4-(benzo[d]thiazol-5-ylamino)quinolin-6-yl)-2-fluorophenyl)(hexahydropyrrolo[3,4-c]pyrrol-2(1H)-yl)methanone (Compound 305)

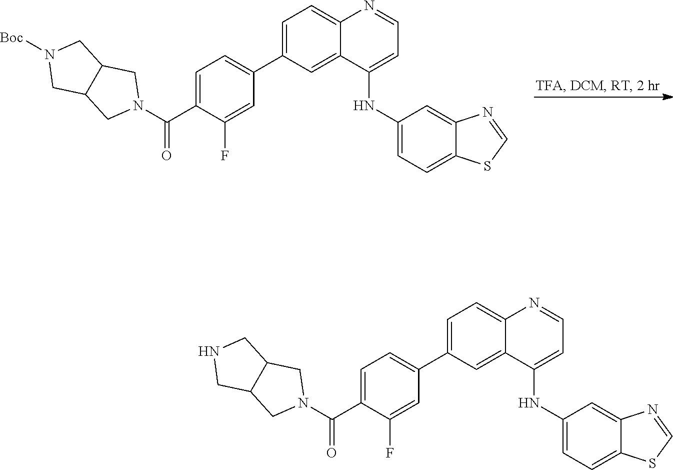 [SMILES: CC(C)(C)OC(=O)N1CC2CN(C(=O)c3ccc(-c4ccc5nccc(Nc6ccc7scnc7c6)c5c4)cc3F)CC2C1 ; O=C(c1ccc(-c2ccc3nccc(Nc4ccc5scnc5c4)c3c2)cc1F)N1CC2CNCC2C1]

To a solution of tert-butyl 5-(4-(4-(benzo[d]thiazol-5-ylamino)quinolin-6-yl)-2-fluorobenzoyl)hexahydropyrrolo[3,4-c]pyrrole-2(1H)-carboxylate (100 mg, 0.201 mmol, 1.0 equiv) in ethanol (5 mL) was added benzo[d]thiazol-5-amine (30 mg, 0.201 mmol, 1.0 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford crude which was purified by flash chromatography to afford (4-(4-(benzo[d]thiazol-5-ylamino)quinolin-6-yl)-2-fluorophenyl)(hexahydropyrrolo[3,4-c]pyrrol-2(1H)-yl)methanone (15 mg, 14%), LCMS: 510.3[M+1]⁺. ¹H NMR (400 MHz, DMSO-d₆) δ 1.23 (s, 2H) 2.32 (br. s., 1H) 2.59-2.70 (m, 2H) 3.11 (d, J=6.99 Hz, 2H) 3.47-3.57 (m, 2H) 3.75-3.83 (m, 2H) 7.04 (d, J=5.72 Hz, 1H) 7.53-7.59 (m, 2H) 7.80-7.89 (m, 2H) 7.98 (d, J=8.90 Hz, 1H) 8.05 (s, 1H) 8.19-8.25 (m, 2H) 8.52-8.56 (m, 2H) 9.28 (s, 1H) 9.43 (s, 1H).

### Example 106: Synthesis of (4-(4-(benzo[d]thiazol-5-ylamino)quinolin-6-yl)-3-fluorophenyl)((3aR,6aS)-hexahydropyrrolo[3,4-c]pyrrol-2(1H)-yl)methanone (Compound 306)

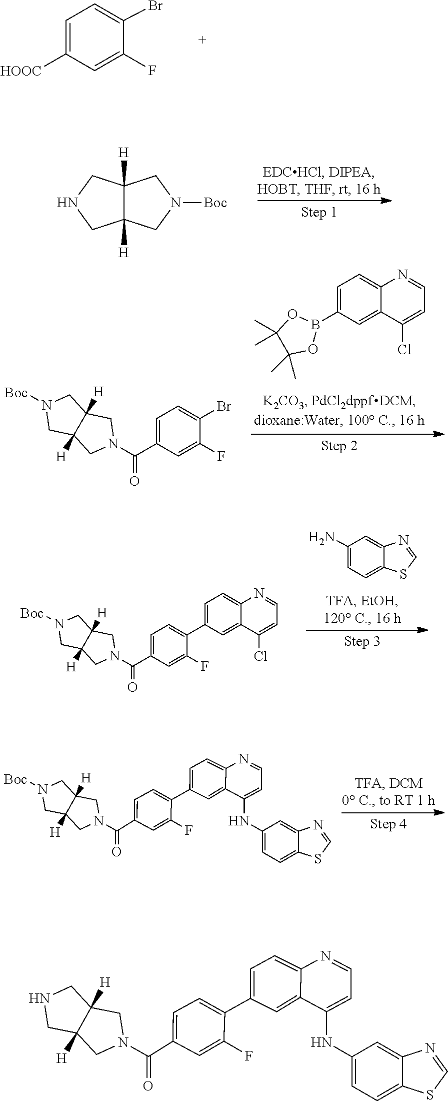

### Step-1: Synthesis of tert-butyl (3aR,6aS)-5-(4-bromo-3-fluorobenzoyl)hexahydropyrrolo[3,4-c]pyrrole-2(1H)-carboxylate

To a solution of 4-bromo-3-fluorobenzoic acid (400 mg, 1.83 mmol, 1.0 equiv) in THF (10 mL) was added EDC·HCl (455 mg, 2.374 mmol, 1.3 equiv) and HOBT (320 mg, 2.37 mmol, 1.3 equiv). Reaction was stirred at RT for 1 h. Then added, DIPEA (1 mL, 5.48 mmol, 3.0 equiv) and tert-butyl (3aR,6aS)-hexahydropyrrolo[3,4-c]pyrrole-2(1H)-carboxylate (465 mg, 2.19 mmol, 1.2 equiv). Reaction was stirred at RT for 16 h. The progress of reaction was monitored by TLC. After completion of reaction, reaction mixture was diluted with water (40 mL) and extracted with ethyl acetate (2×80 mL), washed with brine solution, dried over anhydrous sodium sulphate, filtered and concentrated under reduced pressure. The crude product was purified by flash chromatography (0 to 20% EtOAc in hexane as an eluent) to afford tert-butyl (3aR,6aS)-5-(4-bromo-3-fluorobenzoyl)hexahydropyrrolo[3,4-c]pyrrole-2(1H)-carboxylate (680 mg, 90%), LCMS: 413.1 [M+1]⁺.

### Step-2: Synthesis of tert-butyl (3aR,6aS)-5-(4-(4-chloroquinolin-6-yl)-3-fluorobenzoyl)hexahydropyrrolo[3,4-c]pyrrole-2(1H)-carboxylate

To a solution of tert-butyl (3aR,6aS)-5-(4-bromo-3-fluorobenzoyl)hexahydropyrrolo[3,4-c]pyrrole-2(1H)-carboxylate (300 mg, 0.68 mmol, 1.0 equiv) in 1,4-dioxane:water (6 mL:2 mL) was added K₂CO₃ (187 mg, 1.36 mmol, 2.0 equiv) and 4-chloro-6-(4,4,5,5-tetramethyl-1,3,2-dioxaborolan-2-yl)quinoline (196 mg, 0.68 mmol, 1.0 equiv). Reaction mixture was purged with nitrogen for 5 min, then was added PdCl₂dppf·DCM (27 mg, 0.033 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 20% EtOAc in hexane as an eluent) to afford tert-butyl (3aR,6aS)-5-(4-(4-chloroquinolin-6-yl)-3-fluorobenzoyl)hexahydropyrrolo[3,4-c]pyrrole-2(1H)-carboxylate (330 mg, 91%), LCMS: 496.1 [M+1]⁺.

### Step-3: Synthesis of tert-butyl (3aR,6aS)-5-(4-(4-(benzo[d]thiazol-5-ylamino)quinolin-6-yl)-3-fluorobenzoyl)hexahydropyrrolo[3,4-c]pyrrole-2(1H)-carboxylate

To a solution of tert-butyl (3aR,6aS)-5-(4-(4-chloroquinolin-6-yl)-3-fluorobenzoyl)hexahydropyrrolo[3,4-c]pyrrole-2(1H)-carboxylate (330 mg, 0.66 mmol, 1.0 equiv) in ethanol (5 mL) was added benzo[d]thiazol-5-amine (149 mg, 0.99 mmol, 1.5 equiv) and TFA (0.2 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×80 mL), dried over anhydrous sodium sulphate, filtered and concentrated under reduced pressure. The crude product was purified by flash column chromatography (0 to 6% MeOH in DCM as an eluent) to afford tert-butyl (3aR,6aS)-5-(4-(4-(benzo[d]thiazol-5-ylamino)quinolin-6-yl)-3-fluorobenzoyl)hexahydropyrrolo[3,4-c]pyrrole-2(1H)-carboxylate (150 mg, 37%), LCMS: 610.2 [M+1]⁺.

### Step-4: Synthesis of (4-(4-(benzo[d]thiazol-5-ylamino)quinolin-6-yl)-3-fluorophenyl)((3aR,6aS)-hexahydropyrrolo[3,4-c]pyrrol-2(1H)-yl)methanone

To a solution of tert-butyl (3aR,6aS)-5-(4-(4-(benzo[d]thiazol-5-ylamino)quinolin-6-yl)-3-fluorobenzoyl)hexahydropyrrolo[3,4-c]pyrrole-2(1H)-carboxylate (150 mg, 0.25 mmol, 1.0 equiv) in DCM (5 mL) was added TFA (1 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-6-yl)-3-fluorophenyl)((3aR,6aS)-hexahydropyrrolo[3,4-c]pyrrol-2(1H)-yl)methanone (70 mg, 56%), LCMS: 510.1[M+1]^{+ 1}H NMR (400 MHz, DMSO-d₆) δ 1.21 (br. s., 1H) 3.06 (br. s., 4H) 3.15 (br. s., 2H) 3.37 (br. s., 2H) 3.43 (br. s., 2H) 6.96 (d, J=6.36 Hz, 1H) 7.51-7.65 (m, 3H) 7.82 (t, J=7.63 Hz, 1H) 8.09 (d, J=8.90 Hz, 1H) 8.20-8.27 (m, 3H) 8.38 (d, J=8.27 Hz, 1H) 8.53 (d, J=6.99 Hz, 1H) 8.95 (s, 1H) 9.50 (s, 1H).

### Example 107: Synthesis of (4-(4-(benzo[d]thiazol-5-ylamino)quinolin-7-yl)-3-fluorophenyl)((3aR,6aS)-hexahydropyrrolo[3,4-c]pyrrol-2(1H)-yl)methanone (Compound 307)

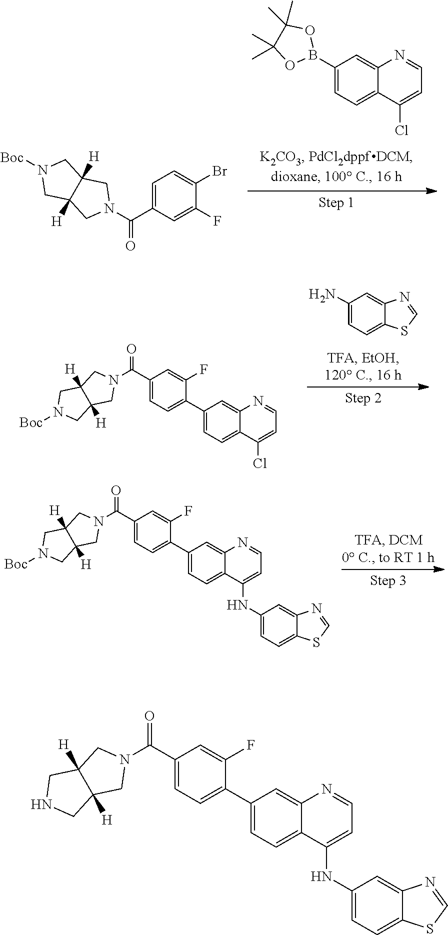 [SMILES: CC1(C)OB(c2ccc3c(Cl)ccnc3c2)OC1(C)C ; CC(C)(C)OC(=O)N1C[C@@H]2CN(C(=O)c3ccc(Br)c(F)c3)C[C@@H]2C1 ; Nc1ccc2scnc2c1 ; CC(C)(C)OC(=O)N1C[C@@H]2CN(C(=O)c3ccc(-c4ccc5c(Cl)ccnc5c4)c(F)c3)C[C@@H]2C1 ; CC(C)(C)OC(=O)N1C[C@@H]2CN(C(=O)c3ccc(-c4ccc5c(Nc6ccc7scnc7c6)ccnc5c4)c(F)c3)C[C@@H]2C1 ; O=C(c1ccc(-c2ccc3c(Nc4ccc5scnc5c4)ccnc3c2)c(F)c1)N1C[C@H]2CNC[C@H]2C1]

### Step-1: Synthesis of tert-butyl (3aR,6aS)-5-(4-(4-chloroquinolin-7-yl)-3-fluorobenzoyl)hexahydropyrrolo[3,4-c]pyrrole-2(1H)-carboxylate

To a solution of tert-butyl (3aR,6aS)-5-(4-bromo-3-fluorobenzoyl)hexahydropyrrolo[3,4-c]pyrrole-2(1H)-carboxylate (300 mg, 0.68 mmol, 1.0 equiv) in 1,4-dioxane:water (6 mL:2 mL) was added K₂CO₃ (187 mg, 1.36 mmol, 2 equiv) and 4-chloro-7-(4,4,5,5-tetramethyl-1,3,2-dioxaborolan-2-yl)quinoline (196 mg, 0.68 mmol, 1.0 equiv). Reaction mixture was purged with nitrogen for 5 min, then was added PdCl₂dppf·DCM (27 mg, 0.033 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 20% EtOAc in hexane as an eluent) to afford crude which was purified by flash chromatography to afford tert-butyl (3aR,6aS)-5-(4-(4-chloroquinolin-7-yl)-3-fluorobenzoyl)hexahydropyrrolo[3,4-c]pyrrole-2(1H)-carboxylate (157 mg, 43%), LCMS: 496.1[M+1]⁺.

### Step-2: Synthesis of tert-butyl (3aR,6aS)-5-(4-(4-(benzo[d]thiazol-5-ylamino)quinolin-7-yl)-3-fluorobenzoyl)hexahydropyrrolo[3,4-c]pyrrole-2(1H)-carboxylate

To a solution of tert-butyl (3aR,6aS)-5-(4-(4-chloroquinolin-7-yl)-3-fluorobenzoyl)hexahydropyrrolo[3,4-c]pyrrole-2(1H)-carboxylate (157 mg, 0.32 mmol, 1.0 equiv) in ethanol (5 mL) was added benzo[d]thiazol-5-amine (71 mg, 0.47 mmol, 1.5 equiv) and TFA (0.15 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×80 mL), dried over anhydrous sodium sulphate, filtered and concentrated under reduced pressure. The crude product was purified by flash column chromatography (0 to 6% MeOH in DCM as an eluent) to afford tert-butyl (3aR,6aS)-5-(4-(4-(benzo[d]thiazol-5-ylamino)quinolin-7-yl)-3-fluorobenzoyl)hexahydropyrrolo[3,4-c]pyrrole-2(1H)-carboxylate (110 mg, 57%), LCMS: 610.2[M+1]⁺.

### Step-3: Synthesis of (4-(4-(benzo[d]thiazol-5-ylamino)quinolin-7-yl)-3-fluorophenyl)((3aR,6aS)-hexahydropyrrolo[3,4-c]pyrrol-2(1H)-yl)methanone

To a solution of tert-butyl (3aR,6aS)-5-(4-(4-(benzo[d]thiazol-5-ylamino)quinolin-6-yl)-3-fluorobenzoyl)hexahydropyrrolo[3,4-c]pyrrole-2(1H)-carboxylate (110 mg, 0.18 mmol, 1.0 equiv) in DCM (5 mL) was added TFA (1 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-7-yl)-3-fluorophenyl)((3aR,6aS)-hexahydropyrrolo[3,4-c]pyrrol-2(1H)-yl)methanone (70 mg, 77%), LCMS: 510.1[M+1]^{+ 1}H NMR (400 MHz, DMSO-d₆) δ 1.21 (br. s., 1H) 3.06 (br. s., 4H) 3.15 (br. s., 2H) 3.37 (br. s., 2H) 3.43 (br. s., 2H) 6.96 (d, J=6.36 Hz, 1H) 7.51-7.65 (m, 3H) 7.82 (t, J=7.63 Hz, 1H) 8.09 (d, J=8.90 Hz, 1H) 8.20-8.27 (m, 3H) 8.38 (d, J=8.27 Hz, 1H) 8.53 (d, J=6.99 Hz, 1H) 8.95 (s, 1H) 9.50 (s, 1H) Example 108: Synthesis of (4-(4-(benzo[d]thiazol-5-ylamino)quinolin-6-yl)-3-fluorophenyl)((1R,5S)-3,6-diazabicyclo[3.1.1]heptan-6-yl)methanone (Compound 308)

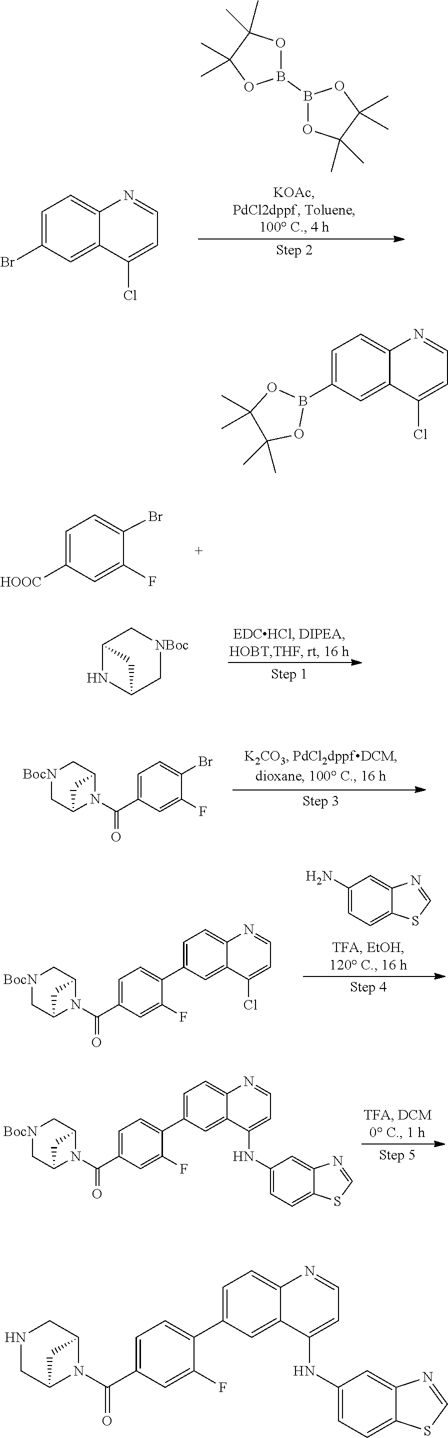

### Step-1: Synthesis of tert-butyl (1R,5S)-6-(4-bromo-3-fluorobenzoyl)-3,6-diazabicyclo[3.1.1]heptane-3-carboxylate

To a solution of 4-bromo-3-fluorobenzoic acid (400 mg, 1.83 mmol, 1.0 equiv) in THF (10 mL) was added EDC·HCl (455 mg, 2.37 mmol, 1.3 equiv) and HOBT (320 mg, 2.37 mmol, 1.3 equiv). Reaction was stirred at RT for 1 h. Then added, DIPEA (1 mL, 5.48 mmol, 3 equiv) and tert-butyl (1R,5S)-3,6-diazabicyclo[3.1.1]heptane-3-carboxylate (434 mg, 2.19 mmol, 1.2 equiv). Reaction was stirred at RT for 16 h. The progress of reaction was monitored by TLC. After completion of reaction, reaction mixture was diluted with water (40 mL) and extracted with ethyl acetate (2×80 mL), washed with brine solution, dried over anhydrous sodium sulphate, filtered and concentrated under reduced pressure. The crude product was purified by flash chromatography (0-50% ethyl acetate:hexane as an eluent) to afford tert-butyl (1R,5S)-6-(4-bromo-3-fluorobenzoyl)-3,6-diazabicyclo[13.1.1]heptane-3-carboxylate (700 mg, 96%), LCMS: 343.1[acid fragment]

### Step-2: Synthesis of 4-chloro-6-(4,4,5,5-tetramethyl-1,3,2-dioxaborolan-2-yl)quinoline

To a solution of 6-bromo-4-chloroquinoline (200 mg, 0.82 mmol, 1.0 equiv) in 1,4-dioxane (5 mL) was added KOAc (242 mg, 2.47 mmol, 3.0 equiv) and 4,4,4′,4′,5,5,5′,5′-octamethyl-2,2′-bi(1,3,2-dioxaborolane) (314 mg, 1.24 mmol, 1.5 equiv). Reaction mixture was purged with nitrogen for 5 min, then was added PdCl2dppf (30 mg, 0.041 mmol, 0.05 equiv). The reaction was heated at 100° C. for 4 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure to afford 4-chloro-6-(4,4,5,5-tetramethyl-1,3,2-dioxaborolan-2-yl)quinoline (210 mg, 88%), LCMS: 208.2 [boronic acid fragment]

### Step-3: Synthesis of tert-butyl (1R,5S)-6-(4-(4-chloroquinolin-6-yl)-3-fluorobenzoyl)-3,6-diazabicyclo[3.1.1]heptane-3-carboxylate

To a solution of tert-butyl (1R,5S)-6-(4-bromo-3-fluorobenzoyl)-3,6-diazabicyclo[3.1.1]heptane-3-carboxylate (250 mg, 0.63 mmol, 1.0 equiv) in 1,4-dioxane:water (6 mL:2 mL) was added K₂CO₃ (173 mg, 1.25 mmol, 2.0 equiv) and 4-chloro-6-(4,4,5,5-tetramethyl-1,3,2-dioxaborolan-2-yl)quinoline (181 mg, 0.63 mmol, 1.0 equiv). Reaction mixture was purged with nitrogen for 5 min, then was added PdCl₂dppf·DCM (25 mg, 0.03 mmol, 0.05 equiv). The reaction was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure. The crude product was purified by flash chromatography (0-2% MeOH:DCM as an eluent) to afford tert-butyl (1R,5S)-6-(4-(4-chloroquinolin-6-yl)-3-fluorobenzoyl)-3,6-diazabicyclo[3.1.1]heptane-3-carboxylate (130 mg, 43%), LCMS: 482.1[M+1]⁺.

### Step-4: Synthesis of tert-butyl (1R,5S)-6-(4-(4-(benzo[d]thiazol-5-ylamino)quinolin-6-yl)-3-fluorobenzoyl)-3,6-diazabicyclo[3.1.1]heptane-3-carboxylate

To a solution of tert-butyl (1R,5S)-6-(4-(4-chloroquinolin-6-yl)-3-fluorobenzoyl)-3,6-diazabicyclo[3.1.1]heptane-3-carboxylate (130 mg, 0.27 mmol, 1.0 equiv) in ethanol (5 mL) was added benzo[d]thiazol-5-amine (60 mg, 0.40 mmol, 1.5 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×50 mL), dried over anhydrous sodium sulphate, filtered and concentrated under reduced pressure. The crude product was purified by flash column chromatography (0 to 5% MeOH in DCM as an eluent) to afford tert-butyl (1R,5S)-6-(4-(4-(benzo[d]thiazol-5-ylamino)quinolin-6-yl)-3-fluorobenzoyl)-3,6-diazabicyclo[3.1.1]heptane-3-carboxylate (90 mg, 56%), LCMS: 596.4[M+1]⁺.

### Step-5: Synthesis of (4-(4-(benzo[d]thiazol-5-ylamino)quinolin-6-yl)-3-fluorophenyl)((1R,5S)-3,6-diazabicyclo[3.1.1]heptan-6-yl)methanone

To a solution of tert-butyl (1R,5S)-6-(4-(4-chloroquinolin-6-yl)-3-fluorobenzoyl)-3,6-diazabicyclo[3.1.1]heptane-3-carboxylate (90 mg, 0.15 mmol, 1.0 equiv) in DCM (5 mL) was added TFA (1 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with DCM (2×40 mL), dried over anhydrous sodium sulphate, filtered and concentrated under reduced pressure. The crude product was purified by flash column chromatography (0 to 8% MeOH in DCM) to afford (4-(4-(benzo[d]thiazol-5-ylamino)quinolin-6-yl)-3-fluorophenyl)((1R,5S)-3,6-diazabicyclo[3.1.1]heptan-6-yl)methanone (72 mg, 97%), LCMS: 496.3[M+1]^{+ 1}H NMR (400 MHz, DMSO-d₆) δ 9.54 (br. s., 1H) 9.17 (br. s., 1H) 9.00 (br. s., 1H) 8.58 (br. s., 1H) 8.41 (d, J=6.99 Hz, 1H) 8.24 (s, 1H) 8.26 (s, 1H) 8.14 (br. s., 1H) 7.88 (br. s., 1H) 7.73 (d, J=8.27 Hz, 2H) 7.63 (br. s., 1H) 6.97 (br. s., 1H) 4.54 (s, 2H) 3.63 (br. s., 2H) 3.39 (br. s., 1H) 2.91 (br. s., 1H) 2.33 (br. s., 1H) 1.90 (d, J=8.90 Hz, 1H).

### Example 109: Synthesis of N-(7-(2-fluoro-4-(piperidin-4-yloxy)phenyl)quinolin-4-yl)benzo[d]thiazol-5-amine (Compound 309)

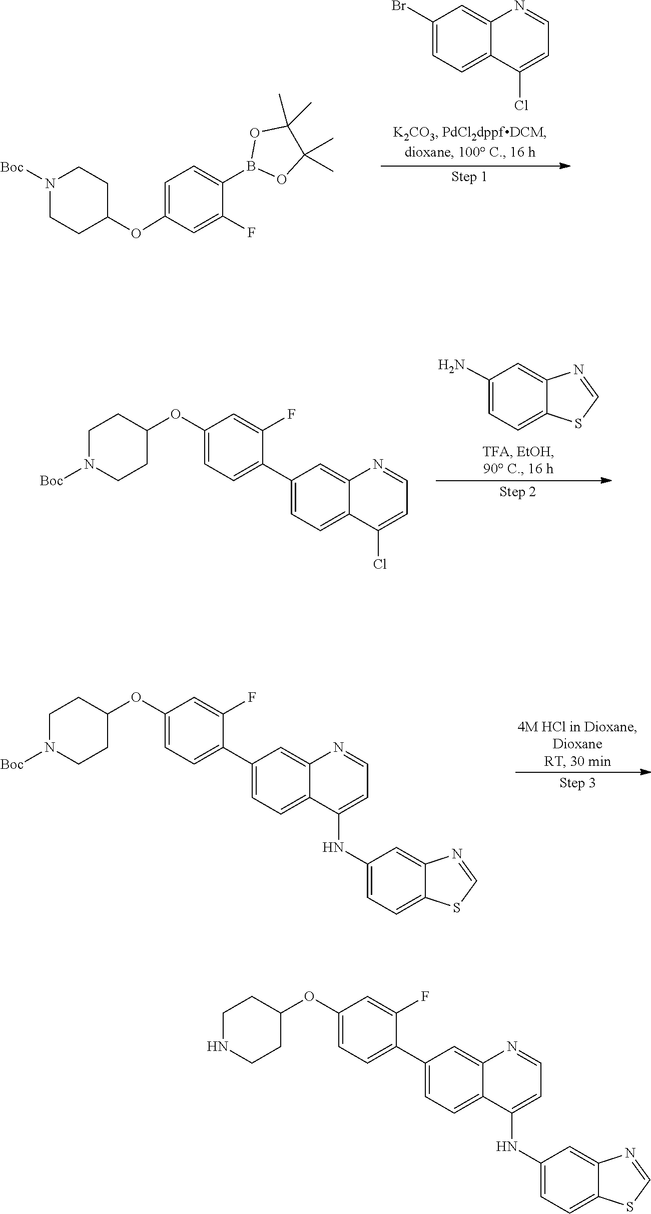 [SMILES: CC(C)(C)OC(=O)N1CCC(Oc2ccc(B3OC(C)(C)C(C)(C)O3)c(F)c2)CC1 ; Clc1ccnc2cc(Br)ccc12 ; CC(C)(C)OC(=O)N1CCC(Oc2ccc(-c3ccc4c(Cl)ccnc4c3)c(F)c2)CC1 ; Nc1ccc2scnc2c1 ; CC(C)(C)OC(=O)N1CCC(Oc2ccc(-c3ccc4c(Nc5ccc6scnc6c5)ccnc4c3)c(F)c2)CC1 ; Fc1cc(OC2CCNCC2)ccc1-c1ccc2c(Nc3ccc4scnc4c3)ccnc2c1]

### Step-1: Synthesis of tert-butyl 4-(4-(4-chloroquinolin-7-yl)-3-fluorophenoxy)piperidine-1-carboxylate

To a stirred solution of 7-bromo-4-chloroquinoline (200 mg, 0.82 mmol, 1.0 equiv) and tert-butyl 4-(3-fluoro-4-(4,4,5,5-tetramethyl-1,3,2-dioxaborolan-2-yl)phenoxy)piperidine-1-carboxylate (416.9 mg, 0.99 mmol, 1.2 equiv) in 1,4-dioxane:water (4 mL:0.5 mL) was added K₂CO₃ (227.5 mg, 1.648 mmol, 2.0 equiv). Reaction mixture was purged with N2 gas for 10 min and was added Pd(dppf)Cl2·DCM (33.7 mg, 0.041 mmol, 0.05 equiv). Reaction mixture was stirred at 100° C. for 16 h. The progress of the reaction was monitored by LCMS and TLC. After completion of reaction, reaction mixture was diluted with water (75 mL) and was extracted with EtOAc (2×100 mL), combined organic layers were dried over Na₂SO₄, filtered and concentrated under reduced pressure to afford crude which was purified by flash chromatography (eluted in 2% MeOH:DCM) to afford tert-butyl 4-(4-(4-chloroquinolin-7-yl)-3-fluorophenoxy)piperidine-1-carboxylate. (250 mg, 66%), LCMS: 457.2M+1]⁺.

### Step-2: Synthesis of tert-butyl 4-(4-(4-(benzo[d]thiazol-5-ylamino)quinolin-7-yl)-3-fluorophenoxy)piperidine-1-carboxylate

To a solution of tert-butyl 4-(4-(4-chloroquinolin-7-yl)-3-fluorophenoxy)piperidine-1-carboxylate (70 mg, 0.15 mmol, 1.0 equiv) and benzo[d]thiazol-5-amine (23 mg, 0.15 mmol, 1.0 equiv) in EtOH (3 mL) was added TFA (0.05 mL) and the reaction mixture was stirred at 90° C. for 16 h. The progress of reaction was monitored by TLC and LCMS. After completion of reaction, reaction mixture was concentrated under reduced pressure. The resulting residue was basified with saturated sodium bicarbonate solution (20 mL), was extracted with ethyl acetate (2×80 mL), dried over anhydrous sodium sulphate, filtered and concentrated under reduced pressure. The crude product was purified by flash column chromatography (0 to 6% MeOH in DCM as an eluent) to afford tert-butyl 4-(4-(4-(benzol[d]thiazol-5-ylamino)quinolin-7-yl)-3-fluorophenoxy)piperidine-1-carboxylate (40 mg, 46%), LCMS: 571.2 [M+1]⁺.

### Step-3: Synthesis of N-(7-(2-fluoro-4-(piperidin-4-yloxy)phenyl)quinolin-4-yl)benzo[d]thiazol-5-amine

To a solution of tert-butyl 4-(4-(4-(benzo[d]thiazol-5-ylamino)quinolin-7-yl)-3-fluorophenoxy)piperidine-1-carboxylate (30 mg, 0.12 mmol, 1.0 equiv) in DCM (4 mL), we added TFA (0.2 mL) at 0° C. The reaction mixture was stirred at RT for 30 min. The progress of reaction was monitored by TLC and LCMS. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by reversed phase HPLC to afford N-(7-(2-fluoro-4-(piperidin-4-yloxy)phenyl)quinolin-4-yl)benzo[d]thiazol-5-amine (5 mg, 20%), LCMS: 471.2 [M+1]⁺, ¹H NMR (400 MHz, DMSO-d₆) δ 9.43 (s, 1H) 8.96 (s, 1H) 8.80 (s, 1H) 8.46-8.52 (m, 2H) 8.20 (d, J=8.90 Hz, 1H) 8.01 (s, 2H) 7.69-7.74 (m, 1H) 7.64-7.69 (m, 1H) 7.55 (d, J=8.27 Hz, 1H) 7.11 (d, J=12.72 Hz, 1H) 7.02 (d, J=5.72 Hz, 2H) 4.74 (br. s., 1H) 3.16 (br. s., 2H) 3.05 (br. s., 2H) 2.06 (br. s., 2H) 1.82 (br. s., 2H).

### Example 110: Synthesis of (3-aminoazetidin-1-yl)(4-(4-(benzo[d]thiazol-5-ylamino)quinolin-7-yl)-3-fluorophenyl)methanone (Compound 310)

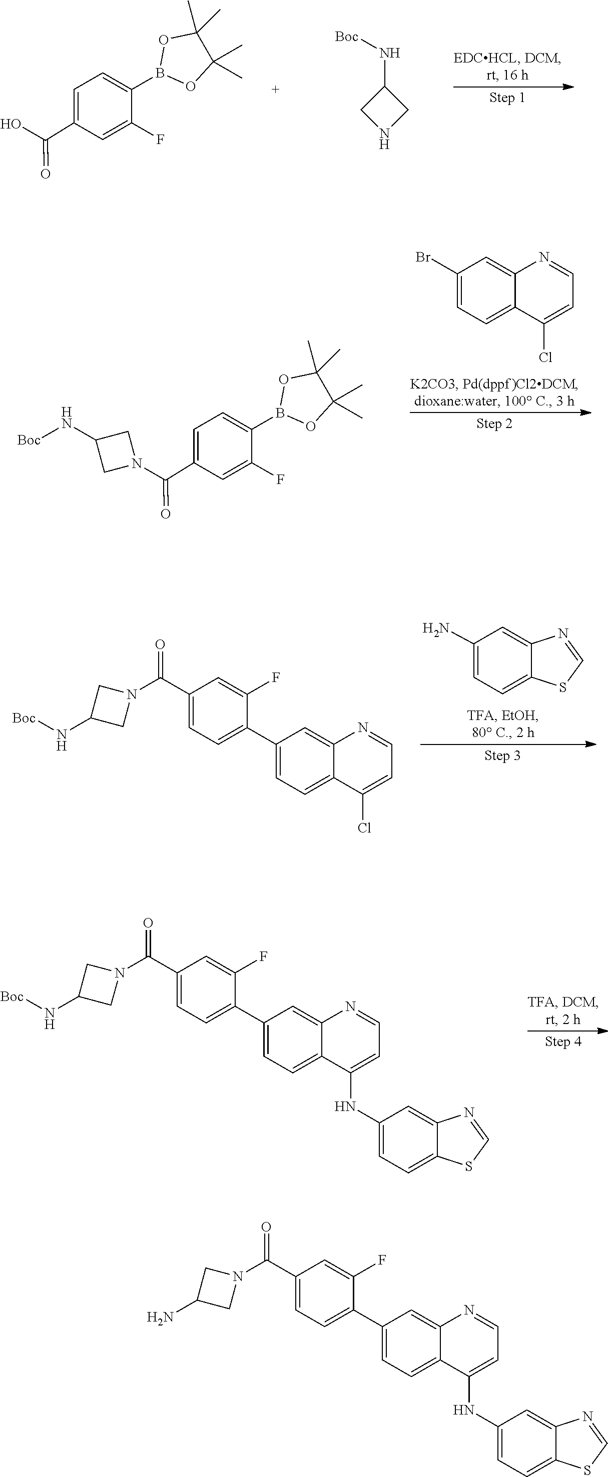

### Step-1: Synthesis of tert-butyl 1-(3-fluoro-4-(4,4,5,5-tetramethyl-1,3,2-dioxaborolan-2-yl)benzoyl)azetidin-3-ylcarbamate

To a solution 3-fluoro-4-(4,4,5,5-tetramethyl-1,3,2-dioxaborolan-2-yl)benzoic acid (500 mg, 1.87 mmol, 1.0 equiv) in DCM (10 mL) was added EDC·HCl (466 mg, 2.43 mmol, 1.3 equiv) and tert-butyl azetidin-3-ylcarbamate (388 mg, 2.25 mmol, 1.2equiv). Reaction was stirred at RT for 16 h. The progress of reaction was monitored by TLC. After completion of reaction, reaction mixture was diluted with water (40 mL) and extracted with DCM (2×80 ml), washed with brine solution, dried over anhydrous sodium sulphate, filtered and concentrated under reduced pressure to afford tert-butyl 1-(3-fluoro-4-(4,4,5,5-tetramethyl-1,3,2-dioxaborolan-2-yl)benzoyl)azetidin-3-ylcarbamate (600 mg, 76%), LCMS: 283.2 [M+1]⁺ (Boronic acid fragment)

### Step-2: Synthesis of tert-butyl 1-(4-(4-chloroquinolin-7-yl)-3-fluorobenzoyl)azetidin-3-ylcarbamate

To a solution of tert-butyl 1-(3-fluoro-4-(4,4,5,5-tetramethyl-1,3,2-dioxaborolan-2-yl)benzoyl)azetidin-3-ylcarbamate (600 mg, 1.42 mmol, 1.0 equiv) in 1,4-dioxane:water (8.5 ml:1.5 ml) was added K₂CO₃ (490 mg, 3.55 mmol, 2.5 equiv) and 7-bromo-4-chloroquinoline (415 mg, 1.71 mmol, 1.2 equiv). Reaction mixture was purged with nitrogen for 5 min, was added PdCl₂dppf·DCM (58 mg, 0.07 mmol, 0.05 equiv). The reaction mixture was heated at 100° C. for 3 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure. The crude product was purified by flash chromatography (0-2% MeOH:DCM as an eluent) to afford tert-butyl 1-(4-(4-chloroquinolin-7-yl)-3-fluorobenzoyl)azetidin-3-ylcarbamate (350 mg, 53%), LCMS: 456.2 M+1]⁺.

### Step-3: Synthesis of tert-butyl (1-(4-(4-(benzo[d]thiazol-5-ylamino)quinolin-7-yl)-3-fluorobenzoyl)azetidin-3-yl)carbamate

To a solution of tert-butyl 1-(4-(4-chloroquinolin-7-yl)-3-fluorobenzoyl)azetidin-3-ylcarbamate (350 mg, 0.76 mmol, 1.0 equiv) in ethanol (10 mL) was added benzo[d]thiazol-5-amine (138 mg, 0.92 mmol, 1.2 equiv) and TFA (0.4 mL). The reaction mixture was heated at 80° C. for 2 h. After completion of reaction, reaction mixture was concentrated under reduced pressure. The resulting residue was basified with saturated sodium bicarbonate solution (20 mL), was extracted with ethyl acetate (2×50 mL), dried over anhydrous sodium sulphate, filtered and concentrated under reduced pressure. The crude product was purified by flash column chromatography (0 to 5% MeOH in DCM as an eluent) to afford tert-butyl (1-(4-(4-(benzo[d]thiazol-5-ylamino)quinolin-7-yl)-3-fluorobenzoyl)azetidin-3-yl)carbamate (100 mg, 27%), LCMS: 570.1[M+1]⁺.

### Step-4: Synthesis of (3-aminoazetidin-1-yl)(4-(4-(benzo[d]thiazol-5-ylamino)quinolin-7-yl)-3-fluorophenyl)methanone

To a solution of tert-butyl (1-(4-(4-(benzo[d]thiazol-5-ylamino)quinolin-7-yl)-3-fluorobenzoyl)azetidin-3-yl)carbamate (100 mg, 0.17 mmol, 1 equiv)) in dichloromethane (10 mL) was added TFA (1 mL) at 0° C. The reaction mixture was stirred at RT for 2 h. After completion of reaction, reaction mixture was concentrated under reduced pressure. The resulting residue was basified with saturated sodium bicarbonate solution (10 mL), extracted with DCM (2×20 mL), dried over anhydrous sodium sulphate, filtered and concentrated under reduced pressure. The resulting solid was washed with diethyl ether and dried under vacuum to afford (3-aminoazetidin-1-yl)(4-(4-(benzo[d]thiazol-5-ylamino)quinolin-7-yl)-3-fluorophenyl)methanone (20 mg, 24%). LCMS: 470.2[M+1]^{+ 1}H NMR (400 MHz, DMSO-d₆) δ 9.43 (s, 1H), 9.28 (br. s., 1H), 8.54 (d, J=8.9 Hz, 2H), 8.21 (d, J=8.3 Hz, 1H), 8.10 (s, 1H), 8.05 (s, 1H), 7.85-7.73 (m, 2H), 7.64-7.49 (m, 3H), 7.05 (d, J=5.1 Hz, 1H), 4.54 (d, J=6.4 Hz, 1H), 4.24 (d, J=6.4 Hz, 1H), 4.03 (s, 2H), 3.77 (br. s., 2H), 3.17 (d, J=5.1 Hz, 1H).

### Example 111: Synthesis of N-(6-(4-(2-azaspiro[3.3]heptan-6-yloxy)-2-fluorophenyl)quinolin-4-yl)benzo[d]thiazol-5-amine (Compound 311)

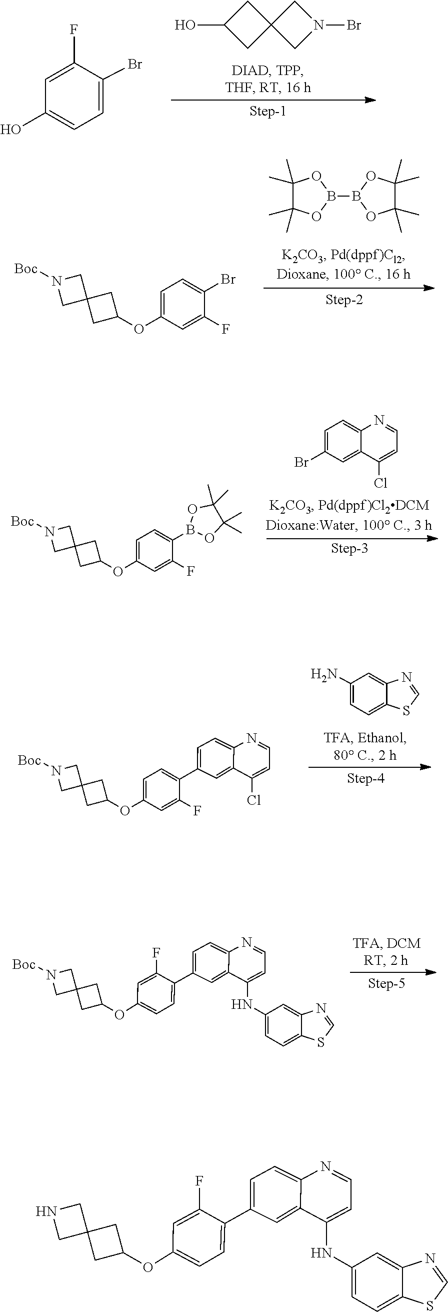

### Step-1: Synthesis of tert-butyl 6-(4-bromo-3-fluorophenoxy)-2-azaspiro[3.3]heptane-2-carboxylate

To a stirred solution of TPP (2.05 g, 7.84 mmol, 1.5 equiv) in THF (10 mL), was added DIAD (1.58 g, 7.84 mmol, 1.5 equiv) dropwise at 0° C. After precipitation, 4-bromo-3-fluorophenol (1 g, 5.23 mmol, 1.0 equiv) in THF (7 mL) and tert-butyl 6-hydroxy-2-azaspiro[3.3]heptane-2-carboxylate (1.34 g, 6.28 mmol, 1.2 equiv) were added. Resulting reaction mixture was stirred at RT for 16 h. The progress of the reaction was monitored by TLC and LCMS. Upon completion of reaction, reaction mixture was concentrated under reduced pressure. The resulting residue was suspended in hexane (200 mL), resulting white solid was filtered off. Filtrate was collected, diluted with ethyl acetate and washed with water (100 mL). Organic layer was dried over anhydrous Na₂SO₄, filtered and concentrated under reduced pressure. The crude product was purified by flash chromatography (0 to 20% EtOAc in hexane as an eluent) to afford tert-butyl 6-(4-bromo-3-fluorophenoxy)-2-azaspiro[3.3]heptane-2-carboxylate (1.0 g, 50%), LCMS: 386.2 [M+1]⁺

### Step-2: Synthesis of tert-butyl 6-(3-fluoro-4-(4,4,5,5-tetramethyl-1,3,2-dioxaborolan-2-yl)phenoxy)-2-azaspiro[3.3]heptane-2-carboxylate

To a stirred solution of tert-butyl 6-(4-bromo-3-fluorophenoxy)-2-azaspiro[3.3]heptane-2-carboxylate (1.0 g, 2.59 mmol, 1.0 equiv) and Bis(pinacolato)diboron (789 mg, 3.10 mmol, 1.2 equiv) in 1,4-Dioxane (12 mL) was added KOAc (635 mg, 6.47 mmol, 2.5 equiv). The reaction was purged with N₂ for 10 min, then was added Pd(dppf)Cl₂ (106 mg, 0.13 mmol, 0.05 equiv). The resulting reaction mixture was again purged with N₂ for 10 min. The reaction mixture was heated at 100° C. for 16 h. The progress of the reaction was monitored by LCMS. After completion of the reaction, reaction mixture was diluted with water (150 mL) and was extracted with EtOAc (2×250 mL). Combined organic layer was dried over Na₂SO₄, filtered and concentrated under reduced pressure. The crude product was purified by flash chromatography (0 to 20% EtOAc in hexane as an eluent) to afford tert-butyl 6-(3-fluoro-4-(4,4,5,5-tetramethyl-1,3,2-dioxaborolan-2-yl)phenoxy)-2-azaspiro[3.3]heptane-2-carboxylate (1.0 g, 89%), LCMS: 296.4 [M+1]⁺ (Boronic-Acid Fragment)

### Step-3: Synthesis of tert-butyl 6-(4-(4-chloroquinolin-6-yl)-3-fluorophenoxy)-2-azaspiro[3.3]heptane-2-carboxylate

To a stirred solution of tert-butyl 6-(3-fluoro-4-(4,4,5,5-tetramethyl-1,3,2-dioxaborolan-2-yl)phenoxy)-2-azaspiro[3.3]heptane-2-carboxylate (1.0 g, 0.2.30 mmol, 1.0 equiv) and 6-bromo-4-chloroquinoline (672 mg, 2.76 mmol, 1.2 equiv) in 1,4-dioxane:water (8.5 mL:1.5 mL) was added K₂CO₃ (793.5 mg, 5.75 mmol, 2.5 equiv). Reaction mixture was purged with N2 gas for 10 minutes and was added Pd(dppf)Cl2·DCM (94 mg, 0.11 mmol, 0.05 equiv). Reaction mixture was heated at 100° C. for 3 h. The progress of the reaction was monitored by LCMS and TLC. After completion of reaction, reaction mixture was diluted with water (75 mL) and was extracted with EtOAc (2×100 mL), combined organic layers were dried over Na₂SO₄, filtered and concentrated under reduced pressure. The crude product was purified by flash chromatography (0-2% MeOH:DCM as an eluent) to afford tert-butyl 6-(4-(4-chloroquinolin-6-yl)-3-fluorophenoxy)-2-azaspiro[3.3]heptane-2-carboxylate (700 mg, 64%), LCMS: 469.2[M+1]⁺.

### Step-4: Synthesis of tert-butyl 6-(4-(4-(benzo[d]thiazol-5-ylamino)quinolin-6-yl)-3-fluorophenoxy)-2-azaspiro[3.3]heptane-2-carboxylate

To a solution of tert-butyl 6-(4-(4-chloroquinolin-6-yl)-3-fluorophenoxy)-2-azaspiro[3.3]heptane-2-carboxylate (700 mg, 1.49 mmol, 1.0 equiv) and benzo[d]thiazol-5-amine (269 mg, 1.79 mmol, 1.2 equiv) in EtOH (3 mL) was added TFA (0.5 mL) and the reaction mixture was stirred at 80° C. for 2 h. The progress of reaction was monitored by TLC and LCMS. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 6% MeOH in DCM as an eluent) to tert-butyl 6-(4-(4-(benzo[d]thiazol-5-ylamino)quinolin-6-yl)-3-fluorophenoxy)-2-azaspiro[3.3]heptane-2-carboxylate (300 mg, 34%), LCMS: 583.2 [M+1]⁺.

### Step-5: Synthesis of N-(6-(4-(2-azaspiro[3.3]heptan-6-yloxy)-2-fluorophenyl)quinolin-4-yl)benzo[d]thiazol-5-amine

To a solution of tert-butyl 6-(4-(4-(benzo[d]thiazol-5-ylamino)quinolin-6-yl)-3-fluorophenoxy)-2-azaspiro[3.3]heptane-2-carboxylate (150 mg, 0.25 mmol, 1.0 equiv) in DCM (10 mL), was added TFA (1 mL) at 0° C. The reaction mixture was stirred at RT for 2 h. After completion of reaction, reaction mixture was concentrated under reduced pressure to obtain crude which was basified with saturated sodium bicarbonate solution (10 mL), extracted with 10% MEOH in DCM (2×20 mL), dried over anhydrous sodium sulphate, filtered and concentrated under reduced pressure. The resulting solid was washed with diethyl ether and dried under vacuum to afford (4-(4-(benzo[d]thiazol-4-ylamino)quinolin-7-yl)-3-chlorophenyl)(2,6-diazaspiro[3.3]heptan-2-yl)methanone. (30 mg, 24%), LCMS: 483.3[M+1]^{+ 1}H NMR (400 MHz, DMSO-d₆) δ 9.43 (s, 1H), 8.59-8.41 (m, 2H), 8.26 (s, 1H), 8.20 (d, J=8.3 Hz, 1H), 8.03 (d, J=1.9 Hz, 1H), 7.95 (d, J=8.3 Hz, 1H), 7.84 (d, J=8.3 Hz, 1H), 7.64 (t, J=8.9 Hz, 1H), 7.55 (dd, J=1.9, 8.9 Hz, 1H), 7.04 (d, J=5.1 Hz, 1H), 6.97-6.81 (m, 2H), 4.71 (t, J=6.7 Hz, 1H), 3.99 (br. s., 2H), 3.91 (br. s., 2H), 2.83 (br. s., 2H), 2.35-2.19 (m, 2H).

### Example 112: Synthesis of (4-(4-(benzo[d]thiazol-5-ylamino)quinolin-7-yl)-3-chlorophenyl)(2,6-diazaspiro[3.3]heptan-2-yl)methanone (Compound 312)

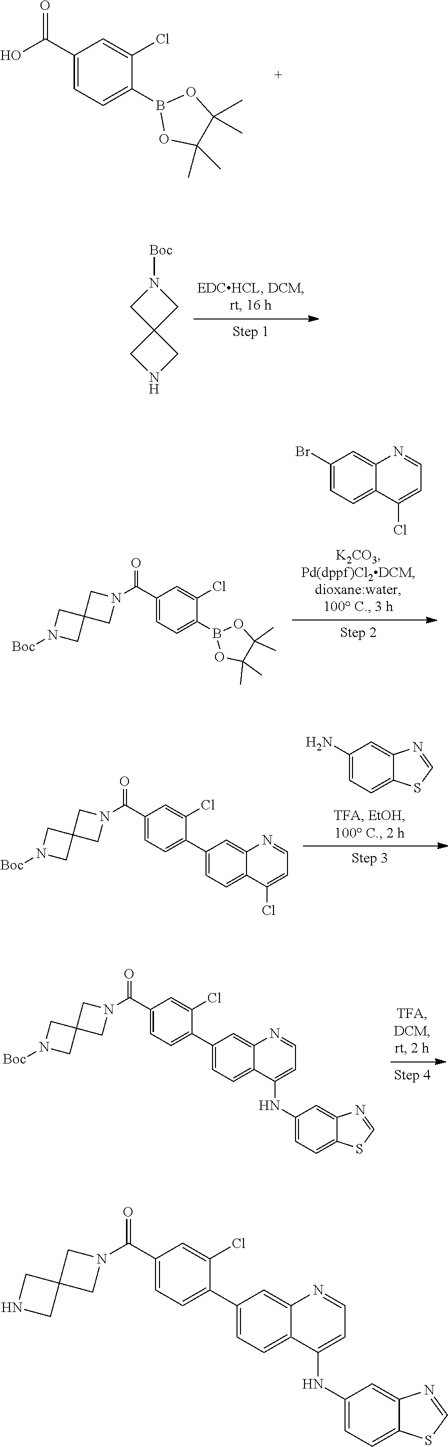

### Step-1: Synthesis of tert-butyl 6-(3-chloro-4-(4,4,5,5-tetramethyl-1,3,2-dioxaborolan-2-yl)benzoyl)-2,6-diazaspiro[3.3]heptane-2-carboxylate

To a solution 3-chloro-4-(4,4,5,5-tetramethyl-1,3,2-dioxaborolan-2-yl)benzoic acid (500 mg, 1.76 mmol, 1.0 equiv) in DCM (10 mL) was added EDC·HCl (437 mg, 2.28 mmol, 1.3 equiv) and tert-butyl 2,6-diazaspiro[3.3]heptane-2-carboxylate (420 mg, 2.11 mmol, 1.2equiv). Reaction mixture was stirred at RT for 16 h. The progress of reaction was monitored by TLC. After completion of reaction, reaction mixture was diluted with water (40 mL) and extracted with ethyl acetate (2×80 mL), washed with brine solution, dried over anhydrous sodium sulphate, filtered and concentrated under reduced pressure. The crude product was purified by flash chromatography (0 to 20% EtOAc in hexane as an eluent) to afford tert-butyl 6-(3-chloro-4-(4,4,5,5-tetramethyl-1,3,2-dioxaborolan-2-yl)benzoyl)-2,6-diazaspiro[3.3]heptane-2-carboxylate (450 mg, 55%), LCMS: 463.3[M+1]⁺

### Step-2: Synthesis of tert-butyl 6-(3-chloro-4-(4-chloroquinolin-7-yl)benzoyl)-2,6-diazaspiro[3.3]heptane-2-carboxylate

To a solution of tert-butyl 6-(3-chloro-4-(4,4,5,5-tetramethyl-1,3,2-dioxaborolan-2-yl)benzoyl)-2,6-diazaspiro[3.3]heptane-2-carboxylate (450 mg, 0.97 mmol, 1.0 equiv) in 1,4-dioxane:water (8.5 ml:1.5 ml) was added K₂CO₃ (335 mg, 2.42 mmol, 2.5 equiv) and 7-bromo-4-chloroquinoline (282 mg, 1.16 mmol, 1.2 equiv). Reaction mixture was purged with nitrogen for 5 min, then was added PdCl₂dppf·DCM (91.4 mg, 0.48 mmol, 0.05 equiv). The reaction was heated at 100° C. for 3 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 20% EtOAc in hexane as an eluent) to tert-butyl 6-(3-chloro-4-(4-chloroquinolin-7-yl)benzoyl)-2,6-diazaspiro[3.3]heptane-2-carboxylate (250 mg, 51%), LCMS: 498.1.1 M+1]⁺.

### Step-3: Synthesis of tert-butyl 6-(4-(4-(benzo[d]thiazol-5-ylamino)quinolin-7-yl)-3-chlorobenzoyl)-2,6-diazaspiro[3.3]heptane-2-carboxylate

To a solution of tert-butyl 6-(3-chloro-4-(4-chloroquinolin-7-yl)benzoyl)-2,6-diazaspiro[3.3]heptane-2-carboxylate (250 mg, 0.501 mmol, 1.0 equiv) in ethanol (10 mL) was added benzo[d]thiazol-5-amine (90 mg, 0.601 mmol, 1.0 equiv) and TFA (0.2 mL). The reaction mixture was heated at 100° C. for 2 h. After completion of reaction, reaction mixture was concentrated under reduced pressure. The resulting residue was basified with saturated sodium bicarbonate solution (20 mL), was extracted with ethyl acetate (2×80 mL), dried over anhydrous sodium sulphate, filtered and concentrated under reduced pressure. The crude product was purified by flash column chromatography (0 to 6% MeOH in DCM as an eluent) to afford tert-butyl 6-(4-(4-(benzo[d]thiazol-5-ylamino)quinolin-7-yl)-3-chlorobenzoyl)-2,6-diazaspiro[3.3]heptane-2-carboxylate (100 mg, 32%), LCMS: 612.0[M+1]⁺.

### Step-4: Synthesis of (4-(4-(benzo[d]thiazol-5-ylamino)quinolin-7-yl)-3-chlorophenyl)(2,6-diazaspiro[3.3]heptan-2-yl)methanone

To a solution tert-butyl 6-(4-(4-(benzo[d]thiazol-5-ylamino)quinolin-7-yl)-3-chlorobenzoyl)-2,6-diazaspiro[3.3]heptane-2-carboxylate (100 mg, 0.34 mmol, 1.0 equiv)) in dichloromethane (10 mL) was added TFA (1.5 mL) at 0° C. The reaction mixture was stirred at RT for 2 h. After completion of reaction, reaction mixture was concentrated under reduced pressure to obtain crude which was basified with saturated sodium bicarbonate solution (10 mL), extracted with 10% MEOH in DCM (2×20 mL), dried over anhydrous sodium sulphate, filtered and concentrated under reduced pressure. The resulting solid was washed with diethyl ether and dried under vacuum to afford (4-(4-(benzo[d]thiazol-5-ylamino)quinolin-7-yl)-3-chlorophenyl)(2,6-diazaspiro[3.3]heptan-2-yl)methanone (30 mg, 356%), LCMS: 512.1[M+1]^{+ 1}H NMR (400 MHz, DMSO-d₆) δ 9.44 (s, 1H), 9.27 (s, 1H), 8.58-8.46 (m, 2H), 8.21 (d, J=8.3 Hz, 1H), 7.94 (s, 1H), 7.83 (s, 1H), 7.73 (s, 1H), 7.69-7.62 (m, 2H), 7.55 (s, 1H), 7.58 (s, 1H), 7.06 (d, J=5.1 Hz, 1H), 4.54 (br. s., 1H), 4.49 (br. s., 1H), 4.23 (br. s., 1H), 4.17 (s, 1H), 3.67 (br. s., 4H).

### Example 113: Synthesis of (4-(4-(benzo[d]thiazol-5-ylamino)quinolin-6-yl)-3-fluorophenyl)((1R,5S)-3,6-diazabicyclo[3.1.1]heptan-6-yl)methanone (Compound 313)

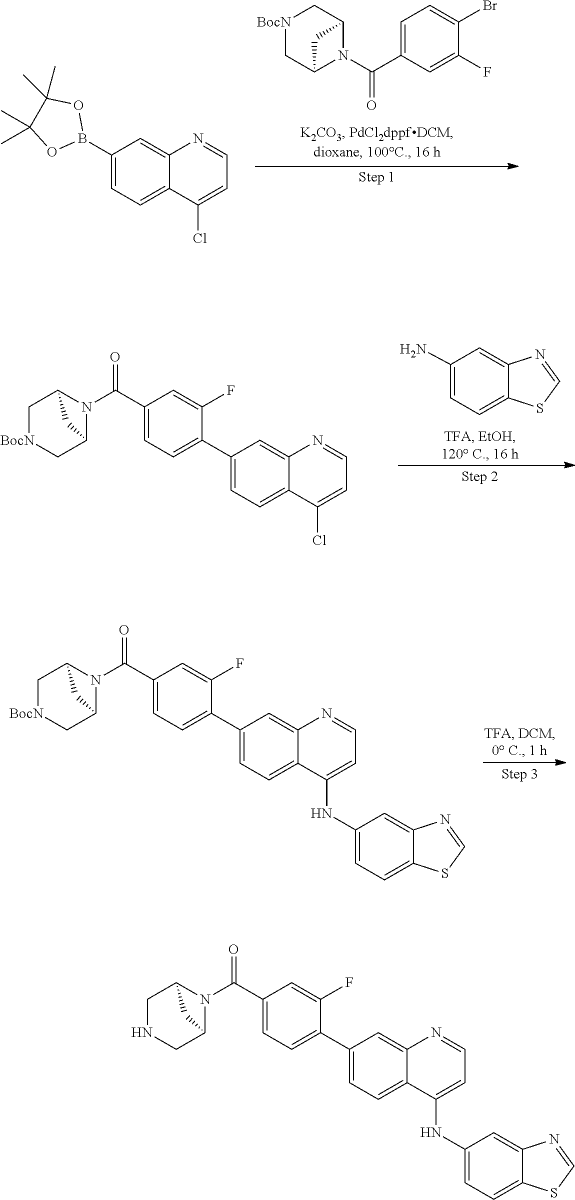 [SMILES: CC1(C)OB(c2ccc3c(Cl)ccnc3c2)OC1(C)C ; CC(C)(C)OC(=O)N1C[C@H]2C[C@@H](C1)N2C(=O)c1ccc(Br)c(F)c1 ; CC(C)(C)OC(=O)N1C[C@H]2C[C@@H](C1)N2C(=O)c1ccc(-c2ccc3c(Cl)ccnc3c2)c(F)c1 ; Nc1ccc2scnc2c1 ; CC(C)(C)OC(=O)N1C[C@H]2C[C@@H](C1)N2C(=O)c1ccc(-c2ccc3c(Nc4ccc5scnc5c4)ccnc3c2)c(F)c1 ; O=C(c1ccc(-c2ccc3c(Nc4ccc5scnc5c4)ccnc3c2)c(F)c1)N1[C@@H]2CNC[C@H]1C2]

### Step-1: Synthesis of tert-butyl (1R,5S)-6-(4-(4-chloroquinolin-7-yl)-3-fluorobenzoyl)-3,6-diazabicyclo[3.1.1]heptane-3-carboxylate

To a solution of tert-butyl (1R,5S)-6-(4-bromo-3-fluorobenzoyl)-3,6-diazabicyclo[3.1.1]heptane-3-carboxylate (250 mg, 0.63 mmol, 1.0 equiv) in 1,4-dioxane:water (6 mL:2 mL) was added K₂CO₃ (173 mg, 1.25 mmol, 2.0 equiv) and 4-chloro-7-(4,4,5,5-tetramethyl-1,3,2-dioxaborolan-2-yl)quinoline (181 mg, 0.63 mmol, 1.0 equiv). Reaction mixture was purged with nitrogen for 5 min, then was added PdCl₂dppf·DCM (25 mg, 0.03 mmol, 0.05 equiv). The reaction was heated at 100° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure. The crude product was purified by flash chromatography (0-2% MeOH:DCM as an eluent) to afford tert-butyl (1R,5S)-6-(4-(4-chloroquinolin-7-yl)-3-fluorobenzoyl)-3,6-diazabicyclo[3.1.1]heptane-3-carboxylate (100 mg, 33%), LCMS: 482.1[M+1]⁺.

### Step-2: Synthesis of tert-butyl (1R,5S)-6-(4-(4-(benzo[d]thiazol-5-ylamino)quinolin-7-yl)-3-fluorobenzoyl)-3,6-diazabicyclo[3.1.1]heptane-3-carboxylate

To a solution of tert-butyl (1R,5S)-6-(4-(4-chloroquinolin-7-yl)-3-fluorobenzoyl)-3,6-diazabicyclo[3.1.1]heptane-3-carboxylate (100 mg, 0.20 mmol, 1.0 equiv) in ethanol (5 mL) was added benzo[d]thiazol-5-amine (46 mg, 0.31 mmol, 1.5 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×50 mL), dried over anhydrous sodium sulphate, filtered and concentrated under reduced pressure. The crude product was purified by flash column chromatography (0 to 5% MeOH in DCM as an eluent) to afford tert-butyl (1R,5S)-6-(4-(4-(benzo[d]thiazol-5-ylamino)quinolin-7-yl)-3-fluorobenzoyl)-3,6-diazabicyclo[3.1.1]heptane-3-carboxylate (80 mg, 65%), LCMS: 596.4[M+1]⁺.

### Step-3: Synthesis of (4-(4-(benzo[d]thiazol-5-ylamino)quinolin-7-yl)-3-fluorophenyl)((1R,5S)-3,6-diazabicyclo[3.1.1]heptan-6-yl)methanone

To a solution of tert-butyl (1R,5S)-6-(4-(4-chloroquinolin-6-yl)-3-fluorobenzoyl)-3,6-diazabicyclo[3.1.1]heptane-3-carboxylate (80 mg, 0.13 mmol, 1.0 equiv) in DCM (5 mL) was added TFA (1 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with DCM (2×40 mL), dried over anhydrous sodium sulphate, filtered and concentrated under reduced pressure. The crude product was purified by flash column chromatography (0 to 8% MeOH in DCM) to afford (4-(4-(benzo[d]thiazol-5-ylamino)quinolin-7-yl)-3-fluorophenyl)((1R,5S)-3,6-diazabicyclo[3.1.1]heptan-6-yl)methanone (61 mg, 92%), LCMS: 496.3[M+1]^{+ 1}H NMR (400 MHz, DMSO-d₆) δ 9.54 (br. s., 1H) 9.17 (br. s., 1H) 9.00 (br. s., 1H) 8.58 (br. s., 1H) 8.41 (d, J=6.99 Hz, 1H) 8.24 (s, 1H) 8.26 (s, 1H) 8.14 (br. s., 1H) 7.88 (br. s., 1H) 7.73 (d, J=8.27 Hz, 2H) 7.63 (br. s., 1H) 6.97 (br. s., 1H) 4.54 (s, 2H) 3.63 (br. s., 2H) 3.39 (br. s., 1H) 2.91 (br. s., 1H) 2.33 (br. s., 1H) 1.90 (d, J=8.90 Hz, 1H).

### Example 114: Synthesis of (4-(4-(benzo[d]thiazol-5-ylamino)quinolin-7-yl)-3-fluorophenyl)(2,8-diazaspiro[4.5]decan-8-yl)methanone (Compound 314)

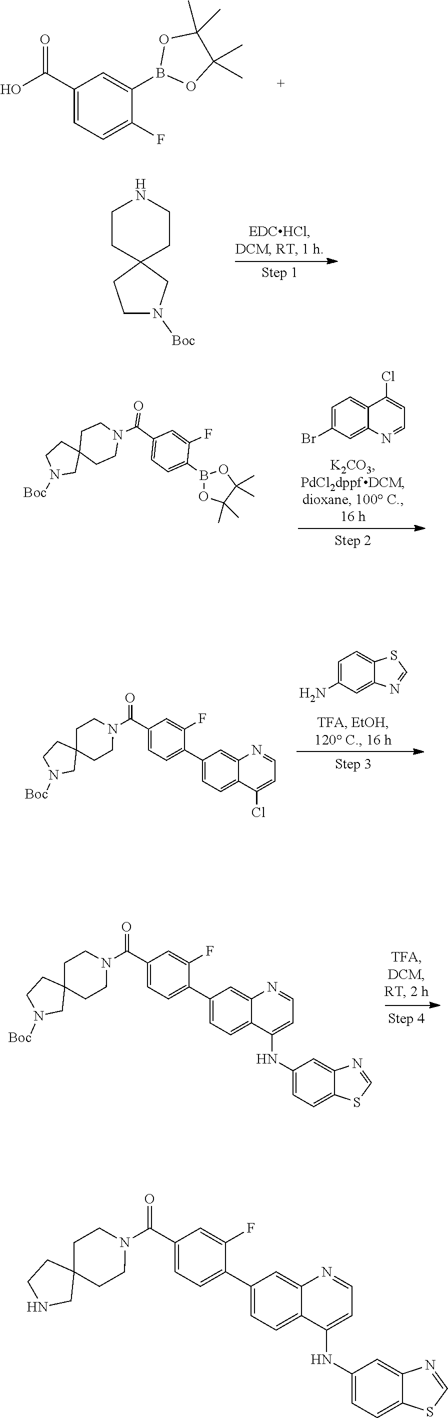

### Step-1:Synthesis of tert-butyl 8-(3-fluoro-4-(4,4,5,5-tetramethyl-1,3,2-dioxaborolan-2-yl)benzoyl)-2,8-diazaspiro[4.5]decane-2-carboxylate

To a solution of 4-fluoro-3-(4,4,5,5-tetramethyl-1,3,2-dioxaborolan-2-yl)benzoic acid (300 mg, 1.12 mmol, 1.0 equiv) in DCM (15 mL) was added EDC·HCl (258 mg, 1.3 mmol, 1.2 equiv) and tert-butyl 2,8-diazaspiro[4.5]decane-2-carboxylate (270 mg, 1.12 mmol, 1.0 equiv). Reaction mixture was stirred at RT for 1 h. The progress of reaction was monitored by TLC. After completion of reaction, reaction mixture was diluted with water (40 mL) and extracted with ethyl acetate (2×80 mL), washed with brine solution, dried over anhydrous sodium sulphate, filtered and concentrated under reduced pressure. The crude product was purified by flash chromatography (0 to 20% EtOAc in hexane as an eluent) to afford tert-butyl 8-(3-fluoro-4-(4,4,5,5-tetramethyl-1,3,2-dioxaborolan-2-yl)benzoyl)-2,8-diazaspiro[4.5]decane-2-carboxylate (200 mg, 36%), LCMS: 351.3[M+1]⁺[Boronic acid fragment]

### Step-2: Synthesis of tert-butyl 8-(4-(4-chloroquinolin-7-yl)-3-fluorobenzoyl)-2,8-diazaspiro[4.5]decane-2-carboxylate

To a solution of tert-butyl 8-(3-fluoro-4-(4,4,5,5-tetramethyl-1,3,2-dioxaborolan-2-yl)benzoyl)-2,8-diazaspiro[4.5]decane-2-carboxylate (200 mg, 0.41 mmol, 1.0 equiv) in 1,4-dioxane:water (8.5 ml:1.5 ml) was added K₂CO₃ (84 mg, 0.61 mmol, 1.5 equiv) and 7-bromo-4-chloroquinoline (99 mg, 0.41 mmol, 1.0 equiv). Reaction mixture was purged with nitrogen for 5 min, then was added PdCl₂dppf·DCM (16 mg, 0.020 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 20% EtOAc in hexane as an eluent) to afford tert-butyl 8-(4-(4-chloroquinolin-7-yl)-3-fluorobenzoyl)-2,8-diazaspiro[4.5]decane-2-carboxylate (150 mg, 70%), LCMS: 524.3 [M+1]⁺.

### Step-3: Synthesis of tert-butyl 8-(4-(4-(benzo[d]thiazol-5-ylamino)quinolin-7-yl)-3-fluorobenzoyl)-2,8-diazaspiro[4.5]decane-2-carboxylate

To a solution of tert-butyl 8-(4-(4-chloroquinolin-7-yl)-3-fluorobenzoyl)-2,8-diazaspiro[4.5]decane-2-carboxylate (150 mg, 0.28 mmol, 1.0 equiv) in ethanol (10 mL) was added benzo[d]thiazol-5-amine (43 mg, 0.28 mmol, 1.0 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×80 mL), dried over anhydrous sodium sulphate, filtered and concentrated under reduced pressure. The crude product was purified by flash column chromatography (0 to 6% MeOH in DCM as an eluent) to afford tert-butyl 8-(4-(4-(benzo[d]thiazol-5-ylamino)quinolin-7-yl)-3-fluorobenzoyl)-2,8-diazaspiro[4.5]decane-2-carboxylate (50 mg, 27%), LCMS: 638.1 [M+1]⁺.

### Step-4: Synthesis of (4-(4-(benzo[d]thiazol-5-ylamino)quinolin-7-yl)-3-fluorophenyl)(2,8-diazaspiro[4.5]decan-8-yl)methanone

To a solution of tert-butyl 8-(4-(4-(benzo[d]thiazol-5-ylamino)quinolin-7-yl)-3-fluorobenzoyl)-2,8-diazaspiro[4.5]decane-2-carboxylate (50 mg, 0.08 mmol, 1.0 equiv) in dichloromethane (10 mL) was added TFA (0.5 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with ethyl acetate (2×40 mL), dried over anhydrous sodium sulphate, filtered and concentrated under reduced pressure. The crude product was purified by flash column chromatography (0 to 10% MeOH in DCM as an eluent) to afford (4-(4-(benzo[d]thiazol-5-ylamino)quinolin-7-yl)-3-fluorophenyl)(2,8-diazaspiro[4.5]decan-8-yl)methanone (10 mg, 24%), LCMS: 538.2 [M+1]⁺. ¹H NMR (400 MHz, DMSO-d₆) δ 1.23 (s, 4H) 1.53 (s, 6H) 1.77 (br. s., 1H) 2.67 (br. s., 4H) 7.05 (d, J=5.72 Hz, 1H) 7.36 (s, 1H) 7.57 (s, 1H) 7.79 (d, J=8.27 Hz, 2H) 8.05 (s, 2H) 8.10 (s, 1H) 8.21 (d, J=8.27 Hz, 2H) 8.54 (d, J=5.09 Hz, 1H) 9.27 (s, 1H) 9.43 (s, 1H).

### Example 115: Synthesis of (4-(4-(benzo[d]thiazol-5-ylamino)quinolin-6-yl)-3-fluorophenyl)(3,3,4-trimethylpiperazin-1-yl)methanone (Compound 315)

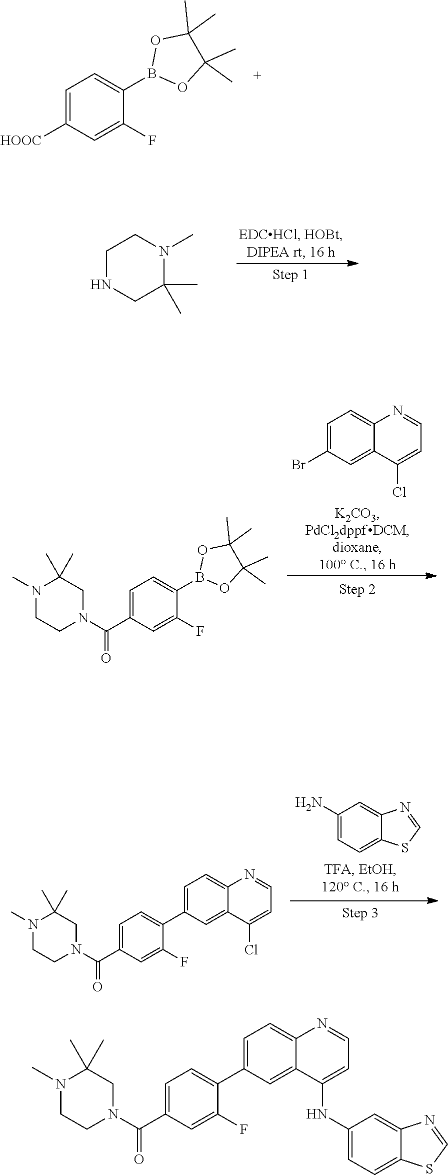

### Step-1: Synthesis of (3-fluoro-4-(4,4,5,5-tetramethyl-1,3,2-dioxaborolan-2-yl)phenyl)(3,3,4-trimethylpiperazin-1-yl)methanone

To a solution of 3-fluoro-4-(4,4,5,5-tetramethyl-1,3,2-dioxaborolan-2-yl)benzoic acid (250 mg, 0.93 mmol, 1.0 equiv) in THF (10 mL) was added EDC·HCl (234 mg, 1.22 mmol, 1.3 equiv) and HOBt (165 mg, 1.22 mmol, 1.3 equiv). Reaction was stirred at RT for 1 h. Then added, DIPEA (0.5 mL, 2.81 mmol, 3.0 equiv) and 1,2,2-trimethylpiperazine (188 mg, 0.93 mmol, 1.0 equiv). Reaction mixture was stirred at RT for 16 h. The progress of reaction was monitored by TLC. After completion of reaction, reaction mixture was diluted with water (20 mL) and extracted with ethyl acetate (2×40 mL), washed with brine solution, dried over anhydrous sodium sulphate, filtered and concentrated under reduced pressure. The crude product was purified by flash chromatography (0-70% ethyl acetate:hexane as an eluent) to afford (3-fluoro-4-(4,4,5,5-tetramethyl-1,3,2-dioxaborolan-2-yl)phenyl)(3,3,4-trimethylpiperazin-1-yl)methanone (310 mg, 87%), LCMS: 295.3[boronic acid fragment].

### Step-2: Synthesis of (4-(4-chloroquinolin-6-yl)-3-fluorophenyl)(3,3,4-trimethylpiperazin-1-yl)methanone

To a solution of 3-fluoro-4-(4,4,5,5-tetramethyl-1,3,2-dioxaborolan-2-yl)phenyl)(3,3,4-trimethylpiperazin-1-yl)methanone (310 mg, 0.82 mmol, 1.0 equiv) in 1,4-dioxane:water (6 mL:2 mL) was added K₂CO₃ (227 mg, 1.64 mmol, 2.0 equiv) and 6-bromo-4-chloroquinoline (199 mg, 0.82 mmol, 1.0 equiv). Reaction mixture was purged with nitrogen for 5 min, then was added PdCl₂dppf·DCM (33 mg, 0.04 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×60 mL), dried over anhydrous sodium sulphate, filtered and concentrated under reduced pressure. The crude product was purified by flash chromatography (0-2% MeOH:DCM as an eluent) to afford (4-(4-chloroquinolin-6-yl)-3-fluorophenyl)(3,3,4-trimethylpiperazin-1-yl)methanone (170 mg, 50%), LCMS: 412.2[M+1]⁺.

### Step-3: Synthesis of (4-(4-(benzo[d]thiazol-5-ylamino)quinolin-6-yl)-3-fluorophenyl)(3,3,4-trimethylpiperazin-1-yl)methanone

To a solution of (4-(4-chloroquinolin-6-yl)-3-fluorophenyl)(3,3,4-trimethylpiperazin-1-yl)methanone (170 mg, 0.41 mmol, 1.0 equiv) in ethanol (5 mL) was added benzo[d]thiazol-5-amine (92 mg, 0.61 mmol, 1.5 equiv) and TFA (0.1 mL). The reaction mixture was heated at 120° C. for 16 h. After completion of reaction, reaction mixture was concentrated under reduced pressure. The resulting residue was basified with saturated sodium bicarbonate solution (20 mL), was extracted with ethyl acetate (2×50 mL), dried over anhydrous sodium sulphate, filtered and concentrated under reduced pressure. The crude product was purified by flash column chromatography (0 to 7% MeOH in DCM as an eluent) to afford (4-(4-(benzo[d]thiazol-5-ylamino)quinolin-6-yl)-3-fluorophenyl)(3,3,4-trimethylpiperazin-1-yl)methanone (57 mg, 26%), LCMS: 526.2 [M+1]^{+ 1}H NMR (400 MHz, DMSO-d₆) δ 9.43 (s, 1H) 9.35 (br. s., 1H) 8.67 (s, 1H) 8.52 (d, J=5.09 Hz, 1H) 8.21 (d, J=8.90 Hz, 1H) 8.04 (s, 1H) 7.99 (d, J=8.90 Hz, 1H) 7.94 (d, J=8.90 Hz, 1H) 7.81 (t, J=7.95 Hz, 1H) 7.56 (d, J=8.27 Hz, 1H) 7.40 (br. s., 2H) 7.05 (d, J=5.09 Hz, 1H) 3.62 (br. s., 2H) 3.39 (br. s., 1H) 3.16 (br. s., 3H) 2.16 (br. s., 3H) 1.03 (br. s., 3H) 0.87 (br. s., 3H).

### Example 116: Synthesis of (4-(4-(benzo[d]thiazol-5-ylamino)quinolin-6-yl)-3-fluorophenyl)(6-(methylsulfonyl)-2,6-diazaspiro[3.3]heptan-2-yl)methanone (Compound 316)

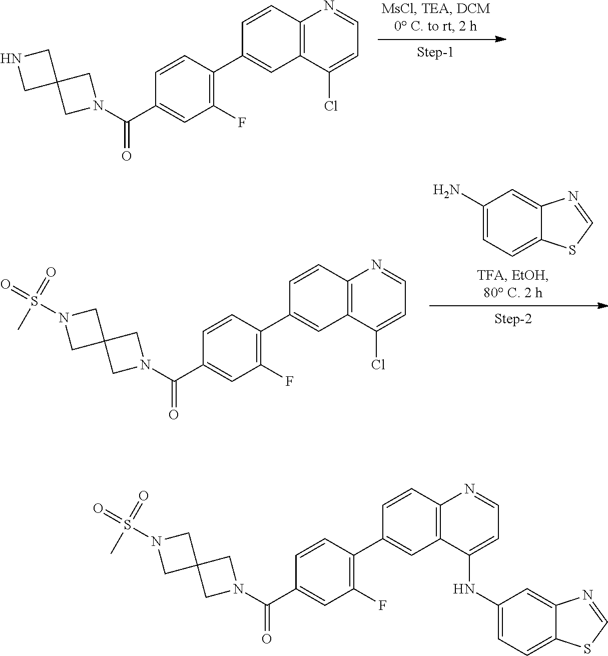 [SMILES: O=C(c1ccc(-c2ccc3nccc(Cl)c3c2)c(F)c1)N1CC2(CNC2)C1 ; Nc1ccc2scnc2c1 ; CS(=O)(=O)N1CC2(CN(C(=O)c3ccc(-c4ccc5nccc(Cl)c5c4)c(F)c3)C2)C1 ; CS(=O)(=O)N1CC2(CN(C(=O)c3ccc(-c4ccc5nccc(Nc6ccc7scnc7c6)c5c4)c(F)c3)C2)C1]

### Step-1: Synthesis of (4-(4-chloroquinolin-6-yl)-3-fluorophenyl)(6-(methylsulfonyl)-2,6-diazaspiro[3.3]heptan-2-yl)methanone

To a solution of (4-(4-chloroquinolin-6-yl)-3-fluorophenyl)(2,6-diazaspiro[3.3]heptan-2-yl)methanone (350 mg, 0.91 mmol, 1.0 equiv) in DCM (5 mL) was added TEA (0.2 mL) followed by the addition of mesyl chloride (1 mL) at 0° C. The reaction mixture was further stirred at 0° C. for 10 min and then was allowed to stir at RT for 2 h. product formation was confirmed by TLC and LCMS. After completion of reaction, reaction mixture was basified by saturated sodium bicarbonate solution (10 mL), extracted with DCM (2×50 mL), dried over anhydrous sodium sulphate, filtered and concentrated under reduced pressure. The crude product was purified by flash chromatography (0 to 30% ethyl acetate in hexane as an eluent) to afford (4-(4-chloroquinolin-6-yl)-3-fluorophenyl)(6-(methylsulfonyl)-2,6-diazaspiro[3.3]heptan-2-yl)methanone (156 mg, 36%), LCMS: 460.1[M+1]⁺

### Step-2: Synthesis of (4-(4-(benzo[d]thiazol-5-ylamino)quinolin-6-yl)-3-fluorophenyl)(6-(methylsulfonyl)-2,6-diazaspiro[3.3]heptan-2-yl)methanone

To a solution of (4-(4-chloroquinolin-6-yl)-3-fluorophenyl)(6-(methylsulfonyl)-2,6-diazaspiro[3.3]heptan-2-yl)methanone (150 mg, 0.33 mmol, 1.0 equiv) in ethanol (5 mL) was added benzo[d]thiazol-5-amine (49 mg, 0.99 mmol, 1.5 equiv) and TFA (0.2 mL). The reaction mixture was heated at 80° C. for 2 h. After completion of reaction, reaction mixture was concentrated under reduced pressure. The resulting residue was basified with saturated sodium bicarbonate solution (20 mL), was extracted with DCM (2×40 mL), dried over anhydrous sodium sulphate, filtered and concentrated under reduced pressure. The crude product was purified by reversed phase HPLC to afford (4-(4-(benzo[d]thiazol-5-ylamino)quinolin-6-yl)-3-fluorophenyl)(6-(methylsulfonyl)-2,6-diazaspiro[3.3]heptan-2-yl)methanone (19 mg, 10%), LCMS: 574.1 [M+1]^{+ 1}H NMR (400 MHz, DMSO-d₆) δ 9.43 (s, 1H), 9.31 (br. s., 1H), 8.67 (s, 1H), 8.52 (d, J=5.09 Hz, 1H), 8.13-8.26 (m, 2H), 7.88-8.11 (m, 4H), 7.78-7.88 (m, 1H), 7.47-7.69 (m, 4H), 7.06 (d, J=5.09 Hz, 1H), 4.57 (s, 2H), 4.25 (s, 3H), 4.07 (d, J=8.27 Hz, 5H), 2.99 (s, 3H).

### Example 117: Synthesis of (4-(4-(benzo[d]thiazol-5-ylamino)quinolin-6-yl)-3-fluorophenyl)(4-hydroxypiperidin-1-yl)methanone (Compound 317)

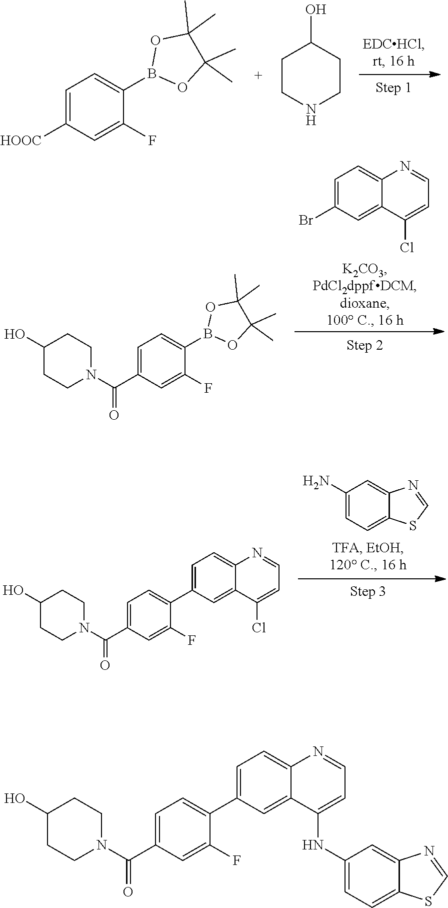

### Step-1: Synthesis of (3-fluoro-4-(4,4,5,5-tetramethyl-1,3,2-dioxaborolan-2-yl)phenyl)(4-hydroxypiperidin-1-yl)methanone

To a solution of 3-fluoro-4-(4,4,5,5-tetramethyl-1,3,2-dioxaborolan-2-yl)benzoic acid (200 mg, 0.75 mmol, 1.0 equiv) in DCM (10 mL) was added EDC·HCl (187 mg, 0.97 mmol, 1.3 equiv) Reaction was stirred at RT for 1 h. Then was added piperidin-4-ol (76 mg, 0.75 mmol, 1.0 equiv). Reaction was stirred at RT for 16 h. The progress of reaction was monitored by TLC. After completion of reaction, reaction mixture was diluted with water (20 mL) and extracted with ethyl acetate (2×40 mL), washed with brine solution, dried over anhydrous sodium sulphate, filtered and concentrated under reduced pressure. The crude product was purified by flash chromatography (0-70% ethyl acetate:hexane as an eluent) to afford (3-fluoro-4-(4,4,5,5-tetramethyl-1,3,2-dioxaborolan-2-yl)phenyl)(4-hydroxypiperidin-1-yl)methanone (260 mg, 99%), LCMS: 268.3 [boronic acid fragment]

### Step-2: Synthesis of (4-(4-chloroquinolin-6-yl)-3-fluorophenyl)(4-hydroxypiperidin-1-yl)methanone

To a solution of (3-fluoro-4-(4,4,5,5-tetramethyl-1,3,2-dioxaborolan-2-yl)phenyl)(4-hydroxypiperidin-1-yl)methanone (260 mg, 0.74 mmol, 1.0 equiv) in 1,4-dioxane:water (6 mL:2 mL) was added K₂CO₃ (205 mg, 1.49 mmol, 2.0 equiv) and 6-bromo-4-chloroquinoline (180 mg, 0.74 mmol, 1.0 equiv). Reaction mixture was purged with nitrogen for 5 min, then was added PdCl₂dppf·DCM (30 mg, 0.03 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×60 mL), dried over anhydrous sodium sulphate, filtered and concentrated under reduced pressure. The crude product was purified by flash chromatography (0-2% MeOH:DCM as an eluent) to afford (4-(4-chloroquinolin-6-yl)-3-fluorophenyl)(4-hydroxypiperidin-1-yl)methanone (120 mg, 42%), LCMS: 385.3[M+1]⁺.

### Step-3: Synthesis of (4-(4-(benzo[d]thiazol-5-ylamino)quinolin-6-yl)-3-fluorophenyl)(4-hydroxypiperidin-1-yl)methanone

To a solution of (4-(4-chloroquinolin-6-yl)-3-fluorophenyl)(4-hydroxypiperidin-1-yl)methanone (120 mg, 0.31 mmol, 1.0 equiv) in ethanol (5 mL) was added benzo[d]thiazol-5-amine (70 mg, 0.47 mmol, 1.5 equiv) and TFA (0.1 mL). The reaction mixture was heated at 120° C. for 2 h. After completion of reaction, reaction mixture was concentrated under reduced pressure. The resulting residue was basified with saturated sodium bicarbonate solution (20 mL), was extracted with ethyl acetate (2×50 mL), dried over anhydrous sodium sulphate, filtered and concentrated under reduced pressure. The crude product was purified by flash column chromatography (0 to 7% MeOH in DCM as an eluent) to afford (4-(4-(benzo[d]thiazol-5-ylamino)quinolin-6-yl)-3-fluorophenyl)(4-hydroxypiperidin-1-yl)methanone (58 mg, 37%), LCMS: 499.2[M+1]^{+ 1}H NMR (400 MHz, DMSO-d₆) δ 9.40 (s, 1H) 8.64 (s, 1H) 8.50 (d, J=5.72 Hz, 2H) 8.19 (d, J=8.90 Hz, 1H) 8.03 (s, 1H) 7.91-7.99 (m, 2H) 7.78 (t, J=7.95 Hz, 1H) 7.55 (d, J=8.27 Hz, 1H) 7.33-7.43 (m, 2H) 7.05 (d, J=5.09 Hz, 1H) 4.81 (s., 1H) 4.01 (br. s., 1H) 3.75 (br. s., 1H) 3.34 (br. s., 1H) 1.81 (br. s., 2H) 1.73 (br. s., 2H) 1.37 (br. s., 1H).

### Example 118: (4-(4-(benzo[d]thiazol-5-ylamino)quinolin-6-yl)-3-fluorophenyl)(4-hydroxy-4-methylpiperidin-1-yl)methanone (Compound 318)

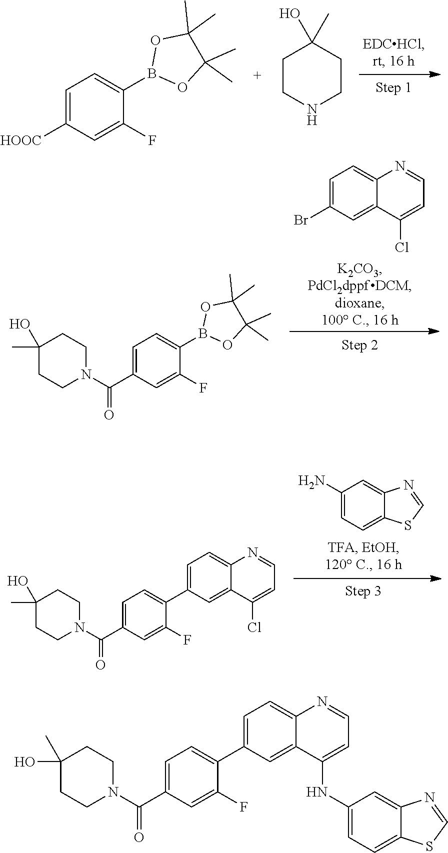

### Step-1: Synthesis of (3-fluoro-4-(4,4,5,5-tetramethyl-1,3,2-dioxaborolan-2-yl)phenyl)(4-hydroxy-4-methylpiperidin-1-yl)methanone

To a solution of 3-fluoro-4-(4,4,5,5-tetramethyl-1,3,2-dioxaborolan-2-yl)benzoic acid (200 mg, 0.75 mmol, 1.0 equiv) in DCM (10 mL) was added EDC·HCl (187 mg, 0.97 mmol, 1.3 equiv) Reaction was stirred at RT for 1 h. Then was added 4-methylpiperidin-4-ol (86 mg, 0.75 mmol, 1.0 equiv). Reaction was stirred at RT for 16 h. The progress of reaction was monitored by TLC. After completion of reaction, reaction mixture was diluted with water (20 mL) and extracted with ethyl acetate (2×40 mL), washed with brine solution, dried over anhydrous sodium sulphate, filtered and concentrated under reduced pressure. The crude product was purified by flash chromatography (0-70% ethyl acetate:hexane as an eluent) to afford (3-fluoro-4-(4,4,5,5-tetramethyl-1,3,2-dioxaborolan-2-yl)phenyl)(4-hydroxy-4-methylpiperidin-1-yl)methanone (270 mg, 99%), LCMS: 282.4[boronic acid fragment].

### Step-2: Synthesis of (4-(4-chloroquinolin-6-yl)-3-fluorophenyl)(4-hydroxy-4-methylpiperidin-1-yl)methanone

To a solution of (3-fluoro-4-(4,4,5,5-tetramethyl-1,3,2-dioxaborolan-2-yl)phenyl)(4-hydroxy-4-methylpiperidin-1-yl)methanone (270 mg, 0.74 mmol, 1.0 equiv) in 1,4-dioxane:water (6 mL:2 mL) was added K₂CO₃ (205 mg, 1.49 mmol, 2.0 equiv) and 6-bromo-4-chloroquinoline (180 mg, 0.74 mmol, 1.0 equiv). Reaction mixture was purged with nitrogen for 5 min, then was added PdCl₂dppf·DCM (30 mg, 0.03 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×60 mL), dried over anhydrous sodium sulphate, filtered and concentrated under reduced pressure. The crude product was purified by flash chromatography (0-2% MeOH:DCM as an eluent) to afford (4-(4-chloroquinolin-6-yl)-3-fluorophenyl)(4-hydroxy-4-methylpiperidin-1-yl)methanone (135 mg, 45%), LCMS: 399.3[M+1]⁺.

### Step-3: Synthesis of (4-(4-(benzo[d]thiazol-5-ylamino)quinolin-6-yl)-3-fluorophenyl)(4-hydroxy-4-methylpiperidin-1-yl)methanone

To a solution of (4-(4-chloroquinolin-6-yl)-3-fluorophenyl)(4-hydroxy-4-methylpiperidin-1-yl)methanone (135 mg, 0.34 mmol, 1.0 equiv) in ethanol (5 mL) was added benzo[d]thiazol-5-amine (76 mg, 0.50 mmol, 1.5 equiv) and TFA (0.1 mL). The reaction mixture was heated at 120° C. for 2 h. After completion of reaction, reaction mixture was concentrated under reduced pressure. The resulting residue was basified with saturated sodium bicarbonate solution (20 mL), was extracted with ethyl acetate (2×50 mL), dried over anhydrous sodium sulphate, filtered and concentrated under reduced pressure. The crude product was purified by flash column chromatography (0 to 7% MeOH in DCM as an eluent) to afford (4-(4-(benzo[d]thiazol-5-ylamino)quinolin-6-yl)-3-fluorophenyl)(4-hydroxy-4-methylpiperidin-1-yl)methanone (15 mg, 8%), LCMS: 513.3[M+1]^{+ 1}H NMR (400 MHz, DMSO-d₆) δ 9.43 (s, 1H) 9.30 (s, 1H) 8.65 (s, 1H) 8.52 (d, J=5.09 Hz, 1H) 8.21 (d, J=8.27 Hz, 1H) 8.04 (s, 1H) 7.99 (d, J=8.90 Hz, 1H) 7.92 (d, J=8.90 Hz, 1H) 7.79 (t, J=7.95 Hz, 1H) 7.56 (d, J=8.27 Hz, 1H) 7.35-7.45 (m, 2H) 7.06 (d, J=5.09 Hz, 1H) 4.45 (s, 1H) 4.10 (br. s., 1H) 3.40 (s, 2H) 3.21 (s, 2H) 1.54 (s, 2H) 1.51 (s, 1H) 1.17 (s, 3H).

### Example 119: Synthesis of 1-(4-(4-(benzo[d]thiazol-5-ylamino)quinolin-6-yl)-3-fluorobenzyl)-4-methylpiperidin-4-ol (Compound 319)

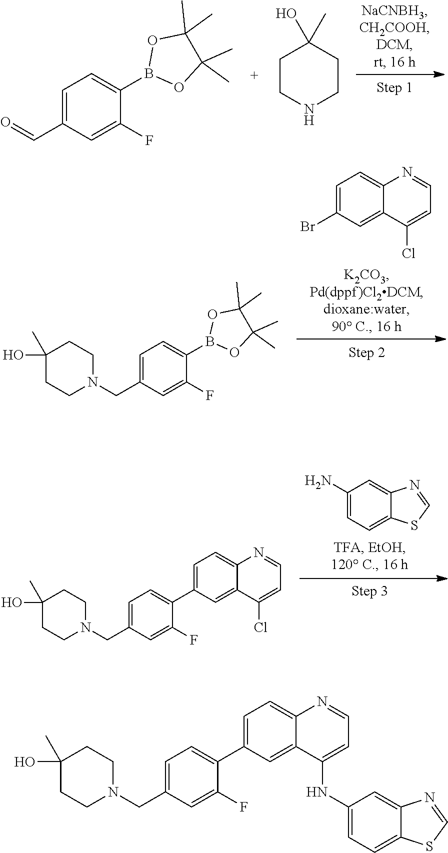

### Step-1: Synthesis of 1-(3-fluoro-4-(4,4,5,5-tetramethyl-1,3,2-dioxaborolan-2-yl)benzyl)-4-methylpiperidin-4-ol

To a solution of 3-fluoro-4-(4,4,5,5-tetramethyl-1,3,2-dioxaborolan-2-yl)benzaldehyde (200 mg, 0.8 mmol, 1.0 equiv) in DCM (5 mL) was added CH₃COOH (0.1 mL) and 4-methylpiperidin-4-ol (184 mg, 1.60 mmol, 2.0 equiv). Reaction mixture was stirred at RT for 1 h. NaCNBH3 (75 mg, 1.2 mmol, 1.5 equiv) was added at 0° C. Reaction mixture was stirred at RT for overnight. The progress of reaction was monitored by TLC. After completion of reaction, reaction mixture was diluted with water (30 mL) and extracted with DCM (2×60 ml), washed with brine solution, dried over anhydrous sodium sulphate, filtered and concentrated under reduced pressure to afford 1-(3-fluoro-4-(4,4,5,5-tetramethyl-1,3,2-dioxaborolan-2-yl)benzyl)-4-methylpiperidin-4-ol (240 mg, 86%), LCMS: 268.4 [M+1]⁺[Boronic acid fragment].

### Step-2: Synthesis of 1-(4-(4-chloroquinolin-6-yl)-3-fluorobenzyl)-4-methylpiperidin-4-ol

To a solution of 1-(3-fluoro-4-(4,4,5,5-tetramethyl-1,3,2-dioxaborolan-2-yl)benzyl)-4-methylpiperidin-4-ol (240 mg, 0.69 mmol, 1.0 equiv) in 1,4-dioxane:water (8 ml:1 ml) was added K₂CO₃ (189 mg, 1.37 mmol, 2.0 equiv) and 6-bromo-4-chloroquinoline (166 mg, 0.69 mmol, 1.0 equiv). Reaction mixture was purged with nitrogen for 5 min, then was added PdCl₂dppf·DCM (28 mg, 0.03 mmol, 0.05 equiv). The reaction was heated at 90° C. for 16 h. The progress of reaction was monitored by TLC. After completion of reaction, reaction mixture was diluted with water (40 mL) and extracted with ethyl acetate (2×60 mL), dried over anhydrous sodium sulphate, filtered and concentrated under reduced pressure. The crude product was purified by flash chromatography (0-5% MeOH:DCM as an eluent) to afford 1-(4-(4-chloroquinolin-6-yl)-3-fluorobenzyl)-4-methylpiperidin-4-ol (75 mg, 28%), LCMS: 385.3 M+1]⁺.

### Step-3: Synthesis of 1-(4-(4-(benzo[d]thiazol-5-ylamino)quinolin-6-yl)-3-fluorobenzyl)-4-methylpiperidin-4-ol

To a solution of 1-(4-(4-chloroquinolin-6-yl)-3-fluorobenzyl)-4-methylpiperidin-4-ol (75 mg, 0.19 mmol, 1.0 equiv) in ethanol (10 mL) was added benzo[d]thiazol-5-amine (29 mg, 0.19 mmol, 1.0 equiv) and TFA (0.05 mL). The reaction mixture was heated at 120° C. for 2 h. After completion of reaction, reaction mixture was concentrated under reduced pressure. The resulting residue was basified with saturated sodium bicarbonate solution (20 mL), was extracted with ethyl acetate (2×50 mL), dried over anhydrous sodium sulphate, filtered and concentrated under reduced pressure. The crude product was purified by reversed phase HPLC to afford 1-(4-(4-(benzo[d]thiazol-5-ylamino)quinolin-6-yl)-3-fluorobenzyl)-4-methylpiperidin-4-ol (8 mg, 8%), LCMS: 499.2[M+1]⁺. ¹H NMR (400 MHz, DMSO-d₆) δ 9.43 (s, 1H) 9.29 (br. s., 1H) 8.60 (s, 1H) 8.50 (d, J=5.09 Hz, 1H) 8.46 (s, 1H) 8.20 (d, J=8.90 Hz, 1H) 7.95 (s, 1H) 7.86-7.91 (m, 1H) 7.67 (t, J=8.27 Hz, 1H) 7.55 (d, J=10.81 Hz, 1H) 7.25-7.32 (m, 2H) 7.05 (d, J=5.72 Hz, 1H) 3.47 (br. s., 3H) 3.22-3.29 (m, 4H) 2.42 (br. s., 4H) 1.11 (s, 3H).

### Example 120: Synthesis of 2-((4-(4-(benzo[d]thiazol-5-ylamino)quinolin-6-yl)-3-fluorobenzyl)amino)-N-methylacetamide (Compound 320)

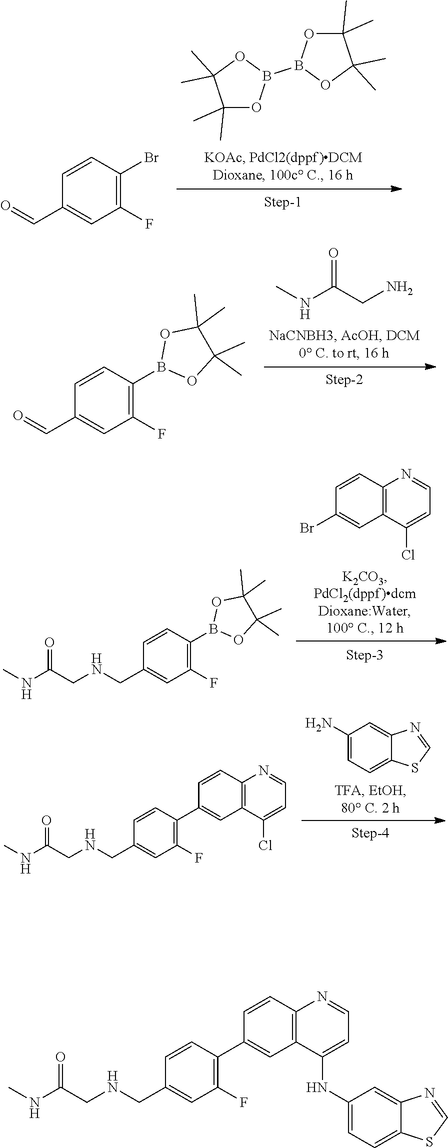

### Step-1: Synthesis of 3-fluoro-4-(4,4,5,5-tetramethyl-1,3,2-dioxaborolan-2-yl)benzaldehyde

To a solution of 4-bromo-3-fluorobenzaldehyde (2000 mg, 0.85 mmol, 1.0 equiv) in 1,4-dioxane (20 mL) was added KOAc (1930 mg, 19.70 mmol, 2.0 equiv) and 4,4,4′,4′,5,5,5′,5′-octamethyl-2,2′-bi(1,3,2-dioxaborolane) (563 mg, 2.21 mmol, 1.2 equiv). Reaction mixture was purged with nitrogen for 5 min, was added PdCl2dppf (402 mg, 0.49 mmol, 0.05 equiv). The reaction was heated at 100° C. for 16 h. The progress of reaction was monitored by TLC. After completion of reaction, reaction mixture was diluted with water (50 mL) and extracted with ethyl acetate (2×60 mL), dried over anhydrous sodium sulphate, filtered and concentrated under reduced pressure. The crude product was purified by flash chromatography (0 to 30% Ethyl acetate in hexane as an eluent) to afford 3-fluoro-4-(4,4,5,5-tetramethyl-1,3,2-dioxaborolan-2-yl)benzaldehyde (2300 mg, 90%), LCMS: 251.2 [M+1]⁺.

### Step-2: Synthesis of 2-((3-fluoro-4-(4,4,5,5-tetramethyl-1,3,2-dioxaborolan-2-yl)benzyl)amino)-N-methylacetamide

To a stirred solution 3-fluoro-4-(4,4,5,5-tetramethyl-1,3,2-dioxaborolan-2-yl)benzaldehyde (500 mg, 2.00 mmol, 1.0 equiv) and 2-amino-N-methylacetamide (353 mg, 4.00 mmol, 2.0 equiv) in DCM (5 mL) was added acetic acid (0.2 mL) at 0° C. Reaction mixture was stirred at RT for 1 h. NaCNBH₃ (189 mg, 3.00 mmol, 1.5 equiv) was added portion wise at 0° C. After addition, reaction mixture was stirred at RT for 16 h. The progress of reaction was monitored by LCMS. After completion of reaction, reaction mixture was diluted with water (30 mL) and was extracted with DCM (2×60 mL). The combined organic layer was washed with saturated NaHCO₃ solution (50 mL). The organic layer was dried over anhydrous sodium sulphate, filtered and concentrated under reduced pressure. to afford 2-((3-fluoro-4-(4,4,5,5-tetramethyl-1,3,2-dioxaborolan-2-yl)benzyl)amino)-N-methylacetamide (300 mg crude) directly used for next step without any purification.

### Step-3: Synthesis of 2-((4-(4-chloroquinolin-6-yl)-3-fluorobenzyl)amino)-N-methylacetamide

To a solution of 2-((3-fluoro-4-(4,4,5,5-tetramethyl-1,3,2-dioxaborolan-2-yl)benzyl)amino)-N-methylacetamide (600 mg, 1.86 mmol, 1.0 equiv) in 1,4-dioxane:water (10 mL:5 mL) was added K₂CO₃ (771 mg, 5.59 mmol, 2.0 equiv) and 6-bromo-4-chloroquinoline (631 mg, 2.61 mmol, 1.4 equiv). Reaction mixture was purged with nitrogen for 5 min, then was added PdCl₂dppf·DCM (76 mg, 0.09 mmol, 0.05 equiv). The reaction was heated at 100° C. for 16 h. The progress of reaction was monitored by TLC. After completion of reaction, reaction mixture was diluted with water (40 mL) and extracted with ethyl acetate (2×80 mL), dried over anhydrous sodium sulphate, filtered and concentrated under reduced pressure. The crude product was purified by flash chromatography (0 to 30% Ethyl acetate in hexane as an eluent) to afford 2-((4-(4-chloroquinolin-6-yl)-3-fluorobenzyl)amino)-N-methylacetamide (297 mg, 44%), LCMS: 358.1[M+1]⁺.

### Step-4: Synthesis of 2-((4-(4-(benzo[d]thiazol-5-ylamino)quinolin-6-yl)-3-fluorobenzyl)amino)-N-methylacetamide

To a solution of 2-((4-(4-chloroquinolin-6-yl)-3-fluorobenzyl)amino)-N-methylacetamide (290 mg, 0.81 mmol, 1.0 equiv) in ethanol (3 mL) was added benzo[d]thiazol-5-amine (122 mg, 0.81 mmol, 1.0 equiv) and TFA (0.2 mL). The reaction mixture was heated at 80° C. for 2 h. After completion of reaction, reaction mixture was concentrated under reduced pressure. The resulting residue was basified with saturated sodium bicarbonate solution (20 mL), was extracted with DCM (2×40 mL), dried over anhydrous sodium sulphate, filtered and concentrated under reduced pressure. The crude product was purified by reversed phase HPLC to afford 2-((4-(4-(benzo[d]thiazol-5-ylamino)quinolin-6-yl)-3-fluorobenzyl)amino)-N-methylacetamide (20 mg, 5%), LCMS: 472.2[M+1]^{+ 1}H NMR (400 MHz, DMSO-d₆) δ 9.43 (s, 1H), 9.27 (br. s., 1H), 8.60 (s, 1H), 8.50 (d, J=5.72 Hz, 1H), 8.18-8.29 (m, 2H), 8.03 (s, 1H), 7.97 (d, J=8.90 Hz, 1H), 7.89 (d, J=8.27 Hz, 1H), 7.82 (br. s., 1H), 7.67 (t, J=7.95 Hz, 1H), 7.55 (d, J=10.17 Hz, 1H), 7.25-7.49 (m, 3H), 7.05 (d, J=5.09 Hz, 1H), 2.59-2.74 (m, 7H).

### Example 121: Synthesis of (4-(4-(benzo[d]thiazol-5-ylamino)quinolin-6-yl)-3-fluorophenyl)(4-methoxypiperidin-1-yl)methanone (Compound 321)

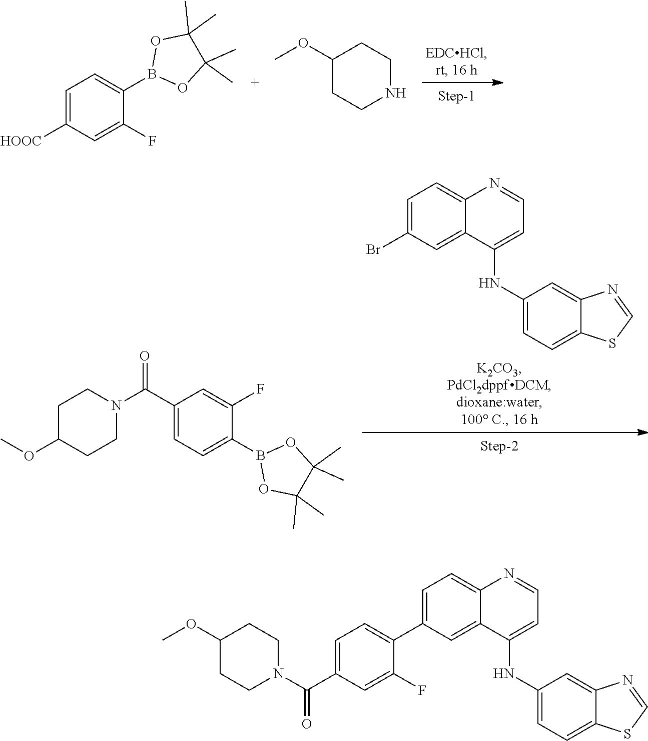

### Step-1: Synthesis of (3-fluoro-4-(4,4,5,5-tetramethyl-1,3,2-dioxaborolan-2-yl)phenyl)(4-methoxypiperidin-1-yl)methanone

To a solution of 3-fluoro-4-(4,4,5,5-tetramethyl-1,3,2-dioxaborolan-2-yl)benzoic acid (500 mg, 1.87 mmol, 1.0 equiv) in DCM (10 mL) was added EDC·HCl (466 mg, 2.43 mmol, 1.3 equiv) Reaction was stirred at RT for 1 h. Then was added 4-methoxypiperidine (260 mg, 2.25 mmol, 1.2 equiv). Reaction mixture was stirred at RT for 16 h. The progress of reaction was monitored by TLC. After completion of reaction, reaction mixture was diluted with water (30 mL) and extracted with ethyl acetate (2×60 mL). The combined organic layer was washed with water (20 mL×4) and brine solution (20 mL), dried over anhydrous sodium sulphate, concentrated under reduced pressure. The crude was purified by flash chromatography (0-70% ethyl acetate in hexane as an eluent) to afford (3-fluoro-4-(4,4,5,5-tetramethyl-1,3,2-dioxaborolan-2-yl)phenyl)(4-methoxypiperidin-1-yl)methanone (400 mg, 58%), LCMS: 282.4[boronic acid fragment]

### Step-2: Synthesis of (4-(4-(benzo[d]thiazol-5-ylamino)quinolin-6-yl)-3-fluorophenyl)(4-methoxypiperidin-1-yl)methanone

To a solution of (3-fluoro-4-(4,4,5,5-tetramethyl-1,3,2-dioxaborolan-2-yl)phenyl)(4-methoxypiperidin-1-yl)methanone (100 mg, 0.27 mmol, 1.0 equiv) in 1,4-dioxane:water (6 mL:2 mL) was added K₂CO₃ (94.8 mg, 0.68 mmol, 2.5 equiv) and N-(6-bromoquinolin-4-yl)benzo[d]thiazol-5-amine (118 mg, 0.33 mmol, 1.2 equiv). Reaction mixture was purged with nitrogen for 5 min, then was added PdCl₂dppf·DCM (11.2 mg, 0.01 mmol, 0.05 equiv). The reaction was heated at 100° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under reduced pressure. The crude product was purified by reversed phase HPLC to afford (4-(4-(benzo[d]thiazol-5-ylamino)quinolin-6-yl)-3-fluorophenyl)(4-methoxypiperidin-1-yl)methanone (10 mg, 7%), LCMS: 513.2[M+1]⁺. ¹H NMR (400 MHz, DMSO-d₆) δ 9.43 (s, 1H), 9.30 (br. s., 1H), 8.65 (br. s., 1H), 8.52 (d, J=5.1 Hz, 1H), 8.21 (d, J=8.9 Hz, 1H), 8.09-7.85 (m, 3H), 7.80 (t, J=7.6 Hz, 1H), 7.55 (d, J=8.3 Hz, 1H), 7.48-7.34 (m, 2H), 7.06 (d, J=4.5 Hz, 1H), 3.93 (br. s., 2H), 3.46 (br. s., 3H), 3.26 (br. s., 3H), 1.88 (br. s., 2H), 1.49 (br. s., 2H).

### Example 122: Synthesis of (4-(4-(benzo[d]thiazol-5-ylamino)-7-fluoroquinolin-6-yl)-3-fluorophenyl)(morpholino)methanone (Compound 322)

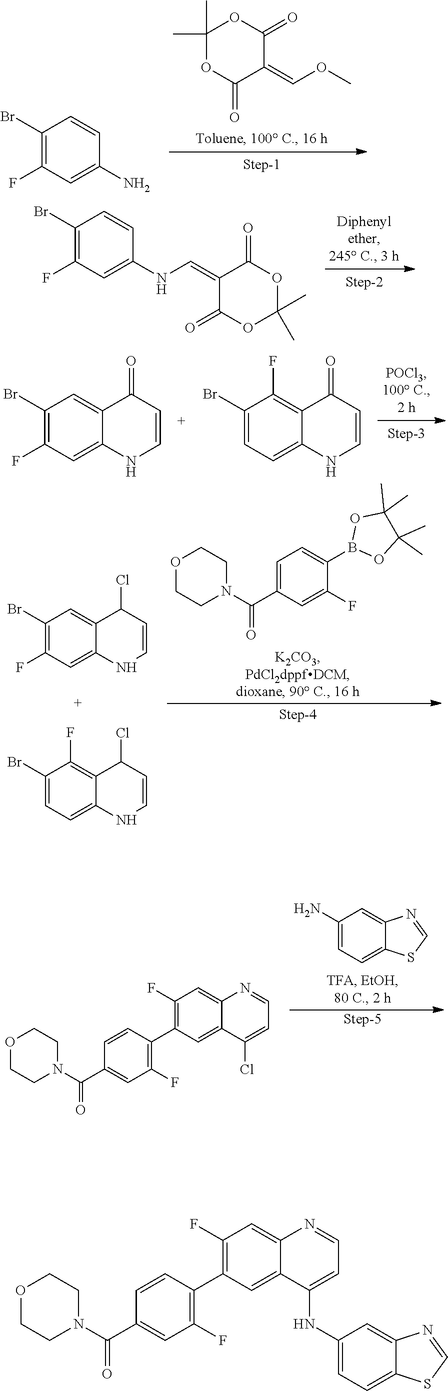

### Step-1: Synthesis of 5-((4-bromo-3-fluorophenylamino)methylene)-2,2-dimethyl-1,3-dioxane-4,6-dione

To a stirred solution of 5-(methoxymethylene)-2,2-dimethyl-1,3-dioxane-4,6-dione (4.0 g, 21.4 mmol, 1.0 equiv) in Toluene (25 mL), was added 4-bromo-3-fluoroaniline (4.08 mg, 21.4 mmol, 1.0 equiv). Reaction mixture was stirred at 100° C. for 16 h. The progress of the reaction was monitored by TLC and ¹H NMR. After completion of reaction, reaction mixture was concentrated under reduced pressure. The resulting residue was diluted with water (50 mL) and extracted with EtOAc (2×100 mL). Combined organic layers were dried over anhydrous Na₂SO₄, filtered and concentrated under reduced pressure to afford 5-((4-bromo-3-fluorophenylamino)methylene)-2,2-dimethyl-1,3-dioxane-4,6-dione (4.0 g, 55%) which was directly used for next step without any purification.

### Step-2: Synthesis of 6-bromo-7-fluoroquinolin-4(1H)-one and 6-bromo-5-fluoroquinolin-4(1H)-one

5-((4-bromo-3-fluorophenylamino)methylene)-2,2-dimethyl-1,3-dioxane-4,6-dione (4.0 g, 11.6 mmol, 1.0 equiv) in diphenyl ether (20 ml) was heated at 245° C. for 3 h. The progress of the reaction was monitored by LCMS and NMR. After completion of reaction, reaction mixture was allowed to cool at RT. Then the resulting solid product was washed diluted with hexane, filtered off and dried under vacuum to afford the mixture of 6-bromo-7-fluoroquinolin-4(1H)-one and 6-bromo-5-fluoroquinolin-4(1H)-one (2.5 g, 89%) LCMS: 242.2[M+1]⁺

### Step-3: Synthesis of 6-bromo-4-chloro-7-fluoroquinoline and 6-bromo-4-chloro-5-fluoroquinoline

Mixture of 6-bromo-7-fluoroquinolin-4(1H)-one and 6-bromo-5-fluoroquinolin-4(1H) (2.5 g, 10.3 mmol) in POCl3 (20 mL) was heated at 100° C. for 3 h. The progress of the reaction was monitored by LCMS and NMR. After completion of reaction, reaction mixture was quenched with sat. Sodium bicarbonate solution (50 mL), extracted with EtOAc (2×75 mL), combined organic layers were dried over Na2SO4, filtered and concentrated under reduced pressure. The resulting crude product was purified by flash chromatography (0-70% Ethyl acetate in hexane as an eluent) to afford mixture of 6-bromo-4-chloro-7-fluoroquinoline (1.2 g) LCMS: 262.2 [M+1]^{+ 1}H NMR (400 MHz, DMSO-d₆) δ 8.91 (d, J=5.09 Hz, 1H), 8.53 (d, J=7.63 Hz, 1H), 8.09 (d, J=9.54 Hz, 1H), 7.83 (d, J=5.09 Hz, 1H) and 6-bromo-4-chloro-5-fluoroquinoline (800 mg). LCMS: 262.2 [M+1]^{+ 1}H NMR (400 MHz, DMSO-d₆) δ 8.89 (d, J=5.09 Hz, 1H), 8.10 (dd, J=7.31, 9.22 Hz, 1H), 7.92 (d, J=8.27 Hz, 1H), 7.84 (d, J=5.09 Hz, 1H).

### Step-4: Synthesis of (4-(4-chloro-7-fluoroquinolin-6-yl)-3-fluorophenyl)(morpholino)methanone

To a solution of (3-fluoro-4-(4,4,5,5-tetramethyl-1,3,2-dioxaborolan-2-yl)phenyl)(morpholino)methanone (150 mg, 0.44 mmol, 1.0 equiv) in 1,4-dioxane:water (8.5 ml:1.5 ml) was added K₂CO₃ (151 mg, 1.10 mmol, 2.5 equiv) and 6-bromo-4-chloro-7-fluoroquinoline (140 mg, 0.53 mmol, 1.2 equiv). Reaction mixture was purged with nitrogen for 5 min, then was added PdCl₂dppf·DCM (18 mg, 0.02 mmol, 0.22 equiv). The reaction mixture was heated at 90° C. for 16 h. The progress of reaction was monitored by TLC. After completion of reaction, reaction mixture was diluted with water (50 mL) and extracted with ethyl acetate (2×100 mL), dried over anhydrous sodium sulphate, filtered and concentrated under reduced pressure. The crude which was purified by flash chromatography (0-5% MeOH in DCM as an eluent) to afford (4-(4-chloro-7-fluoroquinolin-6-yl)-3-fluorophenyl)(morpholino)methanone (100 mg, 57%), LCMS: 389.2 [M+1]⁺

### Step-5: Synthesis of (4-(4-(benzo[d]thiazol-5-ylamino)-7-fluoroquinolin-6-yl)-3-fluorophenyl)(morpholino)methanone

To a solution of (4-(4-chloro-7-fluoroquinolin-6-yl)-3-fluorophenyl)(morpholino)methanone (100 mg, 0.25 mmol, 1.0 equiv) and benzo[d]thiazol-5-amine (46.2 mg, 0.30 mmol, 1.2 equiv) in EtOH (5 mL) was added TFA (0.2 mL) and the reaction mixture was stirred at 80° C. for 2 h. The progress of the reaction was monitored by TLC and LCMS. After completion of reaction, reaction mixture was concentrated under reduced pressure to obtain crude which was basified with saturated NaHCO₃ solution (25 mL) and was extracted with DCM (2×50 mL). Combined organic layers were dried over Na2SO4, filtered and concentrated under reduced pressure. The crude was purified by flash chromatography, (0-6% MeOH:DCM as an eluent) to afford (4-(4-(benzo[d]thiazol-5-ylamino)-7-fluoroquinolin-6-yl)-3-fluorophenyl)(morpholino)methanone (30 mg, 23%). LCMS: 503.2[M+1]⁺, ¹H NMR (400 MHz, DMSO-d₆) δ 9.43 (s, 1H), 9.35 (s, 1H), 8.64 (s, 1H), 8.52 (d, J=5.7 Hz, 1H), 8.21 (d, J=8.9 Hz, 1H), 8.03 (s, 1H), 7.83-7.65 (m, 2H), 7.59-7.33 (m, 3H), 7.02 (d, J=5.1 Hz, 1H), 3.65 (br. s., 6H), 3.40 (br. s., 2H).

### Example 123: Synthesis of N-(7-(4-((2,6-diazaspiro[3.3]heptan-2-yl)methyl)-2-fluorophenyl)quinolin-4-yl)benzo[d]thiazol-5-amine (Compound 323)

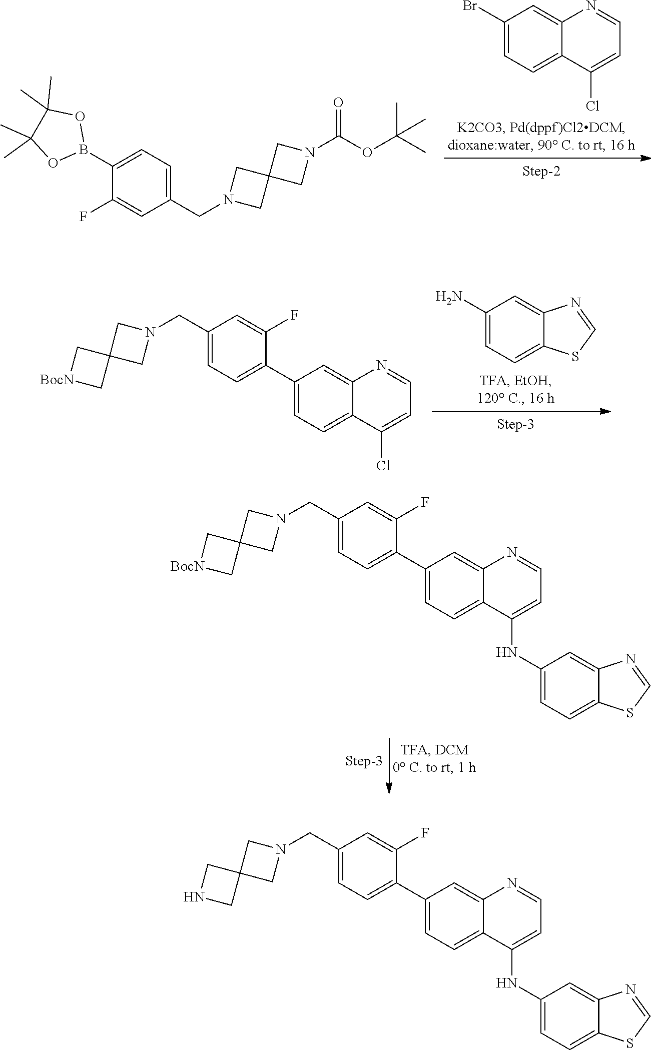

### Step-1: Synthesis of tert-butyl 6-(4-(4-chloroquinolin-7-yl)-3-fluorobenzyl)-2,6-diazaspiro[3.3]heptane-2-carboxylate

To a solution of tert-butyl 6-(3-fluoro-4-(4,4,5,5-tetramethyl-1,3,2-dioxaborolan-2-yl)benzyl)-2,6-diazaspiro[3.3]heptane-2-carboxylate (420 mg, 0.97 mmol, 1.0 equiv) in 1,4-dioxane:water (8 ml:2 ml) was added K₂CO₃ (268 mg, 1.94 mmol, 2.0 equiv) and 7-bromo-4-chloroquinoline (235 mg, 0.97 mmol, 1.0 equiv). Reaction mixture was purged with nitrogen for 5 min, then was added PdCl₂dppf·DCM (39 mg, 0.04 mmol, 0.05 equiv). The reaction was heated at 90° C. for 16 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure. The crude was purified by flash chromatography (0-5% MeOH in DCM as an eluent) to afford to afford tert-butyl 6-(4-(4-chloroquinolin-7-yl)-3-fluorobenzyl)-2,6-diazaspiro[3.3]heptane-2-carboxylate (220 mg, 48%), LCMS: 468.2 M+1]⁺.

### Step-2: Synthesis of tert-butyl 6-(4-(4-(benzo[d]thiazol-5-ylamino)quinolin-7-yl)-3-fluorobenzyl)-2,6-diazaspiro[3.3]heptane-2-carboxylate

To a solution of tert-butyl 6-(4-(4-chloroquinolin-7-yl)-3-fluorobenzyl)-2,6-diazaspiro[3.3]heptane-2-carboxylate (220 mg, 0.47 mmol, 1.0 equiv) in ethanol (10 mL) was added benzo[d]thiazol-5-amine (70 mg, 0.47 mmol, 1.0 equiv) and TFA (0.15 mL). The reaction mixture was heated at 100° C. for 2 h. After completion of reaction, reaction mixture was concentrated under reduced pressure. The residue was basified with saturated sodium bicarbonate solution (20 mL), was extracted with ethyl acetate (2×40 mL), dried over anhydrous sodium sulphate, filtered and concentrated under reduced pressure. The crude was purified by flash chromatography (0-8% MeOH in DCM as an eluent) to afford tert-butyl 6-(4-(4-(benzo[d]thiazol-5-ylamino)quinolin-7-yl)-3-fluorobenzyl)-2,6-diazaspiro[3.3]heptane-2-carboxylate (100 mg, 36%), LCMS: 582.2 [M+1]⁺.

### Step-3: Synthesis of N-(7-(4-((2,6-diazaspiro[3.3]heptan-2-yl)methyl)-2-fluorophenyl)quinolin-4-yl)benzo[d]thiazol-5-amine

To a solution of tert-butyl 6-(4-(4-(benzo[d]thiazol-5-ylamino)quinolin-7-yl)-3-fluorobenzyl)-2,6-diazaspiro[3.3]heptane-2-carboxylate (100 mg, 0.17 mmol, 1.0 equiv) in DCM (5 mL) was added TFA (1 mL) at 0° C. The reaction mixture was stirred at RT for 1 h. After completion of reaction, reaction mixture was concentrated under reduced pressure. The resulting residue was basified with saturated sodium bicarbonate solution (20 mL), was extracted with DCM (2×40 mL), dried over anhydrous sodium sulphate, filtered and concentrated under reduced pressure. The crude product was purified by flash column chromatography (0 to 10% MeOH in DCM as an eluent) to afford N-(7-(4-((2,6-diazaspiro[3.3]heptan-2-yl)methyl)-2-fluorophenyl)quinolin-4-yl)benzo[d]thiazol-5-amine (7 mg, 8%), LCMS: 482.3 [M+1]^{+1 1}H NMR (400 MHz, DMSO-d₆) δ 9.43 (s, 1H) 9.24 (s, 1H) 8.47-8.54 (m, 2H) 8.20 (d, J=8.27 Hz, 1H) 8.04 (s, 2H) 7.74 (d, J=8.27 Hz, 1H) 7.62-7.68 (m, 1H) 7.56 (d, J=6.36 Hz, 1H) 7.19-7.26 (m, 2H) 7.04 (d, J=5.09 Hz, 1H) 3.58 (m, 5H) 3.27 (m, 5H).

### Example 124: Synthesis of (4-(4-(benzo[d]thiazol-5-ylamino)quinolin-6-yl)-3-fluorophenyl)(2-oxa-6-azaspiro[3.3]heptan-6-yl)methanone (Compound 324)

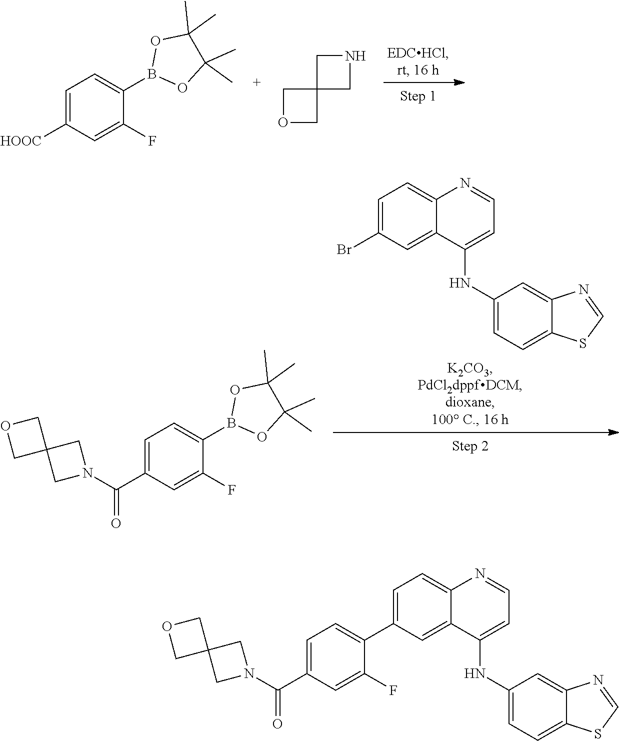 [SMILES: CC1(C)OB(c2ccc(C(=O)O)cc2F)OC1(C)C ; C1NCC12COC2 ; Brc1ccc2nccc(Nc3ccc4scnc4c3)c2c1 ; CC1(C)OB(c2ccc(C(=O)N3CC4(COC4)C3)cc2F)OC1(C)C ; O=C(c1ccc(-c2ccc3nccc(Nc4ccc5scnc5c4)c3c2)c(F)c1)N1CC2(COC2)C1]

### Step-1: Synthesis of (3-fluoro-4-(4,4,5,5-tetramethyl-1,3,2-dioxaborolan-2-yl)phenyl)(2-oxa-6-azaspiro[3.3]heptan-6-yl)methanone

To a solution of 3-fluoro-4-(4,4,5,5-tetramethyl-1,3,2-dioxaborolan-2-yl)benzoic acid (300 mg, 1.12 mmol, 1.0 equiv) in DCM (10 mL) was added EDC·HCl (280 mg, 1.46 mmol, 1.3 equiv) at RT and stirred for 1 h. Then was added, 2-oxa-6-azaspiro[3.3]heptane (134 mg, 1.35 mmol, 1.2 equiv). The resulting reaction mixture was stirred at RT for 16 h. The progress of reaction was monitored by TLC. After completion of reaction, reaction mixture was diluted with water (40 mL) and extracted with ethyl acetate (2×80 mL), washed with brine solution, dried over anhydrous sodium sulphate, filtered and concentrated under reduced pressure. The crude product was purified by flash chromatography (0 to 70% EtOAc in hexane as an eluent) to afford (3-fluoro-4-(4,4,5,5-tetramethyl-1,3,2-dioxaborolan-2-yl)phenyl)(2-oxa-6-azaspiro[3.3]heptan-6-yl)methanone (370 mg, 94%), LCMS: 266.4[boronic acid fragment]¹H NMR (400 MHz, DMSO-d₆) δ 7.70 (d, J=6.99 Hz, 1H) 7.43 (d, J=7.63 Hz, 1H) 7.32 (d, J=9.54 Hz, 1H) 4.67 (s, 4H) 4.47 (s, 2H) 4.21 (s, 2H) 1.30 (s, 12H).

### Step-2: Synthesis of (4-(4-(benzo[d]thiazol-5-ylamino)quinolin-6-yl)-3-fluorophenyl)(2-oxa-6-azaspiro[3.3]heptan-6-yl)methanone

To a solution of (3-fluoro-4-(4,4,5,5-tetramethyl-1,3,2-dioxaborolan-2-yl)phenyl)(2-oxa-6-azaspiro[3.3]heptan-6-yl)methanone (100 mg, 0.29 mmol, 1.0 equiv) in 1,4-dioxane:water (6 mL:2 mL) was added K₂CO₃ (79 mg, 0.58 mmol, 2.0 equiv) and N-(6-bromoquinolin-4-yl)benzo[d]thiazol-5-amine (102 mg, 0.29 mmol, 1.0 equiv). Reaction mixture was purged with nitrogen for 5 min, then was added PdCl₂dppf·DCM (11 mg, 0.01 mmol, 0.05 equiv). The reaction was heated at 100° C. for 16 h. The progress of reaction was monitored by TLC. After completion of reaction, reaction mixture was diluted with water (10 mL) and extracted with DCM (2×20 mL), dried over anhydrous sodium sulphate, filtered and concentrated under reduced pressure. The crude product was purified by reversed phase HPLC to afford (4-(4-(benzo[d]thiazol-5-ylamino)quinolin-6-yl)-3-fluorophenyl)(2-oxa-6-azaspiro[3.3]heptan-6-yl)methanone (11 mg, 8%), LCMS: 497.2[M+1]⁺. ¹H NMR (400 MHz, DMSO-d₆) δ 9.43 (s, 1H) 9.32 (s, 1H) 8.66 (s, 1H) 8.53 (d, J=5.09 Hz, 1H) 8.21 (d, J=8.27 Hz, 1H) 8.04 (d, J=1.91 Hz, 1H) 7.99 (d, J=8.27 Hz, 1H) 7.92 (d, J=8.90 Hz, 1H) 7.80-7.86 (m, 1H) 7.60-7.64 (m, 3H) 7.06 (d, J=5.09 Hz, 1H) 4.70 (s, 4H) 4.57 (s, 2H) 4.26 (s, 2H).

### Example 125: Synthesis of 4-(4-(benzo[d]thiazol-5-ylamino)quinolin-6-yl)-3-fluoro-N-(tetrahydro-2H-pyran-4-yl)benzamide (Compound 325)

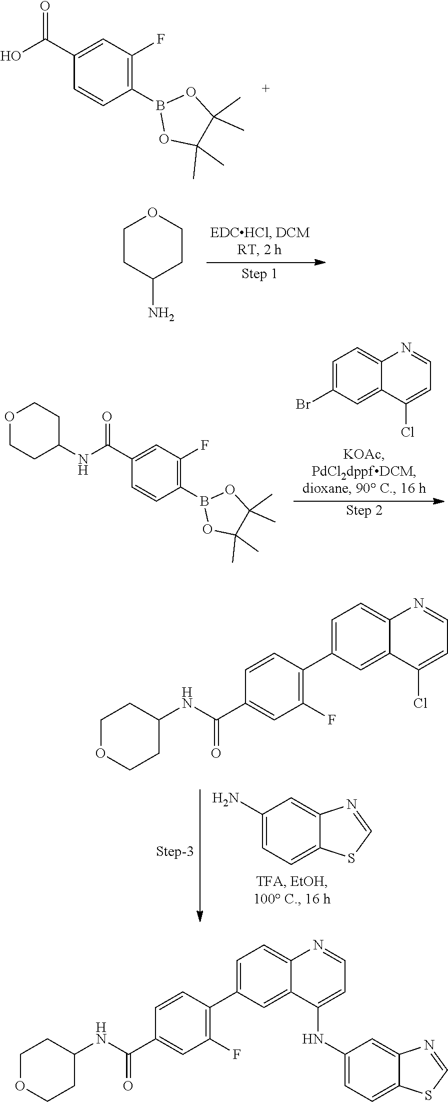

### Step-1: Synthesis of 3-fluoro-N-(tetrahydro-2H-pyran-4-yl)-4-(4,4,5,5-tetramethyl-1,3,2-dioxaborolan-2-yl)benzamide

To a solution of 3-fluoro-4-(4,4,5,5-tetramethyl-1,3,2-dioxaborolan-2-yl)benzoic acid (200 mg, 0.752 mmol, 1.0 equiv) in DCM (10 mL) was added EDC·HCl (173 mg, 0.90 mmol, 1.2 equiv) and stir for 10 min at rt. Tetrahydro-2H-pyran-4-amine (76 mg, 0.75 mmol, 1.0 equiv). The reaction mixture was stirred at RT for 2 h. The progress of reaction was monitored by TLC. After completion of reaction, reaction mixture was diluted with water (10 mL) and extracted with DCM (2×20 mL), washed with brine solution, dried over anhydrous sodium sulphate, filtered and concentrated under reduced pressure. The crude was purified by flash chromatography (0-30% ethyl acetate in hexane as an eluent) to afford 3-fluoro-N-(tetrahydro-2H-pyran-4-yl)-4-(4,4,5,5-tetramethyl-1,3,2-dioxaborolan-2-yl)benzamide (130 mg, 49%) as an off white solid. LCMS: 268.4 [M+1]⁺.[Boronic acid fragment]¹H NMR (400 MHz, DMSO-d₆) δ 8.46 (d, J=7.6 Hz, 1H), 7.79-7.66 (m, 2H), 7.61 (d, J=10.8 Hz, 1H), 4.06-3.96 (m, 1H), 3.94-3.72 (m, 2H), 3.45-3.34 (m, 2H), 1.82-1.67 (m, 2H), 1.57 (dq, J=4.5, 12.1 Hz, 2H), 1.35-1.28 (m, 12H).

### Step-2: Synthesis of 4-(4-chloroquinolin-6-yl)-3-fluoro-N-(tetrahydro-2H-pyran-4-yl)benzamide

To a solution of 3-fluoro-N-(tetrahydro-2H-pyran-4-yl)-4-(4,4,5,5-tetramethyl-1,3,2-dioxaborolan-2-yl)benzamide (60 mg, 0.17 mmol, 1.0 equiv) in 1,4-dioxane:water (5 mL:0.5 mL) was added K₂CO₃ (48 mg, 0.34 mmol, 2.0 equiv) and 6-bromo-4-chloroquinoline (42 mg, 0.17 mmol, 1.0 equiv). Reaction mixture was purged with nitrogen for 5 min, then was added PdCl₂dppf·DCM (7 mg, 0.009 mmol, 0.05 equiv). The reaction was heated at 90° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under reduced pressure. The crude was purified by flash chromatography (0-50% ethyl acetate in hexane as an eluent) to afford 4-(4-chloroquinolin-6-yl)-3-fluoro-N-(tetrahydro-2H-pyran-4-yl)benzamide (60 mg, 90%), LCMS: 385.2 [M+1]⁺. ¹H NMR (400 MHz, DMSO-d₆) δ 8.91 (d, J=4.5 Hz, 1H), 8.51 (d, J=7.6 Hz, 1H), 8.39 (s, 1H), 8.24 (d, J=8.3 Hz, 1H), 8.11 (d, J=8.9 Hz, 1H), 7.94-7.75 (m, 4H), 4.16-3.97 (m, 1H), 3.90 (d, J=10.8 Hz, 2H), 3.44-3.36 (m, 2H), 1.79 (d, J=12.1 Hz, 2H), 1.67-1.48 (m, 2H).

### Step-3: Synthesis of 4-(4-(benzo[d]thiazol-5-ylamino)quinolin-6-yl)-3-fluoro-N-(tetrahydro-2H-pyran-4-yl)benzamide

To a solution of 4-(4-chloroquinolin-6-yl)-3-fluoro-N-(tetrahydro-2H-pyran-4-yl)benzamide (60 mg, 0.16 mmol, 1.0 equiv) in ethanol (5 mL) was added benzo[d]thiazol-5-amine (24 mg, 0.16 mmol, 1.0 equiv) and TFA (0.1 mL). The reaction mixture was heated at 100° C. for 16 h. After completion of reaction, reaction mixture was concentrated under reduced pressure. The residue was basified with saturated sodium bicarbonate solution (20 mL), was extracted with ethyl acetate (2×40 mL), dried over anhydrous sodium sulphate, filtered and concentrated under reduced pressure. The crude was purified by flash chromatography (0-5% MeOH in DCM as an eluent) to afford 4-(4-(benzo[d]thiazol-5-ylamino)quinolin-6-yl)-3-fluoro-N-(tetrahydro-2H-pyran-4-yl)benzamide (11 mg, 14%) as an off white solid. LCMS: 499.3[M+1]⁺. ¹H NMR (400 MHz, DMSO-d₆) δ 9.44 (s, 1H), 9.39 (br. s., 1H), 8.68 (br. s., 1H), 8.58-8.39 (m, 2H), 8.22 (d, J=8.9 Hz, 1H), 8.08-7.81 (m, 6H), 7.56 (d, J=7.6 Hz, 1H), 7.05 (d, J=4.5 Hz, 1H), 4.04 (br. s., 1H), 3.90 (d, J=9.5 Hz, 2H), 3.50-3.37 (m, 2H), 1.79 (d, J=11.4 Hz, 2H), 1.61 (d, J=8.3 Hz, 2H).

### Example 126: Synthesis of (5-(4-(benzo[d]thiazol-5-ylamino)quinolin-6-yl)pyridin-2-yl)(morpholino)methanone (Compound 326)

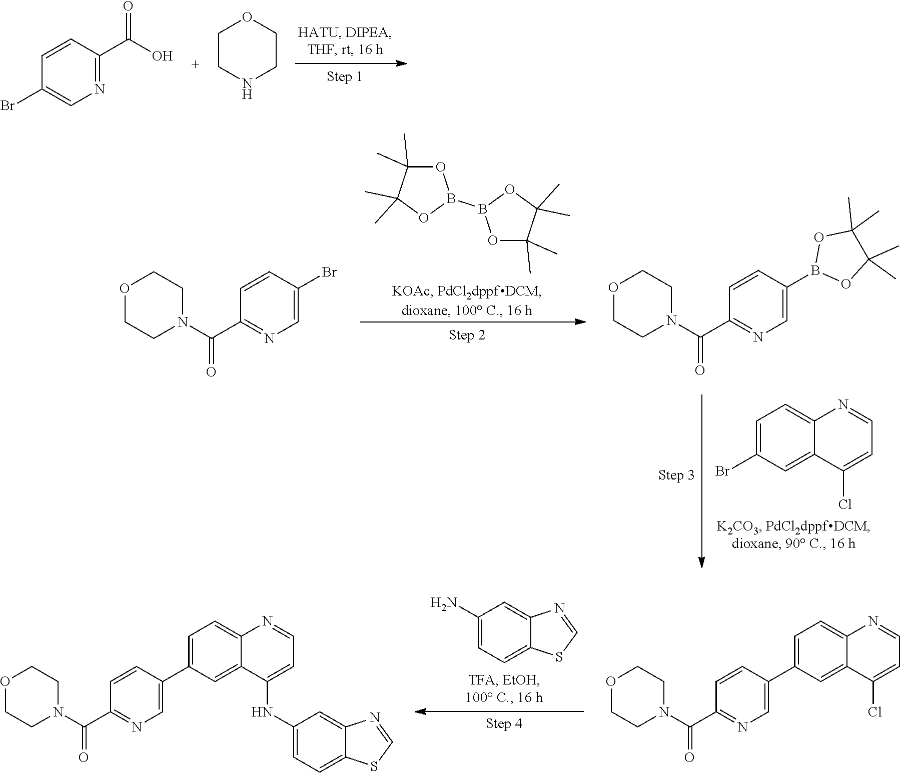

### Step-1: Synthesis of (5-bromopyridin-2-yl)(morpholino)methanone

To a solution of 5-bromopicolinic acid (500 mg, 2.48 mmol, 1.0 equiv) in DMF (5 mL) was added HATU (227 mg, 2.97 mmol, 1.2 equiv), and morpholine (215 mg, 2.48 mmol, 1.0 equiv), followed by the addition of DIPEA (1.33 mL, 7.43 mmol, 3.0 equiv). The reaction mixture was stirred at RT for 16 h. The progress of reaction was monitored by TLC. After completion of reaction, reaction mixture was diluted with water (30 mL) and extracted with ethyl acetate (2×60 mL). The combined organic layer was washed with water (20 mL×4) and brine solution (20 mL), dried over anhydrous sodium sulphate, concentrated under reduced pressure. The crude was purified by flash chromatography (0-30% ethyl acetate in hexane as an eluent) to afford (5-bromopyridin-2-yl)(morpholino)methanone (650 mg, 96%), LCMS: 271.3 [M+1]^{+ 1} H NMR (400 MHz, DMSO-d₆) δ 8.73 (d, J=1.9 Hz, 1H), 8.20 (dd, J=2.2, 8.6 Hz, 1H), 7.59 (d, J=8.3 Hz, 1H), 3.65 (d, J=4.5 Hz, 4H), 3.58-3.51 (m, 2H), 3.51-3.36 (m, 2H).

### Step-2: Synthesis of morpholino(5-(4,4,5,5-tetramethyl-1,3,2-dioxaborolan-2-yl)pyridin-2-yl)methanone

To a solution (5-bromopyridin-2-yl)(morpholino)methanone (500 mg, 1.84 mmol, 0.01 equiv) in 1,4-dioxane (20 mL) was added KOAc (361 mg, 3.69 mmol, 2.0 equiv) and 4,4,4′,4′,5,5,5′,5′-octamethyl-2,2′-bi(1,3,2-dioxaborolane) (562 mg, 2.21 mmol, 1.2 equiv). Reaction mixture was purged with nitrogen for 5 min, then was added PdCl₂dppf·DCM (75 mg, 0.09 mmol, 0.05 equiv). The reaction mixture was heated at 100° C. for 16 h. The progress of reaction was monitored by TLC. After completion of reaction, reaction mixture was filtered from Celite® and filtrate concentrated under vacuum to afford morpholino(5-(4,4,5,5-tetramethyl-1,3,2-dioxaborolan-2-yl)pyridin-2-yl)methanone (500 mg, 98%), LCMS: 237.4 [M+1]⁺.[Boronic acid fragment].

### Step-3: Synthesis of (5-(4-chloroquinolin-6-yl)pyridin-2-yl)(morpholino)methanone

To a solution of morpholino(5-(4,4,5,5-tetramethyl-1,3,2-dioxaborolan-2-yl)pyridin-2-yl)methanone (250 mg, 0.79 mmol, 1.0 equiv) in 1,4-dioxane:water (10 mL:0.1 mL) was added K₂CO₃ (215 mg, 1.56 mmol, 2.0 equiv) and 6-bromo-4-chloroquinoline (152 mg, 0.63 mmol, 0.8 equiv). Reaction mixture was purged with nitrogen for 5 min, then was added PdCl₂dppf·DCM (32 mg, 0.04 mmol, 0.05 equiv). The reaction was heated at 90° C. for 16 h. The progress of reaction was monitored by TLC. After completion of reaction, reaction mixture was diluted with water (20 mL) and extracted with ethyl acetate (2×40 mL), dried over anhydrous sodium sulphate, filtered and concentrated under reduced pressure. The crude was purified by flash chromatography (0-50% ethyl acetate in hexane as an eluent) to afford (5-(4-chloroquinolin-6-yl)pyridin-2-yl)(morpholino)methanone (120 mg, 43%), LCMS: 354.3[M+1]⁺. ¹H NMR (400 MHz, DMSO-d₆) δ 9.10 (d, J=1.9 Hz, 1H), 8.90 (d, J=5.1 Hz, 1H), 8.50 (d, J=1.9 Hz, 1H), 8.43 (dd, J=2.5, 8.3 Hz, 1H), 8.36-8.21 (m, 2H), 7.85 (d, J=4.5 Hz, 1H), 7.78 (d, J=8.3 Hz, 1H), 3.70 (s, 4H), 3.57 (dd, J=4.8, 19.4 Hz, 4H).

### Step-4: Synthesis of (5-(4-(benzo[d]thiazol-5-ylamino)quinolin-6-yl)pyridin-2-yl)(morpholino)methanone

To a solution of (5-(4-chloroquinolin-6-yl)pyridin-2-yl)(morpholino)methanone (80 mg, 0.23 mmol, 1.0 equiv) in ethanol (8 mL) was added benzo[d]thiazol-5-amine (34 mg, 0.23 mmol, 1.0 equiv) and TFA (0.1 mL). The reaction mixture was heated at 100° C. for 16 h. After completion of reaction, reaction mixture was concentrated under reduced pressure. The residue was basified with saturated sodium bicarbonate solution (20 mL), was extracted with ethyl acetate (2×40 mL), dried over anhydrous sodium sulphate, filtered and concentrated under reduced pressure. The crude was purified by flash chromatography (0-5% MeOH in DCM as an eluent) to afford (5-(4-(benzo[d]thiazol-5-ylamino)quinolin-6-yl)pyridin-2-yl)(morpholino)methanone (7 mg, 7%), LCMS: 468.3[M+1]⁺. ¹H NMR (400 MHz, DMSO-d₆) δ 9.47-9.32 (m, 2H), 9.28-9.15 (m, 1H), 8.90 (s, 1H), 8.56-8.35 (m, 2H), 8.30-8.11 (m, 2H), 8.02 (d, J=8.3 Hz, 1H), 7.80 (d, J=7.6 Hz, 1H), 7.58 (d, J=8.3 Hz, 1H), 7.04 (d, J=5.1 Hz, 1H), 3.70 (s, 4H), 3.67-3.54 (m, 4H).

### Example 127: Synthesis of 4-(4-(4-(benzo[d]thiazol-5-ylamino)quinolin-7-yl)-3-fluorobenzyl)-1-methylpiperazin-2-one (Compound 327)

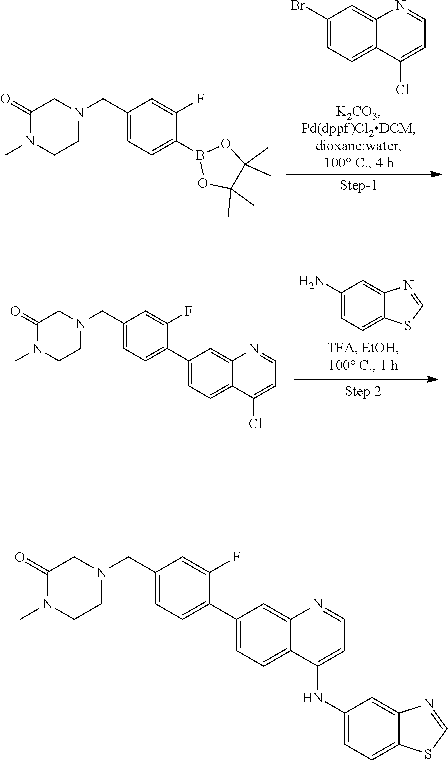 [SMILES: Clc1ccnc2cc(Br)ccc12 ; CN1CCN(Cc2ccc(B3OC(C)(C)C(C)(C)O3)c(F)c2)CC1=O ; CN1CCN(Cc2ccc(-c3ccc4c(Cl)ccnc4c3)c(F)c2)CC1=O ; Nc1ccc2scnc2c1 ; CN1CCN(Cc2ccc(-c3ccc4c(Nc5ccc6scnc6c5)ccnc4c3)c(F)c2)CC1=O]

### Step-1: Synthesis of 4-(4-(4-chloroquinolin-7-yl)-3-fluorobenzyl)-1-methylpiperazin-2-one

To a solution of 4-(3-fluoro-4-(4,4,5,5-tetramethyl-1,3,2-dioxaborolan-2-yl)benzyl)-1-methylpiperazin-2-one (270 mg, 0.77 mmol, 1.0 equiv) in 1,4-dioxane:water (8 ml:2 ml) was added K₂CO₃ (214 mg, 1.55 mmol, 2.0 equiv) and 7-bromo-4-chloroquinoline (188 mg, 0.77 mmol, 1.0 equiv). Reaction mixture was purged with nitrogen for 5 min, then was added PdCl₂dppf·DCM (31 mg, 0.03 mmol, 0.05 equiv). The reaction was heated at 100° C. for 4 h. The progress of reaction was monitored by TLC. After completion of reaction, reaction mixture was diluted with water (30 mL) and extracted with ethyl acetate (2×60 mL), dried over anhydrous sodium sulphate, filtered and concentrated under reduced pressure. The crude was purified by flash chromatography (0-5% MeOH in DCM as an eluent) to afford to afford 4-(4-(4-chloroquinolin-7-yl)-3-fluorobenzyl)-1-methylpiperazin-2-one (145 mg, 49%), LCMS: 384.3 M+1]⁺.

### Step-2: Synthesis of 4-(4-(4-(benzo[d]thiazol-5-ylamino)quinolin-7-yl)-3-fluorobenzyl)-1-methylpiperazin-2-one

To a solution of 4-(4-(4-chloroquinolin-7-yl)-3-fluorobenzyl)-1-methylpiperazin-2-one (145 mg, 0.38 mmol, 1.0 equiv) in ethanol (10 mL) was added benzo[d]thiazol-5-amine (56 mg, 0.38 mmol, 1.0 equiv) and TFA (0.15 mL). The reaction mixture was heated at 100° C. for 1 h. After completion of reaction, reaction mixture was concentrated under reduced pressure. The residue was basified with saturated sodium bicarbonate solution (20 mL), was extracted with ethyl acetate (2×40 mL), dried over anhydrous sodium sulphate, filtered and concentrated under reduced pressure. The crude was purified by flash chromatography (0-8% MeOH in DCM as an eluent) to afford 4-(4-(4-(benzo[d]thiazol-5-ylamino)quinolin-7-yl)-3-fluorobenzyl)-1-methylpiperazin-2-one (85 mg, 45%), LCMS: 498.3[M+1]⁺. ¹H NMR (400 MHz, DMSO-d₆) δ 9.43 (s, 1H) 9.26 (br. s., 1H) 8.49-8.54 (m, 2H) 8.20 (d, J=8.90 Hz, 1H) 8.06 (d, J=5.09 Hz, 2H) 7.77 (d, J=8.90 Hz, 1H) 7.71 (t, J=8.27 Hz, 1H) 7.56 (d, J=8.27 Hz, 1H) 7.30-7.36 (m, 2H) 7.04 (d, J=5.09 Hz, 1H) 3.64 (s, 2H) 3.29 (br. s., 2H) 3.03 (s, 2H) 2.84 (s, 3H) 2.69 (dd, J=12.08, 6.36 Hz, 2H).

### Biological Example 1. RIPK3 Kinase Assay

The assays were performed in Reaction Biology Corporation (RBC), Malvern, USA. Compounds were diluted with DMSO to a final stock concentration of 10 mM and then were tested in a 10-dose IC₅₀ mode with a 3-fold serial dilution starting at 10 PM. All the reactions were carried out at 10 μM ATP and MBP 20 μM as substrates. RIPK3 activity was determined using radiometric HotSpot™ kinase assay protocol and IC₅₀ values were calculated use GraphPad Prism software. IC₅₀ levels for the inhibition of RIPK3 activity are shown in Table 2 (NT represents compound not tested). Compound A is N-(6-(isopropylsulfonyl)quinolin-4-yl)benzo[d]thiazol-5-amine (WO2011140442):

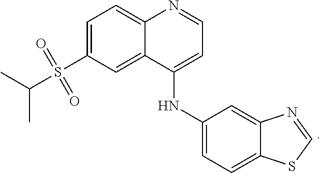 [SMILES: CC(C)S(=O)(=O)c1ccc2nccc(Nc3ccc4scnc4c3)c2c1]

TABLE 2

| Compound | RIPK3 kinase assay |
|---|---|
| Nº | IC₅₀ (nM) |
| Compound A | 2.01 |
| 1 | 2.58 |
| 11 | 1.29 |
| 12 | 1.67 |
| 13 | 1.72 |
| 14 | 2.99 |
| 15 | 3.57 |
| 16 | 0.51 |
| 17 | 0.95 |
| 21 | 1.51 |
| 22 | 2.13 |
| 23 | 2.21 |
| 27 | 1.13 |
| 28 | 3.47 |
| 29 | 2.31 |
| 31 | 2.75 |
| 34 | 0.65 |
| 35 | 3.97 |
| 36 | 1.28 |
| 37 | 1.79 |
| 40 | 18.30 |
| 41 | 0.133 |
| 43 | 0.162 |
| 47 | 0.86 |
| 50 | 0.102 |
| 51 | 1.88 |
| 52 | 0.57 |
| 53 | 0.72 |
| 54 | 0.308 |
| 61 | 0.029 |
| 244 | 1.92 |
| 249 | 1.75 |
| 276 | 6.89 |
| 284 | 1.13 |
| 285 | 3.14 |
| 292 | 3.67 |

### Biological Example 2. Necroptosis Inhibition Assay

Murine L929 cells (ATCC-CCL-1) were seeded at 10,000 cells/well in a 96-well plate. Cells were treated 48 hours after seeding with 10 ng/ml mTNFα (R&D Systems) and 20 μM zVAD (ChemShuttle) in the presence of different concentrations of compounds ranging from 10 M to 0.01 μM. Cells treated with 0.1% DMSO or cells treated with TNFα+zVAD were used as control and vehicle respectively. Compound A is N-(6-(isopropylsulfonyl)quinolin-4-yl)benzo[d]thiazol-5-amine (WO2011140442):

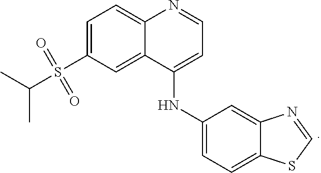 [SMILES: CC(C)S(=O)(=O)c1ccc2nccc(Nc3ccc4scnc4c3)c2c1]

Assay plates containing cells were incubated for 6 hours at 37° C. and 5% CO₂. Necroptosis was assessed using Sytox Green Nucleic Acid Stain (Invitrogen). After incubation, 100 μL of Sytox dye at 2 μM (2×) diluted in HBSS 1× was added in each well and incubated for 15 min at room temperature. Fluorescence (500ₑₓ/530ₑₘ) was recorded using the Cytation 5 microplate reader (Biotek).

Human HT-29 cells (ATCC-HTB-38) were seeded at 10,000 cells/well in a 96-well plate. Cells were treated 48 hours after seeding with 10 ng/ml hTNFα (R&D Systems), 20 μM zVAD (ChemShuttle) and 100 nM SMAC mimetic (MedChem Express) in the presence of different concentrations of compounds ranging from 10 M to 0.01 μM. Cells treated with 0.1% DMSO or cells treated with TNFα+zVAD+SMAC mimetic were used as control and vehicle respectively.

Assay plates containing cells were incubated for 6 hours at 37° C. and 5% CO₂. Necroptosis was assessed using Sytox Green Nucleic Acid Stain (Invitrogen) and Hoechst staining. After incubation, 100 μL of Sytox dye at 2 μM (2×) diluted in HBSS 1× was added to each well. At the same time, Hoechst stain is added to a final concentration of 10 μM and incubated for 15 min at room temperature. Images were taken for Sytox and Hoechst using the Cytation 5 microplate reader with DAPI and GFP filter cubes (Biotek). Sytox and Hoechst positive cells were counted with CellProfiler software.

Percentage of necroptosis inhibition was calculated as the percent compared to control and vehicle conditions which correspond to 100% and 0% respectively. The half-maximum necroptosis inhibition (IC₅₀) levels were calculated using GraphPad Prism software and are shown in Table 3 (NT represents compound not tested).

TABLE 3

| Compound | L929 necroptosis | HT29 necroptosis |
|---|---|---|
| Nº | inhibition IC₅₀ (nM) | inhibition IC₅₀ (nM) |
| Compound A | 1,963 | 1,263.0 |
| 1 | 341.3 | 333.8 |
| 2 | >10,000 | NT |
| 3 | >10,000 | NT |
| 4 | >10,000 | NT |
| 5 | >10,000 | NT |
| 6 | >10,000 | NT |
| 7 | >10,000 | NT |
| 8 | >10,000 | NT |
| 9 | >10,000 | NT |
| 10 | >10,000 | NT |
| 11 | 332.7 | 102.3 |
| 12 | 776.5 | 207.8 |
| 13 | 1,133 | 316.8 |
| 14 | 491.6 | 565.4 |
| 15 | 1,190 | >1,000 |
| 16 | 104.5 | 157.8 |
| 17 | 341.1 | 97.1 |
| 18 | <10,000 | NT |
| 19 | >10,000 | NT |
| 20 | <10,000 | NT |
| 21 | <10,000 | NT |
| 22 | 315.9 | 327.0 |
| 23 | 274.7 | 302.2 |
| 24 | <10,000 | NT |
| 25 | >10,000 | NT |
| 26 | >10,000 | NT |
| 27 | 669.3 | 167.6 |
| 28 | 2,589 | >10,000 |
| 29 | 494.3 | 341.1 |
| 30 | >10,000 | NT |
| 31 | 1,658 | 522.9 |
| 32 | >10,000 | NT |
| 33 | <10,000 | NT |
| 34 | 68.5 | 118.7 |
| 35 | 818.4 | 116.2 |
| 36 | 318.0 | 352.8 |
| 37 | 444.8 | 669.7 |
| 38 | <10,000 | NT |
| 39 | >10,000 | NT |
| 40 | 1,060 | 7874 |
| 41 | 364.8 | 86.8 |
| 42 | <10,000 | NT |
| 43 | 187.5 | 93.9 |
| 44 | <10,000 | NT |
| 47 | 420.6 | 1,015 |
| 50 | 320.4 | 707.5 |
| 51 | 832.3 | 1,203 |
| 52 | 322.7 | 505.3 |
| 53 | 513.3 | 704.7 |
| 54 | 200.6 | 630.4 |
| 55 | <10,000 | NT |
| 56 | >10,000 | NT |
| 57 | >10,000 | NT |
| 58 | <10,000 | NT |
| 61 | 93.3 | 94.2 |
| 239 | <1,000 | NT |
| 240 | >1,000 | NT |
| 241 | 387.6 | NT |
| 242 | <1,000 | NT |
| 243 | <1,000 | NT |
| 244 | <1,000 | NT |
| 245 | <1,000 | NT |
| 246 | <1,000 | NT |
| 247 | >1,000 | NT |
| 248 | <1,000 | NT |
| 249 | 1,259.0 | 67.9 |
| 250 | 344.1 | 2,531 |
| 251 | 582.0 | 822.5 |
| 252 | 260.0 | 244.4 |
| 253 | 271.1 | 755.3 |
| 256 | >10,000 | NT |
| 271 | >10,000 | NT |
| 273 | 1,103.0 | 780.1 |
| 274 | 514.9 | 1,187 |
| 275 | 3,819.0 | 688.9 |
| 276 | 552.9 | 538.5 |
| 281 | 436.4 | 202.6 |
| 282 | <10,000 | NT |
| 283 | 321.5 | 234.9 |
| 284 | 959.7 | 385.5 |
| 285 | 2,711.0 | 129.2 |
| 286 | 852.5 | 75.8 |
| 287 | >10,000 | NT |
| 288 | >10,000 | NT |
| 289 | >10,000 | NT |
| 290 | <10,000 | <1,000 |
| 291 | <10,000 | <1,000 |
| 292 | 1,780 | 159.2 |
| 293 | 347.3 | 32.3 |
| 294 | >10,000 | NT |
| 295 | 781.3 | 147.4 |
| 296 | 1,292.0 | 16.9 |
| 297 | 282.2 | 173.5 |
| 298 | <10,000 | NT |
| 299 | 987.2 | 461.6 |
| 300 | 461.2 | 721.2 |
| 301 | 426.7 | 196.8 |
| 302 | <10,000 | NT |
| 303 | 977.0 | 689.9 |
| 304 | <10,000 | NT |
| 305 | > 10,000 | 401.8 |
| 306 | <10,000 | 148.3 |
| 307 | 773.6 | 271.3 |
| 308 | <10,000 | NT |
| 309 | <10,000 | NT |
| 310 | 662.6 | 294.5 |
| 311 | 1030 | 732.1 |
| 312 | 668.3 | 1,557.0 |
| 313 | 401.9 | 302.3 |
| 314 | <10,000 | NT |
| 315 | 396.1 | 120.0 |
| 316 | 1,576 | 269.1 |
| 317 | 707.3 | 168.8 |
| 318 | 685.6 | 53.7 |
| 319 | 300.7 | 283.9 |
| 320 | 444.1 | 242.6 |
| 321 | 237.9 | 109.2 |
| 322 | 175.2 | 111.3 |
| 323 | 453.7 | 296.4 |
| 324 | 223.4 | 76.5 |
| 325 | 664.6 | 69.7 |
| 326 | 1,790 | 305.3 |
| 327 | 160.3 | 25.2 |
| 385 | >10,000 | NT |
| 386 | >10,000 | NT |
| 387 | >10,000 | NT |
| 388 | >10,000 | NT |

### Biological Example 3. Neuronal Degeneration In Vitro

Primary cultures of sensory neurons derived from embryonic dorsal root ganglia (DRGs) represents an excellent in vitro model to test interventions that can inhibit neuronal degeneration triggered by different stimuli, including mechanical insults and chemotherapy-induced axonal degeneration.

Sensory neurons are obtained from dorsal root ganglia of embryonic day 14.5 mice embryos which are cultured in 24-well dishes containing 400 μl of Neurobasal medium (Gibco, 21103-049), 2% B27 (Gibco, 17504-044), 0.3% 1-glutamine (Gibco, 25030-081), 1% streptomycin/penicillin (Gibco, 15240-062), 4 M aphidicolin (Sigma-Aldrich, A0781), 7.5 g/ml 5-fluoro-2-deoxyuridine (Sigma-Aldrich, F0503), and 50 ng/ml nerve growth factor (Invitrogen, 13257-019). The mixture of aphidicolin and fluoro-2-deoxyuridine effectively suppressed Schwann cell proliferation by inhibiting DNA polymerase, thus yielding a highly enriched sensory neuron culture.

For activation of neurodegeneration primary sensory neurons are exposed to 100 M oxaliplatin for different time periods in the presence of different concentrations of compounds ranging from 10 M to 0.01 M. Cells treated with 0.1% DMSO are used as vehicle. Mechanical injury is performed by elimination of the neuronal somas, leaving injured axons in culture for different time periods in the presence of different concentrations of compounds ranging from 10 M to 0.01 M. Cells treated with 0.1% DMSO are used as vehicle.

The efficacy of compound treatments on neuronal degeneration in sensory neurons can be measured.

For the sake of brevity, the disclosures of publications cited in this specification, including patents, are herein incorporated by reference.

What is claimed is:

1. A compound of formula (Ia): 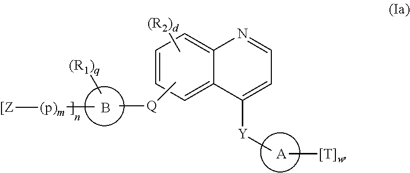
or a pharmaceutically acceptable salt, stereoisomer, or tautomer thereof, wherein:
d is an integer from 0 to 3;
m is an integer from 0 to 2;
n is an integer from 0 to 5;
q is an integer from 0 to 4;
w is an integer from 0 to 9;
Q is a bond or —NH—;
Ring B is C₆₋₁₄ aryl or pyridyl;
each R¹ is independently halo, —CN, C₁₋₆ alkyl or C₁₋₆ alkoxy;
each R² is independently halo;
each p is independently selected from the group consisting of —O—, C₁₋₆ alkylene, —NR^{a}—, —C(O)—, #—NR^{a}SO₂—, #—R^{a}OR^{b}—, #—C(O)NR^{a}—, #—C(O)NR^{a}R^{b}—, #—C(O)R^{a}—, #—C(O)OR^{a}, and #—OR^{a}—, wherein #indicates the point of attachment to Ring B;
each of R^{a} and R^{b} is independently H, C₁₋₆ alkyl, C₁₋₆ alkylene, or —C(O)C₁₋₆ alkyl;
each Z is independently —NR^{c}R^{d}, 3- to 12-membered heterocyclyl optionally substituted with one or more R^{Za}, 5- to 12-membered heteroaryl optionally substituted with one or more R^{Zb}, C₆₋₁₄ aryl optionally substituted with one or more R^{Zc}, or 3- to 12-membered cycloalkyl optionally substituted with one or more R^{Zd};
each R^{Za}, R^{Zb}, R^{Zc}, and R^{Zd} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, —SO₂R^{c}—, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, R^{Zc}, and R^{Zd} is optionally substituted with one or more halo, —OH, C₁₋₆ alkyl, —C(O)OR^{c}, or —C(O)NR^{c}R^{d};
each R^{c} and R^{d} is independently H, —NH₂, or C₁₋₆ alkyl optionally substituted with one or more —OH, halo, —NH₂, —NHC₁₋₆ alkyl, —CF₃, or oxo;
Y is —NH— or —O—;
Ring A is selected from the group consisting of: 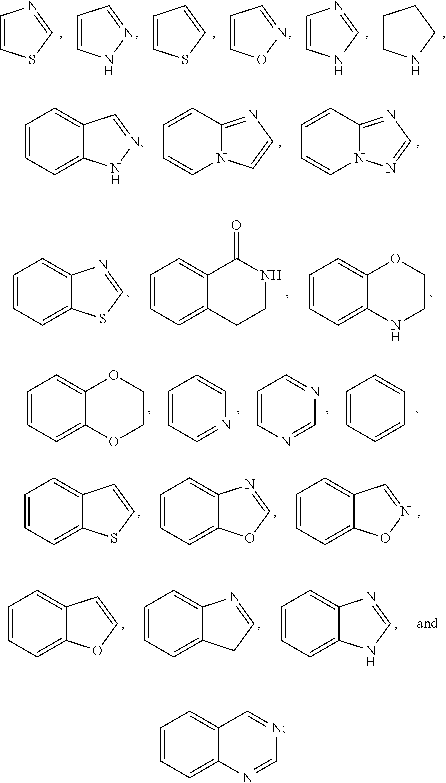
and
each T is independently selected from the group consisting of oxo, halo, —NH₂, C₁-C₆ alkyl, C₁₋₆ alkoxy, —NHC(O)—C₃₋₆cycloalkyl, and methylpiperidyl, provided that:
(1) n+w is an integer greater than 0,
(2) when Ring A is phenyl, then either: (a) n is an integer from 1 to 5 and Ring B is substituted by at least one (p)ₘ-Z wherein Z is 3- to 12-membered heterocyclyl substituted with one or more R^{Za} or (b) Ring A is substituted by at least one T wherein T is methylpiperidyl, and
(3) when Ring B is phenyl, m is 0, and Z is piperazinyl, at least one R^{Za} is present and is CH₃ or oxo.

2. A compound of formula (I): 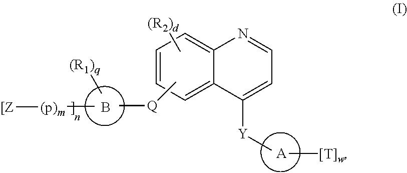
or a pharmaceutically acceptable salt, stereoisomer, or tautomer thereof, wherein:
d is an integer from 0 to 3;
m is an integer from 0 to 2;
n is an integer from 0 to 5;
q is an integer from 0 to 4;
w is an integer from 0 to 9;
Q is a bond or —NH—;
Ring B is C₆₋₁₄ aryl or pyridyl;
each R¹ is independently halo, —CN, C₁₋₆ alkyl or C₁₋₆ alkoxy;
each R² is independently halo;
each p is independently selected from the group consisting of —O—, C₁₋₆ alkylene, —NR^{a}—, —C(O)—, #—NR^{a}SO₂—, #—R^{a}OR^{b}—, #—C(O)NR^{a}R^{b}—, #—C(O)R^{a}—, #—C(O)OR^{a}, and #—OR^{a}—, wherein #indicates the point of attachment to Ring B;
each of R^{a} and R^{b} is independently H, C₁₋₆ alkyl or C₁₋₆ alkylene;
each Z is independently —NR^{c}R^{d}, 3- to 12-membered heterocyclyl optionally substituted with one or more R^{Za}, 5- to 12-membered heteroaryl optionally substituted with one or more R^{Zb}, or C₆₋₁₄ aryl optionally substituted with one or more R^{Zc};
each R^{Za}, R^{Zb}, and R^{Zc} is independently selected from the group consisting of oxo, halo, —OH, O⁻, —NR^{a}R^{b}, C₁₋₆ alkyl, C₁₋₆alkoxy, —C(O)OR^{c}, —C(O)R^{c}, C₃-C₁₂ cycloalkyl, 4- to 12-membered heterocyclyl, C₆₋₁₄ aryl, and 5- to 12-membered heteroaryl, wherein each C₁₋₆ alkyl, C₁₋₆alkoxy, C₃-C₁₂ cycloalkyl, 4- to 12-membered heterocyclyl, C₆₋₁₄ aryl, and 5- to 12-membered heteroaryl of R^{Za}, R^{Zb}, and R^{Zc} is optionally substituted with one or more halo, —OH, C₁₋₆ alkyl, —C(O)OR^{c}, or —C(O)NR^{c}R^{d};
each R^{c} and R^{d} is independently H or C₁₋₆ alkyl optionally substituted with one or more —OH, halo, —NH₂, —CF₃, or oxo;
Y is —NH— or —O—;
Ring A is selected from the group consisting of: 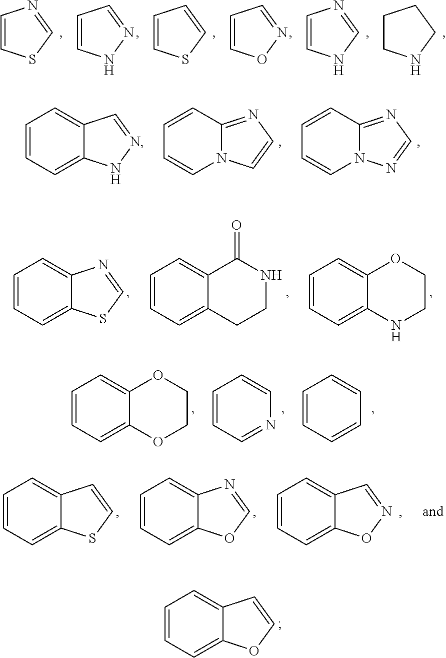
and
each T is independently selected from the group consisting of halo, —NH₂, C₁-C₆ alkyl, C₁₋₆alkoxy, —NHC(O)—C₃₋₆cycloalkyl, and methylpiperidyl,
provided that:
(1) n+w is an integer greater than 0, and
(2) when Ring A is phenyl, then either: (a) n is an integer from 1 to 5 and Ring B is substituted by at least one (p)ₘ-Z wherein Z is 3- to 12-membered heterocyclyl substituted with one or more R^{Za} or (b) Ring A is substituted by at least one T wherein T is methylpiperidyl.

3. The compound of claim 1, or a pharmaceutically acceptable salt, stereoisomer, or tautomer thereof, wherein Q is —NH—.

4. The compound of claim 1, or a pharmaceutically acceptable salt, stereoisomer, or tautomer thereof, wherein Qis a bond.

5. The compound of claim 1, or a pharmaceutically acceptable salt, stereoisomer, or tautomer thereof, wherein Ring B is C₆₋₁₄ aryl.

6. The compound of claim 5, wherein Ring B is phenyl.

7. The compound of claim 1, or a pharmaceutically acceptable salt, stereoisomer, or tautomer thereof, wherein Ring B is pyridyl.

8. The compound of claim 1, or a pharmaceutically acceptable salt, stereoisomer, or tautomer thereof, wherein q is an integer from 1-4 and at least one R¹ is halo.

9. The compound of claim 8, or a pharmaceutically acceptable salt, stereoisomer, or tautomer thereof, wherein R¹ is F.

10. The compound of claim 8, or a pharmaceutically acceptable salt, stereoisomer, or tautomer thereof, wherein R¹ is Cl.

11. The compound of claim 1, or a pharmaceutically acceptable salt, stereoisomer, or tautomer thereof, wherein q is an integer from 1-4 and at least one R¹ is —CN.

12. The compound of claim 1, or a pharmaceutically acceptable salt, stereoisomer, or tautomer thereof, wherein q is an integer from 1-4 and at least one R¹ is C₁₋₆ alkyl.

13. The compound of claim 1, or a pharmaceutically acceptable salt, stereoisomer, or tautomer thereof, wherein q is an integer from 1-4 and at least one R¹ is C₁₋₆ alkoxy.

14. The compound of claim 1, or a pharmaceutically acceptable salt, stereoisomer, or tautomer thereof, wherein d is an integer from 1-3.

15. The compound of claim 14, or a pharmaceutically acceptable salt, stereoisomer, or tautomer thereof, wherein at least one R² is F.

16. The compound of claim 14, or a pharmaceutically acceptable salt, stereoisomer, or tautomer thereof, wherein at least one R² is Cl.

17. The compound of claim 1, or a pharmaceutically acceptable salt, stereoisomer, or tautomer thereof, wherein Y is —NH—.

18. The compound of claim 1, or a pharmaceutically acceptable salt, stereoisomer, or tautomer thereof, wherein Y is —O—.

19. The compound of claim 1, or a pharmaceutically acceptable salt, stereoisomer, or tautomer thereof, wherein n is an integer from 1-5 and at least one Z is —NR^{c}R^{d}.

20. The compound of claim 1, or a pharmaceutically acceptable salt, stereoisomer, or tautomer thereof, wherein n is an integer from 1-5 and at least one Z is 3- to 12-membered heterocyclyl optionally substituted with one or more R^{Za}.

21. The compound of claim 20, or a pharmaceutically acceptable salt, stereoisomer, or tautomer thereof, wherein Z is substituted with one or more R^{Za} that is C₁₋₆alkyl.

22. The compound of claim 20, or a pharmaceutically acceptable salt, stereoisomer, or tautomer thereof, wherein Z is substituted with one or more R^{Za} that is oxo.

23. The compound of claim 1, or a pharmaceutically acceptable salt, stereoisomer, or tautomer thereof, wherein n is an integer from 1-5 and Z is 5- to 12-membered heteroaryl optionally substituted with one or more R^{Zb}.

24. The compound of claim 1, or a pharmaceutically acceptable salt, stereoisomer, or tautomer thereof, wherein n is an integer from 1-5 and Z is C₆₋₁₄ aryl, optionally substituted with one or more R^{Zc}.

25. The compound of claim 24, or a pharmaceutically acceptable salt, stereoisomer, or tautomer thereof, wherein Z is substituted with one or more R^{Zc} that is halo.

26. The compound of claim 1, wherein n is 1.

27. The compound of claim 1, or a pharmaceutically acceptable salt, stereoisomer, or tautomer thereof, wherein Ring A is

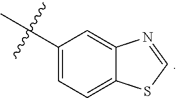 [SMILES: *c1ccc2scnc2c1]

28. The compound of claim 1, or a pharmaceutically acceptable salt, stereoisomer, or tautomer thereof, wherein Ring A is

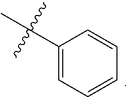 [SMILES: *c1ccccc1]

29. The compound of claim 1, or a pharmaceutically acceptable salt, stereoisomer, or tautomer thereof, wherein Ring A is

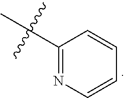 [SMILES: *c1ccccn1]

30. The compound of claim 1, or a pharmaceutically acceptable salt, stereoisomer, or tautomer thereof, wherein w is an integer from 1-5 and at least one T is halo.

31. The compound of claim 30, or a pharmaceutically acceptable salt, stereoisomer, or tautomer thereof, wherein at least one T is F.

32. The compound of claim 30, or a pharmaceutically acceptable salt, stereoisomer, or tautomer thereof, wherein at least one T is Cl.

33. The compound of claim 1, or a pharmaceutically acceptable salt, stereoisomer, or tautomer thereof, wherein w is an integer from 1-5 and at least one T is —NH₂.

34. The compound of claim 1, or a pharmaceutically acceptable salt, stereoisomer, or tautomer thereof, wherein w is an integer from 1-5 and at least one T is C₁-C₆ alkyl.

35. The compound of claim 1, or a pharmaceutically acceptable salt, stereoisomer, or tautomer thereof, wherein w is an integer from 1-5 and at least one T is NHC(O)—C₃₋₆cycloalkyl.

36. The compound of claim 1, or a pharmaceutically acceptable salt, stereoisomer, or tautomer thereof, wherein w is an integer from 1-5 and at least one T is methylpiperidyl.

37. The compound of claim 1, or a pharmaceutically acceptable salt, stereoisomer, or tautomer thereof, wherein w is an integer from 1-5 and at least one T is methoxy.

38. The compound of claim 1, or a pharmaceutically acceptable salt, stereoisomer, or tautomer thereof, wherein w is an integer from 1-3.

39. The compound of claim 1, or a pharmaceutically acceptable salt, stereoisomer, or tautomer thereof, wherein w is 0.

40. The compound of claim 1, or a pharmaceutically acceptable salt, stereoisomer, or tautomer thereof, wherein T is selected from the group consisting of —Cl, —F, —NH₂, —CH₃, 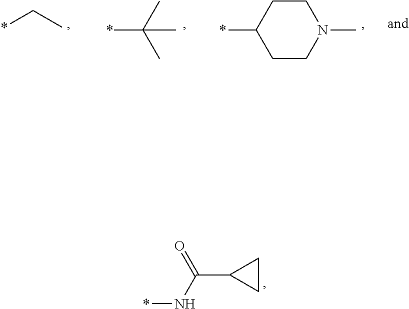
wherein, for each T, * denotes the point of attachment to Ring A.

41. The compound of claim 1, or a pharmaceutically acceptable salt, stereoisomer, or tautomer thereof, wherein Z is selected from the group consisting of 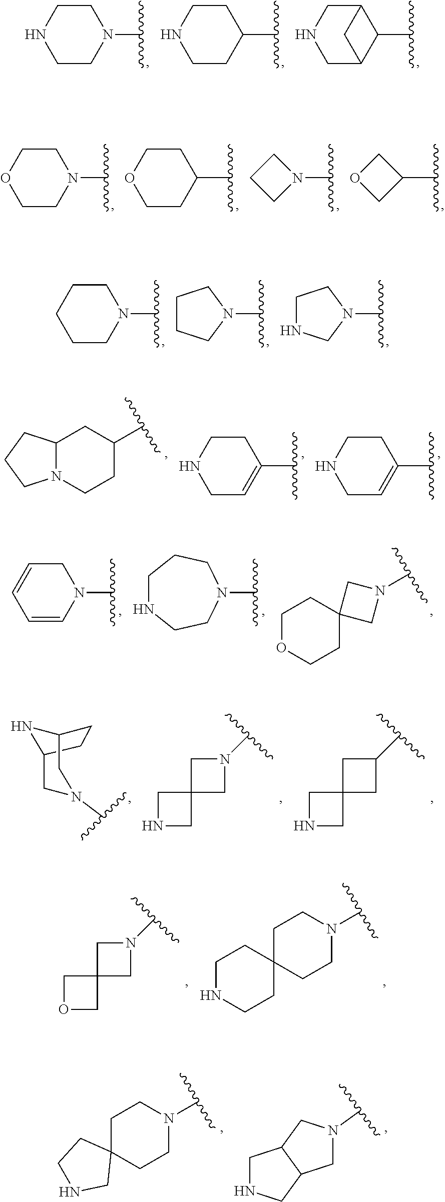 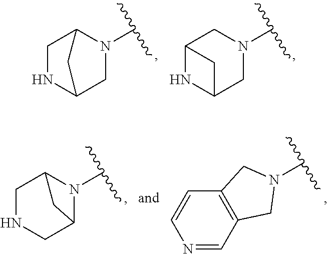 [SMILES: *N1CC2CC1CN2 ; *N1CC2CC(C1)N2 ; *N1C2CNCC1C2 ; *N1Cc2ccncc2C1]
each of which is optionally substituted with one or more R^{Za}; 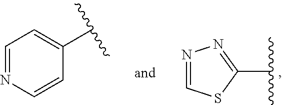 [SMILES: *c1ccncc1 ; *c1nncs1]
each of which is optionally substituted with one or more R^{Zb}; 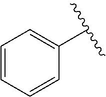 [SMILES: *c1ccccc1]
optionally substituted with one or more R^{Zc}; and 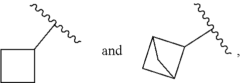 [SMILES: *C1CCC1 ; *C1C2CC1C2]
each of which is optionally substituted with one or more R^{Zd}.

42. The compound of claim 1, or a pharmaceutically acceptable salt, stereoisomer, or tautomer thereof, wherein R^{Za}, R^{Zb}, R^{Zc}, or R^{Zd} is selected from the group consisting of ═O, —O, —Cl, —F, —OH, —CH₃, —CF₃, NH₂,

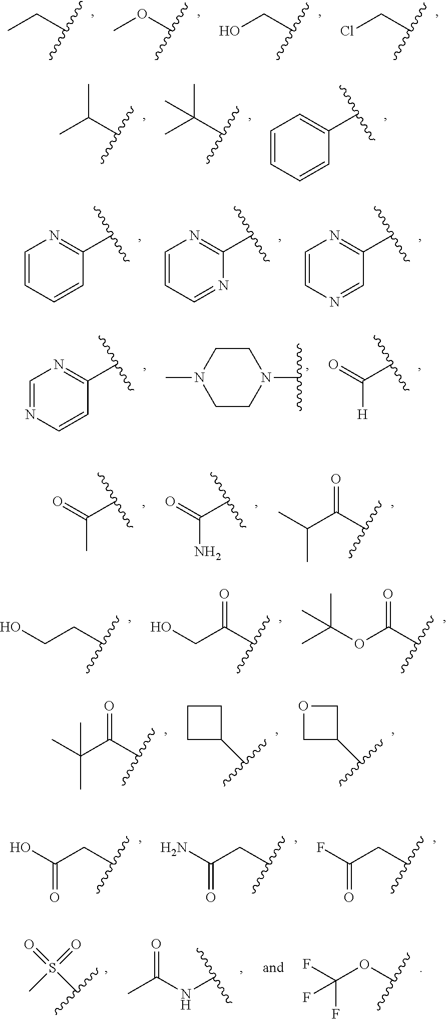

43. The compound of claim 1, or a pharmaceutically acceptable salt, stereoisomer, or tautomer thereof, wherein [Z-(p)ₘ] is selected from the group consisting of 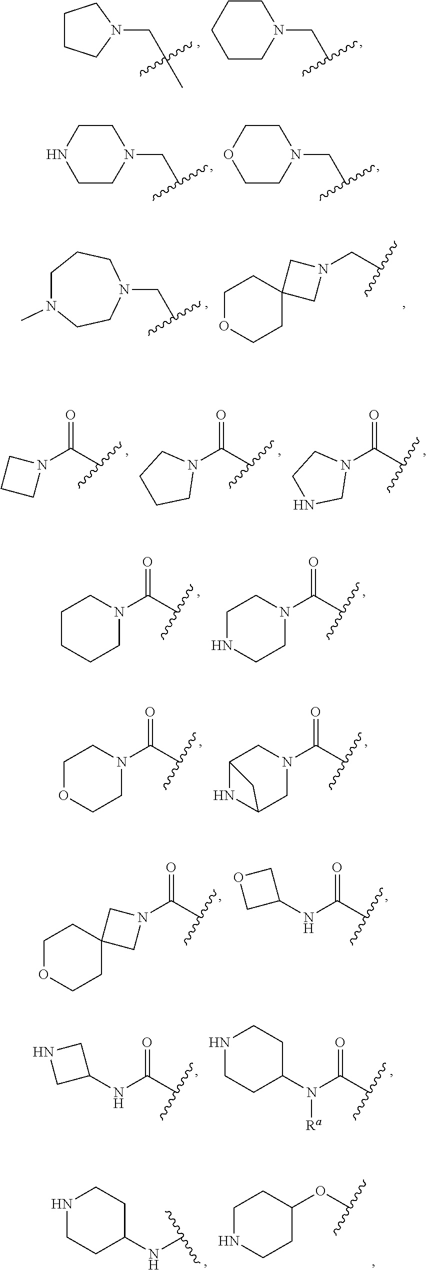 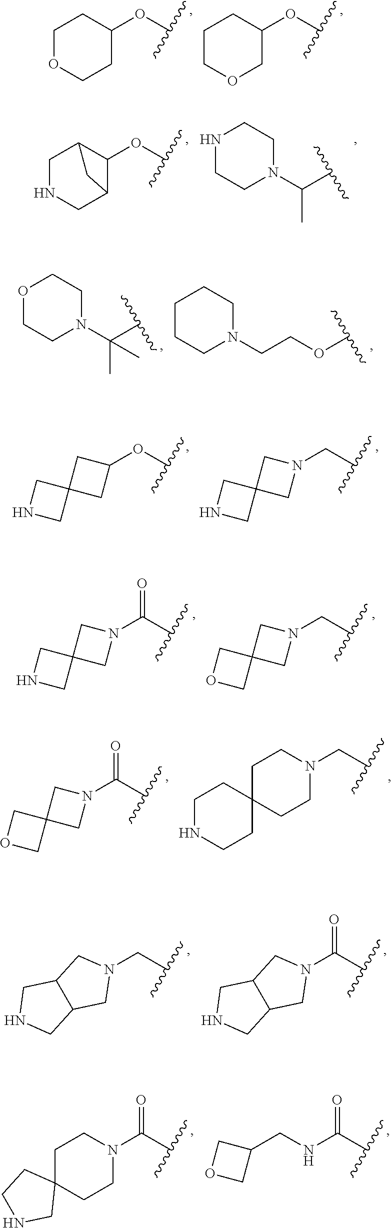 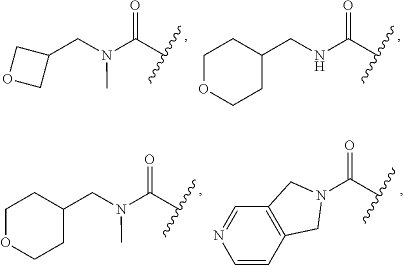
each of which is optionally substituted with one or more R^{Za}; 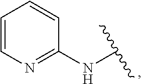 [SMILES: *Nc1ccccn1]
each of which is optionally substituted with one or more R^{Zb}; 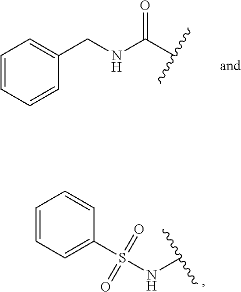 [SMILES: *C(=O)NCc1ccccc1 ; *NS(=O)(=O)c1ccccc1]
each of which is optionally substituted with one or more R^{Zc}; 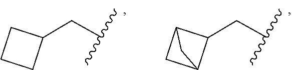 [SMILES: *CC1CCC1 ; *CC1C2CC1C2]
each of which is optionally substituted with one or more R^{Zd};

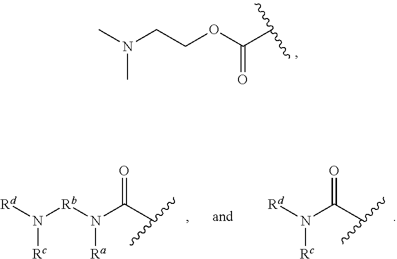

44. The compound of claim 1, or a pharmaceutically acceptable salt, stereoisomer, or tautomer thereof, wherein the compound is of the formula (II-1):

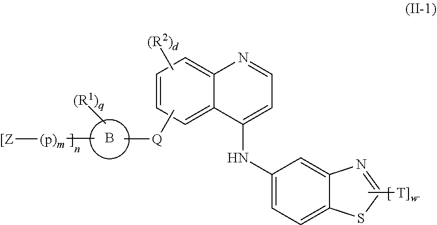

45. The compound of claim 1, or a pharmaceutically acceptable salt, stereoisomer, or tautomer thereof, wherein the compound is of the formula (II-2):

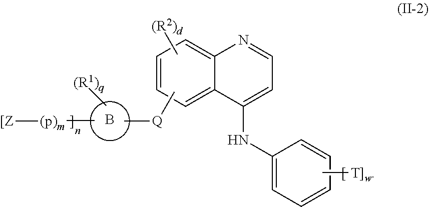

46. The compound of claim 1, or a pharmaceutically acceptable salt, stereoisomer, or tautomer thereof, wherein the compound is of the formula (III-1):

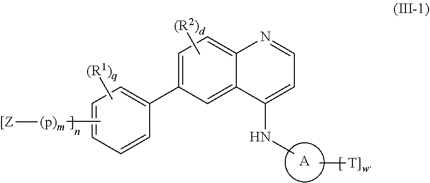

47. The compound of claim 1, or a pharmaceutically acceptable salt, stereoisomer, or tautomer thereof, wherein the compound is of the formula (III-2):

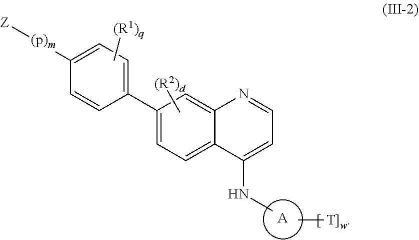

48. The compound of claim 1, or a pharmaceutically acceptable salt, stereoisomer, or tautomer thereof, wherein the compound is of the formula (IV-1):

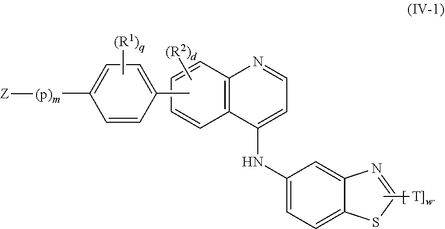

49. The compound of claim 1, or a pharmaceutically acceptable salt, stereoisomer, or tautomer thereof, wherein the compound is of the formula (IV-2):

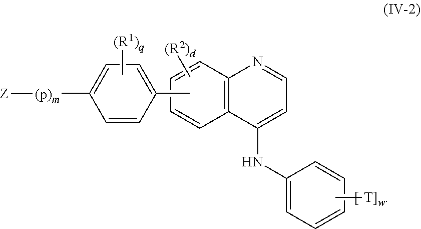

50. The compound of claim 1, or a pharmaceutically acceptable salt, stereoisomer, or tautomer thereof, wherein the compound is a compound of Table 1.

51. A pharmaceutical composition comprising the compound of claim 1, or a pharmaceutically acceptable salt, stereoisomer, or tautomer thereof, and a pharmaceutically acceptable carrier.

52. A method of inhibiting RIPK3 comprising contacting the kinase with a compound of claim 1 or a pharmaceutically acceptable salt, stereoisomer, or tautomer thereof.

53. A method of treating a disease or condition mediated by inhibition of RIPK3 in an individual in need thereof comprising administering to the individual a compound of claim 1, or a pharmaceutically acceptable salt, stereoisomer, or tautomer thereof.

54. The method of claim 53, wherein the disease or condition mediated by the inhibition of RIPK3 is a disease or condition is a systematic inflammatory response, an autoimmune disease, a proliferative disorder, a metabolic disease, a neurodegenerative disease or pain.

55. The method of claim 54, wherein the disease or condition is an age-related neurodegenerative disease or condition.
